SEQUENCE LISTING (832 sequences)

SEQ ID NO: 1 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - protein, 987 aa, synthetic construct
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNAYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDGSLQ

SEQ ID NO: 4 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 5 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 6 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 7 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVAVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 8 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 9 - protein, 492 aa, synthetic construct
MAGKKKDKDVINKTLSVRIIRPRYSDDIEKEISDEKAKRKQDGKTGELDRAFFSELKSRNPDIITNDELFPLFTEIQKNLTEIYNKSISLLYMKLIVEEEGGSTASALSAGPYKECKARFNSYISLGLRQKIQSNFRRKELKGFQVSLPTAKSDRFPIPFCHQVENGKGGFKVYETGDDFIFEVPLIKYTATNKKSTSGKNYTKVQLNNPPVPMNVPLLLSTMRRRQTKKGMQWNKDEGTNAELRRVMSGEYKVSYAEIIRRTRFGKHDDWFVNFSIKFKNKTDELNQNVRGGIDIGVSNPLVCAVTNGLDRYIVANNDIMAFNERAMARRRTLLRKNRFKRSGHGAKNKLEPITVLTEKNERFRKSILQRWAREVAEFFKRTSASVVNMEDLSGITEREDFFSTKLRTTWNYRLMQTTIENKLKEYGIAVNYISPKYTSQTCHSCGKRNDYFTFSYRSENNYPPFECKECNKVKCNADFNAAKNIALKVVL

SEQ ID NO: 10 - protein, 1367 aa, synthetic construct; identical to SEQ ID NO: 5

SEQ ID NO: 11 - protein, 840 aa, synthetic construct
MLKHKRKNKNSLARVVLSNYDSNNIYEIKIKYEKLAKLDKINIIEMDYDADNNVMFKKVLFNNKEIDLSHKDKTKINIELDNKKYNISAKKQIGKTHLVVRDKQTSKISRIKKIQDTYYRGKDVFILDNNIEILDKKQTKDKFIVTLNDITNDKTTSTEAELIDDTKDIFKKISAKKDLKSSDIYKIKRFISIRSNFSFYYTFVDNYFKIFHAKKDKNKEELYKIKFKDEINIKPYLENILDNMKNKNGILYDYADDREKVLNDLKNIQYVFTEFRHKLAHFDYNFLDNFFSNSVTDQYKQKVNEIKLLDILLDNIDSLNVVPKQNYIEDETISVFDAKDIKLKRLYTYYIKLTINYPGFKKLINSFFIQDGIENQELKEYINNKEKDTQVLKELDNKAYYMDISQYRKYKNIYNKHKELVSEKELSSDGQKINSLNQKINKLKIEMKNITKPNALNRLIYRLRVAFGFIYKEYATINNFNKSFLQDTKIKRFENISQQDIKNYLDISYQDKGKFFVKSKKTFKNKTTIKYTFEDLDLTLNEIITQDDIFVKVIFLFSIFMPKELNGDFFGFINMYYHKMKNISYDTKDIDMLDTISQNMKLKILEQNIKKTYVFKYYLDLDSSIYSKLVQNIKITEDIDSKKYLYAKIFKYYQHLYKLISDVEIYLLYKYNSKENLSITIDKDELKHRGYYNFQSLLIKNNINKDDAYWSIVNMRNNLSHQNIDELVGHFCKGCLRKSTTDIAELWLRKDILTITNEIINKIESFKDIKITLGYDCVNDFTQKVKQYKQKLKASNERLAKKIEEKQNQVVDEKNKEELEKKILNMKNIQKINRYILDIL

SEQ ID NO: 12 - protein, 707 aa, synthetic construct
MADTPTLFTQFLRHHLPGQRFRKDILKQAGRILANKGEDATIAFLRGKSEESPPDFQPPVKCPIIACSRPLTEWPIYQASVAIQGYVYGQSLAEFEASDPGCSKDGLLGWFDKTGVCTDYFSVQGLNLIFQNARKRYIGVQTKVTNRNEKRHKKLKRINAKRIAEGLPELTSDEPESALDETGHLIDPPGLNTNIYCYQQVSPKPLALSEVNQLPTAYAGYSTSGDDPIQPMVTKDRLSISKGQPGYIPEHQRALLSQKKHRRMRGYGLKARALLVIVRIQDDWAVIDLRSLLRNAYWRRIVQTKEPSTITKLLKLVTGDPVLDATRMVATFTYKPGIVQVRSAKCLKNKQGSKLFSERYLNETVSVTSIDLGSNNLVAVATYRLVNGNTPELLQRFTLPSHLVKDFERYKQAHDTLEDSIQKTAVASLPQGQQTEIRMWSMYGFREAQERVCQELGLADGSIPWNVMTATSTILTDLFLARGGDPKKCMFTSEPKKKKNSKQVLYKIRDRAWAKMYRTLLSKETREAWNKALWGLKRGSPDYARLSKRKEELARRCVNYTISTAEKRAQCGRTIVALEDLNIGFFHGRGKQEPGWVGLFTRKKENRWLMQALHKAFLELAHHRGYHVIEVNPAYTSQTCPVCRHCDPDNRDQHNREAFHCIGCGFRGNADLDVATHNIAMVAITGESLKRARGSVASKTPQPLAAE

SEQ ID NO: 13 - protein, 757 aa, synthetic construct
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 14 - protein, 765 aa, synthetic construct
MYILEMADLKSEPSLLAKLLRDRFPGKYWLPKYWKLAEKKRLTGGEEAACEYMADKQLDSPPPNFRPPARCVILAKSRPFEDWPVHRVASKAQSFVIGLSEQGFAALRAAPPSTADARRDWLRSHGASEDDLMALEAQLLETIMGNAISLHGGVLKKIDNANVKAAKRLSGRNEARLNKGLQELPPEQEGSAYGADGLLVNPPGLNLNIYCRKSCCPKPVKNTARFVGHYPGYLRDSDSILISGTMDRLTIIEGMPGHIPAWQREQGLVKPGGRRRRLSGSESNMRQKVDPSTGPRRSTRSGTVNRSNQRTGRNGDPLLVEIRMKEDWVLLDARGLLRNLRWRESKRGLSCDHEDLSLSGLLALFSGDPVIDPVRNEVVFLYGEGIIPVRSTKPVGTRQSKKLLERQASMGPLTLISCDLGQTNLIAGRASAISLTHGSLGVRSSVRIELDPEIIKSFERLRKDADRLETEILTAAKETLSDEQRGEVNSHEKDSPQTAKASLCRELGLHPPSLPWGQMGPSTTFIADMLISHGRDDDAFLSHGEFPTLEKRKKFDKRFCLESRPLLSSETRKALNESLWEVKRTSSEYARLSQRKKEMARRAVNFVVEISRRKTGLSNVIVNIEDLNVRIFHGGGKQAPGWDGFFRPKSENRWFIQAIHKAFSDLAAHHGIPVIESDPQRTSMTCPECGHCDSKNRNGVRFLCKGCGASMDADFDAACRNLERVALTGKPMPKPSTSCERLLSATTGKVCSDHSLSHDAIEKAS

SEQ ID NO: 15 - protein, 766 aa, synthetic construct
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKAFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 16 - protein, 812 aa, synthetic construct
MDMLDTETNYATETPAQQQDYSPKPPKKAQRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVNIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 17 - protein, 812 aa, synthetic construct
MDMLDTETNYATETPAQQQDYSPKPPKKAQRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVDIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHKGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 18 - protein, 793 aa, synthetic construct
MSSLPTPLELLKQKHADLFKGLQFSSKDNKMAGKVLKKDGEEAALAFLSERGVSRGELPNFRPPAKTLVVAQSRPFEEFPIYRVSEAIQLYVYSLSVKELETVPSGSSTKKEHQRFFQDSSVPDFGYTSVQGLNKIFGLARGIYLGVITRGENQLQKAKSKHEALNKKRRASGEAETEFDPTPYEYMTPERKLAKPPGVNHSIMCYVDISVDEFDFRNPDGIVLPSEYAGYCREINTAIEKGTVDRLGHLKGGPGYIPGHQRKESTTEGPKINFRKGRIRRSYTALYAKRDSRRVRQGKLALPSYRHHMMRLNSNAESAILAVIFFGKDWVVFDLRGLLRNVRWRNLFVDGSTPSTLLGMFGDPVIDPKRGVVAFCYKEQIVPVVSKSITKMVKAPELLNKLYLKSEDPLVLVAIDLGQTNPVGVGVYRVMNASLDYEVVTRFALESELLREIESYRQRTNAFEAQIRAETFDAMTSEEQEEITRVRAFSASKAKENVCHRFGMPVDAVDWATMGSNTIHIAKWVMRHGDPSLVEVLEYRKDNEIKLDKNGVPKKVKLTDKRIANLTSIRLRFSQETSKHYNDTMWELRRKHPVYQKLSKSKADFSRRVVNSIIRRVNHLVPRARIVFIIEDLKNLGKVFHGSGKRELGWDSYFEPKSENRWFIQVLHKAFSETGKHKGYYIIECWPNWTSCTCPKCSCCDSENRHGEVFRCLACGYTCNTDFGTAPDNLVKIATTGKGLPGPKKRCKGSSKGKNPKIARSSETGVSVTESGAPKVKKSSPTQTSQSSSQSAP

SEQ ID NO: 19 - protein, 441 aa, synthetic construct
MNKIEKEKTPLAKLMNENFAGLRFPFAIIKQAGKKLLKEGELKTIEYMTGKGSIEPLPNFKPPVKCLIVAKRRDLKYFPICKASCEIQSYVYSLNYKDFMDYFSTPMTSQKQHEEFFKKSGLNIEYQNVAGLNLIFNNVKNTYNGVILKVKNRNEKLKKKAIKNNYEFEEIKTFNDDGCLINKPGINNVIYCFQSISPKILKNITHLPKEYNDYDCSVDRNIIQKYVSRLDIPESQPGHVPEWQRKLPEFNNTNNPRRRRKWYSNGRNISKGYSVDQVNQAKIEDSLLAQIKIGEDWIILDIRGLLRDLNRRELISYKNKLTIKDVLGFFSDYPIIDIKKNLVTFCYKEGVIQVVSQKSIGNKKSKQLLEKLIENKPIALVSIDLGQTNPVSVKISKLNKINNKISIESFTYRFLNEEILKEIEKYRKDYDKLELKLINEA

SEQ ID NO: 20 - protein, 812 aa, synthetic construct
MDMLDTETNYATETPSQQQDYSPKPPKKDRRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVDIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 21 - protein, 812 aa, synthetic construct
MDMLDTETNYATETPSQQQDYSPKPPKKDRRAPKGFSKKARPEKKPPKPITLFTQKHFSGVRFLKRVIRDASKILKLSESRTITFLEQAIERDGSAPPDVTPPVHNTIMAVTRPFEEWPEVILSKALQKHCYALTKKIKIKTWPKKGPGKKCLAAWSARTKIPLIPGQVQATNGLFDRIGSIYDGVEKKVTNRNANKKLEYDEAIKEGRNPAVPEYETAYNIDGTLINKPGYNPNLYITQSRTPRLITEADRPLVEKILWQMVEKKTQSRNQARRARLEKAAHLQGLPVPKFVPEKVDRSQKIEIRIIDPLDKIEPYMPQDRMAIKASQDGHVPYWQRPFLSKRRNRRVRAGWGKQVSSIQAWLTGALLVIVRLGNEAFLADIRGALRNAQWRKLLKPDATYQSLFNLFTGDPVVNTRTNHLTMAYREGVVNIVKSRSFKGRQTREHLLTLLGQGKTVAGVSFDLGQKHAAGLLAAHFGLGEDGNPVFTPIQACFLPQRYLDSLTNYRNRYDALTLDMRRQSLLALTPAQQQEFADAQRDPGGQAKRACCLKLNLNPDEIRWDLVSGISTMISDLYIERGGDPRDVHQQVETKPKGKRKSEIRILKIRDGKWAYDFRPKIADETRKAQREQLWKLQKASSEFERLSRYKINIARAIANWALQWGRELSGCDIVIPVLEDLNVGSKFFDGKGKWLLGWDNRFTPKKENRWFIKVLHKAVAELAPHRGVPVYEVMPHRTSMTCPACHYCHPTNREGDRFECQSCHVVKNTDRDVAPYNILRVAVEGKTLDRWQAEKKPQAEPDRPMILIDNQES

SEQ ID NO: 22 - protein, 761 aa, synthetic construct
MSNKTTPPSPLSLLLRAHFPGLKFESQDYKIAGKKLRDGGPEAVISYLTGKGQAKLKDVKPPAKAFVIAQSRPFIEWDLVRVSRQIQEKIFGIPATKGRPKQDGLSETAFNEAVASLEVDGKSKLNEETRAAFYEVLGLDAPSLHAQAQNALIKSAISIREGVLKKVENRNEKNLSKTKRRKEAGEEATFVEEKAHDERGYLIHPPGVNQTIPGYQAVVIKSCPSDFIGLPSGCLAKESAEALTDYLPHDRMTIPKGQPGYVPEWQHPLLNRRKNRRRRDWYSASLNKPKATCSKRSGTPNRKNSRTDQIQSGRFKGAIPVLMRFQDEWVIIDIRGLLRNARYRKLLKEKSTIPDLLSLFTGDPSIDMRQGVCTFIYKAGQACSAKMVKTKNAPEILSELTKSGPVVLVSIDLGQTNPIAAKVSRVTQLSDGQLSHETLLRELLSNDSSDGKEIARYRVASDRLRDKLANLAVERLSPEHKSEILRAKNDTPALCKARVCAALGLNPEMIAWDKMTPYTEFLATAYLEKGGDRKVATLKPKNRPEMLRRDIKFKGTEGVRIEVSPEAAEAYREAQWDLQRTSPEYLRLSTWKQELTKRILNQLRHKAAKSSQCEVVVMAFEDLNIKMMHGNGKWADGGWDAFFIKKRENRWFMQAFHKSLTELGAHKGVPTIEVTPHRTSITCTKCGHCDKANRDGERFACQKCGFVAHADLEIATDNIERVALTGKPMPKPESERSGDAKKSVGARKAAFKPEEDAEAAE

SEQ ID NO: 23 - protein, 781 aa, synthetic construct
MRQPAEKTAFQVFRQEVIGTQKLSGGDAKTAGRLYKQGKMEAAREWLLKGARDDVPPNFQPPAKCLVVAVSHPFEEWDISKTNHDVQAYIYAQPLQAEGHLNGLSEKWEDTSADQHKLWFEKTGVPDRGLPVQAINKIAKAAVNRAFGVVRKVENRNEKRRSRDNRIAEHNRENGLTEVVREAPEVATNADGFLLHPPGIDPSILSYASVSPVPYNSSKHSFVRLPEEYQAYNVEPDAPIPQFVVEDRFAIPPGQPGYVPEWQRLKCSTNKHRRMRQWSNQDYKPKAGRRAKPLEFQAHLTRERAKGALLVVMRIKEDWVVFDVRGLLRNVEWRKVLSEEAREKLTLKGLLDLFTGDPVIDTKRGIVTFLYKAEITKILSKRTVKTKNARDLLLRLTEPGEDGLRREVGLVAVDLGQTHPIAAAIYRIGRTSAGALESTVLHRQGLREDQKEKLKEYRKRHTALDSRLRKEAFETLSVEQQKEIVTVSGSGAQITKDKVCNYLGVDPSTLPWEKMGSYTHFISDDFLRRGGDPNIVHFDRQPKKGKVSKKSQRIKRSDSQWVGRMRPRLSQETAKARMEADWAAQNENEEYKRLARSKQELARWCVNTLLQNTRCITQCDEIVVVIEDLNVKSLHGKGAREPGWDNFFTPKTENRWFIQILHKTFSELPKHRGEHVIEGCPLRTSITCPACSYCDKNSRNGEKFVCVACGATFHADFEVATYNLVRLATTGMPMPKSLERQGGGEKAGGARKARKKAKQVEKIVVQANANVTMNGASLHSP

SEQ ID NO: 24 - protein, 793 aa, synthetic construct; identical to SEQ ID NO: 18

SEQ ID NO: 25 - protein, 717 aa, synthetic construct; identical to SEQ ID NO: 6

SEQ ID NO: 26 - protein, 761 aa, synthetic construct; identical to SEQ ID NO: 22

SEQ ID NO: 27 - protein, 765 aa, synthetic construct
MYSLEMADLKSEPSLLAKLLRDRFPGKYWLPKYWKLAEKKRLTGGEEAACEYMADKQLDSPPPNFRPPARCVILAKSRPFEDWPVHRVASKAQSFVIGLSEQGFAALRAAPPSTADARRDWLRSHGASEDDLMALEAQLLETIMGNAISLHGGVLKKIDNANVKAAKRLSGRNEARLNKGLQELPPEQEGSAYGADGLLVNPPGLNLNIYCRKSCCPKPVKNTARFVGHYPGYLRDSDSILISGTMDRLTIIEGMPGHIPAWQREQGLVKPGGRRRRLSGSESNMRQKVDPSTGPRRSTRSGTVNRSNQRTGRNGDPLLVEIRMKEDWVLLDARGLLRNLRWRESKRGLSCDHEDLSLSGLLALFSGDPVIDPVRNEVVFLYGEGIIPVRSTKPVGTRQSKKLLERQASMGPLTLISCDLGQTNLIAGRASAISLTHGSLGVRSSVRIELDPEIIKSFERLRKDADRLETEILTAAKETLSDEQRGEVNSHEKDSPQTAKASLCRELGLHPPSLPWGQMGPSTTFIADMLISHGRDDDAFLSHGEFPTLEKRKKFDKRFCLESRPLLSSETRKALNESLWEVKRTSSEYARLSQRKKEMARRAVNFVVEISRRKTGLSNVIVNIEDLNVRIFHGGGKQAPGWDGFFRPKSENRWFIQAIHKAFSDLAAHHGIPVIESDPQRTSMTCPECGHCDSKNRNGVRFLCKGCGASMDADFDAACRNLERVALTGKPMPKPSTSCERLLSATTGKVCSDHSLSHDAIEKAS

SEQ ID NO: 28 - protein, 766 aa, synthetic construct
MEKEITELTKIRREFPNKKFSSTDMKKAGKLLKAEGPDAVRDFLNSCQEIIGDFKPPVKTNIVSISRPFEEWPVSMVGRAIQEYYFSLTKEELESVHPGTSSEDHKSFFNITGLSNYNYTSVQGLNLIFKNAKAIYDGTLVKANNKNKKLEKKFNEINHKRSLEGLPIITPDFEEPFDENGHLNNPPGINRNIYGYQGCAAKVFVPSKHKMVSLPKEYEGYNRDPNLSLAGFRNRLEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGVVTFSYKEGVVPVFSQKIVPRFKSRDTLEKLTSQGPVALLSVDLGQNEPVAARVCSLKNINDKITLDNSCRISFLDDYKKQIKDYRDSLDELEIKIRLEAINSLETNQQVEIRDLDVFSADRAKANTVDMFDIDPNLISWDSMSDARVSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKTFSELSSNRGLCVIEVNPAWTSATCPDCGFCSKENRDGINFTCRKCGVSYHADIDVATLNIARVAVLGKPMSGPADRERLGDTKKPRVARSRKTMKRKDISNSTVEAMVTA

SEQ ID NO: 29 - protein, 870 aa, synthetic construct
MLVRTSTLVQDNKNSRSASRAFLKKPKMPKNKHIKEPTELAKLIRELFPGQRFTRAINTQAGKILKHKGRDEVVEFLKNKGIDKEQFMDFRPPTKARIVATSGAIEEFSYLRVSMAIQECCFGKYKFPKEKVNGKLVLETVGLTKEELDDFLPKKYYENKKSRDRFFLKTGICDYGYTYAQGLNEIFRNTRAIYEGVFTKVNNRNEKRREKKDKYNEERRSKGLSEEPYDEDESATDESGHLINPPGVNLNIWTCEGFCKGPYVTKLSGTPGYEVILPKVFDGYNRDPNEIISCGITDRFAIPEGEPGHIPWHQRLEIPEGQPGYVPGHQRFADTGQNNSGKANPNKKGRMRKYYGHGTKYTQPGEYQEVFRKGHREGNKRRYWEEDFRSEAHDCILYVIHIGDDWVVCDLRGPLRDAYRRGLVPKEGITTQELCNLFSGDPVIDPKHGVVTFCYKNGLVRAQKTISAGKKSRELLGALTSQGPIALIGVDLGQTEPVGARAFIVNQARGSLSLPTLKGSFLLTAENSSSWNVFKGEIKAYREAIDDLAIRLKKEAVATLSVEQQTEIESYEAFSAEDAKQLACEKFGVDSSFILWEDMTPYHTGPATYYFAKQFLKKNGGNKSLIEYIPYQKKKSKKTPKAVLRSDYNIACCVRPKLLPETRKALNEAIRIVQKNSDEYQRLSKRKLEFCRRVVNYLVRKAKKLTGLERVIIAIEDLKSLEKFFTGSGKRDNGWSNFFRPKKENRWFIPAFHKAFSELAPNRGFYVIECNPARTSITDPDCGYCDGDNRDGIKFECKKCGAKHHTDLDVAPLNIAIVAVTGRPMPKTVSNKSKRERSGGEKSVGASRKRNHRKSKANQEMLDATSSAAE

SEQ ID NO: 30 - protein, 830 aa, synthetic construct
MPKIKKPTEISLLRKEVFPDLHFAKDRMRAASLVLKNEGREAAIEYLRVNHEDKPPNFMPPAKTPYVALSRPLEQWPIAQASIAIQKYIFGLTKDEFSATKKLLYGDKSTPNTESRKRWFEVTGVPNFGYMSAQGLNAIFSGALARYEGVVQKVENRNKKRFEKLSEKNQLLIEEGQPVKDYVPDTAYHTPETLQKLAENNHVRVEDLGDMIDRLVHPPGIHRSIYGYQQVPPFAYDPDNPKGIILPKAYAGYTRKPHDIIEAMPNRLNIPEGQAGYIPEHQRDKLKKGGRVKRLRTTRVRVDATETVRAKAEALNAEKARLRGKEAILAVFQIEEDWALIDMRGLLRNVYMRKLIAAGELTPTTLLGYFTETLTLDPRRTEATFCYHLRSEGALHAEYVRHGKNTRELLLDLTKDNEKIALVTIDLGQRNPLAAAIFRVGRDASGDLTENSLEPVSRMLLPQAYLDQIKAYRDAYDSFRQNIWDTALASLTPEQQRQILAYEAYTPDDSKENVLRLLLGGNVMPDDLPWEDMTKNTHYISDRYLADGGDPSKVWFVPGPRKRKKNAPPLKKPPKPRELVKRSDHNISHLSEFRPQLLKETRDAFEKAKIDTERGHVGYQKLSTRKDQLCKEILNWLEAEAVRLTRCKTMVLGLEDLNGPFFNQGKGKVRGWVSFFRQKQENRWIVNGFRKNALARAHDKGKYILELWPSWTSQTCPKCKHVHADNRHGDDFVCLQCGARLHADAEVATWNLAVVAIQGHSLPGPVREKSNDRKKSGSARKSKKANESGKVVGAWAAQATPKRATSKKETGTARNPVYNPLETQASCPAP

SEQ ID NO: 31 - protein, 790 aa, synthetic construct
MTPSPQIARLVETPLAAALKAHHPGKKFRSDYLKKAGKILKDQGVEAAMAHLDGKDQAEPPNFKPPAKCRIVARSREFSEWPIVKASVEIQKYIYGLTLEERKACDPGKSSASHKAWFAKTGVNTFGYSSVQGFNLIFGHTLGRYDGVLVKTENLNKKRAEKNERFRAKALAEGRAEPVCPPLVTATNDTGQDVTLEDGRVVRPGQLLQPPGINPNIYAYQQVSPKAYVPGIIELPEEFQGYSRDPNAVILPLVPRDRLSIPKGQPGYVPEPHREGLTGRKDRRMRRYYETERGTKLKRPPLTAKGRADKANEALLVVVRIDSDWVVMDVRGLLRNARWRRLVSKEGITLNGLLDLFTGDPVLNPKDCSVSRDTGDPVNDPRHGVVTFCYKLGVVDVCSKDRPIKGFRTKEVLERLTSSGTVGMVSIDLGQTNPVAAAVSRVTKGLQAETLETFTLPDDLLGKVRAYRAKTDRMEEGFRRNALRKLTAEQQAEITRYNDATEQQAKALVCSTYGIGPEEVPWERMTSNTTYISDHILDHGGDPDTVFFMATKRGQNKPTLHKRKDKAWGQKFRPAISVETRLARQAAEWELRRASLEFQKLSVWKTELCRQAVNYVMERTKKRTQCDVIIPVIEDLPVPLFHGSGKRDPGWANFFVHKRENRWFIDGLHKAFSELGKHRGIYVFEVCPQRTSITCPKCGHCDPDNRDGEKFVCLSCQATLNADLDVATTNLVRVALTGKVMPRSERSGDAQTPGPARKARTGKIKGSKPTSAPQGATQTDAKAHLSQTGV

SEQ ID NO: 32 - protein, 790 aa, synthetic construct
MTPSPQIARLVETPLAAALKAHHPGKKFRSDYLKKAGKILKDQGVEAAMAHLDGKDQAEPPNFKPPAKCRIVARSREFSEWPIVKASVEIQKYIYGLTLEERKACDPGKSSASHKAWFAKTGVNTFGYSSVQGFNLIFGHTLGRYDGVLVKTENLNKKRAEKNERFRAKALAEGRAEPVCPPLVTATNDTGQDVTLEDGRVVRPGQLLQPPGINPNIYAYQQVSPKAYVPGIIELPEEFQGYSRDPNAVILPLVPRDRLSIPKGQPGYVPEPHREGLTGRKDRRMRRYYETERGTKLKRPPLTAKGRADKANEALLVVVRIDSDWVVMDVRGLLRNARWRRLVSKEGITLNGLLDLFTGDPVLNPKDCSVSRDTGDPVNDPRHGVVTFCYKLGVVDVCSKDRPIKGFRTKEVLERLTSSGTVGMVSIDLGQTNPVAAAVSRVTKGLQAETLETFTLPDDLLGKVRAYRAKTDRMEEGFRRNALRKLTAEQQAEITRYNDATEQQAKALVCSTYGIGPEEVPWERMTSNTTYISDHILDHGGDPDTVFFMATKRGQNKPTLHKRKDKAWGQKFRPAISVETRLARQAAEWELRRASLEFQKLSVWKTELCRQAVNYVMERTKKRTQCDVIIPVIEDLPVPLFHGSGKRDPGWANFFVHKRENRWFIDGLHKAFSELGKHRGIYVFEVCPQRTSITCPKCGHCDPDNRDGEKFVCLSCQATLHADLDVATTNLVRVALTGKVMPRSERSGDAQTPGPARKARTGKIKGSKPTSAPQGATQTDAKAHLSQTGV

SEQ ID NO: 33 - protein, 782 aa, synthetic construct
MKTEKPKTALTLLREEVFPGKKYRLDVLKEAGKKLSTKGREATIEFLTGKDEERPQNFQPPAKTSIVAQSRPFDQWPIVQVSLAVQKYIYGLTQSEFEANKKALYGETGKAISTESRRAWFEATGVDNFGFTAAQGINPIFSQAVARYEGVIKKVENRNEKKLKKLTKKNLLRLESGEEIEDFEPEATFNEEGRLLQPPGANPNIYCYQQISPRIYDPSDPKGVILPQIYAGYDRKPEDIISAGVPNRLAIPEGQPGYIPEHQRAGLKTQGRIRCRASVEAKARAAILAVVHLGEDWVVLDLRGLLRNVYWRKLASPGTLTLKGLLDFFTGGPVLDARRGIATFSYTLKSAAAVHAENTYKGKGTREVLLKLTENNSVALVTVDLGQRNPLAAMIARVSRTSQGDLTYPESVEPLTRLFLPDPFLEEVRKYRSSYDALRLSIREAAIASLTPEQQAEIRYIEKFSAGDAKKNVAEVFGIDPTQLPWDAMTPRTTYISDLFLRMGGDRSRVFFEVPPKKAKKAPKKPPKKPAGPRIVKRTDGMIARLREIRPRLSAETNKAFQEARWEGERSNVAFQKLSVRRKQFARTVVNHLVQTAQKMSRCDTVVLGIEDLNVPFFHGRGKYQPGWEGFFRQKKENRWLINDMHKALSERGPHRGGYVLELTPFWTSLRCPKCGHTDSANRDGDDFVCVKCGAKLHSDLEVATANLALVAITGQSIPRPPREQSSGKKSTGTARMKKTSGETQGKGSKACVSEALNKIEQGTARDPVYNPLNSQVSCPAP

SEQ ID NO: 34 - protein, 774 aa, synthetic construct
VYNPDMKKPNNIRRIREEHFEGLCFGKDVLTKAGKIYEKDGEEAAIDFLMGKDEEDPPNFKPPAKTTIVAQSRPFDQWPIYQVSQAVQERVFAYTEEEFNASKEALFSGDISSKSRDFWFKTNNISDQGIGAQGLNTILSHAFSRYSGVIKKVENRNKKRLKKLSKKNQLKIEEGLEILEFKPDSAFNENGLLAQPPGINPNIYGYQAVTPFVFDPDNPGDVILPKQYEGYSRKPDDIIEKGPSRLDIPKGQPGYVPEHQRKNLKKKGRVRLYRRTPPKTKALASILAVLQIGKDWVLFDMRGLLRSVYMREAATPGQISAKDLLDTFTGCPVLNTRTGEFTFCYKLRSEGALHARKIYTKGETRTLLTSLTSENNTIALVTVDLGQRNPAAIMISRLSRKEELSEKDIQPVSRRLLPDRYLNELKRYRDAYDAFRQEVRDEAFTSLCPEHQEQVQQYEALTPEKAKNLVLKHFFGTHDPDLPWDDMTSNTHYIANLYLERGGDPSKVFFTRPLKKDSKSKKPRKPTKRTDASISRLPEIRPKMPEDARKAFEKAKWEIYTGHEKFPKLAKRVNQLCREIANWIEKEAKRLTLCDTVVVGIEDLSLPPKRGKGKFQETWQGFFRQKFENRWVIDTLKKAIQNRAHDKGKYVLGLAPYWTSQRCPACGFIHKSNRNGDHFKCLKCEALFHADSEVATWNLALVAVLGKGITNPDSKKPSGQKKTGTTRKKQIKGKNKGKETVNVPPTTQEVEDIIAFFEKDDETVRNPVYKPTGT

SEQ ID NO: 35 - protein, 769 aa, synthetic construct
MKKPNNIRRIREEHFEGLCFGKDVLTKAGKIYEKDGEEAAIDFLMGKDEEDPPNFKPPAKTTIVAQSRPFDQWPIYQVSQAVQERVFAYTEEEFNASKEALFSGDISSKSRDFWFKTNNISDQGIGAQGLNTILSHAFSRYSGVIKKVENRNKKRLKKLSKKNQLKIEEGLEILEFKPDSAFNENGLLAQPPGINPNIYGYQAVTPFVFDPDNPGDVILPKQYEGYSRKPDDIIEKGPSRLDIPKGQPGYVPEHQRKNLKKKGRVRLYRRTPPKTKALASILAVLQIGKDWVLFDMRGLLRSVYMREAATPGQISAKDLLDTFTGCPVLNTRTGEFTFCYKLRSEGALHARKIYTKGETRTLLTSLTSENNTIALVTVDLGQRNPAAIMISRLSRKEELSEKDIQPVSRRLLPDRYLNELKRYRDAYDAFRQEVRDEAFTSLCPEHQEQVQQYEALTPEKAKNLVLKHFFGTHDPDLPWDDMTSNTHYIANLYLERGGDPSKVFFTRPLKKDSKSKKPRKPTKRTDASISRLPEIRPKMPEDARKAFEKAKWEIYTGHEKFPKLAKRVNQLCREIANWIEKEAKRLTLCDTVVVGIEDLSLPPKRGKGKFQETWQGFFRQKFENRWVIDTLKKAIQNRAHDKGKYVLGLAPYWTSQRCPACGFIHKSNRNGDHFKCLKCEALFHADSEVATWNLALVAVLGKGITNPDSKKPSGQKKTGTTRKKQIKGKNKGKETVNVPPTTQEVEDIIAFFEKDDETVRNPVYKPTGT

SEQ ID NO: 36 - protein, 767 aa, synthetic construct
VIKTHFPAGRFRKDHQKTAGKKLKHEGEEACVEYLRNKVSDYPPNFKPPAKGTIVAQSRPFSEWPIVRASEAIQKYVYGLTVAELDVFSPGTSKPSHAEWFAKTGVENYGYRQVQGLNTIFQNTVNRFKGVLKKVENRNKKSLKRQEGANRRRVEEGLPEVPVTVESATDDEGRLLQPPGVNPSIYGYQGVAPRVCTDLQGFSGMSVDFAGYRRDPDAVLVESLPEGRLSIPKGERGYVPEWQRDPERNKFPLREGSRRQRKWYSNACHKPKPGRTSKYDPEALKKASAKDALLVSISIGEDWAIIDVRGLLRDARRRGFTPEEGLSLNSLLGLFTEYPVFDVQRGLITFTYKLGQVDVHSRKTVPTFRSRALLESLVAKEEIALVSVDLGQTNPASMKVSRVRAQEGALVAEPVHRMFLSDVLLGELSSYRKRMDAFEDAIRAQAFETMTPEQQAEITRVCDVSVEVARRRVCEKYSISPQDVPWGEMTGHSTFIVDAVLRKGGDESLVYFKNKEGETLKFRDLRISRMEGVRPRLTKDTRDALNKAVLDLKRAHPTFAKLAKQKLELARRCVNFIEREAKRYTQCERVVFVIEDLNVGFFHGKGKRDRGWDAFFTAKKENRWVIQALHKAFSDLGLHRGSYVIEVTPQRTSMTCPRCGHCDKGNRNGEKFVCLQCGATLHADLEVATDNIERVALTGKAMPKPPVRERSGDVQKAGTARKARKPLKPKQKTEPSVQEGSSDDGVDKSPGDASRNPVYNPSDTLSI

SEQ ID NO: 37 - protein, 763 aa, synthetic construct
MAKAKTLAALLRELLPGQHLAPHHRWVANKLLMTSGDAAAFVIGKSVSDPVRGSFRKDVITKAGRIFKKDGPDAAAAFLDGKWEDRPPNFQPPAKAAIVAISRSFDEWPIVKVSCAIQQYLYALPVQEFESSVPEARAQAHAAWFQDTGVDDCNFKSTQGLNAIFNHGKRTYEGVLKKAQNRNDKKNLRLERINAKRAEAGQAPLVAGPDESPTDDAGCLLHPPGINANIYCYQQVSPRPYEQSCGIQLPPEYAGYNRLSNVAIPPMPNRLDIPQGQPGYVPEHHRHGIKKFGRVRKRYGVVPGRNRDADGKRTRQVLTEAGAAAKARDSVLAVIRIGDDWTVVDLRGLLRNAQWRKLVPDGGITVQGLLDLFTGDPVIDPRRGVVTFIYKADSVGIHSEKVCRGKQSKNLLERLCAMPEKSSTRLDCARQAVALVSVDLGQRNPVAARFSRVSLAEGQLQAQLVSAQFLDDAMVAMIRSYREEYDRFESLVREQAKAALSPEQLSEIVRHEADSAESVKSCVCAKFGIDPAGLSWDKMTSGTWRIADHVQAAGGDVEWFFFKTCGKGKEIKTVRRSDFNVAKQFRLRLSPETRKDWNDAIWELKRGNPAYVSFSKRKSEFARRVVNDLVHRARRAVRCDEVVFAIEDLNISFFHGKGQRQMGWDAFFEVKQENRWFIQALHKAFVERATHKGGYVLEVAPARTSTTCPECRHCDPESRRGEQFCCIKCRHTCHADLEVATFNIEQVALTGVSLPKRLSSTLL

SEQ ID NO: 38 - protein, 761 aa, synthetic construct
MSKEKTPPSAYAILKAKHFPDLDFEKKHKMMAGRMFKNGASEQEVVQYLQGKGSESLMDVKPPAKSPILAQSRPFDEWEMVRTSRLIQETIFGIPKRGSIPKRDGLSETQFNELVASLEVGGKPMLNKQTRAIFYGLLGIKPPTFHAMAQNILIDLAINIRKGVLKKVDNLNEKNRKKVKRIRDAGEQDVMVPAEVTAHDDRGYLNHPPGVNPTIPGYQGVVIPFPEGFEGLPSGMTPVDWSHVLVDYLPHDRLSIPKGSPGYIPEWQRPLLNRHKGRRHRSWYANSLNKPRKSRTEEAKDRQNAGKRTALIEAERLKGVLPVLMRFKEDWLIIDARGLLRNARYRGVLPEGSTLGNLIDLFSDSPRVDTRRGICTFLYRKGRAYSTKPVKRKESKETLLKLTEKSTIALVSIDLGQTNPLTAKLSKVRQVDGCLVAEPVLRKLIDNASEDGKEIARYRVAHDLLRARILEDAIDLLGIYKDEVVRARSDTPDLCKERVCRFLGLDSQAIDWDRMTPYTDFIAQAFVAKGGDPKVVTIKPNGKPKMFRKDRSIKNMKGIRLDISKEASSAYREAQWAIQRESPDFQRLAVWQSQLTKRIVNQLVAWAKKCTQCDTVVLAFEDLNIGMMHGSGKWANGGWNALFLHKQENRWFMQAFHKALTELSAHKGIPTIEVLPHRTSITCTQCGHCHPGNRDGERFKCLKCEFLANTDLEIATDNIERVALTGLPMPKGERSSAKRKPGGTRKTKKSKHSGNSPLAAE

SEQ ID NO: 39 - protein, 746 aa, synthetic construct
MEKAGPTSPLSVLIHKNFEGCRFQIDHLKIAGRKLAREGEAAAIEYLLDKKCEGLPPNFQPPAKGNVIAQSRPFTEWAPYRASVAIQKYIYSLSVDERKVCDPGSSSDSHEKWFKQTGVQNYGYTHVQGLNLIFKHALARYDGVLKKVDNRNEKNRKKAERVNSFRREEGLPEEVFEEEKATDETGHLLQPPGVNHSIYCYQSVRPKPFNPRKPGGISLPEAYSGYSLKPQDELPIGSLDRLSIPPGQPGYVPEWQRSQLTTQKHRRKRSWYSAQKWKPRTGRTSTFDPDRLNCARAQGAILAVVRIHEDWVVFDVRGLLRNALWRELAGKGLTVRDLLDFFTGDPVVDTKRGVVTFTYKLGKVDVHSLRTVRGKRSKKVLEDLTLSSDVGLVTIDLGQTNVLAADYSKVTRSENGELLAVPLSKSFLPKHLLHEVTAYRTSYDQMEEGFRRKALLTLTEDQQVEVTLVRDFSVESSKTKLLQLGVDVTSLPWEKMSSNTTYISDQLLQQGADPASLFFDGERDGKPCRHKKKDRTWAYLVRPKVSPETRKALNEALWALKNTSPEFESLSKRKIQFSRRCMNYLLNEAKRISGCGQVVFVIEDLNVRVHHGRGKRAIGWDNFFKPKRENRWFMQALHKAASELAIHRGMHIIEACPARSSITCPKCGHCDPENRCSSDREKFLCVKCGAAFHADLEVATFNLRKVALTGTALPKSIDHSRDGLIPKGARNRKLKEPQANDEKACA

SEQ ID NO: 40 - protein, 735 aa, synthetic construct
MKEQSPLSSVLKSNFPGKKFLSADIRVAGRKLAQLGEAAAVEYLSPRQRDSVPNFRPPAFCTVVAKSRPFEEWPIYKASVLLQEQIYGMTGQEFEERCGSIPTSLSGLRQWASSVGLGAAMEGLHVQGMNLMVKNAINRYKGVLVKVENRNKKLVEANEAKNSSREERGLPPLRPPELGSAFGPDGRLVNPPGIDKSIRLYQGVSPVPVVKTTGRPTVHRLDIPAGEKGHVPLWQREAGLVKEGPRRRRMWYSNSNLKRSRKDRSAEASEARKADSVVVRVSVKEDWVDIDVRGLLRNVAWRGIERAGESTEDLLSLFSGDPVVDPSRDSVVFLYKEGVVDVLSKKVVGAGKSRKQLEKMVSEGPVALVSCDLGQTNYVAARVSVLDESLSPVRSFRVDPREFPSADGSQGVVGSLDRIRADSDRLEAKLLSEAEASLPEPVRAEIEFLRSERPSAVAGRLCLKLGIDPRSIPWEKMGSTTSFISEALSAKGSPLALHDGAPIKDSRFAHAARGRLSPESRKALNEALWERKSSSREYGVISRRKSEASRRMANAVLSESRRLTGLAVVAVNLEDLNMVSKFFHGRGKRAPGWAGFFTPKMENRWFIRSIHKAMCDLSKHRGITVIESRPERTSISCPECGHCDPENRSGERFSCKSCGVSLHADFEVATRNLERVALTGKPMPRRENLHSPEGATASRKTRKKPREATASTFLDLRSVLSSAENEGSGPAARAG

SEQ ID NO: 41 - protein, 725 aa, synthetic construct
MLPPSNKIGKSMSLKEFINKRNFKSSIIKQAGKILKKEGEEAVKKYLDDNYVEGYKKRDFPITAKCNIVASNRKIEDFDISKFSSFIQNYVFNLNKDNFEEFSKIKYNRKSFDELYKKIANEIGLEKPNYENIQGEIAVIRNAINIYNGVLKKVENRNKKIQEKNQSKDPPKLLSAFDDNGFLAERPGINETIYGYQSVRLRHLDVEKDKDIIVQLPDIYQKYNKKSTDKISVKKRLNKYNVDEYGKLISKRRKERINKDDAILCVSNFGDDWIIFDARGLLRQTYRYKLKKKGLCIKDLLNLFTGDPIINPTKTDLKEALSLSFKDGIINNRTLKVKNYKKCPELISELIRDKGKVAMISIDLGQTNPISYRLSKFTANNVAYIENGVISEDDIVKMKKWREKSDKLENLIKEEAIASLSDDEQREVRLYENDIADNTKKKILEKFNIREEDLDFSKMSNNTYFIRDCLKNKNIDESEFTFEKNGKKLDPTDACFAREYKNKLSELTRKKINEKIWEIKKNSKEYHKISIYKKETIRYIVNKLIKQSKEKSECDDIIVNIEKLQIGGNFFGGRGKRDPGWNNFFLPKEENRWFINACHKAFSELAPHKGIIVIESDPAYTSQTCPKCENCDKENRNGEKFKCKKCNYEANADIDVATENLEKIAKNGRRLIKNFDQLGERLPGAEMPGGARKRKPSKSLPKNGRGAGVGSEPELINQSPSQVIA

SEQ ID NO: 42 - protein, 718 aa, synthetic construct
VPDKKETPLVALCKKSFPGLRFKKHDSRQAGRILKSKGEGAAVAFLEGKGGTTQPNFKPPVKCNIVAMSRPLEEWPIYKASVVIQKYVYAQSYEEFKATDPGKSEAGLRAWLKATRVDTDGYFNVQGLNLIFQNARATYEGVLKKVENRNSKKVAKIEQRNEHRAERGLPLLTLDEPETALDETGHLRHRPGINCSVFGYQHMKLKPYVPGSIPGVTGYSRDPSTPIAACGVDRLEIPEGQPGYVPPWDRENLSVKKHRRKRASWARSRGGAIDDNMLLAVVRVADDWALLDLRGLLRNTQYRKLLDRSVPVTIESLLNLVTNDPTLSVVKKPGKPVRYTATLIYKQGVVPVVKAKVVKGSYVSKMLDDTTETFSLVGVDLGVNNLIAANALRIRPGKCVERLQAFTLPEQTVEDFFRFRKAYDKHQENLRLAAVRSLTAEQQAEVLALDTFGPEQAKMQVCGHLGLSVDEVPWDKVNSRSSILSDLAKERGVDDTLYMFPFFKGKGKKRKTEIRKRWDVNWAQHFRPQLTSETRKALNEAKWEAERNSSKYHQLSIRKKELSRHCVNYVIRTAEKRAQCGKVIVAVEDLHHSFRRGGKGSRKSGWGGFFAAKQEGRWLMDALFGAFCDLAVHRGYRVIKVDPYNTSRTCPECGHCDKANRDRVNREAFICVCCGYRGNADIDVAAYNIAMVAITGVSLRKAARASVASTPLESLAAE

SEQ ID NO: 43 - protein, 708 aa, synthetic construct
MSKTKELNDYQEALARRLPGVRHQKSVRRAARLVYDRQGEDAMVAFLDGKEVDEPYTLQPPAKCHILAVSRPIEEWPIARVTMAVQEHVYALPVHEVEKSRPETTEGSRSAWFKNSGVSNHGVTHAQTLNAILKNAYNVYNGVIKKVENRNAKKRDSLAAKNKSRERKGLPHFKADPPELATDEQGYLLQPPSPNSSVYLVQQHLRTPQIDLPSGYTGPVVDPRSPIPSLIPIDRLAIPPGQPGYVPLHDREKLTSNKHRRMKLPKSLRAQGALPVCFRVFDDWAVVDGRGLLRHAQYRRLAPKNVSIAELLELYTGDPVIDIKRNLMTFRFAEAVVEVTARKIVEKYHNKYLLKLTEPKGKPVREIGLVSIDLNVQRLIALAIYRVHQTGESQLALSPCLHREILPAKGLGDFDKYKSKFNQLTEEILTAAVQTLTSAQQEEYQRYVEESSHEAKADLCLKYSITPHELAWDKMTSSTQYISRWLRDHGWNASDFTQITKGRKKVERLWSDSRWAQELKPKLSNETRRKLEDAKHDLQRANPEWQRLAKRKQEYSRHLANTVLSMAREYTACETVVIAIENLPMKGGFVDGNGSRESGWDNFFTHKKENRWMIKDIHKALSDLAPNRGVHVLEVNPQYTSQTCPECGHRDKANRDPIQRERFCCTHCGAQRHADLEVATHNIAMVATTGKSLTGKSLAPQRLQEAAE

SEQ ID NO: 44 - protein, 848 aa, synthetic construct
MTPSFGYQMIIVTPIHHASGAWATLRLLFLNPKTSGVMLGMTKTKSAFALMREEVFPGLLFKSADLKMAGRKFAKEGREAAIEYLRGKDEERPANFKPPAKGDIIAQSRPFDQWPIVQVSQAIQKYIFGLTKAEFDATKTLLYGEGNHPTTESRRRWFEATGVPDFGFTSAQGLNAIFSSALARYEGVIQKVENRNEKRLKKLSEKNQRLVEEGHAVEAYVPETAFHTLESLKALSEKSLVPLDDLMDKIDRLAQPPGINPCLYGYQQVAPYIYDPENPRGVVLPDLYLGYCRKPDDPITACPNRLDIPKGQPGYIPEHQRGQLKKHGRVRRFRYTNPQAKARAKAQTAILAVLRIDEDWVVMDLRGLLRNVYFREVAAPGELTARTLLDTFTGCPVLNLRSNVVTFCYDIESKGALHAEYVRKGWATRNKLLDLTKDGQSVALLSVDLGQRHPVAVMISRLKRDDKGDLSEKSIQVVSRTFADQYVDKLKRYRVQYDALRKEIYDAALVSLPPEQQAEIRAYEAFAPGDAKANVLSVMFQGEVSPDELPWDKMNTNTHYISDLYLRRGGDPSRVFFVPQPSTPKKNAKKPPAPRKPVKRTDENVSHMPEFRPHLSNETREAFQKAKWTMERGNVRYAQLSRFLNQIVREANNWLVSEAKKLTQCQTVVWAIEDLHVPFFHGKGKYHETWDGFFRQKKEDRWFVNVFHKAISERAPNKGEYVMEVAPYRTSQRCPVCGFVDADNRHGDHFKCLRCGVELHADLEVATWNIALVAVQGHGIAGPPREQSCGGETAGTARKGKNIKKNKGLADAVTVEAQDSEGGSKKDAGTARNPVYIPSESQVNCPAP

SEQ ID NO: 45 - protein, 781 aa, synthetic construct
MKPKTPKPPKTPVAALIDKHFPGKRFRASYLKSVGKKLKNQGEDVAVRFLTGKDEERPPNFQPPAKSNIVAQSRPIEEWPIHKVSVAVQEYVYGLTVAEKEACSDAGESSSSHAAWFAKTGVENFGYTSVQGLNKIFPPTFNRFDGVIKKVENRNEKKRQKATRINEAKRNKGQSEDPPEAEVKATDDAGYLLQPPGINHSVYGYQSITLCPYTAEKFPTIKLPEEYAGYHSNPDAPIPAGVPDRLAIPEGQPGHVPEEHRAGLSTKKHRRVRQWYAMANWKPKPKRTSKPDYDRLAKARAQGALLIVIRIDEDWVVVDARGLLRNVRWRSLGKREITPNELLDLFTGDPVLDLKRGVVTFTYAEGVVNVCSRSTTKGKQTKVLLDAMTAPRDGKKRQIGMVAVDLGQTNPIAAEYSRVGKNAAGTLEATPLSRSTLPDELLREIALYRKAHDRLEAQLREEAVLKLTAEQQAENARYVETSEEGAKLALANLGVDTSTLPWDAMTGWSTCISDHLINHGGDTSAVFFQTIRKGTKKLETIKRKDSSWADIVRPRLTKETREALNDFLWELKRSHEGYEKLSKRLEELARRAVNHVVQEVKWLTQCQDIVIVIEDLNVRNFHGGGKRGGGWSNFFTVKKENRWFMQALHKAFSDLAAHRGIPVLEVYPARTSITCLGCGHCDPENRDGEAFVCQQCGATFHADLEVATRNIARVALTGEAMPKAPAREQPGGAKKRGTSRRRKLTEVAVKSAEPTIHQAKNQQLNGTSRDPVYKGSELPAL

SEQ ID NO: 46 - protein, 752 aa, synthetic construct
MSDNKMKKLSKEEKPLTPLQILIRKYIDKSQYPSGFKTTIIKQAGVRIKSVKSEQDEINLANWIISKYDPTYIKRDFNPSAKCQIIATSRSVADFDIVKMSNKVQEIFFASSHLDKNVFDIGKSKSDHDSWFERNNVDRGIYTYSNVQGMNLIFSNTKNTYLGVAVKAQNKFSSKMKRIQDINNFRITNHQSPLPIPDEIKIYDDAGFLLNPPGVNPNIFGYQSCLLKPLENKEIISKTSFPEYSRLPADMIEVNYKISNRLKFSNDQKGFIQFKDKLNLFKINSQELFSKRRRLSGQPILLVASFGDDWVVLDGRGLLRQVYYRGIAKPGSITISELLGFFTGDPIVDPIRGVVSLGFKPGVLSQETLKTTSARIFAEKLPNLVLNNNVGLMSIDLGQTNPVSYRLSEITSNMSVEHICSDFLSQDQISSIEKAKTSLDNLEEEIAIKAVDHLSDEDKINFANFSKLNLPEDTRQSLFEKYPELIGSKLDFGSMGSGTSYIADELIKFENKDAFYPSGKKKFDLSFSRDLRKKLSDETRKSYNDALFLEKRTNDKYLKNAKRRKQIVRTVANSLVSKIEELGLTPVINIENLAMSGGFFDGRGKREKGWDNFFKVKKENRWVMKDFHKAFSELSPHHGVIVIESPPYCTSVTCTKCNFCDKKNRNGHKFTCQRCGLDANADLDIATENLEKVAISGKRMPGSERSSDERKVAVARKAKSPKGKAIKGVKCTITDEPALLSANSQDCSQSTS

SEQ ID NO: 47 - protein, 747 aa, synthetic construct
MALSLAEVRERHFKGLRFRSSYLKRAGKILKKEGEAACVAYLTGKDEESPPNFKPPAKCDVVAQSRPFEEWPIVQASVAVQSYVYGLTKEAFEAFNPGTTKQSHEACLAATGIDTCGYSNVQGLNLIFRQAKNRYEGVITKVENRNKKAKKKLTRKNEWRQKNGHSELPEAPEELTFNDEGRLLQPPGINPSLYTYQQISPTPWSPKDSSILPPQYAGYERDPNAPIPFGVAKDRLTIASGCPGYIPEWMRTAGEKTNPRTQKKFMHPGLSTRKNKRMRLPRSVRSAPLGALLVTIHLGEDWLVLDVRGLLRNARWRGVAPKDISTQGLLNLFTGDPVIDTRRGVVTFTYKPETVGIHSRTWLYKGKQTKEVLEKLTQDQTVALVAIDLGQTNPVSAAASRVSRSGENLSIETVDRFFLPDELIKELRLYRMAHDRLEERIREESTLALTEAQQAEVRALEHVVRDDAKNKVCAAFNLDAASLPWDQMTSNTTYLSEAILAQGVSRDQVFFTPNPKKGSKEPVEVMRKDRAWVYAFKAKLSEETRKAKNEALWALKRASPDYARLSKRREELCRRSVNMVINRAKKRTQCQVVIPVLEDLNIGFFHGSGKRLPGWDNFFVAKKENRWLMNGLHKSFSDLAVHRGFYVFEVMPHRTSITCPACGHCDSENRDGEAFVCLSCKRTYHADLDVATHNLTQVAGTGLPMPEREHPGGTKKPGGSRKPESPQTHAPILHRTDYSESADRLGS

SEQ ID NO: 48 - protein, 702 aa, synthetic construct
MIKEQSELSKLIEKYYPGKKFYSNDLKQAGKHLKKSEHLTAKESEELTVEFLKSCKEKLYDFRPPAKALIISTSRPFEEWPIYKASESIQKYIYSLTKEELEKYNISTDKTSQENFFKESLIDNYGFANVSGLNLIFQHTKAIYDGVLKKVNNRNNKILKKYKRKIEEGIEIDSPELEKAIDESGHFINPPGINKNIYCYQQVSPTIFNSFKETKIICPFNYKRNPNDIIQKGVIDRLAIPFGEPGYIPDHQRDKVNKHKKRIRKYYKNNENKNKDAILAKINIGEDWVLFDLRGLLRNAYWRKLIPKQGITPQQLLDMFSGDPVIDPIKNNITFIYKESIIPIHSESIIKTKKSKELLEKLTKDEQIALVSIDLGQTNPVAARFSRLSSDLKPEHVSSSFLPDELKNEICRYREKSDLLEIEIKNKAIKMLSQEQQDEIKLVNDISSEELKNSVCKKYNIDNSKIPWDKMNGFTTFIADEFINNGGDKSLVYFTAKDKKSKKEKLVKLSDKKIANSFKPKISKETREILNKITWDEKISSNEYKKLSKRKLEFARRATNYLINQAKKATRLNNVVLVVEDLNSKFFHGSGKREDGWDNFFIPKKENRWFIQALHKSLTDVSIHRGINVIEVRPERTSITCPKCGCCDKENRKGEDFKCIKCDSVYHADLEVATFNIEKVAITGESMPKPDCERLGGEESIG

SEQ ID NO: 49 - protein, 661 aa, synthetic construct
VAFLDGKEVDEPYTLQPPAKCHILAVSRPIEEWPIARVTMAVQEHVYALPVHEVEKSRPETTEGSRSAWFKNSGVSNHGVTHAQTLNAILKNAYNVYNGVIKKVENRNAKKRDSLAAKNKSRERKGLPHFKADPPELATDEQGYLLQPPSPNSSVYLVQQHLRTPQIDLPSGYTGPVVDPRSPIPSLIPIDRLAIPPGQPGYVPLHDREKLTSNKHRRMKLPKSLRAQGALPVCFRVFDDWAVVDGRGLLRHAQYRRLAPKNVSIAELLELYTGDPVIDIKRNLMTFRFAEAVVEVTARKIVEKYHNKYLLKLTEPKGKPVREIGLVSIDLNVQRLIALAIYRVHQTGESQLALSPCLHREILPAKGLGDFDKYKSKFNQLTEEILTAAVQTLTSAQQEEYQRYVEESSHEAKADLCLKYSITPHELAWDKMTSSTQYISRWLRDHGWNASDFTQITKGRKKVERLWSDSRWAQELKPKLSNETRRKLEDAKHDLQRANPEWQRLAKRKQEYSRHLANTVLSMAREYTACETVVIAIENLPMKGGFVDGNGSRESGWDNFFTHKKENRWMIKDIHKALSDLAPNRGVHVLEVNPQYTSQTCPECGHRDKANRDPIQRERFCCTHCGAQRHADLEVATHNIAMVATTGKSLTGKSLAPQRLQ

SEQ ID NO: 50 - protein, 491 aa, synthetic construct
VLLSDRIQYTDPSAPIPAMTVVDRRKIKKGEPGYVPPFMRKNLSTNKHRRMRLSRGQKEACALPVGLRLPDGKDGWDFIIFDGRALLRACRRLRLEVTSMDDVLDKFTGDPRIQLSPAGETIVTCMLKPQHTGVIQQKLITGKMKDRLVQLTAEAPIAMLTVDLGEHNLVACGAYTVGQRRGKLQSERLEAFLLPEKVLADFEGYRRDSDEHSETLRHEALKALSKRQQREVLDMLRTGADQARESLCYKYGLDLQALPWDKMSSNSTFIAQHLMSLGFGESATHVRYRPKRKASERTILKYDSRFAAEEKIKLTDETRRAWNEAIWECQRASQEFRCLSVRKLQLARAAVNWTLTQAKQRSRCPRVVVVVEDLNVRFMHGGGKRQEGWAGFFKARSEKRWFIQALHKAYTELPTNRGIHVMEVNPARTSITCTKCGYCDPENRYGEDFHCRNPKCKVRGGHVANADLDIATENLARVALSGPMPKAPKLK

SEQ ID NO: 51 - protein, 531 aa, synthetic construct
LEIPEGEPGHVPWFQRMDIPEGQIGHVNKIQRFNFVHGKNSGKVKFSDKTGRVKRYHHSKYKDATKPYKFLEESKKVSALDSILAIITIGDDWVVFDIRGLYRNVFYRELAQKGLTAVQLLDLFTGDPVIDPKKGIITFSYKEGVVPVFSQKIVSRFKSRDTLEKLTSQGPVALLSVDLGQNEPVAARVCSLKNINDKIALDNSCRIPFLDDYKKQIKDYRDSLDELEIKIRLEAINSLDVNQQVEIRDLDVFSADRAKASTVDMFDIDPNLISWDSMSDARFSTQISDLYLKNGGDESRVYFEINNKRIKRSDYNISQLVRPKLSDSTRKNLNDSIWKLKRTSEEYLKLSKRKLELSRAVVNYTIRQSKLLSGINDIVIILEDLDVKKKFNGRGIRDIGWDNFFSSRKENRWFIPAFHKSFSELSSNRGLCVIEVNPAWTSATCPDCGFCSKENRDGINFTCRKCGVSYHADIDVATLNIARVAVLGKPMSGPADRERLGGTKKPRVARSRKDMKRKDISNGTVEVMVTA

SEQ ID NO: 52 - protein, 767 aa, synthetic construct
MSEITDLLKANFKGKTFKSADMRMAGRILKKSGAQAVIKYLSDKGAVDPPDFRPPAKCNIIAQSRPFDEWPICKASMAIQQHIYGLTKNEFDESSPGTSSASHEQWFAKTGVDTHGFTHVQGLNLIFQHAKKRYEGVIKKVENYNEKERKKFEGINERRSKEGMPLLEPRLRTAFGDDGKFAEKPGVNPSIYLYQQTSPRPYDKTKHPYVHAPFELKEITTIPTQDDRLKIPFGAPGHVPEKHRSQLSMAKHKRRRAWYALSQNKPRPPKDGSKGRRSVRDLADLKAASLADAIPLVSRVGFDWVVIDGRGLLRNLRWRKLAHEGMTVEEMLGFFSGDPVIDPRRNVATFIYKAEHATVKSRKPIGGAKRAREELLKATASSDGVIRQVGLISVDLGQTNPVAYEISRMHQANGELVAEHLEYGLLNDEQVNSIQRYRAAWDSMNESFRQKAIESLSMEAQDEIMQASTGAAKRTREAVLTMFGPNATLPWSRMSSNTTCISDALIEVGKEEETNFVTSNGPRKRTDAQWAAYLRPRVNPETRALLNQAVWDLMKRSDEYERLSKRKLEMARQCVNFVVARAEKLTQCNNIGIVLENLVVRNFHGSGRRESGWEGFFEPKRENRWFMQVLHKAFSDLAQHRGVMVFEVHPAYSSQTCPACRYVDPKNRSSEDRERFKCLKCGRSFNADREVATFNIREIARTGVGLPKPDCERSRGVQTTGTARNPGRSLKSNKNPSEPKRVLQSKTRKKITSTETQNEPLATDLKT

SEQ ID NO: 53 - protein, 760 aa, synthetic construct
MTPKTESPLSALCKKHFPGKRFRTNYLKDAGKILKKHGEDAVVAFLSDKQEDEPANFCPPAKVHILAQSRPFEDWPINLASKAIQTYVYGLTADERKTCEPGTSKESHDRWFKETGVDHHGFTSVQGLNLIFKHTLNRYDGVIKKVETRNEKRRSSVVRINEKKAAEGLPLIAAEAEETAFGEDGRLLQPPGVNHSIYCFQQVSPQPYSSKKHPQVVLPHAVQGVDPDAPIPVGRPNRLDIPKGQPGYVPEWQRPHLSMKCKRVRMWYARANWRRKPGRRSVLNEARLKEASAKGALPIVLVIGDDWLVMDARGLLRSVFWRRVAKPGLSLSELLNVTPTGLFSGDPVIDPKRGLVTFTSKLGVVAVHSRKPTRGKKSKDLLLKMTKPTDDGMPRHVGMVAIDLGQTNPVAAEYSRVVQSDAGTLKQEPVSRGVLPDDLLKDVARYRRAYDLTEESIRQEAIALLSEGHRAEVTKLDQTTANETKRLLVDRGVSESLPWEKMSSNTTYISDCLVALGKTDDVFFVPKAKKGKKETGIAVKRKDHGWSKLLRPRTSPEARKALNENQWAVKRASPEYERLSRRKLELGRRCVNHIIQETKRWTQCEDIVVVLEDLNVGFFHGSGKRPDGWDNFFVSKRENRWFIQVLHKAFGDLATHRGTHVIEVHPARTSITCIKCGHCDAGNRDGESFVCLASACGDRRHADLEVATRNVARVAITGERMPPSEQARDVQKAGGARKRKPSARNVKSSYPAVEPAPASP

SEQ ID NO: 54 - protein, 733 aa, synthetic construct
QAVIKYLSDKGAVDPPDFRPPAKCNIIAQSRPFDEWPICKASMAIQQHIYGLTKNEFDESSPGTSSASHEQWFAKTGVDTHGFTHVQGLNLIFQHAKKRYEGVIKKVENYNEKERKKFEGINERRSKEGMPLLEPRLRTAFGDDGKFAEKPGVNPSIYLYQQTSPRPYDKTKHPYVHAPFELKEITTIPTQDDRLKIPFGAPGHVPEKHRSQLSMAKHKRRRAWYALSQNKPRPPKDGSKGRRSVRDLADLKAASLADAIPLVSRVGFDWVVIDGRGLLRNLRWRKLAHEGMTVEEMLGFFSGDPVIDPRRNVATFIYKAEHATVKSRKPIGGAKRAREELLKATASSDGVIRQVGLISVDLGQTNPVAYEISRMHQANGELVAEHLEYGLLNDEQVNSIQRYRAAWDSMNESFRQKAIESLSMEAQDEIMQASTGAAKRTREAVLTMFGPNATLPWSRMSSNTTCISDALIEVGKEEETNFVTSNGPRKRTDAQWAAYLRPRVNPETRALLNQAVWDLMKRSDEYERLSKRKLEMARQCVNFVVARAEKLTQCNNIGIVLENLVVRNFHGSGRRESGWEGFFEPKRENRWFMQVLHKAFSDLAQHRGVMVFEVHPAYSSQTCPACRYVDPKNRSSEDRERFKCLKCGRSFNADREVATFNIREIARTGVGLPKPDCERSRDVQTPGTARKSGRSLKSQDNLSEPKRVLQSKTRKKITSTETQNEPLATDLKT

SEQ ID NO: 55 - protein, 479 aa, synthetic construct
IPSFGYLDRLKIAKGQPGYIPEWQRETINPSKKVRRYWATNHEKIRNAIPLVVFIGDDWVIIDGRGLLRDARRRKLADKNTTIEQLLEMVSNDPVIDSTRGIATLSYVEGVVPVRSFIPIGEKKGREYLEKSTQKESVTLLSVDIGQINPVSCGVYKVSNGCSKIDFLDKFFLDKKHLDAIQKYRTLQDSLEASIVNEALDEIDPSFKKEYQNINSQTSNDVKKSLCTEYNIDPEAISWQDITAHSTLISDYLIDNNITNDVYRTVNKAKYKTNDFGWYKKFSAKLSKEAREALNEKIWELKIASSKYKKLSVRKKEIARTIANDCVKRAETYGDNVVVAMESLTKNNKVMSGRGKRDPGWHNLGQAKVENRWFIQAISSAFEDKATHHGTPVLKVNPAYTSQTCPSCGHCSKDNRSSKDRTIFVCKSCGEKFNADLDVATYNIAHVAFSGKKLSPPSEKSSATKKPRSARKSKKSRKS

SEQ ID NO: 56 - protein, 452 aa, synthetic construct
SPIEKLLNGLLVKITFGNDWIICDARGLLDNVQKGIIHKSYFTNKSSLVDLIDLFTCNPIVNYKNNVVTFCYKEGVVDVKSFTPIKSGPKTQENLIKKLKYSRFQNEKDACVLGVGVDVGVTNPFAINGFKMPVDESSEWVMLNEPLFTIETSQAFREEIMAYQQRTDEMNDQFNQQSIDLLPPEYKVEFDNLPEDINEVAKYNLLHTLNIPNNFLWDKMSNTTQFISDYLIQIGRGTETEKTITTKKGKEKILTIRDVNWFNTFKPKISEETGKARTEIKRDLQKNSDQFQKLAKSREQSCRTWVNNVTEEAKIKSGCPLIIFVIEALVKDNRVFSGKGHRAIGWHNFGKQKNERRWWVQAIHKAFQEQGVNHGYPVILCPPQYTSQTCPKCNHVDRDNRSGEKFKCLKYGWIGNADLDVGAYNIARVAITGKALSKPLEQKKIKKAKNKT

SEQ ID NO: 57 - protein, 425 aa, synthetic construct
LLDNVQKGIIHKSYFTNKSSLVDLIDLFTCNPIVNYKNNVVTFCYKEGVVDVKSFTPIKSGPKTQENLIKKLKYSRFQNEKDACVLGVGVDVGVTNPFAINGFKMPVDESSEWVMLNEPLFTIETSQAFREEIMAYQQRTDEMNDQFNQQSIDLLPPEYKVEFDNLPEDINEVAKYNLLHTLNIPNNFLWDKMSNTTQFISDYLIQIGRGTETEKTITTKKGKEKILTIRDVNWFNTFKPKISEETGKARTEIKRDLQKNSDQFQKLAKSREQSCRTWVNNVTEEAKIKSGCPLIIFVIEALVKDNRVFSGKGHRAIGWHNFGKQKNERRWWVQAIHKAFQEQGVNHGYPVILCPPQYTSQTCPKCNHVDRDNRSGEKFKCLKYGWIGNADLDVGAYNIARVAITGKALSKPLEQKKIKKAKNKT

SEQ ID NO: 58 - protein, 735 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKEF

SEQ ID NO: 59 - protein, 492 aa, synthetic construct; identical to SEQ ID NO: 9

SEQ ID NO: 60 - protein, 478 aa, synthetic construct
MKDYIRKTLSLRILRPYYGEEIEKEIAAAKKKSQAEGGDGALDNKFWDRLKAEHPEIISSREFYDLLDAIQRETTLYYNRAISKLYHSLIVEREQVSTAKALSAGPYHEFREKFNAYISLGLREKIQSNFRRKELARYQVALPTAKSDTFPIPIYKGFDKNGKGGFKVREIENGDFVIDLPLMAYHRVGGKAGREYIELDRPPAVLNVPVILSTSRRRANKTWFRDEGTDAEIRRVMAGEYKVSWVEILQRKRFGKPYGGWYVNFTIKYQPRDYGLDPKVKGGIDIGLSSPLVCAVTNSLARLTIRDNDLVAFNRKAMARRRTLLRQNRYKRSGHGSANKLKPIEALTEKNELYRKAIMRRWAREAADFFRQHRAATVNMEDLTGIKDREDYFSQMLRCYWNYSQLQTMLENKLKEYGIAVKYIEPKDTSKTCHSCGHVNEYFDFNYRSAHKFPMFKCEKCGVECGADYNAARNIAQA

SEQ ID NO: 61 - protein, 477 aa, synthetic construct
VKISKTLSLRIIRPYYTPEVESAIKAEKDKREAQGQTRNLDAKFFNELKKKHPQIILSGEFYSLLFEMQRQLTSIYNRAMSSLYHKIIVEGEKTSTSKALSDIGYDECKSVFPSYIALGLRQKIQSNFRRKELKGFRMAVPTAKSDKFPIPIYKQVDDGKGGFKISENKEGDFIVELPLVEYTAEDVKTAKGKFTKINISKPPKIKNIPVILSTLRRKQSGQWFSDEGTNAEIRRVISGEYKVSWIEVVRRTRFGKHDDWFLNIVIKYDKTEDGLDPEVVGGIDVGVSTPLVCAVNNSLDRYFVKSSDIIAFKKRAMARRRTLLRQNRFKRSGHGSKSKLEPITILTEKNERFKKSIMQRWAKEVAEFFKGERASVVQMEELSGLKEKDNFFGSYLRMYWNYGQLQQIIENKLKEYGIKVNYVSPKDTSKKCHSCGYINEFFTFEFRQKNNFPLFKCKKCGVECNADYNAAKNIAIA

SEQ ID NO: 62 - protein, 472 aa, synthetic construct
VPITKTISLRILRPYYPPEIEAKIKAEKEKRKENGDTGSLNSSYYRELKKEYPSIIINDEFFPLLSEMQRNITSIYNRTISHLYHRLIIKKESISTAKALSEGPYRDFKSTFNSYIALGLRQKVQSNFRKKDLMAFKIALPTAKSDKFPIPIYMQTNFKIKESPDSDFIIELPLVEYIAKETKGKNKMFTKVEILSPPKVKNIPVILSTRRRKESGQWFSDEGTNAEIRRIISGEYKVSWIEIVKRTRFGKHDWFVNMVISFEESQEGLDPDVIGGIDIGVSKPLICAINNSLDRYIVKGDDIIAFNRRALSRRRSLLRRNRLKRSGHGSRNKLEPITVLTEKNERFKKSIMQRWAKEVAEFFKSKRASIVQMEELTGIKEREDFFSKTLRMYWNYGQLQKTVENKLREYGIEVRYASPKDTSRRCHSCGHINDYFTFEFRQQNNFPLFKCMNCGIECSADYNAARNIAIAR

SEQ ID NO: 63 - protein, 500 aa, synthetic construct
MEVQKTVMKTLSLRILRPLYSQEIEKEIKEEKERRKQAGGTGELDGGFYKKLEKKHSEMFSFDRLNLLLNQLQREIAKVYNHAISELYIATIAQGNKSNKHYISSIVYNRAYGYFYNAYIALGICSKVEANFRSNELLTQQSALPTAKSDNFPIVLHKQKGAEGEDGGFRISTEGSDLIFEIPIPFYEYNGENRKEPYKWVKKGGQKPVLKLILSTFRRQRNKGWAKDEGTDAEIRKVTEGKYQVSQIEINRGKKLGEHQKWFANFSIEQPIYERKPNRSIVGGLDVGIRSPLVCAINNSFSRYSVDSNDVFKFSKQVFAFRRRLLSKNSLKRKGHGAAHKLEPITEMTEKNDKFRKKIIERWAKEVTNFFVKNQVGIVQIEDLSTMKDREDHFFNQYLRGFWPYYQMQTLIENKLKEYGIEVKRVQAKYTSQLCSNPNCRYWNNYFNFEYRKVNKFPKFKCEKCNLEISADYNAARNLSTPDIEKFVAKATKGINLPEK

SEQ ID NO: 64 - protein, 507 aa, synthetic construct
MEEAKTVSKTLSLRILRPLYSAEIEKEIKEEKERRKQGGKSGELDSGFYKKLEKKHTQMFGWDKLNLMLSQLQRQIARVFNQSISELYIETVIQGKKSNKHYTSKIVYNRAYSVFYNAYLALGITSKVEANFRSTELLMQKSSLPTAKSDNFPILLHKQKGVEGEEGGFKISADGNDLIFEIPIPFYEYDSANKKEPFKWIKKGGQKPTIKLILSTFRRQRNKGWAKDEGTDAEIRKVIEGKYQVSHIEINRGKKLGDHQKWFVNFTIEQPIYERKLDKNIIGGIDVGIKSPLVCAVNNSFARYSVDSNDVLKFSKQAFAFRRRLLSKNSLKRSGHGSKNKLDPITRMTEKNDRFRKKIIERWAKEVTNFFIKNQVGTVQIEDLSTMKDRQDNFFNQYLRGFWPYYQMQNLIENKLKEYGIETKRIKARYTSQLCSNPSCRHWNSYFSFDHRKTNNFPKFKCEKCALEISADYNAARNISTPDIEKFVAKATKGINLPDKNENVILE

SEQ ID NO: 65 - protein, 723 aa, synthetic construct
MERQKVPQIRKIVRVVPLRILRPKYSDVIENALKKFKEKGDDTNTNDFWRAIRDRDTEFFRKELNFSEDEINQLERDTLFRVGLDNRVLFSYFDFLQEKLMKDYNKIISKLFINRQSKSSFENDLTDEEVEELIEKDVTPFYGAYIGKGIKSVIKSNLGGKFIKSVKIDRETKKVTKLTAINIGLMGLPVAKSDTFPIKIIKTNPDYITFQKSTKENLQKIEDYETGIEYGDLLVQITIPWFKNENKDFSLIKTKEAIEYYKLNGVGKKDLLNINLVLTTYHIRKKKSWQIDGSSQSLVREMANGELEEKWKSFFDTFIKKYGDEGKSALVKRRVNKKSRAKGEKGRELNLDERIKRLYDSIKAKSFPSEINLIPENYKWKLHFSIEIPPMVNDIDSNLYGGIDFGEQNIATLCVKNIEKDDYDFLTIYGNDLLKHAQASYARRRIMRVQDEYKARGHGKSRKTKAQEDYSERMQKLRQERLVKQISDFFLWRNKFHMAVCSLRYEDLNTLYKGESVKAKRMRQFINKQQLFNGIERKLKDYNSEIYVNSRYPHYTSRLCSKCGKLNLYFDFLKFRTKNIIIRKNPDGSEIKYMPFFICEFCGWKQAGDKNASANIADKDYQDKLNKEKEFCNIRKPKSKKEDIGEENEEERDYSRRFNRNSFIYNSLKKDNKLNQEKLFDEWKNQLKRKIDGRNKFEPKEYKDRFSYLFAYYQEIIKNESES

SEQ ID NO: 66 - protein, 517 aa, synthetic construct
MVPTELITKTLQLRVIRPLYFEEIEKELAELKEQKEKEFEETNSLLLESKKIDAKSLKKLKRKARSSAAVEFWKIAKEKYPDILTKPEMEFIFSEMQKMMARFYNKSMTNIFIEMNNDEKVNPLSLISKASTEANQVIKCSSISSGLNRKIAGSINKTKFKQVRDGLISLPTARTETFPISFYKSTANKDEIPISKINLPSEEEADLTITLPFPFFEIKKEKKGQKAYSYFNIIEKSGRSNNKIDLLLSTHRRQRRKGWKEEGGTSAEIRRLMEGEFDKEWEIYLGEAEKSEKAKNDLIKNMTRGKLSKDIKEQLEDIQVKYFSDNNVESWNDLSKEQKQELSKLRKKKVEELKDWKHVKEILKTRAKIGWVELKRGKRQRDRNKWFVNITITRPPFINKELDDTKFGGIDLGVKVPFVCAVHGSPARLIIKENEILQFNKMVSARNRQITKDSEQRKGRGKKNKFIKKEIFNERNELFRKKIIERWANQIVKFFEDQKCATVQIENLESFDRTSYK

SEQ ID NO: 67 - protein, 481 aa, synthetic construct
MKSDTKDKKIIIHQTKTLSLRIVKPQSIPMEEFTDLVRYHQMIIFPVYNNGAIDLYKKLFKAKIQKGNEARAIKYFMNKIVYAPIANTVKNSYIALGYSTKMQSSFSGKRLWDLRFGEATPPTIKADFPLPFYNQSGFKVSSENGEFIIGIPFGQYTKKTVSDIEKKTSFAWDKFTLEDTTKKTLIELLLSTKTRKMNEGWKNNEGTEAEIKRVMDGTYQVTSLEILQRDDSWFVNFNIAYDSLKKQPDRDKIAGIHMGITRPLTAVIYNNKYRALSIYPNTVMHLTQKQLARIKEQRTNSKYATGGHGRNAKVTGTDTLSEAYRQRRKKIIEDWIASIVKFAINNEIGTIYLEDISNTNSFFAAREQKLIYLEDISNTNSFLSTYKYPISAISDTLQHKLEEKAIQVIRKKAYYVNQICSLCGHYNKGFTYQFRRKNKFPKMKCQGCLEATSTEFNAAANVANPDYEKLLIKHGLLQLKK

SEQ ID NO: 68 - protein, 358 aa, synthetic construct
MSTITRQVRLSPTPEQSRLLMAHCQQYISTVNVLVAAFDSEVLTGKVSTKDFRAALPSAVKNQALRDAQSVFKRSVELGCLPVLKKPHCQWNNQNWRVEGDQLILPICKDGKTQQERFRCAAVALEGKAGILRIKKKRGKWIADLTVTQEDAPESSGSAIMGVDLGIKVPAVAHIGGKGTRFFGNGRSQRSMRRRFYARRKTLQKAKKLRAVRKSKGKEARWMKTINHQLSRQIVNHAHALGVGTIKIEALQGIRKGTTRKSRGAAARKNNRMTNTWSFSQLTLFITYKAQRQGITVEQVDPAYTSQDCPACRARNGAQDRTYVCSECGWRGHRDTVGAINISRRAGLSGHRRGATGA

SEQ ID NO: 69 - protein, 507 aa, synthetic construct
MIAQKTIKIKLNPTKEQIIKLNSIIEEYIKVSNFTAKKIAEIQESFTDSGLTQGTCSECGKEKTYRKYHLLKKDNKLFCITCYKRKYSQFTLQKVEFQNKTGLRNVAKLPKTYYTNAIRFASDTFSGFDEIIKKKQNRLNSIQNRLNFWKELLYNPSNRNEIKIKVVKYAPKTDTREHPHYYSEAEIKGRIKRLEKQLKKFKMPKYPEFTSETISLQRELYSWKNPDELKISSITDKNESMNYYGKEYLKRYIDLINSQTPQILLEKENNSFYLCFPITKNIEMPKIDDTFEPVGIDWGITRNIAVVSILDSKTKKPKFVKFYSAGYILGKRKHYKSLRKHFGQKKRQDKINKLGTKEDRFIDSNIHKLAFLIVKEIRNHSNKPIILMENITDNREEAEKSMRQNILLHSVKSRLQNYIAYKALWNNIPTNLVKPEHTSQICNRCGHQDRENRPKGSKLFKCVKCNYMSNADFNASINIARKFYIGEYEPFYKDNEKMKSGVNSISM

SEQ ID NO: 70 - protein, 534 aa, synthetic construct
LKLSEQENITTGVKFKLKLDKETSEGLNDYFDEYGKAINFAIKVIQKELAEDRFAGKVRLDENKKPLLNEDGKKIWDFPNEFCSCGKQVNRYVNGKSLCQECYKNKFTEYGIRKRMYSAKGRKAEQDINIKNSTNKISKTHFNYAIREAFILDKSIKKQRKERFRRLREMKKKLQEFIEIRDGNKILCPKIEKQRVERYIHPSWINKEKKLEDFRGYSMSNVLGKIKILDRNIKREEKSLKEKGQINFKARRLMLDKSVKFLNDNKISFTISKNLPKEYELDLPEKEKRLNWLKEKIKIIKNQKPKYAYLLRKDDNFYLQYTLETEFNLKEDYSGIVGIDRGVSHIAVYTFVHNNGKNERPLFLNSSEILRLKNLQKERDRFLRRKHNKKRKKSNMRNIEKKIQLILHNYSKQIVDFAKNKNAFIVFEKLEKPKKNRSKMSKKSQYKLSQFTFKKLSDLVDYKAKREGIKVLYISPEYTSKECSHCGEKVNTQRPFNGNSSLFKCNKCGVELNADYNASINIAKKGLNILNSTN

SEQ ID NO: 71 - protein, 537 aa, synthetic construct
MEESIITGVKFKLRIDKETTKKLNEYFDEYGKAINFAVKIIQKELADDRFAGKAKLDQNKNPILDENGKKIYEFPDEFCSCGKQVNKYVNNKPFCQECYKIRFTENGIRKRMYSAKGRKAEHKINILNSTNKISKTHFNYAIREAFILDKSIKKQRKKRNERLRESKKRLQQFIDMRDGKREICPTIKGQKVDRFIHPSWITKDKKLEDFRGYTLSIINSKIKILDRNIKREEKSLKEKGQIIFKAKRLMLDKSIRFVGDRKVLFTISKTLPKEYELDLPSKEKRLNWLKEKIEIIKNQKPKYAYLLRKNIESEKKPNYEYYLQYTLEIKPELKDFYDGAIGIDRGINHIAVCTFISNDGKVTPPKFFSSGEILRLKNLQKERDRFLLRKHNKNRKKGNMRVIENKINLILHRYSKQIVDMAKKLNASIVFEELGRIGKSRTKMKKSQRYKLSLFIFKKLSDLVDYKSRREGIRVTYVPPEYTSKECSHCGEKVNTQRPFNGNYSLFKCNKCGIQLNSDYNASINIAKKGLKIPNST

SEQ ID NO: 72 - protein, 540 aa, synthetic construct
LWTIVIGDFIEMPKQDLVTTGIKFKLDVDKETRKKLDDYFDEYGKAINFAVKIIQKNLKEDRFAGKIALGEDKKPLLDKDGKKIYNYPNESCSCGNQVRRYVNAKPFCVDCYKLKFTENGIRKRMYSARGRKADSDINIKNSTNKISKTHFNYAIREGFILDKSLKKQRSKRIKKLLELKRKLQEFIDIRQGQMVLCPKIKNQRVDKFIHPSWLKRDKKLEEFRGYSLSVVEGKIKIFNRNILREEDSLRQRGHVNFKANRIMLDKSVRFLDGGKVNFNLNKGLPKEYLLDLPKKENKLSWLNEKISLIKLQKPKYAYLLRREGSFFIQYTIENVPKTFSDYLGAIGIDRGISHIAVCTFVSKNGVNKAPVFFSSGEILKLKSLQKQRDLFLRGKHNKIRKKSNMRNIDNKINLILHKYSRNIVNLAKSEKAFIVFEKLEKIKKSRFKMSKSLQYKLSQFTFKKLSDLVEYKAKIEGIKVDYVPPEYTSKECSHCGEKVDTQRPFNGNSSLFKCNKCRVQLNADYNASINIAKKSLNISN

SEQ ID NO: 73 - protein, 542 aa, synthetic construct
MSKTTISVKLKIIDLSSEKKEFLDNYFNEYAKATTFCQLRIRRLLRNTHWLGKKEKSSKKWIFESGICDLCGENKELVNEDRNSGEPAKICKRCYNGRYGNQMIRKLFVSTKKREVQENMDIRRVAKLNNTHYHRIPEEAFDMIKAADTAEKRRKKNVEYDKKRQMEFIEMFNDEKKRAARPKKPNERETRYVHISKLESPSKGYTLNGIKRKIDGMGKKIERAEKGLSRKKIFGYQGNRIKLDSNWVRFDLAESEITIPSLFKEMKLRITGPTNVHSKSGQIYFAEWFERINKQPNNYCYLIRKTSSNGKYEYYLQYTYEAEVEANKEYAGCLGVDIGCSKLAAAVYYDSKNKKAQKPIEIFTNPIKKIKMRREKLIKLLSRVKVRHRRRKLMQLSKTEPIIDYTCHKTARKIVEMANTAKAFISMENLETGIKQKQQARETKKQKFYRNMFLFRKLSKLIEYKALLKGIKIVYVKPDYTSQTCSSCGADKEKTERPSQAIFRCLNPTCRYYQRDINADFNAAVNIAKKALNNTEVVTTLL

SEQ ID NO: 74 - protein, 564 aa, synthetic construct
MARAKNQPYQKLTTTTGIKFKLDLSEEEGKRFDEYFSEYAKAVNFCAKVIYQLRKNLKFAGKKELAAKEWKFEISNCDFCNKQKEIYYKNIANGQKVCKGCHRTNFSDNAIRKKMIPVKGRKVESKFNIHNTTKKISGTHRHWAFEDAADIIESMDKQRKEKQKRLRREKRKLSYFFELFGDPAKRYELPKVGKQRVPRYLHKIIDKDSLTKKRGYSLSYIKNKIKISERNIERDEKSLRKASPIAFGARKIKMSKLDPKRAFDLENNVFKIPGKVIKGQYKFFGTNVANEHGKKFYKDRISKILAGKPKYFYLLRKKVAESDGNPIFEYYVQWSIDTETPAITSYDNILGIDAGITNLATTVLIPKNLSAEHCSHCGNNHVKPIFTKFFSGKELKAIKIKSRKQKYFLRGKHNKLVKIKRIRPIEQKVDGYCHVVSKQIVEMAKERNSCIALEKLEKPKKSKFRQRRREKYAVSMFVFKKLATFIKYKAAREGIEIIPVEPEGTSYTCSHCKNAQNNQRPYFKPNSKKSWTSMFKCGKCGIELNSDYNAAFNIAQKALNMTSA

SEQ ID NO: 75 - protein, 610 aa, synthetic construct
MDEKHFFCSYCNKELKISKNLINKISKGSIREDEAVSKAISIHNKKEHSLILGIKFKLFIENKLDKKKLNEYFDNYSKAVTFAARIFDKIRSPYKFIGLKDKNTKKWTFPKAKCVFCLEEKEVAYANEKDNSKICTECYLKEFGENGIRKKIYSTRGRKVEPKYNIFNSTKELSSTHYNYAIRDAFQLLDALKKQRQKKLKSIFNQKLRLKEFEDIFSDPQKRIELSLKPHQREKRYIHLSKSGQESINRGYTLRFVRGKIKSLTRNIEREEKSLRKKTPIHFKGNRLMIFPAGIKFDFASNKVKISISKNLPNEFNFSGTNVKNEHGKSFFKSRIELIKTQKPKYAYVLRKIKREYSKLRNYEIEKIRLENPNADLCDFYLQYTIETESRNNEEINGIIGIDRGITNLACLVLLKKGDKKPSGVKFYKGNKILGMKIAYRKHLYLLKGKRNKLRKQRQIRAIEPKINLILHQISKDIVKIAKEKNFAIALEQLEKPKKARFAQRKKEKYKLALFTFKNLSTLIEYKSKREGIPVIYVPPEKTSQMCSHCAINGDEHVDTQRPYKKPNAQKPSYSLFKCNKCGIELNADYNAAFNIAQKGLKTLMLNHSH

SEQ ID NO: 76 - protein, 369 aa, synthetic construct
MLQTLLVKLDPSKEQYKMLYETMERFNEACNQIAETVFAIHSANKIEVQKTVYYPIREKFGLSAQLTILAIRKVCEAYKRDKSIKPEFRLDGALVYDQRVLSWKGLDKVSLVTLQGRQIIPIKFGDYQKARMDRIRGQADLILVKGVFYLCVVVEVSEESPYDPKGVLGVDLGIKNLAVDSDGEVHSGEQTTNTRERLDSLKARLQSKGTKSAKRHLKKLSGRMAKFSKDVNHCISKKLVAKAKGTLMSIALEDLQGIRDRVTVRKAQRRNLHTWNFGLLRMFVDYKAKIAGVPLVFVDPRNTSRTCPSCGHVAKANRPTRDEFRCVSCGFAGAADHIAAMNIAFRAEVSQPIVTRFFVQSQAPSFRVG

SEQ ID NO: 77 - protein, 552 aa, synthetic construct
MDEEPDSAEPNLAPISVKLKLVKLDGEKLAALNDYFNEYAKAVNFCELKMQKIRKNLVNIRGTYLKEKKAWINQTGECCICKKIDELRCEDKNPDINGKICKKCYNGRYGNQMIRKLFVSTNKRAVPKSLDIRKVARLHNTHYHRIPPEAADIIKAIETAERKRRNRILFDERRYNELKDALENEEKRVARPKKPKEREVRYVPISKKDTPSKGYTMNALVRKVSGMAKKIERAKRNLNKRKKIEYLGRRILLDKNWVRFDFDKSEISIPTMKEFFGEMRFEITGPSNVMSPNGREYFTKWFDRIKAQPDNYCYLLRKESEDETDFYLQYTWRPDAHPKKDYTGCLGIDIGGSKLASAVYFDADKNRAKQPIQIFSNPIGKWKTKRQKVIKVLSKAAVRHKTKKLESLRNIEPRIDVHCHRIARKIVGMALAANAFISMENLEGGIREKQKAKETKKQKFSRNMFVFRKLSKLIEYKALMEGVKVVYIVPDYTSQLCSSCGTNNTKRPKQAIFMCQNTECRYFGKNINADFNAAINIAKKALNRKDIVRELS

SEQ ID NO: 78 - protein, 534 aa, synthetic construct
MEKNNSEQTSITTGIKFKLKLDKETKEKLNNYFDEYGKAINFAVRIIQMQLNDDRLAGKYKRDEKGKPILGEDGKKILEIPNDFCSCGNQVNHYVNGVSFCQECYKKRFSENGIRKRMYSAKGRKAEQDINIKNSTNKISKTHFNYAIREAFNLDKSIKKQREKRFKKLKDMKRKLQEFLEIRDGKRVICPKIEKQKVERYIHPSWINKEKKLEEFRGYSLSIVNSKIKSFDRNIQREEKSLKEKGQINFKAQRLMLDKSVKFLKDNKVSFTISKELPKTFELDLPKKEKKLNWLNEKLEIIKNQKPKYAYLLRKENNIFLQYTLDSIPEIHSEYSGAVGIDRGVSHIAVYTFLDKDGKNERPFFLSSSGILRLKNLQKERDKFLRKKHNKIRKKGNMRNIEQKINLILHEYSKQIVNFAKDKNAFIVFELLEKPKKSRERMSKKIQYKLSQFTFKKLSDLVDYKAKREGIKVIYVEPAYTSKDCSHCGERVNTQRPFNGNFSLFKCNKCGIVLNSDYNASLNIARKGLNISAN

SEQ ID NO: 79 - protein, 577 aa, synthetic construct
MAEEKFFFCEKCNKDIKIPKNYINKQGAEEKARAKHEHRVHALILGIKFKIYPKKEDISKLNDYFDEYAKAVTFTAKIVDKLKAPFLFAGKRDKDTSKKKWVFPVDKCSFCKEKTEINYRTKQGKNICNSCYLTEFGEQGLLEKIYATKGRKVSSSFNLFNSTKKLTGTHNNYVVKESLQLLDALKKQRSKRLKKLSNTRRKLKQFEEMFEKEDKRFQLPLKEKQRELRFIHVSQKDRATEFKGYTMNKIKSKIKVLRRNIEREQRSLNRKSPVFFRGTRIRLSPSVQFDDKDNKIKLTLSKELPKEYSFSGLNVANEHGRKFFAEKLKLIKENKSKYAYLLRRQVNKNNKKPIYDYYLQYTVEFLPNIITNYNGILGIDRGINTLACIVLLENKKEKPSFVKFFSGKGILNLKNKRRKQLYFLKGVHNKYRKQQKIRPIEPRIDQILHDISKQIIDLAKEKRVAISLEQLEKPQKPKFRQSRKAKYKLSQFNFKTLSNYIDYKAKKEGIRVIYIAPEMTSQNCSRCAMKNDLHVNTQRPYKNTSSLFKCNKCGVELNADYNAAFNIAQKGLKILNS

SEQ ID NO: 80 - protein, 613 aa, synthetic construct
MISLKLKLLPDEEQKKLLDEMFWKWASICTRVGFGRADKEDLKPPKDAEGVWFSLTQLNQANTDINDLREAMKHQKHRLEYEKNRLEAQRDDTQDALKNPDRREISTKRKDLFRPKASVEKGFLKLKYHQERYWVRRLKEINKLIERKTKTLIKIEKGRIKFKATRITLHQGSFKIRFGDKPAFLIKALSGKNQIDAPFVVVPEQPICGSVVNSKKYLDEITTNFLAYSVNAMLFGLSRSEEMLLKAKRPEKIKKKEEKLAKKQSAFENKKKELQKLLGRELTQQEEAIIEETRNQFFQDFEVKITKQYSELLSKIANELKQKNDFLKVNKYPILLRKPLKKAKSKKINNLSPSEWKYYLQFGVKPLLKQKSRRKSRNVLGIDRGLKHLLAVTVLEPDKKTFVWNKLYPNPITGWKWRRRKLLRSLKRLKRRIKSQKHETIHENQTRKKLKSLQGRIDDLLHNISRKIVETAKEYDAVIVVEDLQSMRQHGRSKGNRLKTLNYALSLFDYANVMQLIKYKAGIEGIQIYDVKPAGTSQNCAYCLLAQRDSHEYKRSQENSKIGVCLNPNCQNHKKQIDADLNAARVIASCYALKINDSQPFGTRKRFKKRTTN

SEQ ID NO: 81 - protein, 615 aa, synthetic construct
METLSLKLKLNPSKEQLLVLDKMFWKWASICTRLGLKKAEMSDLEPPKDAEGVWFSKTQLNQANTDVNDLRKAMQHQGKRIEYELDKVENRRNEIQEMLEKPDRRDISPNRKDLFRPKAAVEKGYLKLKYHKLGYWSKELKTANKLIERKRKTLAKIDAGKMKFKPTRISLHTNSFRIKFGEEPKIALSTTSKHEKIELPLITSLQRPLKTSCAKKSKTYLDAAILNFLAYSTNAALFGLSRSEEMLLKAKKPEKIEKRDRKLATKRESFDKKLKTLEKLLERKLSEKEKSVFKRKQTEFFDKFCITLDETYVEALHRIAEELVSKNKYLEIKKYPVLLRKPESRLRSKKLKNLKPEDWTYYIQFGFQPLLDTPKPIKTKTVLGIDRGVRHLLAVSIFDPRTKTFTFNRLYSNPIVDWKWRRRKLLRSIKRLKRRLKSEKHVHLHENQFKAKLRSLEGRIEDHFHNLSKEIVDLAKENNSVIVVENLGGMRQHGRGRGKWLKALNYALSHFDYAKVMQLIKYKAELAGVFVYDVAPAGTSINCAYCLLNDKDASNYTRGKVINGKKNTKIGECKTCKKEFDADLNAARVIALCYEKRLNDPQPFGTRKQFKPKKP

SEQ ID NO: 82 - protein, 775 aa, synthetic construct
MKALKLQLIPTRKQYKILDEMFWKWASLANRVSQKGESKETLAPKKDIQKIQFNATQLNQIEKDIKDLRGAMKEQQKQKERLLLQIQERRSTISEMLNDDNNKERDPHRPLNFRPKGWRKFHTSKHWVGELSKILRQEDRVKKTIERIVAGKISFKPKRIGIWSSNYKINFFKRKISINPLNSKGFELTLMTEPTQDLIGKNGGKSVLNNKRYLDDSIKSLLMFALHSRFFGLNNTDTYLLGGKINPSLVKYYKKNQDMGEFGREIVEKFERKLKQEINEQQKKIIMSQIKEQYSNRDSAFNKDYLGLINEFSEVFNQRKSERAEYLLDSFEDKIKQIKQEIGESLNISDWDFLIDEAKKAYGYEEGFTEYVYSKRYLEILNKIVKAVLITDIYFDLRKYPILLRKPLDKIKKISNLKPDEWSYYIQFGYDSINPVQLMSTDKFLGIDRGLTHLLAYSVFDKEKKEFIINQLEPNPIMGWKWKLRKVKRSLQHLERRIRAQKMVKLPENQMKKKLKSIEPKIEVHYHNISRKIVNLAKDYNASIVVESLEGGGLKQHGRKKNARNRSLNYALSLFDYGKIASLIKYKADLEGVPMYEVLPAYTSQQCAKCVLEKGSFVDPEIIGYVEDIGIKGSLLDSLFEGTELSSIQVLKKIKNKIELSARDNHNKEINLILKYNFKGLVIVRGQDKEEIAEHPIKEINGKFAILDFVYKRGKEKVGKKGNQKVRYTGNKKVGYCSKHGQVDADLNASRVIALCKYLDINDPILFGEQRKSFK

SEQ ID NO: 83 - protein, 777 aa, synthetic construct
MVTRAIKLKLDPTKNQYKLLNEMFWKWASLANRFSQKGASKETLAPKDGTQKIQFNATQLNQIKKDVDDLRGAMEKQGKQKERLLIQIQERLLTISEILRDDSKKEKDPHRPQNFRPFGWRRFHTSAYWSSEASKLTRQVDRVRRTIERIKAGKINFKPKRIGLWSSTYKINFLKKKINISPLKSKSFELDLITEPQQKIIGKEGGKSVANSKKYLDDSIKSLLIFAIKSRLFGLNNKDKPLFENIITPNLVRYHKKGQEQENFKKEVIKKFENKLKKEISQKQKEIIFSQIERQYENRDATFSEDYLRAISEFSEIFNQRKKERAKELLNSFNEKIRQLKKEVNGNISEEDLKILEVEAEKAYNYENGFIEWEYSEQFLGVLEKIARAVLISDNYFDLKKYPILIRKPTNKSKKITNLKPEEWDYYIQFGYGLINSPMKIETKNFMGIDRGLTHLLAYSIFDRDSEKFTINQLELNPIKGWKWKLRKVKRSLQHLERRMRAQKGVKLPENQMKKRLKSIEPKIESYYHNLSRKIVNLAKANNASIVVESLEGGGLKQHGRKKNSRHRALNYALSLFDYGKIASLIKYKSDLEGVPMYEVLPAYTSQQCAKCVLKKGSFVEPEIIGYIEEIGFKENLLTLLFEDTGLSSVQVLKKSKNKMTLSARDKEGKMVDLVLKYNFKGLVISQEKKKEEIVEFPIKEIDGKFAVLDSAYKRGKERISKKGNQKLVYTGNKKVGYCSVHGQVDADLNASRVIALCKYLGINEPIVFGEQRKSFK

SEQ ID NO: 84 - protein, 610 aa, synthetic construct
LDLITEPIQPHKSSSLRSKEFLEYQISDFLNFSLHSLFFGLASNEGPLVDFKIYDKIVIPKPEERFPKKESEEGKKLDSFDKRVEEYYSDKLEKKIERKLNTEEKNVIDREKTRIWGEVNKLEEIRSIIDEINEIKKQKHISEKSKLLGEKWKKVNNIQETLLSQEYVSLISNLSDELTNKKKELLAKKYSKFDDKIKKIKEDYGLEFDENTIKKEGEKAFLNPDKFSKYQFSSSYLKLIGEIARSLITYKGFLDLNKYPIIFRKPINKVKKIHNLEPDEWKYYIQFGYEQINNPKLETENILGIDRGLTHILAYSVFEPRSSKFILNKLEPNPIEGWKWKLRKLRRSIQNLERRWRAQDNVKLPENQMKKNLRSIEDKVENLYHNLSRKIVDLAKEKNACIVFEKLEGQGMKQHGRKKSDRLRGLNYKLSLFDYGKIAKLIKYKAEIEGIPIYRIDSAYTSQNCAKCVLESRRFAQPEEISCLDDFKEGDNLDKRILEGTGLVEAKIYKKLLKEKKEDFEIEEDIAMFDTKKVIKENKEKTVILDYVYTRRKEIIGTNHKKNIKGIAKYTGNTKIGYCMKHGQVDADLNASRTIALCKNFDINNPEIWK

SEQ ID NO: 85 - protein, 632 aa, synthetic construct
MSDESLVSSEDKLAIKIKIVPNAEQAKMLDEMFKKWSSICNRISRGKEDIETLRPDEGKELQFNSTQLNSATMDVSDLKKAMARQGERLEAEVSKLRGRYETIDASLRDPSRRHTNPQKPSSFYPSDWDISGRLTPRFHTARHYSTELRKLKAKEDKMLKTINKIKNGKIVFKPKRITLWPSSVNMAFKGSRLLLKPFANGFEMELPIVISPQKTADGKSQKASAEYMRNALLGLAGYSINQLLFGMNRSQKMLANAKKPEKVEKFLEQMKNKDANFDKKIKALEGKWLLDRKLKESEKSSIAVVRTKFFKSGKVELNEDYLKLLKHMANEILERDGFVNLNKYPILSRKPMKRYKQKNIDNLKPNMWKYYIQFGYEPIFERKASGKPKNIMGIDRGLTHLLAVAVFSPDQQKFLFNHLESNPIMHWKWKLRKIRRSIQHMERRIRAEKNKHIHEAQLKKRLGSIEEKTEQHYHIVSSKIINWAIEYEAAIVLESLSHMKQRGGKKSVRTRALNYALSLFDYEKVARLITYKARIRGIPVYDVLPGMTSKTCATCLLNGSQGAYVRGLETTKAAGKATKRKNMKIGKCMVCNSSENSMIDADLNAARVIAICKYKNLNDPQPAGSRKVFKRF

SEQ ID NO: 86 - protein, 625 aa, synthetic construct
MLALKLKIMPTEKQAEILDAMFWKWASICSRIAKMKKKVSVKENKKELSKKIPSNSDIWFSKTQLCQAEVDVGDHKKALKNFEKRQESLLDELKYKVKAINEVINDESKREIDPNNPSKFRIKDSTKKGNLNSPKFFTLKKWQKILQENEKRIKKKESTIEKLKRGNIFFNPTKISLHEEEYSINFGSSKLLLNCFYKYNKKSGINSDQLENKFNEFQNGLNIICSPLQPIRGSSKRSFEFIRNSIINFLMYSLYAKLFGIPRSVKALMKSNKDENKLKLEEKLKKKKSSFNKTVKEFEKMIGRKLSDNESKILNDESKKFFEIIKSNNKYIPSEEYLKLLKDISEEIYNSNIDFKPYKYSILIRKPLSKFKSKKLYNLKPTDYKYYLQLSYEPFSKQLIATKTILGIDRGLKHLLAVSVFDPSQNKFVYNKLIKNPVFKWKKRYHDLKRSIRNRERRIRALTGVHIHENQLIKKLKSMKNKINVLYHNVSKNIVDLAKKYESTIVLERLENLKQHGRSKGKRYKKLNYVLSNFDYKKIESLISYKAKKEGVPVSNINPKYTSKTCAKCLLEVNQLSELKNEYNRDSKNSKIGICNIHGQIDADLNAARVIALCYSKNLNEPHFK

SEQ ID NO: 87 - protein, 517 aa, synthetic construct
VINLFGYKFALYPNKTQEELLNKHLGECGWLYNKAIEQNEYYKADSNIEEAQKKFELLPDKNSDEAKVLRGNISKDNYVYRTLVKKKKSEINVQIRKAVVLRPAETIRNLAKVKKKGLSVGRLKFIPIREWDVLPFKQSDQIRLEENYLILEPYGRLKFKMHRPLLGKPKTFCIKRTATDRWTISFSTEYDDSNMRKNDGGQVGIDVGLKTHLRLSNENPDEDPRYPNPKIWKRYDRRLTILQRRISKSKKLGKNRTRLRLRLSRLWEKIRNSRADLIQNETYEILSENKLIAIEDLNVKGMQEKKDKKGRKGRTRAQEKGLHRSISDAAFSEFRRVLEYKAKRFGSEVKPVSAIDSSKECHNCGNKKGMPLESRIYECPKCGLKIDRDLNSAKVILARATGVRPGSNARADTKISATAGASVQTEGTVSEDFRQQMETSDQKPMQGEGSKEPPMNPEHKSSGRGSKHVNIGCKNKVGLYNEDENSRSTEKQIMDENRSTTEDMVEIGALHSPVLTT

SEQ ID NO: 88 - protein, 410 aa, synthetic construct
MIASIDYEAVSQALIVFEFKAKGKDSQYQAIDEAIRSYRFIRNSCLRYWMDNKKVGKYDLNKYCKVLAKQYPFANKLNSQARQSAAECSWSAISRFYDNCKRKVSGKKGFPKFKKHARSVEYKTSGWKLSENRKAITFTDKNGIGKLKLKGTYDLHFSQLEDMKRVRLVRRADGYYVQFCISVDVKVETEPTGKAIGLDVGIKYFLADSSGNTIENPQFYRKAEKKLNRANRRKSKKYIRGVKPQSKNYHKARCRYARKHLRVSRQRKEYCKRVAYCVIHSNDVVAYEDLNVKGMVKNRHLAKSISDVAWSTFRHWLEYFAIKYGKLTIPVAPHNTSQNCSNCDKKVPKSLSTRTHICHHCGYSEDRDVNAAKNILKKALSTVGQTGSLKLGEIEPLLVLEQSCTRKFDL

SEQ ID NO: 89 - protein, 486 aa, synthetic construct
LAEENTLHLTLAMSLPLNDLPENRTRSELWRRQWLPQKKLSLLLGVNQSVRKAAADCLRWFEPYQELLWWEPTDPDGKKLLDKEGRPIKRTAGHMRVLRKLEEIAPFRGYQLGSAVKNGLRHKVADLLLSYAKRKLDPQFTDKTSYPSIGDQFPIVWTGAFVCYEQSITGQLYLYLPLFPRGSHQEDITNNYDPDRGPALQVFGEKEIARLSRSTSGLLLPLQFDKWGEATFIRGENNPPTWKATHRRSDKKWLSEVLLREKDFQPKRVELLVRNGRIFVNVACEIPTKPLLEVENFMGVSFGLEHLVTVVVINRDGNVVHQRQEPARRYEKTYFARLERLRRRGGPFSQELETFHYRQVAQIVEEALRFKSVPAVEQVGNIPKGRYNPRLNLRLSYWPFGKLADLTSYKAVKEGLPKPYSVYSATAKMLCSTCGAANKEGDQPISLKGPTVYCGNCGTRHNTGFNTALNLARRAQELFVKGVVAR

SEQ ID NO: 90 - protein, 602 aa, synthetic construct
MSQSLLKWHDMAGRDKDASRSLQKSAVEGVLLHLTASHRVALEMLEKSVSQTVAVTMEAAQQRLVIVLEDDPTKATSRKRVISADLQFTREEFGSLPNWAQKLASTCPEIATKYADKHINSIRIAWGVAKESTNGDAVEQKLQWQIRLLDVTMFLQQLVLQLADKALLEQIPSSIRGGIGQEVAQQVTSHIQLLDSGTVLKAELPTISDRNSELARKQWEDAIQTVCTYALPFSRERARILDPGKYAAEDPRGDRLINIDPMWARVLKGPTVKSLPLLFVSGSSIRIVKLTLPRKHAAGHKHTFTATYLVLPVSREWINSLPGTVQEKVQWWKKPDVLATQELLVGKGALKKSANTLVIPISAGKKRFFNHILPALQRGFPLQWQRIVGRSYRRPATHRKWFAQLTIGYTNPSSLPEMALGIHFGMKDILWWALADKQGNILKDGSIPGNSILDFSLQEKGKIERQQKAGKNVAGKKYGKSLLNATYRVVNGVLEFSKGISAEHASQPIGLGLETIRFVDKASGSSPVNARHSNWNYGQLSGIFANKAGPAGFSVTEITLKKAQRDLSDAEQARVLAIEATKRFASRIKRLATKRKDDTLFV

SEQ ID NO: 91 - protein, 494 aa, synthetic construct
VEPVEKERFYYRTYTFRLDGQPRTQNLTTQSGWGLLTKAVLDNTKHYWEIVHHARIANQPIVFENPVIDEQGNPKLNKLGQPRFWKRPISDIVNQLRALFENQNPYQLGSSLIQGTYWDVAENLASWYALNKEYLAGTATWGEPSFPEPHPLTEINQWMPLTFSSGKVVRLLKNASGRYFIGLPILGENNPCYRMRTIEKLIPCDGKGRVTSGSLILFPLVGIYAQQHRRMTDICESIRTEKGKLAWAQVSIDYVREVDKRRRMRRTRKSQGWIQGPWQEVFILRLVLAHKAPKLYKPRCFAGISLGPKTLASCVILDQDERVVEKQQWSGSELLSLIHQGEERLRSLREQSKPTWNAAYRKQLKSLINTQVFTIVTFLRERGAAVRLESIARVRKSTPAPPVNFLLSHWAYRQITERLKDLAIRNGMPLTHSNGSYGVRFTCSQCGATNQGIKDPTKYKVDIESETFLCSICSHREIAAVNTATNLAKQLLDE

SEQ ID NO: 92 - protein, 526 aa, synthetic construct
MNDTETSETLTSHRTVCAHLHVVGETGSLPRLVEAALAELITLNGRATQALLSLAKNGLVLRRDKEENLIAAELTLPCRKNKYADVAAKAGEPILATRINNKGKLVTKKWYGEGNSYHIVRFTPETGMFTVRVFDRYAFDEELLHLHSEVVFGSDLPKGIKAKTDSLPANFLQAVFTSFLELPFQGFPDIVVKPAMKQAAEQLLSYVQLEAGENQQAEYPDTNERDPELRLVEWQKSLHELSVRTEPFEFVRARDIDYYAETDRRGNRFVNITPEWTKFAESPFARRLPLKIPPEFCILLRRKTEGHAKIPNRIYLGLQIFDGVTPDSTLGVLATAEDGKLFWWHDHLDEFSNLEGKPEPKLKNKPQLLMVSLEYDREQRFEESVGGDRKICLVTLKETRNFRRGWNGRILGIHFQHNPVITWALMDHDAEVLEKGFIEGNAFLGKALDKQALNEYLQKGGKWVGDRSFGNKLKGITHTLASLIVRLAREKDAWIALEEISWVQKQSADSVANHEIVEQPHHSLTR

SEQ ID NO: 93 - protein, 649 aa, synthetic construct
MNDTETSETLTSHRTVCAHLHVVGETGSLPRLVEAALAELITLNGRATQALLSLAKNGLVLRRDKEENLIAAELTLPCRKNKYADVAAKAGEPILATRINNKGKLVTKKWYGEGNSYHIVRFTPETGMFTVRVFDRYAFDEELLHLHSEVVFGSDLPKGIKAKTDSLPANFLQAVFTSFLELPFQGFPDIVVKPAMKQAAEQLLSYVQLEAGENQQAEYPDTNERDPELRLVEWQKSLHELSVRTEPFEFVRARDIDYYAETDRRGNRFVNITPEWTKFAESPFARRLPLKIPPEFCILLRRKTEGHAKIPNRIYLGLQIFDGVTPDSTLGVLATAEDGKLFWWHDHLDEFSNLEGKPEPKLKNKPQLLMVSLEYDREQRFEESVGGDRKICLVTLKETRNFRRGRHGHTRTDRLPAGNTLWRADFATSAEVAAPKWNGRILGIHFQHNPVITWALMDHDAEVLEKGFIEGNAFLGKALDKQALNEYLQKGGKWVGDRSFGNKLKGITHTLASLIVRLAREKDAWIALEEISWVQKQSADSVANRRFSMWNYSRLATLIEWLGTDIATRDCGTAAPLAHKVSDYLTHFTCPECGACRKAGQKKEIADTVRAGDILTCRKCGFSGPIPDNFIAEFVAKKALERMLKKKPV

SEQ ID NO: 94 - protein, 414 aa, synthetic construct
MAKRNFGEKSEALYRAVRFEVRPSKEELSILLAVSEVLRMLFNSALAERQQVFTEFIASLYAELKSASVPEEISEIRKKLREAYKEHSISLFDQINALTARRVEDEAFASVTRNWQEETLDALDGAYKSFLSLRRKGDYDAHSPRSRDSGFFQKIPGRSGFKIGEGRIALSCGAGRKLSFPIPDYQQGRLAETTKLKKFELYRDQPNLAKSGRFWISVVYELPKPEATTCQSEQVAFVALGASSIGVVSQRGEEVIALWRSDKHWVPKIEAVEERMKRRVKGSRGWLRLLNSGKRRMHMISSRQHVQDEREIVDYLVRNHGSHFVVTELVVRSKEGKLADSSKPERGGSLGLNWAAQNTGSLSRLVRQLEEKVKEHGGSVRKHKLTLTEAPPARGAENKLWMARKLRESFLKEV

SEQ ID NO: 95 - protein, 413 aa, synthetic construct
LAKNDEKELLYQSVKFEIYPDESKIRVLTRVSNILVLVWNSALGERRARFELYIAPLYEELKKFPRKSAESNALRQKIREGYKEHIPTFFDQLKKLLTPMRKEDPALLGSVPRAYQEETLNTLNGSFVSFMTLRRNNDMDAKPPKGRAEDRFHEISGRSGFKIDGSEFVLSTKEQKLRFPIPNYQLEKLKEAKQIKKFTLYQSRDRRFWISIAYEIELPDQRPFNPEEVIYIAFGASSIGVISPEGEKVIDFWRPDKHWKPKIKEVENRMRSCKKGSRAWKKRAAARRKMYAMTQRQQKLNHREIVASLLRLGFHFVVTEYTVRSKPGKLADGSNPKRGGAPQGFNWSAQNTGSFGEFILWLKQKVKEQGGTVQTFRLVLGQSERPEKRGRDNKIEMVRLLREKYLESQTIVV

SEQ ID NO: 96 - protein, 449 aa, synthetic construct
MAKGKKKEGKPLYRAVRFEIFPTSDQITLFLRVSKNLQQVWNEAWQERQSCYEQFFGSIYERIGQAKKRAQEAGFSEVWENEAKKGLNKKLRQQEISMQLVSEKESLLQELSIAFQEHGVTLYDQINGLTARRIIGEFALIPRNWQEETLDSLDGSFKSFLALRKNGDPDAKPPRQRVSENSFYKIPGRSGFKVSNGQIYLSFGKIGQTLTSVIPEFQLKRLETAIKLKKFELCRDERDMAKPGRFWISVAYEIPKPEKVPVVSKQITYLAIGASRLGVVSPKGEFCLNLPRSDYHWKPQINALQERLEGVVKGSRKWKKRMAACTRMFAKLGHQQKQHGQYEVVKKLLRHGVHFVVTELKVRSKPGALADASKSDRKGSPTGPNWSAQNTGNIARLIQKLTDKASEHGGTVIKRNPPLLSLEERQLPDAQRKIFIAKKLREEFLADQK

SEQ ID NO: 97 - protein, 711 aa, synthetic construct
MAKREKKDDVVLRGTKMRIYPTDRQVTLMDMWRRRCISLWNLLLNLETAAYGAKNTRSKLGWRSIWARVVEENHAKALIVYQHGKCKKDGSFVLKRDGTVKHPPRERFPGDRKILLGLFDALRHTLDKGAKCKCNVNQPYALTRAWLDETGHGARTADIIAWLKDFKGECDCTAISTAAKYCPAPPTAELLTKIKRAAPADDLPVDQAILLDLFGALRGGLKQKECDHTHARTVAYFEKHELAGRAEDILAWLIAHGGTCDCKIVEEAANHCPGPRLFIWEHELAMIMARLKAEPRTEWIGDLPSHAAQTVVKDLVKALQTMLKERAKAAAGDESARKTGFPKFKKQAYAAGSVYFPNTTMFFDVAAGRVQLPNGCGSMRCEIPRQLVAELLERNLKPGLVIGAQLGLLGGRIWRQGDRWYLSCQWERPQPTLLPKTGRTAGVKIAASIVFTTYDNRGQTKEYPMPPADKKLTAVHLVAGKQNSRALEAQKEKEKKLKARKERLRLGKLEKGHDPNALKPLKRPRVRRSKLFYKSAARLAACEAIERDRRDGFLHRVTNEIVHKFDAVSVQKMSVAPMMRRQKQKEKQIESKKNEAKKEDNGAAKKPRNLKPVRKLLRHVAMARGRQFLEYKYNDLRGPGSVLIADRLEPEVQECSRCGTKNPQMKDGRRLLRCIGVLPDGTDCDAVLPRNRNAARNAEKRLRKHREAHNA

SEQ ID NO: 98 - protein, 574 aa, synthetic construct
MNEVLPIPAVGEDAADTIMRGSKMRIYPSVRQAATMDLWRRRCIQLWNLLLELEQAAYSGENRRTQIGWRSIWATVVEDSHAEAVRVAREGKKRKDGTFRKAPSGKEIPPLDPAMLAKIQRQMNGAVDVDPKTGEVTPAQPRLFMWEHELQKIMARLKQAPRTHWIDDLPSHAAQSVVKDLIKALQAMLRERKKRASGIGGRDTGFPKFKKNRYAAGSVYFANTQLRFEAKRGKAGDPDAVRGEFARVKLPNGVGWMECRMPRHINAAHAYAQATLMGGRIWRQGENWYLSCQWKMPKPAPLPRAGRTAAIKIAAAIPITTVDNRGQTREYAMPPIDRERIAAHAAAGRAQSRALEARKRRAKKREAYAKKRHAKKLERGIAAKPPGRARIKLSPGFYAAAAKLAKLEAEDANAREAWLHEITTQIVRNFDVIAVPRMEVAKLMKKPEPPEEKEEQVKAPWQGKRRSLKAARVMMRRTAMALIQTTLKYKAVDLRGPQAYEEIAPLDVTAAACSGCGVLKPEWKMARAKGREIMRCQEPLPGGKTCNTVLTYTRNSARVIGRELAVRLAERQKA

SEQ ID NO: 99 - protein, 400 aa, synthetic construct
MTTQKTYNFCFYDQRFFELSKEAGEVYSRSLEEFWKIYDETGVWLSKFDLQKHMRNKLERKLLHSDSFLGAMQQVHANLASWKQAKKVVPDACPPRKPKFLQAILFKKSQIKYKNGFLRLTLGTEKEFLYLKWDINIPLPIYGSVTYSKTRGWKINLCLETEVEQKNLSENKYLSIDLGVKRVATIFDGENTITLSGKKFMGLMHYRNKLNGKTQSRLSHKKKGSNNYKKIQRAKRKTTDRLLNIQKEMLHKYSSFIVNYAIRNDIGNIIIGDNSSTHDSPNMRGKTNQKISQNPEQKLKNYIKYKFESISGRVDIVPEPYTSRKCPHCKNIKKSSPKGRTYKCKKCGFIFDRDGVGAINIYNENVSFGQIISPGRIRSLTEPIGMKFHNEIYFKSYVAA

SEQ ID NO: 100 - protein, 743 aa, synthetic construct
MSVRSFQARVECDKQTMEHLWRTHKVFNERLPEIIKILFKMKRGECGQNDKQKSLYKSISQSILEANAQNADYLLNSVSIKGWKPGTAKKYRNASFTWADDAAKLSSQGIHVYDKKQVLGDLPGMMSQMVCRQSVEAISGHIELTKKWEKEHNEWLKEKEKWESEDEHKKYLDLREKFEQFEQSIGGKITKRRGRWHLYLKWLSDNPDFAAWRGNKAVINPLSEKAQIRINKAKPNKKNSVERDEFFKANPEMKALDNLHGYYERNFVRRRKTKKNPDGFDHKPTFTLPHPTIHPRWFVFNKPKTNPEGYRKLILPKKAGDLGSLEMRLLTGEKNKGNYPDDWISVKFKADPRLSLIRPVKGRRVVRKGKEQGQTKETDSYEFFDKHLKKWRPAKLSGVKLIFPDKTPKAAYLYFTCDIPDEPLTETAKKIQWLETGDVTKKGKKRKKKVLPHGLVSCAVDLSMRRGTTGFATLCRYENGKIHILRSRNLWVGYKEGKGCHPYRWTEGPDLGHIAKHKREIRILRSKRGKPVKGEESHIDLQKHIDYMGEDRFKKAARTIVNFALNTENAASKNGFYPRADVLLLENLEGLIPDAEKERGINRALAGWNRRHLVERVIEMAKDAGFKRRVFEIPPYGTSQVCSKCGALGRRYSIIRENNRREIRFGYVEKLFACPNCGYCANADHNASVNLNRRFLIEDSFKSYYDWKRLSEKKQKEEIETIESKLMDKLCAMHKISRGSISK

SEQ ID NO: 101 - protein, 769 aa, synthetic construct
MHLWRTHCVFNQRLPALLKRLFAMRRGEVGGNEAQRQVYQRVAQFVLARDAKDSVDLLNAVSLRKRSANSAFKKKATISCNGQAREVTGEEVFAEAVALASKGVFAYDKDDMRAGLPDSLFQPLTRDAVACMRSHEELVATWKKEYREWRDRKSEWEAEPEHALYLNLRPKFEEGEAARGGRFRKRAERDHAYLDWLEANPQLAAWRRKAPPAVVPIDEAGKRRIARAKAWKQASVRAEEFWKRNPELHALHKIHVQYLREFVRPRRTRRNKRREGFKQRPTFTMPDPVRHPRWCLFNAPQTSPQGYRLLRLPQSRRTVGSVELRLLTGPSDGAGFPDAWVNVRFKADPRLAQLRPVKVPRTVTRGKNKGAKVEADGFRYYDDQLLIERDAQVSGVKLLFRDIRMAPFADKPIEDRLLSATPYLVFAVEIKDEARTERAKAIRFDETSELTKSGKKRKTLPAGLVSVAVDLDTRGVGFLTRAVIGVPEIQQTHHGVRLLQSRYVAVGQVEARASGEAEWSPGPDLAHIARHKREIRRLRQLRGKPVKGERSHVRLQAHIDRMGEDRFKKAARKIVNEALRGSNPAAGDPYTRADVLLYESLETLLPDAERERGINRALLRWNRAKLIEHLKRMCDDAGIRHFPVSPFGTSQVCSKCGALGRRYSLARENGRAVIRFGWVERLFACPNPECPGRRPDRPDRPFTCNSDHNASVNLHRVFALGDQAVAAFRALAPRDSPARTLAVKRVEDTLRPQLMRVHKLADAGVDSPF

SEQ ID NO: 102 - protein, 666 aa, synthetic construct
MATLVYRYGVRAHGSARQQDAVVSDPAMLEQLRLGHELRNALVGVQHRYEDGKRAVWSGFASVAAADHRVTTGETAVAELEKQARAEHSADRTAATRQGTAESLKAARAAVKQARADRKAAMAAVAEQAKPKIQALGDDRDAEIKDLYRRFCQDGVLLPRCGRCAGDLRSDGDCTDCGAAHEPRKLYWATYNAIREDHQTAVKLVEAKRKAGQPARLRFRRWTGDGTLTVQLQRMHGPACRCVTCAEKLTRRARKTDPQAPAVAADPAYPPTDPPRDPALLASGQGKWRNVLQLGTWIPPGEWSAMSRAERRRVGRSHIGWQLGGGRQLTLPVQLHRQMPADADVAMAQLTRVRVGGRHRMSVALTAKLPDPPQVQGLPPVALHLGWRQRPDGSLRVATWACPQPLDLPPAVADVVVSHGGRWGEVIMPARWLADAEVPPRLLGRRDKAMEPVLEALADWLEAHTEACTARMTPALVRRWRSQGRLAGLTNRWRGQPPTGSAEILTYLEAWRIQDKLLWERESHLRRRLAARRDDAWRRVASWLARHAGVLVVDDADIAELRRRDDPADTDPTMPASAAQAARARAALAAPGRLRHLATITATRDGLGVHTVASAGLTRLHRKCGHQAQPDPRYAASAVVTCPGCGNGYDQDYNAAMLMLDRQQQP

SEQ ID NO: 103 - protein, 564 aa, synthetic construct
MSRVELHRAYKFRLYPTPAQVAELAEWERQLRRLYNLAHSQRLAAMQRHVRPKSPGVLKSECLSCGAVAVAEIGTDGKAKKTVKHAVGCSVLECRSCGGSPDAEGRTAHTAACSFVDYYRQGREMTQLLEEDDQLARVVCSARQETLRDLEKAWQRWHKMPGFGKPHFKKRIDSCRIYFSTPKSWAVDLGYLSFTGVASSVGRIKIRQDRVWPGDAKFSSCHVVRDVDEWYAVFPLTFTKEIEKPKGGAVGINRGAVHAIADSTGRVVDSPKFYARSLGVIRHRARLLDRKVPFGRAVKPSPTKYHGLPKADIDAAAARVNASPGRLVYEARARGSIAAAEAHLAALVLPAPRQTSQLPSEGRNRERARRFLALAHQRVRRQREWFLHNESAHYAQSYTKIAIEDWSTKEMTSSEPRDAEEMKRVTRARNRSILDVGWYELGRQIAYKSEATGAEFAKVDPGLRETETHVPEAIVRERDVDVSGMLRGEAGISGTCSRCGGLLRASASGHADAECEVCLHVEVGDVNAAVNVLKRAMFPGAAPPSKEKAKVTIGIKGRKKKRAA

SEQ ID NO: 104 - protein, 565 aa, synthetic construct
MSRVELHRAYKFRLYPTPVQVAELSEWERQLRRLYNLGHEQRLLTLTRHLRPKSPGVLKGECLSCDSTQVQEVGADGRPKTTVRHAEQCPTLACRSCGALRDAEGRTAHTVACAFVDYYRQGREMTELLAADDQLARVVCSARQEVLRDLDKAWQRWRKMPGFGKPRFKRRTDSCRIYFSTPKAWKLEGGHLSFTGAATTVGAIKMRQDRNWPASVQFSSCHVVRDVDEWYAVFPLTFVAEVARPKGGAVGINRGAVHAIADSTGRVVDSPRYYARALGVIRHRARLFDRKVPSGHAVKPSPTKYRGLSAIEVDRVARATGFTPGRVVTEALNRGGVAYAECALAAIAVLGHGPERPLTSDGRNREKARKFLALAHQRVRRQREWFLHNESAHYARTYSKIAIEDWSTKEMTASEPQGEETRRVTRSRNRSILDVGWYELGRQLAYKTEATGAEFAQVDPGLKETETNVPKAIADARDVDVSGMLRGEAGISGTCSKCGGLLRAPASGHADAECEICLNVEVGDVNAAVNVLKRAMFPGDAPPASGEKPKVSIGIKGRQKKKKAA

SEQ ID NO: 105 - protein, 499 aa, synthetic construct
MEAIATGMSPERRVELGILPGSVELKRAYKFRLYPMKVQQAELSEWERQLRRLYNLAHEQRLAALLRYRDWDFQKGACPSCRVAVPGVHTAACDHVDYFRQAREMTQLLEVDAQLSRVICCARQEVLRDLDKAWQRWRKKLGGRPRFKRRTDSCRIYLSTPKHWEIAGRYLRLSGLASSVGEIRIEQDRAFPEGALLSSCSIVRDVDEWYACLPLTFTQPIERAPHRSVGLNRGVVHALADSDGRVVDSPKFFERALATVQKRSRDLARKVSGSRNAHKARIKLAKAHQRVRRQRAAFLHQESAYYSKGFDLVALEDMSVRKMTATAGEAPEMGRGAQRDLNRGILDVGWYELARQIDYKRLAHGGELLRVDPGQTTPLACVTEEQPARGISSACAVCGIPLARPASGNARMRCTACGSSQVGDVNAAENVLTRALSSAPSGPKSPKASIKIKGRQKRLGTPANRAGEASGGDPPVRGPVEGGTLAYVVEPVSESQSDT

SEQ ID NO: 106 - protein, 560 aa, synthetic construct
MTVRTYKYRAYPTPEQAEALTSWLRFASQLYNAALEHRKNAWGRHDAHGRGFRFWDGDAAPRKKSDPPGRWVYRGGGGAHISKNDQGKLLTEFRREHAELLPPGMPALVQHEVLARLERSMAAFFQRATKGQKAGYPRWRSEHRYDSLTFGLTSPSKERFDPETGESLGRGKTVGAGTYHNGDLRLTGLGELRILEHRRIPMGAIPKSVIVRRSGKRWFVSIAMEMPSVEPAASGRPAVGLDMGVVTWGTAFTADTSAAAALVADLRRMATDPSDCRRLEELEREAAQLSEVLAHCRARGLDPARPRRCPKELTKLYRRSLHRLGELDRACARIRRRLQAAHDIAEPVPDEAGSAVLIEGSNAGMRHARRVARTQRRVARRTRAGHAHSNRRKKAVQAYARAKERERSARGDHRHKVSRALVRQFEEISVEALDIKQLTVAPEHNPDPQPDLPAHVQRRRNRGELDAAWGAFFAALDYKAADAGGRVARKPAPHTTQECARCGTLVPKPISLRVHRCPACGYTAPRTVNSARNVLQRPLEEPGRAGPSGANGRGVPHAVA

SEQ ID NO: 107 - protein, 404 aa, synthetic construct
MNCRYRYRIYPTPGQRQSLARLFGCVRVVWNDALFLCRQSEKLPKNSELQKLCITQAKKTEARGWLGQVSAIPLQQSVADLGVAFKNFFQSRSGKRKGKKVNPPRVKRRNNRQGARFTRGGFKVKTSKVYLARIGDIKIKWSRPLPSEPSSVTVIKDCAGQYFLSFVVEVKPEIKPPKNPSIGIDLGLKTFASCSNGEKIDSPDYSRLYRKLKRCQRRLAKRQRGSKRRERMRVKVAKLNAQIRDKRKDFLHKLSTKVVNENQVIALEDLNVGGMLKNRKLSRAISQAGWYEFRSLCEGKAEKHNRDFRVISRWEPTSQVCSECGYRWGKIDLSVRSIVCINCGVEHDRDDNASVNIEQAGLKVGVGHTHDSKRTGSACKTSNGAVCVEPSTHREYVQLTLFDW

SEQ ID NO: 108 - protein, 392 aa, synthetic construct
MKSRWTFRCYPTPEQEQHLARTFGCVRFVWNWALRARTDAFRAGERIGYPATDKALTLLKQQPETVWLNEVSSVCLQQALRDLQVAFSNFFDKRAAHPSFKRKEARQSANYTERGFSFDHERRILKLAKIGAIKVKWSRKAIPHPSSIRLIRTASGKYFVSLVVETQPAPMPETGESVGVDFGVARLATLSNGERISNPKHGAKWQRRLAFYQKRLARATKGSKRRMRIKRHVARIHEKIGNSRSDTLHKLSTDLVTRFDLICVEDLNLRGMVKNHSLARSLHDASIGSAIRMIEEKAERYGKNVVKIDRWFPSSKTCSDCGHIVEQLPLNVREWTCPECGTTHDRDANAAANILAVGQTVSAHGGTVRRSRAKASERKSQRSANRQGVNRA

SEQ ID NO: 109 - protein, 500 aa, synthetic construct
KEPLNIGKTAKAVFKEIDPTSLNRAANYDASIELNCKECKFKPFKNVKRYEFNFYNNWYRCNPNSCLQSTYKAQVRKVEIGYEKLKNEILTQMQYYPWFGRLYQNFFHDERDKMTSLDEIQVIGVQNKVFFNTVEKAWREIIKKRFKDNKETMETIPELKHAAGHGKRKLSNKSLLRRRFAFVQKSFKFVDNSDVSYRSFSNNIACVLPSRIGVDLGGVISRNPKREYIPQEISFNAFWKQHEGLKKGRNIEIQSVQYKGETVKRIEADTGEDKAWGKNRQRRFTSLILKLVPKQGGKKVWKYPEKRNEGNYEYFPIPIEFILDSGETSIRFGGDEGEAGKQKHLVIPFNDSKATPLASQQTLLENSRFNAEVKSCIGLAIYANYFYGYARNYVISSIYHKNSKNGQAITAIYLESIAHNYVKAIERQLQNLLLNLRDFSFMESHKKELKKYFGGDLEGTGGAQKRREKEEKIEKEIEQSYLPRLIRLSLTKMVTKQVEM

SEQ ID NO: 110 - protein, 507 aa, synthetic construct
ELIVNENKDPLNIGKTAKAVFKEIDPTSINRAANYDASIELACKECKFKPFNNTKRHDFSFYSNWHRCSPNSCLQSTYRAKIRKTEIGYEKLKNEILNQMQYYPWFGRLYQNFFNDQRDKMTSLDEIQVTGVQNKIFFNTVEKAWREIIKKRFRDNKETMRTIPDLKNKSGHGSRKLSNKSLLRRRFAFAQKSFKLVDNSDVSYRAFSNNVACVLPSKIGVDIGGIINKDLKREYIPQEITFNVFWKQHDGLKKGRNIEIHSVQYKGEIVKRIEADTGEDKAWGKNRQRRFTSLILKITPKQGGKKIWKFPEKKNASDYEYFPIPIEFILDNGDASIKFGGEEGEVGKQKHLLIPFNDSKATPLSSKQMLLETSRFNAEVKSTIGLALYANYFVSYARNYVIKSTYHKNSKKGQIVTEIYLESISQNFVRAIQRQLQSLMLNLKDWGFMQTHKKELKKYFGSDLEGSKGGQKRREKEEKIEKEIEASYLPRLIRLSLTKSVTKAEEM

SEQ ID NO: 111 - protein, 529 aa, synthetic construct
PEEKTSKLKPNSINLAANYDANEKFNCKECKFHPFKNKKRYEFNFYNNLHGCKSCTKSTNNPAVKRIEIGYQKLKFEIKNQMEAYPWFGRLRINFYSDEKRKMSELNEMQVTGVKNKIFFDAIECAWREILKKRFRESKETLITIPKLKNKAGHGARKHRNKKLLIRRRAFMKKNFHFLDNDSISYRSFANNIACVLPSKVGVDIGGIISPDVGKDIKPVDISLNLMWASKEGIKSGRKVEIYSTQYDGNMVKKIEAETGEDKSWGKNRKRRQTSLLLSIPKPSKQVQEFDFKEWPRYKDIEKKVQWRGFPIKIIFDSNHNSIEFGTYQGGKQKVLPIPFNDSKTTPLGSKMNKLEKLRFNSKIKSRLGSAIAANKFLEAARTYCVDSLYHEVSSANAIGKGKIFIEYYLEILSQNYIEAAQKQLQRFIESIEQWFVADPFQGRLKQYFKDDLKRAKCFLCANREVQTTCYAAVKLHKSCAEKVKDKNKELAIKERNNKEDAVIKEVEASNYPRVIRLKLTKTITNKAM

SEQ ID NO: 112 - protein, 726 aa, synthetic construct
SESENKIIEQYYAFLYSFRDKYEKPEFKNRGDIKRKLQNKWEDFLKEQNLKNDKKLSNYIFSNRNFRRSYDREEENEEGIDEKKSKPKRINCFEKEKNLKDQYDKDAINASANKDGAQKWGCFECIFFPMYKIESGDPNKRIIINKTRFKLFDFYLNLKGCKSCLRSTYHPYRSNVYIESNYDKLKREIGNFLQQKNIFQRMRKAKVSEGKYLTNLDEYRLSCVAMHFKNRWLFFDSIQKVLRETIKQRLKQMRESYDEQAKTKRSKGHGRAKYEDQVRMIRRRAYSAQAHKLLDNGYITLFDYDDKEINKVCLTAINQEGFDIGGYLNSDIDNVMPPIEISFHLKWKYNEPILNIESPFSKAKISDYLRKIREDLNLERGKEGKARSKKNVRRKVLASKGEDGYKKIFTDFFSKWKEELEGNAMERVLSQSSGDIQWSKKKRIHYTTLVLNINLLDKKGVGNLKYYEIAEKTKILSFDKNENKFWPITIQVLLDGYEIGTEYDEIKQLNEKTSKQFTIYDPNTKIIKIPFTDSKAVPLGMLGINIATLKTVKKTERDIKVSKIFKGGLNSKIVSKIGKGIYAGYFPTVDKEILEEVEEDTLDNEFSSKSQRNIFLKSIIKNYDKMLKEQLFDFYSFLVRNDLGVRFLTDRELQNIEDESFNLEKRFFETDRDRIARWFDNTNTDDGKEKFKKLANEIVDSYKPRLIRLPVVRVIKRIQPVKQREM

SEQ ID NO: 113 - protein, 517 aa, synthetic construct
KYSTRDFSELNEIQVTACKQDEFFKVIQNAWREIIKKRFLENRENFIEKKIFKNKKGRGKRQESDKTIQRNRASVMKNFQLIENEKIILRAPSGHVACVFPVKVGLDIGGFKTDDLEKNIFPPRTITINVFWKNRDRQRKGRKLEVWGIKARTKLIEKVHKWDKLEEVKKKRLKSLEQKQEKSLDNWSEVNNDSFYKVQIDELQEKIDKSLKGRTMNKILDNKAKESKEAEGLYIEWEKDFEGEMLRRIEASTGGEEKWGKRRQRRHTSLLLDIKNNSRGSKEIINFYSYAKQGKKEKKIEFFPFPLTITLDAEEESPLNIKSIPIEDKNATSKYFSIPFTETRATPLSILGDRVQKFKTKNISGAIKRNLGSSISSCKIVQNAETSAKSILSLPNVKEDNNMEIFINTMSKNYFRAMMKQMESFIFEMEPKTLIDPYKEKAIKWFEVAASSRAKRKLKKLSKADIKKSELLLSNTEEFEKEKQEKLEALEKEIEEFYLPRIVRLQLTKTILETPVM

SEQ ID NO: 114 - protein, 481 aa, synthetic construct
KKLQLLGHKILLKEYDPNAVNAAANFETSTAELCGQCKMKPFKNKRRFQYTFGKNYHGCLSCIQNVYYAKKRIVQIAKEELKHQLTDSIASIPYKYTSLFSNTNSIDELYILKQERAAFFSNTNSIDELYITGIENNIAFKVISAIWDEIIKKRRQRYAESLTDTGTVKANRGHGGTAYKSNTRQEKIRALQKQTLHMVTNPYISLARYKNNYIVATLPRTIGMHIGAIKDRDPQKKLSDYAINFNVFWSDDRQLIELSTVQYTGDMVRKIEAETGENNKWGENMKRTKTSLLLEILTKKTTDELTFKDWAFSTKKEIDSVTKKTYQGFPIGIIFEGNESSVKFGSQNYFPLPFDAKITPPTAEGFRLDWLRKGSFSSQMKTSYGLAIYSNKVTNAIPAYVIKNMFYKIARAENGKQIKAKFLKKYLDIAGNNYVPFIIMQHYRVLDTFEEMPISQPKVIRLSLTKTQHIIIKKDKTDSKM

SEQ ID NO: 115 - protein, 534 aa, synthetic construct
NTSNLINLGKKAINISANYDANLEVGCKNCKFLSSNGNFPRQTNVKEGCHSCEKSTYEPSIYLVKIGERKAKYDVLDSLKKFTFQSLKYQSKKSMKSRNKKPKELKEFVIFANKNKAFDVIQKSYNHLILQIKKEINRMNSKKRKKNHKRRLFRDREKQLNKLRLIESSNLFLPRENKGNNHVFTYVAIHSVGRDIGVIGSYDEKLNFETELTYQLYFNDDKRLLYAYKPKQNKIIKIKEKLWNLRKEKEPLDLEYEKPLNKSITFSIKNDNLFKVSKDLMLRRAKFNIQGKEKLSKEERKINRDLIKIKGLVNSMSYGRFDELKKEKNIWSPHIYREVRQKEIKPCLIKNGDRIEIFEQLKKKMERLRRFREKRQKKISKDLIFAERIAYNFHTKSIKNTSNKINIDQEAKRGKASYMRKRIGYETFKNKYCEQCLSKGNVYRNVQKGCSCFENPFDWIKKGDENLLPKKNEDLRVKGAFRDEALEKQIVKIAFNIAKGYEDFYDNLGESTEKDLKLKFKVGTTINEQESLKL

SEQ ID NO: 116 - protein, 537 aa, synthetic construct
TSNPIKLGKKAINISANYDSNLQIGCKNCKFLSYNGNFPRQTNVKEGCHSCEKSTYEPPVYTVRIGERRSKYDVLDSLKKFIFLSLKYRQSKKMKTRSKGIRGLEEFVISANLKKAMDVIQKSYRHLILNIKNEIVRMNGKKRNKNHKRLLFRDREKQLNKLRLIEGSSFFKPPTVKGDNSIFTCVAIHNIGRDIGIAGDYFDKLEPKIELTYQLYYEYNPKKESEINKRLLYAYKPKQNKIIEIKEKLWNLRKEKSPLDLEYEKPLTKSITFLVKRDGVFRISKDLMLRKAKFIIQGKEKLSKEERKINRDLIKIKSNIISLTYGRFDELKKDKTIWSPHIFRDVKQGKITPCIERKGDRMDIFQQLRKKSERLRENRKKRQKKISKDLIFAERIAYNFHTKSIKNTSNLINIKHEAKRGKASYMRKRIGNETFRIKYCEQCFPKNNVYKNVQKGCSCFEDPFEYIKKGNEDLIPNKNQDLKAKGAFRDDALEKQIIKVAFNIAKGYEDFYENLKKTTEKDIRLKFKVGTIISEEM

SEQ ID NO: 117 - protein, 541 aa, synthetic construct
NNSINLSKKAINISANYDANLQVRCKNCKFLSSNGNFPRQTDVKEGCHSCEKSTYEPPVYDVKIGEIKAKYEVLDSLKKFTFQSLKYQLSKSMKFRSKKIKELKEFVIFAKESKALNVINRSYKHLILNIKNDINRMNSKKRIKNHKGRLFLDRQKQLSKLKLIEGSSFFVPAKNVGNKSVFTCVAIHSIGRDIGIAGLYDSFTKPVNEITYQIFFSGERRLLYAYKPKQLKILSIKENLWSLKNEKKPLDLLYEKPLGKNLNFNVKGGDLFRVSKDLMIRNAKFNVHGRQRLSDEERLINRNFIKIKGEVVSLSYGRFEELKKDRKLWSPHIFKDVRQNKIKPCLVMQGQRIDIFEQLKRKLELLKKIRKSRQKKLSKDLIFGERIAYNFHTKSIKNTSNKINIDSDAKRGRASYMRKRIGNETFKLKYCDVCFPKANVYRRVQNGCSCSENPYNYIKKGDKDLLPKKDEGLAIKGAFRDEKLNKQIIKVAFNIAKGYEDFYDDLKKRTEKDVDLKFKIGTTVLDQKPMEIFDGIVITWL

SEQ ID NO: 118 - protein, 542 aa, synthetic construct
LLTTVVETNNLAKKAINVAANFDANIDRQYYRCTPNLCRFIAQSPRETKEKDAGCSSCTQSTYDPKVYVIKIGKLLAKYEILKSLKRFLFMNRYFKQKKTERAQQKQKIGTELNEMSIFAKATNAMEVIKRATKHCTYDIIPETKSLQMLKRRRHRVKVRSLLKILKERRMKIKKIPNTFIEIPKQAKKNKSDYYVAAALKSCGIDVGLCGAYEKNAEVEAEYTYQLYYEYKGNSSTKRILYCYNNPQKNIREFWEAFYIQGSKSHVNTPGTIRLKMEKFLSPITIESEALDFRVWNSDLKIRNGQYGFIKKRSLGKEAREIKKGMGDIKRKIGNLTYGKSPSELKSIHVYRTERENPKKPRAARKKEDNFMEIFEMQRKKDYEVNKKRRKEATDAAKIMDFAEEPIRHYHTNNLKAVRRIDMNEQVERKKTSVFLKRIMQNGYRGNYCRKCIKAPEGSNRDENVLEKNEGCLDCIGSEFIWKKSSKEKKGLWHTNRLLRRIRLQCFTTAKAYENFYNDLFEKKESSLDIIKLKVSITTKSM

SEQ ID NO: 119 - protein, 564 aa, synthetic construct
ASTMNLAKQAINFAANYDSNLEIGCKGCKFMSTWSKKSNPKFYPRQNNQANKCHSCTYSTGEPEVPIIEIGERAAKYKIFTALKKFVFMSVAYKERRRQRFKSKKPKELKELAICSNREKAMEVIQKSVVHCYGDVKQEIPRIRKIKVLKNHKGRLFYKQKRSKIKIAKLEKGSFFKTFIPKVHNNGCHSCHEASLNKPILVTTALNTIGADIGLINDYSTIAPTETDISWQVYYEFIPNGDSEAVKKRLLYFYKPKGALIKSIRDKYFKKGHENAVNTGFFKYQGKIVKGPIKFVNNELDFARKPDLKSMKIKRAGFAIPSAKRLSKEDREINRESIKIKNKIYSLSYGRKKTLSDKDIIKHLYRPVRQKGVKPLEYRKAPDGFLEFFYSLKRKERRLRKQKEKRQKDMSEIIDAADEFAWHRHTGSIKKTTNHINFKSEVKRGKVPIMKKRIANDSFNTRHCGKCVKQGNAINKYYIEKQKNCFDCNSIEFKWEKAALEKKGAFKLNKRLQYIVKACFNVAKAYESFYEDFRKGEEESLDLKFKIGTTTTLKQYPQNKARAM

SEQ ID NO: 120 - protein, 610 aa, synthetic construct
HSHNLMLTKLGKQAINFAANYDANLEIGCKNCKFLSYSPKQANPKKYPRQTDVHEDGNIACHSCMQSTKEPPVYIVPIGERKSKYEILTSLNKFTFLALKYKEKKRQAFRAKKPKELQELAIAFNKEKAIKVIDKSIQHLILNIKPEIARIQRQKRLKNRKGKLLYLHKRYAIKMGLIKNGKYFKVGSPKKDGKKLLVLCALNTIGRDIGIIGNIEENNRSETEITYQLYFDCLDANPNELRIKEIEYNRLKSYERKIKRLVYAYKPKQTKILEIRSKFFSKGHENKVNTGSFNFENPLNKSISIKVKNSAFDFKIGAPFIMLRNGKFHIPTKKRLSKEEREINRTLSKIKGRVFRLTYGRNISEQGSKSLHIYRKERQHPKLSLEIRKQPDSFIDEFEKLRLKQNFISKLKKQRQKKLADLLQFADRIAYNYHTSSLEKTSNFINYKPEVKRGRTSYIKKRIGNEGFEKLYCETCIKSNDKENAYAVEKEELCFVCKAKPFTWKKTNKDKLGIFKYPSRIKDFIRAAFTVAKSYNDFYENLKKKDLKNEIFLKFKIGLILSHEKKNHISIAKSVAEDERISGKSIKNILNKSIKLEKNCYSCFFHKEDM

SEQ ID NO: 121 - protein, 552 aa, synthetic construct
SLERVIDKRNLAKKAINIAANFDANINKGFYRCETNQCMFIAQKPRKTNNTGCSSCLQSTYDPVIYVVKVGEMLAKYEILKSLKRFVFMNRSFKQKKTEKAKQKERIGGELNEMSIFANAALAMGVIKRAIRHCHVDIRPEINRLSELKKTKHRVAAKSLVKIVKQRKTKWKGIPNSFIQIPQKARNKDADFYVASALKSGGIDIGLCGTYDKKPHADPRWTYQLYFDTEDESEKRLLYCYNDPQAKIRDFWKTFYERGNPSMVNSPGTIEFRMEGFFEKMTPISIESKDFDFRVWNKDLLIRRGLYEIKKRKNLNRKAREIKKAMGSVKRVLANMTYGKSPTDKKSIPVYRVEREKPKKPRAVRKEENELADKLENYRREDFLIRNRRKREATEIAKIIDAAEPPIRHYHTNHLRAVKRIDLSKPVARKNTSVFLKRIMQNGYRGNYCKKCIKGNIDPNKDECRLEDIKKCICCEGTQNIWAKKEKLYTGRINVLNKRIKQMKLECFNVAKAYENFYDNLAALKEGDLKVLKLKVSIPALNPEASDPEEDM

SEQ ID NO: 122 - protein, 534 aa, synthetic construct
NASINLGKRAINLSANYDSNLVIGCKNCKFLSFNGNFPRQTNVREGCHSCDKSTYAPEVYIVKIGERKAKYDVLDSLKKFTFQSLKYQIKKSMRERSKKPKELLEFVIFANKDKAFNVIQKSYEHLILNIKQEINRMNGKKRIKNHKKRLFKDREKQLNKLRLIGSSSLFFPRENKGDKDLFTYVAIHSVGRDIGVAGSYESHIEPISDLTYQLFINNEKRLLYAYKPKQNKIIELKENLWNLKKEKKPLDLEFTKPLEKSITFSVKNDKLFKVSKDLMLRQAKFNIQGKEKLSKEERQINRDFSKIKSNVISLSYGRFEELKKEKNIWSPHIYREVKQKEIKPCIVRKGDRIELFEQLKRKMDKLKKFRKERQKKISKDLNFAERIAYNFHTKSIKNTSNKINIDQEAKRGKASYMRKRIGNESFRKKYCEQCFSVGNVYHNVQNGCSCFDNPIELIKKGDEGLIPKGKEDRKYKGALRDDNLQMQIIRVAFNIAKGYEDFYNNLKEKTEKDLKLKFKIGTTISTQESNNKEM

SEQ ID NO: 123 - protein, 577 aa, synthetic construct
SNLIKLGKQAINFAANYDANLEVGCKNCKFLSSTNKYPRQTNVHLDNKMACRSCNQSTMEPAIYIVRIGEKKAKYDIYNSLTKFNFQSLKYKAKRSQRFKPKQPKELQELSIAVRKEKALDIIQKSIDHLIQDIRPEIPRIKQQKRYKNHVGKLFYLQKRRKNKLNLIGKGSFFKVFSPKEKKNELLVICALTNIGRDIGLIGNYNTIINPLFEVTYQLYYDYIPKKNNKNVQRRLLYAYKSKNEKILKLKEAFFKRGHENAVNLGSFSYEKPLEKSLTLKIKNDKDDFQVSPSLRIRTGRFFVPSKRNLSRQEREINRRLVKIKSKIKNMTYGKFETARDKQSVHIFRLERQKEKLPLQFRKDEKEFMEEFQKLKRRTNSLKKLRKSRQKKLADLLQLSEKVVYNNHTGTLKKTSNFLNFSSSVKRGKTAYIKELLGQEGFETLYCSNCINKGQKTRYNIETKEKCFSCKDVPFVWKKKSTDKDRKGAFLFPAKLKDVIKATFTVAKAYEDFYDNLKSIDEKKPYIKFKIGLILAHVRHEHKARAKEEAGQKNIYNKPIKIDKNCKECFFFKEEAM

SEQ ID NO: 124 - protein, 613 aa, synthetic construct
NTTRKKFRKRTGFPQSDNIKLAYCSAIVRAANLDADIQKKHNQCNPNLCVGIKSNEQSRKYEHSDRQALLCYACNQSTGAPKVDYIQIGEIGAKYKILQMVNAYDFLSLAYNLTKLRNGKSRGHQRMSQLDEVVIVADYEKATEVIKRSINHLLDDIRGQLSKLKKRTQNEHITEHKQSKIRRKLRKLSRLLKRRRWKWGTIPNPYLKNWVFTKKDPELVTVALLHKLGRDIGLVNRSKRRSKQKLLPKVGFQLYYKWESPSLNNIKKSKAKKLPKRLLIPYKNVKLFDNKQKLENAIKSLLESYQKTIKVEFDQFFQNRTEEIIAEEQQTLERGLLKQLEKKKNEFASQKKALKEEKKKIKEPRKAKLLMEESRSLGFLMANVSYALFNTTIEDLYKKSNVVSGCIPQEPVVVFPADIQNKGSLAKILFAPKDGFRIKFSGQHLTIRTAKFKIRGKEIKILTKTKREILKNIEKLRRVWYREQHYKLKLFGKEVSAKPRFLDKRKTSIERRDPNKLADQTDDRQAELRNKEYELRHKQHKMAERLDNIDTNAQNLQTLSFWVGEADKPPKLDEKDARGFGVRTCISAWKWFMEDLLKKQEEDPLLKLKLSIM

SEQ ID NO: 125 - protein, 615 aa, synthetic construct
PKKPKFQKRTGFPQPDNLRKEYCLAIVRAANLDADFEKKCTKCEGIKTNKKGNIVKGRTYNSADKDNLLCYACNISTGAPAVDYVFVGALEAKYKILQMVKAYDFHSLAYNLAKLWKGRGRGHQRMGGLNEVVIVSNNEKALDVIEKSLNHFHDEIRGELSRLKAKFQNEHLHVHKESKLRRKLRKISRLLKRRRWKWDVIPNSYLRNFTFTKTRPDFISVALLHRVGRDIGLVTKTKIPKPTDLLPQFGFQIYYTWDEPKLNKLKKSRLRSEPKRLLVPYKKIELYKNKSVLEEAIRHLAEVYTEDLTICFKDFFETQKRKFVSKEKESLKRELLKELTKLKKDFSERKTALKRDRKEIKEPKKAKLLMEESRSLGFLAANTSYALFNLIAADLYTKSKKACSTKLPRQLSTILPLEIKEHKSTTSLAIKPEEGFKIRFSNTHLSIRTPKFKMKGADIKALTKRKREILKNATKLEKSWYGLKHYKLKLYGKEVAAKPRFLDKRNPSIDRRDPKELMEQIENRRNEVKDLEYEIRKGQHQMAKRLDNVDTNAQNLQTKSFWVGEADKPPELDSMEAKKLGLRTCISAWKWFMKDLVLLQEKSPNLKLKLSLTEM

SEQ ID NO: 126 - protein, 775 aa, synthetic construct
KFSKRQEGFLIPDNIDLYKCLAIVRSANLDADVQGHKSCYGVKKNGTYRVKQNGKKGVKEKGRKYVFDLIAFKGNIEKIPHEAIEEKDQGRVIVLGKFNYKLILNIEKNHNDRASLEIKNKIKKLVQISSLETGEFLSDLLSGKIGIDEVYGIIEPDVFSGKELVCKACQQSTYAPLVEYMPVGELDAKYKILSAIKGYDFLSLAYNLSRNRANKKRGHQKLGGGELSEVVISANYDKALNVIKRSINHYHVEIKPEISKLKKKMQNEPLKVMKQARIRRELHQLSRKVKRLKWKWGMIPNPELQNIIFEKKEKDFVSYALLHTLGRDIGLFKDTSMLQVPNISDYGFQIYYSWEDPKLNSIKKIKDLPKRLLIPYKRLDFYIDTILVAKVIKNLIELYRKSYVYETFGEEYGYAKKAEDILFDWDSINLSEGIEQKIQKIKDEFSDLLYEARESKRQNFVESFENILGLYDKNFASDRNSYQEKIQSMIIKKQQENIEQKLKREFKEVIERGFEGMDQNKKYYKVLSPNIKGGLLYTDTNNLGFFRSHLAFMLLSKISDDLYRKNNLVSKGGNKGILDQTPETMLTLEFGKSNLPNISIKRKFFNIKYNSSWIGIRKPKFSIKGAVIREITKKVRDEQRLIKSLEGVWHKSTHFKRWGKPRFNLPRHPDREKNNDDNLMESITSRREQIQLLLREKQKQQEKMAGRLDKIDKEIQNLQTANFQIKQIDKKPALTEKSEGKQSVRNALSAWKWFMEDLIKYQKRTPILQLKLAKM

SEQ ID NO: 127 - protein, 777 aa, synthetic construct
KFSKRQEGFVIPENIGLYKCLAIVRSANLDADVQGHVSCYGVKKNGTYVLKQNGKKSIREKGRKYASDLVAFKGDIEKIPFEVIEEKKKEQSIVLGKFNYKLVLDVMKGEKDRASLTMKNKSKKLVQVSSLGTDEFLLTLLNEKFGIEEIYGIIEPEVFSGKKLVCKACQQSTYAPLVEYMPVGELDSKYKILSAIKGYDFLSLAYNLARHRSNKKRGHQKLGGGELSEVVISANNAKALNVIKRSLNHYYSEIKPEISKLRKKMQNEPLKVGKQARMRRELHQLSRKVKRLKWKWGKIPNLELQNITFKESDRDFISYALLHTLGRDIGMFNKTEIKMPSNILGYGFQIYYDWEEPKLNTIKKSKNTPKRILIPYKKLDFYNDSILVARAIKELVGLFQESYEWEIFGNEYNYAKEAEVELIKLDEESINGNVEKKLQRIKENFSNLLEKAREKKRQNFIESFESIARLYDESFTADRNEYQREIQSFIIEKQKQSIEKKLKNEFKKIVEKKFNEQEQGKKHYRVLNPTIINEFLPKDKNNLGFLRSKIAFILLSKISDDLYKKSNAVSKGGEKGIIKQQPETILDLEFSKSKLPSINIKKKLFNIKYTSSWLGIRKPKFNIKGAKIREITRRVRDVQRTLKSAESSWYASTHFRRWGFPRFNQPRHPDKEKKSDDRLIESITLLREQIQILLREKQKGQKEMAGRLDDVDKKIQNLQTANFQIKQTGDKPALTEKSAGKQSFRNALSAWKWFMENLLKYQNKTPDLKLKIARTVM

SEQ ID NO: 128 - protein, 610 aa, synthetic construct
KWIEPNNIDFNKCLAITRSANLDADVQGHKMCYGIKTNGTYKAIGKINKKHNTGIIEKRRTYVYDLIVTKEKNEKIVKKTDFMAIDEEIEFDEKKEKLLKKYIKAEVLGTGELIRKDLNDGEKFDDLCSIEEPQAFRRSELVCKACNQSTYASDIRYIPIGEIEAKYKILKAIKGYDFLSLKYNLGRLRDSKKRGHQKMGQGELKEFVICANKEKALDVIKRSLNHYLNEVKDEISRLNKKMQNEPLKVNDQARWRRELNQISRRLKRLKWKWGEIPNPELKNLIFKSSRPEFVSYALIHTLGRDIGLINETELKPNNIQEYGFQIYYKWEDPELNHIKKVKNIPKRFIIPYKNLDLFGKYTILSRAIEGILKLYSSSFQYKSFKDPNLFAKEGEKKITNEDFELGYDEKIKKIKDDFKSYKKALLEKKKNTLEDSLNSILSVYEQSLLTEQINNVKKWKEGLLKSKESIHKQKKIENIEDIISRIEELKNVEGWIRTKERDIVNKEETNLKREIKKELKDSYYEEVRKDFSDLKKGEESEKKPFREEPKPIVIKDYIKFDVLPGENSALGFFLSHLSFNLFDSIQYELFEKSRLSSSKHPQIPETILDL

SEQ ID NO: 129 - protein, 632 aa, synthetic construct
FRKFVKRSGAPQPDNLNKYKCIAIVRAANLDADIMSNESSNCVMCKGIKMNKRKTAKGAAKTTELGRVYAGQSGNLLCTACTKSTMGPLVDYVPIGRIRAKYTILRAVKEYDFLSLAYNLARTRVSKKGGRQKMHSLSELVIAAEYEIAWNIIKSSVIHYHQETKEEISGLRKKLQAEHIHKNKEARIRREMHQISRRIKRLKWKWHMIPNSELHNFLFKQQDPSFVAVALLHTLGRDIGMINKPKGSAKREFIPEYGFQIYYKWMNPKLNDINKQKYRKMPKRSLIPYKNLNVFGDRELIENAMHKLLKLYDENLEVKGSKFFKTRVVAISSKESEKLKRDLLWKGELAKIKKDFNADKNKMQELFKEVKEPKKANALMKQSRNMGFLLQNISYGALGLLANRMYEASAKQSKGDATKQPSIVIPLEMEFGNAFPKLLLRSGKFAMNVSSPWLTIRKPKFVIKGNKIKNITKLMKDEKAKLKRLETSYHRATHFRPTLRGSIDWDSPYFSSPKQPNTHRRSPDRLSADITEYRGRLKSVEAELREGQRAMAKKLDSVDMTASNLQTSNFQLEKGEDPRLTEIDEKGRSIRNCISSWKKFMEDLMKAQEANPVIKIKIALKDESSVLSEDSM

SEQ ID NO: 130 - protein, 625 aa, synthetic construct
KFHPENLNKSYCLAIVRAANLDADIQGHINCIGIKSNKSDRNYENKLESLQNVELLCKACTKSTYKPNINSVPVGEKKAKYSILSEIKKYDFNSLVYNLKKYRKGKSRGHQKLNELRELVITSEYKKALDVINKSVNHYLVNIKNKMSKLKKILQNEHIHVGTLARIRRERNRISRKLDHYRKKWKFVPNKILKNYVFKNQSPDFVSVALLHKLGRDIGLITKTAILQKSFPEYSLQLYYKYDTPKLNYLKKSKFKSLPKRILISYKYPKFDINSNYIEESIDKLLKLYEESPIYKNNSKIIEFFKKSEDNLIKSENDSLKRGIMKEFEKVTKNFSSKKKKLKEELKLKNEDKNSKMLAKVSRPIGFLKAYLSYMLFNIISNRIFEFSRKSSGRIPQLPSCIINLGNQFENFKNELQDSNIGSKKNYKYFCNLLLKSSGFNISYEEEHLSIKTPNFFINGRKLKEITSEKKKIRKENEQLIKQWKKLTFFKPSNLNGKKTSDKIRFKSPNNPDIERKSEDNIVENIAKVKYKLEDLLSEQRKEFNKLAKKHDGVDVEAQCLQTKSFWIDSNSPIKKSLEKKNEKVSVKKKMKAIRSCISAWKWFMADLIEAQKETPMIKLKLALM

SEQ ID NO: 131 - protein, 517 aa, synthetic construct
TTLVPSHLAGIEVMDETTSRNEDMIQKETSRSNEDENYLGVKNKCGINVHKSGRGSSKHEPNMPPEKSGEGQMPKQDSTEMQQRFDESVTGETQVSAGATASIKTDARANSGPRVGTARALIVKASNLDRDIKLGCKPCEYIRSELPMGKKNGCNHCEKSSDIASVPKVESGFRKAKYELVRRFESFAADSISRHLGKEQARTRGKRGKKDKKEQMGKVNLDEIAILKNESLIEYTENQILDARSNRIKEWLRSLRLRLRTRNKGLKKSKSIRRQLITLRRDYRKWIKPNPYRPDEDPNENSLRLHTKLGVDIGVQGGDNKRMNSDDYETSFSITWRDTATRKICFTKPKGLLPRHMKFKLRGYPELILYNEELRIQDSQKFPLVDWERIPIFKLRGVSLGKKKVKALNRITEAPRLVVAKRIQVNIESKKKKVLTRYVYNDKSINGRLVKAEDSNKDPLLEFKKQAEEINSDAKYYENQEIAKNYLWGCEGLHKNLLEEQTKNPYLAFKYGFLNIV

SEQ ID NO: 132 - protein, 410 aa, synthetic construct
LDFKRTCSQELVLLPEIEGLKLSGTQGVTSLAKKLINKAANVDRDESYGCHHCIHTRTSLSKPVKKDCNSCNQSTNHPAVPITLKGYKIAFYELWHRFTSWAVDSISKALHRNKVMGKVNLDEYAVVDNSHIVCYAVRKCYEKRQRSVRLHKRAYRCRAKHYNKSQPKVGRIYKKSKRRNARNLKKEAKRYFQPNEITNGSSDALFYKIGVDLGIAKGTPETEVKVDVSICFQVYYGDARRVLRVRKMDELQSFHLDYTGKLKLKGIGNKDTFTIAKRNESLKWGSTKYEVSRAHKKFKPFGKKGSVKRKCNDYFRSIASWSCEAASQRAQSNLKNAFPYQKALVKCYKNLDYKGVKKNDMWYRLCSNRIFRYSRIAEDIAQYQSDKGKAKFEFVILAQSVAEYDISAIM

SEQ ID NO: 133 - protein, 602 aa, synthetic construct
VFLTDDKRKTALRKIRSAFRKTAEIALVRAQEADSLDRQAKKLTIETVSFGAPGAKNAFIGSLQGYNWNSHRANVPSSGSAKDVFRITELGLGIPQSAHEASIGKSFELVGNVVRYTANLLSKGYKKGAVNKGAKQQREIKGKEQLSFDLISNGPISGDKLINGQKDALAWWLIDKMGFHIGLAMEPLSSPNTYGITLQAFWKRHTAPRRYSRGVIRQWQLPFGRQLAPLIHNFFRKKGASIPIVLTNASKKLAGKGVLLEQTALVDPKKWWQVKEQVTGPLSNIWERSVPLVLYTATFTHKHGAAHKRPLTLKVIRISSGSVFLLPLSKVTPGKLVRAWMPDINILRDGRPDEAAYKGPDLIRARERSFPLAYTCVTQIADEWQKRALESNRDSITPLEAKLVTGSDLLQIHSTVQQAVEQGIGGRISSPIQELLAKDALQLVLQQLFMTVDLLRIQWQLKQEVADGNTSEKAVGWAIRISNIHKDAYKTAIEPCTSALKQAWNPLSGFEERTFQLDASIVRKRSTAKTPDDELVIVLRQQAAEMTVAVTQSVSKELMELAVRHSATLHLLVGEVASKQLSRSADKDRGAMDHWKLLSQSM

SEQ ID NO: 134 - protein, 494 aa, synthetic construct
EDLLQKALNTATNVAAIERHSCISCLFTESEIDVKYKTPDKIGQNTAGCQSCTFRVGYSGNSHTLPMGNRIALDKLRETIQRYAWHSLLFNVPPAPTSKRVRAISELRVAAGRERLFTVITFVQTNILSKLQKRYAANWTPKSQERLSRLREEGQHILSLLESGSWQQKEVVREDQDLIVCSALTKPGLSIGAFCRPKYLKPAKHALVLRLIFVEQWPGQIWGQSKRTRRMRRRKDVERVYDISVQAWALKGKETRISECIDTMRRHQQAYIGVLPFLILSGSTVRGKGDCPILKEITRMRYCPNNEGLIPLGIFYRGSANKLLRVVKGSSFTLPMWQNIETLPHPEPFSPEGWTATGALYEKNLAYWSALNEAVDWYTGQILSSGLQYPNQNEFLARLQNVIDSIPRKWFRPQGLKNLKPNGQEDIVPNEFVIPQNAIRAHHVIEWYHKTNDLVAKTLLGWGSQTTLNQTRPQGDLRFTYTRYYFREKEVPEV

SEQ ID NO: 135 - protein, 649 aa, synthetic construct
VPKKKLMRELAKKAVFEAIFNDPIPGSFGCKRCTLIDGARVTDAIEKKQGAKRCAGCEPCTFHTLYDSVKHALPAATGCDRTAIDTGLWEILTALRSYNWMSFRRNAVSDASQKQVWSIEELAIWADKERALRVILSALTHTIGKLKNGFSRDGVWKGGKQLYENLAQKDLAKGLFANGEIFGKELVEADHDMLAWTIVPNHQFHIGLIRGNWKPAAVEASTAFDARWLTNGAPLRDTRTHGHRGRRFNRTEKLTVLCIKRDGGVSEEFRQERDYELSVMLLQPKNKLKPEPKGELNSFEDLHDHWWFLKGDEATALVGLTSDPTVGDFIQLGLYIRNPIKAHGETKRRLLICFEPPIKLPLRRAFPSEAFKTWEPTINVFRNGRRDTEAYYDIDRARVFEFPETRVSLEHLSKQWEVLRLEPDRENTDPYEAQQNEGAELQVYSLLQEAAQKMAPKVVIDPFGQFPLELFSTFVAQLFNAPLSDTKAKIGKPLDSGFVVESHLHLLEEDFAYRDFVRVTFMGTEPTFRVIHYSNGEGYWKKTVLKGKNNIRTALIPEGAKAAVDAYKNKRCPLTLEAAILNEEKDRRLVLGNKALSLLAQTARGNLTILEALAAEVLRPLSGTEGVVHLHACVTRHSTLTESTETDNM

SEQ ID NO: 136 - protein, 414 aa, synthetic construct
VEKLFSERLKRAMWLKNEAGRAPPAETLTLKHKRVSGGHEKVKEELQRVLRSLSGTNQAAWNLGLSGGREPKSSDALKGEKSRVVLETVVFHSGHNRVLYDVIEREDQVHQRSSIMHMRRKGSNLLRLWGRSGKVRRKMREEVAEIKPVWHKDSRWLAIVEEGRQSVVGISSAGLAVFAVQESQCTTAEPKPLEYVVSIWFRGSKALNPQDRYLEFKKLKTTEALRGQQYDPIPFSLKRGAGCSLAIRGEGIKFGSRGPIKQFFGSDRSRPSHADYDGKRRLSLFSKYAGDLADLTEEQWNRTVSAFAEDEVRRATLANIQDFLSISHEKYAERLKKRIESIEEPVSASKLEAYLSAIFETFVQQREALASNFLMRLVESVALLISLEEKSPRVEFRVARYLAESKEGFNRKAM

SEQ ID NO: 137 - protein, 413 aa, synthetic construct
VVITQSELYKERLLRVMEIKNDRGRKEPRESQGLVLRFTQVTGGQEKVKQKLWLIFEGFSGTNQASWNFGQPAGGRKPNSGDALKGPKSRVTYETVVFHFGLRLLSAVIERHNLKQQRQTMAYMKRRAAARKKWARSGKKCSRMRNEVEKIKPKWHKDPRWFDIVKEGEPSIVGISSAGFAIYIVEEPNFPRQDPLEIEYAISIWFRRDRSQYLTFKKIQKAEKLKELQYNPIPFRLKQEKTSLVFESGDIKFGSRGSIEHFRDEARGKPPKADMDNNRRLTMFSVFSGNLTNLTEEQYARPVSGLLAPDEKRMPTLLKKLQDFFTPIHEKYGERIKQRLANSEASKRPFKKLEEYLPAIYLEFRARREGLASNWVLVLINSVRTLVRIKSEDPYIEFKVSQYLLEKEDNKAL

SEQ ID NO: 138 - protein, 449 aa, synthetic construct
KQDALFEERLKKAIFIKRQADPLQREELSLLPPNRKIVTGGHESAKDTLKQILRAINGTNQASWNPGTPSGKRDSKSADALAGPKSRVKLETVVFHVGHRLLKKVVEYQGHQKQQHGLKAFMRTCAAMRKKWKRSGKVVGELREQLANIQPKWHYDSRPLNLCFEGKPSVVGLRSAGIALYTIQKSVVPVKEPKPIEYAVSIWFRGPKAMDREDRCLEFKKLKIATELRKLQFEPIVSTLTQGIKGFSLYIQGNSVKFGSRGPIKYFSNESVRQRPPKADPDGNKRLALFSKFSGDLSDLTEEQWNRPILAFEGIIRRATLGNIQDYLTVGHEQFAISLEQLLSEKESVLQMSIEQQRLKKNLGKKAENEWVESFGAEQARKKAQGIREYISGFFQEYCSQREQWAENWVQQLNKSVRLFLTIQDSTPFIEFRVARYLPKGEKKKGKAM

SEQ ID NO: 139 - protein, 711 aa, synthetic construct
ANHAERHKRLRKEANRAANRNRPLVADCDTGDPLVGICRLLRRGDKMQPNKTGCRSCEQVEPELRDAILVSGPGRLDNYKYELFQRGRAMAVHRLLKRVPKLNRPKKAAGNDEKKAENKKSEIQKEKQKQRRMMPAVSMKQVSVADFKHVIENTVRHLFGDRRDREIAECAALRAASKYFLKSRRVRPRKLPKLANPDHGKELKGLRLREKRAKLKKEKEKQAELARSNQKGAVLHVATLKKDAPPMPYEKTQGRNDYTTFVISAAIKVGATRGTKPLLTPQPREWQCSLYWRDGQRWIRGGLLGLQAGIVLGPKLNRELLEAVLQRPIECRMSGCGNPLQVRGAAVDFFMTTNPFYVSGAAYAQKKFKPFGTKRASEDGAAAKAREKLMTQLAKVLDKVVTQAAHSPLDGIWETRPEAKLRAMIMALEHEWIFLRPGPCHNAAEEVIKCDCTGGHAILWALIDEARGALEHKEFYAVTRAHTHDCEKQKLGGRLAGFLDLLIAQDVPLDDAPAARKIKTLLEATPPAPCYKAATSIATCDCEGKFDKLWAIIDATRAGHGTEDLWARTLAYPQNVNCKCKAGKDLTHRLADFLGLLIKRDGPFRERPPHKVTGDRKLVFSGDKKCKGHQYVILAKAHNEEVVRAWISRWGLKSRTNKAGYAATELNLLLNWLSICRRRWMDMLTVQRDTPYIRMKTGRLVVDDKKERKAM

SEQ ID NO: 140 - protein, 574 aa, synthetic construct
AKQREALRVALERGIVRASNRTYTLVTNCTKGGPLPEQCRMIERGKARAMKWEPKLVGCGSCAAATVDLPAIEEYAQPGRLDVAKYKLTTQILAMATRRMMVRAAKLSRRKGQWPAKVQEEKEEPPEPKKMLKAVEMRPVAIVDFNRVIQTTIEHLWAERANADEAELKALKAAAAYFGPSLKIRARGPPKAAIGRELKKAHRKKAYAERKKARRKRAELARSQARGAAAHAAIRERDIPPMAYERTQGRNDVTTIPIAAAIKIAATRGARPLPAPKPMKWQCSLYWNEGQRWIRGGMLTAQAYAHAANIHRPMRCEMWGVGNPLKVRAFEGRVADPDGAKGRKAEFRLQTNAFYVSGAAYRNKKFKPFGTDRGGIGSARKKRERLMAQLAKILDKVVSQAAHSPLDDIWHTRPAQKLRAMIKQLEHEWMFLRPQAPTVEGTKPDVDVAGNMQRQIKALMAPDLPPIEKGSPAKRFTGDKRKKGERAVRVAEAHSDEVVTAWISRWGIQTRRNEGSYAAQELELLLNWLQICRRRWLDMTAAQRVSPYIRMKSGRMITDAADEGVAPIPLVENM

SEQ ID NO: 141 - protein, 743 aa, synthetic construct
KSISGRSIKHMACLKDMLKSEITEIEEKQKKESLRKWDYYSKFSDEILFRRNLNVSANHDANACYGCNPCAFLKEVYGFRIERRNNERIISYRRGLAGCKSCVQSTGYPPIEFVRRKFGADKAMEIVREVLHRRNWGALARNIGREKEADPILGELNELLLVDARPYFGNKSAANETNLAFNVITRAAKKFRDEGMYDIHKQLDIHSEEGKVPKGRKSRLIRIERKHKAIHGLDPGETWRYPHCGKGEKYGVWLNRSRLIHIKGNEYRCLTAFGTTGRRMSLDVACSVLGHPLVKKKRKKGKKTVDGTELWQIKKATETLPEDPIDCTFYLYAAKPTKDPFILKVGSLKAPRWKKLHKDFFEYSDTEKTQGQEKGKRVVRRGKVPRILSLRPDAKFKVSIWDDPYNGKNKEGTLLRMELSGLDGAKKPLILKRYGEPNTKPKNFVFWRPHITPHPLTFTPKHDFGDPNKKTKRRRVFNREYYGHLNDLAKMEPNAKFFEDREVSNKKNPKAKNIRIQAKESLPNIVAKNGRWAAFDPNDSLWKLYLHWRGRRKTIKGGISQEFQEFKERLDLYKKHEDESEWKEKEKLWENHEKEWKKTLEIHGSIAEVSQRCVMQSMMGPLDGLVQKKDYVHIGQSSLKAADDAWTFSANRYKKATGPKWGKISVSNLLYDANQANAELISQSISKYLSKQKDNQGCEGRKMKFLIKIIEPLRENFVKHTRWLHEMTQKDCEVRAQFSRVSM

SEQ ID NO: 142 - protein, 769 aa, synthetic construct
FPSDVGADALKHVRMLQPRLTDEVRKVALTRAPSDRPALARFAAVAQDGLAFVRHLNVSANHDSNCTFPRDPRDPRRGPCEPNPCAFLREVWGFRIVARGNERALSYRRGLAGCKSCVQSTGFPSVPFHRIGADDCMRKLHEILKARNWRLLARNIGREREADPLLTELSEYLLVDARTYPDGAAPNSGRLAENVIKRAAKKFRDEGMRDIHAQLRVHSREGKVPKGRLQRLRRIERKHRAIHALDPGPSWEAEGSARAEVQGVAVYRSQLLRVGHHTQQIEPVGIVARTLFGVGRTDLDVAVSVLGAPLTKRKKGSKTLESTEDFRIAKARETRAEDKIEVAFVLYPTASLLRDEIPKDAFPAMRIDRFLLKVGSVQADREILLQDDYYRFGDAEVKAGKNKGRTVTRPVKVPRLQALRPDAKFRVNVWADPFGAGDSPGTLLRLEVSGVTRRSQPLRLLRYGQPSTQPANFLCWRPHRVPDPMTFTPRQKFGERRKNRRTRRPRVFERLYQVHIKHLAHLEPNRKWFEEARVSAQKWAKARAIRRKGAEDIPVVAPPAKRRWAALQPNAELWDLYAHDREARKRFRGGRAAEGEEFKPRLNLYLAHEPEAEWESKRDRWERYEKKWTAVLEEHSRMCAVADRTLPQFLSDPLGARMDDKDYAFVGKSALAVAEAFVEEGTVERAQGNCSITAKKKFASNASRKRLSVANLLDVSDKADRALVFQAVRQYVQRQAENGGVEGRRMAFLRKLLAPLRQNFVCHTRWLHM

SEQ ID NO: 143 - protein, 564 aa, synthetic construct
AARKKKRGKIGITVKAKEKSPPAAGPFMARKLVNVAANVDGVEVHLCVECEADAHGSASARLLGGCRSCTGSIGAEGRLMGSVDVDRERVIAEPVHTETERLGPDVKAFEAGTAESKYAIQRGLEYWGVDLISRNRARTVRKMEEADRPESSTMEKTSWDEIAIKTYSQAYHASENHLFWERQRRVRQHALALFRRARERNRGESPLQSTQRPAPLVLAALHAEAAAISGRARAEYVLRGPSANVRAAAADIDAKPLGHYKTPSPKVARGFPVKRDLLRARHRIVGLSRAYFKPSDVVRGTSDAIAHVAGRNIGVAGGKPKEIEKTFTLPFVAYWEDVDRVVHCSSFKADGPWVRDQRIKIRGVSSAVGTFSLYGLDVAWSKPTSFYIRCSDIRKKFHPKGFGPMKHWRQWAKELDRLTEQRASCVVRALQDDEELLQTMERGQRYYDVFSCAATHATRGEADPSGGCSRCELVSCGVAHKVTKKAKGDTGIEAVAVAGCSLCESKLVGPSKPRVHRQMAALRQSHALNYLRRLQREWEALEAVQAPTPYLRFKYARHLEVRSM

SEQ ID NO: 144 - protein, 565 aa, synthetic construct
AAKKKKQRGKIGISVKPKEGSAPPADGPFMARKLVNVAANVDGVEVNLCIECEADAHGSAPARLLGGCKSCTGSIGAEGRLMGSVDVDRADAIAKPVNTETEKLGPDVQAFEAGTAETKYALQRGLEYWGVDLISRNRSRTVRRTEEGQPESATMEKTSWDEIAIKSYTRAYHASENHLFWERQRRVRQHALALFKRAKERNRGDSTLPREPGHGLVAIAALACEAYAVGGRNLAETVVRGPTFGTARAVRDVEIASLGRYKTPSPKVAHGSPVKRDFLRARHRIVGLARAYYRPSDVVRGTSDAIAHVAGRNIGVAGGKPRAVEAVFTLPFVAYWEDVDRVVHCSSFQVSAPWNRDQRMKIAGVTTAAGTFSLHGGELKWAKPTSFYIRCSDTRRKFRPKGFGPMKRWRQWAKDLDRLVEQRASCVVRALQDDAALLETMERGQRYYDVFACAVTHATRGEADRLAGCSRCALTPCQEAHRVTTKPRGDAGVEQVQTSDCSLCEGKLVGPSKPRLHRTLTLLRQEHGLNYLRRLQREWESLEAVQVPTPYLRFKYARHLEVRSM

SEQ ID NO: 145 - protein, 499 aa, synthetic construct
TDSQSESVPEVVYALTGGEVPGRVPPDGGSAEGARNAPTGLRKQRGKIKISAKPSKPGSPASSLARTLVNEAANVDGVQSSGCATCRMRANGSAPRALPIGCVACASSIGRAPQEETVCALPTTQGPDVRLLEGGHALRKYDIQRALEYWGVDLIGRNLDRQAGRGMEPAEGATATMKRVSMDELAVLDFGKSYYASEQHLFAARQRRVRQHAKALKIRAKHANRSGSVKRALDRSRKQVTALAREFFKPSDVVRGDSDALAHVVGRNLGVSRHPAREIPQTFTLPLCAYWEDVDRVISCSSLLAGEPFARDQEIRIEGVSSALGSLRLYRGAIEWHKPTSLYIRCSDTRRKFRPRGGLKKRWRQWAKDLDRLVEQRACCIVRSLQADVELLQTMERAQRFYDVHDCAATHVGPVAVRCSPCAGKQFDWDRYRLLAALRQEHALNYLRRLQREWESLEAQQVKMPYLRFKYARKLEVSGPLIGLEVRREPSMGTAIAEM

SEQ ID NO: 146 - protein, 358 aa, synthetic construct
AGTAGRRHGSLGARRSINIAGVTDRHGRWGCESCVYTRDQAGNRARCAPCDQSTYAPDVQEVTIGQRQAKYTIFLTLQSFSWTNTMRNNKRAAAGRSKRTTGKRIGQLAEIKITGVGLAHAHNVIQRSLQHNITKMWRAEKGKSKRVARLKKAKQLTKRRAYFRRRMSRQSRGNGFFRTGKGGIHAVAPVKIGLDVGMIASGSSEPADEQTVTLDAIWKGRKKKIRLIGAKGELAVAACRFREQQTKGDKCIPLILQDGEVRWNQNNWQCHPKKLVPLCGLEVSRKFVSQADRLAQNKVASPLAARFDKTSVKGTLVESDFAAVLVNVTSIYQQCHAMLLRSQEPTPSLRVQRTITSM

SEQ ID NO: 147 - protein, 369 aa, synthetic construct
GVRFSPAQSQVFFRTVIPQSVEARFAINMAAIHDAAGAFGCSVCRFEDRTPRNAKAVHGCSPCTRSTNRPDVFVLPVGAIKAKYDVFMRLLGFNWTHLNRRQAKRVTVRDRIGQLDELAISMLTGKAKAVLKKSICHNVDKSFKAMRGSLKKLHRKASKTGKSQLRAKLSDLRERTNTTQEGSHVEGDSDVALNKIGLDVGLVGKPDYPSEESVEVVVCLYFVGKVLILDAQGRIRDMRAKQYDGFKIPIIQRGQLTVLSVKDLGKWSLVRQDYVLAGDLRFEPKISKDRKYAECVKRIALITLQASLGFKERIPYYVTKQVEIKNASHIAFVTEAIQNCAENFREMTEYLMKYQEKSPDLKVLLTQLM

SEQ ID NO: 148 - protein, 486 aa, synthetic construct
RAVVGKVFLEQARRALNLATNFGTNHRTGCNGCYVTPGKLSIPQDGEKNAAGCTSCLMKATASYVSYPKPLGEKVAKYSTLDALKGFPWYSLRLNLRPNYRGKPINGVQEVAPVSKFRLAEEVIQAVQRYHFTELEQSFPGGRRRLRELRAFYTKEYRRAPEQRQHVVNGDRNIVVVTVLHELGFSVGMFNEVELLPKTPIECAVNVFIRGNRVLLEVRKPQFDKERLLVESLWKKDSRRHTAKWTPPNNEGRIFTAEGWKDFQLPLLLGSTSRSLRAIEKEGFVQLAPGRDPDYNNTIDEQHSGRPFLPLYLYLQGTISQEYCVFAGTWVIPFQDGISPYSTKDTFQPDLKRKAYSLLLDAVKHRLGNKVASGLQYGRFPAIEELKRLVRMHGATRKIPRGEKDLLKKGDPDTPEWWLLEQYPEFWRLCDAAAKRVSQNVGLLLSLKKQPLWQRRWLESRTRNEPLDNLPLSMALTLHLTNEEAL

SEQ ID NO: 149 - protein, 400 aa, synthetic construct
AAVYSKFYIENHFKMGIPETLSRIRGPSIIQGFSVNENYINIAGVGDRDFIFGCKKCKYTRGKPSSKKINKCHPCKRSTYPEPVIDVRGSISEFKYKIYNKLKQEPNQSIKQNTKGRMNPSDHTSSNDGIIINGIDNRIAYNVIFSSYKHLMEKQINLLRDTTKRKARQIKKYNNSGKKKHSLRSQTKGNLKNRYHMLGMFKKGSLTITNEGDFITAVRKVGLDISLYKNESLNKQEVETELCLNIKWGRTKSYTVSGYIPLPINIDWKLYLFEKETGLTLRLFGNKYKIQSKKFLIAQLFKPKRPPCADPVVKKAQKWSALNAHVQQMAGLFSDSHLLKRELKNRMHKQLDFKSLWVGTEDYIKWFEELSRSYVEGAEKSLEFFRQDYFCFNYTKQTTM

SEQ ID NO: 150 - protein, 666 aa, synthetic construct
PQQQRDLMLMAANYDQDYGNGCGPCTVVASAAYRPDPQAQHGCKRHLRTLGASAVTHVGLGDRTATITALHRLRGPAALAARARAAQAASAPMTPDTDAPDDRRRLEAIDADDVVLVGAHRALWSAVRRWADDRRAALRRRLHSEREWLLKDQIRWAELYTLIEASGTPPQGRWRNTLGALRGQSRWRRVLAPTMRATCAETHAELWDALAELVPEMAKDRRGLLRPPVEADALWRAPMIVEGWRGGHSVVVDAVAPPLDLPQPCAWTAVRLSGDPRQRWGLHLAVPPLGQVQPPDPLKATLAVSMRHRGGVRVRTLQAMAVDADAPMQRHLQVPLTLQRGGGLQWGIHSRGVRRREARSMASWEGPPIWTGLQLVNRWKGQGSALLAPDRPPDTPPYAPDAAVAPAQPDTKRARRTLKEACTVCRCAPGHMRQLQVTLTGDGTWRRFRLRAPQGAKRKAEVLKVATQHDERIANYTAWYLKRPEHAAGCDTCDGDSRLDGACRGCRPLLVGDQCFRRYLDKIEADRDDGLAQIKPKAQEAVAAMAAKRDARAQKVAARAAKLSEATGQRTAATRDASHEARAQKELEAVATEGTTVRHDAAAVSAFGSWVARKGDEYRHQVGVLANRLEHGLRLQELMAPDSVVADQQRASGHARVGYRYVLTAM

SEQ ID NO: 151 - protein, 560 aa, synthetic construct
AVAHPVGRGNAGSPGARGPEELPRQLVNRASNVTRPATYGCAPCRHVRLSIPKPVLTGCRACEQTTHPAPKRAVRGGADAAKYDLAAFFAGWAADLEGRNRRRQVHAPLDPQPDPNHEPAVTLQKIDLAEVSIEEFQRVLARSVKHRHDGRASREREKARAYAQVAKKRRNSHAHGARTRRAVRRQTRAVRRAHRMGANSGEILVASGAEDPVPEAIDHAAQLRRRIRACARDLEGLRHLSRRYLKTLEKPCRRPRAPDLGRARCHALVESLQAAERELEELRRCDSPDTAMRRLDAVLAAAASTDATFATGWTVVGMDLGVAPRGSAAPEVSPMEMAISVFWRKGSRRVIVSKPIAGMPIRRHELIRLEGLGTLRLDGNHYTGAGVTKGRGLSEGTEPDFREKSPSTLGFTLSDYRHESRWRPYGAKQGKTARQFFAAMSRELRALVEHQVLAPMGPPLLEAHERRFETLLKGQDNKSIHAGGGGRYVWRGPPDSKKRPAADGDWFRFGRGHADHRGWANKRHELAANYLQSAFRLWSTLAEAQEPTPYARYKYTRVTM

SEQ ID NO: 152 - protein, 404 aa, synthetic construct
WDFLTLQVYERHTSPEVCVAGNSTKCASGTRKSDHTHGVGVKLGAQEINVSANDDRDHEVGCNICVISRVSLDIKGWRYGCESCVQSTPEWRSIVRFDRNHKEAKGECLSRFEYWGAQSIARSLKRNKLMGGVNLDELAIVQNENVVKTSLKHLFDKRKDRIQANLKAVKVRMRERRKSGRQRKALRRQCRKLKRYLRSYDPSDIKEGNSCSAFTKLGLDIGISPNKPPKIEPKVEVVFSLFYQGACDKIVTVSSPESPLPRSWKIKIDGIRALYVKSTKVKFGGRTFRAGQRNNRRKVRPPNVKKGKRKGSRSQFFNKFAVGLDAVSQQLPIASVQGLWGRAETKKAQTICLKQLESNKPLKESQRCLFLADNWVVRVCGFLRALSQRQGPTPYIRYRYRCNM

SEQ ID NO: 153 - protein, 392 aa, synthetic construct
ARNVGQRNASRQSKRESAKARSRRVTGGHASVTQGVALINAAANADRDHTTGCEPCTWERVNLPLQEVIHGCDSCTKSSPFWRDIKVVNKGYREAKEEIMRIASGISADHLSRALSHNKVMGRLNLDEVCILDFRTVLDTSLKHLTDSRSNGIKEHIRAVHRKIRMRRKSGKTARALRKQYFALRRQWKAGHKPNSIREGNSLTALRAVGFDVGVSEGTEPMPAPQTEVVLSVFYKGSATRILRISSPHPIAKRSWKVKIAGIKALKLIRREHDFSFGRETYNASQRAEKRKFSPHAARKDFFNSFAVQLDRLAQQLCVSSVENLWVTEPQQKLLTLAKDTAPYGIREGARFADTRARLAWNWVFRVCGFTRALHQEQEPTPYCRFTWRSKM

SEQ ID NO: 154 - protein, 455 aa, synthetic construct
MAKKGTNRKKMIVKVMKYELKYESGCADFNEMQNELWKLQRQTREVMNRTIQLCYHWSYVQADYCKQHGCARRDVKPCDVYETNATSLDGYIYQLFKDEYPNFLMANLIATLRKAHQKYDALLFDIQEGNSSIPSFKKDQPLIFSKEAIRLPECLSDKRQITLFCFSKPYKSAHPTLDKITFAVRARSASEKSIFDHIISGKYALGESQLVYEKKKWFFLLSYKFTPESVDVNPEKVLGVDLGVVNALCAGSVENPHDSLFIKGTEAIEQIRRLEARKRDLQKQARYPGDGRIGHGTKTRVSPVYQTRDAIARMQDTLNHRWSRALIDFACKKGYGTIQMEDLSGIKALESEKPYLKHWTYFDLQSKIIYKAEEKGIRVVKVNPKCTSRRCSACGYISKENRKNQVEFLCVNCGYHHNADYNAAQNLSIPQIDRLIEKQLKEQESEENEAGANPK

SEQ ID NO: 155 - protein, 410 aa, synthetic construct
MAKGTLSKVMKYELRYLDGCGDFQNMQKELWTLQRQSREILNRTIQIAYHWDYTDREQFKKTGQHLDIKAETGYKRLDGYIYDSLKEDVQNFASVNVNATIQKAWAKYKSSKIDVLRGDMSLPSYKSDQPLVLHAQSMKIFSSDDDDVLQVTLFSNAYKKACNYSNIRFIIGLHDATQRTIIKKVLSGDWGIGQSQIVYKRPKWFLYLTYNFSPEQHEVNPDKILGVDLGESIAIYASSIGEYGSLRIEGGEISAFAKQLEARKRSLQKQAAYCGKGRIGHGTKSRVSDVYKMEDKIANFRNTVNHRYSKMLIDYALKHMYGTIQMEDLSGIKKETGFPKFLQHWTYYDLQQKIEAKAKEHGINFIKVDPAFTSQRCSKCGNIDSENRPSQAVFCCKKCGYKTNADFNAS

SEQ ID NO: 156 - protein, 426 aa, synthetic construct
MNVTKVMRYQLIYQGGGGDFESLQNQLWEFQRQTRAILNKTIQTMYLATANQEKFSEKALYHDLCAEYPDMISSTVNATLREATKKYRSSVREILAGRMSLPSYKRDHPILLHNQSVALKQGNQGSYFATISVFSRKYQQGTPGVKQPSFQLIAKDNTQRTILQRLLSGEYKLGQCQLIYIRPKWFLNVAYSFTPSEKALDQEKVLGVDLGCVYAIYASSYGNHGIFKISGDEITSFERKQAAIQNRAFKNDLTRIREIEERRKQKLEQARYCGEGRIGHGVKTRVAPAYQDEGKISRFRETINHRYSKALVDYAEKNGYGTIQMEDLSGIKSSTGFPKRLQHWTYFDLQQKIKYKAEEQGIKVVKIKPAYTSQRCSRCGHIDPANRKSQSEFKCIACGFSSNADYNASQNISMRNIEKIIQGKAN

SEQ ID NO: 157 - protein, 432 aa, synthetic construct
MAKGTITKVMKYELRYLGGFSDFHEMQKEVWQLQRQYREILNKTIQIALHWDYVSAQQFGESGTYLDIREETGYKTLDGYIYNCLKGAYSEMASANLNAAVQKAWKKYKNSKTQVLQGVMSLPSYKSDQPILIDKGNVKLSAEENNGRAVLTLFSRNYRDTRGLKGNVEFSVLLHDGTQKSIFRNLIDKTYALGQCQLVYERKKWFLLLTYSFTPAGHALDPEKILGVDLGECYALYASSCYAPGILKIEGGEIAEYALRLEKRKRSLQQQARYCGEGRIGHGTKTRVGVVYKAEDRIASFRETINHRYSKELVDYAVSNGYGTIQMEDLSAIQKDLGFPKRLRHWTYYDLQMKITNKAKEHGIAVVKIDPRYTSQRCSKCGHIDPANRPRQEEFCCTACGYACNADYNASQNISIKGIEKIIQKMLSAKAD

SEQ ID NO: 158 - protein, 435 aa, synthetic construct
MSKGMLTKVMKYTLRYVGGCGDFHEMQSILWELQKQTRAVLNKTIQIAFEWDYRSREAFQETGEYLDVHAETGYKRLDGYIYNCLKNEYADFAGKNLNAAIQTAWKKYNQSKRDIQTGKMSLPSYRSNQPLIIHNDNVMISQDMQAAPSVRFTLLSLEYKKAHDLNTNPTFEVLINDGTQRAIFEKVRSGEYKLGQCMIQYDKKKWFLLLTYSFQPEKLTLDKNKILGVDLGETIVICASSVSERGRFVIDGGEITRFATQIEARKRSQQHQAAYCGEGRIGHGTKTRVDAVYKTEDRIANFRDTINHRYSRALVNYAVKHGFGTIQMEDLSGIKSSDDFPKFLRHWTYYDLQSKIESKAKERGIAVVKVNPRFTSRRCSKCGYIDEGNRKDQAHFCCLSCGFRANADFNASQNLSIKGIDKIIEKEYNANSKQT

SEQ ID NO: 159 - protein, 442 aa, synthetic construct
MGKPITKTMKYQIHYIDGCGDFHNMQKELWDLQRIVRQILNKTINESYLWFVRSEQYYRDTGENLSVEEQTGYKTLDGHIYNLLKQEYTQKLVSNSLNASIQAAYKKMKDSRRDVMIGTMSLPSYRSDQPIIIYNKNIKFSSHPEHGFVVDCSLFSDAYKKSQGYEKSVKFQVSVDDNTQRSIFENILTGNYKHGQCSIVYEKKKWFLLLTYSFVPEETKLDPDKILGVDVGVVYALYASSKGNHGTFKIKGDEAITFIQRVEARKHSRQLQGTYCGDGRIGHGTKTRVQPVYNERALISNFQDTINHRYSKALIDYAKKNGYGTIQMEDLSGIKEVQQYPKYLQHWTYYDLQLKIQYKAKEAGIGFVKVTPKYTSQRCSHCGNIDEANRPKQDVFRCTVCGYERNADYNASQNLSIKGIDRIIDDQLKQMNKANPKKTENA

SEQ ID NO: 160 - protein, 435 aa, synthetic construct
MSGGAITKVMKYDLTYKDGYGNFKDMQEAVWKLIRDTRTILNETIKIAYHWDYLNEKSKRETGEHLDLLEETGYKRLDGYIYDDLKDRFPDFASSNLNAAIQTAWKKYKQSQKDVYIGKMTLPSYKSDQPLPINKQSIKIYDEEREHIVELNLFSTKHKKEHGLASNVRFRINLHDNTQHAIYERVLSGEYTLGQCQLLYDRPKWFFILTYSFKPAQNKLDPDKILGVDMGETCALYASTFGEQGSFVINGGEVSEYAKREEARKRSLQKQAAVCGEGRIGHGTKTRVSSVYKEQERISNFRDTINHRYSKALIEYAVKNGCGTIQMEDLSGIRQSTDFPKFLRHWTYYDLQQKIKTKAKETGIAVSMIDPRYTSQRCSRCGHIDKANRKDQAHFHCLKCGYSCNADFNASQNISIRGIDKIIQKELGAKAKQTD

SEQ ID NO: 161 - protein, 431 aa, synthetic construct
MKEIAKVMKYQLIYLDGGGDFYELQQTLWDLQRQTREILNKTIQSMYLATATNTAFEENALYHRFGAEYPMMAALNVNATLRTAKKRYTSTIKETLRGTMSLPSYKRDQPILLHNQTIHLALEDGQYSALFSVYSEKFQKAHEGVARPRFALMARDGTQRAILDRLLDGSYRLGQSQMTYEQKKWFLSLTYKFVPEVRELDKSKILGVDLGCVYAIYASSMQQKGIFKISGDEITEFEKRQAAMQNREPVSTLERVEQLEQRRWQKQQQARYCGEGRVGHGTGTRVAPAYRDADKIARFRDTINHRYSKALVEYAEKNGFGTIQMEDLSGIKEDTGFPKRLRHWTYFDLQTKIQYKAAERGITVVKIDPQYTSQRCSRCGYIDKANRASQEKFLCQSCGFEANADYNASQNISVEKIDKLIAKDKKKLART

SEQ ID NO: 162 - protein, 436 aa, synthetic construct
MGQVTKVMRYQLIYQDGGGDFYTVQQELWELQRQTREILNKTIQTMYLADANKEKFDNAAERTLNRRFCVDHPDMYTKTVTATLRKAKAKYNASQKEILAGRMSLPSYKRDQPILLNPQGFKIEEESDSFFAAIAVFSDKYKNKHPDVDVKRLRFRLVVKDGTQRAIIRRVISGEYKLGRSQLLYSKKKWFLNVTYSFEPAEKKVDPDKILGVDLGCVYAIYASSFGSPGVFKISGDEVSSFERKQAAIQNRSPKSTLERVEKIEERHKQKQQQARYCGEGRIGHGTKTRIAPVYQDEDKIARFRDTVNHRYSKALIDYAEKNGYGTIQMEDLSGIKSATGFPKRLKHWTYYDLQTKIEYKAEERGIKVVKIDPRYTSQRCSRCGYIDSGNRKSQAEFCCMACGFSCNADYNASQNISIGGIAKIIADKRKEADAK

SEQ ID NO: 163 - protein, 368 aa, synthetic construct
YLDIREETGYKTLDGYIYNCLKGAYSEMASANLNAAVQKAWKKYKNSKTQVLQGVMSLPSYKSDQPILIDKGNVKLSAEENNGRAVLTLFSRNYRDTRGLKGNVEFSVLLHDGTQKSIFRNLIDKTYALGQCQLVYERKKWFLLLTYSFTPAGHALDPEKILGVDLGECYALYASSCYAPGILKIEGGEIAEYALRLEKRKRSLQQQARYCGEGRIGHGTKTRVGVVYKAEDRIASFRETINHRYSKELVDYAVSNGYGTIQMEDLSAIQKDLGFPKRLRHWTYYDLQMKITNKAKEHGIAVVKIDPRYTSQRCSKCGHIDPANRPRQEEFCCTACGYACNADYNASQNISIKGIEKIIQKMLSAKAD

SEQ ID NO: 164 - protein, 437 aa, synthetic construct
MAEKTIVKVMKFELRYIDGAGEFSEMQKHLWELQKQTREVLNKTIQMGYALECKRFAHHDKTGQWLDDKELTGSKYKAVADYINAELKEDYNIFYSDCRNSTVRKAYKKFKDAKNKIFSGEMSLPSYRSNQPIIIHNRNVIIRGNAESALVGLKVFSDGFKALHGFPAAVNFKLCVKDGTQRAIIENVISEIYKISESQLIYDNKKWFLILAYRFTQKKNDLNPDKILGVDLGVKFAVYASSIGEYGSFRIKGGEVTEFIKRLEKRKKSLQNQATVCGDGRIGHGTKTRVADVYKARDKISNFQDTINHRYSRAIVDYARKNGYGTIQLEKLDNSIEKKGDYSPVLVHWTYYDLRTKMEYKAAEYGIKVIAVEPKYTSQRCSKCGYISSENRKTQESFECIKCGYKCNADFNASQNLSVRDIDRIIDEYLGANPELT

SEQ ID NO: 165 - protein, 434 aa, synthetic construct
VVNVAKGALSKVMKFELSYLDGCGDFQNMQKELWTLQRQTREILNRTIQIAYHWDYTDREHFKKTGQHLDVKSETGYKRLDGYIYDELKETVQNFASVNVNATIQKAWAKYKSSKTDVLRGDMSLPSYKSDQPLVLHAQSIKLSEDKDGPVLQVTLFSNAHKKACDYSNVRFAFRLHDATQRAIFKNVLSGEYGLGQSQIVYKRPKWFLYLTYNFSPEQHGLDPDKILGVDLGESIALYASSLGDYGSLRIEGGEVTAFAKQLEARKRSLQKQAAHCGEGRVGHGTRARVSDVYKAEDKIANFRNTVNHRYSKKLIEYAIQNRYGTIQMEDLSGIKQDTGFPKFLQHWTYYDLQQKIEAKAKENGINFIKVDPSYTSQRCSKCGNIDSDNRPSQAVFCCTKCGFRANADFNASQNLSIPEIDKIIKKERGANTK

SEQ ID NO: 166 - protein, 455 aa, synthetic construct
MAKKGTNRKKMIVKVMKYELKYEKGCADFNEMQNELWKLQRQTREVMNRTVQLCYHWNYVQADYCKQHGCAHRDVKPCDVYETNATSLDGYIYQLFKDEYPNFLMANLIATLRKAHQKYDALLPDIQEGNSSIPSFKKDQPLIFSKEAIHLPECLSDKRQITLFCFSKPYKSAHPTLDKITFAVRAHSASEKSIFDNIINGKYALGTSQLVYEKKKWFFLLSYKFTPESVDVNPEKVLGVDLGVVNALCAGSVENPHDSLFIKGTEAIEQIRRLEARKRDLQKQARYPGDGRIGHGTKTRVSPVYQTRDAIARMQDTLNHRWSRALIDFACKKGYGTIQMEDLSGIKAMESEKPYLKHWTYFDLQSKIIYKAEEKGIRVVKVNPKCTSRRCSACGYISKENRKNQAEFLCVNCGYHHNADYNAAQNLSIPQIDRLIEKQLKEQESEESEAGANPK

SEQ ID NO: 167 - protein, 500 aa, synthetic construct
MTERHDNESSKIKAEVSLLNSSVPDFEKKRHVKVLKLHILKPAGDMKWDELGALLRDARYRVFRLANLAISEAYLDFHKWRSGGNEQPKLKISQLNRNLRSMLEDEVTGKQTKMIKSDRYSKSGALPDSIVSPLSMYKLGGLTSKSKWSEVLRGKSSLPTFKLNMAIPVRCDKPGDRRIERTKNGDAEVELRICLQPYPRVIIATGRNSLGDGQRAILDRLLDNTKYSEQGYRQRCFEIKEDQRSGKWHLFVTYDFPAIEPAKNLSRERIVGVDLGAACPLYAAINTGHARLGWKHFSPLAARVRALQNQTIRRRRQILRGGKVSLSEDSARSGHGRKRKLKPISKLEGKIDRAYTTLNHQLSATVIKFAKDNGAGVVQMEDLKGLRETLTGTFLGERWRYEELQRFIRYKADEAGIEIRLVNPQYTSRRCSECGHIHKDFTREFRDKSREGNKSVRFLCPDCGFTADPDYNAARNLASLDIAAIIERQLEIQGLRKHDP

SEQ ID NO: 168 - protein, 471 aa, synthetic construct
MKEKSKTLVKVARLRILKPAGDMKWSELGEMLRTVRYRVFRLANLAVSEAYLGFHMYRTNRATEFKAETIGKLSRRLREMLIEEGVDEKDLSRYSQTGAVPDTVAGALGQYKIRGITSPTKWRQVVRGQAALPTFRNDMAIPIRCDKQYQRRLEKTEAGEIEVELMICRKPYPRIVLGTADLGPGQRAILERLLQNTDNSADGYRQRLFEAKQDTQTKKWWLYVTYDFPRLKEGKLNQEIVVGVDLGFSIPLYVALNIGHARLGRRHFQALGNRIRSLQRQVLARRRSIQRGGRVNISHSTARSGHGRKRKLLPTEKLRGRIEKSYSTLNHQLSASVIDFAKNHHAGTIQIEDLANLKEELAGTFIGARWRYHQLQQFLKYKAEEAGITLNQVNPRYTSRRCSECGFINIDFDRAFRDAGRTEGRVTKFLCPECGYEADPDYNAARNISILDIDKLIRVQCKKQGLTYDAH

SEQ ID NO: 169 - protein, 475 aa, synthetic construct
MPERPKTVNKVIWFQIHKPAGDMTWKELGNLLREARYRVFRLANLAVSEKYLSFHMWRTGQEYKSETIGKLNRRLREMLIEEGVEEESQKRFSATGALPDTVVSTLAKGKLAAITSKSKWKDVVNGKTSLPTFKLNMAIPVRCDKAEQRRLRRTESGDVELELMICKQPYPRVVLKTGKLKSGQRAILDRLVENNDNSKEGYSQRVFEIKQVENNDGSKEWRLYISYTFPKKAVEANADVAVGVDIGFSVPLVAAVNNGLERLGYNDFRALNERIRSLQRQVLVRRRSMQSGGRDYVSTPTARSGHGRKRKLLPIQTLRKRWDNAYTTLNHQLSHAVVSFAENHGAATIQIENVKSLKDELRGTFLGQRWRYFELQQFLKYKADEVGIELREVNARYTSRRCSECGYINMAFTRQARDKGRVDGKPMEFVCPECGYKAHPDYNAARNIAMLDIEQKMQVQCKQQGITYADDSEVL

SEQ ID NO: 170 - protein, 448 aa, synthetic construct
MTWPELGNMLRTVRYRVFRLANLAVSEAYLGFHMFRTKRAEEFKAETMGKLSRRLREMLIEEGVDEKDLSRYSQTGAVPDTVAGALSQYKIRGITSPTKWRQIVRGQVALPTFRNTMSIPVRCDKLYQRRLEQGDSGEVEVELMICRNPYPRVVLGTGDLNPGQQAILERLLQNTDNSADGYRQRLFEIKEDVQTRKWWLYVTYDFPKTTGKLNPEIVVGVDLGFSIPLYVALNSGHARLGYLHFKALGERIKSLQKQVMARRRAIQRGGRVSISHSTARTGHGVKRKLQPTEKLRGRIEKSYSTLNHQLSASVIDFAKNHHAGVIQIEDLSGLKEQLTGTFIGARWRYHQLQQFLKYKAEEAGITLKQINPRYTSRRCSECGFINMDFDRAFRDAGRTYGKVTKFLCPECGYEADPDYNAARNIATLDIEKLIRVQCEKHGLKFDAH

SEQ ID NO: 171 - protein, 477 aa, synthetic construct
VGKEGKRNVKVMKIRILKPCDGMTWNELGQLLRDARYRVFRLANLTVSEAYLNFHLWRTGRSQEFKKQTIGQLNRQLRNILQQEKYDDEKLNRYSKTGALPDTVCSALWQYKLMAVMKKSKWSEVIRGKSSLPTFRNDMAIPVRCDKPEQKRIEKTEQGQVEAALQVCVQPYPRVILGTHTLGDGQDAILKRLLDNQNQAIGGYRQRSFEIKYDEQKRWWLFITYDFPATEVATDKTIAVGVDLGVSVPLYAAVNNGPARLGRREFGGLGRRIRDLRNQTDARRRSIQRSGREGQSDDTARAGHGRKRKLLPIHILEGRLDKAYTTLNHQMSAAVIKFAAEQGAGIIQIENLAGLQDELRGTFIGGRWRYRQLQDFLKYKTQEMGIELRQVNPKYTSRRCSKCGFIHKDFDRDYRNRHSENGKPAQFVCPNPDCKYESDPDYNAARNLATLDIEEQIRVQCQKQGLEYDSKKDKNAL

SEQ ID NO: 172 - protein, 472 aa, synthetic construct
MKEKSKTLVKVARLRILKPAGDMTWSELGEMLRTVRYRVFRLANLAVSEAYLGFHMFRTQRAAEFKAETMGKLSRRLREMLIEEGVDEKELNCYSLTGAVPDTVAGALHQYKIRGITSPTKWRQVVRGQAALPTFRNDMSIPIRCDKPYQRRLEKTEAGEVEVELMICRKPYPRIVLGTADVGPGQEVILERLLQNKDNSSDGYRQRLFEAKQDRQTGKWWLYVTYDFPRPEEGELNPEIVVGVDLGFSVPLYVAINNGYARLGRRHFQALGNRIRSLQRQVLARRRSIQRGGRVNISHDTARSGHGIKRKLLPTEKLRGRIEKSYSTLNHQLSASVIDFTKNHHAGTIQIEDLANLKEVLAGTFIGARWRYHQLQQFLKYKADEAGITLKEVNPRYTSRRCSECGFIHKDFDRAFRDSGRTDGKVARFVCPECGYGPVDPDYNAAKNISTLDIEKHIRVQCKKQGLEYEVH

SEQ ID NO: 173 - protein, 470 aa, synthetic construct
MKEKAKTLVKVARLRILKPAGDMTWPELGNMLRTVRYRVFRLANLAVSEAYLGFHMFRTKRAEEFKAETMGKLSRRLREMLIEEGVDEKDLSRYSQTGAVPDTVAGALSQYKIRGITSPTKWRQIVRGQVALPTFRNTMSIPVRCDKLYQRRLEQGDSGEVEVELMICRNPYPRVVLGTGDLNPGQQAILERLLQNTDNSADGYRQRLFEIKEDVQTRKWWLYVTYDFPKTTGKLNPEIVVGVDLGFSIPLYVALNSGHARLGYLHFKALGERIKSLQKQVMARRRAIQRGGRVSISHSTARTGHGVKRKLQPTEKLRGRIEKSYSTLNHQLSASVIDFAKNHHAGVIQIEDLSGLKEQLTGTFIGARWRYHQLQQFLKYKAEEAGITLKQINPRYTSRRCSECGFINMDFDRAFRDAGRTYGKVTKFLCPECGYEADPDYNAARNIATLDIEKLIRVQCEKHGLKFDAH

SEQ ID NO: 174 - protein, 468 aa, synthetic construct
MAKKAKTMFKVTNFRILKPAGDMTWKELGQLLRDARYRTFRMANLALSEAYLNFYLLKKGDLKEYKNVKIGQIAKRLRDMLIEEGVDEEVQNRFSPKVALPAYVYSALDQFKLRGLTSKSNWKKVLRGQASLPTFRLNMSVPIRCDKPEHRRLEKTENGNVEVDLMICRKPYPRVVLETLKLDGSSKAILDRLLENEDNSPGNYRQRCFEVKQNPRSNDWWLYVTYEMPVDKDKKLDPKVIVGVDLGFSVPLYVAINNGHARLGRRHFQALGKRIHNLQNQVLARRRSIQRGGQVNLSHSTSRSGHGRKRKLQPTEKLQQKINSAYSTLNHQLSSSVIDFANNHKAGTIQIEDLETLKEQLTGTYIGRQWRYYQLQQFIEYKAKENSITVKKINPKYTSRRCSMCGHIHADFDRTFRDRSSNKGFVTKFICPECNFEADPDYNAAKNISTLDIENKIKLQCKKQKIDY

SEQ ID NO: 175 - protein, 457 aa, synthetic construct
MPKITRKIELLFDRSGLSEEECKEKWRFIYQINDNLYRVANRLVNQLYLADEIDDILRLSDQEYIALRKKLANKKLDEATRISLEEQMSQVMKRVNERRSAILQRPQQSFAYSVVTDSDTEGLTAKILDVLKQDVLSHYKADTKEVLKGEKSISNYKKGMPIPFAFNDSLRLYKEDGFFYLKWYNGIRFLLNFGRDASNNQLIVERCLGISKDEISYKACSSSIQIKKKGNHSKIFLLLVVDVPVEQYAQKPNMVVGVDLGLNVPIYAASNSTLERKAIGSREAFLNQRGAFQRRFRALQRLQTTKGGRGRLHKLEPLERVREAERNWVRTQNHLFSREVINFAIDVGASTIQMEKLANFGRDAQGEVREDKKYVLRNWSYFELQNLIEYKAKRAGIKVKYINPAFTSQTCSECGQLGERDSIHFKCTNPDCPNCGKDIHADYNGARNIAKSKDYIK

SEQ ID NO: 176 - protein, 468 aa, synthetic construct
MPTITRKIELTLLTEGLSEEQRKEQWGLLYHINDNLYKAANNISSKLYLDDHVSSMVRMKHAEYLSLLKELARAEKQKTPDADAIAELRKKVAAAEKEMTDQEHAICKYATEMSTQSLSYRFATELETNIFAKILDCLKQGVFATFNSDARDVKRGERAIRNYKKGMPIPFAWDKSLRIEKDNKDFYLRWYNGLRFLFNFGKDRSNNRLIVERCLKMDADYDGEYKLCNSSIQIAKREGKTKLFLLLVVKIPQEHVELNKKVVVGVDLGINVPAYVATNITEERKAIGDREHFLNSRMAFQRRYKSLQRLRGTAGGKGRAKKLEPLERLRKAEHNWVHTQNHLFSREVVDFAVKSHAATIHMEDLSGFGKDNDGNADERKEFVLRNWSYYELQNMIAYKAAKYGIKVEKIHPAYTSKTCSWCGQLGFREGVTFICENPECKQCGEKVHADYNAARNIANSKDIIKKNE

SEQ ID NO: 177 - protein, 468 aa, synthetic construct
MPTITRKIELTLLTEGLSEEQRKEQWGLLYHINDNLYKAANNISSKLYLDDHVSSMVRMKHAEYLSLLKELARAEKQKTPDADAIAELRKKVAAAEKEMTDQEHAICKYATEMSTQSLSYRFATELETNIFAKILDCLKQGVFATFNSDARDVKRGERAIRNYKKGMPIPFAWDKSLRIEKDNKDFYLRWYNGLRFLFNFGKDRSNNRLIVERCLKMDADYDGEYKLCNSSIQIAKREGKTKLFLLLVVKIPQEHVELNKKVVVGVALGINVPAYVATNITEERKAIGDREHFLNSRMAFQRRYKSLQRLRGTAGGKGRAKKLEPLERLRKAEHNWVHTQNHLFSREVVDFAVKSHAATIHMEDLSGFGKDNDGNADERKEFVLRNWSYYELQNMIAYKAAKYGIKVEKIHPAYTSKTCSWCGQLGFREGVTFICENPECKQCGEKVHADYNAARNIANSKDIIKKNE

SEQ ID NO: 178 - protein, 468 aa, synthetic construct
MPTITRKIELTLLTEGLSEEQRKEQWGLLYHINDNLYKAANNISSKLYLDDHVSSMVRMKHAEYLSLLKELARAEKQKTPDADAIAELRKKVAAAEKEMTDQEHAICKYATEMSTQSLSYRFATELETNIFAKILDCLKQGVFATFNSDARDVKRGERAIRNYKKGMPIPFAWDKSLRIEKDNKDFYLRWYNGLRFLFNFGKDRSNNRLIVERCLKMDADYDGEYKLCNSSIQIAKREGKTKLFLLLVVKIPQEHVELNKKVVVGVNLGINVPAYVATNITEERKAIGDREHFLNSRMAFQRRYKSLQRLRGTAGGKGRAKKLEPLERLRKAEHNWVHTQNHLFSREVVDFAVKSHAATIHMEDLSGFGKDNDGNADERKEFVLRNWSYYELQNMIAYKAAKYGIKVEKIHPAYTSKTCSWCGQLGFREGVTFICENPECKQCGEKVHADYNAARNIANSKDIIKKNE

SEQ ID NO: 179 - protein, 468 aa, synthetic construct
MPTITRKIELTLLTEGLSEEQRKEQWGLLYHINDNLYKAANNISSKLYLDDHVSSMVRMKHAEYLSLLKELARAEKQKTPDADAIAELRKKVAAAEKEMTDQEHAICKYATEMSTQSLSYRFATELETNIFAKILDCLKQGVFATFNSDARDVKRGERAIRNYKKGMPIPFAWDKSLRIEKDNKDFYLRWYNGLRFLFNFGKDRSNNRLIVERCLKMDADYDGEYKLCNSSIQIAKREGKTKLFLLLVVKIPQEHVELNKKVVVGVDLGINVPAYVATNITEERKAIGDREHFLNSRMAFQRRYKSLQRLRGTAGGKGRAKKLEPLERLRKAEHNWVHTQNHLFSREVVDFAVKSHAATIHMQDLSGFGKDNDGNADERKEFVLRNWSYYELQNMIAYKAAKYGIKVEKIHPAYTSKTCSWCGQLGFREGVTFICENPECKQCGEKVHADYNAARNIANSKDIIKKNE

SEQ ID NO: 180 - protein, 463 aa, synthetic construct
MPTMTRKIELKLCTEGLSDEERKAQLGLLYHINDNLYKAANNISSKLYLDDHVSSMVRLKHAEYLSLLNEFEKAKKKGDEEQIVELSLRVAAAEKELTDQELAICKYATEMSTDTLAYRFANEIEINVFGQILACLKQGIHSTFKKDAADVKRGERAIRNFKKGMPIPFPWSKSIRIENEGSDFYLRWYNGLRFRFDFGKDRSNNRLIVSRCLNLDPDFEDEYKLSNSSLQMVKRDGRPKLFLLLVVNIPQENVELNKKIVVGVDLGINSPAYVATNITMERQRIGSRDTFLNARMAIQRRFQSLQKLQNTAGGRGRKKKLEPLERLKETERNWVRTQNHLFSRDVVQFAVKTRAATIHMEDLSGFGKDDDGNADEKKEFVLRNWSYYELQTMIKYKAAKYGIKVEKIRPAYTSRTCSWCGHEGDRKGETFICENPECEKYGKKENADYNAARNIANSTDIIK

SEQ ID NO: 181 - protein, 468 aa, synthetic construct
MPTITRKIELTLCTEGLSDEQRKEQWGLLYHINDNLYKAANNISSKLYLDEHVSSMVRMKHAEYLSLLKELARAEKQQTPDEGLIAELSRKLSAAEKEMADQELAICKYATEMSTQTLSYNFAKEIETNIFGQILTCLRQGVYATFNSDAKDVKRGERAIRNYKKGMPIPFPWNNSLKIESDSGEFYLRWYNGLRFLLTFGKDRSNNRMIVNRCMKMDEDFEGEYKLCNSSIQLAKRDGKPKLFLLLVVNIPQEHVKLNKKIVVGVDLGVNVPAYVATNITEERKAIGDREHFLNTRMAFQRRYKSLQRLKGTAGGKGRTKKLEPLERLRDAERNWVHTQNHLFSREVVNFAVQARAATIHMEDLSGFGKDKDGNADEKKEFVLRNWSFYELQNMIAYKSAKYGIKVVKIRPAYTSKTCSWCGQQGDRKSTTFICENPKCKHYGESIHADYNAARNIANSNDIVKENE

SEQ ID NO: 182 - protein, 467 aa, synthetic construct
MPKITRKIEMTLCTEGLSDEQRKEQWGLLYHINDNLYKAANNISTKLYLDEHVSSMVRMKHADYLSLLKELAKAEKKSPDEDLIAELREKLAAAEQEMTDQELAICKYATEMSTQTLAYKFATEIEINVFGQILACLKQAAQSNFKSDAKDVKRGERAIRNYKKGMPIPFPWNDNIRIDADGDEFYLRWYNGLRFHLTFGKDKSNNRMIVKRCLKMDKDFEGEYKLCNSSIQMVKRDGKPKLFLLLVVNIPQEHVELNKNVVVGVDLGVNVPAYVATNITEERKAIGEREHFLNTRMQIQRRYKSLQRLKATAGGKGRTKKLEPLERLRKAEHNWVHTQNHLFSREVVNFAVQTHAATIHMEDLSGFGKDDDGNADEQKEFVLRNWSFYELQNMIAYKAAKYGIKVEKVKPAYTSKTCSWCGQLGFRQGVTFICENPACKQCGEKVHADYNAARNIANSKDIIKKNE

SEQ ID NO: 183 - protein, 468 aa, synthetic construct
MPTITRKIELTLLTEGLSEEQRKEQWGLLYHINDNLYKAANNISSKLYLDDHVSTMVRMKHAEYLSLLRELARAEKQKKPDVDAIAELREKVTAAEKEMSDQERAICTYATEMSTQSLSYRFATEIETNIFAKILDCLKQGVFATFNSDARDVKRGERAIRNYKKGMPIPFAWDKSLRIEKDNKDFYLRWYNGLRFLFNFGKDRSNNRLIVERCLKMDADYDGEYKLCNSSIQIVKREGKVKLFLLLVVSIPQEHVELNKKIVVGVDLGINVPAYVATNITEERKAIGDREHFLNSRMAFQRRYKSLQRLKGTAGGKGRTKKLEPLERLRKAEHNWVHTQNHLFSREVVDFAVKSHAATIHMEDLSGFGKDNDGNADERKEFVLRNWSYYELQNMIAYKAAKYGIKVERIRPAYTSKTCSWCGQLGFREGVTFICENPECKQCGEKVHADYNAARNIANSKDIIKKNE

SEQ ID NO: 184 - protein, 491 aa, synthetic construct
TPFVLYFQNYSLSLRQHITLYSMPTITRKIELTLCTEGLSDQERKDQWNLLYHINDNLYRAANNISSKLYLDDHVGSMVRLKHAEYLSLLRAMEKAKKQKAPDEEVIAELSQQVAAAEQEMDEQAKAICQYATEMSTQTLSYRFATELETNIFGQILTCLRQGVFSTFNSDARDVKRGERSIRTYKKGMPIPFPWNDSLRIGFEDGEFYLRWYNGLRFRFDFGKDRSNNRLIVQRCMKMDKDYEGDYKLCNSSIQMVKREGKPKFFLLLVVNIPQERVELNKNIVVGVDLGINAPAYVATNTTPERKQIGDREHFLNERMAFQRRFKSLQRLKGTTGGRGRAKKLEPLERLRKAEQNWVHTQNHLFSREVIDFAVKARAATIHMEDLSGFGKDRDGNADERKEFVLRNWSYYELQNMITYKAAKYGIKVEKIRPAYTSKTCSWCGHQGFREGITFICENPECKKFGEKEHADYNAARNIANSKEIIKNNEE

SEQ ID NO: 185 - protein, 469 aa, synthetic construct
MPTITRKIELHLCTEELSDEQQKAQRLLLYHINDNLYKAANNVSSKLYLDEHVSSMVRLKHDEYLSLLRELARAEKKADDELATQLREKLVAAEREMTDQELAICKYATEMSTQSLSYRFVTELETKIFAKILDCLKQGVYATFNSDSRDVKRGERAIRNYKKGMPIPFAWDKSVRIEYEEKEKDFFLRWYNDIRFKFHFGRDRSNNRLIVSRCMKLDKDYEGDYQLCNSSIQIVKRDGSTKYFLLLVVKIPQEHVELNKKIVVGVDLGINYPAFAATNCTEERMSIGDREHFLNTRMQFQRRFKSLQRLKGTTGGKGRNKKLEPLERLRKAEHNWVHTQNHLFSLKVVNFAVQAHAATIHLEDLSGFGKDDDGNADERKEFVLRNWSYYELQNMIKYKAKKFGIQVEKIRPAYTSQTCSWCGQRGFRQGITFICENPECKKCGEKENADYNAARNIANSKDIIKDKDE

SEQ ID NO: 186 - protein, 460 aa, synthetic construct
MPIITRKIELHISKEGLSAEDYKAQWQYLRQINDNLYMAANRVSSHCFLNDEYKYRLCLQIPDYIDIEKQLKDSKRARLSKEELGQLKKRKKELENTVKGRFQDEFEKNSLYTIISNEFGEIIPGQILTCLRQCVQSKYNRAKEELEKGERAISTYKKGMPIPFPINKSIRLQKQGEDFVLKWYNKIVFKLHFGRDRSNNRVIVERLIQSALNDKQKGEDYVMNNSSIQLVEKDKMTKIFLLLSMDIPTQKRKLDSELVLGVDLGLNFPLYYATNQSANIHDHIGDKDIFLKERMVFQRRFKELQRLQCTQGGRGRKKKLEPLEKLRDKERNWVRTKNHIFSREVIKVALHLGAGTIHLENLHNFGKDGNGELKNSKKFVFRNWSYFELQSMIEYKAKMEGITVKYVNPAYTSQTCSVCGMIGERKEQAVFRCMNSSCLEYGKEVNADFNAARNIAKAKM

SEQ ID NO: 187 - protein, 466 aa, synthetic construct
MPTITRKIELTLCTDGLSDDLRKDQWQLLYHINDNLYKAANNISSKLYLDEHVASMVRLKHAEYLGLIKELAKARKRADDEAVRDLCSKLAVAEQEMNEQAKAICDYATEMSTQTLSYNFAKEIETNIFGQILTCLRQGVLLNFNSDARDVKRGERAIRNYKKGMPIPFPWNDTIKIVSEGDEFYLRWFSGLRFHLNFGKDRSNNRMIVRRCLKMEQDFDEEYKISNSSIQVAKRDGKQKLFLLLVVQIPQEQVVLNKKIVVGVDLGVNVPAYVATNITEERKAIGDREHFLNTRMQFQRRYKSLQRLKTTEGGRGRAKKLEPLERLRKAEHNWVHTQNHLFSREVVNFALQTQAATINMEDLSGFGKDNDGNADECKEFVLRNWSYYELQNMIVYKASKYGIRVQKIRPAYTSKTCSWCGHMGFREGVTFICENPDCKQFGEKVHADYNAARNIANSKEIIKNDE

SEQ ID NO: 188 - protein, 444 aa, synthetic construct
MSKTVTKTVKIALICEHTNKYGEKVDYKDINKLLWKLQKQTRELKNKTIQLCWEYNNFSCDYYKEHHEYPNMEDILKYKRINGFVENKLKTVNDLYSSNCSTTILSTCNEFQNYRSEFLKGTRSINSYKSDQPLDLHKGAIKLEHDGKDFYVSLKLLKRSAFNAMEFKGSDIRFKLNVKDKDKSTLKILESCYDKIYSISASKMTYDRKAGKWFLLLAYSFTPAKTENLDPEKILGVDLGIKIPICASVYGDLDRLTIEGGKIEEFRRRVEARKRSLQKQGKQCGDGRIGHGTKKRIKPITDIGDKIARFRDTENHIYSRYLIEYAVKKGCGTIQMEKLEGITREKDIFLKNWTYFDLQKKIEYKAKEKGIKVVYIEPAYTSKRCSSCGFIDTDNRLDQAHFKCLKCGFNENADYNASQNIGIKNIDKIIKEEHKSASDKLTSE

SEQ ID NO: 189 - protein, 439 aa, synthetic construct
VIILTKVVKLYLISEQINKEGQKIDYQRINSILWDLQKQTRDIKNRTVQLCWEWMNFSSDYCKTQEEYPKERDILGYTLEGYVYDYFKTGYDLYTGNISTSSREVCSSFKNVKKEILKGERSILSYKANQPLDLHKKAISLEYDNFNFFVKLKLLNRTGKKKYDITEDINFKIQVNDKSTRTILERCYDKEYKISGSKLIYEKKKKLWRLNLCYSFENSQVETLEKDKILGIDLGIVYPLMASIYGEYDRFSIKGGEIEEFRRRTEARKRSILQQTKYCGDGRIGHGRNKRTQPAYKINDKIARFRDTANHKYSRALIEYAVKKNCGIIQMENLTGISDNTDCFLKDWSYYDLQTKIENKAKEMGIKVVYIKAQYTSQRCSRCGYIDVNNRIRQALFKCQNCGYETNADYNASQNIGMYDIENIIEETLKIQSANVKQS

SEQ ID NO: 190 - protein, 437 aa, synthetic construct
MTKVTKVYLISEQIDKDGNKIDFKKISELLWNLQMQTRDIKNKCVQLCWEWLNFSSDYYKKSEEYPKEKDTLGYTLSGFVYDRIKNGSDLYSSNLSTSSRDTCTAFSNYKKEMLKGERSVLSFKANQPLDIHNKAIKLSYENGNFFVALKMLNRAGKEKYGIKDDLRFRMQVRDKSVRTILERLMNDEYKVSASKLMYDKKKKLWKLNLCYSFDNHVISTLDTEKIMGVDLGVVYPIMASVNGDYARFSIKGGEIEAFRSRVEARRRSLLNQSRYCGDGRIGHGRKKRTEPATQIADKIARFRDTTNHKYSRALIDYAIKNGCGTIQMEKLTGITSSAEHFLKEWSYFDLQTKIESKAKEAGIKVVYINPKFTSQRCNKCGYIHTDNRPVQARFCCQKCGYEENADYNASQNIGTKHIDVIIEETLKMQCEPETPTE

SEQ ID NO: 191 - protein, 428 aa, synthetic construct
MNKVVKLALICEQSDKDNSPVDYKKINEILWELQKQTREIKNKAIQYCWEYNNFSSDYYKKFNEYPKEKDILSYTLVGFVNDKFKTGNDLYSGNCSTTVRNACTEFKNSKKELIKGSRSIINYRSNQPLDIHNKCIRIEFENNCFYTYLKLLNRPAFKKYNFANTEIKFKILVRDNSTKTILERCISNEYEIAASKLLYDQKKKCWFLNLVYAFEIKSNNSLDPNKILGVDLGIHYPICASVYGSLDRFTIDGGEIDEFRRRVESRKISMLKQGKNCGDGRIGHGIKARNKPVYNIEDKIARFRDTANHKYSRALIEYAVKHTCGTIQMEDLTGITDIANRFLKNWSYYDLQTKIEYKAKEAGINIVYIDPKNTSRRCSKCGYIDKENRETQSRFICLKCGFKENADYNASQNIGIKDIDKLIKEDVH

SEQ ID NO: 192 - protein, 443 aa, synthetic construct
VTLLVKVVKIYLISEQFDKAGNQIDYKEVNKILWELQKQTREAKNKTVQLLWEWNNFSSDYVKASGIYPKAKDIFGYSSVHGQANKELRTKLALNSSNLSTTTMDVCKIFNTYKKEVWEGKRSVPSYKSDQPLDLHKESIKLIYENNEFYVRLALLKKAEFAKYGFKDGFRFKMQVKDNSTKTILERCFDEVYKINASKLLYDQKKKKWKLNLSYSFDNKNISELDKEKILGVDVGVNCPLVASVFGDRDRFIIKGGEIEKFRKSVEARRRSMLEQTKYCGDGRIGHGRKKRTEPALNIGDKIARFRDTTNHKYSRALIEYAVKKGCGTIQMEKLTGITSKSDRFLKDWTYYDLQTKIENKAKEVGINVVYIAPKYTSQRCSKCGYIHKDNRPNQAKFRCLECDFESNADYNASQNIGIKNIDKIIEKDLQKQESEVQVNENK

SEQ ID NO: 193 - protein, 428 aa, synthetic construct
MNKVVKLALICEQSDKNNSPVDYKKVNEILWELQKQTREIKNKTIQYCWEYYNFSSDYYKKFNKYPKEKDILSYTLWGFINDKFKTGNDLYSGNCSATTKKVIKEFKNSKKELIRGSRSIINYKSNQPLNIHNKCIHLQFKNNNFYVSINLLNRRSFKKYNFANTAIKFKILVRDNSTKAILERCISNEYKISESQLIYNKKKKCWFLNLSYAFEIKSNNSLDPNKILGVDLGIHYPICASVYGSLDRFTIDGGEIDEFRRRVESRKISMLKQGKNCGDGRIGHGIKARNKPVYNIEDKIARFRDTANHKYSRALIEYAVKNNCGTIQMEDLTGITDNANRFLKNWSYYDLQTKIEYKAKEASINVVYINPENTSRRCSKCGYIDKENRKTQSSFICLKCGFKENADYNASQNISIKDIDKLIKEDVH

SEQ ID NO: 194 - protein, 443 aa, synthetic construct
VTLLVKVVKIHLISEQFDKAGNRIDYEEVNKILWELQKQTREAKNKTVQLLWEWNNFSSDYVKASGIYPKAKDIFGYSSVHGQANKELRTKLALNSSNLSTTTMDVCKNFNTYKKEVWKGKRSVPSYKSDQPLDLHKDSIKLIYENNQFYVRLALLKKAEFAKYGFKDGFHFKMQVKDNSTKTILERCFDEVYKINASKLLYDQKKKKWKLNLSYSFDNKNISELDKEKILGVDVGVSYPLVASVFGDRDRFKIKGGEIEKFRKSVEARRRSMLEQTKYCGDGRIGHGRKKRTEPALNIGDKIARFRDTTNHKYSRALIEYAVKKGCGTIQMEKLTGITSKADRFLKDWTYYDLQTKIENKAKEVGINVVYIAPKYTSQRCSKCGYIHKDNRPNQAKFRCLECDFESNADYNASQNIGIKNIDKIIEKDLQKQESEVQVNENK

SEQ ID NO: 195 - protein, 449 aa, synthetic construct
LIWKDALGGIILTKIVKLYLISEQIDKDGNRVDYKEINSILWNLQKQTRDIKNKTVQLCWEWMNFSSDYYKKNELYPNEKEILNLTLRGYAYDHFKQGYDLYSSNISVLTEAVCGAFKNAKKEMLNGEKSVLSYKAEQPLDIHKKCIKLEYDKNFYVKLKMLNKAGKKKYGIEDDLNFKIQVEDKSTRTILERCIDGEYVVSGSKLIYDKKKKLWKLNLCYSFKANEIESLDKNKILGIDLGIACPLMASVNGEFDRFSIKGGEIETFRKRIEARKRSVLHQTKYCGDGRIGHGRNKRTEPAYKINDKIARFRDTANHKYSRALIDYAIRKNCGMIQMENLTGISDKKEHFLKEWSYYDLQTKIENKAKEKGIKIVYINPEYTSQRCSKCGYIDANNRELRAVFKCQKCGFEADADYNASQNIGIKNIEDIIENTLKISSANEKQTKNT

SEQ ID NO: 196 - protein, 462 aa, synthetic construct
VFYSTFLCYILTKYIDFSANECYNINTSSEVKQLMNKVVKLALICEQSDKDNSPVDYKKINEILWELQKQTREIKNKAIQYCWEYNNFSSDYYKKFNEYPKEKDILSYTLVGFVNDKFKTGNDLYSGNCSTTVRNACTEFKNSKKELIKGSRSIINYRSNQPLDIHNKCIRIEFENNCFYTYLKLLNRPAFKKYNFANTEIKFKILVRDNSTKTILERCISNEYEIAASKLLYDQKKKCWFLNLVYAFEIKSNNSLDPNKILGVDLGIHYPICASVYGSLDRFTIDGGEIDEFRRRVESRKISMLKQGKNCGDGRIGHGIKARNKPVYNIEDKIARFRDTANHKYSRALIEYAVKHTCGTIQMEDLTGITDIANRFLKNWSYYDLQTKIEYKAKEAGINIVYIDPKNTSRRCSKCGYIDKENRETQSRFICLKCGFKENADYNASQNIGIKDIDKLIKEDVH

SEQ ID NO: 197 - protein, 430 aa, synthetic construct
LISEQIDKDGNRVDYKEINSILWNLQKQTRDIKNKTVQLCWEWMNFSSDYYKKNELYPNEKEILNLTLRGYAYDHFKQGYDLYSSNISVLTEAVCGAFKNAKKEMLNGEKSVLSYKAEQPLDIHKKCIKLEYDKNFYVKLKMLNKAGKKKYGIEDDLNFKIQVEDKSTRTILERCIDGEYVVSGSKLIYDKKKKLWKLNLCYSFKANEIESLDKNKILGIDLGIACPLMASVNGEFDRFSIKGGEIETFRKRIEARKRSVLHQTKYCGDGRIGHGRNKRTEPAYKINDKIARFRDTANHKYSRALIDYAIRKNCGMIQMENLTGISDNKEHFLKEWSYYDLQTKIENKAKEKGIKIVYINPEYTSQRCSKCGYIDANNRELRAVFKCQNCGFEADADYNASQNIGIKNIEDIIENTLKISSANEKQTKNT

SEQ ID NO: 198 - protein, 429 aa, synthetic construct
LVKVVKIYLISEQVDEQGKDVDYNTICGVLWDLQWETREIKNKTVQLCWEWSGFSSDYYKKYGEYPKEKNLLDYTMGGFVYDKLKSKYHLYTANLSTTSQNTCGIFRTYKVDFVKGNRSVLSFKADQPLDVHKKSISIDRIDDNYFVKLKLLNKSGIQKYGIRDDFHFRMLVKDNSTKTILERCVGGDYKAAASKIIYDKKKKMWCLNLSYEFDVNTAKDLNKNRILGIDIGIVYPVVASVNGELDRFVIQGGEIETFRRRVENRKKSLLKQTKYCGDGRIGHGRNKRTEPVDIISDQIARFRNTANHKYSRAVIDYAVRKQCGTIQMENLKGITDKSDRFLKNWSYYDLQQKIEYKAKEKGINVVFINPKYTSQRCSRCGYIDSANRPKLPNQSKFLCIKCGFTENADYNASQNIALYNIEKLIDAEA

SEQ ID NO: 199 - protein, 1120 aa, synthetic construct
MEDDKKTKESTNMLDNKHFWAAFLNLARHNVYITVNHINKILEEGEINRDGYETTLKNSWDEIKDINKKDRLSKLIIKHFPFLEAATYRQNPTDTTKQKEEKQAEAQSLESLKKSFFVFIYKLRDLRNHYSHYKHSKSLERPKFEEDLQEKMYNIFDVSICLVKKDYQYNKDINPDEDFKHLDRKRKGKFKYSFADNEGNITESGLLFFVSLFLEKKDAIWVQKKLEGFKCSNESYQKMTNEVFCRSRMLLPKLRLESTQTQDWILLDMLNELIRCPKSLYERLRGDDREKFRVPLDPIDEDYDAEQEPFKNTLVRHQDRFPYFALRYFDYNEIFTNLRFQIDLGTYHFSIYKKLIGGQKEDRHLTHKLYGFERIQEFDKQNRPDEWKAIVKDSDTFKKKEEKEEEKPYISETTPHYHLENKKIGIAFKNHNIWPSTQTELTNNKRKKYNLGTSIKAEAFLSVHELLPMMFYYLLLKTENTKNDNKVEGKKETKKQEKHKIESIIESKIKDMYALYDAFANGEINSEKELEEYLKGKDIEIVHLPKQMIAILKNEHKDMAEKAEAKQEKMKLATENRLKTLDKQLKGKIQNGKRYNSAPKSGEIASWLVNDMMRFQPVQRDENGEPLNNSKANSTEYQLLQRTLAFFGSEHERLAPYFKQTKLIESSNPHPFLNDTEWEKCSNILSFYRSYLKAKKNFLESLKPEDWEKNQYFLMLKEPKTNRKTLVQGWKNGFNLPRGIFTEPIRKWFMEHWKNIKVDDSKRVGLVAKVIPLFFSEKYKDSVQPFYNYPFNVGDVNKPKEEDFLHREERIELWDKKKDKFKGYKAKKKFKEMTDKEKEEHRSYLEFQSWNKFERELRLVRNQDIVTWLLCTELINKLKIDELNIKELKKLRLKDINTDTAKEEKNNILNRVMPMELPVTVYEVNKGGYIIKNKPLHTIYIKEAKTKLLKQGNFKALVKDRRLNGLFSFVKTPSEAESESNPISKSRVESELGKYQNARLNIIEDMLALEKTLIDKYNSLDTDNFHNMLTGWLKLKGKANKARFQNEVDLLVAVRNAFSHNQYPMYNENLFGNIERFSLSSSNIIESKGLDIAVKLKEEVSKAAKKIQNEEDNKKEKETF

SEQ ID NO: 200 - protein, 1098 aa, synthetic construct
MENDKKLEESACYTLNDKHFWAAFLNLARHNVYITVNHINKTLGEGEINRDGYETTLENTWNEIKDINKKARLRELIIKHFPFLEAATYQQRSTDSTKQKEEKQAEAQSLESLKHCLFPFLKKLQKSRDHYSHYKHSKSLERPKFEEDLQKKMYNIFDVSIRLVKEDYKHNTDINLKEDFKHLNRTGKFKYSFADNKGNITESGLLFFISLFLEKKDAIWMQKKLKGFKDSREKYQKMTNEVFCRSRILLPKLRLESTQTQDWILLDMLNELIRCPKSLYERLREKERKEFKVPIEIADEDYDAEQEPFRNTLVRHQDRFPYFALRYFDYNEIFTNLRFQIDLGTYHFSIYKKLIGGNKEDRHLTHKLYGFERIQEFAKQNRPDEWKALVKDLDTFNKEEEKLYISETTPHYHLENEKIGIVFKNHNIWPSTQTELTNNNRKKYNLGVSIKAEAFLSVHELLPMMFYYLLLKTKNTHNGNEVEAKKKGTKNKKQEKHKIEAIIESKIKDIYNLYDAFANGEINSIEELEEHCKGKDIEIGHLPKQMIAILKDEHKDMAKKAETKQEKMILATENRLKTLDKKLKGKIRNGKRCNSALKSGEIASWLVNDMMRFQPVQRDEEGKPLNNSKANSTEYQLLQRTLAFFGSEHERLDPYFKQMNLIGDNNPHPFLNDTEWEKCNNILSFYRSYLKARKNFLESLKPEDWEKNQYFLMLKEPKTNRKTLVQGWKNGFNLPRGIFTEPIRKWFMEHWKSIKVDDLKRVGLVAKVIPLFFSEKYKDSVQPFYNYPFNIGNINKPEEKDFLDYEKRKKLWDKKQAKFKGKTDKEKREDPSYPDFKSWKKFERELRLVRNQDIVTWMLCMELFEEQKVDGLNTGQIHLKDIVTNVDEEGNNNILNQIKPMQLPITVYDKENSPKRKPLHTIYIEETGTKLLKQGNFKALVKDRRLNGLFSFVKTSSEAESKSKPISKLRVEYELGAYQKARIDIIKDMLALEKTLIDNDENLPTNKFSDMLKSWLKGKGEANKARLQNDVDLLVAVRNAFSHNQYPMYNSEVFKGMKLLSLSSDIPEKEGLGIAKQLKDKIKETIERIIEIEKEIRN

SEQ ID NO: 201 - protein, 1091 aa, synthetic construct
MKEEEKGKTPVVSTYNKDDKHFWAAFLNLARHNVYITVNHINKILGEAEINIDGYDISLKNIWSGIRDINKKARLEELMIKHFPFLEAMTHQQRLTGNTKQKEEKQAKIQSFEILKDNLFLLLKMLQDLRNHYSHYKHSKSLERPKFDEDIRNKLYNIFDTSIRLVKEDYKHNPDIKPKEHFKHLDRKRNEKFRYSFADNEGNITISGLLFFVSLFLEKKDAIWVQKKLEGFRCSNESYQKMTNEVFCRNRILMPKLRLESTQTQDWILLDMLNELIRCPKSLYERLKGEDRKKFEVPFDSTDEDYDAEQDPFKNTLIRYQDRFPYFVLRYFDYNEIFENLRFQIDLGTYHFSIYKKLIGGQKEDRHLTHKLYGFERLQEFAKQNRPDEWKAIIKDLDTFKKQEDEKVNEKPYISETTPHYHLENEKIGIAFKNHNIWPTTNTEFTNNKRKKYNLGTSIKAEAFLSVHELLPMMFYYMLLKIKNEGPRKVEAIIESKIKDIFTLYDAFANDEIDNTDELEKYCEDKDIKIGHLPKQMIAILYNKHKDMVEKAKVKQEKMKLATENRLKTLDKLIEGKMKYAKRGISPLKSGEIASWLVNDMMRFQPVQRDENGEPLNNSKANSTEYQLLQRTLALFGSEHKRLTPYFKQMNLIGDNNSHPFLKQTEWEECNNILSFYRSYLEARKNFLKNLEPKDWKKNQYFLKIKEPKSNRQTLVQGWKNGFNLPRGIFTEPIKKWFIEHRKKMKVDELKSVGLVAKVIPLYFNEKYKDSVQPFYNYPFNVGDINKPKEKDFLDNGKRRKLWDKKQLEFKGKTEEEKRKNPSYLDFKSWKKFERELRLIRNQDIVTWLMCIELYNNQKVENLNTGHIYLKDIITNVKEERNNNILNQIKSMQLPVTVYENESSPKKKYLRTIYIEENGTKLLKQGNFKALVKDRRLNGLFSFVKTSSEAESKRNPISKLRVEHELREYQKARIDIIKDMLALEKTLIDNYDSLDTDNFHNMLTGWLELKGEAIKTKFNNDVKLLTAVRNAFSHNQYPMYDENLFINIERFSLSSSRILESKGLGIAVKLKEEVSNAIEKIKNEEDAKKET

SEQ ID NO: 202 - protein, 1158 aa, synthetic construct
MKNQNSLPSNPTDILEDKPFWAAFFNLARHNVYLTVNHINKLIDLEKLYDEDKHEEIFEREDILNISDDVMNDVNSNGKKRKLDIKKIWDDLDTDLTRKYLLRELILKHFPFIQPAIIGAQTKERTTIDKDKRNTSTSNDSLKQTGEGDINDPLSLSNVKSMFFSLLQMLEQLRNYYSHVKHSKSATMPNFDEDLLNWMRYIFIDSVNKVKEDYSSNSVIDPNTCFSHLIYKDKQGKIKPCRYPFTSKDGSINAFGLLFFVSLFLEKQDSIWMQKKIPGFKKASENYMKMTNEVFCRNHILLPKIRLETVYDKDWMLLDMLNEVVRCPLSLYKRLTPADQNKFKVPEKSSDNANRQEDDNPFSRILVRHQNRFPYFVLRFFDLNEVFTTLRFQINLGCYHFAICKKQIGDKKEVHHLTRTLYGFSRLQNFTQNTRPEEWNTLVKTTEPSSGNDGKTVQGVPLPYISYTIPHYQIENEKIGIKIFDGDTAVDTDIWPSVSTEKQLNKPDKYTLTPGFKADVFLSVHELLPMMFYYQLLLCEGMLKTDAGNAVEKVLIDTRNAIFNLYDAFVQEKINTITDLENYLQDKPILIGHLPKQMIDLLKGHQRDMLKAAEQKKAMLIKDTERRLERLNKQPEQKPNVAAKNIGALLRNGQIADWLVKDMMRFQPVKRDKEGNPINCSKANSTEYQMLQRAFAFYATDSCRLPRYFEQLHLINCDNSHLFLSRFEYDKQPNLIAFYAAYLKAKLDFLNELQPQNWVSDNYFLLLRAPKNNRQKLAEGWKNGFNLPRGLFTEKIKTWFNEHKTIVDISDCDIFKNRVGQVARLIPVFFDKKFKDHSQPFYRYNFNVGNVSKPTEAKYLSKEKREELFKSYQNKFKNNIPAEKTKEYREYKNFSLWKKFERELRLIKNQDILTWLMCKNLFDEKIEQGIDIPYIKLDSLQTNTSTKGSLNALAQVLPMVLAIYIGNSESNNGTGANEEENKGPMVYIKEEGTKLLKWGNFKTLLADRRIKGLFSYIEHDDIDLKQHPLTKRRVDLELDLYQTCRIDIFQQTLGLEAQLLNKYSDLNTDNFYQMLIGWRKKEGIPRDIKEDTDFLKDVRNAFSHNQYPDSKKIAFSRIRKFNPKKTILNEKKGLGIAKQMYEEVEKVVNRIKGIELFD

SEQ ID NO: 203 - protein, 1165 aa, synthetic construct
MKNQNTLPSNPTDILKDKPFWAAFFNLARHNVYLTVNHINKLLDLEKLYNKDKHKEIFEHEDIFNISDDVMNDVNSNGKKRKLDIKKIWANLDTDLTRKYQLRELILKHFPFIQPAIIGAQTKERTTIDKDKRSTSTSNDSLKPTGEGDINDPLSLSNVKSIFFRLLQMLEQLRNYYSHVKHSKSATMPNFDEGLLKSMYNIFIDSVNKVKEDYSSNSVIDPNTSFSHLISKDEQGEIKPCRYSFTSKDGSINASGLLFFVSLFLEKQDSIWMQKKIPGFKKTSENYMKMTNEVFCRNHILLPKMRLETVYDKDWMLLDMLNEVVRCPLSLYKRLAPADQNKFKVPEKSSDNANRQEDDNPFSRILVRHQNRFPYFALRFFDLNEVFTTLRFQINLGCYHFAICKKQIGDKKEVHHLTRTLYGFSRLQNFTQNTRPEEWNTLVKTTEPSSGNDGKTVQGVPLPYISYTIPHYQIENEKIGIKIFDGDTAVDTDIWPSVSTEKQLNKPDKYTLTPGFKADVFLSVHELLPMMFYYQLLLCEGMLKTDAGNAVEKVLIDTRNAIFNLYDAFVQEKINTITDLENYLQDKPILIGHLPKQMIDLLKGHQRDMLKAVEQKKAMLIKDTERRLERLNKQPEQKPNVAAKNTGTLLRNGQIADWLVKDMMRFQPVKRDKEGNPINCSKANSTEYQMLQRAFAFYTTDSYRLPRYFEQLHLINCDNSHLFLSRFEYDKQPNLIAFYAAYLEAKLEFLNELQPQNWASDNYFLLLRAPKNDRQKLAEGWKNGFNLPRGLFTEKIKTWFNEHKTIVDISDCDIFKNRVGQVARLIPVFFDKKFKDHSQPFYTYNFNVGNVSKITEANYLSKEKRENLFKSYQNKFKNNIPAEKTKEYREYKNFSSWKKFERELRLIKNQDILTWLMCKNLFDEKIKPKKDILEPRIAVSYIKLDSLQTNTSTAGSLNALAKVVPMTLAIHIDSPKPKGKAGNNEKENKEFTVYIKEEGTKLLKWGNFKTLLADRRIKGLFSYIEHDDINLEKYPLTKYQVDSELDLYQKYRIDIFKQTLDLEAQLLDKYSDLNTDNFNQMLSGWSEKEGIPRNIKQDVAFLIGVRNGFSHNQYPDSKRIAFSRIKKFNPKTSSLQESEGLNIAKQMYEEAQQVVNKIKNIESFD

SEQ ID NO: 204 - protein, 809 aa, synthetic construct
MIKNPSNRHSLPKVIISEVDHEKILEFKIKYEKLARLDRFEVKAMHYEGKEIVFDEVLVNGGLIEVEYQDDNKTLFVKVGEKSYSIRGKKVGGKQRLLEDRVSKTKVQLELSDGVVDNKGNLRKSRTERELIVADNIKLYSQIVGREVTTTKEIYLVKRFLAYRSDLLFYYSFVDNFFKVAGNEKELWKINFDDATSAQFMGYIPFMVNDNLKNDNAYLKDYVRNDVQIKDDLKKVQTIFSALRHTLLHFNYEFFEKLFNGEDVGFDFDIGFLNLLIENIDKLNIDAKKEFIDNEKIRLFGENLSLAKVYRLYSDICVNRVGFNKFINSMLIKDGVENQVLKAEFNRKFGGNAYTIDIHSNQEYKRIYNEHKKLVIKVSTLKDGQAIRRGNKKISELKEQMKSMTKKNSLARLECKMRLAFGFLYGEYNNYKAFKNNFDTNIKNSQFDVNDVEKSKAYFLSTYERRKPRTREKLEKVAKDIESLELKTVIANDTLLKFILLMFVFMPQELKGDFLGFVKKYYHDVHSIDDDTKEQEEDVVEAMSTSLKLKILGRNIRSLTLFKYALSSQVNYNSTDNIFYVEGNRYGKIYKKLGISHNQEEFDKTLVVPLLRYYSSLFKLMNDFEIYSLAKANPTAVSLQELVDDETSPYKQGNYFNFNKMLRDIYGLTSDEIKSGQVVFMRNKIAHFDTEVLLSKPLLGQTKMNLQRKDIVSFIEARGDIKELLGYDAINDFRMKVIHLRTKMRVYSDKLQTMMDLLRNAKTPNDFYNVYKVKGVESINKHLLEVLAQTAEERTVEKQIRDGNEKYDL

SEQ ID NO: 205 - protein, 840 aa, synthetic construct; identical to SEQ ID NO: 11

SEQ ID NO: 206 - protein, 1238 aa, synthetic construct
MNQYIHANKKENKKRPNKSSIIRIMVSDFDDEYIQEIKVLYIKQGGVDTFKINKMSYDSASKKIIFEEVATQNMLSVEDSNLNFKRPMVECKNGDIYVVKPSEKVNEKGQAIEPLRSYKIHGKYLDLTEEIGKDNAEQSGKKQIYLHVEDLLGMHTTADSIDRRRLESETQRTLLSKEVMENYALIMGHEIKLDESDETYKLASSKEIYKANRFLDYRSRLLYYYSFINHFLVGLSKGATYEYAGKSLVAKIPDGEVWQLCELEVTAYHGIYINKKRNELVNENSLKIDAIYAQMAKNMKETINEFVDNYNAMIEKQNELKDNKSYKINKKAQNTTKFENFTEEKINQDLHNIVYILSDLRHKLMHFEYHYFELLMTGKKMGEKSEVIVCVPDRNSELPASKNEPSKDKFRKEKKLSELLDLNILKELDTFVKVKESYQTTYLETNDKIEILGKLKTAKSIYQIYHQICQRKNGFNKFINSFFTVDGEENTEVKDCINEVFRKEIHYFEMVIAKSNEDTLNDKNKNKSKKTRDRMKNQIQECKRYQDDESNIDTWVAYHKDIHYSKRYKKLYCEHITLVDNLNSAVSNGLNGQVIKEINDDIAIKKREMNEITKANSKSRLRYKMQMAYGFLFVEYGLKIPKFLNDFDLSHVRTASKIKGYKSPVKVTQYLTNDEGNKDNFNLETLMEDIDKKSKINFEFLKSNEDNNLIKLYILIYQLLPRELKGDFLGFVKNNYYDLKHVDFQTRETEAKDQFFHNMRLFEKNVKAFDLIQYSIGDEMSQLGNETFNFSQALSKIVSDDTILNSATEIPNINRLVYGSLLKYYENAFRLSSEIEIRALIKIARGKQIDNHSIDEAYKDALIFQKSGQTVKFSSILKYFNIDDLNKKNDSKIYNKASNLRNKIAHCDYTVLFITNIICEAENINVKAKYLIDVSNKIGLNTVDLGNDMVNDYLMSYDKQMTYLAKTSEELLKESSLDKSKEKKERRDNLKNETRSHSEIYMEYEWISDYLVKLKDNHEILSKKNKTEDLKLNRAYELIKNYNVALNKKSKIKIRALTTDHINNNWVNIWGALTHELQEIKGLYLYKVTPKIKKGIYFVLTDKKNYCLNINLYTLKKVPSKDDSEMTEERYLRDISEKYYFTVGEDSIKALKERLQTINDKCIITYVNEIDCKNENIKEKPEFKIAEDYNSWDKKYLSISNEELHHWSVETRRDKKYKENLILNLMEKSSFKLNLHQL

SEQ ID NO: 207 - protein, 849 aa, synthetic construct
MSQLKNPSNKNSLPRIIISDFNEIKINEIKIKYHKLDRLDKIIVKEMEIINNKIFFKKILFNNQIKDINSENIELENYILAGEVKPSNTKIILNRDGKEKSFIVYDGFTFKYKPNDKRISETKTNAKYILTIKDKTRHRESSTQRDILKSSIIETYKQISGFENITSKDIYTIKRYIDFKNEMMFYYTFIDDFFFPITGKNKQDKKNNFYNYKIKENAKKFISLINYRINDDFKNKNGILYDYLSNKEEIIINDFIHIQTILKDVRHAIAHFNFDFIQKLFDNEQAFNSKFDGIEILNILFNQKQEKYFEAQTNYIEEETIKILDEKELSFKKLHSFYSQICQKKPAFNKLINSFIIQDGIENKELKDYISQKYNSKFDYYLDIHTCKIYKDIYNQHKKFVADKQFLENQKTDGQKIKKLNDQINQLKTKMNNLTKKNSLKRLEIKFRLAFGFIFTEYQTFKNFNERFIEDIKANKYSTKIELLDYGKIKEYISITHEEKRFFNYKTFNKKTNKNINKTIFQSLEKETFENLVKNDNLIKMMFLFQLLLPRELKGEFLGFILKIYHDLKNIDNDTKPDEKSLSELNISTALKLKILVKNIRQINLFNYTISNNTKYEEKEKRFYEEGNQWKDIYKKLYISHDFDIFDIHLIIPIIKYNINLYKLIGDFEVYLLLKYLERNTNYKTLDKLIEAEELKYKGYYNFTTLLSKAINIALNDKEYHNITHLRNNTSHQDIQNIISSFKNNKLLEQRENIIELISKESLKKKLHFDPINDFTMKTLQLLKSLEVHSDKSEKIENLLKKEPLLPNDVYLLYKLKGIEFIKKELISNIGITKYEEKIQEKIAKGVEK

SEQ ID NO: 208 - protein, 779 aa, synthetic construct
ELCKIDFTDARSSSLIEYFKFAINDNLKNDRGYLKAYVNDVEQIRADLKKVGGKQRNLEDRVSRTKVQLTLTNHIEDREGKQRVSRTERELIVPQNIKLYSQIVGREVKTTKEIYLIKRFLEYRSDLLFYYGFVDNFFKVEGNKKELCKIDFTDARSSSLIEYFKFAINDNLKNDRGYLKAYVNDVEQIRTDLQKVKTIFSKLRHALMHFDYDFFEKLFNGEEVGFDFDIKFLNIMIDKVEKLNIETKKEFIEDEVITLFGERLSLKKLYGLFSHIAINRVAFNKFINSFLIKDGIENRALKDFFNDEKGSQAYEIDIHSNAEYKALYVQHKKLVMATSAMSDGNEIAKKNQEISELKEKMNAITKANSLARLEYKLRLAFGFIYTEYGDYTAFKNSFDRDVKSAKYKELSVERLKAYYLATFKASKPQSHEKLEEVAKKIDRLSLKQLIENETLLKFVLLLFTFMPQELKGEFLGFIKKYYHDKKHIEQDTKEKEEEREGLSTGLKLKVLEKNIRSLSILKHALSFQVKYNKKDKNFYEEGNLHGKFYKKLAISHNQEEFNKSVYAPLFRYYVALYKLINDFEIYSLAQHIVNNETLADQVGKAQFRQRGYFNFRKLVNCTYATAQNSSYNVLIFMRNDISHLSYEPLFNCPLEEKASYKQKIRGREKIISVKPLSESRAEIVRFIASQTDMKKLLGYDAVNDFNMKMVQLRRRLSVYANKQETIEKMINKAKTPNDFYNLYKLKGIECINQHLLKVIGVTEAEKRIEKQIEEGNEKY

SEQ ID NO: 209 - protein, 802 aa, synthetic construct
MLKKPSNRYALPKVILSTVDHEKILEFKVKYEKLARLDRLVVERMHFDGESVVFDEVIANSGDLEIAYQDDHRKLLIQAAGKSYTITGKKVGGKKRKLEERISRAKIQLTLTDGQEDQHRRIRATVTEKALLEPKEDRDIYSKISDRKIKTSKEIYLVKRFLSYRSDLLFYYFFVDNFFKVGNNKQELWKIKFQNQPELIEYFRFIINDRFKNAKNDKFDNYLKNDKAIQEDLEKIQKVFEKLRHALMHYDYGFFEKLFGGEDQGFDLDIAFLDNFVKKIDKLNIDTKKEFVDDEKIKIFGEDLNLADLYKLYASISINRVGFNRVVNEMIIKDGIEKSELKRAFEKKLDKTYALDIHSDPSYKKLYNEHKRLVTEVSTYTDGNKIKEGNQKIAKLKYEMKEITKKNALVRLECKMRLAFGLIYGRYDTHEAFKNGFDTDLKRGEFAQIGSEEAIGYFNTTFEKSKPKSKEEIKKIARQIDNLSLSTLIEDDPLMKFIVLMFLFVPRELKGEFLGFWRKYYHDIHSIDSDAKSDEMPDEVSLSLKLKILTRNIRRLNLFEYSLSEKIKYSPKNTQFYTDKSPYQKVYKRLKISHNKEEFDKTLLVPLFRYYSILFKLINDFEIYSLAKANPDASSLSELTKTKHGFRGHYNFTTLMMDAHKVSQGDSKKHFGIRGEIAHINTKDLIYDPLFRKSKMAQQRNDVIDFVLKYEKEIKAVLGYDAINDFRMKVVQLRTKLKVYSDKTQTIEKLLNEVEAPDDFYVLYKVKGVEAINKYLLEIVSVTQAEEEIERKIITGNKRYNT

SEQ ID NO: 210 - protein, 840 aa, synthetic construct
MLKHKRKNKNSLARVVLSNYDSNNIYEIKIKYEKLAKLDKINIIEMDYDADNNVMFKKVLFNNKEIDLSHKDKTKINIELDNKKYNISAKKQIGKTHLVVRNKQTSKISRIKKIQDTYYRGKDVFILDNNIEILDKKQTKDKFIVTLNDITNNKTTSTEAELIDDTKDIFKKISAKKDLKSSDIYKIKRFISIRSNFSFYYTFVDNYFKIFHAKKDKNKEELYKIKFKDEINIKPYLENILDNMKNKNGILYNYANDRKKVLNDLRNIQYVFKEFRHKLAHFDYNFLDNFFSNSVEEKYKQKVNEIKLLDILLDNIDSLNVVPKQNYIEDETISVFDAKDIKLKRLYTYYIKLTINYPGFKKLINSFFIQDGIENQELKEYINNKEKDTQVLKELDNKAYYMDISQYRKYKNIYNKHKELVSEKELSSDGKKINSLNQKINKLKIDMKNITKPNALNRLIYRLRVAFGFIYKEYATINNFNKSFLQDTKTKRFENISQQDIKSYLDISYQDKGKFFVKSKKTFKNKTTVKYTFEDLDLTLNEIITQDDIFVKVIFLFSIFMPKELNGDFFGFINMYYHKMKNISYDTKDIDMLDTISQNMKLKILEQNIKKTYVFKYYLDLDSSIYSKLVQNIKITEDIDSKKYLYAKIFKYYQHLYKLISDVEIYLLYKYNSKENLSITIDKDELKHRGYYNFQSLLIKNNINKDDAYWSIVNMRNNLSHQNIDELVGHFCKGCLRKSTTDIAELWLRKDILTITNEIINKIESFKDIKITLGYDCVNDFTQKVKQYKQKLKASNERLAKKIEEKQNQVVDEKNKEELEKNILNMKNIQKINRYILDIL

SEQ ID NO: 211 - protein, 845 aa, synthetic construct
MIKKPSNRHALPKVIISKVDNQNILEFKIKYKKLSRLDRVEIKTMHYDDRAIVFDEVIINGGLIDVEYRDNHKTIFVKVGDKSYSISGQKVGGKERLLENRISQTKVQLELKDEATNRVSKTERELIVDDNIKLYSQIVGRDVKTTKDIYLIKRFLGYRSDLLFYYGFVNNFFHVANNRPEFWKIDFNDNRNSKLIEYFIFTINDHLKNDENYLKDYISDRGQIVDDLENIKHIFSALRHGLMHFDYDFFEALFNGEDIDIKMDNQGNTQPLSSLNIKFLDIMIDKLDKLNIDTKKEFIDAEKITIFGEELSLAKLYRFYAHTAINRVAFNKLINSFIIENGVENQSLKEYFNQQAGGIAYEIDIHQNREYKNLYNEHKKLVSRVLSISDGQEIATLNQKIVELKEQMKQITKINSIKRLEYKLRLAFGFIYTEYKNYEEFKNSFDTDIKNGRFTPKDEDGNKRAFDSRELEHLKGYYKATLQTQKPQTDEKMEEVSKRVDRLSLKSLIGDDTLLKFILLMFTFMPQELKGEFLGFIKKYYHDTKHIDQDTISDSDDTIEEGLSIGLKLKILDKNIRSLSILKHSLSFQTKYNKKDRSYYEDGNIHGKFFKKLGISHNQEEFNKSVYAPLFRYYSALYKLINDFEIYTLSLHIVGNETLSDQVNKPQFLSGRYFNFRKLLTQSYNISNNSTHSVIFNAVINMRNDISHLSYEPLLDCPLNGKKSYKRKIRNQFRTINIKPLVESRKMIIDFITLQTDMQKVLGCDAVNDFTMKIVQLRTRLKAYANKEQTIEKMITEAKTPNDFYNIYKVKGVEAINKYLLEVIGETQVEKEIREEIERGNIANS

SEQ ID NO: 212 - protein, 822 aa, synthetic construct
MVKNPANRHALPKVIISEVDNNNILEFKIKYEKLARLDKVEVKSMHFDNNKQVVFDEVVINGGLIEPTYEDKHKKLVVTAGEKSYSIVGQKVGGKPRLLEDRVSKTKVQLELTNYVEDKEGKKRVSKTERELIVADNIELYSQIVGREVKTTKEIYLIKRFLEYRSDLLFYYGFVDNFFKVAGNGKELWKIDFTNSDSLHLIEYFKFSINDNLKNDENYLKNYVSDNTKIENDLVKCQNNFNSLRHALMHFDYDFFEKLFNGEDVGFDFDIEFLNIMIDKVDKLNIDTKKEFIDDEEVTLFGEALSLKKLYGLFSHIAINRVAFNKLINSFIIEDGIENKELKDFFNNKKESQAYEIDIHSNAEYKALYVQHKKLVMATSAMTDGDEIAKKNQEISDLKEKMKVITKENSLARLEHKLRLAFGFIYTEYKDYKTFKKHFDQDIKGAKYKGLNVEKLKEYYETTLKNSKPKTDEKLEDVAKKIDKLSLKELIDDDTLLKFVLLLFIFMPQELKGDFLGFIKKYYHDKKHIDQDTKDKDTEIEELSTGLKLKVLDKNIRSLSILKHSFSFQVKYNRKDKNFYEDGNLHGKFYKKLSISHNQEEFNKSVYAPLFRYYSALYKLINDFEIYALAQHVENHETLADQVNKSQFIQKSYFNFRKLLDNTDSISQSSSYNTLIVMRNDISHLSYEPLFNYPLDERKSYKKKTQKGVKTFHVELLYISRAKIIELISLQTDMKKLLGYDAVNDFNMKVVHLRKRLSVYANKEESIRKMQADAKTPNDFYNIYKVKGVESINQHLLKVIGVTEAEKSIEKQINEGNKKHNT

SEQ ID NO: 213 - protein, 832 aa, synthetic construct
MIKNPSNRYALPKVIISKIDNQNILEFKIKYKKLSKLDIVKVKSMHYDDRAIIFDEVIVNDGLIDVEYRDNHKTIFVKVGNKSYSISGQKVGGKERLLENRVSKTKVQLELKDKATNRVSKTERELIVDDNIKIYSQIVGRDVKTTKDIYLIKRFLAYRSDLLFYYGFVNNFFHVANNRSEFWKIDFNDSNNSKLIEYFKFTINDHLKNDENYLKDYISDNEKLKNDLIKVKNSFEKIRHALMHFDYDFFVKLFNGEDVGLELDIEFLDIMIDKLDKLNIDTKKEFIDDEKITIFGEELSLAKLYRFYAHTAINRVAFNKLINSFIIENGVENQSLKEYFNQQAGGIAYEIDIHQNREYKNLYNEHKKLVSRVLSISDGQEIAILNQKIAKLKDQMKQITKANSIKRLEYKLRLALGFIYTEYENYEEFKNNFDTDIKNGRFTPKDNDGNKRAFDSRELEQLKGYYEATIQTQKPKTDEKIEEVSKKIDRLSLKSLIADDILLKFILLMFTFMPQELKGEFLGFIKKYYHDTKHIDQDTISDSDDTIETLSIGLKLKILDKNIRSLSILKHSLSFQTKYNKKDRNYYEDGNIHGKFFKKLGISHNQEEFNKSVYAPLFRYYSALYKLINDFEIYTLSLHIVGSETLTDQVNKSQFLSGRYFNFRKLLTQSYHINNNSTHSTIFNAVINMRNDISHLSYEPLFDCPLNGKKSYKRKIRNQFKTINIKPLVESRKIIIDFITLQTDMQKVLGYDAVNDFTMKIVQLRTRLKAYANKEQTIQKMITEAKTPNDFYNIYKVQGVEEINKYLLEVIGETQAEKEIREKIERGNIANF

SEQ ID NO: 214 - protein, 806 aa, synthetic construct
MTKKPSNRNSLPKVIINKVDESSILEFKIKYEKLARLDRFEVRSMRYDGDGRIIFDEVVANAGLLDVDYEDDNRTIVVKIENKAYNIYGKKVGGEKRLNGKISKAKVQLILTDSIRKNANDTHRHSLTERELINKNEVDLYSKIAEREISTTKDIYLVKRFLAYRSDLLLYYAFINHYVRVNGNKKEFWKTEIDDKIIDYFIYTINDTLKNKEGYLEKYIVDRDQIKKDLEKIKQIFSHLRHKLMHYDFRFFTDLFDGKDVDIKVDNSIQKISELLDIEFLNIVIDKLEKLNIDAKKEFIDDEKITLFGQEIELKKLYSLYAHTSINRVAFNKLINSFLIKDGVENKELKEYFNAHNQGKESYYIDIHQNQEYKKLYIEHKNLVAKLSATTDGKEIAKINRELADKKEQMKQITKANSLKRLEYKLRLAFGFIYTEYKDYERFKNSFDTDTKKKKFDAIDNAKIIEYFEATNKAKKIEKLEEILKGIDKLSLKTLIQDDILLKFLLLFFTFLPQEIKGEFLGFIKKYYHDITSLDEDTKDKDDEITELPRSLKLKIFSKNIRKLSILKHSLSYQIKYNKKESSYYEAGNVFNKMFKKQAISHNLEEFGKSIYLPMLKYYSALYKLINDFEIYALYKDMDTSETLSQQVDKQEYKRNEYFNFETLLRKKFGNDIEKVLVTYRNKIAHLDFNFLYDKPINKFISLYKSREKIVNYIKNHDIQAVLKYDAVNDFVMKVIQLRTKLKVYADKEQTIESMIQNTQNPNGFYNIYKVKAVENINRHLLKVIGYTESEKAVEEKIRAGNTSKS

SEQ ID NO: 215 - protein, 835 aa, synthetic construct
MEKIKKPSNRNSIPSIIISDYDANKIKEIKVKYLKLARLDKITIQDMEIVDNIVEFKKILLNGVEHTIIDNQKIEFDNYEITGCIKPSNKRRDGRISQAKYVVTITDKYLRENEKEKRFKSTERELPNNTLLSRYKQISGFDTLTSKDIYKIKRYIDFKNEMLFYFQFIEEFFNPLLPKGKNFYDLNIEQNKDKVAKFIVYRLNDDFKNKSLNSYITDTCMIINDFKKIQKILSDFRHALAHFDFDFIQKFFDDQLDKNKFDINTISLIETLLDQKEEKNYQEKNNYIDDNDILTIFDEKGSKFSKLHNFYTKISQKKPAFNKLINSFLSQDGVPNEEFKSYLVTKKLDFFEDIHSNKEYKKIYIQHKNLVIKKQKEESQEKPDGQKLKNYNDELQKLKDEMNTITKQNSLNRLEVKLRLAFGFIANEYNYNFKNFNDEFTNDVKNEQKIKAFKNSSNEKLKEYFESTFIEKRFFHFSVNFFNKKTKKEETKQKNIFNSIENETLEELVKESPLLQIITLLYLFIPRELQGEFVGFILKIYHHTKNITSDTKEDEISIEDAQNSFSLKFKILAKNLRGLQLFHYSLSHNTLYNNKQCFFYEKGNRWQSVYKSFQISHNQDEFDIHLVIPVIKYYINLNKLMGDFEIYALLKYADKNSITVKLSDITSRDDLKYNGHYNFATLLFKTFGIDTNYKQNKVSIQNIKKTRNNLAHQNIENMLKAFENSEIFAQREEIVNYLQTEHRMQEVLHYNPINDFTMKTVQYLKSLSVHSQKEGKIADIHKKESLVPNDYYLIYKLKAIELLKQKVIEVIGESEDEKKIKNAIAKEEQIKKGNN

SEQ ID NO: 216 - protein, 847 aa, synthetic construct
MMTKKPANRHALPKVIISEVDNTNILEFKIKYEKLARLDRVEVKAMHYEDGRIIFDEVVVNGGLIEVEYQDDHKTLFVQVGEKSYSISGQKVGGKQRLLEDRVSKTKVQLELSDGSSERVSRTERELIVADNIKLYSQIVGHEVKTTKEIYLAKRFLGYRSDLLFYYGFVDNFFRESKNLKYGKQPVELWEDKFQVNDKLTAYTKFMFNDDLQNSESYLKEYVKDNHKIKNDLESARDIFATFRHNLMHFNYSFFTRLFNGEDVKIKNLQTKKFESLSDVLRNVEFLNKVIQSIDKLNIDTRKEFIDKEKITLFNEELDLQQLYGFFAYTAINRVAFNKLINSFIIKDGIENEQLKEYFNQRVDGTAYEIDIHQNREYKELYKKHKNLVSKVSTLSDGKEIARGNTEISVLKEQMNKITKANSLKRLEHKLRLAFGFIYTEYGSYKAFVSRFNEDTKRKKIKNVEFEKIGVEKQKEYYESTFTSNNKDKLGELIQEYEKLSLNDLIENDTFLKVILLLFIFMPKEVKGDFLGFIKKYYHDTKHIEEDTKEKDEGFTNTLPIGLKLKIVERNIAKLSVLKHSLSLKVKYNRGQYEEDNTYRKVFKKLNISHNQEEFHKSMFSPLLRYYASLYKLINDFEIYTLSHYITDKYSTLNKVIASEQFHYRYGWNREEKKGELVKTDNYTFSTLLSKKYGHKNSQEISEMRNKISHFDEKILFKFPLEEVSSVPKGKGKYKKDEPIKSLKEKREEIVSLMEKQTDMQKVLGYDAINDFRMKTVQFQTKLKVYSNKEETIKKMIVEAKTPNDYYNIYKVKGVEGINEHLLNVIGETEAEKSIQEQIAEGNKVNV

SEQ ID NO: 217 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVNVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 218 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIANLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 219 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIQNLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAV

SEQ ID NO: 220 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNAAIDVATENLATVAITAQSMPKPTCERSGDAKKPVRARKAKAPEFHDKLAPSYTVVLREAV

SEQ ID NO: 221 - protein, 717 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNANIDVATENLATVAITAQSMPKPTCERSGDAKKPVRARKAKAPEFHDKLAPSYTVVLREAV

SEQ ID NO: 222 - protein, 469 aa, synthetic construct
MPTITRKIELTLCTEGLSDQERKDQWNLLYHINDNLYRAANNISSKLYLDDHVGSMVRLKHAEYLSLLRALEKAKKQKAPDEEVIAELSQQVATAEQEMDEQAKAICQYATEMSTQTLSYRFATELETNIFGQILTCLRQGVFSTFNSDARDVKRGERSIRTYKKGMPIPFPWNDSLRIGFEDGEFYLRWYNGLRFRFDFGKDRSNNCLIVQRCMKMDKDYEGDYKLCNSSIQMVKREGKPKFFLLLVVNIPQERVELNKNIVVGVALGINAPAYVATNTTPERKQIGDREHFLNERMAFQRRFKSLQRLKGTTGGRGRAKKLEPLERLRKAEQNWVHTQNHLFSREVIDFAVKARAATIHMEDLSGFGKDNDGNADERKEFVLRNWSYYELQNMITYKAAKYGIKVEKIRPAYTSKTCSWCGHQGFREGITFICENPECKKFGEKEHADYNAARNIANSKEIIKNNEE

SEQ ID NO: 223 - protein, 468 aa, synthetic construct; identical to SEQ ID NO: 177

SEQ ID NO: 224 - protein, 468 aa, synthetic construct; identical to SEQ ID NO: 178

SEQ ID NO: 225 - protein, 468 aa, synthetic construct; identical to SEQ ID NO: 179

SEQ ID NO: 226 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIAVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 227-228 - skipped (2 entries)

SEQ ID NO: 229 - protein, 8 aa, synthetic construct
  VARIANT 3: note = X is any naturally occurring amino acid
  VARIANT 5: note = X is any naturally occurring amino acid
  VARIANT 6: note = X is any naturally occurring amino acid
  VARIANT 4: note = X is Trp or Phe or Tyr
KRXXXXAF

SEQ ID NO: 230 - skipped

SEQ ID NO: 231 - protein, 6 aa, synthetic construct
  VARIANT 5: note = X is Lys or Arg
  VARIANT 6: note = X is Trp or Phe or Tyr
LGKRXX

SEQ ID NO: 232-238 - skipped (7 entries)

SEQ ID NO: 239 - protein, 16 aa, synthetic construct
APKKKRKVGIHGVPAA

SEQ ID NO: 240 - protein, 20 aa, synthetic construct
  VARIANT 4: note = X is any naturally occurring amino acid
  VARIANT 8: note = X is any naturally occurring amino acid
  VARIANT 11: note = X is any naturally occurring amino acid
  VARIANT 15: note = X is any naturally occurring amino acid
  VARIANT 19: note = X is any naturally occurring amino acid
GLFXALLXLLXSLWXLLLXA

SEQ ID NO: 241-299 - skipped (59 entries)

SEQ ID NO: 300 - RNA, 78 nt, synthetic construct
  source 1..78: mol_type = other RNA
tggggcagttggttgcccttagcctgaggcatttattgcactcgggaagtaccatttctcagaaatggtacatccaac

SEQ ID NO: 301 - RNA, 79 nt, synthetic construct
  source 1..79: mol_type = other RNA
atggggcagttggttgcccttagcctgaggaatttaattcactcgggaagtacctttctcatgaaatggtacatccaac

SEQ ID NO: 302 - RNA, 141 nt, synthetic construct
  source 1..141: mol_type = other RNA
accgcttcaccaagtgctgtcccttaggggattagcacttgagtgaaggtgggctgcttgcatcagcctaatgtcgagaagtgctttcttcggaaagtaaccctcgaaacaaattcatttgaaagaatgaaggaatgcaac

SEQ ID NO: 303-399 - skipped (97 entries)

SEQ ID NO: 400 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 401 - protein, 166 aa, synthetic construct
SEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 402 - protein, 198 aa, synthetic construct
EASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 403 - protein, 228 aa, synthetic construct
SSETGPVAVDPTLRRRIEPHEFEVFFDPRELRKETCLLYEIKWGTSHKIWRHSSKNTTKHVEVNFIEKFTSERHFCPSTSCSITWFLSWSPCGECSKAITEFLSQHPNVTLVIYVARLYHHMDQQNRQGLRDLVNSGVTIQIMTAPEYDYCWRNFVNYPPGKEAHWPRYPPLWMKLYALELHAGILGLPPCLNILRRKQPQLTFFTIALQSCHYQRLPPHILWATGLK

SEQ ID NO: 404 - protein, 224 aa, synthetic construct
MAQKEEAAAAAEPASQNGEEVENLEDPEKLKELIELPPFEIVTGERLPAHYFKFQFRNVEYSSGRNKTFLCYVVEAQSKGGQVQASRGYLEDEHATNHAEEAFFNSIMPTFDPALRYMVTWYVSSSPCAACADRIVKTLNKTKNLRLLILVGRLFMWEEPEIQAALRKLKEAGCRLRIMKPQDFEYIWQNFVEQEEGESKAFEPWEDIQENFLYYEEKLADILK

SEQ ID NO: 405 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 406 - protein, 854 aa, synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGGGSGGGGSGGGGSPKKKRKVEASGGGSGGGSGGGSGEAAPCDCDGGTQKEKGPYYTHLGAGPSVAAVRELMETRFGQKGKAIRIEKIVFTGKEGKSSQGCPVAKWVIRRSGPEEKLICLVRERVDHHCSTAVIVVLILLWEGIPRLMADRLYKELTENLRSYSGHPTDRRCTLNKKRTCTCQGIDPKTCGASFSFGCSWSMYFNGCKFGRSENPRKFRLAPNYPLHNYYKRITGMSSEGSDVKTGWIIPDRKTLISREEKQLEKNLQELATVLAPLYKQMAPVAYQNQVEYEEVAGDCRLGNEEGRPFSGVTCCMDFCAHSHKDIHNMHNGSTVVCTLIRADGRDTNCPEDEQLHVLPLYRLADTDEFGSVEGMKAKIKSGAIQVNGPTRKRRLRFTEPVPRCGKRAKMKQNHNKSGSHNTKSFSSASSTSHLVKDESTDFCPLQASSAETSTCTYSKTASGGFAETSSILHCTMPSGAHSGANAAAGECTGTVQPAEVAAHPHQSLPTADSPVHAEPLTSPSEQLTSNQSNQQLPLLSNSQKLASCQVEDERHPEADEPQHPEDDNLPQLDEFWSDSEEIYADPSFGGVAIAPIHGSVLIECARKELHATTSLRSPKRGVPFRVSLVFYQHKSLNKPNHGFDINKIKCKCKKVTKKKPADRECPDVSPEANLSHQIPSRVASTLTRDNVVTVSPYSLTHVAGPYNRWVAAADYKDDDDK

SEQ ID NO: 407 - protein, 1006 aa, synthetic construct
QSQPGHNQMLRPIKTEPVSKPSSYRYPLSPPQENMSSRIKQEISSPSRDNGQPKSIIETMEQHLKQFQLKSLCDYKALTLKSQKHVKVPTDIQAAESENHARAAEPQATKSTDCSVLDDVSESDTPGEQSQNGKCEGCNPDKDEAPYYTHLGAGPDVAAIRTLMEERYGEKGKAIRIEKVIYTGKEGKSSQGCPIAKWVYRRSSEEEKLLCLVRVRPNHTCETAVMVIAIMLWDGIPKLLASELYSELTDILGKCGICTNRRCSQNETKKKQSPPRNCCCQGENPETCGASFSFGCSWSMYYNGCKFARSKKPRKFRLHGAEPKEEERLGSHLQNLATVIAPIYKKLAPDAYNNQVEFEHQAPDCCLGLKEGRPFSGVTACLDFSAHSHRDQQNMPNGSTVVVTLNREDNREVGAKPEDEQFHVLPMYIIAPEDEFGSTEGQEKKIRMGSIEVLQSFRRRRVIRIGELPKSCKKKAEPKKAKTKKAARKHSSLENCSSRTEKGKSSSHTKLMENASHMKQMTAQPQLSGPVIRQPPTLQRHLQQGQRPQQPQPPQPQPQTTPQPQPQPQHIMPGNSQSVGSHCSGSTSVYTRQPTPHSPYPSSAHTSDIYGDTNHVNFYPTSSHASGSYLNPSNYMNPYLGLLNQNNQYAPFPYNGSVPVDNGSPFLGSYSPQAQSRDLHRYPNQDHLTNQNLPPIHTLHQQTFGDSPSKYLSYGNQNMQRDAFTTNSTLKPNVHHLATFSPYPTPKMDSHFMGAASRSPYSHPHTDYKTSEHHLPSHTVYSYTAAASGSSSSHAFHNKENDNIANGLSRVLPGFNHDRTASAQELLYSLTGSSQEKQPEVSGQDAAAVQEIEYWSDSEHNFQDPCIGGVAIAPTHGSILIECAKCEVHATTKVNDPDRNHPTRISLVLYRHKNLFLPKHCLALWEAKMAEKARKEEECGKNGSDHVSQKNHGKQEKREPTGPQEPSYLRFIQSLAENTGSVTTDSTVTTSPYAFTQVTGPYNTFV

SEQ ID NO: 408 - protein, 617 aa, synthetic construct
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 409 - protein, 523 aa, synthetic construct
DALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 410 - protein, 322 aa, synthetic construct
TYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 411 - protein, 380 aa, synthetic construct
MAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFST

SEQ ID NO: 412 - protein, 50 aa, synthetic construct
DALDDFDLDMLGSDALDDFDLDMLGSDALDDFDLDMLGSDALDDFDLDML

SEQ ID NO: 413 - protein, 745 aa, synthetic construct
GQTGKKSEKGPVCWRKRVKSEYMRLRQLKRFRRADEVKTMFSSNRQKILERTETLNQEWKQRRIQPVHIMTSVSSLRGTRECSVTSDLDFPAQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPDEREEKQKDLEDNRDDKETCPPRKFPADKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTISVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPVPTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 414 - protein, 75 aa, synthetic construct
MDAKSLTAWSRTLVTFKDVFVDFTREEWKLLDTAQQIVYRNVMLENYKNLVSLGYQLTKPDVILRLEKGEEPWLV

SEQ ID NO: 415 - protein, 99 aa, synthetic construct
NNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESL

SEQ ID NO: 416 - protein, 479 aa, synthetic construct
VGTMITSALHRAADWAKSVFSSAALGDPRRTARLVNVAAQLAKYSGKSITISSEGSKAMQEGAYRFIRNPNVSAEAIRKAGAMQTVKLAQEFPELLAIEDTTSLSYRHQVAEKLGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIAQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKI

SEQ ID NO: 417 - protein, 340 aa, synthetic construct
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 418 - protein, 509 aa, synthetic construct
MPIAPEFLSLAYPGQEFPAYLYGRASRDPKRKGRSVQSQLDEGRATCLDAGWPIAGEFKDVDRSASAYARRTRDEFEEMIAGIQAGECRILVAFEASRYYRDLEAYVRLRRVCREAGVLLCYNGQVYDLSKSADRKATAQDAVNAEGEADDIRERNLRTTRLNAKRGGAHGPVPDGYKRRYDPDSGDLVDQIPHPDRAGLITEIFRRAAAAEPLAAICRDLNERGETTHRGKAWQRHHLHAILRNPAYIGHRRHLGVDTGKGMWAPICDDEDFAETFQAVQEILSLPGRQLSPGPEAQHLQTGIALCGEHPDEPPLRSVTVRGRTNYNCSTRYDVAMREDRMDAFVEESVITWLASDEAVAAFEDNTDDERTRKARIRLKVLEEQLEAAQKQARTLRPDGMGMLLSIDSLAGLEAELTPQIDKARQESRSLHVPALLRDLLGKPRADVDRAWNEALTLPQRRMILRMVVTIRLFKAGSRGVRAIEPGRITLSYVGEPGFKPVGGNRAKQ

SEQ ID NO: 419 - protein, 474 aa, synthetic construct
MAVGIYIRVSTQEQASEGHSIESQKKKLASYCEIQGWDDYRFYIEEGISGKNTNRPKLKLLMEHIEKGKINILLVYRLDRLTRSVIDLHKLLNFLQEHGCAFKSATETYDTTTANGRMSMGIVSLLAQWETENMSERIKLNLEHKVLVEGERVGAIPYGFDLSDDEKLVKNEKSAILLDMVERVENGWSVNRIVNYLNLTNNDRNWSPNGVLRLLRNPALYGATRWNDKIAENTHEGIISKERFNRLQQILADRSIHHRRDVKGTYIFQGVLRCPVCDQTLSVNRFIKKRKDGTEYCGVLYRCQPCIKQNKYNLAIGEARFLKALNEYMSTVEFQTVEDEVIPKKSEREMLESQLQQIARKREKYQKAWASDLMSDDEFEKLMVETRETYDECKQKLESCEDPIKIDETYLKEIVYMFHQTFNDLESEKQKEFISKFIRTIRYTVKEQQPIRPDKSKTGKGKQKVIITEVEFYQ

SEQ ID NO: 420 - protein, 805 aa, synthetic construct
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSRSWDDKTCQKSANVSSIWHRNYSPRPESGTVSDAPQLKENPSTVGVERVISTKGLNLPRKSSIVKRPRSAELSEDDLLSQYSLSFTKKTKKNSSEGNKSLSFSEVFVPDLVNGPTNKKSVSTPPRTRNKFATFLQRKNEESGAVVVPGTRSRFFCSSDSTDCVSNKVSIQPLDETAVTDKENNLHESEYGDQEGKRLVDTDVARNSSDDIPNNHIPGDHIPDKATVFTDEESYSFKSSKFTRTISPPTLGTLRSCFSWSGGLGDFSRTPSPSPSTALQQFRRKSDSPTSLPENNMSDVSQLKSEESSDDESHPLREGACSSQSQESGEFSLQSSNASKLSQCSSKDSDSEESDCNIKLLDSQSDQTSKLCLSHFSKKDTPLRNKVPGLYKSSSADSLSTTKIKPLGPARASGLSKKPASIQKRKHHNAENKPGLQIKLNELWKNFGFKKFRD

SEQ ID NO: 421 - protein, 364 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 422 - protein, 364 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 423 - protein, 255 aa, synthetic construct
MWIGIISLFPEMFRAITDYGVTGRAVKNGLLSIQSWSPRDFTHDRHRTVDDRPYGGGPGMLMMVQPLRDAIHAAKAAAGEGAKVIYLSPQGRKLDQAGVSELATNQKLILVCGRYEGIDERVIQTEIDEEWSIGDYVLSGGELPAMTLIDSVSRFIPGVLGHEASATEDSFAEGLLDCPHYTRPEVLEGMEVPPVLLSGNHAEIRRWRLKQSLGRTWLRRPELLENLALTEEQARLLAEFKTEHAQQQHKHDGMA

SEQ ID NO: 424 - protein, 246 aa, synthetic construct
MWIGVISLFPEMFKAITEFGVTGRAVKHNLLKVECWNPRDFTFDKHKTVDDRPYGGGPGMLMMVQPLRDAIHTAKAAAGEGAKVIYLSPQGRKLDQGGVTELAQNQKLILVCGRYEGIDERLIQTEIDEEWSIGDYVLTGGELPAMTLIDAVARFIPGVLGKQASAEEDSFADGLLDCPHYTRPEVLEGLTVPPVLMSGHHEEIRKWRLKQSLQRTWLRRPELLEGLALTDEQRKLLKEAQAEHNS

SEQ ID NO: 425 - protein, 509 aa, synthetic construct
MVLWILWRPFGFSGRFLKLESHSITESKSLIPVAWTSLTQMLLEAPGIFLLGQRKRFSTMPETETHERETELFSPPSDVRGMTKLDRTAFKKTVNIPVLKVRKEIVSKLMRSLKRAALQRPGIRRVIEDPEDKESRLIMLDPYKIFTHDSFEKAELSVLEQLNVSPQISKYNLELTYEHFKSEEILRAVLPEGQDVTSGFSRIGHIAHLNLRDHQLSFKHLIGQVMIDKNPGITSAVNKINNIDNMYRNFQMEVLSGEQNMMTKVRENNYTYEFDFSKVYWNPRLSTEHSRITELLKPGDVLFDVFAGVGPFAIPVAKKNCTVFANDLNPESHKWLLYNCKLNKVDQKVKVFNLDGKDFLQGPVKEELMQLLGLSKERKPSVHVVMNLPAKAIEFLSAFKWLLDGQPCSSEFLPIVHCYSFSKDANPAEDVRQRAGAVLGISLEACSSVHLVRNVAPNKEMLCITFQIPASVLYKNQTRNPENHEDPPLKRQRTAEAFSDEKTQIVSNT

SEQ ID NO: 426 - protein, 336 aa, synthetic construct
MPLCLKINKKHGEQTRRILIENNLLNKDYKITSEGNYLYLPIKDVDEDILKSILNIEFELVDKELEEKKIIKKPSFREIISKKYRKEIDEGLISLSYDVVGDLVILQISDEVDEKIRKEIGELAYKLIPCKGVFRRKSEVKGEFRVRELEHLAGENRTLTIHKENGYRLWVDIAKVYFSPRLGGERARIMKKVSLNDVVVDMFAGVGPFSIACKNAKKIYAIDINPHAIELLKKNIKLNKLEHKIIPILSDVREVDVKGNRVIMNLPKFAHKFIDKALDIVEEGGVIHYYTIGKDFDKAIKLFEKKCDCEVLEKRIVKSYAPREYILALDFKINKK

SEQ ID NO: 427 - protein, 333 aa, synthetic construct
MSGVKVRREDAKKVLELLKSVGILDGKRKAIRDEKYVIFPVTDTNIAKSLGLEVVDVELPMRPERQIYKNLEDLLPREIFKKLGRLDIVGDIAIVSIPDEILSEREVIVSAIRKLYPKVKVIARRGFHSGLYRIRELEVIWGENRLHTIHKENGVLIKVDLSKVFFNPRMKGERYRIAQLVNDGERILVPFAGVIPYPLVIARFKNVEVYAVEINEFAVKLAEENLELNRDRLKGKIKIIHGDVFEVLPNLPNFDRVVSPTPKGVDALSLTLSKAEKFLHYYDFVHESEIERFRERVLEECRRQGKECRVSVRKVSDYKPHVYKVCADVEILS

SEQ ID NO: 428 - protein, 330 aa, synthetic construct
MTLAVKVPLKEGEIVRRRLIELGALDNTYKIKREGNFLLIPVKFPVKGFEVVEAELEQVSRRPNSYREIVNVPQELRRFLPTSFDIIGNIAIIEIPEELKGYAKEIGRAIVEVHKNVKAVYMKGSKIEGEYRTRELIHIAGENITETIHRENGIRLKLDVAKVYFSPRLATERMRVFKMAQEGEVVFDMFAGVGPFSILLAKKAELVFACDINPWAIKYLEENIKLNKVNNVVPILGDSREIEVKADRIIMNLPKYAHEFLEHAISCINDGGVIHYYGFGPEGDPYGWHLERIRELANKFGVKVEVLGKRVIRNYAPRQYNIAIDFRVSF

SEQ ID NO: 429 - protein, 277 aa, synthetic construct
MWSLMHPYLPATVRKWTNLNLSNHSFSLNTMKTKRLSEFFPGVRSYYIVGEIAIITPKRVNVDYNLVAEKIMQAHPKIKAVYLKKKVKGELRTNELEFLSGERISSTIYKENGVLFYVDINKVYVNPSLSGDRLKNLELVEEGSTVLDAFTGYGAIALNIAHKKRVYVVAGDINIDGLYMLKKSLSLNKIKGEMIDIVQYDAHHLPFRDKVFKLSFGDNPTLIIDFKEELCRVSENVVFYILCESEDKANSSLGRTSWIKINDYSKNLFIFKGLVRC

SEQ ID NO: 430 - protein, 468 aa, synthetic construct
MFDESKFDVNLKLWALRIPRELCKSASRILNGYMLNMPRIKPITEDPTCEKTRLVILSESVKNADLSEIPEEKLNQLKKLSELEVVPHSVTLGYSYWSADHLLKQILPDGLDIPSSFETIGHIAHLNLHDELLPFKDVIAKVIYDKNYPRIKTIVNKVGTISNEFRVPKFEVLAGENGMETEVKQYGARFKLDYGLVYWNSRLEHEHMRLSSLFKPGETVCDMFAGIGPFAIPAAQKGCFVYANDLNPDSVRYLKINAKFNKVDDLICVHNMDARKFFSHLMAVSTCEDNLQSVADNDKTKEAAVSRGGETNSSGEEIRESNASINEPLGANKKPSGTTKTENGVGKDCKSIEGHANKRLRQTLLPIAKPWEHIDHVIMNLPASALQFLDSFSNVIQKKYWKGPLPLIHCYCFIRASETTEFIIAEAETALKFHIEDPVFHKVRDVAPNKAMFCLSFRLPEACLKQEE

SEQ ID NO: 431 - protein, 499 aa, synthetic construct
MKIALPVFQKFNRLISSCKMSGVFPYNPPVNRQMRELDRSFFITKIPMCAVKFPEPKNISVFSKNFKNCILRVPRIPHVVKLNSSKPKDELTSVQNKKLKTADGNNTPVTKGVLLHESIHSVEDAYGKLPEDALAFLKENSAEIVPHEYVLDYDFWKAEEILRAVLPEQFLEEVPTGFTITGHIAHLNLRTEFKPFDSLIGQVILDKNNKIECVVDKVSSIATQFRTFPMKVIAGKSDSLVVEQKESNCTFKFDFSKVYWNSRLHTEHERLVKQYFQPGQVVCDVFAGVGPFAVPAGKKDVIVLANDLNPESYKYLKENIALNKVAKTVKSFNMDGADFIRQSPQLLQQWIQDEEGGKITIPLPLKKRHRSQQHNDQQPPQPRTKELIIPSHISHYVMNLPDSAISFLGNFRGIFAAHTKGATDTIQMPWVHVHCFEKYPPGDQVTEDELHARVHARIIAALKVTADDLPLNAVSLHLVRKVAPTKPMYCASFQLPANV

SEQ ID NO: 432 - protein, 501 aa, synthetic construct
MRIVWKLFGFSRRLLQVEWCHPSESILLFTLVPRLRKAPSVFLLGQRQGLSTMPEIEASVRDSELFSPPSDVRGMRELDRTAFKKTVSIPVLKARKEVVNRLMRALRRVALQRPGIKRVIEDPKDEDSRLIMLDPYRMLTADSFDKAELGVLKELDVSPQLSQYNLELTYENFKSEEILKAVLPEGQDVTSGFSRVGHIAHLNLRDHQLPFKHLIGQVMVDKNPGITSAVNKTSNIDNTYRNFQMEVLCGEENMLTKVRENNYTYEFDFSKVYWNPRLSTEHGRITELLNPGDVLFDVFAGVGPFAIPAARKNCTVFANDLNPESHKWLLHNCKLNKVDQKVKVFNMDGKDFIQGPVREELMLRLGLSAEAKPSVHIVMNLPAKAIEFLSVFRSLLDGQPCSTELLPTVHCYCFSKDSDPAKDVRQQAEAVLGVSLETSSSVHLVRNVAPNKEMLCITFQIPTATLYRNQSLSLQNDQEPPLKRQKTGDPFSGEPQIASDS

SEQ ID NO: 433 - protein, 293 aa, synthetic construct
MSNDEINQNEEKVKRTPPLPPVPEGMSKKQWKKMCKRQRWEENKAKYNAERRVKKKRLRHERSAKIQEYIDRGEEVPQELIREPRINVNQTDSGIEIILDCSFDELMNDKEIVSLSNQVTRAYSANRRANHFAEIKVAPFDKRLKQRFETTLKNTNYENWNHFKFLPDDKIMFGDEHISKDKIVYLTADTEEKLEKLEPGMRYIVGGIVDKNRYKELCLKKAQKMGIPTRRLPIDEYINLEGRRVLTTTHVVQLMLKYFDDHNWKNAFESVLPPRKLDAEAKSASSSPAPKDT

SEQ ID NO: 434 - protein, 304 aa, synthetic construct
MENKDALDIGKDDTNTSEADVSKNETQEQPVLSKSALKRLKRQQEWDAGREKRAEMRREKKRLRKEERKRKIEAGEVVKSQKKRIRLGKVVPSSIRIVLDCAFDDLMNDKEINSLCQQVTRCHSANRTALHPVELFATNFGGRLKTRQDFVLKGQQNNWKRYNPTTKSYLEEFESQKEKLVYLSADSDNTITELDEDKIYIIGAIVDKNRYKNLCQNKASEQGIKTAKLPIDEYIKITDRKILTVNQVFEILSLWLEYRDWEKAFMEVIPKRKGILLKSDESFDVSEDTRSQSNQSDSELEKEN

SEQ ID NO: 435 - protein, 339 aa, synthetic construct
MSSEMLPAFIETSNVDKKQGINEDQEESQKPRLGEGCEPISKRQMKKLIKQKQWEEQRELRKQKRKEKRKRKKLERQCQMEPNSDGHDRKRVRRDVVHSTLRLIIDCSFDHLMVLKDIKKLHKQIQRCYAENRRALHPVQFYLTSHGGQLKKNMDENDKGWVNWKDIHIKPEHYSELIKKEDLIYLTSDSPNILKELDESKAYVIGGLVDHNHHKGLTYKQASDYGINHAQLPLGNFVKMNSRKVLAVNHVFEIILEYLETRDWQEAFFTILPQRKGAVPTDKACESASHDNQSVRMEEGGSDSDSSEEEYSRNELDSPHEEKQDKENHTESTVNSLPH

SEQ ID NO: 436 - protein, 316 aa, synthetic construct
MDWKLEGSTQKVESPVLQGQEGILEETGEDGLPEGFQLLQIDAEGECQEGEILATGSTAWCSKNVQRKQRHWEKIVAAKKSKRKQEKERRKANRAENPGICPQHSKRFLRALTKDKLLEAKHSGPRLCIDLSMTHYMSKKELSRLAGQIRRLYGSNKKADRPFWICLTGFTTDSPLYEECVRMNDGFSSYLLDITEEDCFSLFPLETLVYLTPDSEHALEDVDLNKVYILGGLVDESIQKKVTFQKAREYSVKTARLPIQEYMVRNQNGKNYHSEILAINQVFDILSTYLETHNWPEALKKGVSSGKGYILRNSVE

SEQ ID NO: 437 - protein, 403 aa, synthetic construct
MAAFLKMSVSVNFFRPFTRFLVPFTLHRKRNNLTILQRYMSSKIPAVTYPKNESTPPSEELELDKWKTTMKSSVQEECVSTISSSKDEDPLAATREFIEMWRLLGREVPEHITEEELKTLMECVSNTAKKKYLKYLYTKEKVKKARQIKKEMKAAAREEAKNIKLLETTEEDKQKNFLFLRLWDRNMDIAMGWKGAQAMQFGQPLVFDMAYENYMKRKELQNTVSQLLESEGWNRRNVDPFHIYFCNLKIDGALHRELVKRYQEKWDKLLLTSTEKSHVDLFPKDSIIYLTADSPNVMTTFRHDKVYVIGSFVDKSMQPGTSLAKAKRLNLATECLPLDKYLQWEIGNKNLTLDQMIRILLCLKNNGNWQEALQFVPKRKHTGFLEISQHSQEFINRLKKAKT

SEQ ID NO: 438-501 - skipped (64 entries)

SEQ ID NO: 502 - protein, 6 aa, synthetic construct
  REGION >6: note = The entire sequence of amino acids 1-6 can be repeated one or more times.
GSGGSN

SEQ ID NO: 503 - protein, 7 aa, synthetic construct
  source 1..7: note = Complete motif can be repeated any number of times
  REGION >7: note = The entire sequence of amino acids 1-7 can be repeated one or more times.
GGSGGSN

SEQ ID NO: 504 - protein, 5 aa, synthetic construct
  REGION >5: note = The entire sequence of amino acids 1-5 can be repeated one or more times.
GGGSN

SEQ ID NO: 505 - protein, 4 aa, synthetic construct
GGSG

SEQ ID NO: 506 - protein, 5 aa, synthetic construct
GGSGG

SEQ ID NO: 507 - protein, 5 aa, synthetic construct
GSGSG

SEQ ID NO: 508 - protein, 5 aa, synthetic construct
GSGGG

SEQ ID NO: 509 - protein, 5 aa, synthetic construct
GGGSG

SEQ ID NO: 510 - protein, 7 aa, synthetic construct
GGGSGGS

SEQ ID NO: 511 - protein, 5 aa, synthetic construct
GSSSG

SEQ ID NO: 512 - protein, 200 aa, synthetic construct
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATP

SEQ ID NO: 513 - protein, 10 aa, synthetic construct
GSPAGSPTST

SEQ ID NO: 514 - protein, 20 aa, synthetic construct
GSPAGSPTSTEEGTSESATP

SEQ ID NO: 515 - protein, 40 aa, synthetic construct
GSPAGSPTSTEEGTSESATPESGPGTSTEPSEGSAPGSPA

SEQ ID NO: 516 - protein, 80 aa, synthetic construct
GSPAGSPTSTEEGTSESATPESGPGTSTEPSEGSAPGSPAGSPTSTEEGTSTEPSEGSAPGTSTEPSEGSAPGTSESATP

SEQ ID NO: 517 - protein, 21 aa, synthetic construct
GSGSPAGSPTSTRSGGGSGTS

SEQ ID NO: 518-529 - skipped (12 entries)

SEQ ID NO: 530 - protein, 920 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVAVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKGSGSPAGSPTSTRSGGGSGTSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 531 - protein, 920 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVAVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKGSGSPAGSPTSTRSGGGSGTSSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 532 - DNA, 56 bp, synthetic construct
  source 1..56: mol_type = other DNA
ctttcaagactaatagattgctccttacgaggagacgggccgagatgtctcgctcc

SEQ ID NO: 533 - DNA, 56 bp, synthetic construct
  source 1..56: mol_type = other DNA
ctttcaagactaatagattgctccttacgaggagacgcatgtgcaaacgccttcaa

SEQ ID NO: 534 - DNA, 56 bp, synthetic construct
  source 1..56: mol_type = other DNA
ctttcaagactaatagattgctccttacgaggagactcccacagatatccagaacc

SEQ ID NO: 535 - DNA, 56 bp, synthetic construct
  source 1..56: mol_type = other DNA
ctttcaagactaatagattgctccttacgaggagacatataagtggaggcgtcgcg

SEQ ID NO: 536 - DNA, 56 bp, synthetic construct
  source 1..56: mol_type = other DNA
ctttcaagactaatagattgctccttacgaggagactggcctggaggctatccagc

SEQ ID NO: 537 - DNA, 56 bp, synthetic construct
  source 1..56: mol_type = other DNA
ctttcaagactaatagattgctccttacgaggagacgccttaacaagctgctcttc

SEQ ID NO: 538 - DNA, 56 bp, synthetic construct
  source 1..56: mol_type = other DNA
ctttcaagactaatagattgctccttacgaggagacgcacatgcaaagtcagattt

SEQ ID NO: 539 - skipped

SEQ ID NO: 540 - DNA, 56 bp, synthetic construct
  source 1..56: mol_type = other DNA
ctttcaagactaatagattgctccttacgaggagacacacatgcccaggcagcacc

SEQ ID NO: 541 - DNA, 56 bp, synthetic construct
  source 1..56: mol_type = other DNA
ctttcaagactaatagattgctccttacgaggagacgagcagccaaggtgcccctg

SEQ ID NO: 542 - skipped

SEQ ID NO: 543 - protein, 920 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVNVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKGSGSPAGSPTSTRSGGGSGTSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 544 - protein, 920 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIANLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKGSGSPAGSPTSTRSGGGSGTSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 545 - protein, 920 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIQNLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKGSGSPAGSPTSTRSGGGSGTSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 546 - protein, 920 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNAAIDVATENLATVAITAQSMPKPTCERSGDAKKPVRARKAKAPEFHDKLAPSYTVVLREAVKRPAATKKAGQAKKKKGSGSPAGSPTSTRSGGGSGTSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 547 - protein, 920 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNANIDVATENLATVAITAQSMPKPTCERSGDAKKPVRARKAKAPEFHDKLAPSYTVVLREAVKRPAATKKAGQAKKKKGSGSPAGSPTSTRSGGGSGTSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 548 - protein, 1118 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVAVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKGSGSPAGSPTSTRSGGGSGTSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 549 - protein, 1118 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVNVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKGSGSPAGSPTSTRSGGGSGTSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 550 - protein, 1118 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIANLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKGSGSPAGSPTSTRSGGGSGTSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 551 - protein, 1118 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIQNLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKGSGSPAGSPTSTRSGGGSGTSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 552 - protein, 1118 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNAAIDVATENLATVAITAQSMPKPTCERSGDAKKPVRARKAKAPEFHDKLAPSYTVVLREAVKRPAATKKAGQAKKKKGSGSPAGSPTSTRSGGGSGTSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 553 - protein, 1118 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNANIDVATENLATVAITAQSMPKPTCERSGDAKKPVRARKAKAPEFHDKLAPSYTVVLREAVKRPAATKKAGQAKKKKGSGSPAGSPTSTRSGGGSGTSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 554 - protein, 1118 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVAVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKGSGSPAGSPTSTRSGGGSGTSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 555 - protein, 1118 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVNVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKGSGSPAGSPTSTRSGGGSGTSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 556 - protein, 1118 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIANLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKGSGSPAGSPTSTRSGGGSGTSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 557 - protein, 1118 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIQNLVKKNNFFGGSGKREPGWDNFYKPKKENRWWINAIHKALTELSQNKGKRVILLPAMRTSITCPKCKYCDSKNRNGEKFNCLKCGIELNADIDVATENLATVAITAQSMPKPTCERSGDAKKPVRARKAKAPEFHDKLAPSYTVVLREAVKRPAATKKAGQAKKKKGSGSPAGSPTSTRSGGGSGTSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 558 - protein, 1118 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNAAIDVATENLATVAITAQSMPKPTCERSGDAKKPVRARKAKAPEFHDKLAPSYTVVLREAVKRPAATKKAGQAKKKKGSGSPAGSPTSTRSGGGSGTSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 559 - protein, 1118 aa, synthetic construct
MIKPTVSQFLTPGFKLIRNHSRTAGLKLKNEGEEACKKFVRENEIPKDECPNFQGGPAIANIIAKSREFTEWEIYQSSLAIQEVIFTLPKDKLPEPILKEEWRAQWLSEHGLDTVPYKEAAGLNLIIKNAVNTYKGVQVKVDNKNKNNLAKINRKNEIAKLNGEQEISFEEIKAFDDKGYLLQKPSPNKSIYCYQSVSPKPFITSKYHNVNLPEEYIGYYRKSNEPIVSPYQFDRLRIPIGEPGYVPKWQYTFLSKKENKRRKLSKRIKNVSPILGIICIKKDWCVFDMRGLLRTNHWKKYHKPTDSINDLFDYFTGDPVIDTKANVVRFRYKMENGIVNYKPVREKKGKELLENICDQNGSCKLATVDVGQNNPVAIGLFELKKVNGELTKTLISRHPTPIDFCNKITAYRERYDKLESSIKLDAIKQLTSEQKIEVDNYNNNFTPQNTKQIVCSKLNINPNDLPWDKMISGTHFISEKAQVSNKSEIYFTSTDKGKTKDVMKSDYKWFQDYKPKLSKEVRDALSDIEWRLRRESLEFNKLSKSREQDARQLANWISSMCDVIGIENLVKKNNFFGGSGKREPGWDNFYKPKKENRWWINAIHKALTELSQNKGKRVILLPAMRTSITCPKCKYCDSKNRNGEKFNCLKCGIELNANIDVATENLATVAITAQSMPKPTCERSGDAKKPVRARKAKAPEFHDKLAPSYTVVLREAVKRPAATKKAGQAKKKKGSGSPAGSPTSTRSGGGSGTSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 560 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 561 - protein, 32 aa, synthetic construct
SGGSSGGSSGSETPGTSESATPESSGGSSGGS

SEQ ID NO: 562-599 - skipped (38 entries)

SEQ ID NO: 600 - protein, 16 aa, synthetic construct
KRPAATKKAGQAKKKK

SEQ ID NO: 601 - protein, 7 aa, synthetic construct
PKKKRKV

SEQ ID NO: 602 - protein, 9 aa, synthetic construct
LPPLERLTL

SEQ ID NO: 603 - protein, 16 aa, synthetic construct
MPKKKRKVGIHGVPAA

SEQ ID NO: 604-649 - skipped (46 entries)

SEQ ID NO: 650 - DNA, 36 bp, synthetic construct
  source 1..36: mol_type = other DNA
ctttcaagactaatagattgctccttacgaggagac

SEQ ID NO: 651 - DNA, 20 bp, synthetic construct
  source 1..20: mol_type = other DNA
attgctccttacgaggagac

SEQ ID NO: 652 - DNA, 24 bp, synthetic construct
  source 1..24: mol_type = other DNA
atagattgctccttacgaggagac

SEQ ID NO: 653 - DNA, 12 bp, synthetic construct
  source 1..12: mol_type = other DNA
gccttaacaagc

SEQ ID NO: 654 - DNA, 14 bp, synthetic construct
  source 1..14: mol_type = other DNA
gccttaacaagctg

SEQ ID NO: 655 - DNA, 16 bp, synthetic construct
  source 1..16: mol_type = other DNA
gccttaacaagctgct

SEQ ID NO: 656 - DNA, 18 bp, synthetic construct
  source 1..18: mol_type = other DNA
gccttaacaagctgctct

SEQ ID NO: 657 - DNA, 20 bp, synthetic construct
  source 1..20: mol_type = other DNA
gccttaacaagctgctcttc

SEQ ID NO: 658 - DNA, 23 bp, synthetic construct
  source 1..23: mol_type = other DNA
gccttaacaagctgctcttctaa

SEQ ID NO: 659 - DNA, 12 bp, synthetic construct
  source 1..12: mol_type = other DNA
gggccgagatgt

SEQ ID NO: 660 - DNA, 14 bp, synthetic construct
  source 1..14: mol_type = other DNA
gggccgagatgtct

SEQ ID NO: 661 - DNA, 16 bp, synthetic construct
  source 1..16: mol_type = other DNA
gggccgagatgtctcg

SEQ ID NO: 662 - DNA, 18 bp, synthetic construct
  source 1..18: mol_type = other DNA
gggccgagatgtctcgct

SEQ ID NO: 663 - DNA, 20 bp, synthetic construct
  source 1..20: mol_type = other DNA
atataagtggaggcgtcgcg

SEQ ID NO: 664 - DNA, 23 bp, synthetic construct
  source 1..23: mol_type = other DNA
gggccgagatgtctcgctccgtg

SEQ ID NO: 665 - DNA, 12 bp, synthetic construct
  source 1..12: mol_type = other DNA
acacatgcccag

SEQ ID NO: 666 - DNA, 14 bp, synthetic construct
  source 1..14: mol_type = other DNA
acacatgcccaggc

SEQ ID NO: 667 - DNA, 16 bp, synthetic construct
  source 1..16: mol_type = other DNA
acacatgcccaggcag

SEQ ID NO: 668 - DNA, 18 bp, synthetic construct
  source 1..18: mol_type = other DNA
acacatgcccaggcagca

SEQ ID NO: 669 - DNA, 20 bp, synthetic construct
  source 1..20: mol_type = other DNA
acacatgcccaggcagcacc

SEQ ID NO: 670 - DNA, 23 bp, synthetic construct
  source 1..23: mol_type = other DNA
acacatgcccaggcagcacctca

SEQ ID NO: 671 - DNA, 12 bp, synthetic construct
  source 1..12: mol_type = other DNA
gagcagccaagg

SEQ ID NO: 672 - DNA, 14 bp, synthetic construct
  source 1..14: mol_type = other DNA
gagcagccaaggtg

SEQ ID NO: 673 - DNA, 16 bp, synthetic construct
  source 1..16: mol_type = other DNA
gagcagccaaggtgcc

SEQ ID NO: 674 - DNA, 18 bp, synthetic construct
  source 1..18: mol_type = other DNA
gagcagccaaggtgcccc

SEQ ID NO: 675 - DNA, 20 bp, synthetic construct
  source 1..20: mol_type = other DNA
gagcagccaaggtgcccctg

SEQ ID NO: 676 - DNA, 23 bp, synthetic construct
  source 1..23: mol_type = other DNA
gagcagccaaggtgcccctggca

SEQ ID NO: 677 - RNA, 20 nt, synthetic construct
  source 1..20: mol_type = other RNA
ccccccactccccgctgctg

SEQ ID NO: 678 - RNA, 20 nt, synthetic construct
  source 1..20: mol_type = other RNA
aagtggcatcctctctgagc

SEQ ID NO: 679 - RNA, 20 nt, synthetic construct
  source 1..20: mol_type = other RNA
cttcctcgtctgcagccaca

SEQ ID NO: 680 - RNA, 20 nt, synthetic construct
  source 1..20: mol_type = other RNA
acttttcctgttttctctct

SEQ ID NO: 681 - RNA, 20 nt, synthetic construct
  source 1..20: mol_type = other RNA
ggttcctcggtgaccctaga

SEQ ID NO: 682 - RNA, 20 nt, synthetic construct
  source 1..20: mol_type = other RNA
gtgaccctagaaattggagc

SEQ ID NO: 683 - RNA, 20 nt, synthetic construct
  source 1..20: mol_type = other RNA
tctgcagccacagaatatgg

SEQ ID NO: 684 - RNA, 20 nt, synthetic construct
  source 1..20: mol_type = other RNA
aggagccacagagcattgag

SEQ ID NO: 685 - RNA, 20 nt, synthetic construct
  source 1..20: mol_type = other RNA
gaggagggcgggagacgagc

SEQ ID NO: 686 - RNA, 20 nt, synthetic construct
  source 1..20: mol_type = other RNA
tctcccgccctcctccgaca

SEQ ID NO: 687 - RNA, 20 nt, synthetic construct
  source 1..20: mol_type = other RNA
ccagttagagactccgcgga

SEQ ID NO: 688 - RNA, 20 nt, synthetic construct
  source 1..20: mol_type = other RNA
ctctgatctagacctagtta

SEQ ID NO: 689 - RNA, 20 nt, synthetic construct
  source 1..20: mol_type = other RNA
cgccggaagtaggacagagg

SEQ ID NO: 690 - RNA, 20 nt, synthetic construct
  source 1..20: mol_type = other RNA
aaaggagcgaggactcttca

SEQ ID NO: 691 - RNA, 20 nt, synthetic construct
  source 1..20: mol_type = other RNA
ctcctttcgatttcttgtcc

SEQ ID NO: 692 - RNA, 20 nt, synthetic construct
  source 1..20: mol_type = other RNA
atttcttgtcctgacactgg

SEQ ID NO: 693 - RNA, 20 nt, synthetic construct
  source 1..20: mol_type = other RNA
gaacaaggcaaaggctataa

SEQ ID NO: 694 - RNA, 20 nt, synthetic construct
  source 1..20: mol_type = other RNA
agttataatagtgtgtggac

SEQ ID NO: 695 - RNA, 20 nt, synthetic construct
  source 1..20: mol_type = other RNA
aatattagtgtactttagac

SEQ ID NO: 696 - RNA, 20 nt, synthetic construct
  source 1..20: mol_type = other RNA
ttgagccccttcctcgctgc

SEQ ID NO: 697 - RNA, 20 nt, synthetic construct
  source 1..20: mol_type = other RNA
aaggtacatgtgcaggatgt

SEQ ID NO: 698 - RNA, 20 nt, synthetic construct
  source 1..20: mol_type = other RNA
gcaaccagtagcccttgcgt

SEQ ID NO: 699 - RNA, 20 nt, synthetic construct
  source 1..20: mol_type = other RNA
cactttctttctttgtcctt

SEQ ID NO: 700 - RNA, 20 nt, synthetic construct
  source 1..20: mol_type = other RNA
gtgttcagtggattagaaac

SEQ ID NO: 701 - RNA, 20 nt, synthetic construct
  source 1..20: mol_type = other RNA
gagaagaagctgctacatct

SEQ ID NO: 702 - RNA, 20 nt, synthetic construct
  source 1..20: mol_type = other RNA
ttaacaaatattattagcag

SEQ ID NO: 703 - RNA, 20 nt, synthetic construct
  source 1..20: mol_type = other RNA
tcctacccccaccccatccc

SEQ ID NO: 704 - RNA, 20 nt, synthetic construct
  source 1..20: mol_type = other RNA
gagatggacaatggcccggg

SEQ ID NO: 705 - RNA, 20 nt, synthetic construct
  source 1..20: mol_type = other RNA
ctccgtgtacctctgttcct

SEQ ID NO: 706 - RNA, 20 nt, synthetic construct
  source 1..20: mol_type = other RNA
gtggagaagattgaattcag

SEQ ID NO: 707 - RNA, 20 nt, synthetic construct
  source 1..20: mol_type = other RNA
tacggggtgctctccaagaa

SEQ ID NO: 708 - RNA, 20 nt, synthetic construct
  source 1..20: mol_type = other RNA
tggggtaaaaaggacaagag

SEQ ID NO: 709 - RNA, 20 nt, synthetic construct
  source 1..20: mol_type = other RNA
aaaaggcggacgcactccgg

SEQ ID NO: 710 - RNA, 20 nt, synthetic construct
  source 1..20: mol_type = other RNA
ggggagggactccgtccaga

SEQ ID NO: 711 - RNA, 20 nt, synthetic construct
  source 1..20: mol_type = other RNA
gagaccatattctgtggctg

SEQ ID NO: 712 - RNA, 20 nt, synthetic construct
  source 1..20: mol_type = other RNA
aggtgtataggtggaaagac

SEQ ID NO: 713 - RNA, 20 nt, synthetic construct
  source 1..20: mol_type = other RNA
ttctcttcgggttcctcggt

SEQ ID NO: 714 - RNA, 20 nt, synthetic construct
  source 1..20: mol_type = other RNA
gagcaaattacgattgaagt

SEQ ID NO: 715 - RNA, 20 nt, synthetic construct
  source 1..20: mol_type = other RNA
cgattgaagtttagaaacat

SEQ ID NO: 716 - RNA, 20 nt, synthetic construct
  source 1..20: mol_type = other RNA
aagtttagaaacatggttgg

SEQ ID NO: 717 - RNA, 20 nt, synthetic construct
  source 1..20: mol_type = other RNA
tcggaggagggcgggagacg

SEQ ID NO: 718 - RNA, 20 nt, synthetic construct
  source 1..20: mol_type = other RNA
atctctcctgcgggtaaaaa

SEQ ID NO: 719 - RNA, 20 nt, synthetic construct
  source 1..20: mol_type = other RNA
gcttttcccttccttccctc

SEQ ID NO: 720 - RNA, 20 nt, synthetic construct
  source 1..20: mol_type = other RNA
acattagcttttcccttcct

SEQ ID NO: 721 - RNA, 20 nt, synthetic construct
  source 1..20: mol_type = other RNA
actaggtctagatcagagcg

SEQ ID NO: 722 - RNA, 20 nt, synthetic construct
  source 1..20: mol_type = other RNA
gcgccaaaggatggcttctc

SEQ ID NO: 723 - RNA, 20 nt, synthetic construct
  source 1..20: mol_type = other RNA
ggagaagccatcctttggcg

SEQ ID NO: 724 - RNA, 20 nt, synthetic construct
  source 1..20: mol_type = other RNA
gggaactaatctcaacgctg

SEQ ID NO: 725 - RNA, 20 nt, synthetic construct
  source 1..20: mol_type = other RNA
gtcaagtttgccttgtcaag

SEQ ID NO: 726 - RNA, 20 nt, synthetic construct
  source 1..20: mol_type = other RNA
gccagccttgccttgaccaa

SEQ ID NO: 727 - RNA, 20 nt, synthetic construct
  source 1..20: mol_type = other RNA
gtcaaggcaaggctggccaa

SEQ ID NO: 728 - RNA, 20 nt, synthetic construct
  source 1..20: mol_type = other RNA
agatagtgtggggaaggggc

SEQ ID NO: 729 - RNA, 20 nt, synthetic construct
  source 1..20: mol_type = other RNA
gcagtggtttctaaggaaaa

SEQ ID NO: 730 - RNA, 20 nt, synthetic construct
  source 1..20: mol_type = other RNA
gagaaaaactggaatgactg

SEQ ID NO: 731 - RNA, 20 nt, synthetic construct
  source 1..20: mol_type = other RNA
gtacatgctttagctttaaa

SEQ ID NO: 732 - RNA, 20 nt, synthetic construct
  source 1..20: mol_type = other RNA
agagataatggcaaaagtca

SEQ ID NO: 733 - RNA, 20 nt, synthetic construct
  source 1..20: mol_type = other RNA
gttcggagaagaagctgcta

SEQ ID NO: 734 - RNA, 20 nt, synthetic construct
  source 1..20: mol_type = other RNA
tgcgggggaagatgtagcag

SEQ ID NO: 735 - RNA, 20 nt, synthetic construct
  source 1..20: mol_type = other RNA
tcttttagaatttgggagcc

SEQ ID NO: 736 - RNA, 20 nt, synthetic construct
  source 1..20: mol_type = other RNA
ggtcattgtcttttagaatt

SEQ ID NO: 737 - RNA, 20 nt, synthetic construct
  source 1..20: mol_type = other RNA
tgggggagggccggagctgg

SEQ ID NO: 738 - RNA, 20 nt, synthetic construct
  source 1..20: mol_type = other RNA
tgcgtgtggggagggggtgt

SEQ ID NO: 739 - RNA, 20 nt, synthetic construct
  source 1..20: mol_type = other RNA
ggggagggaggtgtgagcct

SEQ ID NO: 740 - RNA, 20 nt, synthetic construct
  source 1..20: mol_type = other RNA
gccagcgccgccaggctcac

SEQ ID NO: 741 - RNA, 20 nt, synthetic construct
  source 1..20: mol_type = other RNA
gggagtgggtgggaggaaga

SEQ ID NO: 742 - RNA, 20 nt, synthetic construct
  source 1..20: mol_type = other RNA
caaggagcgggagaaaggaa

SEQ ID NO: 743 - RNA, 20 nt, synthetic construct
  source 1..20: mol_type = other RNA
ttgttgcagtgcgtgcgcct

SEQ ID NO: 744 - RNA, 20 nt, synthetic construct
  source 1..20: mol_type = other RNA
ttcagccgggagtccggcac

SEQ ID NO: 745 - RNA, 20 nt, synthetic construct
  source 1..20: mol_type = other RNA
ggaagggggtggggggcgtc

SEQ ID NO: 746 - RNA, 20 nt, synthetic construct
  source 1..20: mol_type = other RNA
tccctcctgtgacgcccccc

SEQ ID NO: 747 - RNA, 20 nt, synthetic construct
  source 1..20: mol_type = other RNA
cttcaagttcttagtagaat

SEQ ID NO: 748 - RNA, 20 nt, synthetic construct
  source 1..20: mol_type = other RNA
ggcagaaaaaatggcacagg

SEQ ID NO: 749 - RNA, 20 nt, synthetic construct
  source 1..20: mol_type = other RNA
tatgccgcggagcgctccat

SEQ ID NO: 750 - RNA, 20 nt, synthetic construct
  source 1..20: mol_type = other RNA
tggccagaagaaagtggccc

SEQ ID NO: 751 - RNA, 20 nt, synthetic construct
  source 1..20: mol_type = other RNA
cccgcaggagagattaaccc

SEQ ID NO: 752 - RNA, 20 nt, synthetic construct
  source 1..20: mol_type = other RNA
agggaaggaagggaaaagct

SEQ ID NO: 753 - RNA, 20 nt, synthetic construct
  source 1..20: mol_type = other RNA
gcgccaatgatcaaagcgtc

SEQ ID NO: 754 - RNA, 20 nt, synthetic construct
  source 1..20: mol_type = other RNA
atcattggcgccaaaggatg

SEQ ID NO: 755 - RNA, 20 nt, synthetic construct
  source 1..20: mol_type = other RNA
cttagaggggccgacggaga

SEQ ID NO: 756 - RNA, 20 nt, synthetic construct
  source 1..20: mol_type = other RNA
tatggcgtatatgtttgctt

SEQ ID NO: 757 - RNA, 20 nt, synthetic construct
  source 1..20: mol_type = other RNA
ttcttcatccctagccagcc

SEQ ID NO: 758 - RNA, 20 nt, synthetic construct
  source 1..20: mol_type = other RNA
ccttgtcaaggctattggtc

SEQ ID NO: 759 - RNA, 20 nt, synthetic construct
  source 1..20: mol_type = other RNA
gccagggaccgtttcagaca

SEQ ID NO: 760 - RNA, 20 nt, synthetic construct
  source 1..20: mol_type = other RNA
cattgagatagtgtggggaa

SEQ ID NO: 761 - RNA, 20 nt, synthetic construct
  source 1..20: mol_type = other RNA
tagcctttgccttgttccga

SEQ ID NO: 762 - RNA, 20 nt, synthetic construct
  source 1..20: mol_type = other RNA
ccttgttccgattcagtcat

SEQ ID NO: 763 - RNA, 20 nt, synthetic construct
  source 1..20: mol_type = other RNA
ttcttccctttagctagttt

SEQ ID NO: 764 - RNA, 20 nt, synthetic construct
  source 1..20: mol_type = other RNA
gctagtttccttctcccatc

SEQ ID NO: 765 - RNA, 20 nt, synthetic construct
  source 1..20: mol_type = other RNA
aaaagccgtgggccctccca

SEQ ID NO: 766 - RNA, 20 nt, synthetic construct
  source 1..20: mol_type = other RNA
ggagcctttgaaaagccgtg

SEQ ID NO: 767 - RNA, 20 nt, synthetic construct
  source 1..20: mol_type = other RNA
gaatttgggagcctttgaaa

SEQ ID NO: 768 - RNA, 20 nt, synthetic construct
  source 1..20: mol_type = other RNA
acaccccctccccacacgca

SEQ ID NO: 769 - RNA, 20 nt, synthetic construct
  source 1..20: mol_type = other RNA
agatcctgcactttggactc

SEQ ID NO: 770 - RNA, 20 nt, synthetic construct
  source 1..20: mol_type = other RNA
gggacccctactgagtccaa

SEQ ID NO: 771 - RNA, 20 nt, synthetic construct
  source 1..20: mol_type = other RNA
gactcagtaggggtccccaa

SEQ ID NO: 772 - RNA, 20 nt, synthetic construct
  source 1..20: mol_type = other RNA
aaactaccccccccaccagg

SEQ ID NO: 773 - RNA, 56 nt, synthetic construct
  source 1..56: mol_type = other RNA
ctttcaagactaatagattgctccttacgaggagacgggccgagatgtctcgctcc

SEQ ID NO: 774 - RNA, 56 nt, synthetic construct
  source 1..56: mol_type = other RNA
ctttcaagactaatagattgctccttacgaggagacgcatgtgcaaacgccttcaa

SEQ ID NO: 775 - RNA, 56 nt, synthetic construct
  source 1..56: mol_type = other RNA
ctttcaagactaatagattgctccttacgaggagactcccacagatatccagaacc

SEQ ID NO: 776 - RNA, 56 nt, synthetic construct
  source 1..56: mol_type = other RNA
ctttcaagactaatagattgctccttacgaggagacatataagtggaggcgtcgcg

SEQ ID NO: 777 - RNA, 56 nt, synthetic construct
  source 1..56: mol_type = other RNA
ctttcaagactaatagattgctccttacgaggagactggcctggaggctatccagc

SEQ ID NO: 778 - RNA, 56 nt, synthetic construct
  source 1..56: mol_type = other RNA
ctttcaagactaatagattgctccttacgaggagacgccttaacaagctgctcttc

SEQ ID NO: 779 - RNA, 56 nt, synthetic construct
  source 1..56: mol_type = other RNA
ctttcaagactaatagattgctccttacgaggagacgcacatgcaaagtcagattt

SEQ ID NO: 780 - skipped

SEQ ID NO: 781 - RNA, 56 nt, synthetic construct
  source 1..56: mol_type = other RNA
ctttcaagactaatagattgctccttacgaggagacacacatgcccaggcagcacc

SEQ ID NO: 782 - RNA, 56 nt, synthetic construct
  source 1..56: mol_type = other RNA
ctttcaagactaatagattgctccttacgaggagacgagcagccaaggtgcccctg

SEQ ID NO: 783 - RNA, 36 nt, synthetic construct
  source 1..36: mol_type = other RNA
ctttcaagactaatagattgctccttacgaggagac

SEQ ID NO: 784 - RNA, 20 nt, synthetic construct
  source 1..20: mol_type = other RNA
attgctccttacgaggagac

SEQ ID NO: 785 - RNA, 24 nt, synthetic construct
  source 1..24: mol_type = other RNA
atagattgctccttacgaggagac

SEQ ID NO: 786 - RNA, 12 nt, synthetic construct
  source 1..12: mol_type = other RNA
gccttaacaagc

SEQ ID NO: 787 - RNA, 14 nt, synthetic construct
  source 1..14: mol_type = other RNA
gccttaacaagctg

SEQ ID NO: 788 - RNA, 16 nt, synthetic construct
  source 1..16: mol_type = other RNA
gccttaacaagctgct

SEQ ID NO: 789 - RNA, 18 nt, synthetic construct
  source 1..18: mol_type = other RNA
gccttaacaagctgctct

SEQ ID NO: 790 - RNA, 20 nt, synthetic construct
  source 1..20: mol_type = other RNA
gccttaacaagctgctcttc

SEQ ID NO: 791 - RNA, 23 nt, synthetic construct
  source 1..23: mol_type = other RNA
gccttaacaagctgctcttctaa

SEQ ID NO: 792 - RNA, 12 nt, synthetic construct
  source 1..12: mol_type = other RNA
gggccgagatgt

SEQ ID NO: 793 - RNA, 14 nt, synthetic construct
  source 1..14: mol_type = other RNA
gggccgagatgtct

SEQ ID NO: 794 - RNA, 16 nt, synthetic construct
  source 1..16: mol_type = other RNA
gggccgagatgtctcg

SEQ ID NO: 795 - RNA, 18 nt, synthetic construct
  source 1..18: mol_type = other RNA
gggccgagatgtctcgct

SEQ ID NO: 796 - RNA, 20 nt, synthetic construct
  source 1..20: mol_type = other RNA
atataagtggaggcgtcgcg

SEQ ID NO: 797 - RNA, 23 nt, synthetic construct
  source 1..23: mol_type = other RNA
gggccgagatgtctcgctccgtg

SEQ ID NO: 798 - RNA, 12 nt, synthetic construct
  source 1..12: mol_type = other RNA
acacatgcccag

SEQ ID NO: 799 - RNA, 14 nt, synthetic construct
  source 1..14: mol_type = other RNA
acacatgcccaggc

SEQ ID NO: 800 - RNA, 16 nt, synthetic construct
  source 1..16: mol_type = other RNA
acacatgcccaggcag

SEQ ID NO: 801 - RNA, 18 nt, synthetic construct
  source 1..18: mol_type = other RNA
acacatgcccaggcagca

SEQ ID NO: 802 - RNA, 20 nt, synthetic construct
  source 1..20: mol_type = other RNA
acacatgcccaggcagcacc

SEQ ID NO: 803 - RNA, 23 nt, synthetic construct
  source 1..23: mol_type = other RNA
acacatgcccaggcagcacctca

SEQ ID NO: 804 - skipped

SEQ ID NO: 805 - RNA, 14 nt, synthetic construct
  source 1..14: mol_type = other RNA
gagcagccaaggtg

SEQ ID NO: 806 - RNA, 16 nt, synthetic construct
  source 1..16: mol_type = other RNA
gagcagccaaggtgcc

SEQ ID NO: 807 - RNA, 18 nt, synthetic construct
  source 1..18: mol_type = other RNA
gagcagccaaggtgcccc

SEQ ID NO: 808 - RNA, 20 nt, synthetic construct
  source 1..20: mol_type = other RNA
gagcagccaaggtgcccctg

SEQ ID NO: 809 - RNA, 23 nt, synthetic construct
  source 1..23: mol_type = other RNA
gagcagccaaggtgcccctggca

SEQ ID NO: 810 - DNA, 19 bp, synthetic construct
  source 1..19: mol_type = other DNA
acatgaggatcacccatgt

SEQ ID NO: 811 - DNA, 25 bp, synthetic construct
  source 1..25: mol_type = other DNA
ggagcagacgatatggcgtcgctcc

SEQ ID NO: 812 - DNA, 15 bp, synthetic construct
  source 1..15: mol_type = other DNA
gccctgaagaagggc

SEQ ID NO: 813 - DNA, 40 bp, synthetic construct
  source 1..40: mol_type = other DNA
tttacacatgcccaggcagcacctcaggccctttgtgggg

SEQ ID NO: 814 - DNA, 40 bp, synthetic construct
  source 1..40: mol_type = other DNA
tttacacgtgcccaggcagcacctcaggccctttgtgggg

SEQ ID NO: 815 - DNA, 40 bp, synthetic construct
  source 1..40: mol_type = other DNA
tttacacgtgcccgggcagcacctcaggccctttgtgggg

SEQ ID NO: 816 - DNA, 40 bp, synthetic construct
  source 1..40: mol_type = other DNA
tttacacgtgcccaggcagcgcctcaggccctttgtgggg

SEQ ID NO: 817 - DNA, 40 bp, synthetic construct
  source 1..40: mol_type = other DNA
tttacacatgcccaggcagcgcctcaggccctttgtgggg

SEQ ID NO: 818 - DNA, 40 bp, synthetic construct
  source 1..40: mol_type = other DNA
tttacacatgcccaggcagcacctcgggccctttgtgggg

SEQ ID NO: 819 - DNA, 40 bp, synthetic construct
  source 1..40: mol_type = other DNA
tttacacatgcccaggcagcacctcaggccctttgcgggg

SEQ ID NO: 820 - DNA, 37 bp, synthetic construct
  source 1..37: mol_type = other DNA
tttacacgtgcccaggcacctcaggccctttgtgggg

SEQ ID NO: 821 - DNA, 40 bp, synthetic construct
  source 1..40: mol_type = other DNA
tttacacgtgcccgggcagcgcctcaggccctttgtgggg

SEQ ID NO: 822 - DNA, 40 bp, synthetic construct
  source 1..40: mol_type = other DNA
tttacacatgcccaggcagcaccccaggccctttgtgggg

SEQ ID NO: 823 - DNA, 37 bp, synthetic construct
  source 1..37: mol_type = other DNA
tttacacatgcccaggcagctcaggccctttgtgggg

SEQ ID NO: 824 - DNA, 40 bp, synthetic construct
  source 1..40: mol_type = other DNA
tttacacatgcccgggcagcacctcaggccctttgtgggg

SEQ ID NO: 825 - DNA, 37 bp, synthetic construct
  source 1..37: mol_type = other DNA
tttacacatgcccaggcacctcaggccctttgtgggg

SEQ ID NO: 826 - DNA, 40 bp, synthetic construct
  source 1..40: mol_type = other DNA
tttacacatgcccaggcggcacctcaggccctttgtgggg

SEQ ID NO: 827 - DNA, 40 bp, synthetic construct
  source 1..40: mol_type = other DNA
tctacacatgcccaggcagcacctcaggccctttgtgggg

SEQ ID NO: 828 - DNA, 40 bp, synthetic construct
  source 1..40: mol_type = other DNA
tttacacatgctcaggcagcacctcaggccctttgtgggg

SEQ ID NO: 829 - DNA, 40 bp, synthetic construct
  source 1..40: mol_type = other DNA
tttacacatgcccaggcagcacctcaggccccttgtgggg

SEQ ID NO: 830 - DNA, 34 bp, synthetic construct
  source 1..34: mol_type = other DNA
tttacacatgcccaacctcaggccctttgtgggg

SEQ ID NO: 831 - DNA, 40 bp, synthetic construct
  source 1..40: mol_type = other DNA
tttacacatgtccaggcagcacctcaggccctttgtgggg

SEQ ID NO: 832 - DNA, 40 bp, synthetic construct
  source 1..40: mol_type = other DNA
cttacacatgcccaggcagcacctcaggccctttgtgggg

(19) United States
(12) Patent Application Publication
(10) Pub. No.: US 2024/0327812 A1
(43) Pub. Date: Oct. 3, 2024

(54) FUSION EFFECTOR PROTEINS AND USES THEREOF

(71) Applicant: Mammoth Biosciences, Inc., Brisbane, CA (US)

(72) Inventors: Lucas Benjamin HARRINGTON, San Francisco, CA (US); Yuchen GAO, South San Francisco, CA (US); Yuxuan ZHENG, San Jose, CA (US)

(21) Appl. No.: 18/632,018

(22) Filed: Apr. 10, 2024

Related U.S. Application Data
(63) Continuation of application No. PCT/US2022/078147, filed on Oct. 14, 2022.
(60) Provisional application No. 63/256,386, filed on Oct. 15, 2021.
(60) Provisional application No. 63/282,931, filed on Nov. 24, 2021.
(60) Provisional application No. 63/290,536, filed on Dec. 16, 2021.
(60) Provisional application No. 63/316,340, filed on Mar. 3, 2022.
(60) Provisional application No. 63/373,663, filed on Aug. 26, 2022.
(60) Provisional application No. 63/371,310, filed on Aug. 12, 2022.
(60) Provisional application No. 63/373,661, filed on Aug. 26, 2022.

(51) Int. Cl. C12N 9/22; C12N 9/10; C12N 9/78; C12N 15/11; C12N 15/86

(52) U.S. Cl. CPC C12N 9/22; C12N 9/1007; C12N 9/78; C12N 15/111; C12N 15/86; C07K 2319/80; C12N 2750/14143; C12Y 201/01037; C12Y 305/04002

(57) ABSTRACT

The present disclosure provides compositions of CRISPR associated (Cas) effector proteins fused to partner proteins. Compositions typically comprise a guide nucleic acid. Also disclosed are the methods and systems for detecting and modifying target nucleic acids using the same. The cells, progenies thereof, and populations thereof produced by the compositions, methods, or systems provided herein are also described.

The present disclosure relates generally to compositions of effector proteins, and more specifically to effector proteins fused to partner proteins, including base editors, and methods and systems of using such compositions, including detecting and editing target nucleic acids.

### CROSS REFERENCE TO RELATED APPLICATIONS

This application is a continuation of International Patent Application No. PCT/US2022/078147, filed Oct. 14, 2022, which claims the benefit of U.S. Provisional Application No. 63/256,386, filed Oct. 15, 2021, U.S. Provisional Application No. 63/282,931, filed Nov. 24, 2021, U.S. Provisional Application No. 63/290,536, filed Dec. 16, 2021, U.S. Provisional Application No. 63/316,340, filed Mar. 3, 2022, U.S. Provisional Application No. 63/373,663, filed Aug. 26, 2022, U.S. Provisional Application No. 63/371,310, filed Aug. 12, 2022, and U.S. Provisional Application No. 63/373,661, filed Aug. 26, 2022, the disclosures of each of which are incorporated herein by reference in their entirety.

### SEQUENCE LISTING

The instant application contains a Sequence Listing, which has been submitted via Patent Center. The Sequence Listing titled 203477-701301US_SL.xml, which was created on Mar. 21, 2024, and is 685,921 bytes in size, is hereby incorporated by reference in its entirety.

### BACKGROUND

Programmable CRISPR-associated (Cas) nucleases, through their ability to generate a double-stranded DNA break (DSB) at a precise target location in the genome of a wide variety of cells and organisms, allow for precise and efficient editing of DNA sequences of interest. Although DSBs are an effective way to disrupt a gene of interest, more reliable techniques that generate precise DNA or RNA modifications are necessary to make comparisons between alleles, study the effects of specific mutations within genes, and to treat genetic disease through gene correction. For instance, the largest class of known human pathogenic mutations, by far, is the point mutation (also called single nucleotide polymorphism (SNP)). Correcting pathogenic SNPs efficiently is of great interest for the study and treatment of genetic disorders, but is complicated by the possibility of undesired byproducts when attempting to change a single nucleotide.

Base editing is a genome editing method that directly generates precise nucleotide changes in genomic DNA or RNA without generating DSBs, requiring a DNA donor template, or relying on cellular homology-directed repair (HDR). In some instances, base editors comprise a base editing enzyme (e.g., a deaminase) fused to a catalytically inactive CRISPR-associated (Cas) protein, wherein the catalytically inactive CRISPR-associated (Cas) protein is coupled to a guide nucleic acid that imparts activity or sequence selectivity to the base editor.

In addition to base editors, additional types of fusion proteins with programmable nucleases may be useful for modulating gene expression. Programmable Cas nucleases, also referred to simply as programmable nucleases, may be utilized to initiate or increase gene expression, e.g., by fusion of the programmable Cas nuclease to a transcriptional activator. Similarly, programmable Cas nucleases may be utilized to arrest or reduce gene expression, e.g., by fusion of the programmable Cas nuclease to a transcriptional repressor. In general, the programmable nucleases utilized in such fusion proteins have been modified relative to a wildtype nuclease to reduce or abolish any inherent nuclease activity.

### SUMMARY

The present disclosure provides for compositions and systems comprising a fusion effector protein and uses thereof. Accordingly, in one aspect, provided herein are compositions comprising a fusion effector protein. Such a fusion effector protein can, in some embodiments, comprise a fusion partner protein and an effector protein. The effector protein of the fusion effector protein can, in some embodiments, comprise an amino acid sequence that is at least 75%, at least 80%, at least 85%, at least 90%, at least 95%, at least 98%, at least 99%, or 100% identical to any one of the fusion effector proteins disclosed herein (e.g., SEQ ID NOs: 1-226). Non-limiting examples of fusion partner proteins are base editing enzymes, prime editing enzymes, transcriptional activators, transcriptional inhibitors and transposases. In some embodiments, the effector protein of the fusion effector protein comprises an amino acid sequence that is at least 75%, at least 80%, at least 85%, at least 90%, at least 95%, at least 98%, at least 99%, or 100% identical to any one of the sequences recited in TABLE 1. The fusion partner protein of the fusion effector protein can, in some embodiments, comprise an amino acid sequence that is at least 75%, at least 80%, at least 85%, at least 90%, at least 95%, at least 98%, at least 99%, or 100% identical to any one of fusion partner proteins disclosed herein (e.g., SEQ ID NOs: 400-422). In some embodiments, the fusion partner protein of the fusion effector protein comprises an amino acid sequence that is at least 75%, at least 80%, at least 85%, at least 90%, at least 95%, at least 98%, at least 99%, or 100% identical to any one of the sequences recited in TABLE 2.

In some embodiments, the compositions disclosed herein comprise a fusion effector protein comprising a base editing enzyme. In some embodiments, such a base editing enzyme makes a nucleobase modification selected from: a cytosine to a guanine, a cytosine to a thymine, or a guanine to an adenine. In some embodiments, such a base editing enzyme makes a nucleobase modification of an adenine to a guanine. In some embodiments, the base editing enzyme comprises a deaminase or an enzyme with deaminase activity. In some embodiments, the deaminase or enzyme with deaminase activity is selected from ABE8e, ABE8.20m, APOBEC3A, AncAPOBEC, and BtAPOBEC2, and a functional fragment thereof. In some embodiments, the deaminase or enzyme with deaminase activity comprises a sequence that is at least 75%, at least 80%, at least 85%, at least 90%, at least 95%, at least 99% or 100% identical to any one of the deaminases or enzymes with deaminase activity discloses herein (e.g., SEQ ID NOS: 400-404 and 421-422).

In some embodiments, the compositions disclosed herein comprise a fusion effector protein comprising a prime editing enzyme. In some embodiments, such a prime editing enzyme is an M-MLV RT enzyme. In some embodiments, the M-HLV RT enzyme comprises at least one mutation selected from D200N, L603W, T330P, T306K, and W313F relative to wildtype M-MLV RT enzyme. In some embodiments, the M-MLV RT enzyme comprises a sequence that is at least 75%, at least 80%, at least 85%, at least 90%, at least 95%, at least 99% or 100% identical to SEQ ID NO: 405.

In some embodiments, the compositions disclosed herein comprise a fusion effector protein comprising a transcriptional activator. In some embodiments, such a transcriptional activator is selected from TET1, TET2, P300, VPR, and VP64, and a functional fragment thereof. In some embodiments, the transcriptional activator comprises a sequence that is at least 75%, at least 80%, at least 85%, at least 90%, at least 95%, at least 98%, at least 99%, or 100% identical to any one of the transcriptional activators disclosed herein (e.g., SEQ ID NOs: 406-409 and 412).

In some embodiments, the compositions disclosed herein comprise a fusion effector protein comprising a transcriptional inhibitor. In some embodiments, such a transcriptional inhibitor is selected from DNMT3A, DNMT3L, EZH2, KRAB/KOX1, and ZIM3, and a functional fragment thereof. In some embodiments, the transcriptional inhibitor comprises a sequence that is at least 75%, at least 80%, at least 85%, at least 90%, at least 95%, at least 98%, at least 99%, or 100% identical to any one of the transcriptional inhibitors disclosed herein (e.g., SEQ ID NOs: 410-411 and 413-415).

In some embodiments, the compositions disclosed herein comprise a fusion effector protein comprising a transposase. In some embodiments, the transposase is selected from Tn5 transposase, SB100X, Phage-encoded serine integrases/recombinase 2, Phage-encoded serine integrases/recombinase 13, Human WT Exonuclease 1a, and a functional fragment thereof. In some embodiments, the transposase comprises an amino acid sequence that is at least 75%, at least 80%, at least 85%, at least 90%, at least 95%, at least 98%, at least 99%, or 100% identical to any one of the transposases disclosed herein (e.g., SEQ ID NOs: 416-420).

In some embodiments, the compositions disclosed herein comprise a fusion effector protein comprising a DNA alkylating fusion partner protein. In some embodiments, the DNA alkylating fusion partner protein is a methyl transferase fusion partner protein. In some embodiments, the DNA alkylating fusion partner protein is selected from TrmD, Trm5, Trm10, TrmT5, TrmT10, RsmE, BMT5, and BMT6. In some embodiments, the TrmD comprises an amino acid sequence that is at least 75%, at least 80%, at least 85%, at least 90%, at least 95%, at least 98%, at least 99%, or 100% identical to any one of SEQ ID NOs: 423-424. In some embodiments, the Trm5 comprises an amino acid sequence that is at least 75%, at least 80%, at least 85%, at least 90%, at least 95%, at least 98%, at least 99%, or 100% identical to any one of SEQ ID NOs: 426-431. In some embodiments, the TrmT5 comprises an amino acid sequence that is at least 75%, at least 80%, at least 85%, at least 90%, at least 95%, at least 98%, at least 99%, or 100% identical to any one of SEQ ID NOs: 425 and 432. In some embodiments, the Trm10 comprises an amino acid sequence that is at least 75%, at least 80%, at least 85%, at least 90%, at least 95%, at least 98%, at least 99%, or 100% identical to any one of SEQ ID NOs: 433-434. In some embodiments, the TrmT10 comprises an amino acid sequence that is at least 75%, at least 80%, at least 85%, at least 90%, at least 95%, at least 98%, at least 99%, or 100% identical to any one of SEQ ID NOs: 435-437.

In some embodiments, the compositions disclosed herein comprise a guide nucleic acid. In some embodiments, the guide nucleic acid is a guide RNA. In some embodiments, the guide nucleic acid comprises a sequence of any one of SEQ ID NOs: 650-652 combined with a sequence of any one of SEQ ID NOs: 653-676. In some embodiments, the guide nucleic acid comprises a sequence of any one of SEQ ID NOs: 783-785 combined with a sequence of any one of SEQ ID NOs: 786-809. In some embodiments, the guide nucleic acid comprises a sequence of any one of SEQ ID NOs: 532-538 and 540-541. In some embodiments, the guide nucleic acid comprises a sequence of any one of SEQ ID NOs: 773-779 and 781-782. In some embodiments, the guide nucleic acid comprises a spacer region of 18-20 nucleosides in length. In some embodiments, the guide nucleic acid comprises a spacer region of 18 linked nucleosides in length. In some embodiments, the guide nucleic acid comprises a sequence of any one of SEQ ID NOs: 650-652 combined with a sequence of any one of SEQ ID NOs: 656, 662, 668, or 674. In some embodiments, the guide nucleic acid comprises a sequence of any one of SEQ ID NOs: 783-785 combined with a sequence of any one of SEQ ID NOs: 789, 795, 801, or 807. In some embodiments, the guide nucleic acid comprises a spacer region of 19 linked nucleosides in length. In some embodiments, the guide nucleic acid comprises a spacer region of 20 linked nucleosides in length. In some embodiments, the guide nucleic acid comprises a sequence of any one of SEQ ID NOs: 650-652 combined with a sequence of any one of SEQ ID NOs: 657, 663, 669, or 675. In some embodiments, the guide nucleic acid comprises a sequence of any one of SEQ ID NOs: 783-785 combined with a sequence of any one of SEQ ID NOs: 790, 796, 802, or 808. In some embodiments, the guide nucleic acid does not comprise a tracrRNA.

In some embodiments, the compositions disclosed herein comprise a linker that links the effector protein to the fusion partner protein. In some embodiments, the linker comprises an amide bond, an amino acid, a peptide, a nucleotide, a polymer, or a combination thereof. In some embodiments, the linker comprises an amino acid sequence selected from any one of SEQ ID NOs: 500-517. In some embodiments, the amino terminus of the fusion partner protein is linked to the carboxy terminus of the effector protein via the linker. In some embodiments, the carboxy terminus of the fusion partner protein is linked to the amino terminus of the effector protein via the linker.

In some embodiments, the compositions disclosed herein comprise a fusion effector protein, wherein the fusion effector protein does not comprise a uracil glycosylase inhibitor (UGI), or a functional fragment thereof.

In some embodiments, the compositions disclosed herein comprise a fusion effector protein, wherein the amino acid sequence of the fusion effector protein is at least 75%, at least 80%, at least 85%, at least 90%, at least 95%, at least 98%, at least 99%, or 100% identical to any one of the fusion effector proteins described herein. For example, in some embodiments, the fusion effector protein is at least 75%, at least 80%, at least 85%, at least 90%, at least 95%, at least 98%, at least 99%, or 100% identical to SEQ ID NO: 530 or 531. In some embodiments, the fusion effector protein is at least 75%, at least 80%, at least 85%, at least 90%, at least 95%, at least 98%, at least 99%, or 100% identical to any one of SEQ ID NOs: 543-559.

In some embodiments, the compositions disclosed herein comprise a fusion effector protein, wherein the fusion effector protein comprises a fusion partner protein and an effector protein, wherein the effector protein is 350 to 400, 400 to 450, 450 to 500, 500 to 550, 550 to 600, 600 to 650, 650 to 700, 700 to 750, 750 to 800, 800 to 850, or 850 to 900 linked amino acids in length. In some embodiments, the compositions disclosed herein comprise a fusion effector protein, wherein the fusion effector protein comprises a single type of nuclease domain, wherein the single type of nuclease domain is a RuvC domain. In some embodiments, the compositions disclosed herein comprise a fusion effector protein, wherein the fusion effector protein does not comprise a zinc finger domain. In some embodiments, the compositions disclosed herein comprise a fusion effector protein, wherein the fusion effector protein does not comprise an HNH domain. In some embodiments, the effector protein is a Cas14 protein. In some embodiments, the effector protein functions as a homodimer at least when it is not fused to the fusion partner protein. In some embodiments, the effector protein is a catalytically inactive effector protein. In some embodiments, the effector protein comprises an amino acid sequence that is at least 75%, at least 80%, at least 85%, at least 90%, at least 95%, at least 98%, at least 99%, or 100% identical to any one of SEQ ID NOs: 7, 217-226.

In some embodiments, the effector protein comprises an amino acid substitution of D369A. In some embodiments, the effector protein has at least 75% sequence identity to the amino acid sequence of SEQ ID NO: 7. In some embodiments, the effector protein is at least 75%, at least 80%, at least 85%, at least 90%, at least 95%, at least 98%, or at least 99% identical to the amino acid sequence of SEQ ID NO: 7 and comprises an amino acid substitution of D369A. In some embodiments, the effector protein comprises an amino acid substitution of D369N. In some embodiments, the effector protein is at least 75%, at least 80%, at least 85%, at least 90%, at least 95%, at least 98%, or at least 99% identical to the amino acid sequence of SEQ ID NO: 217. In some embodiments, the effector protein is at least 75%, at least 80%, at least 85%, at least 90%, at least 95%, at least 98%, or at least 99% identical to the amino acid sequence of SEQ ID NO: 217 and comprises an amino acid substitution of D369N. In some embodiments, the effector protein comprises an amino acid substitution of E567A. In some embodiments, the effector protein is at least 75%, at least 80%, at least 85%, at least 90%, at least 95%, at least 98%, or at least 99% identical to the amino acid sequence of SEQ ID NO: 218. In some embodiments, the effector protein is at least 75%, at least 80%, at least 85%, at least 90%, at least 95%, at least 98%, or at least 99% identical to the amino acid sequence of SEQ ID NO: 218 and comprises an amino acid substitution of E567A. In some embodiments, the effector protein comprises an amino acid substitution of E567Q. In some embodiments, the effector protein is at least 75%, at least 80%, at least 85%, at least 90%, at least 95%, at least 98%, or at least 99% identical to the amino acid sequence of SEQ ID NO: 219. In some embodiments, the effector protein is at least 75%, at least 80%, at least 85%, at least 90%, at least 95%, at least 98%, or at least 99% identical to the amino acid sequence of SEQ ID NO: 219 and comprises an amino acid substitution of E567Q. In some embodiments, the effector protein comprises an amino acid substitution of D658A. In some embodiments, the effector protein is at least 75%, at least 80%, at least 85%, at least 90%, at least 95%, at least 98%, or at least 99% identical to the amino acid sequence of SEQ ID NO: 220. In some embodiments, the effector protein is at least 75%, at least 80%, at least 85%, at least 90%, at least 95%, at least 98%, or at least 99% identical to the amino acid sequence of SEQ ID NO: 220 and comprises an amino acid substitution of D658A. In some embodiments, the effector protein comprises an amino acid substitution of D658N. In some embodiments, the effector protein is at least 75%, at least 80%, at least 85%, at least 90%, at least 95%, at least 98%, or at least 99% identical to the amino acid sequence of SEQ ID NO: 221. In some embodiments, the effector protein is at least 75%, at least 80%, at least 85%, at least 90%, at least 95%, at least 98%, or at least 99% identical to the amino acid sequence of SEQ ID NO: 221 and comprises an amino acid substitution of D658N. In some embodiments, the effector protein comprises an amino acid sequence that is at least 75%, at least 80%, at least 85%, at least 90%, at least 95%, at least 99% or 100% identical to SEQ ID NO: 6 and comprises at least one amino acid substitution selected from D369A, D369N, E567A, E567Q, D658A, and D658N. In some embodiments, the effector protein comprises an amino acid sequence that is at least 75%, at least 80%, at least 85%, at least 90%, at least 95%, at least 99% or 100% identical to SEQ ID NO: 6 and comprises at least two amino acid substitution selected from D369A, D369N, E567A, E567Q, D658A, and D658N. In some embodiments, the effector protein comprises an amino acid sequence that is at least 75%, at least 80%, at least 85%, at least 90%, at least 95%, at least 99% or 100% identical to SEQ ID NO: 6 and comprises at least three amino acid substitution selected from D369A, D369N, E567A, E567Q, D658A, and D658N.

In some embodiments, the effector protein comprises an amino acid substitution of D267A. In some embodiments, the effector protein is at least 75%, at least 80%, at least 85%, at least 90%, at least 95%, at least 98%, or at least 99% identical to the amino acid sequence of SEQ ID NO: 222. In some embodiments, the effector protein is at least 75%, at least 80%, at least 85%, at least 90%, at least 95%, at least 98%, or at least 99% identical to the amino acid sequence of SEQ ID NO: 222 and comprises an amino acid substitution of D267A. In some embodiments, the effector protein comprises an amino acid sequence that is at least 75%, at least 80%, at least 85%, at least 90%, at least 95%, at least 99% or 100% identical to SEQ ID NO: 178 and comprises at least one amino acid substitution wherein one amino acid substitution is of D267A.

In some embodiments, the effector protein comprises an amino acid substitution of D267A. In some embodiments, the effector protein is at least 75%, at least 80%, at least 85%, at least 90%, at least 95%, at least 98%, or at least 99% identical to the amino acid sequence of SEQ ID NO: 223. In some embodiments, the effector protein is at least 75%, at least 80%, at least 85%, at least 90%, at least 95%, at least 98%, or at least 99% identical to the amino acid sequence of SEQ ID NO: 223 and comprises an amino acid substitution of D267A. In some embodiments, the effector protein comprises an amino acid substitution of D267N. In some embodiments, the effector protein is at least 75%, at least 80%, at least 85%, at least 90%, at least 95%, at least 98%, or at least 99% identical to the amino acid sequence of SEQ ID NO: 224. In some embodiments, the effector protein is at least 75%, at least 80%, at least 85%, at least 90%, at least 95%, at least 98%, or at least 99% identical to the amino acid sequence of SEQ ID NO: 224 and comprises an amino acid substitution of D267N. In some embodiments, the effector protein comprises an amino acid substitution of E363Q. In some embodiments, the effector protein is at least 75%, at least 80%, at least 85%, at least 90%, at least 95%, at least 98%, or at least 99% identical to the amino acid sequence of SEQ ID NO: 225. In some embodiments, the effector protein is at least 75%, at least 80%, at least 85%, at least 90%, at least 95%, at least 98%, or at least 99% identical to the amino acid sequence of SEQ ID NO: 225 and comprises an amino acid substitution of E363Q. In some embodiments, the effector protein comprises an amino acid sequence that is at least 75%, at least 80%, at least 85%, at least 90%, at least 95%, at least 99% or 100% identical to SEQ ID NO: 176 and comprises at least one amino acid substitution selected from D267A, D267N, and E363Q. In some embodiments, the effector protein comprises an amino acid sequence that is at least 75%, at least 80%, at least 85%, at least 90%, at least 95%, at least 99% or 100% identical to SEQ ID NO: 176 and comprises at least two amino acid substitution selected from D267A, D267N, and E363Q.

In some embodiments, the effector protein comprises an amino acid substitution of D326A. In some embodiments, the effector protein is at least 75%, at least 80%, at least 85%, at least 90%, at least 95%, at least 98%, or at least 99% identical to the amino acid sequence of SEQ ID NO: 226. In some embodiments, the effector protein is at least 75%, at least 80%, at least 85%, at least 90%, at least 95%, at least 98%, or at least 99% identical to the amino acid sequence of SEQ ID NO: 226 and comprises an amino acid substitution of D326A.

In some embodiments, the fusion partner protein comprises an amino acid sequence that is at least 75%, at least 80%, at least 85%, at least 90%, at least 95%, at least 98%, at least 99%, or 100% identical to SEQ ID NO: 400.

In another aspect, provided herein is a pharmaceutical composition comprising any one of the compositions disclosed herein, and a pharmaceutically acceptable carrier or diluent.

In another aspect, provided herein is a composition comprising at least one nucleic acid vector encoding any one of the fusion effector proteins described herein. Such a composition includes wherein the nucleic acid vector is a viral vector. Such a viral vector includes, in some embodiments, a AAV vector. In some embodiments, the composition comprises an adeno associated virus (AAV), wherein the AAV vector is packaged in the AAV. In some embodiments, the composition comprises at least one guide nucleic acid. In some embodiment, the viral vector of the composition encodes at least one guide nucleic acid.

In another aspect, provided herein is a method of modifying a target nucleic acid or the expression thereof. Such a method, in some embodiments, comprises contacting the target nucleic acid with any one of the compositions described herein, thereby modifying the target nucleic acid or the expression thereof. In some embodiments, the target nucleic acid is in a cell. In some embodiments, the cell is in vitro, ex vivo, or in vivo. In some embodiments, the cell is a prokaryotic cell. In some embodiments, the cell is a eukaryotic cell. In some embodiments, the cell is a mammalian cell. In some embodiments, the cell is an immune cell, such as a T cell.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features of the present disclosure are set forth with particularity in the appended claims. A better understanding of the features and advantages of the present disclosure can be obtained by reference to the following detailed description that sets forth illustrative embodiments, in which the principles of the disclosure are utilized, and the accompanying drawings of which:

FIG. 1 is a graphical representation of various fusion protein design constructs with dCasΦ.12 effector proteins and deaminase variants.

FIGS. 2A-2C illustrates observed percent base editing (BE) of A to G with dCasΦ.12-ABE8e, dCasΦ.12-ABE8e-TadA, and dCasΦ.12-TadA-ABE8e fusion protein variants (SEQ ID NOS: 530, 543-559). FIG. 2A illustrates binned maximum percent base editing data (A to G) of 18 combinatorial variants prepared with 4 optimized gRNA target sequences. “+++” indicates >7% maximum observed base editing (A to G), “++” indicates 4%-7% maximum observed base editing (A to G), and “+” indicates <4% maximum observed base editing (A to G). FIG. 2B illustrates up to 10.14% observed base editing of A to G with dCasΦ.12(E567Q)-ABE8e fusion protein (SEQ ID NO: 545) using gRNA PDCD1-target 87 (SEQ ID NO: 781). FIG. 2C illustrates up to 8.8% observed base editing of A to G with dCasΦ.12(E567Q)-ABE8e fusion protein (SEQ ID NO: 545) using gRNA PDCD1-target 75 (SEQ ID NO: 782).

FIGS. 3A-3B illustrates the effect of different dCasΦ.12 fusion protein variants (SEQ ID NOS: 530, 543-559) on base editing efficacy of fusion proteins categorized by effector protein catalytic mutation. FIG. 3A shows the maximum observed base editing of the various catalytic variants normalized to the maximum observed base editing of fusion protein dCasΦ.12(D369A)-ABE8e (SEQ ID NO: 530). FIG. 3B illustrates the maximum observed base editing at four different target sites (FUT8-Target 2, B2M-Target 2, PDCD1-Target 87, and PDCD1-Target 75) for various dCasΦ.12 fusion protein variants (SEQ ID NOS: 530, 543-559) normalized to the maximum observed base editing of dCasΦ.12(D369A)-ABE8e (SEQ ID NO: 530). “+++” indicates >2 (normalized value) maximum observed base editing (A to G), “++” indicates >1-2 (normalized value) maximum observed base editing (A to G), “+” indicates 1 (normalized value) maximum observed base editing (A to G) and “−” indicates <1 (normalized value) maximum observed base editing (A to G).

FIGS. 4A-4B illustrates the effect of effector protein design on fusion protein base editing function for different dCasΦ.12 fusion protein variants (SEQ ID NOS: 530, 543-559). Maximum observed base editing was normalized to the deaminase monomer, ABE8e (SEQ ID NO: 400), and TadA dimers were compared. TadA fused at the amino terminus (TadA-ABE8e) demonstrated similar and slightly worse base editing efficacy across the different catalytic mutant fusion proteins tested. FIG. 4B illustrates maximum observed base editing at four different target sites (FUT8-Target 2, B2M-Target 2, PDCD1-Target 87, and PDCD1-Target 75) for various dCasΦ.12 fusion protein variants (SEQ ID NOS: 530, 543-559) normalized to the maximum observed base editing of ABE8e base editors. “+++” indicates >2 (normalized value) maximum observed base editing (A to G), “++” indicates >1-2 (normalized value) maximum observed base editing (A to G), “+” indicates 1 (normalized value) maximum observed base editing (A to G) and “−” indicates <1 (normalized value) maximum observed base editing (A to G).

FIGS. 5A-5E illustrates indel occurrence in each dCasΦ.12 fusion protein variant (SEQ ID NOS: 530, 543-559) utilized for base editing. FIG. 5A illustrates indel occurrence for fusion proteins with base editor gRNA SEQ ID NO: 778. FIG. 5B illustrates indel occurrence for fusion proteins with base editor gRNA SEQ ID NO: 776. FIG. 5C illustrates indel occurrence for fusion proteins with base editor gRNA SEQ ID NO: 781. FIG. 5D illustrates indel occurrence for fusion proteins with base editor gRNA SEQ ID NO: 782. FIG. 5E illustrates that indel occurrence was observed at or near the effector protein cleavage site and no indel occurrence was observed at or near adenines within the base editing the base editing window.

FIGS. 6A-6D illustrates guide RNA design optimization for base editing using dCasΦ.12(E567Q)-XTEN10-ABE8e (SEQ ID NO: 545) in HEK293T cells. FIG. 6A illustrates base editing level percentage for each repeat: spacer combination for FUT8-target 2. FIG. 6B illustrates base editing level percentage for each repeat: spacer combination for B2M-target 2. FIG. 6C illustrates base editing level percentage for each repeat: spacer combination for PDCD1-target 87. FIG. 6D illustrates base editing level percentage for each repeat: spacer combination for PDCD1-target 75.

FIGS. 7A-7D illustrate percent maximum observed base editing in optimized gRNA repeat:spacer compositions using dCasΦ.12(E567Q)-XTEN10-ABE8e (SEQ ID NO: 545). FIG. 7A illustrates up to 15.2% observed base editing of A to G with dCasΦ.12(E567Q)-ABE8e fusion protein (SEQ ID NO: 545) using gRNA design PDCD1-target 87 (36:18) (gRNA SEQ ID NO: 783 combined with SEQ ID NO: 801). FIG. 7B illustrates up to 17.52% observed base editing of A to G with dCasΦ.12(E567Q)-ABE8e fusion protein (SEQ ID NO: 545) using gRNA design PDCD1-target 87 (20:20) (gRNA SEQ ID NO: 784 combined with SEQ ID NO: 802). FIG. 7C illustrates up to 12.24% observed base editing of A to G with dCasΦ.12(E567Q)-ABE8e fusion protein (SEQ ID NO: 545) using gRNA design FUT8-target 2 (36:18) (gRNA SEQ ID NO: 783 combined with SEQ ID NO: 789). FIG. 7D illustrates up to 14.12% observed base editing of A to G with dCasΦ.12(E567Q)-ABE8e fusion protein (SEQ ID NO: 545) using gRNA design FUT8-target 2 (20:18) (gRNA SEQ ID NO: 784 combined with SEQ ID NO: 789).

FIGS. 8A-8E illustrate change in gene expression of NEUROD1, HBG1, ASCL1, and LIN28A by different VPR-CasM fusions. FIG. 8A shows the change in gene expression by CasM.286251 (D267A) with an N terminal VPR fused by an XTEN10 linker. FIG. 8B shows the change in gene expression by CasM.19952 (D267A) with an N terminal VPR fused by an XTEN10 linker. FIG. 8C shows the change in gene expression by CasM.19952 (D267N) with an N terminal VPR fused by an XTEN10 linker. FIG. 8D shows the change in gene expression by CasM.19952 (E363Q) with an N terminal VPR fused by an XTEN10 linker. FIG. 8E shows the change in gene expression by CasM.124070 (D326A) with an N terminal VPR fused by an XTEN10 linker. The Y-axis shows the relative fold change of RNA levels. The X-axis shows the guide sequences tested. NT denotes a guide with the enzyme's repeat, but a scramble sequence spacer, gpool8 is a pooled control the guides, and dCas9 is a catalytically inactive “dead” Cas9.

### DETAILED DESCRIPTION

It is to be understood that both the foregoing general description and the following detailed description are exemplary and explanatory only and are not restrictive of the disclosure. Herein, the use of the singular includes the plural unless specifically stated otherwise. As used herein, the use of“or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

### Definitions

Unless otherwise indicated, all technical terms used herein have the same meaning as commonly understood by one of ordinary skill in the art to which this invention belongs. Unless otherwise indicated or obvious from context, the following terms have the following meanings:

As used in this specification and the appended claims, the singular forms “a,” “an,” and “the” include plural references unless the context clearly dictates otherwise. Any reference to “or” herein is intended to encompass “and/or” unless otherwise stated. As used herein, the term “and/or” includes any and all combinations of one or more of the associated listed items.

As used herein, the term “about” in reference to a number or range of numbers is understood to mean the stated number and numbers +/−10% thereof, or 10% below the lower listed limit and 10% above the higher listed limit for the values listed for a range.

As used herein, the term “comprise” and its grammatical equivalents specifies the presence of stated features, integers, steps, operations, elements, and/or components, but do not preclude the presence or addition of one or more other features, integers, steps, operations, elements, components, and/or groups thereof.

As used herein, the term “fusion effector protein,” also referred to simply as a “fusion protein,” refers to a protein comprising (i) an effector protein or a portion thereof that interacts with a guide nucleic acid, and (ii) a fusion partner protein. The effector protein of a fusion protein may be modified relative to a wildtype effector protein to reduce or abolish a catalytic activity of the effector protein. The catalytic activity is often nuclease activity. The resulting modified effector protein may be referred to as a catalytically inactive effector protein. Fusion effector proteins may modify a target nucleic acid sequence and/or target nucleic acid expression transiently or permanently.

As used herein, the term “base editing enzyme” refers to a protein, polypeptide or fragment thereof that is capable of catalyzing the chemical modification of a nucleobase of a deoxyribonucleotide or a ribonucleotide. Such a base editing enzyme, for example, is capable of catalyzing a reaction that modifies a nucleobase that is present in a nucleic acid molecule, such as DNA or RNA (single stranded or double stranded). Non-limiting examples of the type of modification that a base editing enzyme is capable of catalyzing includes converting an existing nucleobase to a different nucleobase, such as converting a cytosine to a guanine or thymine or converting an adenine to a guanine, hydrolytic deamination of an adenine or adenosine, or methylation of cytosine (e.g., CpG, CpA, CpT or CpC). A base editing enzyme itself may or may not bind to the nucleic acid molecule containing the nucleobase.

As used herein, the term “base editor” refers to a fusion protein comprising a base editing enzyme fused to an effector protein. The base editor is functional when the effector protein is coupled to a guide nucleic acid. The guide nucleic acid imparts sequence specific activity to the base editor. By way of non-limiting example, the effector protein may comprise a catalytically inactive Cas protein. Also, by way of non-limiting example, the base editing enzyme may comprise deaminase activity. Additional base editors are described herein.

As used herein, the term “effector protein” refers to a protein that is capable of modifying a nucleic acid molecule (e.g., by cleavage, deamination, recombination). Modifying the nucleic acid may modulate the expression of the nucleic acid molecule (e.g., increasing or decreasing the expression of a nucleic acid molecule). Modifying the nucleic acid may result in modifying the expression or activity of a translation product of the nucleic acid. The effector protein may be a CRISPR associated (Cas) protein.

As used herein, a “catalytically inactive effector protein” refers to an effector protein that is modified relative to a naturally-occurring effector protein to have a reduced or eliminated catalytic activity relative to that of the naturally-occurring effector protein, but retains its ability to interact with a guide nucleic acid. The catalytic activity that is reduced or eliminated is often a nuclease activity. The naturally-occurring effector protein may be a wildtype protein. In some embodiments, the catalytically inactive effector protein is referred to as a catalytically inactive variant of an effector protein, e.g., a Cas effector protein.

As used herein, the terms “fusion partner protein” or “fusion partner” refers to a protein, polypeptide or peptide that is fused to an effector protein. The fusion partner generally imparts some function to the fusion protein that is not provided by the effector protein. By way of non-limiting example, the fusion partner may provide a detectable signal. By way of non-limiting example, the fusion partner may modify a target nucleic acid, including changing a nucleobase of the target nucleic acid and making a chemical modification to one or more nucleotides of the target nucleic acid. By way of non-limiting example, the fusion partner may be capable of modulating the expression of a target nucleic acid. By way of non-limiting example, the fusion partner may inhibit, reduce, activate or increase expression of a target nucleic acid via additional proteins or nucleic acid modifications to the target sequence. By way of non-limiting example, the fusion partner may make an epigenetic modification of that target nucleic acid.

As used herein, the term “functional fragment” refers to a fragment of a protein that retains some function relative to the entire protein. Non-limiting examples of functions are nucleic acid binding, protein binding, nuclease activity, nickase activity, deaminase activity, demethylase activity, or acetylation activity.

As used herein, the term “cleavage assay” refers to a programmable nuclease cleavage assay wherein effector proteins are tested for their ability to cleave a nucleic acid. The nucleic acid may be single stranded or double stranded. The nucleic acid may be a single strand of a double stranded nucleic acid. The cleavage assay may test for cis cleavage (double stranded break). The cleavage assay may test for trans cleavage, also referred to as transcollateral cleavage (e.g., cleavage of a nucleic acid that is near, but not hybridized to the guide nucleic acid).

As used herein, the terms “individual,” “subject,” and “patient” are used interchangeably and include any member of the animal kingdom, including humans.

As used herein, the term, “% identical,” refers to the extent to which two sequences (nucleotide or amino acid) have the same residue at the same positions in an alignment. For example, the phrase, “the amino acid sequence of the effector protein is X % identical to SEQ ID NO: Y” refers to the percent of the amino acids in the effector protein that are identical to the corresponding residues of SEQ ID NO: Y when the amino acid sequence of the effector protein is aligned with SEQ ID NO: Y for maximum identity. Generally, computer programs are employed for such calculations. Illustrative programs that compare and align pairs of sequences, include ALIGN (Myers and Miller, Comput Appl Biosci. 1988 March; 4(1):11-7); FASTA (Pearson and Lipman, Proc Natl Acad Sci USA. (1988) April; 85(8):2444-8; Pearson, Methods Enzymol. (1990) 183:63-98); and gapped BLAST (Altschul et al., Nucleic Acids Res. (1997) September 1; 25(17):3389-40), BLASTP, BLASTN, or GCG (Devereux et al., Nucleic Acids Res. (1984) January 11; 12(1 Pt 1):387-95).

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “prime editing enzyme” refers to a protein, polypeptide or fragment thereof that is capable of catalyzing the modification (insertion, deletion, or base-to-base conversion) of a target nucleotide or nucleotide sequence in a nucleic acid. A prime editing enzyme capable of catalyzing such a reaction includes a reverse transcriptase. A prime editing enzyme may require a prime editing guide RNA (pegRNA) to catalyze the modification. Such a pegRNA can be capable of identifying the nucleotide or nucleotide sequence in the target nucleic acid to be edited and encoding the new genetic information that replaces the targeted nucleotide or nucleotide sequence in the nucleic acid. A prime editing enzyme may require a prime editing guide RNA (pegRNA) and a single guide RNA to catalyze the modification.

As used herein, the term “transcriptional activator” refers to a protein, polypeptide or fragment thereof that is capable of activating or increasing expression of a target nucleic acid by promoting transcription. A transcriptional activator can activate or increase expression of a target nucleic acid by, for example, promoting transcription by any number of mechanisms, including, recruitment of other transcription factor proteins, modification of target DNA (e.g., demethylation), recruitment of a DNA modifier, modulation of histones associated with target DNA, recruitment of a histone modifier (e.g., acetylation and/or methylation of histones), or a combination thereof.

As used herein, the term “transcriptional inhibitor” refers to a protein, polypeptide or fragment thereof that is capable of deactivating or decreasing expression of a target nucleic acid by preventing transcription. A transcriptional inhibitor can deactivate or decrease expression of a target nucleic acid by, for example, preventing transcription by any number of mechanisms, including, recruitment of transcriptional repressors, modification of target DNA (e.g., methylation), recruitment of a DNA modifier, modulation of histones associated with target DNA, recruitment of a histone modifier (e.g., deacetylation and/or methylation of histones), or a combination thereof.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

Disclosed herein are non-naturally occurring compositions and systems comprising at least one of a fusion effector protein (e.g., an effector protein or a portion thereof that interacts with a guide nucleic acid, and a fusion partner protein) and an engineered guide nucleic acid, which may simply be referred to herein as a fusion effector protein and a guide nucleic acid, respectively. In general, a fusion effector protein and a guide nucleic acid refer to a fusion effector protein and a guide nucleic acid, respectively, that are not found in nature. In some embodiments, systems and compositions herein comprise at least one non-naturally occurring component. For example, compositions and systems may comprise a guide nucleic acid, wherein the sequence of the guide nucleic acid is different or modified from that of a naturally-occurring guide nucleic acid. In some embodiments, compositions and systems comprise at least two components that do not naturally occur together. For example, compositions and systems may comprise a guide nucleic acid comprising a repeat region and a spacer region which do not naturally occur together. Also, by way of example, composition and systems may comprise a guide nucleic acid and a fusion effector protein having an effector protein that do not naturally occur together. Conversely, and for clarity, an effector protein or guide nucleic acid that is “natural,” “naturally-occurring,” or “found in nature” includes effector proteins and guide nucleic acids from cells or organisms that have not been genetically modified by a human or machine.

In some embodiments, the guide nucleic acid comprises a non-natural nucleobase sequence. In some embodiments, the non-natural sequence is a nucleobase sequence that is not found in nature. The non-natural sequence may comprise a portion of a naturally-occurring sequence, wherein the portion of the naturally-occurring sequence is not present in nature absent the remainder of the naturally-occurring sequence. In some embodiments, the guide nucleic acid comprises two naturally-occurring sequences arranged in an order or proximity that is not observed in nature. In some embodiments, compositions and systems comprise a ribonucleotide complex comprising an effector protein and a guide nucleic acid that do not occur together in nature. Engineered guide nucleic acids may comprise a first sequence and a second sequence that do not occur naturally together. For example, a guide nucleic acid may comprise a sequence of a naturally-occurring repeat region and a spacer region that is complementary to a naturally-occurring eukaryotic sequence. The guide nucleic acid may comprise a sequence of a repeat region that occurs naturally in an organism and a spacer region that does not occur naturally in that organism. A guide nucleic acid may comprise a first sequence that occurs in a first organism and a second sequence that occurs in a second organism, wherein the first organism and the second organism are different. The guide nucleic acid may comprise a third sequence disposed at a 3′ or 5′ end of the guide nucleic acid, or between the first and second sequences of the guide nucleic acid. For example, a guide nucleic acid may comprise a naturally occurring crRNA and tracrRNA coupled by a linker sequence.

In some embodiments, compositions and systems described herein comprise a fusion effector protein having an effector protein that is similar to a naturally occurring effector protein. The effector protein may lack a portion of the naturally occurring effector protein. The effector protein may comprise a mutation relative to the naturally-occurring effector protein, wherein the mutation is not found in nature. The effector protein may also comprise at least one additional amino acid relative to the naturally-occurring effector protein. For example, the effector protein may comprise an addition of a nuclear localization signal relative to the natural occurring effector protein. In certain embodiments, the nucleotide sequence encoding the effector protein is codon optimized (e.g., for expression in a eukaryotic cell) relative to the naturally occurring sequence.

### I. Fusion Effector Proteins

Disclosed herein, in some aspects, are compositions comprising a fusion effector protein and uses thereof. In general, fusion effector proteins comprise an effector protein (e.g., a Cas protein), and a fusion partner protein (also referred to simply as a fusion partner) that is heterologous to the Cas protein. Fusion partner proteins include, but are not limited to non Cas enzymes such as polymerases, acetyltransferases, methyltransferases, deaminases, exonucleases, proteases, kinases, etc. In general, the fusion partner is fused or linked to the effector protein. In some embodiments, the amino terminus of the fusion partner is linked/fused to the carboxy terminus of the effector protein. In some embodiments, the carboxy terminus of the fusion partner protein is linked/fused to the amino terminus of the effector protein by the linker. Exemplary effector proteins are provided in TABLE 1 and exemplary fusion partners are provided in TABLE 2. Exemplary fusion proteins are provided in TABLE 6.

In some embodiments, the fusion partner is not an effector protein as described herein. In some embodiments, the fusion partner comprises a second effector protein or a multimeric form thereof. Accordingly, in some embodiments, the fusion protein comprises more than one effector protein. In such embodiments, the fusion protein can comprise at least two effector proteins that are same. In some embodiments, the fusion protein comprises at least two effector proteins that are different. In some embodiments, the multimeric form is a homomeric form. In some embodiments, the multimeric form is a heteromeric form. Unless otherwise indicated, reference to effector proteins throughout the present disclosure include fusion proteins comprising the effector protein described herein and a fusion partner.

In general, fusion effector proteins comprise an effector protein or a portion thereof, and a fusion partner protein. In some embodiments, compositions and systems that comprise a fusion effector protein further comprises a guide nucleic acid, wherein at least a portion of the guide nucleic acid hybridizes to a target nucleic acid, and the fusion partner modulates the target nucleic acid or expression thereof.

In some embodiments, fusion effector proteins modify a target nucleic acid or the expression thereof. In some embodiments, the modifications are transient (e.g., transcription repression or activation). In some embodiments, the modifications are inheritable. For instance, epigenetic modifications made to a target nucleic acid, or to proteins associated with the target nucleic acid, e.g., nucleosomal histones, in a cell, are observed in cells produced by proliferation of the cell.

In some embodiments, fusion effector proteins modify a target nucleic acid or the expression thereof, wherein the target nucleic acid comprises a deoxyribonucleoside, a ribonucleoside or a combination thereof. The target nucleic acid may comprise or consist of a single stranded RNA (ssRNA), a double-stranded RNA (dsRNA), a single-stranded DNA (ssDNA), or a double stranded DNA (dsDNA). Non-limiting examples of fusion partners for modifying ssRNA include, but are not limited to, splicing factors (e.g., RS domains); protein translation components (e.g., translation initiation, elongation, and/or release factors; e.g., eIF4G); RNA methylases; RNA editing enzymes (e.g., RNA deaminases, e.g., adenosine deaminase acting on RNA (ADAR), including A to I and/or C to U editing enzymes); helicases; and RNA-binding proteins.

In some embodiments, a fusion partner is directly or indirectly linked to an effector protein via a linker. In some embodiments, the linker comprises an amide bond, a peptide bond, an amino acid, a peptide, a nucleotide, a polymer, or a combination thereof. In some embodiments, the fusion partner comprises a plurality of fusion partner. In some embodiments, the plurality of fusion partner comprises at least two, at least three, at least four, at least five, at least six, at least seven, at least eight, at least nine, or at least ten fusion partners. In some embodiments, the fusion protein comprises two fusion partners.

### Effector Proteins

Disclosed herein are fusion effector proteins and uses thereof, wherein the fusion effector proteins comprise an effector protein. In some embodiments, the effector protein is a Cas effector protein. In some embodiments, the effector protein is a Cas protein within the Class 2 type CRISPR-Cas classification, which includes Type II, V and VI Cas proteins. In some embodiments, the effector protein is a Type II Cas effector protein. In some embodiments the effector protein is a Type IIS restriction endonuclease as described in WO2021084533, which is hereby incorporated by reference in its entirety. In some embodiments, the effector protein is a Cas9 effector protein. In some embodiments, the effector protein comprises a functional domain of a Cas9 effector protein (e.g., an HNH domain or RuvC domain). In some embodiments, the effector protein comprises a dead Cas9 (dCas9) or a Cas9 nickase (nCas9). Effector proteins with nickase activity is further described in WO2020223634, which is hereby incorporated by reference in its entirety. In some embodiments, the effector protein comprises a modified Staphylococcus aureus Cas9 (SaCas9), Streptococcus thermophilus 1 Cas9 (St1Cas9), a modified Streptococcus pyogenes Cas9 (SpCas9). In some embodiments, the effector protein comprises a variant of SpCas9 having an altered protospacer-adjacent motif (PAM) specificity. In some embodiments, the altered PAM has specificity for the nucleic acid sequence 5′-NGC-3′.

In some embodiments, the effector protein is a Type V Cas effector protein. Whereas Type II Cas effector proteins generally comprise two types of nuclease domains (HNH and RuvC), Type V Cas effector proteins are generally characterized by a single type of nuclease domain (RuvC), and are compact (e.g., less than about 1200 amino acids in length). Moreover, Type V Cas effector proteins (e.g., Cas12 or Cas14) lack an HNH domain as described in WO2020028729, which is hereby incorporated by reference in its entirety. A Cas12 nuclease as described herein can generally cleave a nucleic acid via a single catalytic RuvC domain. The RuvC domain is within a nuclease, or “NUC” lobe of the protein, and the Cas12 nucleases further comprise a recognition, or “REC” lobe. The REC and NUC lobes are connected by a bridge helix and the Cas12 proteins additionally include two domains for PAM recognition termed the PAM interacting (PI) domain and the wedge (WED) domain (Murugan et al., Mol Cell. 2017 Oct. 5; 68(1): 15-25). A programmable Cas12 nuclease can be a Cas12a (also referred to as Cpf1) protein, a Cas12b protein, Cas12c protein, Cas12d protein, or a Cas12e protein. For further examples of Type V Cas effector proteins, see WO2020028729, which is hereby incorporated by reference in its entirety. In some embodiments, the nuclease comprises a RuvC-I subdomain, a RuvC-II subdomain, and a RuvC-III subdomain (see WO2020142754, which is hereby incorporated by reference in its entirety, for further information regarding Type V programmable nucleases, related compositions, and methods of use). In some embodiments, the Type V Cas protein is a Cas14 protein. In some embodiments, the Cas14 protein is selected from Cas14a and Cas14b. In some embodiments, the Cas14 protein is Cas14a.1 (SEQ ID NO: 8).

In some embodiments, the Type V Cas effector protein is less than about 1200, less than about 1100, less than about 1000, less than about 900, less than about 800, less than about 700, less than about 600, less than about 500, or less than about 400 amino acids in length, but greater than about 300 amino acids in length. In some embodiments, the effector protein is a Cas12a/Cpf1 protein. In some embodiments, the effector protein is a Cas12b/C2c1 protein. In some embodiments, the effector protein is a Cas12c/C2c3 protein. In some embodiments, the effector protein is a Cas12d/CasY protein. In some embodiments, the effector protein is a Cas12e/CasX protein. In some embodiments, the effector protein is a Cas12g protein. In some embodiments, the effector protein is a Cas12h protein. In some embodiments, the effector protein is a Cas12i protein. In some embodiments, the effector protein is a Cas12j/Cas0 protein. In some embodiments, the effector protein is a Cas12j protein. In some embodiments, the effector protein is a Cas12j protein and may be referred to as a CasΦ protein. In some embodiments, the Casφprotein is selected from the group consisting of: Casφ.12 (SEQ ID NO: 6-7 and 217-221), Casφ.18 (SEQ ID NO: 28), Casφ.32 (SEQ ID NO: 42), Casφ.20 (SEQ ID NO: 30), Casφ.28 (SEQ ID NO: 38), and Casφ.45 (SEQ ID NO: 54). In some embodiments, the effector protein is a catalytically inactive or “dead” Cas12j (e.g., dCasΦ. In some embodiments, the effector protein is a catalytically inactive effector protein. In some embodiments, the effector protein is a dCasΦ.12 protein. In some embodiments, dCasΦ.12 protein comprises any one of amino acid sequences of SEQ ID NO: 7 and 217-221.

A catalytically inactive effector protein may be generated by changing an amino acid that confers a catalytic activity (also referred to as a “catalytic residue”) to a different amino acid that does not support the catalytic activity. In some embodiments, the different amino acid has an aliphatic side chain. In some embodiments, the different amino acid is glycine. In some embodiments, the different amino acid is valine. In some embodiments, the different amino acid is leucine. In some embodiments, the different amino acid is alanine. In some embodiments, the amino acid is aspartate and it is substituted with asparagine. In some embodiments, the amino acid is glutamate and it is substituted with glutamine. An amino acid that confers catalytic activity may be identified by performing sequence alignment of an unmodified effector protein with a similar enzyme having at least one identified catalytic residue; selecting at least one putative catalytic residue in the unmodified effector protein within the portion of the unmodified effector protein that aligns with a portion of the similar enzyme that comprises the identified catalytic residue; substituting the at least one putative catalytic residue of the unmodified effector protein with the different amino acid; and comparing the catalytic activity of the unmodified effector protein to the modified effector protein. A similar enzyme may be an enzyme that is at least 50%, at least 60%, at least 70%, at least 80%, or at least 90% identical to the unmodified effector protein. A similar enzyme may be an enzyme that is not greater than 99.9% identical to the unmodified effector protein. In some embodiments, the similar enzyme is a Type V Cas effector. In some embodiments, the similar enzyme comprises a RuvC domain. In some embodiments, the portion of the unmodified effector protein that aligns with a portion of the similar enzyme is at least 10 amino acids, at least 20 amino acids, at least 30 amino acids, at least 40 amino acids, at least 50 amino acids, at least 60 amino acids, at least 70 amino acids, at least 80 amino acids, at least 90 amino acids, or at least 100 amino acids in length. In some embodiments, the portion of the unmodified effector protein that aligns with a portion of the similar enzyme is not greater than 200 amino acids. In some embodiments, the portion of the unmodified effector protein that aligns with a portion of the similar enzyme comprises a RuvC domain. In some embodiments, comparing the catalytic activity comprises performing a cleavage assay. An example of generating a catalytically inactive effector protein is provided in Example 4. For further information regarding methods of sequence specific cleavage of a nucleic acid, see WO2020142754, which is hereby incorporated by reference in its entirety.

In some aspects, the programmable nuclease is a programmable Type VI Cas effector protein also described in US 20210078002, which is hereby incorporated by reference in its entirety. In some aspects, the programmable Type VI Cas effector protein is a programmable Cas13 nuclease. In some aspects, the programmable Cas13 nuclease is Cas13a, Cas13b, Cas13c, Cas13d, or Cas13e. See US 2020/078002 for further description regarding Type VI Cas effector proteins.

In some embodiments, an effector protein comprises a functional domain of the effector protein. In some embodiments, the functional domain comprises a rCas9 domain. In some embodiments, the functional domain comprises a Cas13 domain. In some embodiments, the Cas13 domain is a HEPN domain. In some embodiments, the functional domain is selected from a PUS1 domain and a PUS7 domain.

In general, effector proteins provided herein comprise an effector protein or a portion thereof. In some embodiments, the effector protein comprises an amino acid sequence that is at least 75%, at least 80%, at least 85%, at least 90%, at least 95%, at least 98%, at least 99% or 100% identical to any one of SEQ ID NOs: 1-226. In some embodiments, the amino acid sequence of the effector protein is at least 75%, at least 80%, at least 85%, at least 90%, at least 95%, at least 98%, at least 99% or 100% identical to any one of SEQ ID NOs: 1-226. SEQ ID NOs: 1-226 are provided in TABLE 1 below.

In some embodiments, the amino acid sequence of the effector protein comprises at least 100, at least 110, at least 120, at last 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, or at least 500 contiguous amino acids that are at least 75%, at least 80%, at least 85%, at least 90%, at least 95%, at least 98%, at least 99% or 100% identical to any one of SEQ ID NOs: 1-226.

In some embodiments, the length of the effector protein comprises at least 100, at least 110, at least 120, at last 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, or at least 500 contiguous amino acids that are at least 75%, at least 80%, at least 85%, at least 90%, at least 95%, at least 98%, at least 99% or 100% identical to any one of SEQ ID NOs: 1-226. In some embodiments, the length of the effector protein is less than about 900, less than about 800, less than about 700, less than about 600, or less than about 500 linked amino acids, or at least 350 linked amino acids that are at least 75%, at least 80%, at least 85%, at least 90%, at least 95%, at least 98%, at least 99% or 100% identical to any one of SEQ ID NOs: 1-226. In some embodiments, the length of the effector protein is at least 350 linked amino acids that is at least 75%, at least 80%, at least 85%, at least 90%, at least 95%, at least 98%, at least 99% or 100% identical to any one of SEQ ID NOs: 1-226.

In some embodiments, the amino acid sequence of the effector protein does not comprise more than about 500, does not comprise more than about 550, does not comprise more than about 600, does not comprise more than about 650, does not comprise more than about 700, does not comprise more than about 750, does not comprise more than about 800, does not comprise more than about 850, does not comprise more than about 900, does not comprise more than about 950, or does not comprise more than about 1000 contiguous amino acids that are more than 75%, more than 80%, more than 85%, more than 90%, more than 95%, more than 98%, more than 99%, or 100% identical to any one of SEQ ID NOs: 1-226.

In some embodiments, the amino acid sequence of the effector protein is a modified form of a sequence selected from TABLE 1. The modified form of the sequence may comprise at least one amino acid that differs from the amino acid at the corresponding position of the sequence selected from TABLE 1, wherein the presence of the amino acid that differs results in reduced nuclease activity of the effector protein, as measured by a cleavage assay. In some embodiments, the modified form of the sequence comprises two, three, four or more amino acids that differ from the amino acids at the corresponding positions of the sequence selected from TABLE 1. In some embodiments, the one or more amino acids that differ renders the effector protein catalytically inactive.

TABLE 1
Exemplary Effector Protein Sequences

| SEQ ID NO. | Nickname |
|---|---|
| 1 | Active SpyCas9 |
| 2 | Dead SpyCas9 |
| 3 | Dead PspCas13b |
| 4 | Nickase SpyCas9 (D10A) |
| 5 | Nickase SpyCas9 (H840A) |
| 6 | Active CasΦ.12 |
| 7 | Dead CasΦ.12 (D369A) |
| 8 | Cas14a.1 |
| 9 | Cas14a.280852 |
| 10 | Nickase SpyCas9 |
| 11 | Cas13 |
| 12 | CasΦ.1 |
| 13 | CasΦ.2 |
| 14 | CasΦ.3 |
| 15 | CasΦ.4 |
| 16 | CasΦ.5 |
| 17 | CasΦ.6 |
| 18 | CasΦ.7 |
| 19 | CasΦ.8 |
| 20 | CasΦ.9 |
| 21 | CasΦ.10 |
| 22 | CasΦ.11 |
| 23 | CasΦ.13 |
| 24 | CasΦ.14 |
| 25 | CasΦ.15 |
| 26 | CasΦ.16 |
| 27 | CasΦ.17 |
| 28 | CasΦ.18 |
| 29 | CasΦ.19 |
| 30 | CasΦ.20 |
| 31 | CasΦ.21 |
| 32 | CasΦ.22 |
| 33 | CasΦ.23 |
| 34 | CasΦ.24 |
| 35 | CasΦ.25 |
| 36 | CasΦ.26 |
| 37 | CasΦ.27 |
| 38 | CasΦ.28 |
| 39 | CasΦ.29 |
| 40 | CasΦ.30 |
| 41 | CasΦ.31 |
| 42 | CasΦ.32 |
| 43 | CasΦ.33 |
| 44 | CasΦ.34 |
| 45 | CasΦ.35 |
| 46 | CasΦ.36 |
| 47 | CasΦ.37 |
| 48 | CasΦ.38 |
| 49 | CasΦ.39 |
| 50 | CasΦ.41 |
| 51 | CasΦ.42 |
| 52 | CasΦ.43 |
| 53 | CasΦ.44 |
| 54 | CasΦ.45 |
| 55 | CasΦ.46 |
| 56 | CasΦ.47 |
| 57 | CasΦ.48 |
| 58 | CasΦ.49 |
| 59 | Cas14 ortholog 1 |
| 60 | Cas14 ortholog 3 |
| 61 | Cas14 ortholog 4 |
| 62 | Cas14 ortholog 5 |
| 63 | Cas14 ortholog 6 |
| 64 | Cas14 ortholog 7 |
| 65 | Cas14 ortholog 9 |
| 66 | Cas14 ortholog 10 |
| 67 | Cas14 ortholog 11 |
| 68 | Cas14 ortholog 12 |
| 69 | Cas14 ortholog 13 |
| 70 | Cas14 ortholog 14 |
| 71 | Cas14 ortholog 15 |
| 72 | Cas14 ortholog 16 |
| 73 | Cas14 ortholog 17 |
| 74 | Cas14 ortholog 18 |
| 75 | Cas14 ortholog 19 |
| 76 | Cas14 ortholog 20 |
| 77 | Cas14 ortholog 21 |
| 78 | Cas14 ortholog 22 |
| 79 | Cas14 ortholog 23 |
| 80 | Cas14 ortholog 24 |
| 81 | Cas14 ortholog 25 |
| 82 | Cas14 ortholog 26 |
| 83 | Cas14 ortholog 27 |
| 84 | Cas14 ortholog 28 |
| 85 | Cas14 ortholog 29 |
| 86 | Cas14 ortholog 30 |
| 87 | Cas14 ortholog 31 |
| 88 | Cas14 ortholog 32 |
| 89 | Cas14 ortholog 33 |
| 90 | Cas14 ortholog 34 |
| 91 | Cas14 ortholog 35 |
| 92 | Cas14 ortholog 36 |
| 93 | Cas14 ortholog 37 |
| 94 | Cas14 ortholog 38 |
| 95 | Cas14 ortholog 39 |
| 96 | Cas14 ortholog 40 |
| 97 | Cas14 ortholog 41 |
| 98 | Cas14 ortholog 42 |
| 99 | Cas14 ortholog 43 |
| 100 | Cas14 ortholog 44 |
| 101 | Cas14 ortholog 45 |
| 102 | Cas14 ortholog 46 |
| 103 | Cas14 ortholog 47 |
| 104 | Cas14 ortholog 48 |
| 105 | Cas14 ortholog 49 |
| 106 | Cas14 ortholog 50 |
| 107 | Cas14 ortholog 51 |
| 108 | Cas14 ortholog 52 |
| 109 | Cas14 ortholog 53 |
| 110 | Cas14 ortholog 54 |
| 111 | Cas14 ortholog 55 |
| 112 | Cas14 ortholog 56 |
| 113 | Cas14 ortholog 57 |
| 114 | Cas14 ortholog 58 |
| 115 | Cas14 ortholog 59 |
| 116 | Cas14 ortholog 60 |
| 117 | Cas14 ortholog 61 |
| 118 | Cas14 ortholog 62 |
| 119 | Cas14 ortholog 63 |
| 120 | Cas14 ortholog 64 |
| 121 | Cas14 ortholog 65 |
| 122 | Cas14 ortholog 66 |
| 123 | Cas14 ortholog 67 |
| 124 | Cas14 ortholog 68 |
| 125 | Cas14 ortholog 69 |
| 126 | Cas14 ortholog 70 |
| 127 | Cas14 ortholog 71 |
| 128 | Cas14 ortholog 72 |
| 129 | Cas14 ortholog 73 |
| 130 | Cas14 ortholog 74 |
| 131 | Cas14 ortholog 75 |
| 132 | Cas14 ortholog 76 |
| 133 | Cas14 ortholog 77 |
| 134 | Cas14 ortholog 78 |
| 135 | Cas14 ortholog 79 |
| 136 | Cas14 ortholog 80 |
| 137 | Cas14 ortholog 81 |
| 138 | Cas14 ortholog 82 |
| 139 | Cas14 ortholog 83 |
| 140 | Cas14 ortholog 84 |
| 141 | Cas14 ortholog 85 |
| 142 | Cas14 ortholog 86 |
| 143 | Cas14 ortholog 87 |
| 144 | Cas14 ortholog 88 |
| 145 | Cas14 ortholog 89 |
| 146 | Cas14 ortholog 90 |
| 147 | Cas14 ortholog 91 |
| 148 | Cas14 ortholog 92 |
| 149 | Cas14 ortholog 93 |
| 150 | Cas14 ortholog 94 |
| 151 | Cas14 ortholog 95 |
| 152 | Cas14 ortholog 96 |
| 153 | Cas14 ortholog 97 |
| 154 | CasM.298706 |
| 155 | CasM.280604 |
| 156 | CasM.281060 |
| 157 | CasM.284933 |
| 158 | CasM.287908 |
| 159 | CasM.288518 |
| 160 | CasM.293891 |
| 161 | CasM.294270 |
| 162 | CasM.294491 |
| 163 | CasM.295047 |
| 164 | CasM.299588 |
| 165 | CasM.277328 |
| 166 | CasM.297894 |
| 167 | CasM.291449 |
| 168 | CasM.297599 |
| 169 | CasM.286588 |
| 170 | CasM.286910 |
| 171 | CasM.292335 |
| 172 | CasM.293576 |
| 173 | CasM.294537 |
| 174 | CasM.298538 |
| 175 | CasM.19924 |
| 176 | CasM.19952 |
| 177 | CasM.274559 |
| 178 | CasM.286251 |
| 179 | CasM.288480 |
| 180 | CasM.288668 |
| 181 | CasM.289206 |
| 182 | CasM.290598 |
| 183 | CasM.290816 |
| 184 | CasM.295071 |
| 185 | CasM.295231 |
| 186 | CasM.292139 |
| 187 | CasM.279423 |
| 188 | CasM.20054 |
| 189 | CasM.282673 |
| 190 | CasM.282952 |
| 191 | CasM.283262 |
| 192 | CasM.284833 |
| 193 | CasM.287700 |
| 194 | CasM.291507 |
| 195 | CasM.293410 |
| 196 | CasM.295105 |
| 197 | CasM.295187 |
| 198 | CasM.295929 |
| 199 | Cas13 ortholog 1 |
| 200 | Cas13 ortholog 2 |
| 201 | Cas13 ortholog 3 |
| 202 | Cas13 ortholog 4 |
| 203 | Cas13 ortholog 5 |
| 204 | CasM.1584 |
| 205 | CasM.1730 |
| 206 | CasM.1770 |
| 207 | CasM.1816 |
| 208 | CasM.1862939 |
| 209 | CasM.1862895 |
| 210 | CasM.1862903 |
| 211 | CasM.1862909 |
| 212 | CasM.1862917 |
| 213 | CasM.1862921 |
| 214 | CasM.1862947 |
| 215 | CasM.1422 |
| 216 | CasM.1740 |
| 217 | Dead CasΦ.12 (D369N) |
| 218 | Dead CasΦ.12 (E567A) |
| 219 | Dead CasΦ.12 (E567Q) |
| 220 | Dead CasΦ.12 (D658A) |
| 221 | Dead CasΦ.12 (D658N) |
| 222 | CasM.286251 (D267A) |
| 223 | CasM.19952 (D267A) |
| 224 | CasM.19952 (D267N) |
| 225 | CasM.19952 (E363Q) |
| 226 | CasM.124070 (D326A) |

In some embodiments, the amino acid sequence of the effector protein is modified relative to a naturally-occurring effector protein. Such modified effector proteins may be referred to as an engineered effector protein. In some embodiments, the engineered effector protein has been modified to inactivate a catalytically active nuclease domain (e.g., a RuvC domain, HNH domain) of the naturally-occurring effector protein. In some embodiments, the engineered effector protein has been modified to reduce the activity of a catalytically active nuclease domain of the naturally-occurring effector protein. The engineered effector protein may have less than 90%, less than 80%, less than 70%, less than 60%, less than 50%, less than 40%, less than 30%, less than 20%, less than 10%, less than 5%, or less than 1% of the nucleic acid-cleaving activity as compared to the naturally-occurring effector protein as compared in a cleavage assay. In some embodiments, the effector protein has been modified to comprise at least 1, at least 2, at least 3, at least 4, or at least 5 amino acid modifications relative to the non-modified version (e.g., wild-type of naturally occurring version) of the effector protein. The amino acid modification(s) may comprise a deletion, insertion, or substitution of an amino acid.

In some embodiments, compositions, systems, and methods described herein comprise an effector protein or a nucleic acid encoding the effector protein, wherein the effector protein comprises one or more amino acid alterations relative to the sequence recited in TABLE 1. In some embodiments, the effector protein comprising one or more amino acid alterations is a variant of an effector protein described herein. It is understood that any reference to an effector protein herein also refers to an effector protein variant as described herein. In some embodiments, the one or more amino acid alterations comprises conservative substitutions, non-conservative substitutions, conservative deletions, non-conservative deletions, or combinations thereof. In some embodiments, an effector protein or a nucleic acid encoding the effector protein comprises 1 amino acid alteration, 2 amino acid alterations, 3 amino acid alterations, 4 amino acid alterations, 5 amino acid alterations, 6 amino acid alterations, 7 amino acid alterations, 8 amino acid alterations, 9 amino acid alterations, 10 amino acid alterations or more relative to the sequence recited in TABLE 1.

In some embodiments, 10% or less of the amino acids of the effector protein are substituted with conservative amino acid substitutions, and not more than 1, 2, 3, 4, 5, 10, 15, 20, 25, 30, 35 or 40 of the amino acids of the effector protein are substituted with non-conservative amino acid substitutions. In some embodiments, 10% or less of the amino acids of the effector protein are substituted with conservative amino acid substitutions, and not more than 1% of the amino acids of the effector protein are substituted with non-conservative amino acid substitutions. In some embodiments, 5% or less of the amino acids of the effector protein are substituted with conservative amino acid substitutions, and not more than 1% of the amino acids of the effector protein are substituted with non-conservative amino acid substitutions.

In some embodiments, compositions, systems and methods described herein comprise an effector protein or a nucleic acid encoding the effector protein, wherein the amino acid sequence of the effector protein comprises at least about 200 contiguous amino acids or more of the sequence recited in TABLE 1. In some embodiments, the amino acid sequence of an effector protein provided herein comprises at least about 200, at least about 220, at least about 240, at least about 260, at least about 280, at least about 300, at least about 320, at least about 340, at least about 360, at least about 380, at least about 400 contiguous amino acids, at least about 420 contiguous amino acids, at least about 440 contiguous amino acids, at least about 460 contiguous amino acids, at least about 480 contiguous amino acids, at least about 500 contiguous amino acids, at least about 520 contiguous amino acids, at least about 540 contiguous amino acids, at least about 560 contiguous amino acids, at least about 580 contiguous amino acids, at least about 600 contiguous amino acids, at least about 620 contiguous amino acids, at least about 640 contiguous amino acids, at least about 660 contiguous amino acids, at least about 680 contiguous amino acids, at least about 700 contiguous amino acids, or more of the sequence recited in TABLE 1.

In some embodiments, compositions, systems and methods described herein comprise an effector protein or a nucleic acid encoding the effector protein, wherein the effector protein comprises a portion of any one of the sequences recited in TABLE 1. In some embodiments, the effector protein comprises a portion of any one of the sequences recited in TABLE 1, wherein the portion does not comprise at least the first 10 amino acids, 20 amino acids, 40 amino acids, 60 amino acids, 80 amino acids, 100 amino acids, 120 amino acids, 140 amino acids, 160 amino acids, 180 amino acids, or 200 amino acids of the sequences recited in TABLE 1. In some embodiments, the effector protein comprises a portion of the sequence recited in TABLE 1, wherein the portion does not comprise the last 10 amino acids, 20 amino acids, 40 amino acids, 60 amino acids, 80 amino acids, 100 amino acids, 120 amino acids, 140 amino acids, 160 amino acids, 180 amino acids, or 200 amino acids of the sequence recited in TABLE 1. In some embodiments, the effector protein comprises a portion of any one of the sequences recited in TABLE 1, wherein the portion does not comprise at least the first 10, 20, 40, 60, 80, 100, 120, 140, 160, 180, or 200 contiguous amino acids of the sequences recited in TABLE 1. In some embodiments, the effector protein comprises a portion of the sequence recited in TABLE 1, wherein the portion does not comprise the last 10, 20, 40, 60, 80, 100, 120, 140, 160, 180, or 200 contiguous amino acids of the sequence recited in TABLE 1.

In some embodiments, compositions, systems and methods described herein comprise an effector protein or a nucleic acid encoding the effector protein, wherein the effector protein comprises an amino acid sequence that is at least 65%, at least 70%, at least 75%, at least 80%, at least 85%, at least 90%, at least 95%, at least 97%, at least 98%, at least 99%, or 100% identical to the sequence recited in TABLE 1. In some embodiments, an effector protein provided herein comprises an amino acid sequence that is at least 65% identical to the sequence recited in TABLE 1. In some embodiments, an effector protein provided herein comprises an amino acid sequence that is at least 70% identical to the sequence recited in TABLE 1. In some embodiments, an effector protein provided herein comprises an amino acid sequence that is at least 75% identical to the sequence recited in TABLE 1. In some embodiments, an effector protein provided herein comprises an amino acid sequence that is at least 80% identical to the sequence recited in TABLE 1. In some embodiments, an effector protein provided herein comprises an amino acid sequence that is at least 85% identical to the sequence recited in TABLE 1. In some embodiments, an effector protein provided herein comprises an amino acid sequence that is at least 90% identical to the sequence recited in TABLE 1. In some embodiments, an effector protein provided herein comprises an amino acid sequence that is at least 95% identical to the sequence recited in TABLE 1. In some embodiments, an effector protein provided herein comprises an amino acid sequence that is at least 98% identical to the sequence recited in TABLE 1. In some embodiments, an effector protein provided herein comprises an amino acid sequence that is at least 99% identical to the sequence recited in TABLE 1. In some embodiments, an effector protein provided herein comprises an amino acid sequence that is identical to the sequence recited in TABLE 1.

In some embodiments, the effector protein shares significant identity with SEQ ID NO: 7 but includes an amino acid substitution at a catalytic residue. For example, the catalytic residue that is not substituted is a residue of D369, E567, or D658 of SEQ ID NO: 7, when the amino acid sequence of the effector protein is aligned with SEQ ID NO: 7 for maximum sequence identity. Accordingly, in some embodiments, the effector protein does not comprise an aspartate at a residue respective of positions 369 and/or 658 of SEQ ID NO: 7 when the amino acid sequence of the effector protein is aligned with SEQ ID NO: 7 for maximum sequence identity. Alternatively, in some embodiments, the effector protein comprises an aliphatic residue (e.g., alanine, valine, glycine, leucine, isoleucine, proline) at respective positions 369 and/or 658 when the amino acid sequence of the effector protein is aligned with SEQ ID NO: 7 for maximum sequence identity. In some embodiments, the effector protein comprises glutamine or asparagine at respective positions 369 and/or 658 when the amino acid sequence of the effector protein is aligned with SEQ ID NO: 7 for maximum sequence identity. In some embodiments, the effector protein does not comprise a glutamine at a residue respective of position 567 of SEQ ID NO: 7 when the amino acid sequence of the effector protein is aligned with SEQ ID NO: 7 for maximum sequence identity. In some embodiments, the effector protein comprises an aliphatic residue (e.g., alanine, valine, glycine, leucine, isoleucine, proline) at respective position 567 when the amino acid sequence of the effector protein is aligned with SEQ ID NO: 7 for maximum sequence identity. In some embodiments, the effector protein comprises glutamine or asparagine at respective position 567 when the amino acid sequence of the effector protein is aligned with SEQ ID NO: 7 for maximum sequence identity.

In some embodiments, the effector protein comprises an amino acid substitution of D369A, wherein the effector protein has at least 75% sequence identity to the amino acid sequence of SEQ ID NO: 7. In some embodiments, the effector protein is at least 75%, at least 80%, at least 85%, at least 90%, at least 95%, at least 98%, or at least 99% identical to the amino acid sequence of SEQ ID NO: 7 and comprises an amino acid substitution of D369A. In some embodiments, the effector protein comprises an amino acid substitution of D369N. In some embodiments, the effector protein is at least 75%, at least 80%, at least 85%, at least 90%, at least 95%, at least 98%, or at least 99% identical to the amino acid sequence of SEQ ID NO: 217. In some embodiments, the effector protein is at least 75%, at least 80%, at least 85%, at least 90%, at least 95%, at least 98%, or at least 99% identical to the amino acid sequence of SEQ ID NO: 217 and comprises an amino acid substitution of D369N. In some embodiments, the effector protein comprises an amino acid substitution of E567A. In some embodiments, the effector protein is at least 75%, at least 80%, at least 85%, at least 90%, at least 95%, at least 98%, or at least 99% identical to the amino acid sequence of SEQ ID NO: 218. In some embodiments, the effector protein is at least 75%, at least 80%, at least 85%, at least 90%, at least 95%, at least 98%, or at least 99% identical to the amino acid sequence of SEQ ID NO: 218 and comprises an amino acid substitution of E567A. In some embodiments, the effector protein comprises an amino acid substitution of E567Q. In some embodiments, the effector protein is at least 75%, at least 80%, at least 85%, at least 90%, at least 95%, at least 98%, or at least 99% identical to the amino acid sequence of SEQ ID NO: 219. In some embodiments, the effector protein is at least 75%, at least 80%, at least 85%, at least 90%, at least 95%, at least 98%, or at least 99% identical to the amino acid sequence of SEQ ID NO: 219 and comprises an amino acid substitution of E567Q. In some embodiments, the effector protein comprises an amino acid substitution of D658A. In some embodiments, the effector protein is at least 75%, at least 80%, at least 85%, at least 90%, at least 95%, at least 98%, or at least 99% identical to the amino acid sequence of SEQ ID NO: 220. In some embodiments, the effector protein is at least 75%, at least 80%, at least 85%, at least 90%, at least 95%, at least 98%, or at least 99% identical to the amino acid sequence of SEQ ID NO: 220 and comprises an amino acid substitution of D658A. In some embodiments, the effector protein comprises an amino acid substitution of D658N. In some embodiments, the effector protein is at least 75%, at least 80%, at least 85%, at least 90%, at least 95%, at least 98%, or at least 99% identical to the amino acid sequence of SEQ ID NO: 221. In some embodiments, the effector protein is at least 75%, at least 80%, at least 85%, at least 90%, at least 95%, at least 98%, or at least 99% identical to the amino acid sequence of SEQ ID NO: 221 and comprises an amino acid substitution of D658N. In some embodiments, the effector protein comprises an amino acid sequence that is at least 75%, at least 80%, at least 85%, at least 90%, at least 95%, at least 99% or 100% identical to SEQ ID NO: 6 and comprises at least one amino acid substitution selected from D369A, D369N, E567A, E567Q, D658A, and D658N. In some embodiments, the effector protein comprises an amino acid sequence that is at least 75%, at least 80%, at least 85%, at least 90%, at least 95%, at least 99% or 100% identical to SEQ ID NO: 6 and comprises at least two amino acid substitution selected from D369A, D369N, E567A, E567Q, D658A, and D658N. In some embodiments, the effector protein comprises an amino acid sequence that is at least 75%, at least 80%, at least 85%, at least 90%, at least 95%, at least 99% or 100% identical to SEQ ID NO: 6 and comprises at least three amino acid substitution selected from D369A, D369N, E567A, E567Q, D658A, and D658N.

In some embodiments, the effector protein comprises an amino acid sequence that is at least 75%, at least 80%, at least 85%, at least 90%, at least 95%, at least 99% or 100% identical to SEQ ID NO: 6 and comprises at least one amino acid substitution selected from D369A, D369N, E567A, E567Q, D658A, and D658N, wherein any remaining amino acids that are different from the amino acids at respective residues of SEQ ID NO: 6 are conservative amino acids substitutions relative to SEQ ID NO: 6.

In some embodiments, the effector protein shares significant identity with SEQ ID NO: 178, but includes an amino acid substitution at a catalytic residue. In some embodiments, the catalytic residue is a respective residue of D267 of SEQ ID NO: 178, when the amino acid sequence of the effector protein is aligned with SEQ ID NO: 178 for maximum sequence identity. In some embodiments, the effector protein does not comprise an aspartate at a residue respective of position 267 of SEQ ID NO: 178 when the amino acid sequence of the effector protein is aligned with SEQ ID NO: 178 for maximum sequence identity. In some embodiments, the effector protein comprises an aliphatic residue (e.g., alanine, valine, glycine, leucine, isoleucine, proline) at respective position 267 when the amino acid sequence of the effector protein is aligned with SEQ ID NO: 178 for maximum sequence identity. In some embodiments, the effector protein comprises glutamine or asparagine at respective position 267 when the amino acid sequence of the effector protein is aligned with SEQ ID NO: 178 for maximum sequence identity.

In some embodiments, the effector protein comprises an amino acid substitution of D267A, wherein the effector protein is at least 75%, at least 80%, at least 85%, at least 90%, at least 95%, at least 98%, or at least 99% identical to the amino acid sequence of SEQ ID NO: 222. In some embodiments, the effector protein is at least 75%, at least 80%, at least 85%, at least 90%, at least 95%, at least 98%, or at least 99% identical to the amino acid sequence of SEQ ID NO: 222 and comprises an amino acid substitution of D267A. In some embodiments, the effector protein comprises an amino acid sequence that is at least 75%, at least 80%, at least 85%, at least 90%, at least 95%, at least 99% or 100% identical to SEQ ID NO: 178 and comprises at least one amino acid substitution wherein one amino acid substitution is of D267A.

In some embodiments, the effector protein comprises an amino acid sequence that is at least 75%, at least 80%, at least 85%, at least 90%, at least 95%, at least 99% or 100% identical to SEQ ID NO: 6 and comprises at least one amino acid substitution selected from D267A, wherein any remaining amino acids that are different from the amino acids at respective residues of SEQ ID NO: 178 are conservative amino acids substitutions relative to SEQ ID NO: 178.

In some embodiments, the effector protein shares significant identity with SEQ ID NO: 176, but includes an amino acid substitution at a catalytic residue. In some embodiments, the catalytic residue is a respective residue of D267 or E363 of SEQ ID NO: 176, when the amino acid sequence of the effector protein is aligned with SEQ ID NO: 176 for maximum sequence identity. In some embodiments, the effector protein does not comprise an aspartate at a residue respective of position 267 of SEQ ID NO: 176 when the amino acid sequence of the effector protein is aligned with SEQ ID NO: 176 for maximum sequence identity. In some embodiments, the effector protein does not comprise a glutamate at a residue respective of position 363 of SEQ ID NO: 176 when the amino acid sequence of the effector protein is aligned with SEQ ID NO: 176 for maximum sequence identity. In some embodiments, the effector protein comprises an aliphatic residue (e.g., alanine, valine, glycine, leucine, isoleucine, proline) at respective position 267 and/or 363 when the amino acid sequence of the effector protein is aligned with SEQ ID NO: 176 for maximum sequence identity. In some embodiments, the effector protein comprises glutamine or asparagine at respective position 267 and/or 363 when the amino acid sequence of the effector protein is aligned with SEQ ID NO: 176 for maximum sequence identity.

In some embodiments, the effector protein comprises an amino acid substitution of D267A, wherein the effector protein is at least 75%, at least 80%, at least 85%, at least 90%, at least 95%, at least 98%, or at least 99% identical to the amino acid sequence of SEQ ID NO: 223. In some embodiments, the effector protein is at least 75%, at least 80%, at least 85%, at least 90%, at least 95%, at least 98%, or at least 99% identical to the amino acid sequence of SEQ ID NO: 223 and comprises an amino acid substitution of D267A. In some embodiments, the effector protein comprises an amino acid substitution of D267N. In some embodiments, the effector protein is at least 75%, at least 80%, at least 85%, at least 90%, at least 95%, at least 98%, or at least 99% identical to the amino acid sequence of SEQ ID NO: 224. In some embodiments, the effector protein is at least 75%, at least 80%, at least 85%, at least 90%, at least 95%, at least 98%, or at least 99% identical to the amino acid sequence of SEQ ID NO: 224 and comprises an amino acid substitution of D267N. In some embodiments, the effector protein comprises an amino acid substitution of E363Q. In some embodiments, the effector protein is at least 75%, at least 80%, at least 85%, at least 90%, at least 95%, at least 98%, or at least 99% identical to the amino acid sequence of SEQ ID NO: 225. In some embodiments, the effector protein is at least 75%, at least 80%, at least 85%, at least 90%, at least 95%, at least 98%, or at least 99% identical to the amino acid sequence of SEQ ID NO: 225 and comprises an amino acid substitution of E363Q. In some embodiments, the effector protein comprises an amino acid sequence that is at least 75%, at least 80%, at least 85%, at least 90%, at least 95%, at least 99% or 100% identical to SEQ ID NO: 176 and comprises at least one amino acid substitution selected from D267A, D267N, and E363Q. In some embodiments, the effector protein comprises an amino acid sequence that is at least 75%, at least 80%, at least 85%, at least 90%, at least 95%, at least 99% or 100% identical to SEQ ID NO: 176 and comprises at least two amino acid substitution selected from D267A, D267N, and E363Q.

In some embodiments, the effector protein comprises an amino acid sequence that is at least 75%, at least 80%, at least 85%, at least 90%, at least 95%, at least 99% or 100% identical to SEQ ID NO: 176 and comprises at least one amino acid substitution selected from D267A, D267N, and E363Q, wherein any remaining amino acids that are different from the amino acids at respective residues of SEQ ID NO: 176 are conservative amino acids substitutions relative to SEQ ID NO: 176.

In some embodiments, the effector protein comprises an amino acid substitution of D326A, wherein the effector protein is at least 75%, at least 80%, at least 85%, at least 90%, at least 95%, at least 98%, or at least 99% identical to the amino acid sequence of SEQ ID NO: 226. In some embodiments, the effector protein is at least 75%, at least 80%, at least 85%, at least 90%, at least 95%, at least 98%, or at least 99% identical to the amino acid sequence of SEQ ID NO: 226 and comprises an amino acid substitution of D326A.

In some embodiments, the effector protein is at least 75%, at least 80%, at least 85%, at least 90%, at least 95%, at least 98%, or at least 99% identical to the amino acid sequence of SEQ ID NO: 226, and comprises an aliphatic residue at position 326. In some embodiments, the effector protein comprises an amino acid sequence that is at least 75%, at least 80%, at least 85%, at least 90%, at least 95%, at least 99% or 100% identical to SEQ ID NO: 226 and comprises at least one amino acid substitution selected from D267A, D267N, and E363Q, wherein any remaining amino acids that are different from the amino acids at respective residues of SEQ ID NO: 226 are conservative amino acids substitutions relative to SEQ ID NO: 226.

In some embodiments, the effector proteins cause indel formation in the target nucleic acids. Such an indel can result in a deletion of one or more nucleotides. In some embodiments, the indel is a type of genetic mutation that results from the insertion and/or deletion of nucleotides in a target nucleic acid. An indel can vary in length (e.g., 1 to 1,000 nucleotides in length) and be detected using methods well known in the art, including sequencing. If the number of nucleotides in the insertion/deletion is not divisible by three, and it occurs in a protein coding region, it may result in a frameshift mutation. In some embodiments an indel refers to a length difference between two alleles. In some embodiments, indel occurrence in target nucleic acids is mitigated by use of a catalytically inactive effector protein.

In some embodiments, the effector protein may cleave nucleic acids, including single stranded RNA (ssRNA), double stranded DNA (dsDNA), and single-stranded DNA (ssDNA). In some embodiments, the effector protein nicks the target nucleic acid. In some embodiments, the target nucleic acid is a single stranded RNA (ssRNA), double stranded DNA (dsDNA), or single-stranded DNA (ssDNA). In some embodiments, the target nucleic acid is a double stranded DNA. In some embodiments, the double stranded DNA comprises a target strand and a non-target strand. In some embodiments, the effector protein nicks the target strand of the double stranded DNA molecule. In some embodiments, the effector protein has been engineered to nick the target strand of the double stranded DNA molecule. In some embodiments, the effector protein performs a double stranded break (DSB). In some embodiments, the DSB is created by two single stranded breaks.

### Fusion Partners

Provided herein are fusion effector proteins that comprise at least one fusion partner. In some embodiments, fusion partners provide enzymatic activity that modifies a target nucleic acid. In some embodiments, the fusion partner protein is fused to the 5′ end of the effector protein. In some embodiments, the fusion partner protein is fused to the 3′ end of the effector protein. In some embodiments, the effector protein is located at an internal location of the fusion partner protein. In some embodiments, the fusion partner protein is located at an internal location of the Cas effector protein. For example, a base editing enzyme (e.g., a deaminase enzyme) is inserted at an internal location of a Cas effector protein. The effector protein may be fused directly or indirectly (e.g., via a linker) to the fusion partner protein. Exemplary linkers are described herein.

In some embodiments, a fusion protein described herein comprises 1, 2, 3, 4, 5, 6, 7, 8, 9, 10, or more fusion partners at or near the N-terminus, 1, 2, 3, 4, 5, 6, 7, 8, 9, 10, or more fusion partners at or near the C-terminus, or a combination of these (e.g., one or more fusion partners at the amino-terminus and one or more fusion partners at the carboxy terminus). When more than one fusion partner is present, each may be selected independently of the others, such that a single fusion partner may be present in more than one copy and/or in combination with one or more other fusion partners present in one or more copies.

In some embodiments, fusion partners provide enzymatic activity that modifies expression of a target nucleic acid. The target nucleic acid may be a gene. The target nucleic acid may be DNA. The target nucleic acid may be RNA. Such enzymatic activities include, but are not limited to, nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, and glycosylase activity. Examples of enzymatic activity that modifies the target nucleic acid include, but are not limited to: nuclease activity such as that provided by a restriction enzyme (e.g., FokI nuclease); methyltransferase activity such as that provided by a methyltransferase (e.g., HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants)); demethylase activity such as that provided by a demethylase (e.g., Ten-Eleven Translocation (TET) dioxygenase 1 (TET1CD), TET1, DME, DML1, DML2, ROS1); DNA repair activity; DNA damage (e.g., oxygenation) activity; deamination activity such as that provided by a deaminase (e.g., a cytosine deaminase enzyme such as rat APOBEC1); dismutase activity; alkylation activity; depurination activity; oxidation activity; pyrimidine dimer forming activity; integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase); transposase activity, recombinase activity such as that provided by a recombinase (e.g., catalytic domain of Gin recombinase); as well as polymerase activity, ligase activity, helicase activity, photolyase activity, and glycosylase activity.

In some embodiments, fusion partners have enzymatic activity that modifies a protein associated with a target nucleic acid. The protein may be a histone, an RNA binding protein, or a DNA binding protein. Such enzymatic activities include, but are not limited to, methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, de-ribosylation activity, myristoylation activity, and demyristoylation activity. Examples of such enzymatic activities include methyltransferase activity such as that provided by a histone methyltransferase (HMT) (e.g., suppressor of variegation 3-9 homolog 1 (SUV39H1, also known as KMT1A), euchromatic histone lysine methyltransferase 2 (G9A, also known as KMT1C and EHMT2), SUV39H2, ESET/SETDB1, SET1A, SET1B, MLL1 to 5, ASH1, SYMD2, NSD1, DOT1L, Pr-SET7/8, SUV4-20H1, EZH2, RIZ1); demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3); acetyltransferase activity such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HBO1/MYST2, HMOF/MYST1, SRC1, ACTR, P160, CLOCK); deacetylase activity such as that provided by a histone deacetylase (e.g., HDAC1, HDAC2, HDAC3, HDAC8, HDAC4, HDAC5, HDAC7, HDAC9, SIRT1, SIRT2, HDAC11); kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity.

In some embodiments, fusion partners may comprise a protein or domain thereof selected from: endonucleases (e.g., RNase III, the CRR22 DYW domain, Dicer, and PIN (PilT N-terminus); SMG5 and SMG6; domains responsible for stimulating RNA cleavage (e.g., CPSF, CstF, CFIm and CFIIm); exonucleases such as XRN-1 or Exonuclease T; deadenylases such as HNT3; protein domains responsible for nonsense mediated RNA decay (e.g., UPF1, UPF2, UPF3, UPF3b, RNP S1, Y14, DEK, REF2, and SRm160); protein domains responsible for stabilizing RNA (e.g., PABP); proteins and protein domains responsible for polyadenylation of RNA (e.g., PAP1, GLD-2, and Star-PAP); proteins and protein domains responsible for polyuridinylation of RNA (e.g., CI D1 and terminal uridylate transferase); and other suitable domains that affect nucleic acid modification.

In some embodiments, fusion partners may comprise a chromatin-modifying enzyme. In some embodiments, the fusion partner may chemically modify a target nucleic acid, for example by methylating, demethylating, or acetylating the target nucleic acid in a sequence specific or non-specific manner.

It is understood that a fusion partner may comprise an entire protein or a functional fragment of the protein (e.g., a functional domain). In some embodiments, the functional domain interacts with or binds a target nucleic acid, including intramolecular and/or intermolecular secondary structures thereof, e.g., hairpins, stem-loops, etc. The functional domain may interact transiently or irreversibly, directly or indirectly with a target nucleic acid. In some embodiments, the functional domain has nuclease activity. A functional domain may be a domain of a protein selected from the group comprising endonucleases; proteins and protein domains capable of stimulating RNA cleavage; exonucleases; deadenylases; proteins and protein domains having nonsense mediated RNA decay activity; proteins and protein domains capable of stabilizing RNA; proteins and protein domains capable of repressing translation; proteins and protein domains capable of stimulating translation; proteins and protein domains capable of modulating translation (e.g., translation factors such as initiation factors, elongation factors, release factors, etc., e.g., eIF4G); proteins and protein domains capable of polyadenylation of RNA; proteins and protein domains capable of polyuridinylation of RNA; proteins and protein domains having RNA localization activity; proteins and protein domains capable of nuclear retention of RNA; proteins and protein domains having RNA nuclear export activity; proteins and protein domains capable of repression of RNA splicing; proteins and protein domains capable of stimulation of RNA splicing; proteins and protein domains capable of reducing the efficiency of transcription; and proteins and protein domains capable of stimulating transcription.

A fusion protein described herein may comprise a GLP-1 polypeptide, a GLP-1 fragment, or GLP-1 variant, which can be functionally important for stimulating insulin secretion. The GLP-1 polypeptide, fragment, or variant thereof may be fused to an albumin polypeptide, fragment, or variant thereof as described in U.S. Pat. No. 7,141,547, which is incorporated by reference in its entirety.

In some embodiments, a fusion partner may provide signaling activity. In some embodiments, a fusion partner may inhibit or promote the formation of multimeric complex of an effector protein. In an additional example, the fusion partner may directly or indirectly modify a target nucleic acid. Modifications can be of a nucleobase, nucleotide, or nucleotide sequence of a target nucleic acid. In some embodiments, the fusion partner may interact with additional proteins, or functional fragments thereof, to make modifications to a target nucleic acid. In other embodiments, the fusion partner may modify proteins associated with a target nucleic acid. In some embodiments, a fusion partner may modulate transcription (e.g., inhibits transcription, increases transcription) of a target nucleic acid. In yet another example, a fusion partner may directly or indirectly inhibit, reduce, activate or increase expression of a target nucleic acid. By way of non-limiting example, the fusion protein may comprise an effector protein described herein and a fusion partner comprising a Calcineurin A tag, wherein the fusion protein dimerizes in the presence of Tacrolimus (FK506). Also, by way of non-limiting example, the fusion protein may comprise an effector protein described herein and a SpyTag configured to dimerize or associate with another effector protein in a multimeric complex. Multimeric complex formation is further described herein.

In some embodiments, fusion partners comprise an amino acid sequence that is at least 50%, at least 55%, at least 60%, at least 65%, at least 70%, at least 75%, at least 80%, at least 85%, at least 90%, at least 95%, at least 98%, at least 99% or 100% identical to a fusion partner disclosed in TABLE 2. In some embodiments, compositions and methods comprise a fusion partner, wherein the amino acid sequence of the fusion partner is at least 50%, at least 55%, at least 60%, at least 65%, at least 70%, at least 75%, at least 80%, at least 85%, at least 90%, at least 95%, at least 98%, at least 99% or 100% identical to a fusion partner disclosed in TABLE 2.

TABLE 2
Exemplary Fusion Partner Sequences

| Nickname | SEQ | |
|---|---|---|
| (typical | ID | |
| function) | NO: | Sequence |
| ABE8e | 400 | SEVEFSHEYWMRHALTLAKRARDEREVPVGAVLVLNNRVIGEG |
| (base editor) | | WNRAIGLHDPTAHAEIMALRQGGLVMQNYRLIDATLYVTFEPC |
| | | VMCAGAMIHSRIGRVVFGVRNSKRGAAGSLMNVLNYPGMNHR |
| | | VEITEGILADECAALLCDFYRMPRQVFNAQKKAQSSIN |
| ABE8e-TadA | 421 | SEVEFSHEYWMRHALTLAKRARDEREVPVGAVLVLNNRVIGEG |
| (base editor) | | WNRAIGLHDPTAHAEIMALRQGGLVMQNYRLIDATLYVTFEPC |
| | | VMCAGAMIHSRIGRVVFGVRNSKRGAAGSLMNVLNYPGMNHR |
| | | VEITEGILADECAALLCDFYRMPRQVFNAQKKAQSSINSGGSSG |
| | | GSSGSETPGTSESATPESSGGSSGGSSEVEFSHEYWMRHALTLAK |
| | | RAWDEREVPVGAVLVHNNRVIGEGWNRPIGRHDPTAHAEIMAL |
| | | RQGGLVMQNYRLIDATLYVTLEPCVMCAGAMIHSRIGRVVFGA |
| | | RDAKTGAAGSLMDVLHHPGMNHRVEITEGILADECAALLSDFF |
| | | RMRRQEIKAQKKAQSSTD |
| TadA-ABE8e | 422 | SEVEFSHEYWMRHALTLAKRAWDEREVPVGAVLVHNNRVIGE |
| (base editor) | | GWNRPIGRHDPTAHAEIMALRQGGLVMQNYRLIDATLYVTLEP |
| | | CVMCAGAMIHSRIGRVVFGARDAKTGAAGSLMDVLHHPGMNH |
| | | RVEITEGILADECAALLSDFFRMRRQEIKAQKKAQSSTDSGGSSG |
| | | GSSGSETPGTSESATPESSGGSSGGSSEVEFSHEYWMRHALTLAK |
| | | RARDEREVPVGAVLVLNNRVIGEGWNRAIGLHDPTAHAEIMAL |
| | | RQGGLVMQNYRLIDATLYVTFEPCVMCAGAMIHSRIGRVVFGV |
| | | RNSKRGAAGSLMNVLNYPGMNHRVEITEGILADECAALLCDFY |
| | | RMPRQVFNAQKKAQSSIN |
| ABE8.20m | 401 | SEVEFSHEYWMRHALTLAKRARDEREVPVGAVLVLNNRVIGEG |
| (base editor) | | WNRAIGLHDPTAHAEIMALRQGGLVMQNYRLYDATLYSTFEPC |
| | | VMCAGAMIHSRIGRVVFGVRNAKTGAAGSLMDVLHHPGMNHR |
| | | VEITEGILADECAALLCRFFRMPRRVFNAQKKAQSSTD |
| APOBEC3A | 402 | EASPASGPRHLMDPHIFTSNFNNGIGRHKTYLCYEVERLDNGTS |
| (base editor or | | VKMDQHRGFLHNQAKNLLCGFYGRHAELRFLDLVPSLQLDPAQ |
| deaminase) | | IYRVTWFISWSPCFSWGCAGEVRAFLQENTHVRLRIFAARIYDY |
| | | DPLYKEALQMLRDAGAQVSIMTYDEFKHCWDTFVDHQGCPFQP |
| | | WDGLDEHSQALSGRLRAILQNQGN |
| Anc APOBEC | 403 | SSETGPVAVDPTLRRRIEPHEFEVFFDPRELRKETCLLYEIKWGTS |
| (a.k.a. | | HKIWRHSSKNTTKHVEVNFIEKFTSERHFCPSTSCSITWFLSWSPC |
| AncBE4Max) | | GECSKAITEFLSQHPNVTLVIYVARLYHHMDQQNRQGLRDLVNS |
| (base editor or | | GVTIQIMTAPEYDYCWRNFVNYPPGKEAHWPRYPPLWMKLYA |
| deaminase) | | LELHAGILGLPPCLNILRRKQPQLTFFTIALQSCHYQRLPPHILWA |
| | | TGLK |
| BtAPOBEC2 | 404 | MAQKEEAAAAAEPASQNGEEVENLEDPEKLKELIELPPFEIVTGE |
| (base editor or | | RLPAHYFKFQFRNVEYSSGRNKTFLCYVVEAQSKGGQVQASRG |
| deaminase) | | YLEDEHATNHAEEAFFNSIMPTFDPALRYMVTWYVSSSPCAACA |
| | | DRIVKTLNKTKNLRLLILVGRLFMWEEPEIQAALRKLKEAGCRL |
| | | RIMKPQDFEYIWQNFVEQEEGESKAFEPWEDIQENFLYYEEKLA |
| | | DILK |
| M-MLV RT | 405 | TLNIEDEYRLHETSKEPDVSLGSTWLSDFPQAWAETGGMGLAVR |
| or mutant | | QAPLIIPLKATSTPVSIKQYPMSQEARLGIKPHIQRLLDQGILVPCQ |
| thereof | | SPWNTPLLPVKKPGTNDYRPVQDLREVNKRVEDIHPTVPNPYNL |
| (D200N/L603W/ | | LSGLPPSHQWYTVLDLKDAFFCLRLHPTSQPLFAFEWRDPEMGI |
| T330P/T306K/ | | SGQLTWTRLPQGFKNSPTLFNEALHRDLADFRIQHPDLILLQYVD |
| W313F | | DLLLAATSELDCQQGTRALLQTLGNLGYRASAKKAQICQKQVK |
| mutations) | | YLGYLLKEGQRWLTEARKETVMGQPTPKTPRQLREFLGKAGFC |
| (prime | | RLFIPGFAEMAAPLYPLTKPGTLFNWGPDQQKAYQEIKQALLTA |
| editing) | | PALGLPDLTKPFELFVDEKQGYAKGVLTQKLGPWRRPVAYLSK |
| | | KLDPVAAGWPPCLRMVAAIAVLTKDAGKLTMGQPLVILAPHAV |
| | | EALVKQPPDRWLSNARMTHYQALLLDTDRVQFGPVVALNPATL |
| | | LPLPEEGLQHNCLDILAEAHGTRPDLTDQPLPDADHTWYTDGSS |
| | | LLQEGQRKAGAAVTTETEVIWAKALPAGTSAQRAELIALTQALK |
| | | MAEGKKLNVYTDSRYAFATAHIHGEIYRRRGWLTSEGKEIKNK |
| | | DEILALLKALFLPKRLSIIHCPGHQKGHSAEARGNRMADQAARK |
| | | AAITETPDTSTLLIENSSP |
| TET1 | 406 | MASNFTQFVLVDNGGTGDVTVAPSNFANGVAEWISSNSRSQAY |
| (CRISPRa) | | KVTCSVRQSSAQKRKYTIKVEVPKVATQTVGGVELPVAAWRSY |
| | | LNMELTIPIFATNSDCELIVKAMQGLLKDGNPIPSAIAANSGIYSA |
| | | GGGGSGGGGSGGGGSPKKKRKVEASGGGSGGGSGGGSGEAAP |
| | | CDCDGGTQKEKGPYYTHLGAGPSVAAVRELMETRFGQKGKAIR |
| | | IEKIVFTGKEGKSSQGCPVAKWVIRRSGPEEKLICLVRERVDHHC |
| | | STAVIVVLILLWEGIPRLMADRLYKELTENLRSYSGHPTDRRCTL |
| | | NKKRTCTCQGIDPKTCGASFSFGCSWSMYFNGCKFGRSENPRKF |
| | | RLAPNYPLHNYYKRITGMSSEGSDVKTGWIIPDRKTLISREEKQL |
| | | EKNLQELATVLAPLYKQMAPVAYQNQVEYEEVAGDCRLGNEE |
| | | GRPFSGVTCCMDFCAHSHKDIHNMHNGSTVVCTLIRADGRDTN |
| | | CPEDEQLHVLPLYRLADTDEFGSVEGMKAKIKSGAIQVNGPTRK |
| | | RRLRFTEPVPRCGKRAKMKQNHNKSGSHNTKSFSSASSTSHLVK |
| | | DESTDFCPLQASSAETSTCTYSKTASGGFAETSSILHCTMPSGAH |
| | | SGANAAAGECTGTVQPAEVAAHPHQSLPTADSPVHAEPLTSPSE |
| | | QLTSNQSNQQLPLLSNSQKLASCQVEDERHPEADEPQHPEDDNL |
| | | PQLDEFWSDSEEIYADPSFGGVAIAPIHGSVLIECARKELHATTSL |
| | | RSPKRGVPFRVSLVFYQHKSLNKPNHGFDINKIKCKCKKVTKKK |
| | | PADRECPDVSPEANLSHQIPSRVASTLTRDNVVTVSPYSLTHVAG |
| | | PYNRWVAAADYKDDDDK |
| TET2 | 407 | QSQPGHNQMLRPIKTEPVSKPSSYRYPLSPPQENMSSRIKQEISSP |
| (CRISPRa) | | SRDNGQPKSIIETMEQHLKQFQLKSLCDYKALTLKSQKHVKVPT |
| | | DIQAAESENHARAAEPQATKSTDCSVLDDVSESDTPGEQSQNGK |
| | | CEGCNPDKDEAPYYTHLGAGPDVAAIRTLMEERYGEKGKAIRIE |
| | | KVIYTGKEGKSSQGCPIAKWVYRRSSEEEKLLCLVRVRPNHTCE |
| | | TAVMVIAIMLWDGIPKLLASELYSELTDILGKCGICTNRRCSQNE |
| | | TKKKQSPPRNCCCQGENPETCGASFSFGCSWSMYYNGCKFARS |
| | | KKPRKFRLHGAEPKEEERLGSHLQNLATVIAPIYKKLAPDAYNN |
| | | QVEFEHQAPDCCLGLKEGRPFSGVTACLDFSAHSHRDQQNMPN |
| | | GSTVVVTLNREDNREVGAKPEDEQFHVLPMYIIAPEDEFGSTEG |
| | | QEKKIRMGSIEVLQSFRRRRVIRIGELPKSCKKKAEPKKAKTKKA |
| | | ARKHSSLENCSSRTEKGKSSSHTKLMENASHMKQMTAQPQLSG |
| | | PVIRQPPTLQRHLQQGQRPQQPQPPQPQPQTTPQPQPQPQHIMPG |
| | | NSQSVGSHCSGSTSVYTRQPTPHSPYPSSAHTSDIYGDTNHVNFY |
| | | PTSSHASGSYLNPSNYMNPYLGLLNQNNQYAPFPYNGSVPVDN |
| | | GSPFLGSYSPQAQSRDLHRYPNQDHLTNQNLPPIHTLHQQTFGDS |
| | | PSKYLSYGNQNMQRDAFTTNSTLKPNVHHLATFSPYPTPKMDS |
| | | HFMGAASRSPYSHPHTDYKTSEHHLPSHTVYSYTAAASGSSSSH |
| | | AFHNKENDNIANGLSRVLPGFNHDRTASAQELLYSLTGSSQEKQ |
| | | PEVSGQDAAAVQEIEYWSDSEHNFQDPCIGGVAIAPTHGSILIEC |
| | | AKCEVHATTKVNDPDRNHPTRISLVLYRHKNLFLPKHCLALWE |
| | | AKMAEKARKEEECGKNGSDHVSQKNHGKQEKREPTGPQEPSYL |
| | | RFIQSLAENTGSVTTDSTVTTSPYAFTQVTGPYNTFV |
| P300 | 408 | IFKPEELRQALMPTLEALYRQDPESLPFRQPVDPQLLGIPDYFDIV |
| (CRISPRa) | | KSPMDLSTIKRKLDTGQYQEPWQYVDDIWLMFNNAWLYNRKT |
| | | SRVYKYCSKLSEVFEQEIDPVMQSLGYCCGRKLEFSPQTLCCYG |
| | | KQLCTIPRDATYYSYQNRYHFCEKCFNEIQGESVSLGDDPSQPQT |
| | | TINKEQFSKRKNDTLDPELFVECTECGRKMHQICVLHHEIIWPAG |
| | | FVCDGCLKKSARTRKENKFSAKRLPSTRLGTFLENRVNDFLRRQ |
| | | NHPESGEVTVRVVHASDKTVEVKPGMKARFVDSGEMAESFPYR |
| | | TKALFAFEEIDGVDLCFFGMHVQEYGSDCPPPNQRRVYISYLDS |
| | | VHFFRPKCLRTAVYHEILIGYLEYVKKLGYTTGHIWACPPSEGD |
| | | DYIFHCHPPDQKIPKPKRLQEWYKKMLDKAVSERIVHDYKDIFK |
| | | QATEDRLTSAKELPYFEGDFWPNVLEESIKELEQEEEERKREENT |
| | | SNESTDVTKGDSKNAKKKNNKKTSKNKSSLSRGNKKKPGMPNV |
| | | SNDLSQKLYATMEKHKEVFFVIRLIAGPAANSLPPIVDPDPLIPCD |
| | | LMDGRDAFLTLARDKHLEFSSLRRAQWSTMCMLVELHTQSQD |
| VPR | 409 | DALDDFDLDMLGSDALDDFDLDMLGSDALDDFDLDMLGSDAL |
| (CRISPRa) | | DDFDLDMLINSRSSGSPKKKRKVGSQYLPDTDDRHRIEEKRKRT |
| | | YETFKSIMKKSPFSGPTDPRPPPRRIAVPSRSSASVPKPAPQPYPFT |
| | | SSLSTINYDEFPTMVFPSGQISQASALAPAPPQVLPQAPAPAPAPA |
| | | MVSALAQAPAPVPVLAPGPPQAVAPPAPKPTQAGEGTLSEALLQ |
| | | LQFDDEDLGALLGNSTDPAVFTDLASVDNSEFQQLLNQGIPVAP |
| | | HTTEPMLMEYPEAITRLVTGAQRPPDPAPAPLGAPGLPNGLLSG |
| | | DEDFSSIADMDFSALLGSGSGSRDSREGMFLPKPEAGSAISDVFE |
| | | GREVCQPKRIRPFHPPGSPWANRPLPASLAPTPTGPVHEPVGSLT |
| | | PAPVPQPLDPAPAVTPEASHLLEDPDEETSQAVKALREMADTVIP |
| | | QKEEAAICGQMDLSHPPPRGHLDELTTTLESMTEDLNLDSPLTPE |
| | | LNEILDTFLNDECLLHAMHISTGLSIFDTSLF |
| DNMT3A | 410 | TYGLLRRREDWPSRLQMFFANNHDQEFDPPKVYPPVPAEKRKPI |
| (CRISPRi) | | RVLSLFDGIATGLLVLKDLGIQVDRYIASEVCEDSITVGMVRHQG |
| | | KIMYVGDVRSVTQKHIQEWGPFDLVIGGSPCNDLSIVNPARKGL |
| | | YEGTGRLFFEFYRLLHDARPKEGDDRPFFWLFENVVAMGVSDK |
| | | RDISRFLESNPVMIDAKEVSAAHRARYFWGNLPGMNRPLASTVN |
| | | DKLELQECLEHGRIAKFSKVRTITTRSNSIKQGKDQHFPVFMNEK |
| | | EDILWCTEMERVFGFPVHYTDVSNMSRLARQRLLGRSWSVPVIR |
| | | HLFAPLKEYFACV |
| DNMT3L | 411 | MAAIPALDPEAEPSMDVILVGSSELSSSVSPGTGRDLIAYEVKAN |
| (CRISPRi) | | QRNIEDICICCGSLQVHTQHPLFEGGICAPCKDKFLDALFLYDDD |
| | | GYQSYCSICCSGETLLICGNPDCTRCYCFECVDSLVGPGTSGKVH |
| | | AMSNWVCYLCLPSSRSGLLQRRRKWRSQLKAFYDRESENPLEM |
| | | FETVPVWRRQPVRVLSLFEDIKKELTSLGFLESGSDPGQLKHVV |
| | | DVTDTVRKDVEEWGPFDLVYGATPPLGHTCDRPPSWYLFQFHR |
| | | LLQYARPKPGSPRPFFWMFVDNLVLNKEDLDVASRFLEMEPVTI |
| | | PDVHGGSLQNAVRVWSNIPAIRSRHWALVSEEELSLLAQNKQSS |
| | | KLAAKWPTKLVKNCFLPLREYFKYFST |
| VP64 | 412 | DALDDFDLDMLGSDALDDFDLDMLGSDALDDFDLDMLGSDAL |
| (CRISPRa) | | DDFDLDML |
| EZH2 | 413 | GQTGKKSEKGPVCWRKRVKSEYMRLRQLKRFRRADEVKTMFS |
| (CRISPRi) | | SNRQKILERTETLNQEWKQRRIQPVHIMTSVSSLRGTRECSVTSD |
| | | LDFPAQVIPLKTLNAVASVPIMYSWSPLQQNFMVEDETVLHNIP |
| | | YMGDEVLDQDGTFIEELIKNYDGKVHGDRECGFINDEIFVELVN |
| | | ALGQYNDDDDDDDGDDPDEREEKQKDLEDNRDDKETCPPRKFP |
| | | ADKIFEAISSMFPDKGTAEELKEKYKELTEQQLPGALPPECTPNID |
| | | GPNAKSVQREQSLHSFHTLFCRRCFKYDCFLHPFHATPNTYKRK |
| | | NTETALDNKPCGPQCYQHLEGAKEFAAALTAERIKTPPKRPGGR |
| | | RRGRLPNNSSRPSTPTISVLESKDTDSDREAGTETGGENNDKEEE |
| | | EKKDETSSSSEANSRCQTPIKMKPNIEPPENVEWSGAEASMFRVL |
| | | IGTYYDNFCAIARLIGTKTCRQVYEFRVKESSIIAPVPTEDVDTPP |
| | | RKKKRKHRLWAAHCRKIQLKKDGSSNHVYNYQPCDHPRQPCD |
| | | SSCPCVIAQNFCEKFCQCSSECQNRFPGCRCKAQCNTKQCPCYL |
| | | AVRECDPDLCLTCGAADHWDSKNVSCKNCSIQRGSKKHLLLAP |
| | | SDVAGWGIFIKDPVQKNEFISEYCGEIISQDEADRRGKVYDKYM |
| | | CSFLFNLNNDFVVDATRKGNKIRFANHSVNPNCYAKVMMVNG |
| | | DHRIGIFAKRAIQTGEELFFDYRYSQADALKYVGIEREMEIP |
| KRAB/ | 414 | MDAKSLTAWSRTLVTFKDVFVDFTREEWKLLDTAQQIVYRNV |
| KOX1 | | MLENYKNLVSLGYQLTKPDVILRLEKGEEPWLV |
| (CRISPRi) | | |
| ZIM3 | 415 | NNSQGRVTFEDVTVNFTQGEWQRLNPEQRNLYRDVMLENYSNL |
| (CRISPRi) | | VSVGQGETTKPDVILRLEQGKEPWLEEEEVLGSGRAEKNGDIGG |
| | | QIWKPKDVKESL |
| Tn5 | 416 | VGTMITSALHRAADWAKSVFSSAALGDPRRTARLVNVAAQLAK |
| transposase | | YSGKSITISSEGSKAMQEGAYRFIRNPNVSAEAIRKAGAMQTVKL |
| (DNA | | AQEFPELLAIEDTTSLSYRHQVAEKLGKLGSIQDKSRGWWVHSV |
| transposition; | | LLLEATTFRTVGLLHQEWWMRPDDPADADEKESGKWLAAAAT |
| targeted gene | | SRLRMGSMMSNVIAVCDREADIHAYLQDKLAHNERFVVRSKHP |
| insertion) | | RKDVESGLYLYDHLKNQPELGGYQISIAQKGVVDKRGKRKNRP |
| | | ARKASLSLRSGRITLKQGNITLNAVLAEEINPPKGETPLKWLLLTS |
| | | EPVESLAQALRVIDIYTHRWRIEEFHKAWKTGAGAERQRMEEPD |
| | | NLERMVSILSFVAVRLLQLRESFTPPQALRAQGLLKEAEHVESQS |
| | | AETVLTPDECQLLGYLDKGKRKRKEKAGSLQWAYMAIARLGGF |
| | | MDSKRTGIASWGALWEGWEALQSKLDGFLAAKDLMAQGIKI |
| SB100X | 417 | MGKSKEISQDLRKRIVDLHKSGSSLGAISKRLAVPRSSVQTIVRK |
| (DNA | | YKHHGTTQPSYRSGRRRVLSPRDERTLVRKVQINPRTTAKDLVK |
| transposition; | | MLEETGTKVSISTVKRVLYRHNLKGHSARKKPLLQNRHKKARL |
| targeted gene | | RFATAHGDKDRTFWRNVLWSDETKIELFGHNDHRYVWRKKGE |
| insertion) | | ACKPKNTIPTVKHGGGSIMLWGCFAAGGTGALHKIDGIMDAVQ |
| | | YVDILKQHLKTSVRKLKLGRKWVFQHDNDPKHTSKVVAKWLK |
| | | DNKVKVLEWPSQSPDLNPIENLWAELKKRVRARRPTNLTQLHQ |
| | | LCQEEWAKIHPNYCGKLVEGYPKRLTQVKQFKGNATKY |
| Phage | 418 | MPIAPEFLSLAYPGQEFPAYLYGRASRDPKRKGRSVQSQLDEGR |
| encoded | | ATCLDAGWPIAGEFKDVDRSASAYARRTRDEFEEMIAGIQAGEC |
| serine | | RILVAFEASRYYRDLEAYVRLRRVCREAGVLLCYNGQVYDLSK |
| integrases/ | | SADRKATAQDAVNAEGEADDIRERNLRTTRLNAKRGGAHGPVP |
| recombinase 2 | | DGYKRRYDPDSGDLVDQIPHPDRAGLITEIFRRAAAAEPLAAICR |
| (DNA | | DLNERGETTHRGKAWQRHHLHAILRNPAYIGHRRHLGVDTGKG |
| transposition; | | MWAPICDDEDFAETFQAVQEILSLPGRQLSPGPEAQHLQTGIALC |
| targeted gene | | GEHPDEPPLRSVTVRGRTNYNCSTRYDVAMREDRMDAFVEESV |
| insertion) | | ITWLASDEAVAAFEDNTDDERTRKARIRLKVLEEQLEAAQKQAR |
| | | TLRPDGMGMLLSIDSLAGLEAELTPQIDKARQESRSLHVPALLRD |
| | | LLGKPRADVDRAWNEALTLPQRRMILRMVVTIRLFKAGSRGVR |
| | | AIEPGRITLSYVGEPGFKPVGGNRAKQ |
| Phage | 419 | MAVGIYIRVSTQEQASEGHSIESQKKKLASYCEIQGWDDYRFYIE |
| encoded | | EGISGKNTNRPKLKLLMEHIEKGKINILLVYRLDRLTRSVIDLHKL |
| serine | | LNFLQEHGCAFKSATETYDTTTANGRMSMGIVSLLAQWETENM |
| integrase/ | | SERIKLNLEHKVLVEGERVGAIPYGFDLSDDEKLVKNEKSAILLD |
| recombinase | | MVERVENGWSVNRIVNYLNLTNNDRNWSPNGVLRLLRNPALY |
| 13 (DNA | | GATRWNDKIAENTHEGIISKERFNRLQQILADRSIHHRRDVKGTY |
| transposition; | | IFQGVLRCPVCDQTLSVNRFIKKRKDGTEYCGVLYRCQPCIKQN |
| targeted gene | | KYNLAIGEARFLKALNEYMSTVEFQTVEDEVIPKKSEREMLESQ |
| insertion) | | LQQIARKREKYQKAWASDLMSDDEFEKLMVETRETYDECKQKL |
| | | ESCEDPIKIDETYLKEIVYMFHQTFNDLESEKQKEFISKFIRTIRYT |
| | | VKEQQPIRPDKSKTGKGKQKVIITEVEFYQ |
| Human WT | 420 | MGIQGLLQFIKEASEPIHVRKYKGQVVAVDTYCWLHKGAIACAE |
| Exonuclease | | KLAKGEPTDRYVGFCMKFVNMLLSHGIKPILVFDGCTLPSKKEV |
| 1a (DNA | | ERSRRERRQANLLKGKQLLREGKVSEARECFTRSINITHAMAHK |
| transposition; | | VIKAARSQGVDCLVAPYEADAQLAYLNKAGIVQAIITEDSDLLA |
| targeted gene | | FGCKKVILKMDQFGNGLEIDQARLGMCRQLGDVFTEEKFRYMC |
| insertion) | | ILSGCDYLSSLRGIGLAKACKVLRLANNPDIVKVIKKIGHYLKMN |
| | | ITVPEDYINGFIRANNTFLYQLVFDPIKRKLIPLNAYEDDVDPETL |
| | | SYAGQYVDDSIALQIALGNKDINTFEQIDDYNPDTAMPAHSRSRS |
| | | WDDKTCQKSANVSSIWHRNYSPRPESGTVSDAPQLKENPSTVG |
| | | VERVISTKGLNLPRKSSIVKRPRSAELSEDDLLSQYSLSFTKKTKK |
| | | NSSEGNKSLSFSEVFVPDLVNGPTNKKSVSTPPRTRNKFATFLQR |
| | | KNEESGAVVVPGTRSRFFCSSDSTDCVSNKVSIQPLDETAVTDKE |
| | | NNLHESEYGDQEGKRLVDTDVARNSSDDIPNNHIPGDHIPDKAT |
| | | VFTDEESYSFKSSKFTRTISPPTLGTLRSCFSWSGGLGDFSRTPSPS |
| | | PSTALQQFRRKSDSPTSLPENNMSDVSQLKSEESSDDESHPLREG |
| | | ACSSQSQESGEFSLQSSNASKLSQCSSKDSDSEESDCNIKLLDSQS |
| | | DQTSKLCLSHFSKKDTPLRNKVPGLYKSSSADSLSTTKIKPLGPA |
| | | RASGLSKKPASIQKRKHHNAENKPGLQIKLNELWKNFGFKKFRD |
| TrmD | 423 | MWIGIISLFPEMFRAITDYGVTGRAVKNGLLSIQSWSPRDFTHDR |
| (methyl- | | HRTVDDRPYGGGPGMLMMVQPLRDAIHAAKAAAGEGAKVIYL |
| transferase | | SPQGRKLDQAGVSELATNQKLILVCGRYEGIDERVIQTEIDEEWS |
| from E. coli) | | IGDYVLSGGELPAMTLIDSVSRFIPGVLGHEASATEDSFAEGLLD |
| | | CPHYTRPEVLEGMEVPPVLLSGNHAEIRRWRLKQSLGRTWLRRP |
| | | ELLENLALTEEQARLLAEFKTEHAQQQHKHDGMA |
| TrmD | 424 | MWIGVISLFPEMFKAITEFGVTGRAVKHNLLKVECWNPRDFTFD |
| (methyl- | | KHKTVDDRPYGGGPGMLMMVQPLRDAIHTAKAAAGEGAKVIY |
| transferase | | LSPQGRKLDQGGVTELAQNQKLILVCGRYEGIDERLIQTEIDEEW |
| from H. | | SIGDYVLTGGELPAMTLIDAVARFIPGVLGKQASAEEDSFADGLL |
| influenzae) | | DCPHYTRPEVLEGLTVPPVLMSGHHEEIRKWRLKQSLQRTWLR |
| | | RPELLEGLALTDEQRKLLKEAQAEHNS |
| TrmT5 | 425 | MVLWILWRPFGFSGRFLKLESHSITESKSLIPVAWTSLTQMLLEA |
| (methyl- | | PGIFLLGQRKRFSTMPETETHERETELFSPPSDVRGMTKLDRTAF |
| transferase | | KKTVNIPVLKVRKEIVSKLMRSLKRAALQRPGIRRVIEDPEDKES |
| from H. | | RLIMLDPYKIFTHDSFEKAELSVLEQLNVSPQISKYNLELTYEHFK |
| sapiens) | | SEEILRAVLPEGQDVTSGFSRIGHIAHLNLRDHQLSFKHLIGQVMI |
| | | DKNPGITSAVNKINNIDNMYRNFQMEVLSGEQNMMTKVRENNY |
| | | TYEFDFSKVYWNPRLSTEHSRITELLKPGD |
| | | VLFDVFAGVGPFAIPVAKKNCTVFANDLNPESHKWLLYNCKLN |
| | | KVDQKVKVFNLDGKDFLQGPVKEELMQLLGLSKERKPSVHVV |
| | | MNLPAKAIEFLSAFKWLLDGQPCSSEFLPIVHCYSFSKDANPAED |
| | | VRQRAGAVLGISLEACSSVHLVRNVAPNKEMLCITFQIPASVLY |
| | | KNQTRNPENHEDPPLKRQRTAEAFSDEKTQIVSNT |
| Trm5 | 426 | MPLCLKINKKHGEQTRRILIENNLLNKDYKITSEGNYLYLPIKDV |
| (methyl- | | DEDILKSILNIEFELVDKELEEKKIIKKPSFREIISKKYRKEIDEGLIS |
| transferase | | LSYDVVGDLVILQISDEVDEKIRKEIGELAYKLIPCKGVFRRKSEV |
| from M. | | KGEFRVRELEHLAGENRTLTIHKENGYRLWVDIAKVYFSPRLGG |
| jannaschii) | | ERARIMKKVSLNDVVVDMFAGVGPFSIACKNAKKIYAIDINPHAI |
| | | ELLKKNIKLNKLEHKIIPILSDVREVDVKGNRVIMNLPKFAHKFID |
| | | KALDIVEEGGVIHYYTIGKDFDKAIKLFEKKCDCEVLEKRIVKSY |
| | | APREYILALDFKINKK |
| Trm5 (Trm5a | 427 | MSGVKVRREDAKKVLELLKSVGILDGKRKAIRDEKYVIFPVTDT |
| methyl- | | NIAKSLGLEVVDVELPMRPERQIYKNLEDLLPREIFKKLGRLDIV |
| transferase | | GDIAIVSIPDEILSEREVIVSAIRKLYPKVKVIARRGFHSGLYRIRE |
| from P. | | LEVIWGENRLHTIHKENGVLIKVDLSKVFFNPRMKGERYRIAQL |
| abyssi) | | VNDGERILVPFAGVIPYPLVIARFKNVEVYAVEINEFAVKLAEEN |
| | | LELNRDRLKGKIKIIHGDVFEVLPNLPNFDRVVSPTPKGVDALSL |
| | | TLSKAEKFLHYYDFVHESEIERFRERVLEECRRQGKECRVSVRK |
| | | VSDYKPHVYKVCADVEILS |
| Trm5 (Trm5b | 428 | MTLAVKVPLKEGEIVRRRLIELGALDNTYKIKREGNFLLIPVKFP |
| methyl- | | VKGFEVVEAELEQVSRRPNSYREIVNVPQELRRFLPTSFDIIGNIAI |
| transferase | | IEIPEELKGYAKEIGRAIVEVHKNVKAVYMKGSKIEGEYRTRELI |
| from P. | | HIAGENITETIHRENGIRLKLDVAKVYFSPRLATERMRVFKMAQE |
| abyssi) | | GEVVFDMFAGVGPFSILLAKKAELVFACDINPWAIKYLEENIKLN |
| | | KVNNVVPILGDSREIEVKADRIIMNLPKYAHEFLEHAISCINDGG |
| | | VIHYYGFGPEGDPYGWHLERIRELANKFGVKVEVLGKRVIRNYA |
| | | PRQYNIAIDFRVSF |
| Trm5 (Trm5c | 429 | MWSLMHPYLPATVRKWTNLNLSNHSFSLNTMKTKRLSEFFPGV |
| methyl- | | RSYYIVGEIAIITPKRVNVDYNLVAEKIMQAHPKIKAVYLKKKVK |
| transferase | | GELRTNELEFLSGERISSTIYKENGVLFYVDINKVYVNPSLSGDRL |
| from S. | | KNLELVEEGSTVLDAFTGYGAIALNIAHKKRVYVVAGDINIDGL |
| solfataricus) | | YMLKKSLSLNKIKGEMIDIVQYDAHHLPFRDKVFKLSFGDNPTLI |
| | | IDFKEELCRVSENVVFYILCESEDKANSSLGRTSWIKINDYSKNLF |
| | | IFKGLVRC |
| Trm5 (Trm5a | 430 | MFDESKFDVNLKLWALRIPRELCKSASRILNGYMLNMPRIKPITE |
| methyl- | | DPTCEKTRLVILSESVKNADLSEIPEEKLNQLKKLSELEVVPHSVT |
| transferase | | LGYSYWSADHLLKQILPDGLDIPSSFETIGHIAHLNLHDELLPFKD |
| from A. | | VIAKVIYDKNYPRIKTIVNKVGTISNEFRVPKFEVLAGENGMETE |
| thaliana) | | VKQYGARFKLDYGLVYWNSRLEHEHMRLSSLFKPGETVCDMF |
| | | AGIGPFAIPAAQKGCFVYANDLNPDSVRYLKINAKFNKVDDLIC |
| | | VHNMDARKFFSHLMAVSTCEDNLQSVADNDKTKEAAVSRGGE |
| | | TNSSGEEIRESNASINEPLGANKKPSGTTKTENGVGKDCKSIEGH |
| | | ANKRLRQTLLPIAKPWEHIDHVIMNLPASALQFLDSFSNVIQKKY |
| | | WKGPLPLIHCYCFIRASETTEFIIAEAETALKFHIEDPVFHKVRDV |
| | | APNKAMFCLSFRLPEACLKQEE |
| Trm5 | 431 | MKIALPVFQKFNRLISSCKMSGVFPYNPPVNRQMRELDRSFFITK |
| (methyl- | | IPMCAVKFPEPKNISVFSKNFKNCILRVPRIPHVVKLNSSKPKDEL |
| transferase | | TSVQNKKLKTADGNNTPVTKGVLLHESIHSVEDAYGKLPEDAL |
| from S. | | AFLKENSAEIVPHEYVLDYDFWKAEEILRAVLPEQFLEEVPTGFT |
| cerevisiae) | | ITGHIAHLNLRTEFKPFDSLIGQVILDKNNKIECVVDKVSSIATQF |
| | | RTFPMKVIAGKSDSLVVEQKESNCTFKFDFSKVYWNSRLHTEHE |
| | | RLVKQYFQPGQVVCDVFAGVGPFAVPAGKKDVIVLANDLNPES |
| | | YKYLKENIALNKVAKTVKSFNMDGADFIRQSPQLLQQWIQDEE |
| | | GGKITIPLPLKKRHRSQQHNDQQPPQPRTKELIIPSHISHYVMNLP |
| | | DSAISFLGNFRGIFAAHTKGATDTIQMPWVHVHCFEKYPPGDQV |
| | | TEDELHARVHARIIAALKVTADDLPLNAVSLHLVRKVAPTKPMY |
| | | CASFQLPANV |
| TrmT5 | 432 | MRIVWKLFGFSRRLLQVEWCHPSESILLFTLVPRLRKAPSVFLLG |
| (methyl- | | QRQGLSTMPEIEASVRDSELFSPPSDVRGMRELDRTAFKKTVSIP |
| transferase | | VLKARKEVVNRLMRALRRVALQRPGIKRVIEDPKDEDSRLIMLD |
| from M. | | PYRMLTADSFDKAELGVLKELDVSPQLSQYNLELTYENFKSEEIL |
| musculus) | | KAVLPEGQDVTSGFSRVGHIAHLNLRDHQLPFKHLIGQVMVDK |
| | | NPGITSAVNKTSNIDNTYRNFQMEVLCGEENMLTKVRENNYTYE |
| | | FDFSKVYWNPRLSTEHGRITELLNPGDVLFDVFAGVGPFAIPAAR |
| | | KNCTVFANDLNPESHKWLLHNCKLNKVDQKVKVFNMDGKDFI |
| | | QGPVREELMLRLGLSAEAKPSVHIVMNLPAKAIEFLSVFRSLLDG |
| | | QPCSTELLPTVHCYCFSKDSDPAKDVRQQAEAVLGVSLETSSSV |
| | | HLVRNVAPNKEMLCITFQIPTATLYRNQSLSLQNDQEPPLKRQK |
| | | TGDPFSGEPQIASDS |
| Trm10 | 433 | MSNDEINQNEEKVKRTPPLPPVPEGMSKKQWKKMCKRQRWEE |
| (methyl- | | NKAKYNAERRVKKKRLRHERSAKIQEYIDRGEEVPQELIREPRIN |
| transferase | | VNQTDSGIEIILDCSFDELMNDKEIVSLSNQVTRAYSANRRANHF |
| from S. | | AEIKVAPFDKRLKQRFETTLKNTNYENWNHFKFLPDDKIMFGDE |
| cerevisiae) | | HISKDKIVYLTADTEEKLEKLEPGMRYIVGGIVDKNRYKELCLK |
| | | KAQKMGIPTRRLPIDEYINLEGRRVLTTTHVVQLMLKYFDDHN |
| | | WKNAFESVLPPRKLDAEAKSASSSPAPKDT |
| Trm10 | 434 | MENKDALDIGKDDTNTSEADVSKNETQEQPVLSKSALKRLKRQ |
| (methyl | | QEWDAGREKRAEMRREKKRLRKEERKRKIEAGEVVKSQKKRIR |
| transferase | | LGKVVPSSIRIVLDCAFDDLMNDKEINSLCQQVTRCHSANRTAL |
| from S. | | HPVELFATNFGGRLKTRQDFVLKGQQNNWKRYNPTTKSYLEEF |
| pombe) | | ESQKEKLVYLSADSDNTITELDEDKIYIIGAIVDKNRYKNLCQNK |
| | | ASEQGIKTAKLPIDEYIKITDRKILTVNQVFEILSLWLEYRDWEKA |
| | | FMEVIPKRKGILLKSDESFDVSEDTRSQSNQSDSELEKEN |
| TrmT10 | 435 | MSSEMLPAFIETSNVDKKQGINEDQEESQKPRLGEGCEPISKRQM |
| (TrmT10A | | KKLIKQKQWEEQRELRKQKRKEKRKRKKLERQCQMEPNSDGH |
| methyl- | | DRKRVRRDVVHSTLRLIIDCSFDHLMVLKDIKKLHKQIQRCYAE |
| transferase | | NRRALHPVQFYLTSHGGQLKKNMDENDKGWVNWKDIHIKPEH |
| from H. | | YSELIKKEDLIYLTSDSPNILKELDESKAYVIGGLVDHNHHKGLT |
| sapiens) | | YKQASDYGINHAQLPLGNFVKMNSRKVLAVNHVFEIILEYLETR |
| | | DWQEAFFTILPQRKGAVPTDKACESASHDNQSVRMEEGGSDSD |
| | | SSEEEYSRNELDSPHEEKQDKENHTESTVNSLPH |
| TRrmT10 | 436 | MDWKLEGSTQKVESPVLQGQEGILEETGEDGLPEGFQLLQIDAE |
| (TRrmT10B | | GECQEGEILATGSTAWCSKNVQRKQRHWEKIVAAKKSKRKQEK |
| methyl- | | ERRKANRAENPGICPQHSKRFLRALTKDKLLEAKHSGPRLCIDLS |
| transferase | | MTHYMSKKELSRLAGQIRRLYGSNKKADRPFWICLTGFTTDSPL |
| from H. | | YEECVRMNDGFSSYLLDITEEDCFSLFPLETLVYLTPDSEHALED |
| sapiens) | | VDLNKVYILGGLVDESIQKKVTFQKAREYSVKTARLPIQEYMVR |
| | | NQNGKNYHSEILAINQVFDILSTYLETHNWPEALKKGVSSGKGYI |
| | | LRNSVE |
| TrmT10 | 437 | MAAFLKMSVSVNFFRPFTRFLVPFTLHRKRNNLTILQRYMSSKIP |
| (TrmT10C | | AVTYPKNESTPPSEELELDKWKTTMKSSVQEECVSTISSSKDEDP |
| methyl- | | LAATREFIEMWRLLGREVPEHITEEELKTLMECVSNTAKKKYLK |
| transferase | | YLYTKEKVKKARQIKKEMKAAAREEAKNIKLLETTEEDKQKNF |
| from H. | | LFLRLWDRNMDIAMGWKGAQAMQFGQPLVFDMAYENYMKRK |
| sapiens) | | ELQNTVSQLLESEGWNRRNVDPFHIYFCNLKIDGALHRELVKRY |
| | | QEKWDKLLLTSTEKSHVDLFPKDSIIYLTADSPNVMTTFRHDKV |
| | | YVIGSFVDKSMQPGTSLAKAKRLNLATECLPLDKYLQWEIGNKN |
| | | LTLDQMIRILLCLKNNGNWQEALQFVPKRKHTGFLEISQHSQEFI |
| | | NRLKKAKT |

In some embodiments, a fusion partner described herein comprises any one of the amino acid sequences set forth in TABLE 2 and TABLE 3. In some embodiments, effector proteins described herein comprise an amino acid sequence that is at least about 6500, at least about 70%, at least about 75%, at least about 80%, at least about 85%, at least about 90%, at least about 95%, at least about 97%, at least about 98%, at least about 99%, or about 100% identical to any one of the sequences recited in TABLE 1 and further comprises one or more of the sequences set forth in TABLE 2 and TABLE 3.

### Base Editors

In some embodiments, fusion partners modify a nucleobase of a target nucleic acid. Fusion proteins comprising such fusion partners and a Cas effector protein may be referred to as base editors. In some embodiments, base editors modify a sequence of a target nucleic acid. In some embodiments, base editors provide a nucleobase change in a DNA molecule. In some embodiments, the nucleobase change in the DNA molecule is selected from: an adenine (A) to guanine (G); cytosine (C) to thymine (T); and cytosine (C) to guanine (G). In some embodiments, base editors provide a nucleobase change in an RNA molecule. In some embodiments, the nucleobase change in the RNA molecule is selected from: adenine (A) to guanine (G); uracil (U) to cytosine (C); cytosine (C) to guanine (G); and guanine (G) to adenine (A). In some embodiments, the fusion partner is a deaminase, e.g., ADAR1/2.

Some base editors modify a nucleobase of on a single strand of DNA. In some embodiments, base editors modify a nucleobase on both strands of dsDNA. In some embodiments, upon binding to its target locus in DNA, base pairing between the guide RNA and target DNA strand leads to displacement of a small segment of single-stranded DNA in an “R-loop”. In some embodiments, DNA bases within the R-loop are modified by the deaminase enzyme. In some embodiments, DNA base editors for improved efficiency in eukaryotic cells comprise a catalytically inactive effector protein that may generate a nick in the non-edited DNA strand, inducing repair of the non-edited strand using the edited strand as a template.

Some base editors modify a nucleobase of an RNA. In some embodiments, RNA base editors comprise an adenosine deaminase. In some embodiments, ADAR proteins bind to RNAs and alter their sequence by changing an adenosine into an inosine. In some embodiments, RNA base editors comprise a Cas effector protein that is activated by or binds RNA. Non-limiting examples of Cas effector proteins that are activated by or bind RNA are Cas13 proteins.

In some embodiments, base editors are used to treat a subject having or a subject suspected of having a disease related to a gene of interest. In some embodiments, base editors are useful for treating a disease or a disorder caused by a point mutation in a gene of interest. In some embodiments, compositions comprise a base editor and a guide nucleic acid, wherein the guide nucleic acid directs the base editor to a sequence in a target gene. The target gene may be associated with a disease. In some embodiments, the guide nucleic acid directs that base editor to or near a mutation in the sequence of a target gene. The mutation may be the deletion of one more nucleotides. The mutation may be the addition of one or more nucleotides. The mutation may be the substitution of one or more nucleotides. The mutation may be the insertion, deletion or substitution of a single nucleotide, also referred to as a point mutation. The point mutation may be a SNP. The mutation may be associated with a disease. In some embodiments, the guide nucleic acid directs the base editor to bind a target sequence within the target nucleic acid that is within 0, 1, 2, 3, 4, 5, 6, 7, 8, 9, 10, 11, 12, 13, 14, 15, 16, 17, 18, 19, or 20 nucleotides of the mutation. In some embodiments, the guide nucleic acid comprises a sequence that is identical, complementary or reverse complementary to a target sequence of a target nucleic acid that comprises the mutation. In some embodiments, the guide nucleic acid comprises a sequence that is identical, complementary or reverse complementary to a target sequence of a target nucleic acid that is within 1, 2, 3, 4, 5, 6, 7, 8, 9, 10, 11, 12, 13, 14, 15, 16, 17, 18, 19, or 20 nucleotides of the mutation.

Some base editors modify a nucleobase of an RNA. In some embodiments, RNA base editors comprise an adenosine deaminase. In some embodiments, ADAR proteins bind to RNAs and alter their sequence by changing an adenosine into an inosine. In some embodiments, RNA base editors comprise a Cas effector protein that is activated by or binds RNA. Non-limiting examples of Cas effector proteins that are activated by or bind RNA are Cas13 proteins.

In some embodiments, base editors are used to treat a subject having or a subject suspected of having a disease related to a gene of interest. In some embodiments, base editors are useful for treating a disease or a disorder caused by a point mutation in a gene of interest. In some embodiments, compositions comprise a base editor and a guide nucleic acid, wherein the guide nucleic acid directs the base editor to a sequence in a target gene.

In some embodiments, a base editor may be a base editing enzyme. Accordingly, in some embodiments, fusion partners comprise a base editing enzyme. In some embodiments, the base editing enzyme modifies the nucleobase of a deoxyribonucleotide. In some embodiments, the base editing enzyme modifies the nucleobase of a ribonucleotide. A base editing enzyme that converts a cytosine to a guanine or thymine may be referred to as a cytosine base editing enzyme. A base editing enzyme that converts an adenine to a to a guanine may be referred to as an adenine base editing enzyme. In some embodiments, the base editing enzyme comprises a deaminase enzyme. In some embodiments, the deaminase functions as a monomer. In some embodiments, the deaminase functions as heterodimer with an additional protein. In some embodiments, base editors comprise a DNA glycosylase inhibitor. In some embodiments, base editors comprise a uracil glycosylase inhibitor (UGI) or uracil N-glycosylase (UNG). In some embodiments, base editors do not comprise a UGI. In some embodiments, base editors do not comprise a UNG. In some embodiments, base editors do not comprise a functional fragment of a UGI. A functional fragment of a UGI is a fragment of a UGI that is capable of excising a uracil residue from DNA by cleaving an N-glycosydic bond.

In some embodiments, the base editor is a cytidine deaminase base editor generated by ancestral sequence reconstruction as described in WO2019226953, which is hereby incorporated by reference in its entirety.

Exemplary deaminase domains are described WO 2018027078 and WO2017070632, and each are hereby incorporated in its entirety by reference. Also, additional exemplary deaminase domains are described in Komor et al., Nature, 533, 420-424 (2016); Gaudelli et al., Nature, 551, 464-471 (2017); Komor et al., Science Advances, 3:eaao4774 (2017), and Rees et al., Nat Rev Genet. 2018 December; 19(12):770-788. doi: 10.1038/s41576-018-0059-1, which are hereby incorporated by reference in their entirety.

In some embodiments, the base editor is a cytosine base editor (CBE). In general, a CBE comprises a cytosine base editing enzyme and a catalytically inactive effector protein. In some embodiments, the catalytically inactive effector protein is a catalytically inactive variant of a Cas effector protein described herein. The CBE may convert a cytosine to a thymine. In some embodiments, the base editor is an adenine base editor (ABE). In general, an ABE comprises an adenine base editing enzyme and a catalytically inactive effector protein. In some embodiments, the catalytically inactive effector protein is a catalytically inactive variant of a Cas effector protein described herein. The ABE generally converts an adenine to a guanine. In some embodiments, the base editor is a cytosine to guanine base editor (CGBE). In general, a CGBE converts a cytosine to a guanine.

In some embodiments, the base editor is a CBE. In some embodiments, the cytosine base editing enzyme is a cytidine deaminase. In some embodiments, the cytosine deaminase is an APOBEC1 cytosine deaminase, which accept ssDNA as a substrate but is incapable of cleaving dsDNA, fused to a catalytically inactive effector protein. In some embodiments, when bound to its cognate DNA, the catalytically inactive effector protein performs local denaturation of the DNA duplex to generate an R-loop in which the DNA strand not paired with the guide RNA exists as a disordered single-stranded bubble. In some embodiments, the catalytically inactive effector protein generated ssDNA R-loop enables the CBE to perform efficient and localized cytosine deamination in vitro. In some examples, deamination activity is exhibited in a window of about 4 to about 10 base pairs. In some embodiments, fusion to the catalytically inactive effector protein presents the target site to APOBEC1 in high effective molarity, enabling the CBE to deaminate cytosines located in a variety of different sequence motifs, with differing efficacies. In some embodiments, the CBE is capable of mediating RNA-programmed deamination of target cytosines in vitro. In some embodiments, the CBE is capable of mediating RNA-programmed deamination of target cytosines in vivo. In some embodiments, the cytosine base editing enzyme is a cytosine base editing enzyme described by Koblan et al. (2018) Nature Biotechnology 36:848-846; Komor et al. (2016) Nature 533:420-424; Koblan et al. (2021) “Efficient C•G-to-G•C base editors developed using CRISPRi screens, target-library analysis, and machine learning,” Nature Biotechnology; Kurt et al. (2021) Nature Biotechnology 39:41-46; Zhao et al. (2021) Nature Biotechnology 39:35-40; and Chen et al. (2021) Nature Communications 12:1384, all incorporated herein by reference.

In some embodiments, CBEs comprise a uracil glycosylase inhibitor (UGI) or uracil N-glycosylase (UNG). In some embodiments, base excision repair (BER) of U•G in DNA is initiated by a UNG, which recognizes the U•G mismatch and cleaves the glyosidic bond between uracil and the deoxyribose backbone of DNA. In some embodiments, BER results in the reversion of the U•G intermediate created by the first CBE back to a C•G base pair. In some embodiments, UNG may be inhibited by fusion of uracil DNA glycosylase inhibitor (UGI), in some embodiments, a small protein from bacteriophage PBS, to the C-terminus of the CBE. In some embodiments, UGI is a DNA mimic that potently inhibits both human and bacterial UNG. In some embodiments, a UGI inhibitor is any protein or polypeptide that inhibits UNG. In some embodiments, the CBE mediates efficient base editing in bacterial cells and moderately efficient editing in mammalian cells, enabling conversion of a C•G base pair to a T•A base pair through a U•G intermediate. In some embodiments, the CBE is modified to increase base editing efficiency while editing more than one strand of DNA.

In some embodiments, the CBE nicks the non-edited DNA strand. In some embodiments, the non-edited DNA strand nicked by the CBE biases cellular repair of the U•G mismatch to favor a U•A outcome, elevating base editing efficiency. In some embodiments, the APOBEC1-nickase-UGI fusion efficiently edits in mammalian cells, while minimizing frequency of non-target indels.

In some embodiments, the cytidine deaminase is selected from APOBECI, APOBEC2, APOBEC3C, APOBEC3D, APOBEC3F, APOBEC3G, APOBEC3H, APOBEC4, APOBEC3A, BE1 (APOBEC1-XTEN-dCas9), BE2 (APOBEC1-XTEN-dCas9-UGI), BE3 (APOBEC1-XTEN-dCas9(A840H)-UGI), BE3-Gam, saBE3, saBE4-Gam, BE4, BE4-Gam, saBE4, or saBE4-Gam as described in WO2021163587, WO202108746, WO2021062227, and WO2020123887, which are incorporated herein by reference in their entirety.

In some embodiments, the fusion protein further comprises a non-protein uracil-DNA glcosylase inhibitor (npUGI). In some embodiments, the npUGI is selected from a group of small molecule inhibitors of uracil-DNA glycosylase (UDG), or a nucleic acid inhibitor of UDG. In some embodiments, the non-protein uracil-DNA glcosylase inhibitor (npUGI) is a small molecule derived from uracil. Examples of small molecule non-protein uracil-DNA glcosylase inhibitors, fusion proteins, and Cas-CRISPR systems comprising base editing activity are described in WO202108746, which is incorporated by reference in its entirety.

In some embodiments, the fusion partner is a deaminase, e.g., ADAR1/2, ADAR-2, or AID. In some embodiments, the base editor is an ABE. In some embodiments, the adenine base editing enzyme of the ABE is an adenosine deaminase. In some embodiments, the adenine base editing enzyme is selected from ABE8e, ABE8.20m, APOBEC3A, Anc APOBEC, and BtAPOBEC2. In some embodiments, the ABE base editor is an ABE7 base editor. In some embodiments, the deaminase or enzyme with deaminase activity is selected from ABE8.1m, ABE8.2m, ABE8.3m, ABE8.4m, ABE8.5m, ABE8.6m, ABE8.7m, ABE8.8m, ABE8.9m, ABE8.10m, ABE8.11m, ABE8.12m, ABE8.13m, ABE8.14m, ABE8.15m, ABE8.16m, ABE8.17m, ABE8.18m, ABE8.19m, ABE8.20m, ABE8.21m, ABE8.22m, ABE8.23m, ABE8.24m, ABE8.1d, ABE8.2d, ABE8.3d, ABE8.4d, ABE8.5d, ABE8.6d, ABE8.7d, ABE8.8d, ABE8.9d, ABE8.10d, ABE8.11d, ABE8.12d, ABE8.13d, ABE8.14d, ABE8.15d, ABE8.16d, ABE8.17d, ABE8.18d, ABE8.19d, ABE8.20d, ABE8.21d, ABE8.22d, ABE8.23d, or ABE8.24d. In some embodiments, the adenine base editing enzyme is ABE8.1d. In some embodiments, the adenosine base editor is ABE9. Exemplary deaminases are described in US20210198330, WO2021041945, WO2021050571A1, and WO2020123887, all of which are incorporated herein by reference in their entirety. Sequences of a selection of these enzymes are provided in TABLE 2. In some embodiments, the adenine base editing enzyme is an adenine base editing enzyme described in Chu et al., (2021) The CRISPR Journal 4:2:169-177, incorporated herein by reference. In some embodiments, the adenine deaminase is an adenine deaminase described by Koblan et al. (2018) Nature Biotechnology 36:848-846, incorporated herein by reference. In some embodiments, the adenine base editing enzyme is an adenine base editing enzyme described by Tran et al. (2020) Nature Communications 11:4871. Additional examples of deaminase domains are also described in WO2018027078 and WO2017070632, which are hereby incorporated by reference in their entirety.

In some embodiments, an ABE converts an A•T base pair to a G•C base pair. In some embodiments, the ABE converts a target A•T base pair to G•C in vivo. In some embodiments, the ABE converts a target A•T base pair to G•C in vitro. In some embodiments, ABEs provided herein reverse spontaneous cytosine deamination, which has been linked to pathogenic point mutations. In some embodiments, ABEs provided herein enable correction of pathogenic SNPs (˜47% of disease-associated point mutations). In some embodiments, the adenine comprises exocyclic amine that has been deaminated (e.g., resulting in altering its base pairing preferences). In some embodiments, deamination of adenosine yields inosine. In some embodiments, inosine exhibits the base-pairing preference of guanine in the context of a polymerase active site, although inosine in the third position of a tRNA anticodon is capable of pairing with A, U, or C in mRNA during translation. In some embodiments, an ABE comprises an engineered adenosine deaminase enzyme capable of acting on ssDNA.

In some embodiments, a base editor comprises an adenosine deaminase variant that differs from a naturally occurring deaminase. Relative to the naturally occurring deaminase, the adenosine deaminase variant may comprise a V82S alteration, a T166R alteration, or a combination thereof. In some embodiments, the adenosine deaminase variant comprises at least one of the following alterations relative to a naturally occurring adenosine deaminase: Y147T, Y147R, Q154S, Y123H, and Q154R., which are incorporated herein by reference in their entirety.

In some embodiments, a base editor comprises a deaminase dimer. In some embodiments, a base editor is a deaminase dimer further comprising a base editing enzyme and an adenine deaminase (e.g., TadA).

TadA comprises or consists of at least a portion of the sequence:

(SEQ ID NO: 560)
SEVEFSHEYWMRHALTLAKRAWDEREVPVGAVLVHNNRVIGEGWNRPIG
RHDPTAHAEIMALRQGGLVMQNYRLIDATLYVTLEPCVMCAGAMIHSRI
GRVVFGARDAKTGAAGSLMDVLHHPGMNHRVEITEGILADECAALLSDF
FRMRRQEIKAQKKAQSSTD.

In some embodiments, the adenosine deaminase is a TadA monomer (e.g., Tad*7.10, TadA*8 or TadA*9). In some embodiments, the adenosine deaminase is a TadA*8 variant. Such a TadA*8 variant includes TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24 as described in WO2021163587 and WO2021050571, which are each hereby incorporated by reference in its entiry.

In some embodiments, a base editor is a deaminase dimer comprising a base editing enzyme fused to TadA via a linker. In some embodiments the linker comprises or consists of at least a portion of the sequence:

SGGSSGGSSGSETPGTSESATPESSGGSSGGS (SEQ ID NO: 561). In some embodiments, the amino acid sequence of the linker is 70%, 75%, 80%, 85%, 90%, or 95% identical to SGGSSGGSSGSETPGTSESATPESSGGSSGGS (SEQ ID NO: 561).

In some embodiments, the amino terminus of the fusion partner protein is linked to the carboxy terminus of the effector protein via the linker. In some embodiments, the carboxy terminus of the fusion partner protein is linked to the amino terminus of the effector protein via the linker.

In some embodiments, the base editing enzyme is fused to TadA at the N-terminus. In some embodiments, the base editing enzyme is fused to TadA at the C-terminus. In some embodiments, the base editing enzyme is a deaminase dimer comprising an ABE. In some embodiments, the deaminase dimer comprises an adenosine deaminase. In some embodiments, the deaminase dimer comprises TadA fused to an adenine base editing enzyme selected from ABE8e, ABE8.20m, APOBEC3A, Anc APOBEC, and BtAPOBEC2. In some embodiments TadA is fused to ABE8e or a variant thereof. In some embodiments TadA is fused to ABE8e or a variant thereof at the amino-terminus (ABE8e-TadA). In some embodiments, TadA is fused to ABE8e or a variant thereof at the carboxy terminus (ABE8e-TadA).

### Prime Editing Enzyme

In some embodiments, a fusion partner can comprise a prime editing enzyme. In some embodiments, a prime editing enzyme comprises a reverse transcriptase. A non-limiting example of a reverse transcriptase is an M-MLV RT enzyme and variants thereof having polymerase activity. In some embodiments, the M-HLV RT enzyme comprises at least one mutation selected from D200N, L603W, T330P, T306K, and W313F relative to wildtype M-MLV RT enzyme. A prime editing enzyme may require a pegRNA and a single guide RNA to catalyze the modification. In some embodiments, the target nucleic acid is a dsDNA molecule. In some embodiments, the pegRNA comprises a guide RNA comprising a first region that is bound by the effector protein, and a second region comprising a spacer sequence that is complementary to a target sequence of the dsDNA molecule; a template RNA comprising a primer binding sequence that hybridizes to a primer sequence of the dsDNA molecule that is formed when target nucleic acid is cleaved, and a template sequence that is complementary to at least a portion of the target sequence of the dsDNA molecule with the exception of at least one nucleotide. In some embodiments, the spacer sequence is complementary to the target sequence on a target strand of the dsDNA molecule. In some embodiments, the spacer sequence is complementary to the target sequence on a non-target strand of the dsDNA molecule. In some embodiments, the primer binding sequence hybridizes to a primer sequence on the non-target strand of the dsDNA molecule. In some embodiments, the primer binding sequence hybridizes to a primer sequence on the target strand of the dsDNA molecule. In some embodiments, the target strand is cleaved. In some embodiments, the non-target strand is cleaved.

### CRISPR Fusion Partners

In some embodiments, fusion partners include, but are not limited to, a protein that directly and/or indirectly provides for increased or decreased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.). In some embodiments, fusion partners that increase or decrease transcription include a transcription activator domain or a transcription repressor domain, respectively.

### CRISPRi Fusion Partners

In some embodiments, fusions partners inhibit or reduce expression of a target nucleic acid. In some embodiments, such fusion partners include, but are not limited to, a protein that directly and/or indirectly provides for decreased transcription and/or translation of a target nucleic acid (e.g., a small molecule/drug-responsive transcription and/or translation regulator, a translation-regulating protein, etc.). Fusion proteins comprising such fusion partners and a Cas effector protein may be referred to as CRISPRi fusions. In some embodiments, fusion partners reduce expression of the target nucleic acid relative to its expression in the absence of the fusion effector protein. Relative expression, including transcription and RNA levels, may be assessed, quantified, and compared, e.g., by RT-qPCR. In some embodiments, fusion partners may comprise a transcriptional repressor. Transcriptional repressors may inhibit transcription via: recruitment of other transcription factor proteins; modification of target DNA such as methylation; recruitment of a DNA modifier; modulation of histones associated with target DNA; recruitment of a histone modifier such as those that modify acetylation and/or methylation of histones; or a combination thereof. Non-limiting examples of fusion partners that decrease or inhibit transcription include, but are not limited to: transcriptional repressors such as the Krüppel associated box (KRAB or SKD); KOX1 repression domain; the Mad mSIN3 interaction domain (SID); the ERF repressor domain (ERD), the SRDX repression domain (e.g., for repression in plants); histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID1C/SMCX, JARID1D/SMCY; histone lysine deacetylases such as HDAC1, HDAC2, HDAC3, HDAC8, HDAC4, HDAC5, HDAC7, HDAC9, SIRT1, SIRT2, HDAC11; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants); and periphery recruitment elements such as Lamin A, and Lamin B; and functional domains thereof. Other non-limiting examples of suitable fusion partners include: proteins and protein domains responsible for repressing translation (e.g., Ago2 and Ago4); proteins and protein domains responsible for repression of RNA splicing (e.g., PTB, Sam68, and hnRNP A1); proteins and protein domains responsible for reducing the efficiency of transcription (e.g., FUS (TLS)).

### CRISPRa Fusion Partners

In some embodiments, fusion partners activate or increase expression of a target nucleic acid. In some embodiments, such fusion partners include, but are not limited to, a protein that directly and/or indirectly provides for increased transcription and/or translation of a target nucleic acid (e.g., a transcription activator or a fragment thereof, a protein or fragment thereof that recruits a transcription activator, a small molecule/drug-responsive transcription and/or translation regulator, a translation-regulating protein, etc.). Fusion proteins comprising such fusion partners and a Cas effector protein may be referred to as CRISPRa fusions. In some embodiments, fusion partners increase expression of the target nucleic acid relative to its expression in the absence of the fusion effector protein. Relative expression, including transcription and RNA levels, may be assessed, quantified, and compared, e.g., by RT-qPCR. In some embodiments, fusion partners comprise a transcriptional activator. Transcriptional activators may promote transcription via: recruitment of other transcription factor proteins; modification of target DNA such as demethylation; recruitment of a DNA modifier; modulation of histones associated with target DNA; recruitment of a histone modifier such as those that modify acetylation and/or methylation of histones; or a combination thereof. Non-limiting examples of fusion partners that activate or increase transcription include, but are not limited to: transcriptional activators such as VP16, VP64, VP48, VP160, p65 subdomain (e.g., from NFkB), an activation domain of EDLL and/or TAL activation domain (e.g., for activity in plants); histone lysine methyltransferases such as SET1A, SET1B, MLL1 to 5, ASH1, SYMD2, NSD1; histone lysine demethylases such as JHDM2a/b, UTX, JMJD3; histone acetyltransferases such as GCN5, PCAF, CBP, p300, TAF1, TIP60/PLIP, MOZ/MYST3, MORF/MYST4, SRC1, ACTR, P160, CLOCK; and DNA demethylases such as Ten-Eleven Translocation (TET) dioxygenase 1 (TET1CD), TET1, DME, DML1, DML2, and ROS1; and functional domains thereof.

RNA splicing

In some embodiments, fusion partners comprise an RNA splicing factor. The RNA splicing factor may be used (in whole or as fragments thereof) for modular organization, with separate sequence-specific RNA binding modules and splicing effector domains. Non-limiting examples of RNA splicing factors include members of the Serine/Arginine-rich (SR) protein family contain N-terminal RNA recognition motifs (RRMs) that bind to exonic splicing enhancers (ESEs) in pre-mRNAs and C-terminal RS domains that promote exon inclusion. As another example, the hnRNP protein hnRNP A1 binds to exonic splicing silencers (ESSs) through its RRM domains and inhibits exon inclusion through a C-terminal Glycine-rich domain. Some splicing factors may regulate alternative use of splice site (ss) by binding to regulatory sequences between the two alternative sites. For example, ASF/SF2 may recognize ESEs and promote the use of intron proximal sites, whereas hnRNP A1 may bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. The long splicing isoform Bcl-xL is a potent apoptosis inhibitor expressed in long-lived postmitotic cells and is up-regulated in many cancer cells, protecting cells against apoptotic signals. The short isoform Bcl-xS is a pro-apoptotic isoform and expressed at high levels in cells with a high turnover rate (e.g., developing lymphocytes). The ratio of the two Bcl-x splicing isoforms is regulated by multiple c\acute{ω}-elements that are located in either the core exon region or the exon extension region (i.e., between the two alternative 5′ splice sites). For more examples, see WO2010075303, which is hereby incorporated by reference in its entirety.

In some embodiments, fusion partners comprise a protein or protein domain responsible for repression of RNA splicing. Non-limiting examples of proteins and protein domains responsible for repression of RNA splicing include PTB, Sam68, and hnRNP A1 as described in WO2021041846, which is hereby incorporated by reference in its entirety.

In some embodiments, fusion partners comprise proteins that are boundary elements, meaning that they are proteins or fragments thereof that provide periphery recruitment. Non-limiting examples include CTCF, protein docking elements such as FKBP/FRB, and Pill/Aby1 as described in WO2021041846, which is hereby incorporated by reference in its entirety.

### Recombinases

In some embodiments, the fusion partners comprise a recombinase domain. In some embodiments, the recombinase is a site-specific recombinase. In some embodiments, the recombinase is a tyrosine recombinase. Non-limiting examples of serine recombinases include, but are not limited to, Cre, Flp, and lambda integrase. In some embodiments, the recombinase is a serine recombinase. Non-limiting examples of serine recombinases include, but are not limited to, gamma-delta resolvase, Tn3 resolvase, Sin resolvase, Gin invertase, Hin invertase, Tn5044 resolvase, IS607 transposase, and IS607 integrase. In some embodiments, the site-specific recombinase is an integrase. Non-limiting examples of integrases include, but are not limited to:Bxb1, wBeta, BL3, phiR4, A118, TG1, MR11, phi370, SPBc, TP901-1, phiRV, FC1, K38, phiBT1, and phiC31. Further discussion and examples of suitable recombinase fusion partners are described in U.S. Pat. No. 10,975,392, which is incorporated herein by reference in its entirety.

In some embodiments, the fusion protein comprises a linker that links the recombinase domain to the Cas-CRISPR domain of the effector protein. In some embodiments, the linker is The-Ser.

### DNA Alkylating Fusion Partners

Disclosed herein is a DNA alkylating fusion partner. In some embodiments, a fusion effector protein is a DNA alkylating fusion protein comprising: (a) an DNA alkylating fusion partner as described herein; and (b) an effector protein as described herein, wherein the DNA alkylating fusion partner is linked to the effector protein via a linker. In some embodiments, the DNA alkylating fusion protein further comprises a repair inhibitor fusion partner as described herein.

In some embodiments, the DNA alkylating fusion protein can also be referred to as an engineered enzymatic effector protein. In some embodiments, the DNA alkylating fusion partner, upon contact with a double stranded DNA molecule alkylates the double stranded DNA molecule. In some embodiments, the DNA alkylating fusion partner alkylates the guanine or thymine of the double stranded DNA molecule site specifically. In some embodiments, the DNA alkylating fusion partner, upon contact with double stranded DNA molecule, performs O-alkylation at O⁶-guanine in the non-target strand of the double stranded DNA molecule. In some embodiments, the DNA alkylating fusion partner, upon contact with double stranded DNA molecule, performs O-alkylation at O⁴-thymine in the non-target strand of the double stranded DNA molecule. In some embodiments, the DNA alkylating fusion partner, upon contact with double stranded DNA molecule, performs N-alkylation at N¹-guanine in the non-target strand of the double stranded DNA molecule. In some embodiments, the O-alkylation or N-alkylation is an O— methylation or N-methylation, respectively. In some embodiments, the DNA alkylating fusion partner performs: (a) O-alkylation at O⁶-guanine present in the non-target strand of the double stranded DNA molecule; (b) O-alkylation at O⁴-thymine present in the non-target strand of the double stranded DNA molecule; or (c) N-alkylation at N¹-guanine present in the non-target strand of the double stranded DNA molecule. In some embodiments, the DNA alkylating fusion partner is selected from an engineered DNMT3a, DNMT3b, DNMT1, DAM, and a functional portion thereof. In some embodiments, the DNA alkylating fusion partner is an engineered RNA methyl transferase. In some embodiments, the engineered RNA methyl transferase is selected from an engineered Trm5, TrmD, Trm10, RsmE, BMT5, BMT6, and a functional portion thereof.

In some embodiments, a DNA alkylating fusion partner is a methyl transferase fusion partner. In some embodiments, the methyl transferase fusion partner is also referred as an engineered methyl transferase enzyme. In some embodiments, the methyl transferase fusion partner, upon contact with a single strand of the double stranded DNA molecule, methylates cytosine residues of the single strand of the double stranded DNA molecule, thereby producing methyl cytosine. In some embodiments, the methyl transferase fusion partner, upon contact with a single strand of a double stranded DNA molecule, methylates cytosine residues of the single strand of the double stranded DNA molecule faster than cytosine residues of an otherwise comparable double stranded DNA molecule when contacted with the otherwise comparable double stranded DNA molecule. In some embodiments, the methyl transferase fusion partner, upon contact with a target single stranded DNA molecule, methylates cytosine residues of the target single stranded DNA molecule about 2-fold to about 10-fold faster than cytosine residues in an otherwise comparable double stranded DNA molecule when contacted with the otherwise comparable double stranded DNA molecule. In some embodiments, the methyl transferase fusion partner, upon contact with the target single stranded DNA molecule, methylates cytosine residues of the target single stranded DNA molecule at least 4-fold faster than cytosine residues of an otherwise comparable double stranded DNA molecule when contacted with the otherwise comparable double stranded DNA molecule.

In some embodiments, the methyl transferase fusion partner comprises DNMT3a, DNMT3b, DNMT1, DAM, Trm5, TrmD, Trm10, TrmT10, TrmT5, RsmE, BMT5, BMT6 or a functional portion thereof having methyl transferase activity. In some embodiments, the methyl transferase fusion partner is selected from DNMT3a, DNMT3b, DNMT1, and a functional portion thereof having methyl transferase activity. In some embodiments, the methyl transferase fusion partner is an RNA methyl transferase fusion partner. In some embodiments, the RNA methyl transferase fusion partner is selected from DNMT2, NSUN, and a functional portion thereof having methyl transferase activity.

In some embodiments, a methyl transferase fusion partner comprises a Class I methyl transferase, or a class II methyl transferase. In some embodiments, a methyl transferase fusion partner can comprise a histone methyl transferase, an N-terminal methyl transferase, a DNA methyl transferase, an RNA methyl transferase, a natural produce methyl transferase, a non-S-adenosyl methionine (SAM) dependent methyl transferase, or a radical SAM methyl transferase.

In some embodiments, a methyl transferase fusion partner can comprise a DNA methylases such as HhaI DNA m5c-methyltransferase (M.HhaI), DNMT1, DNMT3a, DNMT3b, METI, DRM3 (plants), ZMET2, CMT1, CMT2 (plants). In some embodiments, a methyl transferases fusion partner can comprise Pr-SET7/8, SUV4-20H1, RIZ1, and the like.

TABLE 2 shows exemplary methyl transferase fusion partners and their amino acid sequences. In some embodiments, a fusion partner comprises an amino acid sequence that is at least 75%, at least 80%, at least 85%, at least 90%, at least 95%, at least 98%, or at least 99% identical to any one of the methyl transferase fusion partner sequences recited in TABLE 2.

In some embodiments, a DNA alkylating fusion partner is an N-alkylating fusion partner. In some embodiments, the N-alkylating fusion partner, upon contact with a DNA molecule, performs an N-alkylation of a guanine present in the DNA molecule at position N7 of the guanine, thereby producing a 7-methylguanine in the DNA molecule. In some embodiments, the N-alkylating fusion partner comprises METTL1, WDR4, or combination thereof.

### Repair Inhibitor Fusion Partners

Disclosed herein is a repair inhibitor fusion partner. In some embodiments, a fusion effector protein comprises: (a) a repair inhibitor fusion partner; and (b) an effector protein as described herein, wherein the repair inhibitor fusion partner is linked to the effector protein via a linker.

In some embodiments, the repair inhibitor fusion partner is also referred as a repair inhibitor. In some embodiments, the repair inhibitor fusion partner inhibits O-Linked N-Acetylglucosamine (GlcNAc) Transferase (also known as O-GlcNAc transferase), encoded by the OGT gene. In some embodiments, the repair inhibitor fusion partner inhibits O⁶-alkylguanine DNA alkyltransferase (also known as AGT and ADA), a protein encoded by the MGMT gene. In some embodiments, the repair inhibitor fusion partner inhibits nucleotide excision repair.

### Deaminase Fusion Partners

Disclosed herein is a deaminase fusion partner. In some embodiments, the deaminase fusion partner is also referred as an engineered deaminase enzyme. In some embodiments, the deaminase fusion partner, upon contact with a double stranded DNA molecule, deaminates methyl cytosine residues of the double stranded DNA molecule. In some embodiments, the deaminase fusion partner deaminates methyl cytosine residues of the double stranded DNA molecule at a greater rate than cytosine residues of the double stranded DNA molecule. In some embodiments, the deaminase fusion partner is an activation-induced deaminase (AID) fusion partner. In some embodiments, the AID fusion partner comprises an APOBEC3A deaminase domain. In some embodiments, the deaminase fusion partner deaminates methyl cytosine residues of the double stranded DNA molecule about 2-fold to about 10-fold faster than cytosine residues of the double stranded DNA molecule. In some embodiments, the deaminase fusion partner deaminates methyl cytosine residues of the double stranded DNA molecule about 4-fold faster than cytosine residues of the double stranded DNA molecule.

TABLE 2 shows exemplary deaminase fusion partners and their amino acid sequences. In some embodiments, the fusion partner comprises an amino acid sequence that is at least 75%, at least 80%, at least 85%, at least 90%, at least 95%, at least 98%, or at least 99% identical to any one of the deaminase fusion partner sequences recited in TABLE 2.

In some embodiments, a deaminase fusion partner is a cytosine deaminating fusion partner. In some embodiments, the cytosine deaminating fusion partner, upon contact with a DNA molecule, performs a deamination of a cytosine present in the DNA molecule, thereby producing a deoxyuridine in the DNA molecule.

In some embodiments, a fusion effector protein is a cytosine modifying fusion protein, comprising: (a) a deaminase fusion partner as described herein; and (b) an effector protein as described herein. In some embodiments, the cytosine modifying fusion protein further comprises a methyl transferase fusion partner as described herein. In some embodiments, the cytosine modifying fusion protein comprises a linker that links the methyl transferase to the deaminase enzyme. In some embodiments, the cytosine modifying fusion protein further comprises a thymine DNA glycosylase inhibitor fusion partner. In some embodiments, at least one of the deaminase fusion partner, the methyl transferase enzyme fusion partner, and the thymine DNA glycosylase inhibitor fusion partner is linked to the effector protein via a linker.

### Thymine DNA Glycosylase Inhibitor Fusion Partners

Disclosed herein is a thymine DNA glycosylase inhibitor fusion partner. In some embodiments, the thymine DNA glycosylase inhibitor fusion partner is also referred as a thymine DNA glycosylase inhibitor. In some embodiments, the thymine DNA glycosylase inhibitor fusion partner can inhibit native thymine DNA glycosylases of a subject.

In some embodiments, a fusion effector protein comprises: (a) a thymine DNA glycosylase inhibitor fusion partner; and (b) an effector protein as described herein, wherein the thymine DNA glycosylase inhibitor fusion partner is linked to the effector protein via a linker.

### Terminal Deoxynucleotidyl Transferase (TdT) Fusion Partners

Disclosed herein is a terminal deoxynucleotidyl transferase (TdT) fusion partner. In some embodiments, the TdT fusion partner, upon contact with a DNA molecule, a DNA molecule comprising an overhang is generated.

In some embodiments, a fusion effector protein comprises: (a) a TdT fusion partner; and (b) an effector protein as described herein, wherein the TdT fusion partner is linked to the effector protein via a linker. In such embodiments, the effector protein performs a double stranded break (DSB). In such embodiments, the DSB is created by two single stranded breaks.

### RNA Pseudouridylation Fusion Partners

Disclosed herein is an RNA pseudouridylation fusion partner. In some embodiments, the pseudouridylation fusion partner, upon contact with a mRNA transcript, effects pseudouridylation of a uridine present in a target sequence of an mRNA transcript. Alternatively, in some embodiments, the pseudouridylation fusion partner converts the uridine to pseudouridine. In some embodiments, the conversion of the uridine to the pseudouridine converts the nonsense codon to a sense codon. In some embodiments, the sense codon is serine, threonine, tyrosine, or phenylalanine.

In some embodiments, a fusion effector protein, comprising: (a) an effector protein; (b) an RNA pseudouridylation fusion partner.

### Oxidizing Fusion Partners

Disclosed herein is an oxidizing fusion partner. In some embodiments, the oxidizing fusion partner, upon contact with a DNA molecule, performs an oxidation of a guanine present in the DNA molecule, thereby producing an 8-oxoguanine in the DNA molecule. In such embodiments, the oxidizing fusion partner comprises xanthine oxidase.

In some embodiments, a fusion effector protein is a fusion protein, comprising: (a) an effector protein; (b) an oxidizing fusion partner.

### Apurinic or Apyrimidinic Site Generating Fusion Partners

Disclosed herein is an apurinic or apyrimidinic site generating fusion partner. In some embodiments, the apurinic or apyrimidinic site generating fusion partner, upon contact with a DNA molecule, generates an apurinic or apyrimidinic site in the DNA molecule. In such embodiments, the apurinic or apyrimidinic site generating fusion partner comprises a DNA glycosylase.

In some embodiments, a fusion effector protein is a fusion protein, comprising: (a) an effector protein; (b) an apurinic or apyrimidinic site generating fusion partner.

### Ribonucleotide Reductase Fusion Partners

Disclosed herein is a ribonucleotide reductase fusion partner. In some embodiments, the ribonucleotide reductase fusion partner, upon contact with a DNA molecule, converts a ribonucleotide triphosphate (NTP) into a deoxyribonucleotide triphosphate (dNTP). In such embodiments, the ribonucleotide reductase fusion partner comprises a ribonucleotide reductase.

In some embodiments, a fusion effector protein is a fusion protein, comprising: (a) an effector protein; (b) a ribonucleotide reductase fusion partner.

### Linkers

In general, effector proteins and fusion partners of a fusion effector protein are connected via a linker. The linker may comprise or consist of a covalent bond. The linker may comprise or consist of a chemical group. In some embodiments, the linker comprises an amino acid. In general, the linker connects a terminus of the effector protein to a terminus of the fusion partner. In some embodiments, the carboxy terminus of the effector protein is linked to the amino terminus of the fusion partner. In some embodiments, the carboxy terminus of the fusion partner is linked to the amino terminus of the effector protein.

In some embodiments, fusion effector proteins disclosed herein comprise a linker, wherein the linker comprises or consists of a peptide. The peptide may comprise a region of rigidity (e.g., beta sheet, alpha helix), a region of flexibility, or any combination thereof. In some embodiments, the linker comprises small amino acids, such as glycine and alanine, that impart linker flexibility. In some embodiments, the linker comprises amino acids that impart linker rigidity, such as valine and isoleucine. These linkers may be produced by using synthetic, linker-encoding oligonucleotides to couple the proteins, or may be encoded by a nucleic acid sequence encoding a fusion effector protein (e.g., an effector protein coupled to a fusion partner). Linkers may comprise glycine(s), serine(s), and combinations thereof. Examples of linker proteins include glycine polymers (G)n (SEQ ID NO: 500), glycine-serine polymers (including, for example, (GS)n (SEQ ID NO: 501), GSGGSn (SEQ ID NO: 502), GGSGGSn (SEQ ID NO: 503), and GGGSn (SEQ ID NO: 504), where n is an integer of at least one), glycine-alanine polymers, and alanine-serine polymers. Exemplary linkers may comprise amino acid sequences including, but not limited to, GGSG (SEQ ID NO: 505), GGSGG (SEQ ID NO: 506), GSGSG (SEQ ID NO: 507), GSGGG (SEQ ID NO: 508), GGGSG (SEQ ID NO: 509), GGGSGGS (SEQ ID NO: 510), and GSSSG (SEQ ID NO: 511).

In some embodiments, the linker comprises or consists of at least a portion of the sequence: GSPAGSPTSTEEGTSESATPESGPGTSTEPSEGSAPGSPAGSPTSTEEGTSTEPSEGSAP GTSTEPSEGSAPGTSESATPESGPGSEPATSGSETPGSEPATSGSETPGSPAGSPTSTEE GTSESATPESGPGTSTEPSEGSAPGTSTEPSEGSAPGSPAGSPTSTEEGTSTEPSEGSAP GTSTEPSEGSAPGTSESATP (SEQ ID NO: 512). The linker may comprise or consist of at least 10, at least 15, at least 20, at least 25, at least 30, at least 35, at least 40, at least 50, at least 55, at least 60, at least 65, at least 70, at least 75, at least 80, at least 85, at least 90, or at least 100 contiguous amino acids of SEQ ID NO: 512. The linker may comprise or consist of at least 100, at least 110, at least 120, at least 130, at least 140, at least 150, at least 160, at least 170, at least 180, or at least 190 contiguous amino acids of SEQ ID NO: 512. The linker may comprise a sequence that is at least 30%, at least 40%, at least 50%, at least 60%, at least 70%, at least 80% or at least 90% to at least an equal length portion of SEQ ID NO: 512.

In some embodiments, the amino acid sequence of the linker is GSPAGSPTST (SEQ ID NO: 513). In some embodiments, the amino acid sequence of the linker is 70%, 80%, or 90% identical to GSPAGSPTST (SEQ ID NO: 513). In some embodiments, the amino acid sequence of the linker is GSPAGSPTSTEEGTSESATP (SEQ ID NO: 514). In some embodiments, the amino acid sequence of the linker is 70%, 75%, 80%, 85%, 90%, or 95% identical to GSPAGSPTSTEEGTSESATP (SEQ ID NO: 514). In some embodiments, the linker is an XTEN40 linker (e.g., GSPAGSPTSTEEGTSESATPESGPGTSTEPSEGSAPGSPA SEQ ID NO: 515). In some embodiments, the amino acid sequence of the linker is GSPAGSPTSTEEGTSESATPESGPGTSTEPSEGSAPGSPA (SEQ ID NO: 515). In some embodiments, the amino acid sequence of the linker is at least 70%, at least 75%, at least 80%, at least 85%, at least 90%, or at least 95% identical to GSPAGSPTSTEEGTSESATPESGPGTSTEPSEGSAPGSPA (SEQ ID NO: 515). In some embodiments, the linker is an XTEN80 linker (e.g., GSPAGSPTSTEEGTSESATPESGPGTSTEPSEGSAPGSPAGSPTSTEEGTSTEPSEGSAP GTSTEPSEGSAPGTSESATP (SEQ ID NO: 516). In some embodiments, the amino acid sequence of the linker is GSPAGSPTSTEEGTSESATPESGPGTSTEPSEGSAPGSPAGSPTSTEEGTSTEPSEGSAP GTSTEPSEGSAPGTSESATP (SEQ ID NO: 516). In some embodiments, the amino acid sequence of the linker is at least 70%, at least 75%, at least 80%, at least 85%, at least 90%, or at least 95% identical to GSPAGSPTSTEEGTSESATPESGPGTSTEPSEGSAPGSPAGSPTSTEEGTSTEPSEGSAP GTSTEPSEGSAPGTSESATP (SEQ ID NO: 516).

In some embodiments, the amino acid sequence of the linker is GSGSPAGSPTSTRSGGGSGTS (SEQ ID NO: 517). In some embodiments, the amino acid sequence of the linker is at least 70%, at least 75%, at least 80%, at least 85%, at least 90%, or at least 95% identical to GSGSPAGSPTSTRSGGGSGTS (SEQ ID NO: 517).

In some embodiments, linkers comprise or consist of 4 to 60, 6 to 55, 8 to 50, 10 to 45, 12 to 40, 14 to 35, 16 to 30, 18 to 25 linked amino acids. In some embodiments, linkers comprise or consist of 1 to 10, 10 to 20, 20 to 30, 30 to 40, 40 to 50, or 50 to 60 linked amino acids. In some embodiments, linkers comprise or consist of 5 to 10, 10 to 15, 15 to 20, 20 to 25, 25 to 30, 30 to 35, 35 to 40, 40 to 45, 45 to 50, 50 to 55 or 55 to 60 linked amino acids. In some embodiments, linkers comprise or consist of about 5, about 10, about 15, about 20, about 25, about 30, about 35, about 40, about 45, about 50, about 55 or about 60 amino acids.

In some embodiments, linkers comprise or consists of a non-peptide linker. Non-limiting examples of non-peptide linkers are linkers comprising polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacrylamide, polyacrylate, polycyanoacrylates, lipid polymers, chitins, hyaluronic acid, heparin, an alkyl linker, or a combination thereof.

In some embodiments, linkers comprise or consist of a nucleic acid. In some embodiments, the nucleic acid comprises DNA. In some embodiments, the nucleic acid comprises RNA. In some embodiments, the effector protein and the fusion partner each interact with the nucleic acid, the nucleic acid thereby linking the effector protein and the fusion partner. In some embodiments, the nucleic acid serves as a scaffold for both the effector protein and the fusion partner to interact with, thereby linking the effector protein and the fusion partner. Such nucleic acids include those described by Tadakuma et al., (2016), Progress in Molecular Biology and Translational Science, Volume 139, 2016, Pages 121-163, incorporated herein by reference.

In some embodiments, the fusion effector protein or the guide nucleic acid comprises a chemical modification that allows for direct crosslinking between the guide nucleic acid or the effector protein and the fusion partner. By way of non-limiting example, the chemical modification may comprise any one of a SNAP-tag, CLIP-tag, ACP-tag, Halo-tag, and an MCP-tag. In some embodiments, modifications are introduced with a Click Reaction, also known as Click Chemistry. The Click reaction may be copper dependent or copper independent.

In some embodiments, guide nucleic acids comprise an aptamer. The aptamer may serve as a linker between the effector protein and the fusion partner by interacting non-covalently with both. In some embodiments, the aptamer binds a fusion partner, wherein the fusion partner is a transcriptional activator. In some embodiments, the aptamer binds a fusion partner, wherein the fusion partner is a transcriptional inhibitor. In some embodiments, the aptamer binds a fusion partner, wherein the fusion partner comprises a base editor. In some embodiments, the aptamer binds the fusion partner directly. In some embodiments, the aptamer binds the fusion partner indirectly. Aptamers may bind the fusion partner indirectly through an aptamer binding protein. By way of non-limiting example, the aptamer binding protein may be MS2 and the aptamer sequence may be ACATGAGGATCACCCATGT (SEQ ID NO: 810); the aptamer binding protein may be PP7 and the aptamer sequence may be GGAGCAGACGATATGGCGTCGCTCC (SEQ ID NO: 811); or the aptamer binding protein may be BoxB and the aptamer sequence may be GCCCTGAAGAAGGGC (SEQ ID NO: 812).

In some embodiments, fusion effector proteins do not comprise a linker. In some embodiments, the fusion partner is located within effector protein. For example, the fusion partner may be a domain of a fusion partner protein that is internally integrated into the effector protein. In other words, the fusion partner may be located between the 5′ and 3′ ends of the effector protein without disrupting the ability of an RNP comprising the fusion effector protein to recognize/bind a target nucleic acid. In some embodiments, the fusion partner replaces a portion of the effector protein. In some embodiments, the fusion partner replaces a domain of the effector protein. In some embodiments, the fusion partner does not replace a portion of the effector protein.

### Additional/Optional Fusion Effector Features

In some embodiments, a fusion effector protein comprises a subcellular localization signal. In some embodiments, a fusion partner comprises a subcellular localization signal. In some embodiments, an effector protein comprises a subcellular localization signal. In some embodiments, a subcellular localization signal can be a nuclear localization signal (NLS). In some embodiments, the NLS facilitates localization of a nucleic acid, protein, or small molecule to the nucleus, when present in a cell that contains a nuclear compartment. TABLE 3 lists exemplary NLS sequences. In some embodiments, the subcellular localization signal is a nuclear export signal (NES), a sequence to keep an effector protein retained in the cytoplasm, a mitochondrial localization signal for targeting to the mitochondria, a chloroplast localization signal for targeting to a chloroplast, an ER retention signal, and the like. In some embodiments, a fusion protein does not comprise a nuclear localization signal so that the effector protein is not targeted to the nucleus, which can be advantageous depending on the circumstance (e.g., when the target nucleic acid is an RNA that is present in the cytosol).

In some embodiments, the heterologous polypeptide is an endosomal escape peptide (EEP). An EEP is an agent that quickly disrupts the endosome in order to minimize the amount of time that a delivered molecule, such an effector protein, spends in the endosome-like environment, and to avoid getting trapped in the endosomal vesicles and degraded in the lysosomal compartment. An exemplary EEP is set forth in TABLE 3.

TABLE 3 provides illustrative sequences of exemplary fusion partners that are useful in the compositions, systems and methods described herein.

TABLE 3
EXEMPLARY HETEROLOGOUS POLYPEPTIDE SEQUENCES

| SEQ ID | | |
|---|---|---|
| NO: | Description | Sequence |
| 227 | NLS | KR(K/R)R |
| 228 | NLS | (P/R)XXKR(^{∧}D/E)(K/R) |
| 229 | NLS | KRX(W/F/Y)XXAF |
| 230 | NLS | (R/P)XXKR(K/R)(^{∧}D/E) |
| 231 | NLS | LGKR(K/R)(W/F/Y) |
| 232 | NLS | KRX₁₀K(K/R)(K/R) |
| 233 | NLS | K(K/R)RK |
| 234 | NLS | KRX₁₁K(K/R)(K/R) |
| 235 | NLS | KRX₁₂K(K/R)(K/R) |
| 236 | NLS | KRX₁₀K(K/R)X(K/R) |
| 237 | NLS | KRX₁₁K(K/R)X(K/R) |
| 238 | NLS | KRX₁₂K(K/R)X(K/R) |
| 239 | NLS | APKKKRKVGIHGVPAA |
| 600 | NLS | KRPAATKKAGQAKKKK |
| 601 | NLS | PKKKRKV |
| 602 | NLS | LPPLERLTL |
| 603 | NLS | MPKKKRKVGIHGVPAA |
| 240 | EEP | GLFXALLXLLXSLWXLLLXA |

*wherein X is any naturally occurring amino acid; and ^{∧}D/E is any naturally occurring amino acid except Asp or Glu

A fusion effector protein disclosed herein, or a variant thereof may comprise a nuclear localization signal (NLS). In some embodiments, the NLS may comprises a sequence of KRPAATKKAGQAKKKK (SEQ ID NO: 600). In some embodiments, the NLS comprises a sequence of PKKKRKV (SEQ ID NO: 601). In some embodiments, the NLS comprises a sequence of LPPLERLTL (SEQ ID NO: 602). In some embodiments, the NLS comprises a sequence of MPKKKRKVGIHGVPAA (SEQ ID NO: 603). A fusion effector protein may be codon optimized for expression in a specific cell, for example, a bacterial cell, a plant cell, a eukaryotic cell, an animal cell, a mammalian cell, or a human cell. In some embodiments, the fusion effector protein is codon optimized for a human cell. The NLS may be located at a variety of locations, including, but not limited to 5′ of the effector protein, 5′ of the fusion partner, 3′ of the effector protein, 3′ of the fusion partner, between the effector protein and the fusion partner, within the fusion partner, within the effector protein.

In some embodiments the fusion partner is fused to an RNA-binding domain. In some embodiments the RNA-binding domain is a coat protein. In some embodiments, the coat protein is at least one of: MS2, PP7 or Qbeta as described in WO2019178428A1, which is hereby incorporated by reference in its entirety. In some embodiments, fusion proteins comprise an RNA-binding domain fused to the fusion partner. Examples of RNA-binding domains include but are not limited to MS2, PP7, or Qbeta as described in WO2019178428, which is hereby incorporated by reference in its entirety.

In some embodiments, a heterologous peptide or heterologous polypeptide comprises a chloroplast transit peptide (CTP), also referred to as a chloroplast localization signal or a plastid transit peptide, which targets the effector protein to a chloroplast. Chromosomal transgenes from bacterial sources may require a sequence encoding a CTP sequence fused to a sequence encoding an expressed protein (e.g., the effector protein) if the expressed protein is to be compartmentalized in the plant plastid (e.g., chloroplast). The CTP may be removed in a processing step during translocation into the plastid. Accordingly, localization of an effector protein to a chloroplast is often accomplished by means of operably linking a polynucleotide sequence encoding a CTP sequence to the 5′ region of a polynucleotide encoding the exogenous protein.

In some embodiments, the fusion protein comprises a protein transduction domain (PTD), which may also be referred to as a cell penetrating peptide (CPP). In some embodiments, the PTD is a polynucleotide, carbohydrate, or organic or inorganic compound that facilitates transversing a lipid bilayer, micelle, cell membrane, organelle membrane, or vesicle membrane. In some embodiments, the PTD is attached to another molecule. In some embodiments, the other molecule is a small polar molecule. In some embodiments, the other molecule facilitates traversing a membrane. In some embodiments, the other molecule is a large macromolecule. In some embodiments, the other molecule is a nanoparticle. In some embodiments, the traversing of a membrane refers to going from extracellular space to intracellular space or cytosol within an organelle.

Further suitable fusion partners include, but are not limited to, proteins (or fragments/domains thereof) that are boundary elements (e.g., CTCF), proteins and fragments thereof that provide periphery recruitment (e.g., Lamin A, Lamin B, etc.), and protein docking elements (e.g., FKBP/FRB, Pil1/Aby1, etc.).

In some embodiments, a fusion protein or fusion partner comprises a protein tag. In some embodiments, the protein tag is referred to as purification tag or a fluorescent protein. The protein tag may be detectable for use in detection of the effector protein and/or purification of the effector protein. Accordingly, in some embodiments, compositions, systems and methods comprise a protein tag or use thereof. Any suitable protein tag may be used depending on the purpose of its use. Non-limiting examples of protein tags include a fluorescent protein, a histidine tag, e.g., a 6XHis tag; a hemagglutinin (HA) tag; a FLAG tag; a Myc tag; and maltose binding protein (MBP). In some embodiments, the protein tag is a portion of MBP that can be detected and/or purified. Non-limiting examples of fluorescent proteins include green fluorescent protein (GFP), yellow fluorescent protein (YFP), red fluorescent protein (RFP), cyan fluorescent protein (CFP), mCherry, and tdTomato.

In some embodiments, the fusion protein comprises an apurinic/apyrmidinic (AP)-binding domain. The AP-binding domain can comprise any domain with covalent binding activity at an AP site, for example, an SOS response-associated peptidase (SRAP) domain, such as the SRAP domain of 5-hydroxymethylcytosine (5hmC) binding, ESC-specific (HMCES) as described in WO2020209959 and Mohni et al. 2019, Cell 176, 144-153, which are hereby incorporated by reference in its entirety. In some embodiments, the AP-binding domain comprises an SOS response-associated peptidase (SRAP) domain. In some embodiments, the SRAP domain is from 5-hydroxymethyl cytosine binding, ESC specific (HMCES) or YedK, or a variant thereof. In some embodiments, the AP-binding domain comprises an SRAP domain sequence as described in WO2020209959.

In some embodiments, a fusion protein described herein comprises a propeptide linked to a bioluminescent protein as described in U.S. Pat. Nos. 10,370,697 and 9,657,329, which are hereby incorporated by reference in its entirety.

In some embodiments, the fusion protein comprises one or more additional features that can be helpful for a variety of diagnostic, detection, and gene editing functionalities. Non-limiting examples of additional features include, but are not limited to, inhibitors, cytoplasmic localization sequences, export sequences (e.g., nuclear export sequences), or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Exemplary additional useful features of fusion proteins described herein can be found in WO2021050571, which is hereby incorporated by reference in its entirety. In some embodiments, the fusion protein comprises additional functional domains. Non-limiting examples of functional domains include, but are not limited to, Kriippel associated box (KRAB), VP64, VP16, Fok1, P65, HSF1, MyoD1, and biotin-APEX as described in WO2021007563, which is hereby incorporated by reference in its entirety.

A fusion protein may comprise one or more additional amino acid sequences comprising heterologous domain(s), and optionally a linker sequence between any two domains, such as between the effector protein and a first heterologous domain. Examples of protein domains that may be fused to a effector protein herein include, without limitation, epitope tags (e.g., histidine (His), V5, FLAG, influenza hemagglutinin (HA), myc, VSV-G, thioredoxin (Trx)), and reporters (e.g., glutathione-5-transferase (GST), horseradish peroxidase (HRP), chloramphenicol acetyltransferase (CAT), beta-galactosidase, beta-glucuronidase (GUS), luciferase, green fluorescent protein (GFP), HcRed, DsRed, cyan fluorescent protein (CFP), yellow fluorescent protein (YFP), and blue fluorescent protein (BFP)) as described in WO2020123887, which is hereby incorporated in its entirety by reference.

In some embodiments, a fusion protein may comprise one or more additional amino acid sequences with one or more domains having one or more of the following activities: methylase activity, demethylase activity, transcription activation activity (e.g., VP 16 or VP64), transcription repression activity, transcription release factor activity, histone modification activity, RNA cleavage activity and nucleic acid binding activity as described in WO2020123887, which is hereby incorporated in its entirety by reference.

In some embodiments, the fusion protein comprises a CRISPR subtype E protein (Cse). Cse proteins can be essential for protection against lambda phage challenge. Multiple Cse proteins can form a cascade as described in U.S. Pat. No. 10,711,257, which is hereby incorporated by reference in its entirety.

### II. Guide Nucleic Acids

The compositions, systems, and methods of the present disclosure may comprise a guide nucleic acid or a use thereof. In general, a guide nucleic acid is a nucleic acid molecule that binds to an effector protein, thereby forming a ribonucleoprotein complex (RNP). Unless otherwise indicated, compositions, systems and methods comprising guide nucleic acids or uses thereof, as described herein and throughout, include DNA molecules, such as expression vectors, that encode a guide nucleic acid. Accordingly, compositions, systems, and methods of the present disclosure comprise a guide nucleic acid or a nucleotide sequence encoding the guide nucleic acid.

The compositions, systems, and methods of the present disclosure may comprise a guide nucleic acid, a nucleic acid encoding the guide nucleic acid, or a use thereof. Unless otherwise indicated, compositions, systems and methods comprising guide nucleic acids or uses thereof, as described herein and throughout, include DNA molecules, such as expression vectors, that encode a guide nucleic acid. Guide nucleic acids are also referred to herein as “guide RNA.” A guide nucleic acid, as well as any components thereof (e.g., spacer sequence, repeat sequence, linker nucleotide sequence, handle sequence, intermediary sequence etc.) may comprise one or more deoxyribonucleotides, ribonucleotides, biochemically or chemically modified nucleotides (e.g., one or more engineered modifications as described herein), or any combinations thereof. Such nucleotide sequences described herein may be described as a nucleotide sequence of either DNA or RNA, however, no matter the form the sequence is described, it is readily understood that such nucleotide sequences can be revised to be RNA or DNA, as needed, for describing a sequence within a guide nucleic acid itself or the sequence that encodes a guide nucleic acid, such as a nucleotide sequence described herein for a vector. Similarly, disclosure of the nucleotide sequences described herein also discloses the complementary nucleotide sequence, the reverse nucleotide sequence, and the reverse complement nucleotide sequence, any one of which can be a nucleotide sequence for use in a guide nucleic acid as described herein.

A guide nucleic acid may comprise a naturally occurring sequence. A guide nucleic acid may comprise a non-naturally occurring sequence, wherein the sequence of the guide nucleic acid, or any portion thereof, may be different from the sequence of a naturally occurring guide nucleic acid. A guide nucleic acid of the present disclosure comprises one or more of the following: a) a single nucleic acid molecule; b) a DNA base; c) an RNA base; d) a modified base; e) a modified sugar; f) a modified backbone; and the like. A guide nucleic acid may be chemically synthesized or recombinantly produced by any suitable methods. Guide nucleic acids and portions thereof may be found in or identified from a CRISPR array present in the genome of a host organism or cell.

The guide nucleic acid may comprise a first region complementary to a target nucleic acid (FR1) and a second region that is not complementary to the target nucleic acid (FR2). In some embodiments, FR1 is located 5′ to FR2 (FR1-FR2). In some embodiments, FR2 is located 5′ to FR1 (FR2-FR1). In some embodiments, FR1 comprises a spacer sequence, wherein the spacer sequence can interact in a sequence-specific manner with (e.g., has complementarity with, or can hybridize to a target sequence in) a target nucleic acid. In some embodiments, FR2 comprises one or more repeat sequences or intermediary sequence. In some embodiments, an effector protein binds to at least a portion of the FR2.

In some embodiments, the guide nucleic acid comprises 10, 11, 12, 13, 14, 15, 16, 17, 18, 19, 20, 21, 22, 23, 24, 25, 26, 27, 28, 29, or 30 linked nucleosides. In general, a guide nucleic acid comprises at least linked nucleosides. In some embodiments, a guide nucleic acid comprises at least 25 linked nucleosides. A guide nucleic acid may comprise 10 to 50 linked nucleosides. In some embodiments, the guide nucleic acid comprises or consists essentially of about 12 to about 80 linked nucleosides, about 12 to about 50, about 12 to about 45, about 12 to about 40, about 12 to about 35, about 12 to about 30, about 12 to about 25, from about 12 to about 20, about 12 to about 19, about 18 to about 20, about 19 to about 20, about 19 to about 22, about 19 to about 25, about 19 to about 30, about 19 to about 35, about 19 to about 40, about 19 to about 45, about 19 to about 50, about 19 to about 60, about 20 to about 25, about 20 to about 30, about 20 to about 35, about 20 to about 40, about 20 to about 45, about 20 to about 50, or about 20 to about 60 linked nucleosides. In some embodiments, the guide nucleic acid has about 10 to about 60, about 20 to about 50, or about 30 to about 40 linked nucleosides.

A guide RNA generally comprises a CRISPR RNA (crRNA), at least a portion of which is complementary to a target sequence of a target nucleic acid. In some embodiments, the guide RNA comprises a trans-activating CRISPR RNA (tracrRNA) that interacts with the effector protein. In some embodiments, the composition does not comprise a tracrRNA. In some embodiments, the guide RNA is a single guide RNA (sgRNA) (e.g., a crRNA linked to a tracrRNA). In some embodiments, a crRNA and tracrRNA function as two separate, unlinked molecules. The term “guide RNA,” as well as crRNA and tracrRNA, includes guide nucleic acids comprising DNA bases and RNA bases. The guide RNA may be chemically synthesized or recombinantly produced. The sequence of the guide nucleic acid, or a portion thereof, may be different from the sequence of a naturally occurring nucleic acid.

In some embodiments, fusion effector proteins are targeted by a guide nucleic acid (e.g., a guide RNA) to a specific location in the target nucleic acid where they exert locus-specific regulation. Non-limiting examples of locus-specific regulation include blocking RNA polymerase binding to a promoter (which selectively inhibits transcription activator function), and/or modifying local chromatin (e.g., when a fusion sequence is used that modifies the target nucleic acid or modifies a protein associated with the target nucleic acid). The guide RNA may bind (e.g., hybridize) to a target nucleic acid (e.g., a single strand of a target nucleic acid) or a portion thereof, an amplicon thereof, or a portion thereof. By way of non-limiting example, a guide nucleic acid may bind (e.g., hybridize) to a target nucleic acid, such as DNA or RNA, from a cancer gene or gene associated with a genetic disorder, or an amplicon thereof, as described herein.

The guide nucleic acid (e.g., a non-naturally occurring guide nucleic acid) can be selected from a group of guide nucleic acids that have been tiled against the nucleic acid sequence of a strain of an infection or genomic locus of interest. The guide nucleic acid can be selected from a group of guide nucleic acids that have been tiled against the nucleic acid sequence of a strain of HPV 16 or HPV1 8. Often, guide nucleic acids that are tiled against the nucleic acid of a strain of an infection or genomic locus of interest can be pooled for use in a method described herein. Often, these guide nucleic acids are pooled for detecting a target nucleic acid in a single assay. The pooling of guide nucleic acids that are tiled against a single target nucleic acid can enhance the detection of the target nucleic using the methods described herein. The pooling of guide nucleic acids that are tiled against a single target nucleic acid can ensure broad coverage of the target nucleic acid within a single reaction using the methods described herein. The tiling, for example, is sequential along the target nucleic acid. Sometimes, the tiling is overlapping along the target nucleic acid. In some instances, the tiling comprises gaps between the tiled guide nucleic acids along the target nucleic acid. In some instances, the tiling of the guide nucleic acids is non-sequential. Often, a method for detecting a target nucleic acid comprises contacting a target nucleic acid to a pool of guide nucleic acids and a programmable nuclease, wherein a guide nucleic acid sequence of the pool of guide nucleic acids has a sequence selected from a group of tiled guide nucleic acid that correspond to nucleic acid sequence of a target nucleic acid; and assaying for a signal produce by cleavage of at least some nucleic acids of a reporter of a population of nucleic acids of a reporter. Pooling of guide nucleic acids can ensure broad spectrum identification, or broad coverage, of a target species within a single reaction. This can be particularly helpful in diseases or indications, like sepsis, that may be caused by multiple organisms as described in WO 2020142754, which is hereby incorporated by reference in its entirety.

The target gene may be associated with a disease. In some embodiments, the guide nucleic acid directs that base editor to or near a mutation in the sequence of a target gene. The mutation may be the deletion of one more nucleotides. The mutation may be the addition of one or more nucleotides. The mutation may be the substitution of one or more nucleotides. The mutation may be the insertion, deletion or substitution of a single nucleotide, also referred to as a point mutation. The point mutation may be a SNP. The mutation may be associated with a disease. In some aspects, the single nucleotide polymorphism (SNP) comprises a HERC2 SNP. In some aspects, the single nucleotide polymorphism (SNP) is associated with an increased risk or decreased risk of cancer. In some aspects, the target nucleic acid comprises a single nucleotide polymorphism (SNP), and wherein the detectable signal is higher in the presence of a guide nucleic acid that is 100% complementary to the target nucleic acid comprising the single nucleotide polymorphism (SNP) than in the presence of a guide nucleic acid that is less than 100% complementary to the target nucleic acid comprising the single nucleotide polymorphism (SNP). See WO2020142754, which is hereby incorporated by reference in its entirety. In some embodiments, the guide nucleic acid directs the fusion partner to bind a target sequence within the target nucleic acid that is within 0, 1, 2, 3, 4, 5, 6, 7, 8, 9, 10, 11, 12, 13, 14, 15, 16, 17, 18, 19, or 20 nucleotides of the mutation. In some embodiments, the guide nucleic acid comprises a sequence that is identical, complementary or reverse complementary to a target sequence of a target nucleic acid that comprises the mutation. In some embodiments, the guide nucleic acid comprises a sequence that is identical, complementary or reverse complementary to a target sequence of a target nucleic acid that is within 1, 2, 3, 4, 5, 6, 7, 8, 9, 10, 11, 12, 13, 14, 15, 16, 17, 18, 19, or 20 nucleotides of the mutation.

In some embodiments, an effector protein cleaves a precursor RNA (“pre-crRNA”) to produce a guide RNA, also referred to as a “mature guide RNA.” An effector protein that cleaves pre-crRNA to produce a mature guide RNA is said to have pre-crRNA processing activity. In some embodiments, a repeat region of a guide RNA comprises mutations or truncations relative to respective regions in a corresponding pre-crRNA.

In some embodiments, the guide nucleic acid comprises a nucleotide sequence as described herein (e.g., TABLE 5 and TABLE 7). Such nucleotide sequences described herein (e.g., TABLE 5 and TABLE 7) may be described as a nucleotide sequence of either DNA or RNA, however, no matter the form the sequence is described, it is readily understood that such nucleotide sequences can be revised to be RNA or DNA, as needed, for describing a sequence within a guide nucleic acid itself or the sequence that encodes a guide nucleic acid, such as a nucleotide sequence described herein for a viral vector. Similarly, disclosure of the nucleotide sequences described herein (e.g., TABLE 5 and TABLE 7) also discloses the complementary nucleotide sequence, the reverse nucleotide sequence, and the reverse complement nucleotide sequence, any one of which can be a nucleotide sequence for use in a guide nucleic acid as described herein.

### Repeat Region

Guide nucleic acids described herein may comprise one or more repeat regions. In some embodiments, a repeat region comprises a nucleotide sequence that is not complementary to a target sequence of a target nucleic acid. In some embodiments, a repeat region comprises a nucleotide sequence that may interact with an effector protein (e.g., repeat sequence). In some embodiments, a repeat sequence is connected to another sequence of a guide nucleic acid, such as an intermediary sequence, that is capable of non-covalently interacting with an effector protein. In some embodiments, a repeat sequence includes a nucleotide sequence that is capable of forming a guide nucleic acid-effector protein complex (e.g., a RNP complex).

In some embodiments, the repeat sequence is between 10 and 50, 12 and 48, 14 and 46, 16 and 44, and 18 and 42 nucleotides in length.

In some embodiments, a repeat sequence is adjacent to a spacer sequence. In some embodiments, a repeat sequence is followed by a spacer sequence in the 5′ to 3′ direction. In some embodiments, a repeat sequence is preceded by a spacer sequence in the 5′ to 3′ direction. In some embodiments, a repeat sequence is adjacent to an intermediary sequence. In some embodiments, a repeat sequence is 3′ to an intermediary sequence. In some embodiments, an intermediary sequence is followed by a repeat sequence, which is followed by a spacer sequence in the 5′ to 3′ direction. In some embodiments, a repeat sequence is linked to a spacer sequence and/or an intermediary sequence. In some embodiments, a guide nucleic acid comprises a repeat sequence linked to a spacer sequence and/or to an intermediary sequence, which may be a direct link or by any suitable linker, examples of which are described herein.

In some embodiments, guide nucleic acids comprise more than one repeat sequence (e.g., two or more, three or more, or four or more repeat sequences). In some embodiments, a guide nucleic acid comprises more than one repeat sequence separated by another sequence of the guide nucleic acid. For example, in some embodiments, a guide nucleic acid comprises two repeat sequences, wherein the first repeat sequence is followed by a spacer sequence, and the spacer sequence is followed by a second repeat sequence in the 5′ to 3′ direction. In some embodiments, the more than one repeat sequences are identical. In some embodiments, the more than one repeat sequences are not identical.

In some embodiments, the repeat sequence comprises two sequences that are complementary to each other and hybridize to form a double stranded RNA duplex (dsRNA duplex). In some embodiments, the two sequences are not directly linked and hybridize to form a stem loop structure. In some embodiments, the dsRNA duplex comprises 5, 10, 15, 20 or 25 base pairs (bp). In some embodiments, not all nucleotides of the dsRNA duplex are paired, and therefore the duplex forming sequence may include a bulge. In some embodiments, the repeat sequence comprises a hairpin or stem-loop structure, optionally at the 5′ portion of the repeat sequence. In some embodiments, a strand of the stem portion comprises a sequence and the other strand of the stem portion comprises a sequence that is, at least partially, complementary. In some embodiments, such sequences may have 65% to 100% complementarity (e.g., 65%, 70%, 75%, 80%, 85%, 90%, 95%, or 100% complementarity). In some embodiments, a guide nucleic acid comprises nucleotide sequence that when involved in hybridization events may hybridize over one or more segments such that intervening or adjacent segments are not involved in the hybridization event (e.g., a bulge, a loop structure or hairpin structure, etc.).

In some embodiments, a repeat sequence comprises a nucleotide sequence that is at least 65%, at least 70%, at least 75%, at least 80%, at least 85%, at least 90%, at least 95%, at least 97%, or at least 98%, at least 99%, or 100% identical to an equal length portion of any one of the repeat sequences in TABLE 7. In some embodiments, a repeat sequence comprises at least 5, at least 6, at least 7, at least 8, at least 9, at least 10, at least 11, at least 12, at least 13, at least 14, at least 15, at least 16, at least 17, at least 18, at least 19, at least 20, or at least 21 contiguous nucleotides of any one of the sequences recited in TABLE 7.

In some embodiments, a repeat sequence comprises one or more nucleotide alterations at one or more positions in the sequence recited in TABLE 7. Alternative nucleotides can be any one or more of A, C, G, T or U, or a deletion, or an insertion.

### Spacer Region

Guide nucleic acids described herein may comprise one or more spacer regions. In some embodiments, a spacer region is capable of hybridizing to a target sequence of a target nucleic acid. In some embodiments, a spacer sequence comprises a nucleotide sequence that is, at least partially, hybridizable to an equal length of a sequence (e.g., a target sequence) of a target nucleic acid (e.g., a spacer sequence). Exemplary hybridization conditions are described herein. In some embodiments, the spacer sequence may function to direct an RNP complex comprising the guide nucleic acid to the target nucleic acid for detection and/or editing. The spacer sequence may function to direct a RNP to the target nucleic acid for detection and/or editing. A spacer sequence may be complementary to a target sequence that is adjacent to a PAM that is recognizable by an effector protein described herein.

In some embodiments, a spacer sequence comprises at least 5 to about 50 contiguous nucleotides that are complementary to a target sequence in a target nucleic acid. In some embodiments, a spacer sequence comprises at least 5 to about 50 linked nucleotides. In some embodiments, a spacer sequence comprises at least 5 to about 50, at least 5 to about 25, at least about 10 to at least about 25, or at least about 15 to about 25 linked nucleotides. In some embodiments, the spacer sequence comprises 15-28 linked nucleotides. In some embodiments, a spacer sequence comprises 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleotides. In some embodiments, a spacer sequence comprises 18-20 linked nucleosides in length. In some embodiments, the spacer sequence comprises 10, 11, 12, 13, 14, 15, 16, 17, 18, 19, 20, 21, 22, 23, 24, 25, 26, 27, 28, 29, 30, or more nucleotides. In some embodiments, the first sequence is 18 linked nucleosides in length.

In some embodiments, a spacer sequence is adjacent to a repeat sequence. In some embodiments, a spacer sequence follows a repeat sequence in a 5′ to 3′ direction. In some embodiments, a spacer sequence precedes a repeat sequence in a 5′ to 3′ direction. In some embodiments, the spacer sequence(s) and the repeat sequence(s) of the guide nucleic acid are present within the same molecule. In some embodiments, the spacer(s) and repeat sequence(s) are linked directly to one another. In some embodiments, a linker is present between the spacer(s) and repeat sequences. Linkers may be any suitable linker. In some embodiments, the spacer sequence(s) and the repeat sequence(s) of the guide nucleic acid are present in separate molecules, which are joined to one another by base pairing interactions.

In some embodiments, a spacer sequence comprises a nucleotide sequence that is at least 65%, at least 70%, at least 75%, at least 80%, at least 85%, at least 90%, at least 95% or 100% complementary to a target sequence of a target nucleic acid. A spacer sequence is capable of hybridizing to an equal length portion of a target nucleic acid (e.g., a target sequence). In some embodiments, a target nucleic acid, such as DNA or RNA, may be a cancer gene or gene associated with a genetic disorder, or an amplicon thereof, as described herein. In some embodiments, a spacer sequence comprises a sequence that is at least 65%, at least 70%, at least 75%, at least 80%, at least 85%, at least 90%, at least 95% or 100% complementary to a target sequence of a target nucleic acid. In some embodiments, a target nucleic acid is a nucleic acid associated with a disease or syndrome. In some embodiments, a spacer sequence comprises a sequence that is at least 65%, at least 70%, at least 75%, at least 80%, at least 85%, at least 90%, at least 95% or 100% complementary to a target sequence of a target nucleic acid associated with a disease or syndrome. In some embodiments, the spacer sequence comprises at least 10, 11, 12, 13, 14, 15, 16, 17, 18, 19, 20, 21, 22, 23, 24, 25, 26, 27, 28, 29, or 30 contiguous nucleotides that are capable of hybridizing to the target sequence. In some embodiments, the spacer sequence comprises at least 10, 11, 12, 13, 14, 15, 16, 17, 18, 19, 20, 21, 22, 23, 24, 25, 26, 27, 28, 29, or 30 contiguous nucleotides that are complementary to the target sequence.

It is understood that the spacer sequence of a spacer sequence need not be 100% complementary to that of a target sequence of a target nucleic acid to hybridize or hybridize specifically to the target sequence. For example, the spacer sequence may comprise at least one edit, such as substituted or edited nucleotide, that is not complementary to the corresponding nucleotide of the target sequence. Spacer sequences are further described throughout herein, for examples, in the Examples section.

In some embodiments, a spacer sequence comprises a nucleotide sequence that is at least 65%, at least 70%, at least 75%, at least 80%, at least 85%, at least 90%, at least 95%, at least 97%, or at least 98%, at least 99%, or 100% identical to any one of the spacer sequences in Example #. In some embodiments, the spacer sequence comprises at least 5, at least 6, at least 7, at least 8, at least 9, at least 10, at least 11, at least 12, at least 13, at least 14, at least 15, at least 16, at least 17, at least 18, at least 19, or at least 20, or at least 21 contiguous nucleotides of any one of the sequences recited in Example 6 and 7.

### Linker for Nucleic Acids

In some embodiments, a guide nucleic acid for use with compositions, systems, and methods described herein comprises one or more linkers, or a nucleic acid encoding one or more linkers. In some embodiments, the guide nucleic acid comprises at least one, at least two, at least three, at least four, at least five, at least six, at least seven, at least eight, at least nine, or at least ten linkers. In some embodiments, the guide nucleic acid comprises one, two, three, four, five, six, seven, eight, nine, or ten linkers. In some embodiments, the guide nucleic acid comprises more than one linker. In some embodiments, at least two of the more than one linker are the same. In some embodiments, at least two of the more than one linker are not same.

In some embodiments, a linker comprises one to ten, one to seven, one to five, one to three, two to ten, two to eight, two to six, two to four, three to ten, three to seven, three to five, four to ten, four to eight, four to six, five to ten, five to seven, six to ten, six to eight, seven to ten, or eight to ten linked nucleotides. In some embodiments, the linker comprises one, two, three, four, five, six, seven, eight, nine, or ten linked nucleotides. In some embodiments, a linker comprises a nucleotide sequence of 5′-GAAA-3′.

In some embodiments, a guide nucleic acid comprises one or more linkers connecting one or more repeat sequences. In some embodiments, the guide nucleic acid comprises one or more linkers connecting one or more repeat sequences and one or more spacer sequences. In some embodiments, the guide nucleic acid comprises at least two repeat sequences connected by a linker.

### Intermediary Sequence

Guide nucleic acids described herein may comprise one or more intermediary sequences. In general, an intermediary sequence used in the present disclosure is not transactivated or transactivating. An intermediary sequence may also be referred to as an intermediary RNA, although it may comprise deoxyribonucleotides instead of or in addition to ribonucleotides, and/or edited bases. In general, the intermediary sequence non-covalently binds to an effector protein. In some embodiments, the intermediary sequence forms a secondary structure, for example in a cell, and an effector protein binds the secondary structure.

In some embodiments, a length of the intermediary RNA sequence is at least 30, 50, 70, 90, 110, 130, 150, 170, 190, or 210 linked nucleotides. In some embodiments, a length of the intermediary RNA sequence is not greater than 30, 50, 70, 90, 110, 130, 150, 170, 190, or 210 linked nucleotides. In some embodiments, the length of the intermediary RNA sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

An intermediary sequence may also comprise or form a secondary structure (e.g., one or more hairpin loops) that facilitates the binding of an effector protein to a guide nucleic acid and/or editing activity of an effector protein on a target nucleic acid (e.g., a hairpin region). An intermediary sequence may comprise from 5′ to 3′, a 5′ region, a hairpin region, and a 3′ region. In some embodiments, the 5′ region may hybridize to the 3′ region. In some embodiments, the 5′ region of the intermediary sequence does not hybridize to the 3′ region.

In some embodiments, the hairpin region may comprise a first sequence, a second sequence that is reverse complementary to the first sequence, and a stem-loop linking the first sequence and the second sequence. In some embodiments, an intermediary sequence comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, an intermediary sequence comprises a pseudoknot (e.g., a secondary structure comprising a stem at least partially hybridized to a second stem or half-stem secondary structure). An effector protein may interact with an intermediary sequence comprising a single stem region or multiple stem regions. In some embodiments, the nucleotide sequences of the multiple stem regions are identical to one another. In some embodiments, the nucleotide sequences of at least one of the multiple stem regions is not identical to those of the others. In some embodiments, an intermediary sequence comprises 1, 2, 3, 4, 5 or more stem regions.

### Handle Sequence

Guide nucleic acids described herein may comprise one or more handle sequences. In some embodiments, the handle sequence comprises an intermediary sequence. In such instances, at least a portion of an intermediary sequence non-covalently bonds with an effector protein. In some embodiments, the intermediary sequence is at the 3′-end of the handle sequence. In some embodiments, the intermediary sequence is at the 5′-end of the handle sequence. Additionally, or alternatively, in some embodiments, the handle sequence further comprises one or more of linkers and repeat sequences. In such instances, at least a portion of an intermediary sequence, or both of at least a portion of the intermediary sequence and at least a portion of repeat sequence, non-covalently interacts with an effector protein. In some embodiments, an intermediary sequence and repeat sequence are directly linked (e.g., covalently linked, such as through a phosphodiester bond). In some embodiments, the intermediary sequence and repeat sequence are linked by a suitable linker, examples of which are provided herein. In some embodiments, the linker comprises a sequence of 5′-GAAA-3′. In some embodiments, the intermediary sequence is 5′ to the repeat sequence. In some embodiments, the intermediary sequence is 5′ to the linker. In some embodiments, the intermediary sequence is 3′ to the repeat sequence. In some embodiments, the intermediary sequence is 3′ to the linker. In some embodiments, the repeat sequence is 3′ to the linker. In some embodiments, the repeat sequence is 5′ to the linker. In general, a single guide nucleic acid, also referred to as a single guide RNA (sgRNA), comprises a handle sequence comprising an intermediary sequence, and optionally one or more of a repeat sequence and a linker.

A handle sequence may comprise or form a secondary structure (e.g., one or more hairpin loops) that facilitates the binding of an effector protein to a guide nucleic acid and/or editing activity of an effector protein on a target nucleic acid (e.g., a hairpin region). In some embodiments, handle sequences comprise a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the handle sequence comprises a pseudoknot (e.g., a secondary structure comprising a stem at least partially hybridized to a second stem or half-stem secondary structure). An effector protein may recognize a handle sequence comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the handle sequence comprises at least 2, at least 3, at least 4, or at least 5 stem regions.

In some embodiments, a length of the handle sequence is at least 30, 50, 70, 90, 110, 130, 150, 170, 190, or 210 linked nucleotides. In some embodiments, a length of the handle sequence is not greater than 30, 50, 70, 90, 110, 130, 150, 170, 190, or 210 linked nucleotides. In some embodiments, the length of the handle sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

crRNA

In general, a crRNA comprises a spacer region that hybridizes to a target sequence of a target nucleic acid, and a repeat region that interacts with the effector protein. The spacer region may comprise complementarity with (e.g., hybridize to) a target sequence of a target nucleic acid. In some embodiments, the spacer region is 15-28 linked nucleosides in length. In some embodiments, the spacer region is 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleosides in length. In some embodiments, the spacer region is 18-24 linked nucleosides in length. In some embodiments, the spacer region is at least 15 linked nucleosides in length. In some embodiments, the spacer region is at least 16, 18, 20, or 22 linked nucleosides in length. In some embodiments, the spacer region comprises at least 10, 11, 12, 13, 14, 15, 16, 17, 18, 19, 20, 21, 22, 23, 24, 25, 26, 27, 28, 29, or 30 nucleotides. In some embodiments, the spacer region is at least 17 linked nucleosides in length. In some embodiments, the spacer region is at least 18 linked nucleosides in length. In some embodiments, the spacer region is at least 20 linked nucleosides in length. In some embodiments, the spacer region is at least 80%, at least 85%, at least 90%, at least 95% or 100% complementary to a target sequence of the target nucleic acid. In some embodiments, the spacer region is 100% complementary to the target sequence of the target nucleic acid. In some embodiments, the spacer region comprises at least 15 contiguous nucleobases that are complementary to the target nucleic acid. The repeat region may also be referred to as a “protein-binding segment.” Typically, the repeat region is adjacent to the spacer region. For example, a guide RNA that interacts with an effector protein comprises a repeat region that is 5′ of the spacer region.

It is understood that the sequence of a spacer region need not be 100% complementary to that of a target sequence of a target nucleic acid to hybridize or hybridize specifically to the target sequence. The guide nucleic acid may comprise at least one uracil between nucleic acid residues 5 to 20 of the spacer region that is not complementary to the corresponding nucleoside of the target sequence. The guide nucleic acid may comprise at least one uracil between nucleic acid residues 5 to 9, 10 to 14, or 15 to 20 of the spacer region that is not complementary to the corresponding nucleoside of the target sequence. In some embodiments, the region of the target nucleic acid that is complementary to the spacer region comprises an epigenetic modification or a post-transcriptional modification. In some embodiments, the epigenetic modification comprises an acetylation, methylation, or thiol modification.

In some embodiments, the guide RNA comprises a guide RNA described in the Examples herein. In some embodiments, the guide RNA comprises a base editor gRNA as shown in TABLE 5 or TABLE 7. In some embodiments, the guide RNA comprises any one of SEQ ID NOs: 532-538 and 540-541. In some embodiments, the guide RNA comprises any one of SEQ ID NOs: 773-779 and 781-782. In some embodiments, the guide RNA comprises repeat:spacer combinations of specific lengths, optimized for the target nucleic acid. In some embodiments, such guide RNAs comprise target nucleic acid FUT8-target 2. In some embodiments, such guide RNAs comprise a target nucleic acid comprising PDCD1-target 87. In some embodiments, such guide RNAs comprise a target nucleic acid comprising PDCD1-target 75. In some embodiments, such guide RNAs comprise a target nucleic acid B2M-target 2. In some embodiments, the guide nucleic acid is a guide RNA. In some embodiments, the guide nucleic acid comprises a sequence of any one of SEQ ID NOs: 650-652 combined with a sequence of any one of SEQ ID NOs: 653-676. In some embodiments, the guide nucleic acid comprises a sequence of any one of SEQ ID NOs: 783-785 combined with a sequence of any one of SEQ ID NOs: 786-809. In some embodiments, the guide nucleic acid comprises a sequence of any one of SEQ ID NOs: 532-538 and 540-541. In some embodiments, the guide nucleic acid comprises a sequence of any one of SEQ ID NOs: 773-779 and 781-782. In some embodiments, the guide nucleic acid comprises a spacer region of 18-20 nucleosides in length. In some embodiments, the guide nucleic acid comprises a spacer region of 18 linked nucleosides in length. In some embodiments, the guide nucleic acid comprises a sequence of any one of SEQ ID NOs: 650-652 combined with a sequence of any one of SEQ ID NOs: 656, 662, 668, or 674. In some embodiments, the guide nucleic acid comprises a sequence of any one of SEQ ID NOs: 783-785 combined with a sequence of any one of SEQ ID NOs: 789, 795, 801, or 807. In some embodiments, the guide nucleic acid comprises a spacer region of 19 linked nucleosides in length. In some embodiments, the guide nucleic acid comprises a spacer region of 20 linked nucleosides in length. In some embodiments, the guide nucleic acid comprises a sequence of any one of SEQ ID NOs: 650-652 combined with a sequence of any one of SEQ ID NOs: 657, 663, 669, or 675. In some embodiments, the guide nucleic acid comprises a sequence of any one of SEQ ID NOs: 783-785 combined with a sequence of any one of SEQ ID NOs: 790, 796, 802, or 808. In some embodiments, the gRNA comprises (SEQ ID NO: 651 combined with SEQ ID NO: 656). In some embodiments, the gRNA comprises (gRNA SEQ ID NO: 650 combined with SEQ ID NO: 656). In some embodiments, the gRNA comprises (gRNA SEQ ID NO: 651 combined with SEQ ID NO: 669). In some embodiments, the gRNA comprises (gRNA SEQ ID NO: 651 combined with SEQ ID NO: 668). In some embodiments, the gRNA comprises (SEQ ID NO: 784 combined with SEQ ID NO: 789). In some embodiments, the gRNA comprises (gRNA SEQ ID NO: 783 combined with SEQ ID NO: 789). In some embodiments, the gRNA comprises (gRNA SEQ ID NO: 784 combined with SEQ ID NO: 802). In some embodiments, the gRNA comprises (gRNA SEQ ID NO: 784 combined with SEQ ID NO: 802).

sgRNA

In some embodiments, a guide nucleic acid comprises a sgRNA. In some embodiments, a guide nucleic acid is a sgRNA. In some embodiments, a sgRNA comprises a first region (FR) and a second region (SR), wherein the FR comprises a handle sequence and the SR comprises a spacer sequence. In some embodiments, the handle sequence and the spacer sequences are directly connected to each other (e.g., covalent bond (phosphodiester bond)). In some embodiments, the handle sequence and the spacer sequence are connected by a linker.

In some embodiments, a sgRNA comprises one or more of one or more of a handle sequence, an intermediary sequence, a crRNA, a repeat sequence, a spacer sequence, a linker, or combinations thereof. For example, a sgRNA comprises a handle sequence and a spacer sequence; an intermediary sequence and an crRNA; an intermediary sequence, a repeat sequence and a spacer sequence; and the like.

In some embodiments, a sgRNA comprises an intermediary sequence and an crRNA. In some embodiments, an intermediary sequence is 5′ to a crRNA in an sgRNA. In some embodiments, a sgRNA comprises a linked intermediary sequence and crRNA. In some embodiments, an intermediary sequence and a crRNA are linked in an sgRNA directly (e.g., covalently linked, such as through a phosphodiester bond) In some embodiments, an intermediary sequence and a crRNA are linked in an sgRNA by any suitable linker, examples of which are provided herein.

In some embodiments, a sgRNA comprises a handle sequence and a spacer sequence. In some embodiments, a handle sequence is 5′ to a spacer sequence in an sgRNA. In some embodiments, a sgRNA comprises a linked handle sequence and spacer sequence. In some embodiments, a handle sequence and a spacer sequence are linked in an sgRNA directly (e.g., covalently linked, such as through a phosphodiester bond) In some embodiments, a handle sequence and a spacer sequence are linked in an sgRNA by any suitable linker, examples of which are provided herein.

In some embodiments, a sgRNA comprises an intermediary sequence, a repeat sequence, and a spacer sequence. In some embodiments, an intermediary sequence is 5′ to a repeat sequence in an sgRNA. In some embodiments, a sgRNA comprises a linked intermediary sequence and repeat sequence. In some embodiments, an intermediary sequence and a repeat sequence are linked in an sgRNA directly (e.g., covalently linked, such as through a phosphodiester bond) In some embodiments, an intermediary sequence and a repeat sequence are linked in an sgRNA by any suitable linker, examples of which are provided herein. In some embodiments, a repeat sequence is 5′ to a spacer sequence in an sgRNA. In some embodiments, a sgRNA comprises a linked repeat sequence and spacer sequence. In some embodiments, a repeat sequence and a spacer sequence are linked in an sgRNA directly (e.g., covalently linked, such as through a phosphodiester bond) In some embodiments, a repeat sequence and a spacer sequence are linked in an sgRNA by any suitable linker, examples of which are provided herein.

tracrRNA

In some embodiments, the guide RNA comprises a tracrRNA. The tracrRNA may be linked to a crRNA to form a composite gRNA. In some embodiments, the crRNA and the tracrRNA are provided as a single nucleic acid (e.g., covalently linked). In some embodiments, compositions comprise a tracrRNA that is separate from, but forms a complex with a crRNA to form a gRNA system. In some embodiments, the crRNA and the tracrRNA are separate polynucleotides.

A tracrRNA may comprise a repeat hybridization region and a hairpin region. The repeat hybridization region may hybridize to all or part of the sequence of the repeat of a crRNA. The repeat hybridization region may be positioned 3′ of the hairpin region. The hairpin region may comprise a first sequence, a second sequence that is reverse complementary to the first sequence, and a stem-loop linking the first sequence and the second sequence.

In some embodiments, tracrRNAs comprise a stem-loop structure comprising a stem region and a loop region. In some embodiments, the stem region is 4 to 8 linked nucleosides in length. In some embodiments, the stem region is 5 to 6 linked nucleosides in length. In some embodiments, the stem region is 4 to 5 linked nucleosides in length. In some embodiments, the tracrRNA comprises a pseudoknot (e.g., a secondary structure comprising a stem at least partially hybridized to a second stem or half-stem secondary structure). An effector protein may recognize a tracrRNA comprising multiple stem regions. In some embodiments, the amino acid sequences of the multiple stem regions are identical to one another. In some embodiments, the amino acid sequences of at least one of the multiple stem regions is not identical to those of the others. In some embodiments, the tracrRNA comprises at least 2, at least 3, at least 4, or at least 5 stem regions.

In some embodiments, the length of a tracrRNA is not greater than 50, 56, 68, 71, 73, 95, or 105 linked nucleosides. In some embodiments, the length of a tracrRNA is about 30 to about 120 linked nucleosides. In some embodiments, the length of a tracrRNA is about 50 to about 105, about 50 to about 95, about 50 to about 73, about 50 to about 71, about 50 to about 68, or about 50 to about 56 linked nucleosides. In some embodiments, the length of a tracrRNA is 56 to 105 linked nucleosides, from 56 to 105 linked nucleosides, 68 to 105 linked nucleosides, 71 to 105 linked nucleosides, 73 to 105 linked nucleosides, or 95 to 105 linked nucleosides. In some embodiments, the length of a tracrRNA is 40 to 60 nucleotides. In some embodiments, the length of a tracrRNA is 50, 56, 68, 71, 73, 95, or 105 linked nucleosides. In some embodiments, the length of a tracrRNA is 50 nucleotides.

An exemplary tracrRNA may comprise, from 5′ to 3′, a 5′ region, a hairpin region, a repeat hybridization region, and a 3′ region. In some embodiments, the 5′ region may hybridize to the 3′ region. In some embodiments, the 5′ region does not hybridize to the 3′ region. In some embodiments, the 3′ region is covalently linked to the crRNA (e.g., through a phosphodiester bond). In some embodiments, a tracrRNA may comprise an un-hybridized region at the 3′ end of the tracrRNA. The un-hybridized region may have a length of about 1, about 2, about 3, about 4, about 5, about 6, about 7, about 8, about 9, about 10, about 12, about 14, about 16, about 18, or about 20 linked nucleosides. In some embodiments, the length of the un-hybridized region is 0 to 20 linked nucleosides.

In some embodiments, the guide RNA does not comprise a tracrRNA. In some embodiments, an effector protein does not require a tracrRNA to locate and/or cleave a target nucleic acid. In some embodiments, the crRNA of the guide nucleic acid comprises a repeat region and a spacer region, wherein the repeat region binds to the effector protein and the spacer region hybridizes to a target sequence of the target nucleic acid. The repeat sequence of the crRNA may interact with an effector protein, allowing for the guide nucleic acid and the effector protein to form an RNP complex.

### III. Compositions

Disclosed herein are compositions comprising one or more effector proteins described herein or nucleic acids encoding the one or more effector proteins, one or more guide nucleic acids described herein or nucleic acids encoding the one or more guide nucleic acids described herein, or combinations thereof. In some embodiments, the guide nucleic acid comprises a first region and a second region. In some embodiments, the first region comprises one or more of a repeat sequence, a handle sequence, and an intermediary sequence. In some embodiments, one or more of the repeat sequence, handle sequence, and intermediary sequence interact with the one or more of the effector proteins. In some embodiments, the second region comprises one or more spacer sequences. In some embodiments, the one or more spacer sequences hybridize with target sequences of a target nucleic acid. In some embodiments, the compositions comprise one or more donor nucleic acids or nucleic acids encoding the one or more donor nucleic acids. In some embodiments, the compositions edit a target nucleic acid in a cell or a subject. In some embodiments, the compositions edit a target nucleic acid or the expression thereof in a cell, in a tissue, in an organ, in vitro, in vivo, or ex vivo. In some embodiments, the compositions edit a target nucleic acid in a sample comprising the target nucleic.

In some embodiments, compositions described herein comprise plasmids described herein, viral vectors described herein, non-viral vectors described herein, or combinations thereof. In some embodiments, compositions described herein comprise the viral vectors. In some embodiments, compositions described herein comprise an AAV. In some embodiments, compositions described herein comprise liposomes (e.g., cationic lipids or neutral lipids), dendrimers, lipid nanoparticle (LNP), or cell-penetrating peptides. In some embodiments, compositions described herein comprise an LNP.

Disclosed herein is a composition comprising: a fusion protein comprising at least one of the fusion partners as described herein. In some embodiments, at least one of the fusion partners comprise an amino acid sequence that is at least 70%, at least 75%, at least 80%, at least 85%, at least 90%, at least 95%, at least 97%, at least 98%, at least 99% or 100% identical to any one of the sequences described in TABLE 2. In some embodiments, the composition further comprises at least one guide nucleic acid that comprises a first sequence that hybridizes to a target sequence of a double stranded DNA molecule, and a second sequence that binds to an effector protein.

As another example, disclosed herein is a composition that comprises: (a) an effector protein or a nucleic acid encoding the effector protein; and (b) a base editor or a nucleic acid encoding the base editor, wherein the base editor is optionally directly or indirectly linked to the effector protein.

As another example, disclosed herein is a composition that comprises: (a) an effector protein or a nucleic acid encoding the effector protein; and (b) a prime editing enzyme or a nucleic acid encoding the prime editing enzyme, wherein the prime editing enzyme is optionally directly or indirectly linked to the effector protein.

As another example, disclosed herein is a composition that comprises a CRISPRi fusion comprising: (a) an effector protein or a nucleic acid encoding the effector protein; and (b) a fusion partner or a nucleic acid encoding the fusion partner, wherein the fusion partner is optionally directly or indirectly linked to the effector protein.

As another example, disclosed herein is a composition that comprises a CRISPRa fusion comprising: (a) an effector protein or a nucleic acid encoding the effector protein; and (b) a fusion partner or a nucleic acid encoding the fusion partner, wherein the fusion partner is optionally directly or indirectly linked to the effector protein.

As another example, disclosed herein is a composition that comprises: (a) an effector protein or a nucleic acid encoding the effector protein; and (b) an RNA splicing factor or a nucleic acid encoding the RNA splicing factor, wherein the RNA splicing factor is optionally directly or indirectly linked to the effector protein.

As another example, disclosed herein is a composition that comprises: (a) an effector protein or a nucleic acid encoding the effector protein; and (b) a recombinase or a nucleic acid encoding the recombinase, wherein the recombinase is optionally directly or indirectly linked to the effector protein.

As a specific example of a composition, disclosed herein is a composition comprising: a fusion protein comprising a fusion partner; and at least one guide nucleic acid that comprises a first sequence that hybridizes to a target sequence of a double stranded DNA molecule, and a second sequence that binds to an effector protein, wherein the fusion protein comprises a DNA alkylating fusion partner as described herein. In some embodiments, the composition further comprises a repair inhibitor fusion partner as described herein. As another example, disclosed herein is a composition that comprises: (a) an effector protein or a nucleic acid encoding the effector protein; and (b) a DNA alkylating fusion partner or a nucleic acid encoding the DNA alkylating fusion partner, wherein the DNA alkylating fusion protein is optionally directly or indirectly linked to the effector protein. In some embodiments, the composition further comprises a repair inhibitor fusion partner or a nucleic acid encoding the repair inhibitor fusion partner.

As another specific example of a composition, disclosed herein is a composition comprising: a fusion protein; and at least one guide nucleic acid that comprises a first sequence that hybridizes to a target sequence of a double stranded DNA molecule, and a second sequence that binds to an effector protein, wherein the fusion protein comprises a plurality of fusion partner, wherein the plurality of fusion partner comprises methyl transferase fusion partner as described herein, and the deaminase fusion partner as described herein. In some embodiments, the plurality of fusion partner further comprises a thymine DNA glycosylase inhibitor fusion partner. As another example, disclosed herein is a composition that comprises: (a) an effector protein or a nucleic acid encoding the effector protein; and (b) a deaminase fusion partner or a nucleic acid encoding the deaminase fusion partner, wherein the deaminase fusion partner is optionally directly or indirectly linked to the effector protein. In some embodiments, the composition further comprises a methyl transferase fusion partner or a nucleic acid encoding the methyl transferase fusion partner, wherein the methyl transferase fusion partner is optionally directly or indirectly linked to the effector protein. In some embodiments, the deaminase fusion partner deaminates methyl cytosine residues of the non-target strand of the double stranded DNA molecule at a greater rate than cytosine residues of the non-target strand of the double stranded DNA molecule. In some embodiments, the guide nucleic acid comprises a guanine residue at its 5′ end. In some embodiments, the composition further comprises a thymine DNA glycosylase inhibitor fusion partner or a nucleic acid encoding the thymine DNA glycosylase inhibitor fusion partner.

As yet another specific example of a composition, disclosed herein is a composition comprising: (a) an effector protein or a nucleic acid encoding the effector protein; and (b) a terminal deoxynucleotidyl transferase (TdT) fusion partner or a nucleic acid encoding the TdT fusion partner, wherein the TdT fusion partner is optionally directly or indirectly linked to the effector protein. In some embodiments, the guide nucleic acid does not comprise the at least 5 nucleic acids. In some embodiments, the composition further comprises a second guide RNA, wherein the second guide RNA recognizes a PAM sequence that is different from the PAM sequence recognized by the guide RNA.

As yet another specific example of a composition, disclosed herein is a composition comprising: a) an effector protein or a nucleic acid encoding the effector protein; and (b) an RNA pseudouridylation fusion partner or a nucleic acid encoding the RNA pseudouridylation fusion partner, wherein the RNA pseudouridylation fusion partner is optionally directly or indirectly linked to the effector protein.

As still yet another specific example of a composition, disclosed herein is a composition comprising: a) an effector protein or a nucleic acid encoding the effector protein; and (b) a fusion partner or a nucleic acid encoding the fusion partner, wherein the fusion partner is optionally directly or indirectly linked to the effector protein, wherein the fusion partner comprises an N-alkylating fusion partner, an oxidizing fusion partner, a cytosine deaminating fusion partner, an apurinic or apyrimidinic site generating fusion partner, a ribonucleotide reductase fusion partner, or combinations thereof.

### Pharmaceutical Compositions and Modes of Administration

Disclosed herein, in some aspects, are pharmaceutical compositions for modifying a target nucleic acid in a cell or a subject, comprising any one of the fusion effector proteins described herein, or a nucleic acid encoding any one of the fusion effector proteins described herein. Also disclosed herein are pharmaceutical compositions for modifying the expression of a target nucleic acid in a cell or a subject, comprising any one of the fusion effector proteins described herein. In some embodiments, pharmaceutical compositions comprise a guide nucleic acid. Pharmaceutical compositions may be used to modify a target nucleic acid or the expression thereof in a cell in vitro, in vivo or ex vivo.

In some embodiments, pharmaceutical compositions comprise one or more nucleic acids encoding an effector protein, fusion effector protein, fusion partner, a guide nucleic acid, or a combination thereof; and a pharmaceutically acceptable carrier or diluent. The effector protein, fusion effector protein, fusion partner protein, or combination thereof may be any one of those described herein. The one or more nucleic acids may comprise a plasmid. The one or more nucleic acids may comprise a nucleic acid expression vector. The one or more nucleic acids may comprise a viral vector. In some embodiments, the viral vector is a lentiviral vector. In some embodiments, the vector is an adeno-associated viral (AAV) vector. In some embodiments, compositions, including pharmaceutical compositions, comprise a viral vector encoding a fusion effector protein and a guide nucleic acid, wherein at least a portion of the guide nucleic acid binds to the effector protein of the fusion effector protein.

In some embodiments, pharmaceutical compositions comprise a virus comprising a viral vector encoding a fusion effector protein, an effector protein, a fusion partner, a guide nucleic acid, or a combination thereof, and a pharmaceutically acceptable carrier or diluent. The virus may be a lentivirus. The virus may be an adenovirus. The virus may be a non-replicating virus. The virus may be an AAV. The viral vector may be a retroviral vector. Retroviral vectors may include gamma-retroviral vectors such as vectors derived from the Moloney Murine Keukemia Virus (MoMLV, MMLV, MuLV, or MLV) or the Murine Steam cell Virus (MSCV) genome. Retroviral vectors may include lentiviral vectors such as those derived from the human immunodeficiency virus (HIV) genome. In some embodiments, the viral vector is a chimeric viral vector, comprising viral portions from two or more viruses. In some embodiments, the viral vector is a recombinant viral vector.

In some embodiments, the viral vector is an AAV. The AAV may be any AAV known in the art. In some embodiments, the viral vector corresponds to a virus of a specific serotype. In some examples, the serotype is selected from an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV11 serotype, and an AAV12 serotype. In some embodiments the AAV vector is a recombinant vector, a hybrid AAV vector, a chimeric AAV vector, a self-complementary AAV (scAAV) vector, a single-stranded AAV or any combination thereof. scAAV genomes are generally known in the art and contain both DNA strands which can anneal together to form double-stranded DNA.

In some embodiments, methods of producing delivery vectors herein comprise packaging an engineered guide disclosed herein in an AAV vector. In some examples, methods of producing the delivery vectors described herein comprise, (a) introducing into a cell: (i) a polynucleotide encoding any engineered guide disclosed herein; and (ii) a viral genome comprising a Replication (Rep) gene and Capsid (Cap) gene that encodes a wild-type AAV capsid protein or modified version thereof, (b) expressing in the cell the wild-type AAV capsid protein or modified version thereof, (c) assembling an AAV particle; and (d) packaging the polynucleotide encoding the engineered polynucleotide in the AAV particle, thereby generating an AAV delivery vector. In some embodiments, an engineered guide disclosed herein, promoters, stuffer sequences, and any combination thereof may be packaged in the AAV vector. In some examples, the AAV vector can package 1, 2, 3, 4, or 5 copies of the engineered guide. In some embodiments, the recombinant vectors comprise one or more inverted terminal repeats and the inverted terminal repeats comprise a 5′ inverted terminal repeat, a 3′ inverted terminal repeat, and a mutated inverted terminal repeat. In some examples, the mutated terminal repeat lacks a terminal resolution site.

In some embodiments, a hybrid AAV vector is produced by transcapsidation, e.g., packaging an inverted terminal repeat (ITR) from a first serotype into a capsid of a second serotype, wherein the first and second serotypes may be not the same. In some examples, the Rep gene and ITR from a first AAV serotype (e.g., AAV2) may be used in a capsid from a second AAV serotype (e.g., AAV9), wherein the first and second AAV serotypes may be not the same. As a non-limiting example, a hybrid AAV serotype comprising the AAV2 ITRs and AAV9 capsid protein may be indicated AAV2/9. In some examples, the hybrid AAV delivery vector comprises an AAV2/1, AAV2/2, AAV 2/4, AAV2/5, AAV2/8, or AAV2/9 vector.

In some embodiments, the AAV vector may be a chimeric AAV vector. In some embodiments, the chimeric AAV vector comprises an exogenous amino acid or an amino acid substitution, or capsid proteins from two or more serotypes. In some examples, a chimeric AAV vector may be genetically engineered to increase transduction efficiency, selectivity, or a combination thereof.

In some examples, the delivery vector may be a eukaryotic vector, a prokaryotic vector (e.g., a bacterial vector) a viral vector, or any combination thereof. In some embodiments, the delivery vehicle may be a non-viral vector. In some embodiments, the delivery vehicle may be a plasmid. In some embodiments, the plasmid comprises DNA. In some embodiments, the plasmid comprises RNA. In some examples, the plasmid comprises circular double-stranded DNA. In some examples, the plasmid may be linear. In some examples, the plasmid comprises one or more genes of interest and one or more regulatory elements. In some examples, the plasmid comprises a bacterial backbone containing an origin of replication and an antibiotic resistance gene or other selectable marker for plasmid amplification in bacteria. In some examples, the plasmid may be a minicircle plasmid. In some examples, the plasmid contains one or more genes that provide a selective marker to induce a target cell to retain the plasmid. In some examples, the plasmid may be formulated for delivery through injection by a needle carrying syringe. In some examples, the plasmid may be formulated for delivery via electroporation. In some examples, the plasmids may be engineered through synthetic or other suitable means known in the art. For example, in some embodiments, the genetic elements may be assembled by restriction digest of the desired genetic sequence from a donor plasmid or organism to produce ends of the DNA which may then be readily ligated to another genetic sequence.

In some embodiments, the vector is a non-viral vector, and a physical method or a chemical method is employed for delivery into the somatic cell. Exemplary physical methods include electroporation, gene gun, sonoporation, magnetofection, or hydrodynamic delivery. Exemplary chemical methods include delivery of the recombinant polynucleotide via liposomes such as, cationic lipids or neutral lipids; dendrimers; nanoparticles; or cell-penetrating peptides.

In some embodiments, a fusion effector protein as described herein is inserted into a vector. In some embodiments, the vector optionally comprises one or more promoters, enhancers, ribosome binding sites, RNA splice sites, polyadenylation sites, a replication origin, and/or transcriptional terminator sequences.

In general, plasmids and vectors described herein comprise at least one promoter. In some embodiments, the promoters are constitutive promoters. In other embodiments, the promoters are inducible promoters. In additional embodiments, the promoters are prokaryotic promoters (e.g., drive expression of a gene in a prokaryotic cell). In some embodiments, the promoters are eukaryotic promoters, (e.g., drive expression of a gene in a eukaryotic cell). Exemplary promoters include, but are not limited to, CMV, EF1a, SV40, PGK1, Ubc, human beta actin, CAG, TRE, UAS, Ac5, polyhedron, CaMKIIa, GAL1-10, TEF1, GDS, ADH1, CaMV35S, Ubi, H1, U6, CaMV35S, SV40, CMV, and HSV TK promoter. In some embodiments, the promoter is CMV. In some embodiments, the promoter is EF1a. In some embodiments, the promoter is ubiquitin.

In some embodiments, vectors are bicistronic or polycistronic vector (e.g., having or involving two or more loci responsible for generating a protein) having an internal ribosome entry site (IRES) is for translation initiation in a cap-independent manner.

In some embodiments, vectors comprise an enhancer. Enhancers are nucleotide sequences that have the effect of enhancing promoter activity. In some embodiments, enhancers augment transcription regardless of the orientation of their sequence. In some embodiments, enhancers activate transcription from a distance of several kilo base pairs. Furthermore, enhancers are located optionally upstream or downstream of a gene region to be transcribed, and/or located within the gene, to activate the transcription. Exemplary enhancers include, but are not limited to, WPRE; CMV enhancers; the R-U5′ segment in LTR of HTLV-I (Mol. Cell. Biol., Vol. 8(1), p. 466-472, 1988); SV40 enhancer; the intron sequence between exons 2 and 3 of rabbit β-globin (Proc. Natl. Acad. Sci. USA., Vol. 78(3), p. 1527-31, 1981); and the genome region of human growth hormone (J Immunol., Vol. 155(3), p. 1286-95, 1995).

Pharmaceutical compositions described herein may comprise a salt. In some embodiments, the salt is a sodium salt. In some embodiments, the salt is a potassium salt. In some embodiments, the salt is a magnesium salt. In some embodiments, the salt is NaCl. In some embodiments, the salt is KNO₃. In some embodiments, the salt is Mg²+SO₄²⁻.

Non-limiting examples of pharmaceutically acceptable carriers and diluents suitable for the pharmaceutical compositions disclosed herein include buffers (e.g., neutral buffered saline, phosphate buffered saline); carbohydrates (e.g., glucose, mannose, sucrose, dextran, mannitol); polypeptides or amino acids (e.g., glycine); antioxidants; chelating agents (e.g., EDTA, glutathione); adjuvants (e.g., aluminum hydroxide); surfactants (Polysorbate 80, Polysorbate 20, or Pluronic F68); glycerol; sorbitol; mannitol; polyethyleneglycol; and preservatives.

In some embodiments, pharmaceutical compositions are in the form of a solution (e.g., a liquid). The solution may be formulated for injection, e.g., intravenous or subcutaneous injection. In some embodiments, the pH of the solution is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some embodiments, the pH of the solution is less than 7. In some embodiments, the pH is greater than 7.

Disclosed herein is a pharmaceutical composition comprising a fusion partner or a nucleic acid encoding the fusion partner, a fusion protein comprising the fusion partner or a nucleic acid encoding the fusion protein, a composition comprising the fusion partner; or a system comprising the fusion partner; and a pharmaceutically acceptable carrier or diluent. In some embodiments, the fusion partner can be any of the fusion partners as described herein. In some embodiments, the fusion partner comprises an amino acid sequence that is at least 70%, at least 75%, at least 80%, at least 85%, at least 90%, at least 95%, at least 97%, at least 98%, at least 99% or 100% identical to any one of the sequences described in TABLE 2.

### IV. Methods

Provided herein are methods of editing target nucleic acids. In general, editing refers to modifying the nucleobase sequence of a target nucleic acid. Also provided herein are methods of modulating the expression of a target nucleic acid. Fusion effector proteins and systems described herein may be used for such methods. Methods of editing a target nucleic acid may comprise one or more of cleaving the target nucleic acid, deleting one or more nucleotides of the target nucleic acid, inserting one or more nucleotides into the target nucleic acid, modifying one or more nucleotides of the target nucleic acid. Methods of modulating expression of target nucleic acids may comprise modifying the target nucleic acid or a protein associated with the target nucleic acid, e.g., a histone.

In some embodiments, methods comprise contacting a target nucleic acid with a fusion effector protein described herein. The fusion effector protein may comprise an effector protein described in TABLE 1 or a catalytically inactive variant thereof. The effector protein may comprise an amino acid sequence that is at least 70%, at least 75%, at least 80%, at least 85%, at least 90%, at least 95%, at least 97%, at least 98%, at least 99% or 100% identical to a sequence described in TABLE 1. The fusion effector protein may comprise a fusion partner protein described in TABLE 2. The fusion partner protein may comprise an amino acid sequence that is at least 70%, at least 75%, at least 80%, at least 85%, at least 90%, at least 95%, at least 97%, at least 98%, at least 99% or 100% identical to a sequence described in TABLE 2.

The target nucleic acid may be in a cell or a subject. The cell may be a dividing cell. The cell may be a terminally differentiated cell. In some embodiments, the target nucleic acid is a gene. In some embodiments, the gene comprises a mutation. In some embodiments, the mutation or the gene is associated with a disease. In some embodiments, the mutation is an autosomal dominant mutation. In some embodiments, the mutation is a premature stop codon. In some embodiments, the mutation is a dominant negative mutation. In some embodiments, the mutation is a SNP. In some embodiments, the mutation is a loss of function mutation. Non-limiting examples of diseases associated with genetic mutations are cystic fibrosis, Duchenne muscular dystrophy, β-thalassemia, and Usher syndrome.

In some embodiments, methods comprise base editing. In general, base editing comprises contacting a target nucleic acid with an enzyme, such as a deaminase, thereby changing a nucleobase of the target nucleic acid to a different nucleobase. In some embodiments, the nucleobase of the target nucleic acid is adenine (A) and the method comprises changing A to guanine (G). In some embodiments, the nucleobase of the target nucleic acid is cytosine (C) and the method comprises changing C to thymine (T). In some embodiments, the nucleobase of the target nucleic acid is C and the method comprises changing C to G. In some embodiments, the nucleobase of the target nucleic acid is A and the method comprises changing A to G.

Methods of editing may introduce a nucleobase change in a target nucleic acid relative to a corresponding wildtype or mutant nucleobase sequence. Editing may remove or correct a disease-causing mutation in a nucleic acid sequence, e.g., to produce a corresponding wildtype nucleobase sequence. Editing may remove/correct point mutations, deletions, null mutations, or tissue-specific mutations in a target nucleic acid. Editing may be used to generate gene knock-out, gene knock-in, gene editing, gene tagging, or a combination thereof. Methods of the disclosure may be targeted to any locus in a genome of a cell.

Methods of editing a target nucleic acid or modulating the expression of a target nucleic acid may be performed in vivo. Methods of editing a target nucleic acid or modulating the expression of a target nucleic acid may be performed in vitro. For example, a plasmid may be modified in vitro using a composition described herein and introduced into a cell or organism. Methods of editing a target nucleic acid or modulating the expression of a target nucleic acid may be performed ex vivo. For example, methods may comprise obtaining a cell from a subject, modifying a target nucleic acid in the cell with methods described herein, and returning the cell to the subject. Methods of editing performed ex vivo may be particularly advantageous to produce CAR T-cells.

Methods of editing a target nucleic acid or modulating the expression of a target nucleic acid described herein may be employed to generate a genetically modified cell. The cell may be a eukaryotic cell (e.g., a mammalian cell) or a prokaryotic cell (e.g., an archaeal cell). The cell may be a human cell. The cell may be a T cell. The cell may be a hematopoietic stem cell. The cell may be a bone marrow derived cell (e.g., a white blood cell or blood cell progenitor). Generating a genetically modified cell may comprise contacting a target cell with a fusion effector protein and a guide nucleic acid. Contacting may comprise electroporation, acoustic poration, optoporation, viral vector-based delivery, iTOP, nanoparticle delivery (e.g., lipid or gold nanoparticle delivery), cell-penetrating peptide (CPP) delivery, DNA nanostructure delivery, or any combination thereof. In some embodiments, the nanoparticle delivery comprises lipid nanoparticle delivery or gold nanoparticle delivery. In some embodiments, the nanoparticle delivery comprises lipid nanoparticle delivery. In some embodiments, the nanoparticle delivery comprises gold nanoparticle delivery.

Methods may comprise cell line engineering. Generally, cell line engineering comprises modifying a pre-existing cell (e.g., naturally-occurring or engineered) or pre-existing cell line to produce a novel cell line or modified cell line. The novel or modified cell line may be useful for production of a protein of interest. In some embodiments, the pre-existing cell line is a Chinese hamster ovary cell line (CHO), a human embryonic kidney cell line (HEK), a cell line derived from a cancer cell, a cell line derived from lymphocytes. Non-limiting examples of pre-existing cell lines include: C8161, CCRF-CEM, MOLT, mIMCD-3, NHDF, HeLa-S3, Huhl, Huh4, Huh7, HUVEC, HASMC, HEKn, HEKa, MiaPaCell, Panc1, PC-3, TF1, CTLL-2, CIR, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, and YAR.

### Methods of Modifying a Target Nucleic Acid

Disclosed herein is a method of modifying a target nucleic acid or the expression thereof, the method comprising: contacting the target nucleic acid with a fusion partner disclosed herein, a fusion protein disclosed herein, a pharmaceutical composition comprising a fusion protein disclosed herein, or a system comprising a fusion protein disclosed herein, thereby modifying the target nucleic acid or the expression thereof. In some embodiments, the fusion partner comprises a plurality of fusion partner. In some embodiments, the target nucleic acid is in a cell. In some embodiments, the cell is in vitro. In some embodiments, the cell is ex vivo. In some embodiments, the cell is in vivo. In some embodiments, the cell is a prokaryotic cell. In some embodiments, the cell is a eukaryotic cell. In some embodiments, the cell is a mammalian cell. In some embodiments, the cell is an immune cell, where optionally the immune cell is a T cell.

In some embodiments, a target nucleic acid comprises a portion or a specific region of a nucleic acid from a genomic locus, any DNA amplicon of, a reverse transcribed mRNA, or a cDNA from a gene described herein. In some embodiments, the target nucleic acid is an amplicon of at least a portion of a gene. Non-limiting examples of genes are AAVS1, ABCA4, ABCB11, ABCC8, ABCD1, ABCG5, ABCG8, ACAD9, ACADM, ACADVL, ACAT1, ACOX1, ACSF3, ADA, ADAMTS2, ADGRG1, AGA, AGL, AGPS, AGXT, AHI1, AIRE, ALDH3A2, ALDOB, ALG6, ALK, ALKBH5, ALMS1, ALPL, AMRC9, AMT, ANAPC10, ANAPC11, ANGPTL3, ANGPTL4, APC, Apo(α), APOCIII, APOEε4, APOL1, APP, AQP2, AR, ARFRP1, ARG1, ARH, ARL13B, ARL6, ARSA, ARSB, ASL, ASNS, ASPA, ASS1, ATM, ATP6V1B1, ATP7A, ATP7B, ATRX ATXN1, ATXN10, ATXN2, ATXN3, ATXN7, ATXN8OS, AXIN1, AXIN2, B2M, BACE-1, BAK1, BAP1, BARD1, BAX2, BBS1, BBS10, BBS12, BBS2, BCKDHA, BCKDHB, BCL2L2, BCS1L, BEST1, Betaglobin gene, BLM, BMPR1A, BRAF, BRAFV600E, BRCA1, BRCA2, BRIP1, BSND, C9orf72, CA4, CACNA1A, CAH1, CAPN3, CASR, CBS, CCNB1 CC2D2A, CCR5, CD1, CD2, CD3, CD3D, CD3Z, CD4, CD5, CD6, CD7, CD8A, CD8B, CD9, CD14, CD18, CD19, CD21, CD22, CD23, CD27, CD28, CD30, CD33, CD34, CD36, CD38, CD40, CD40L, CD44, CD46, CD47, CD48, CD52, CD55, CD57, CD58, CD59, CD68, CD69, CD72, CD73, CD74, CD79A, CD80, CD81, CD83, CD84, CD86, CD90, CD93, CD96, CD99, CD100, CD123, CD160, CD163, CD164, CD164L2, CD166, CD200, CD204, CD207, CD209, CD226, CD244, CD247, CD274, CD276, CD300, CD320, CDC73, CDH1, CDH23, CDK11, CDK4, CDKN1A, CDKN1B, CDKN1C, CDKN2A, CDKN2B, CEBPA, CELA3B, CEP290, CERKL, CFB, CFTR, CHCHD10, CHEK2, CHM, CHRNE, CIDEB, CIITA, CLN3, CLN5, CLN6, CLN8, CLRN1, CLTA, CMT1A, CNBP, CNGB1, CNGB3, COL1A1, COL1A2, COL27A1, COL4A3, COL4A4, COL4A5, COL7AM, CPS1, CPT1A, CPT2, CRB1, CREBBP, CRX, CRYAA, CTNNA1, CTNNB1, CTNND2, CTNS, CTSK, CXCL12, CYBA, CYBB, CYP11B1, CYP11B2, CYP17A1, CYP19A1, CYP21A2, CYP27A1, DBT, DCC, DCLRE1C, DERL2, DFNA36, DFNB31, DGAT2, DHCR7, DHDDS, DICER1, DIS3L2, DLD, DMD, DMPK, DNAH5, DNAI1, DNAI2, DNM2, DNMT1, DPC4, DYSF, EDA, EDN3, EDNRB, EGFR, EIF2B5, EMC2, EMC3, EMD, EMX1, EN1, EPCAM, ERCC6, ERCC8, ESCO2, ETFA, ETFDH, ETHE1, EVC, EVC2, EYS, F5, F9, FXI, FAH, FAM161A, FANCA, FANCB, FANCC, FANCD1, FANCD2, FANCE, FANCF, FANCG, FANCI, FANCJ, FANCL, FANCM, FANCN, FANCP, FANCS, FBN1, FGF14, FGFR2, FGFR3, FGA, FGB, FGG, FH, FHL1, FIX, FKRP, FKTN, FLCN, FMR1, FOXP3, FSCN2, FSHD1, FUS, FUT8, FVIII, FXII, FXN, G6PC, GAA, GALC, GALK1, GALT, GAMT, GATA2, GATA-4, GBA, GBE1, GCDH, GCGR, GDNF, GFAP, GFM1, GHR, GJB1, GJB2, GLA, GLB1, GLDC, GLE1, GNE, GNPTAB, GNPTG, GNS, GPAM, GPC3, GPR98, GREM1, GRHPR, GRIN2B, H2AFX H2AX, HADHA, HAX1, HBA1, HBA2, HBB, HBV cccDNA, HER2, HEXA, HEXB, HFE, HGSNAT, HLCS, HMGCL, HAO1, HOGA1, HOXB13, HPRPF3, HPRT1, HPS1, HPS3, HRAS, HRD1, HSD3B2, HSDI7B4, HSDI7B13, HTT, HUS1, HYAL1, HYLS1, IDS, IDUA, IFITM5, IKBKAP, IL2RG, IL7R, IMPDH1, INPP5E, IRF4, ITGB2, ITPR1, IVD, JAG1, JAK1, JAK3, KCNC3, KCND3, KCNJ11, KLKB1, KLHL7, KRAS, LAMA2, LAMA3, LAMB3, LAMC2, LCA5, LDHA, LDLR, LDLRAP1, LHX3, LIFR, LIPA, LMNA, LOR, LOXHD1, LPA, LPL, LRAT, LRP6, LRPPRC, LRRK2, MADR2, MAN2B1, MAPT, MARC1, MAX, MCM6, MCOLN1, MECP2, MED17, MEFV, MEN1, MERTK, MESP2, MET, METex14, MFN2, MFSD8, MIA3, MITF, MKL2, MKS1, MLC1, MLH1, MLH3, MvAA, MMAB, MMACHC, MMADHC, MMD, MPI, MPL, MPV17, MSH2, MSH3, MSH6, MTHFD1L, MTHFR, MTMJ, MTRR, MTTP, MUT, MUTYH, MYC, MYH7, MYO7A, NAGLU, NAGS, NBN, NDRG1, NDUFAF5, NDUFS6, NEB, NF1, NF2, NKX2-5, NOG, NOTCH1, NOTCH2, NPC1, NPC2, NPHP1, NPHS1, NPHS2, NRAS, NR2E3, NTHL1, NTRK, NTRK1, OAT, OCT4, OFD1, OPA3, OTC, PAH, PALB2, PAQR8, PAX3, PC, PCCA, PCCB, PCDH15, PCSK9, PD1, PDCD1, PDE6B, PDGFRA, PDHA1, PDHB, PEX1, PEX10, PEX12, PEX13, PEX14, PEX16, PEX19, PEX2, PEX26, PEX3, PEX5, PEX6, PEX7, PFKM, PHGDH, PHOX2B, PKD1, PKD2, PKHD1, PKK, PLEKHG4, PMM2, PMP22, PMS1, PMS2, PNPLA3, POLD1, POLE, POMGNT1, POT1, POU5F1, PPM1A, PPP2R2B, PPT1, PRCD, PRKAG2, PRKARIA, PRKCG, PRNP, PROM1, PROP1, PRPF31, PRPF8, PRPH2, PRPS1, PSAP, PSD3, PSD95, PSEN1, PSEN2, PSRC1, PTCH1, PTEN, PTS, PUS1, PYGM, RAB23, RAD50, RAD51C, RAD51D, RAG1, RAG2, RAPSN, RARS2, RB1, RDH12, RECQL4, RET, RHO, RICTOR, RMRP, ROS1, RP1, RP2, RPE65, RPGR, RPGRIP1L, RPL32P3, RS1, RTCA, RTEL1, RUNX1, SACS, SAMHDI, SCNIA, SCN2A, SDHA, SDHAF2, SDHB, SDHC, SDHD, SEL1L, SEPSECS, SERPINA1, SERPINC1, SERPING1, SGCA, SGCB, SGCG, SGSH, SIRT1, SLC12A3, SLC12A6, SLC17A5, SLC22A5, SLC25A13, SLC25A15, SLC26A2, SLC26A4, SLC35A3, SLC35B4, SLC37A4, SLC39A4, SLC4A11, SLC6A8, SLC7A7, SMAD3, SMAD4, SMARCA4, SMARCAL1, SMARCB1, SMARCE1, SMN1, SMPD1, SNAI2, SNCA, SNRNP200, SOD1, SOX10, SPARA7, SPTBN2, STAR, STAT3, STKI1, SUFU, SUMF1, SYNE1, SYNE2, SYS1, TARDBP, TAT, TBK1, TBP, TCIRG1, TCTN3, TECPR2, TERC, TERT, TFR2, TGFBR2, TGM1, TH, TLE3, TMEM127, TMEM138, TMEM216, TMEM43, TMEM67, TMPRSS6, TOP1, TOPORS, TP53, TPP1, TRAC, TRMU, TSC1, TSC2, TSFM, TSPAN14, TTBK2, TTC8, TTPA, TTR, TULP1, TYMP, UBE2G2, UBE2J1, UBE3A, USH1C, USH1G, USH2A, VEGF, VHL, VPS13A, VPS13B, VPS35, VPS45, VRK1, VSX2, VWF, WAS, WDR19, WDR48, WNT10A, WRN, WS2B, WS2C, WT1, XPA, XPC, XPF, XRCC3, YAP1, ZAC1, ZEB1, ZFYVE26, and ZNF423. Nucleic acid sequences of target nucleic acids and/or corresponding genes are readily available in public databases as known and used in the art. In some embodiments, the target nucleic acid is selected from any one of the target nucleic acids described herein. In some embodiments, the target nucleic acid comprises one or more target sequences. In some embodiments, the one or more target sequence is within any one of the target nucleic acids described herein.

In some embodiments, the target nucleic acid modified by the methods described herein is a target double stranded DNA molecule and a fusion protein comprising at least one of the fusion partners as described herein. In some embodiments, the at least one of the fusion partners comprise an amino acid sequence that is at least 70%, at least 75%, at least 80%, at least 85%, at least 90%, at least 95%, at least 97%, at least 98%, at least 99% or 100% identical to any one of the sequences described in TABLE 2.

In some embodiments, a target nucleic acid is modified by the methods described herein comprise a fusion protein comprising a base editor as described herein, wherein the target nucleic is an RNA, a single strand of DNA or both strands of dsDNA.

In some embodiments, a target nucleic acid is modified by the methods described herein comprise a fusion protein comprising a prime editing enzyme as described herein, wherein the target nucleic is a dsDNA.

In some embodiments, a target nucleic acid is modified by the methods described herein comprise a fusion protein comprising a recombinase domain as described herein.

In some embodiments, a target nucleic acid is modified by the methods described herein comprise a fusion protein comprising a fusion partner, wherein the fusion partner is a DNA alkylating fusion partner as described herein, and wherein the target nucleic acid is a target double stranded DNA molecule. In such embodiments, the fusion partner can comprise a plurality of fusion partner, wherein the plurality of fusion partner comprises a DNA alkylating fusion partner as described herein, and a repair inhibitor fusion partner as described herein. In such embodiments, the contacting is sufficient to produce in the target double stranded DNA molecule: (a) an O⁶-guanine through O-alkylation of a guanine in the target DNA molecule, (b) an O⁴-thymine through O-alkylation of a thymine in the target DNA molecule, or (c) an N¹-guanine through N-alkylation of a guanine in the target DNA molecule.

In some embodiments, a target nucleic acid is modified by the methods described herein comprise a fusion protein comprising a plurality of fusion partners, wherein the plurality of fusion partners comprise a deaminase fusion partner as described herein, and an engineered methyl transferase fusion partner as described herein. In some embodiments, the plurality of fusion partners further comprises a thymine DNA glycosylase inhibitor fusion partner.

In some embodiments, a target nucleic acid is modified by the methods described herein comprise a fusion protein comprising a terminal deoxynucleotidyl transferase (TdT) fusion partner as described herein. In such embodiments, the TdT fusion partner, upon contact with a DNA molecule, a DNA molecule comprising an overhang is generated. In some embodiments, the DNA molecule comprising the overhang is ligated by the microhomology-mediated end joining (MMEJ) pathway in a cell, thereby inserting a nucleotide sequence into the DNA molecule in the cell.

In some embodiments, a target nucleic acid is modified by the methods described herein comprise a fusion protein comprising an RNA pseudouridylation fusion partner as described herein. In such embodiments, the target nucleic acid is an mRNA transcript. In some embodiments, contacting the RNA pseudouridylation fusion partner to the mRNA transcript causes pseudouridylation of a uridine present in the nonsense codon. In some embodiments, the pseudouridylation suppresses the nonsense codon associated, and thereby modifying the mRNA transcript.

In some embodiments, a target nucleic acid is modified by the methods described herein comprise a fusion protein comprising an N-alkylating fusion partner, an oxidizing fusion partner, a cytosine deaminating fusion partner, an apurinic or apyrimidinic site generating fusion partner, a ribonucleotide reductase fusion partner, or combinations thereof. In such embodiments, the fusion partner when contacted with the target nucleic acid: (a) modifies a nucleobase of the target nucleotide, thereby generating a modified nucleobase at the target nucleotide; (b) site-specifically excises the modified nucleobase, thereby generating an apurinic or apyrimidinic site at the target nucleotide; and (c) attaches a new nucleobase at the apurinic or apyrimidinic site; thereby performing targeted nucleotide substitution of the target nucleotide. In some embodiments, the method further comprises contacting the target nucleic acid with NTPs. In some embodiments, the NTP is ATP, and wherein the new nucleobase attached to the apurinic or apyrimidinic site is an adenine. In some embodiments, the NTP is TTP, and wherein the new nucleobase attached to the apurinic or apyrimidinic site is a thymine. In some embodiments, the NTP is GTP, and wherein the new nucleobase attached to the apurinic or apyrimidinic site is a guanine. In some embodiments, the NTP is CTP, and wherein the new nucleobase attached to the apurinic or apyrimidinic site is a cytosine.

### Methods of Treating a Disease or Disorder

Described herein are methods for treating a disease in a subject by editing a target nucleic acid associated with a gene or expression of a gene related to the disease. In some embodiments, the methods comprise methods of editing nucleic acid described herein.

In some embodiments, methods for treating a disease in a subject comprise administration of a composition(s) or component(s) of a system described herein. In some embodiments, the composition(s) or component(s) of the system comprises use of a recombinant nucleic acid (DNA or RNA), administered for the purpose to edit a nucleic acid. In some embodiments, the composition or component of the system comprises use of a vector to introduce a functional gene or transgene. In some embodiments, vectors comprise nonviral vectors, including cationic polymers, cationic lipids, or bio-responsive polymers. In some embodiments, the bio-responsive polymer exploits chemical-physical properties of the endosomal environment (e.g., pH) to preferentially release the genetic material in the intracellular space. In some embodiments, vectors comprise viral vectors, including retroviruses, adenoviruses, adeno-associated viruses, and herpes simplex viruses. In some embodiments, the vector comprises a replication-defective viral vector, comprising an insertion of a therapeutic gene inserted in genes essential to the lytic cycle, preventing the virus from replicating and exerting cytotoxic effects. Methods of gene therapy that are applicable to the compositions and systems described herein are described in more detail in Ingusci et al., “Gene Therapy Tools for Brain Diseases”, Front. Pharmacol. 10:724 (2019), which is hereby incorporated by reference in its entirety.

Disclosed herein is a method of treating a genetic disease or disorder associated with a mutation in a target DNA molecule in a subject in need thereof, the method comprising administering to the subject a fusion protein disclosed herein, a composition comprising a fusion protein disclosed herein, a pharmaceutical composition comprising a fusion protein disclosed herein, or a system comprising a fusion protein disclosed herein. In such embodiments, the administering is sufficient to modify or repair the mutation, thereby treating the genetic disease or disorder. In some embodiments, the mutation comprises a single nucleotide polymorphism (SNP). In some embodiments, the mutation comprises a frameshift mutation.

In some embodiments, the methods described herein comprise administering at least one of the fusion proteins as described herein, wherein the administering the at least one of the fusion proteins is sufficient to produce a modification in the target nucleic acid. In some embodiments, the fusion protein comprises a fusion partner, wherein the fusion partner comprises an amino acid sequence that is at least 70%, at least 75%, at least 80%, at least 85%, at least 90%, at least 95%, at least 97%, at least 98%, at least 99% or 100% identical to any one of the sequences described in TABLE 2. Also, described herein are use of fusion proteins as described herein for treating a disease or disorder described herein according to the methods described herein.

In some embodiments, methods of treating a disease or disorder described herein comprise administering a fusion protein comprising a base editor as described herein. Also described herein is a use of the fusion protein comprising the base editor in treating the disease or disorder by administering the fusion protein. Also described herein is the fusion protein comprising the base editor for use in treating the disease or disorder by administering the fusion protein. In some embodiments, the administering is sufficient to modify an RNA, a single strand of DNA or both strands of dsDNA.

In some embodiments, methods of treating a disease or disorder described herein comprise administering a fusion protein comprising a prime editing enzyme as described herein. Also described herein is a use of the fusion protein comprising the prime editing enzyme in treating the disease or disorder by administering the fusion protein. Also described herein is the fusion protein comprising the prime editing enzyme for use in treating the disease or disorder by administering the fusion protein. In some embodiments, the administering is sufficient to modify a dsDNA.

In some embodiments, methods of treating a disease or disorder described herein comprise administering a CRISPRi fusion as described herein. Also described herein is a use of the CRISPRi fusion in treating the disease or disorder by administering the CRISPRi fusion. Also described herein is the CRISPRi fusion for use in treating the disease or disorder by administering the CRISPRi fusion. In some embodiments, the administering is sufficient to directly and/or indirectly provides for decreased transcription and/or translation of a target nucleic acid.

In some embodiments, methods of treating a disease or disorder described herein comprise administering a CRISPRa fusion as described herein. Also described herein is a use of the CRISPRa fusion in treating the disease or disorder by administering the CRISPRa fusion. Also described herein is the CRISPRa fusion for use in treating the disease or disorder by administering the CRISPRa fusion. In some embodiments, the administering is sufficient to directly and/or indirectly provides for increased transcription and/or translation of a target nucleic acid.

In some embodiments, methods of treating a disease or disorder described herein comprise administering a fusion protein comprising an RNA splicing factor as described herein. Also described herein is a use of the fusion protein comprising the RNA splicing factor in treating the disease or disorder by administering the fusion protein. Also described herein is the fusion protein comprising the RNA splicing factor for use in treating the disease or disorder by administering the fusion protein. In some embodiments, the RNA splicing factor is capable of providing modular organization.

In some embodiments, methods of treating a disease or disorder described herein comprise administering a fusion protein comprising a recombinase domain as described herein. Also described herein is a use of the fusion protein comprising the recombinase domain in treating a disease or disorder by administering the fusion protein. Also described herein is the fusion protein comprising the recombinase domain for use in treating the disease or disorder by administering the fusion protein. In some embodiments, the recombinase domain is capable of interacting with a target nucleic acid in a site-specific manner.

In some embodiments, methods of treating a disease or disorder described herein comprise a fusion protein comprising a DNA alkylating fusion partner as described herein. Also described herein is a use of the fusion protein comprising the DNA alkylating fusion partner in treating the disease or disorder by administering the fusion protein. Also described herein is the fusion protein comprising the DNA alkylating fusion partner for use in treating the disease or disorder by administering the fusion protein. In some embodiments, the administering is sufficient to produce in the target DNA molecule associated with the mutation: (a) an O⁶-guanine through O-alkylation of a guanine in the target DNA molecule, (b) an O⁴-thymine through O-alkylation of a thymine in the target DNA molecule, or (c) an N¹-guanine through N-alkylation of a guanine in the target DNA molecule. In such embodiments, the administering is sufficient to repair the mutation by producing the O-alkylation or N-alkylation. In such embodiments, the disease or disorder can be a genetic disease or disorder.

In some embodiments, methods of treating a disease or disorder described herein comprise administering a fusion protein comprising a plurality of fusion partners, wherein the plurality of fusion partners comprise a deaminase fusion partner as described herein, and a methyl transferase fusion partner as described herein. In such embodiments, the disease or disorder can be a genetic disease or disorder. In some embodiments, the plurality of fusion partner further comprises a thymine DNA glycosylase inhibitor fusion partner. Also described herein is a use of the fusion protein comprising at least two of the fusion partners for treating the disease or disorder, wherein the at least two of the fusion partners are selected from the deaminase fusion partner, the methyl transferase fusion partner, and the thymine DNA glycosylase inhibitor fusion partner. Also described herein is the fusion protein comprising at least two of the fusion partners for treating the disease or disorder, wherein the at least two of the fusion partners are selected from the deaminase fusion partner, the methyl transferase fusion partner, and the thymine DNA glycosylase inhibitor fusion partner for use in treating the disease or disorder by administering the fusion protein.

In some embodiments, methods of treating a disease or disorder described herein comprise administering a fusion protein comprising a terminal deoxynucleotidyl transferase (TdT) fusion partner as described herein. Also described herein is a use of the fusion protein comprising the TdT fusion partner for treating the disease or disorder. Also described herein is the fusion protein comprising the TdT fusion partner for use in treating the disease or disorder by administering the fusion protein. In some embodiments, the TdT fusion partner, upon contact with a DNA molecule, a DNA molecule comprising an overhang is generated. In some embodiments, the overhang in the subject is ligated by the non-homologous end joining (NHEJ) pathway, thereby correcting a frameshift mutation in the subject.

In some embodiments, methods of treating a disease or disorder described herein comprise administering a fusion protein comprising a RNA pseudouridylation fusion partner as described herein. Also described herein is a use of the fusion protein comprising the RNA pseudouridylation fusion partner in treating the disease or disorder by administering the fusion protein. Also described herein is the fusion protein comprising the RNA pseudouridylation fusion partner for use in treating the disease or disorder by administering the fusion protein. In some embodiments, the RNA pseudouridylation fusion partner performs pseudouridylation of a uridine present in the nonsense codon, thereby suppressing the nonsense codon associated with the disease or disorder. In some embodiments, the disease or disorder is cystic fibrosis, hemophilia, sickle cell disease, or Duchenne muscular dystrophy.

In some embodiments, methods of treating a disease or disorder described herein comprise administering a fusion protein comprising an N-alkylating fusion partner, an oxidizing fusion partner, a cytosine deaminating fusion partner, an apurinic or apyrimidinic site generating fusion partner, a ribonucleotide reductase fusion partner, or combinations thereof. Also described herein is a use of the fusion protein comprising the N-alkylating fusion partner, the oxidizing fusion partner, the cytosine deaminating fusion partner, the apurinic or apyrimidinic site generating fusion partner, the ribonucleotide reductase fusion partner, or combinations thereof in treating the disease or disorder by administering the fusion protein. Also described herein is the fusion protein comprising the N-alkylating fusion partner, the oxidizing fusion partner, the cytosine deaminating fusion partner, the apurinic or apyrimidinic site generating fusion partner, the ribonucleotide reductase fusion partner, or combinations thereof for use in treating the disease or disorder by administering the fusion protein.

In some embodiments, treating, preventing, or inhibiting disease or disorder in a subject may comprise contacting a target nucleic acid associated with a particular ailment with a composition described herein. In some aspects, the methods of treating, preventing, or inhibiting a disease or disorder may involve removing, editing, modifying, replacing, transposing, or affecting the regulation of a genomic sequence of a patient in need thereof. In some embodiments, the methods of treating, preventing, or inhibiting a disease or disorder may involve modulating gene expression.

Described herein are compositions and methods for treating a disease in a subject by editing a target nucleic acid associated with a gene or expression of a gene related to the disease. In some embodiments, methods comprise administering a composition or cell described herein to a subject. By way of non-limiting example, the disease may be a cancer, an ophthalmological disorder, a neurological disorder, a neurodegenerative disease, a blood disorder, or a metabolic disorder, or a combination thereof. The disease may be an inherited disorder, also referred to as a genetic disorder. The disease may be the result of an infection or associated with an infection.

The compositions and methods described herein may be used to treat, prevent, or inhibit a disease or syndrome in a subject. In some embodiments, the disease is a liver disease, a lung disease, an eye disease, or a muscle disease. Exemplary diseases and syndromes include but are not limited to 11-hydroxylase deficiency; 17,20-desmolase deficiency; 17-hydroxylase deficiency; 3-hydroxyisobutyrate aciduria; 3-hydroxysteroid dehydrogenase deficiency; 46,XY gonadal dysgenesis; AAA syndrome; ABCA3 deficiency; ABCC8-associated hyperinsulinism; aceruloplasminemia; acromegaly; achondrogenesis type 2; acral peeling skin syndrome; acrodermatitis enteropathica; adrenocortical micronodular hyperplasia; adrenoleukodystrophies; adrenomyeloneuropathies; Aicardi-Goutieres syndrome; Alagille disease (also called Alagille Syndrome); Alexander Disease; Alpers syndrome; alpha-1 antitrypsin deficiency (AATD); alpha-mannosidosis; Alstrom syndrome; Alzheimer's disease; amebic dysentery; amelogenesis imperfecta; amish type microcephaly; amyotrophic lateral sclerosis (ALS); anaplastic large cell lymphoma; anauxetic dysplasia; androgen insensitivity syndrome; angiopathic thrombosis; antiphospholipid syndrome; Antley-Bixler syndrome; APECED; Apert syndrome; aplasia of lacrimal and salivary glands; arginase-1 deficiency; argininosuccinic aciduria; argininemia; arrhythmogenic right ventricular dysplasia; Arts syndrome; ARVD2; arylsulfatase deficiency type metachromatic leukodystrophy; ataxia telangiectasia; atherosclerotic cardiovascular disease; autoimmune lymphoproliferative syndrome; autoimmune polyglandular syndrome type 1; autosomal dominant anhidrotic ectodermal dysplasia; autosomal dominant deafness; autosomal dominant polycystic kidney disease; autosomal recessive microtia; autosomal recessive renal glucosuria; autosomal visceral heterotaxy; babesiosis; balantidial dysentery; Bardet-Biedl syndrome; Bartter syndrome; basal cell nevus syndrome; Batten disease; benign recurrent intrahepatic cholestasis; beta-mannosidosis; β-thalassemia; Bethlem myopathy; Blackfan-Diamond anemia; bleeding disorder (coagulation); blepharophimosis; Byler disease; C syndrome; CADASIL; calcific aortic stenosis; calcification of joints and arteries; carbamoyl phosphate synthetase I deficiency; cardiofaciocutaneous syndrome; Carney triad; carnitine palmitoyltransferase deficiencies; cartilage-hair hypoplasia; cblC type of combined methylmalonic aciduria; CD18 deficiency; CD3Z-associated primary T-cell immunodeficiency; CD40L deficiency; CDAGS syndrome; CDG1A; CDG1B; CDG1M; CDG2C; CEDNIK syndrome; central core disease; centronuclear myopathy; cerebral capillary malformation; cerebrooculofacioskeletal syndrome type 4; cerebrooculogacioskeletal syndrome; cerebrotendinous xanthomatosis; Chaga's Disease; Charcot Marie Tooth Disease; cherubism; CHILD syndrome; chronic granulomatous disease; chronic recurrent multifocal osteomyelitis; cirrhosis; citrin deficiency; citrullinemia type I; citrullinemia type II; classic hemochromatosis; CNPPB syndrome; cobalamin C disease; Cockayne syndrome; coenzyme Q10 deficiency; Coffin-Lowry syndrome; Cohen syndrome; combined deficiency of coagulation factors V; common variable immune deficiency 3; complement hyperactivation; complete androgen insentivity; cone rod dystrophies; conformational diseases; congenital adrenal hyperplasia; congenital bile adid synthesis defect type 1; congenital bile adid synthesis defect type 2; congenital defect in bile acid synthesis type; congenital erythropoietic porphyria; congenital generalized osteosclerosis; congenital hyperplasia (CAH); Cornelia de Lange syndrome; coronary heart disease; Cousin syndrome; Cowden disease; COX deficiency; Cri du chat syndrome; Crigler-Najjar disease; Crigler-Najjar syndrome type 1; Crisponi syndrome; Crouzon syndrome; Currarino syndrome; Curth-Macklin type ichthyosis hystrix; cutis laxa; cystic fibrosis; cystinosis; d-2-hydroxyglutaric aciduria; DDP syndrome; Dejerine-Sottas disease; Denys-Drash syndrome; Dercum disease; desmin cardiomyopathy; desmin myopathy; DGUOK-associated mitochondrial DNA depletion; diabetes Type I; diabetes Type II; disorders of glutamate metabolism; distal spinal muscular atrophy type 5; DNA repair diseases; dominant optic atrophy; Doyne honeycomb retinal dystrophy; Dravet Syndrome; Duchenne muscular dystrophy; dyskeratosis congenita; Ehlers-Danlos syndrome type 4; Ehlers-Danlos syndromes; Elejalde disease; Ellis-van Creveld disease; Emery-Dreifuss muscular dystrophies; encephalomyopathic mtDNA depletion syndrome; encephalitis; enzymatic diseases; EPCAM-associated congenital tufting enteropathy; epidermolysis bullosa with pyloric atresia; epilepsy; fabry disease; facioscapulohumeral muscular dystrophy; Factor V Leiden thrombophilia; Faisalabad histiocytosis; familial atypical mycobacteriosis; familial capillary malformation-arteriovenous; Familial Creutzfeld-Jakob disease; familial esophageal achalasia; familial glomuvenous malformation; familial hemophagocytic lymphohistiocytosis; familial mediterranean fever; familial megacalyces; familial schwannomatosis; familial spina bifida; familial splenic asplenia/hypoplasia; familial thrombotic thrombocytopenic purpura; Fanconi disease (Fanconi anemia); Feingold syndrome; FENIB; fibrodysplasia ossificans progressiva; FKTN; Fragile X syndrome; Francois-Neetens fleck corneal dystrophy; Frasier syndrome; Friedreich's ataxia; FTDP-17; Fuchs corneal dystrophy; fucosidosis; G6PD deficiency; galactosialidosis; Galloway syndrome; Gardner syndrome; Gaucher disease; Gitelman syndrome; GLUT1 deficiency; GM2-Gangliosidoses (e.g., Tay Sachs Disease, Sandhoff Disease) glycogen storage disease type 1b; glycogen storage disease type 2; glycogen storage disease type 3; glycogen storage disease type 4; glycogen storage disease type 9a; glycogen storage diseases; GM1-gangliosidosis; Greenberg syndrome; Greig cephalopolysyndactyly syndrome; hair genetic diseases; hairy cell leukemia; HANAC syndrome; harlequin type ichtyosis congenita; HDR syndrome; hearing loss; hemochromatosis type 3; hemochromatosis type 4; hemolytic anemia; hemolytic uremic syndrome; hemophilia A; hemophilia B; hereditary angioedema type 3; hereditary angioedemas; hereditary hemorrhagic telangiectasia; hereditary hyperfibrinogenemia; hereditary intraosseous vascular malformation; hereditary leiomyomatosis and renal cell cancer; hereditary neuralgic amyotrophy; hereditary sensory and autonomic neuropathy type; Hermansky-Pudlak disease; HHH syndrome; HHT2; hidrotic ectodermal dysplasia type 1; hidrotic ectodermal dysplasias; histiocytic sarcoma; HNF4A-associated hyperinsulinism; HNPCC; homozygous familial hypercholesterolemia; human immunodeficiency with microcephaly; Human monkeypox (MPX); human papilloma virus (HPV) infection; Huntington's disease; hyper-IgD syndrome; hyperinsulinism-hyperammonemia syndrome; hypercholesterolemia; hypertrophy of the retinal pigment epithelium; hypochondrogenesis; hypohidrotic ectodermal dysplasia; ICF syndrome; idiopathic congenital intestinal pseudo-obstruction; immunodeficiency 13; immunodeficiency 17; immunodeficiency 25; immunodeficiency with hyper-IgM type 1; immunodeficiency with hyper-IgM type 3; immunodeficiency with hyper-IgM type 4; immunodeficiency with hyper-IgM type 5; immunoglobulin alpha deficiency; inborn errors of thyroid metabolism; infantile myofibromatosis; infantile visceral myopathy; infantile X-linked spinal muscular atrophy; intrahepatic cholestasis of pregnancy; IPEX syndrome; IRAK4 deficiency; isolated congenital asplenia; Jeune syndrome; Johanson-Blizzard syndrome; Joubert syndrome; JP-HHT syndrome; juvenile hemochromatosis; juvenile hyalin fibromatosis; juvenile nephronophthisis; Kabuki mask syndrome; Kallmann syndromes; Kartagener syndrome; KCNJ11-associated hyperinsulinism; Kearns-Sayre syndrome; Kostmann disease; Kozlowski type of spondylometaphyseal dysplasia; Krabbe disease; LADD syndrome; late infantile-onset neuronal ceroid lipofuscinosis; LCK deficiency; LDHCP syndrome; Leber Congenital Amaurosis Teyp 10; Legius syndrome; Leigh syndrome; lethal congenital contracture syndrome 2; lethal congenital contracture syndromes; lethal contractural syndrome type 3; lethal neonatal CPT deficiency type 2; lethal osteosclerotic bone dysplasia; leukocyte adhesion deficiency; Li Fraumeni syndrome; LIG4 syndrome; lipodystrophy; lissencephaly type 1; lissencephaly type 3; Loeys-Dietz syndrome; low phospholipid-associated cholelithiasis; Lynch Syndrome; lysinuric protein intolerance; a lysosomal storage disease (e.g., Hunter syndrome, Hurler syndrome); macular dystrophy; Maffucci syndrome; Majeed syndrome; mannose-binding protein deficiency; mantle cell lymphoma; Marfan disease; Marshall syndrome; MASA syndrome; mastocytosis; MCAD deficiency; McCune-Albright syndrome; MCKD2; Meckel syndrome; MECP2 Duplication Syndrome; Meesmann corneal dystrophy; megacystis-microcolon-intestinal hypoperistalsis; megaloblastic anemia type 1; MEHMO; MELAS; Melnick-Needles syndrome; MEN2s; meningitis; Menkes disease; metachromatic leukodystrophies; methymalonic acidemia due to transcobalamin receptor defect; methylmalonic acidurias; methylvalonic aciduria; microcoria-congenital nephrosis syndrome; microvillous atrophy; migraine; mitochondrial neurogastrointestinal encephalomyopathy; monilethrix; monosomy X; mosaic trisomy 9 syndrome; Mowat-Wilson syndrome; mucolipidosis type 2; mucolipidosis type Ma; mucolipidosis type IV; mucopolysaccharidoses; mucopolysaccharidosis type 3A; mucopolysaccharidosis type 3C; mucopolysaccharidosis type 4B; multiminicore disease; multiple acyl-CoA dehydrogenation deficiency; multiple cutaneous and mucosal venous malformations; multiple endocrine neoplasia type 1; multiple sulfatase deficiency; mycosis fungoides; myotonic dystrophy; NAIC; nail-patella syndrome; nemaline myopathies; neonatal diabetes mellitus; neonatal surfactant deficiency; nephronophtisis; Netherton disease; neurofibromatoses; neurofibromatosis type 1; Niemann-Pick disease type A; Niemann-Pick disease type B; Niemann-Pick disease type C; NKX2E; non-alcoholic fatty liver disease (NAFLD); non-alcoholic steatohepatitis (NASH); Noonan syndrome; North American Indian childhood cirrhosis; NROB1 duplication-associated DSD; ocular genetic diseases; oculo-auricular syndrome; OLEDAID; oligomeganephronia; oligomeganephronic renal hypolasia; Ollier disease; Opitz-Kaveggia syndrome; ornithine transcarbamylase deficiency (OTCD); orofaciodigital syndrome type 1; orofaciodigital syndrome type 2; osseous Paget disease; osteogenesis imperfecta; otopalatodigital syndrome type 2; OXPHOS diseases; palmoplantar hyperkeratosis; panlobar nephroblastomatosis; Parkes-Weber syndrome; Parkinson's disease; partial deletion of 21q22.2-q22.3; Pearson syndrome; Pelizaeus-Merzbacher disease; Pendred syndrome; pentalogy of Cantrell; peroxisomal acyl-CoA-oxidase deficiency; Peutz-Jeghers syndrome; Pfeiffer syndrome; Pierson syndrome; pigmented nodular adrenocortical disease; pipecolic acidemia; Pitt-Hopkins syndrome; plasmalogens deficiency; platelet glycoprotein IV deficiency; pleuropulmonary blastoma and cystic nephroma; polycystic kidney disease; polycystic ovarian disease; polycystic lipomembranous osteodysplasia; Pompe disease; including infantile onset Pompe disease (IOPD) and late onset Pompe disease (LOPD); porphyrias; PRKAG2 cardiac syndrome; premature ovarian failure; primary erythermalgia; primary hemochromatoses; primary hyperoxaluria; progressive familial intrahepatic cholestasis; propionic acidemia; protein-losing enteropathy; pyruvate decarboxylase deficiency; RAPADILTINO syndrome; renal cystinosis; retinitis pigmentosa; Rett Syndrome; rhabdoid tumor predisposition syndrome; Rieger syndrome; ring chromosome 4; Roberts syndrome; Robinow-Sorauf syndrome; Rothmund-Thomson syndrome; severe combined immunodeficiency disorder (SCID); Saethre-Chotzen syndrome; Sandhoff disease; SC phocomelia syndrome; SCAS; Schinzel phocomelia syndrome; severe hypertriglyceridemia; short rib-polydactyly syndrome type 1; short rib-polydactyly syndrome type 4; short-rib polydactyly syndrome type 2; short-rib polydactyly syndrome type 3; Shwachman disease; Shwachman-Diamond disease; sickle cell anemia; Silver-Russell syndrome; Simpson-Golabi-Behmel syndrome; Smith-Lemli-Opitz syndrome; SPG7-associated hereditary spastic paraplegia; spherocytosis; spinocerebellar ataxia; spinal muscular atrophy; split-hand/foot malformation with long bone deficiencies; spondylocostal dysostosis; sporadic amyotrophic lateral sclerosis; sporadic visceral myopathy with inclusion bodies; storage diseases; Stargardt macular dystrophy; STRA6-associated syndrome; stroke; Tay-Sachs disease; thanatophoric dysplasia; thrombophilia due to antithrombin III deficiency; thyroid metabolism diseases; Tourette syndrome; transcarbamylase deficiency; transthyretin-associated amyloidosis; trisomy 13; trisomy 22; trisomy 2p syndrome; tuberous sclerosis; tufting enteropathy; urea cycle diseases; Usher Syndrome; Van Den Ende-Gupta syndrome; Van der Woude syndrome; variegated mosaic aneuploidy syndrome; VLCAD deficiency; von Hippel-Lindau disease; von Willebrand disease; Waardenburg syndrome; WAGR syndrome; Walker-Warburg syndrome; Werner syndrome; Wilson's disease; Wiskott-Aldrich Syndrome; Wolcott-Rallison syndrome; Wolfram syndrome; X-linked agammaglobulinemia; X-linked chronic idiopathic intestinal pseudo-obstruction; X-linked cleft palate with ankyloglossia; X-linked dominant chondrodysplasia punctata; X-linked ectodermal dysplasia; X-linked Emery-Dreifuss muscular dystrophy; X-linked lissencephaly; X-linked lymphoproliferative disease; X-linked visceral heterotaxy; xanthinuria type 1; xanthinuria type 2; xeroderma pigmentosum; XPV; and Zellweger disease.

In some embodiments, compositions and methods edit at least one gene associated with a disease described herein or the expression thereof. In some embodiments, the disease is Alzheimer's disease and the gene is selected from APP, BACE-1, PSD95, MAPT, PSEN1, PSEN2, and APOEε4. In some embodiments, the disease is Parkinson's disease and the gene is selected from SNCA, GDNF, and LRRK2. In some embodiments, the disease comprises Centronuclear myopathy and the gene is DNM2. In some embodiments, the disease is Huntington's disease and the gene is HTT. In some embodiments, the disease is Alpha-1 antitrypsin deficiency (AATD) and the gene is SERPINA1. In some embodiments, the disease is amyotrophic lateral sclerosis (ALS) and the gene is selected from SOD1, FUS, C90RF72, ATXAN2, TARDBP, and CHCHD10. In some embodiments, the disease comprises Alexander Disease and the gene is GFAP. In some embodiments, the disease comprises anaplastic large cell lymphoma and the gene is CD30. In some embodiments, the disease comprises Angelman Syndrome and the gene is UBE3A. In some embodiments, the disease comprises calcific aortic stenosis and the gene is Apo(a). In some embodiments, the disease comprises CD3Z-associated primary T-cell immunodeficiency and the gene is CD3Z or CD247. In some embodiments, the disease comprises CD18 deficiency and the gene is ITGB2. In some embodiments, the disease comprises CD40L deficiency and the gene is CD40L. In some embodiments, the disease is congenital adrenal hyperplasia and the gene is CAHL. In some embodiments, the disease comprises CNS trauma and the gene is VEGF. In some embodiments, the disease comprises coronary heart disease and the gene is selected from FGA, FGB, and FGG. In some embodiments, the disease comprises MECP2 Duplication syndrome and Rett syndrome and the gene is MECP2. In some embodiments, the disease comprises a bleeding disorder (coagulation) and the gene is FXI. In some embodiments, the disease comprises fragile X syndrome and the gene is FMRL. In some embodiments, the disease comprises Fuchs corneal dystrophy and the gene is selected from ZEB1, SLC4A11, and LOXHD1. In some embodiments, the disease comprises GM2-Gangliosidoses (e.g., Tay Sachs Disease, Sandhoff disease) and the gene is selected from HEXA and HEXB. In some embodiments, the disease comprises Hearing loss disorders and the gene is DFNA36. In some embodiments, the disease is Pompe disease, including infantile onset Pompe disease (IOPD) and late onset Pompe disease (LOPD) and the gene is GAA. In some embodiments, the disease is Retinitis pigmentosa and the gene is selected from PDE6B, RHO, RP1, RP2, RPGR, PRPH2, IMPDH1, PRPF31, CRB1, PRPF8, TULP1, CA4, HPRPF3, ABCA4, EYS, CERKL, FSCN2, TOPORS, SNRNP200, PRCD, NR2E3, MERTK, USH2A, PROM1, KLHL7, CNGB1, TTC8, ARL6, DHDDS, BEST1, LRAT, SPARA7, CRX, CLRN1, RPE65, and WDR19. In some embodiments, the disease comprises Leber Congenital Amaurosis Type 10 and the gene is CEP290. In some embodiments, the disease is cardiovascular disease and/or lipodystrophies and the gene is selected from ABCG5, ABCG8, AGT, ANGPTL3, APOCIII, APOA1, APOL1, ARH, CDKN2B, CFB, CXCL12, FXT, FXII, GATA-4, MIA3, MKL2, MTHFD1L, MYH7, NKX2-5, NOTCH1, PKK, PCSK9, PSRC1, SMAD3, and TTR. In some embodiments, the disease is cardiovascular disease and/or lipodystrophies and the gene is ANGPTL3. In some embodiments, the disease is cardiovascular disease and/or lipodystrophies and the gene is PCSK9. In some embodiments, the disease is cardiovascular disease and/or lipodystrophies and the gene is TTR. In some embodiments, the disease is severe hypertriglyceridemia (SHTG) and the gene is APOCIII or ANGPTL4. In some embodiments, the disease comprises acromegaly and the gene is GHR. In some embodiments, the disease comprises acute myeloid leukemia and the gene is CD22. In some embodiments, the disease is diabetes and the gene is GCGR. In some embodiments, the disease is NAFLD/NASH and the gene is selected from HSDI7B13, PSD3, GPAM, CIDEB, DGAT2 and PNPLA3. In some embodiments, the disease is NASH/cirrhosis and the gene is MARC1. In some embodiments, the disease is cancer and the gene is selected from STAT3, YAP1, FOXP3, AR (Prostate cancer), and IRF4 (multiple myeloma). In some embodiments, the disease is cystic fibrosis and the gene is CFTR. In some embodiments, the disease is Duchenne muscular dystrophy and the gene is DMD. In some embodiments, the disease is ornithine transcarbamylase deficiency (OTCD) and the gene is OTC. In some embodiments, the disease is congenital adrenal hyperplasia (CAH) and the gene is CYP21A2. In some embodiments, the disease is atherosclerotic cardiovascular disease (ASCVD) and the gene is LPA. In some embodiments, the disease is hepatitis B virus infection (CHB) and the gene is HBV covalently closed circular DNA (cccDNA). In some embodiments, the disease is citrullinemia type I and the gene is ASS1. In some embodiments, the disease is citrullinemia type I and the gene is SLC25A13. In some embodiments, the disease is citrullinemia type I and the gene is ASS1. In some embodiments, the disease is arginase-1 deficiency and the gene is ARG1. In some embodiments, the disease is carbamoyl phosphate synthetase I deficiency and the gene is CPS1. In some embodiments, the disease is argininosuccinic aciduria and the gene is ASL. In some embodiments, the disease comprises angioedema and the gene is PKK. In some embodiments, the disease comprises thalassemia and the gene is TMPRSS6. In some embodiments, the disease comprises achondroplasia and the gene is FGFR3. In some embodiments, the disease comprises Cri du chat syndrome and the gene is selected from CTNND2. In some embodiments, the disease comprises sickle cell anemia and the gene is Beta globin gene. In some embodiments, the disease comprises Alagille Syndrome and the gene is selected from JAG1 and NOTCH2. In some embodiments, the disease comprises Charcot-Marie-Tooth disease and the gene is selected from PMP22 and MFN2. In some embodiments, the disease comprises Crouzon syndrome and the gene is selected from FGFR2, FGFR3, and FGFR3. In some embodiments, the disease comprises Dravet Syndrome and the gene is selected from SCN1A and SCN2A. In some embodiments, the disease comprises Emery-Dreifuss syndrome and the gene is selected from EMD, LMNA, SYNE1, SYNE2, FHL1, and TMEM43. In some embodiments, the disease comprises Factor V Leiden thrombophilia and the gene is F5. In some embodiments, the disease is fabry disease and the gene is GLA. In some embodiments, the disease is facioscapulohumeral muscular dystrophy and the gene is FSHD1. In some embodiments, the disease comprises Fanconi anemia and the gene is selected from FANCA, FANCB, FANCC, FANCD1, FANCD2, FANCE, FANCF, FANCG, FANCI, FANCJ, FANCL, FANCM, FANCN, FANCP, FANCS, RAD51C, and XPF. In some embodiments, the disease comprises Familial Creutzfeld-Jakob disease and the gene is PRNP. In some embodiments, the disease comprises Familial Mediterranean Fever and the gene isMEFV. In some embodiments, the disease comprises Friedreich's ataxia and the gene is FAN. In some embodiments, the disease comprises Gaucher disease and the gene is GBA. In some embodiments, the disease comprises human papilloma virus (HPV) infection and the gene is HPV E7. In some embodiments, the disease comprises hemochromatosis and the gene is HFE, optionally comprising a C282Y mutation. In some embodiments, the disease comprises Hemophilia A and the gene is FVIII. In some embodiments, the disease is hereditary angioedema and the gene is SERPING1 or KLKB1. In some embodiments, the disease comprises histiocytosis and the gene is CDL. In some embodiments, the disease comprises immunodeficiency 17 and the gene is CD3D. In some embodiments, the disease comprises immunodeficiency 13 and the gene is CD4. In some embodiments, the disease comprises Common Variable Immunodeficiency and the gene is selected from CD19 and CD81. In some embodiments, the disease comprises Joubert syndrome and the gene is selected from INPP5E, TMEM216, AHI1, NPHP1, CEP290, TMEM67, RPGRIP1L, ARL13B, CC2D2A, OFD1, TMEM138, TCTN3, ZNF423, and AMRC9. In some embodiments, the disease comprises leukocyte adhesion deficiency and the gene is CD18. In some embodiments, the disease comprises Li-Fraumeni syndrome and the gene is TP53. In some embodiments, the disease comprises lymphoproliferative syndrome and the gene is CD27. In some embodiments, the disease comprises Lynch syndrome and the gene is selected from MSH2, MLH1, MSH6, PMS2, PMS1, TGFBR2, and MLH3. In some embodiments, the disease comprises mantle cell lymphoma and the gene is CD5. In some embodiments, the disease comprises Marfan syndrome and the gene is FBNJ. In some embodiments, the disease comprises mastocytosis and the gene is CD2. In some embodiments, the disease comprises methylmalonic acidemia and the gene is selected from MAA, MMAB, and MUT. In some embodiments, the disease is mycosis fungoides and the gene is CD7. In some embodiments, the disease is myotonic dystrophy and the gene is selected from CNBP and DMPK. In some embodiments, the disease comprises neurofibromatosis and the gene is selected from NF1, and NF2. In some embodiments, the disease comprises osteogenesis imperfecta and the gene is selected from COLIA1, COLIA2, and IFITM5. In some embodiments, the disease is non-small cell lung cancer and the gene is selected from KRAS, EGFR, ALK, METex14, BRAF V600E, ROS1, RET, and NTRK. In some embodiments, the disease comprises Peutz-Jeghers syndrome and the gene is STK11. In some embodiments, the disease comprises polycystic kidney disease and the gene is selected from PKD1 and PKD2. In some embodiments, the disease comprises Severe Combined Immune Deficiency and the gene is selected from IL7R, RAG1, and JAK3. In some embodiments, the disease comprises PRKAG2 cardiac syndrome and the gene is PRKAG2. In some embodiments, the disease comprises spinocerebellar ataxia and the gene is selected from ATXN1, ATXN2, ATXN3, PLEKHG4, SPTBN2, CACNAIA, ATXN7, ATXN8OS, ATXN10, TTBK2, PPP2R2B, KCNC3, PRKCG, ITPR1, TBP, KCND3, and FGF14. In some embodiments, the disease is thrombophilia due to antithrombin III deficiency and the gene is SERPINCL. In some embodiments the disease is spinal muscular atrophy and the gene is SMN1. In some embodiments, the disease comprises Usher Syndrome and the gene is selected from MYO7A, USH1C, CDH23, PCDH15, USH1G, USH2A, GPR98, DFNB31, and CLRN1. In some embodiments, the disease comprises von Willebrand disease and the gene is VWF. In some embodiments, the disease comprises Waardenburg syndrome and the gene is selected from PAX3, MITF, WS2B, WS2C, SNAI2, EDNRB, EDN3, and SOX10. In some embodiments, the disease comprises Wiskott-Aldrich Syndrome and the gene is WAS. In some embodiments, the disease comprises von Hippel-Lindau disease and the gene is VHL. In some embodiments, the disease comprises Wilson disease and the gene is ATP7B. In some embodiments, the disease comprises Zellweger syndrome and the gene is selected from PEX1, PEX2, PEX3, PEX5, PEX6, PEX10, PEX12, PEX13, PEX14, PEX16, PEX19, and PEX26. In some embodiments, the disease comprises infantile myofibromatosis and the gene is CD34. In some embodiments, the disease comprises platelet glycoprotein IV deficiency and the gene is CD36. In some embodiments, the disease comprises immunodeficiency with hyper-IgM type 3 and the gene is CD40. In some embodiments, the disease comprises hemolytic uremic syndrome and the gene is CD46. In some embodiments, the disease comprises complement hyperactivation, angiopathic thrombosis, or protein-losing enteropathy and the gene is CD55. In some embodiments, the disease comprises hemolytic anemia and the gene is CD59. In some embodiments, the disease comprises calcification of joints and arteries and the gene is CD73. In some embodiments, the disease comprises immunoglobulin alpha deficiency and the gene is CD79A. In some embodiments, the disease comprises C syndrome and the gene is CD96. In some embodiments, the disease comprises hairy cell leukemia and the gene is CD123. In some embodiments, the disease comprises histiocytic sarcoma and the gene is CD163. In some embodiments, the disease comprises autosomal dominant deafness and the gene is CD164. In some embodiments, the disease comprises immunodeficiency 25 and the gene is CD247. In some embodiments, the disease comprises methymalonic acidemia due to transcobalamin receptor defect and the gene is CD320.

### Cancer

In some embodiments, compositions, systems or methods described herein edit at least one gene associated with a cancer or the expression thereof. Non-limiting examples of cancers include: acute lymphoblastic leukemia; acute lymphoblastic lymphoma; acute lymphocytic leukemia; acute myelogenous leukemia; acute myeloid leukemia (adult/childhood); adrenocortical carcinoma; anal cancer; appendix cancer; astrocytoma; atypical teratoid/rhabdoid tumor; basal-cell carcinoma; bile duct cancer; bladder cancer; bone osteosarcoma; brain cancer; brain tumor; brainstem glioma; breast cancer; bronchial adenoma, carcinoid, or tumor; Burkitt lymphoma; carcinomacervical cancer; chronic lymphocytic leukemia; chronic myelogenous leukemia; chronic myeloid leukemia; colon cancer; colorectal cancer; emphysema; endometrial cancer; esophageal cancer; Ewing sarcoma; gallbladder cancer; gastric (stomach) cancer; gastrointestinal tumor; gliomahairy cell leukemia; head and neck cancer; liver cancer; Hodgkin's lymphoma; hypopharyngeal cancer; Kaposi Sarcoma; kidney cancer lip and oral cavity cancer; liposarcoma; lung cancer, non-small cell lung cancer; Waldenstrom; melanoma; mesotheliomamyelogenous leukemia; myeloid leukemia; myeloma; nasopharyngeal carcinoma; neuroblastoma; non-Hodgkin's lymphoma; ovarian cancer; pancreatic cancer; pineal cancer; pituitary tumor; prostate cancer; rectal cancer; renal cell carcinomaretinoblastoma; spinal cord tumor; squamous cell carcinoma; squamous neck cancer; T-cell lymphoma, cutaneous (Mycosis Fungoides and Sezary syndrome); testicular cancer; throat cancer; thyroid cancer; urethral cancer; uterine cancervaginal cancer; and Wilms Tumor. In some embodiments, the cancer is a solid cancer (i.e., a tumor). In some embodiments, the cancer is selected from a blood cell cancer, a leukemia, and a lymphoma. The cancer can be a leukemia, such as, by way of non-limiting example, acute myeloid (or myelogenous) leukemia (AML), chronic myeloid (or myelogenous) leukemia (CMIL), acute lymphocytic (or lymphoblastic) leukemia (ALL), and chronic lymphocytic leukemia (CLL). In some embodiments, the cancer is any one of colon cancer, rectal cancer, renal-cell carcinoma, liver cancer, bladder cancer, cancer of the kidney or ureter, lung cancer, non-small cell lung cancer, cancer of the small intestine, esophageal cancer, melanoma, bone cancer, pancreatic cancer, skin cancer, brain cancer (e.g., glioblastoma), cancer of the head or neck, melanoma, uterine cancer, ovarian cancer, breast cancer, testicular cancer, cervical cancer, stomach cancer, Hodgkin's Disease, non-Hodgkin's lymphoma, and thyroid cancer.

In some embodiments, compositions, systems or methods described herein edit at least one mutation in a target nucleic acid, wherein the at least one mutation is associated with cancer or causative of cancer. In some embodiments, the target nucleic acid comprises a gene associated with cancer, a gene whose overexpression is associated with cancer, a tumor suppressor gene, an oncogene, a checkpoint inhibitor gene, a gene associated with cellular growth, a gene associated with cellular metabolism, a gene associated with cell cycle, combinations thereof, or portions thereof. Non-limiting examples of genes comprising a mutation associated with cancer are ABL, ACE, AF4/HRX, AKT-2, ALK, ALK/NPM, AML1, AMLJ/MTG8, APC, ATM, AXIN2, AXL, BAP1, BARD1, BCL-2, BCL-3, BCL-6, BCR/ABL, BLM, BMPR1A, BRCA1, BRCA2, BRIP1, c-MYC, CASR, CCR5, CDC73, CDH1, CDK4, CDKN1B, CDKN1C, CDKN2A, CEBPA, CHEK2, CREBBP, CTNNA1, DBL, DEK/CAN, DICER1, DIS3L2, E2A/PBX1, EGFR, ENL/HRX EPCAM, ERG/TLS, ERBB, ERBB-2, ETS-1, EWSFLI-1, FH, FKRP, FLCN, FMS, FOS, FPS, GATA2, GCG, GLI, GPC3, GPGSP, GREM1, HER2/neu, HOX11, HOXB13, HRAS, HST, IL-3, INT-2, JAK1, JUN, KIT, KS3, K-SAM, LBC, LCK, LMO1, LMO2, L-MYC, LYL-1, LYT-10, LYT-10/Cα1, MAS, MAX, MDM-2, MEN1, MET, MITF, MLH1, MLL, MOS, MSH1, MSH2, MSH3, MSH6, MTG8/AML1, MUTYH, MYB, MYH11/CBFB, NBN, NEU, NF1, NF2, N-MYC, NTHL1, OST, PALB2, PAX-5, PBX1/E2A, PCDC1, PDGFRA, PHOX2B, PIM-1, PMS2, POLD1, POLE, POT1, PPARG, PRAD-1, PRKARIA, PTCH1, PTEN, RAD50, RAD51C, RAD51D, RAF, RAR/PML, RAS-H, RAS-K, RAS-N, RB1, RECQL4, REL/NRG, RET, RHOM1, RHOM2, ROS, RUNX1, SDHA, SDHAF, SDHAF2, SDHB, SDHC, SDHD, SET/CAN, SIS, SKI, SMAD4, SMARCA4, SMARCB1, SMARCE1, SRC, STK11, SUFU, TAL1, TAL2, TAN-1, TIAM1, TERC, TERT, TIMP3, TMEM127, TNF, TP53, TRAC, TSC1, TSC2, TRK, VHL, WRN, and WT1. Non-limiting examples of oncogenes are KRAS, NRAS, BRAF, MYC, CTNNB1, and EGFR. In some embodiments, the oncogene is a gene that encodes a cyclin dependent kinase (CDK). Non-limiting examples of CDKs are Cdk1, Cdk4, Cdk5, Cdk7, Cdk8, Cdk9, Cdk11 and CDK20. Non-limiting examples of tumor suppressor genes are TP53, RB1, and PTEN.

### Infections

In some embodiments, compositions, systems or methods described herein treats an infection in a subject. In some embodiments, the infections are caused by a pathogen (e.g., bacteria, viruses, fungi, and parasites). In some embodiments, compositions, systems or methods described herein modifies a target nucleic acid associated with the pathogen or parasite causing the infection. In some embodiments, the target nucleic acid may be in the pathogen or parasite itself or in a cell, tissue or organ of the subject that the pathogen or parasite infects. In some embodiments, the methods described herein include treating an infection caused by one or more bacterial pathogens. Non-limiting examples of bacterial pathogens include Acholeplasma laidlawii, Brucella abortus, Chlamydia psittaci, Chlamydia trachomatis, Cryptococcus neoformans, Escherichia coli, Legionella pneumophila, Lyme disease spirochetes, methicillin-resistant Staphylococcus aureus, Mycobacterium leprae, Mycobacterium tuberculosis, Mycoplasma arginini, Mycoplasma arthritidis, Mycoplasma genitalium, Mycoplasma hyorhinis, Mycoplasma orale, Mycoplasma pneumoniae, Mycoplasma salivarium, Neisseria gonorrhoeae, Neisseria meningitidis, Pneumococcus, Pseudomonas aeruginosa, sexually transmitted infection, Streptococcus agalactiae, Streptococcus pyogenes, and Treponema pallidum.

In some embodiments, compositions, systems or methods described herein treats an infection caused by one or more viral pathogens. Non-limiting examples of viral pathogens include adenovirus, blue tongue virus, chikungunya, coronavirus (e.g., SARS-CoV-2), cytomegalovirus, Dengue virus, Ebola, Epstein-Barr virus, feline leukemia virus, Hemophilus influenzae B, Hepatitis virus A, Hepatitis virus B, Hepatitis virus C, herpes simplex virus I, herpes simplex virus II, human papillomavirus (HPV) including HPV16 and HPV18, human serum parvo-like virus, human T-cell leukemia viruses, immunodeficiency virus (e.g., HIV), influenza virus, lymphocytic choriomeningitis virus, measles virus, mouse mammary tumor virus, mumps virus, murine leukemia virus, polio virus, rabies virus, Reovirus, respiratory syncytial virus (RSV), rubella virus, Sendai virus, simian virus 40, Sindbis virus, varicella-zoster virus, vesicular stomatitis virus, wart virus, West Nile virus, yellow fever virus, or any combination thereof.

In some embodiments, compositions, systems or methods described herein treats an infection caused by one or more parasites. Non-limiting examples of parasites include helminths, annelids, platyhelminthes, nematodes, and thorny-headed worms. In some embodiments, parasitic pathogens comprise, without limitation, Babesia bovis, Echinococcus granulosus, Eimeria tenella, Leishmania tropica, Mesocestoides corti, Onchocerca volvulus, Plasmodium falciparum, Plasmodium vivax, Schistosoma japonicum, Schistosoma mansoni, Schistosoma spp., Taenia hydatigena, Taenia ovis, Taenia saginata, Theileria parva, Toxoplasma gondii, Toxoplasma spp., Trichinella spiralis, Trichomonas vaginalis, Trypanosoma brucei, Trypanosoma cruzi, Trypanosoma rangeli, Trypanosoma rhodesiense, Balantidium coli, Entamoeba histolytica, Giardia spp., Isospora spp., Trichomonas spp., or any combination thereof.

### V. Systems

Disclosed herein are systems for detecting and/or editing target nucleic acid. In some embodiments, systems comprise components comprising one or more of: compositions described herein.

Disclosed herein is a system for modifying a target nucleic acid, wherein the system comprises: (a) an effector protein or a nucleic acid encoding the effector protein; (b) a guide nucleic acid or a nucleic acid encoding the guide nucleic acid, wherein the guide nucleic acid comprising a first region and a second region, wherein the first region binds to the effector protein and the second region hybridizes to a target sequence of the target strand; and (c) at least one of fusion partners as described herein. In some embodiments, the effector protein comprises an amino acid sequence that is at least 70%, at least 75%, at least 80%, at least 85%, at least 90%, at least 95%, at least 97%, at least 98%, at least 99% or 100% identical to any one of the sequences described in TABLE 1. In some embodiments, the at least one of the fusion partners comprise an amino acid sequence that is at least 70%, at least 75%, at least 80%, at least 85%, at least 90%, at least 95%, at least 97%, at least 98%, at least 99% or 100% identical to any one of the sequences described in TABLE 2.

### Systems for Targeted DNA Base Editing

Disclosed herein is a system for targeted DNA base editing, wherein the system comprises: (a) an effector protein or a nucleic acid encoding the effector protein; (b) a guide nucleic acid or a nucleic acid encoding the guide nucleic acid, wherein the guide nucleic acid comprising a first region and a second region, wherein the first region binds to the effector protein and the second region hybridizes to a target sequence of the target strand; and (c) a base editor or a nucleic acid encoding the base editor, wherein the base editor is optionally directly or indirectly linked to the effector protein.

### Systems for Inhibiting or Reducing Expression of a Target Nucleic Acid

Disclosed herein is a system for inhibiting or reducing expression of a target nucleic acid, wherein the system comprises: (a) an effector protein or a nucleic acid encoding the effector protein; (b) a guide nucleic acid or a nucleic acid encoding the guide nucleic acid, wherein the guide nucleic acid comprising a first region and a second region, wherein the first region binds to the effector protein and the second region hybridizes to a target sequence of the target strand of the target nucleic acid; and (c) a CRISPRi fusion partner or a nucleic acid encoding the CRISPRi fusion partner, wherein the CRISPRi fusion partner is optionally directly or indirectly linked to the effector protein.

### Systems for Inhibiting or Reducing Expression of a Target Nucleic Acid

Disclosed herein is a system for activating or increasing expression of a target nucleic acid, wherein the system comprises: (a) an effector protein or a nucleic acid encoding the effector protein; (b) a guide nucleic acid or a nucleic acid encoding the guide nucleic acid, wherein the guide nucleic acid comprising a first region and a second region, wherein the first region binds to the effector protein and the second region hybridizes to a target sequence of the target strand of the target nucleic acid; and (c) a CRISPRa fusion partner or a nucleic acid encoding the CRISPRa fusion partner, wherein the CRISPRa fusion partner is optionally directly or indirectly linked to the effector protein.

### Systems for Modular Organization

Disclosed herein is a system for modular organization, wherein the system comprises: (a) an effector protein or a nucleic acid encoding the effector protein; (b) a guide nucleic acid or a nucleic acid encoding the guide nucleic acid, wherein the guide nucleic acid comprising a first region and a second region, wherein the first region binds to the effector protein and the second region hybridizes to a target sequence of the target strand of the target nucleic acid; and (c) an RNA splicing factor or a nucleic acid encoding the RNA splicing factor, wherein the RNA splicing factor is optionally directly or indirectly linked to the effector protein.

### Systems for Recombination of a DNA

Disclosed herein is a system for recombination of a DNA, wherein the system comprises: (a) an effector protein or a nucleic acid encoding the effector protein; (b) a guide nucleic acid or a nucleic acid encoding the guide nucleic acid, wherein the guide nucleic acid comprising a first region and a second region, wherein the first region binds to the effector protein and the second region hybridizes to a target sequence of the target strand of the target nucleic acid; and (c) a recombinase or a nucleic acid encoding the recombinase, wherein the recombinase is optionally directly or indirectly linked to the effector protein.

Systems for targeted DNA alkylation

Disclosed herein is a system for targeted DNA alkylation, wherein the system comprises: (a) an effector protein or a nucleic acid encoding the effector protein; (b) a guide nucleic acid or a nucleic acid encoding the guide nucleic acid, wherein the guide nucleic acid comprising a first region and a second region, wherein the first region binds to the effector protein and the second region hybridizes to a target sequence of the target strand; (c) a DNA alkylating fusion partner or a nucleic acid encoding the DNA alkylating fusion partner, wherein the DNA alkylating fusion partner is optionally directly or indirectly linked to the effector protein. In some embodiments, the system further comprises a repair inhibitor fusion partner or a nucleic acid encoding the repair inhibitor fusion partner. In some embodiments, the system is used for targeted DNA alkylation.

Also, disclosed herein is a system for targeted DNA alkylation, wherein the system comprises: (a) at least one guide nucleic acid and an effector protein that binds to a first region of the guide nucleic acid, wherein a second region of the guide nucleic acid, upon contact with a double stranded DNA molecule comprising: (i) a target strand, and (ii) a non-target strand, hybridizes to the target strand of the double stranded DNA molecule; (b) a DNA alkylating fusion partner. In some embodiments, the system further comprises a repair inhibitor fusion partner.

Systems to selectively treat a genetic disorder associated with a genetic mutation

Disclosed herein is a system to selectively treat a genetic disorder associated with a genetic mutation, wherein the system comprises: (a) an effector protein or a nucleic acid encoding the effector protein; (b) a guide nucleic acid or a nucleic acid encoding the guide nucleic acid, wherein the guide nucleic acid comprising a first region and a second region, wherein the first region binds to the effector protein and the second region hybridizes to a target sequence of the target strand; and (c) a deaminase fusion partner or a nucleic acid encoding the deaminase fusion partner, wherein the methyl transferase fusion partner is optionally directly or indirectly linked to the effector protein. In some embodiments, the system further comprises a methyl transferase fusion partner or a nucleic acid encoding the methyl transferase fusion partner, wherein the methyl transferase fusion partner is optionally directly or indirectly linked to the effector protein. In some embodiments, the system further comprises a thymine DNA glycosylase inhibitor fusion partner or a nucleic acid encoding the thymine DNA glycosylase inhibitor fusion partner. In some embodiments, the system selectively treats a genetic disorder associated with a genetic mutation.

Also, herein is a system to selectively treat a genetic disorder associated with a genetic mutation, wherein the system comprises: (a) at least one guide nucleic acid and a effector protein that binds to a first region of the guide nucleic acid, wherein a second region of the guide nucleic acid, upon contact with a double stranded DNA molecule comprising: (i) a target strand, and (ii) a non-target strand that comprises the genetic mutation, hybridizes to the target strand of the double stranded DNA molecule; (b) a methyl transferase fusion partner; and (c) a deaminase fusion partner. In some embodiments, the system further comprises a thymine DNA glycosylase inhibitor fusion partner. In some embodiments, the system selectively treats a genetic disorder associated with a genetic mutation.

### Systems for Targeted Correction of a DNA Frameshift

Disclosed herein is a system for targeted correction of a DNA frameshift, wherein the system comprises: (a) an effector protein or a nucleic acid encoding the effector protein; (b) a guide nucleic acid or a nucleic acid encoding the guide nucleic acid, wherein the guide nucleic acid comprising a first region and a second region, wherein the first region binds to the effector protein and the second region hybridizes to a target sequence of the target strand; and (c) a terminal deoxynucleotidyl transferase (TdT) fusion partner or a nucleic acid encoding the TdT fusion partner, wherein the TdT fusion partner is optionally directly or indirectly linked to the effector protein. In some embodiments, the system further comprises a second guide RNA, wherein the second guide RNA recognizes a PAM sequence that is different from the PAM sequence recognized by the guide RNA. In some embodiments, the system is used for targeted correction of a DNA frameshift in a DNA molecule. In some embodiments, the system is used for targeted insertion of a sequence of nucleotides into the target sequence.

### Systems for Suppressing a Nonsense Codon

Disclosed herein is a system for suppressing a nonsense codon, wherein the system comprises: (a) an effector protein or a nucleic acid encoding the effector protein; (b) a guide nucleic acid or a nucleic acid encoding the guide nucleic acid, wherein the guide nucleic acid comprising a first region and a second region, wherein the first region binds to the effector protein and the second region hybridizes to a target sequence of the target strand; and (c) an RNA pseudouridylation fusion partner or a nucleic acid encoding the RNA pseudouridylation fusion partner, wherein the RNA pseudouridylation fusion partner is optionally directly or indirectly linked to the effector protein. In some embodiments, the target sequence is an mRNA transcript. In some embodiments, the system is used for suppressing a nonsense codon in an mRNA transcript.

### Systems for Targeted Nucleotide Modification

Disclosed herein is a system for targeted nucleotide modification, wherein the system comprises: (a) an effector protein or a nucleic acid encoding the effector protein; (b) a guide nucleic acid or a nucleic acid encoding the guide nucleic acid, wherein the guide nucleic acid comprising a first region and a second region, wherein the first region binds to the effector protein and the second region hybridizes to a target sequence of the target strand; and (c) a fusion partner or a nucleic acid encoding the fusion partner, wherein the fusion partner is optionally directly or indirectly linked to the effector protein, wherein the fusion partner comprises an N-alkylating fusion partner, an oxidizing fusion partner, a cytosine deaminating fusion partner, an apurinic or apyrimidinic site generating fusion partner, a ribonucleotide reductase fusion partner, or combinations thereof. In some embodiments, the system is used for performing a chemical modification of a nucleotide of the target sequence.

### VI. Genetically Modified Cells and Organisms

In some aspects, disclosed herein are cells or a populations of cells, wherein the cell comprises a fusion effector protein described herein. In some embodiments, the cell comprises a nucleic acid vector encoding a fusion effector protein described herein. In some embodiments, the cell is a prokaryotic cell. In some embodiments, the cell is a eukaryotic cell. In some embodiments, the cell is a mammalian cell.

Non-limiting examples of cells that may be engineered or modified with compositions and methods described herein include immune cells, such as CAR T-cells, T-cells, B-cells, NK cells, granulocytes, basophils, eosinophils, neutrophils, mast cells, monocytes, macrophages, dendritic cells, antigen-presenting cells (APC), or adaptive cells. Non-limiting examples of cells that may be engineered or modified with compositions and methods described herein include plant cells, such as parenchyma, sclerenchyma, collenchyma, xylem, phloem, germline (e.g., pollen). Cells from lycophytes, ferns, gymnosperms, angiosperms, bryophytes, charophytes, chloropytes, rhodophytes, or glaucophytes. Non-limiting examples of cells that may be engineered or modified with compositions and methods described herein include stem cells, such as human stem cells, animal stem cells, stem cells that are not derived from human embryonic stem cells, embryonic stem cells, mesenchymal stem cells, pluripotent stem cells, induced pluripotent stem cells (iPS), somatic stem cells, adult stem cells, hematopoietic stem cells, tissue-specific stem cells.

A cell may be in vitro. A cell may be in vivo. A cell may be ex vivo. A cell may be a cell in a cell culture. A cell may be one of a collection of cells. A cell may be a mammalian cell or derived from a mammalian cell. A cell may be a rodent cell or derived from a rodent cell. A cell may be a human cell or derived from a human cell. A cell may be a prokaryotic cell or derived from a prokaryotic cell. A cell may be a bacterial cell or may be derived from a bacterial cell. A cell may be an archaeal cell or derived from an archaeal cell. A cell may be a eukaryotic cell or derived from a eukaryotic cell. A cell may be a pluripotent stem cell. A cell may be a plant cell or derived from a plant cell. A cell may be an animal cell or derived from an animal cell. A cell may be an invertebrate cell or derived from an invertebrate cell. A cell may be a vertebrate cell or derived from a vertebrate cell. A cell may be a microbe cell or derived from a microbe cell. A cell may be a fungi cell or derived from a fungi cell. A cell may be from a specific organ or tissue.

Plant cells, such as Parenchyma, sclerenchyma, collenchyma, xylem, phloem, germline (e.g., pollen). Cells from lycophytes, ferns, gymnosperms, angiosperms, bryophytes, charophytes, chloropytes, rhodophytes, or glaucophytes. Non-limiting examples of cells that may be used with this disclosure also include stem cells, such as human stem cells, animal stem cells, stem cells that are not derived from human embryonic stem cells, embryonic stem cells, mesenchymal stem cells, pluripotent stem cells, induced pluripotent stem cells (iPS), somatic stem cells, adult stem cells, hematopoietic stem cells, tissue-specific stem cells.

### Agricultural Engineering

Compositions and methods of the disclosure may be used for agricultural engineering. For example, compositions and methods of the disclosure may be used to confer desired traits on a plant. A plant may be engineered for the desired physiological and agronomic characteristic using the present disclosure. In some embodiments, the target nucleic acid sequence comprises a nucleic acid sequence of a plant. In some embodiments, the target nucleic acid sequence comprises a genomic nucleic acid sequence of a plant cell. In some embodiments, the target nucleic acid sequence comprises a nucleic acid sequence of an organelle of a plant cell. In some embodiments, the target nucleic acid sequence comprises a nucleic acid sequence of a chloroplast of a plant cell.

The plant may be a dicotyledonous plant. The plant may be a monocotyledonous plant. Non-limiting examples of plants include plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses, wheat, maize, rice, millet, barley, tomato, apple, pear, strawberry, orange, acacia, carrot, potato, sugar beets, yam, lettuce, spinach, sunflower, rape seed, Arabidopsis, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini. A plant may include algae.

### Certain Target Nucleic Acids

Disclosed herein are compositions, systems and methods for modifying a target nucleic acid. In some embodiments, the target nucleic acid is a single stranded nucleic acid. Alternatively, or in combination, the target nucleic acid is a double stranded nucleic acid and is prepared into single stranded nucleic acids before or upon contacting the reagents. In some embodiments, the target nucleic acid is a double stranded nucleic acid. In some embodiments, the double stranded nucleic acid is DNA. The target nucleic acid may be an RNA. The target nucleic acids include but are not limited to mRNA, rRNA, tRNA, non-coding RNA, long non-coding RNA, and microRNA (miRNA). In some embodiments, the target nucleic acid is complementary DNA (cDNA) synthesized from a single-stranded RNA template in a reaction catalyzed by a reverse transcriptase. In some embodiments, the target nucleic acid is single-stranded RNA (ssRNA) or mRNA.

### Mutations

In some embodiments, target nucleic acids comprise a mutation. In some embodiments, a sequence comprising a mutation may be modified to a wildtype sequence with a composition, system or method described herein. In some embodiments, a sequence comprising a mutation may be detected with a composition, system or method described herein. WO2020142739, which is hereby incorporated by reference in its entirety, provides further compositions and methods for generating, amplifying, and detecting modified nucleic acids. The mutation may be a mutation of 1, 2, 3, 4, 5, 6, 7, 8, 9, 10, 11, 12, 13, 14, 15, 16, 17, 18, 19, 20 or more nucleotides. Non-limiting examples of mutations are insertion-deletion (indel), single nucleotide polymorphism (SNP), and frameshift mutations. In some embodiments, guide nucleic acids described herein hybridize to a region of the target nucleic acid comprising the mutation. The SNP may be located in a non-coding region or a coding region of a gene.

In some embodiments, target nucleic acids comprise a mutation, wherein the mutation is a SNP. The single nucleotide mutation or SNP may be associated with a phenotype of the sample or a phenotype of the organism from which the sample was taken. The SNP, in some embodiments, is associated with altered phenotype from wild type phenotype. The SNP may be a synonymous substitution or a nonsynonymous substitution. The nonsynonymous substitution may be a missense substitution, or a nonsense point mutation. The synonymous substitution may be a silent substitution. The mutation may be a deletion of one or more nucleotides. Often, the single nucleotide mutation, SNP, or deletion is associated with a disease such as cancer or a genetic disorder. The mutation, such as a single nucleotide mutation, a SNP, or a deletion, may be encoded in the sequence of a target nucleic acid from the germline of an organism or may be encoded in a target nucleic acid from a diseased cell, such as a cancer cell.

In some embodiments, target nucleic acids comprise a mutation, wherein the mutation is a deletion of 1, 2, 3, 4, 5, 6, 7, 8, 9, 10, 11, 12, 13, 14, 15, 16, 17, 18, 19, 20 or more nucleotides. The mutation may be a deletion of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. The mutation may be a deletion of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides.

In some embodiments, the target nucleic acid comprises a mutation associated with a disease. In some examples, a mutation associated with a disease refers to a mutation whose presence in a subject indicates that the subject is susceptible to, or suffers from, a disease, disorder, or pathological state. In some examples, a mutation associated with a disease refers to a mutation which causes the disease, contributes to the development of the disease, or indicates the existence of the disease. A mutation associated with a disease may also refer to any mutation which generates transcription or translation products at an abnormal level, or in an abnormal form, in cells affected by a disease relative to a control without the disease.

The mutation may cause the disease. The disease may comprise, at least in part, a cancer, an inherited disorder, an ophthalmological disorder, a neurological disorder, a blood disorder, a metabolic disorder, or a combination thereof. The disease may comprise, at least in part, a cancer. The disease may comprise, at least in part, an inherited disorder. The disease may comprise, at least in part, an ophthalmological disorder. The disease may comprise, at least in part, a neurological disorder. The disease may comprise, at least in part, a blood disorder. The disease may comprise, at least in part, a metabolic disorder. In some embodiments, the target nucleic acid comprises a mutation associated with a disease. The mutation may cause the disease. The disease may comprise an inherited disorder, an ophthalmological disorder, a neurological disorder, a blood disorder, a metabolic disorder, or a combination thereof. The disease may comprise, at least in part, a cancer. The disease may comprise, at least in part, an inherited disorder. The disease may comprise, at least in part, an ophthalmological disorder. The disease may comprise, at least in part, a neurological disorder. The disease may comprise, at least in part, a blood disorder. The disease may comprise, at least in part, a metabolic disorder.

In some embodiments, the neurological disorder comprises Duchenne muscular dystrophy, myotonic dystrophy Type 1, or cystic fibrosis. In some embodiments, the neurological disorder comprises Duchenne muscular dystrophy. In some embodiments, the neurological disorder comprises myotonic dystrophy Type 1. In some embodiments, the neurological disorder comprises cystic fibrosis. In some embodiments, the neurological disorder comprises a neurodegenerative disease.

The target nucleic acid, in some embodiments, comprises a portion of a gene comprising a mutation associated with cancer, a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes a cancer biomarker, such as a prostate cancer biomarker or non-small cell lung cancer. In some embodiments, the assay may be used to detect “hotspots” in target nucleic acids that may be predictive of lung cancer. In some embodiments, the target nucleic acid comprises a portion of a nucleic acid that is associated with a blood fever. In some embodiments, the target nucleic acid is a portion of a nucleic acid from a genomic locus, any DNA amplicon of, a reverse transcribed mRNA, or a cDNA from a locus of at least one of: ALK, APC, ATM, AXIN2, BAP1, BARD1, BLM, BMPR1A, BRCA1, BRCA2, BRIP1, CASR, CDC73, CDH1, CDK4, CDKN1B, CDKN1C, CDKN2A, CEBPA, CHEK2, CTNNA1, DICER1, DIS3L2, EGFR, EPCAM, FH, FLCN, GATA2, GPC3, GREM1, HOXB13, HRAS, MAX, MEN1, MET, MITF, MLH1, MSH2, MSH3, MSH6, MUTYH, NBN, NF1, NF2, NTHL1, PALB2, PDGFRA, PHOX2B, PMS2, POLD1, POLE, POT1, PRKAR1A, PTCH1, PTEN, RAD50, RAD51C, RAD51D, RB1, RECQL4, RET, RUNX1, SDHA, SDHAF2, SDHB, SDHC, SDHD, SMAD4, SMARCA4, SMARCB1, SMARCE1, STK11, SUFU, TERC, TERT, TMEM127, TP53, TSC1, TSC2, VHL, WRN, and WT1. Any region of the aforementioned gene loci may be probed for a mutation or deletion using the compositions and methods disclosed herein. For example, in the EGFR gene locus, the compositions and methods for detection disclosed herein may be used to detect a single nucleotide polymorphism or a deletion.

In some embodiments, the gene is PCSK9. In some embodiments, the gene is TRAC, B2M, PD1, or a combination thereof. In some embodiments, the contacting occurs in vitro. In some embodiments, the contacting occurs in vivo. In some embodiments, the contacting occurs ex vivo.

In some embodiments, the target nucleic acid comprises a portion of a nucleic acid from a genomic locus, any DNA amplicon of, a reverse transcribed mRNA, or a cDNA from a locus of at least one of: DNMT1, HPRT1, RPL32P3, CCR5, FANCF, GRIN2B, and EMX1. In some embodiments, the target nucleic acid comprises a portion of a nucleic acid from a genomic locus, any DNA amplicon of, a reverse transcribed mRNA, or a cDNA from DNMT1. In some embodiments, the target nucleic acid comprises a portion of a nucleic acid from a genomic locus, any DNA amplicon of, a reverse transcribed mRNA, or a cDNA from HPRT1. In some embodiments, the target nucleic acid comprises a portion of a nucleic acid from a genomic locus, any DNA amplicon of, a reverse transcribed mRNA, or a cDNA from RPL32P3. In some embodiments, the target nucleic acid comprises a portion of a nucleic acid from a genomic locus, any DNA amplicon of, a reverse transcribed mRNA, or a cDNA from CCR5. In some embodiments, the target nucleic acid comprises a portion of a nucleic acid from a genomic locus, any DNA amplicon of, a reverse transcribed mRNA, or a cDNA from FANCF. In some embodiments, the target nucleic acid comprises a portion of a nucleic acid from a genomic locus, any DNA amplicon of, a reverse transcribed mRNA, or a cDNA from GRIN2B. In some embodiments, the target nucleic acid comprises a portion of a nucleic acid from a genomic locus, any DNA amplicon of, a reverse transcribed mRNA, or a cDNA from EMX1. DNMT1, HPRT1, RPL32P3, CCR5, FANCF, GRIN2B, or EMX1 has been described in more detail in Kim et al., “Enhancement of target specificity of CRISPR-Cas12a by using a chimeric DNA-RNA guide”, Nucleic Acids Res. 2020 Sep. 4; 48(15):8601-8616, which is hereby incorporated by reference in its entirety.

In some embodiments, the target nucleic acid comprises a portion of a nucleic acid from a genomic locus, any DNA amplicon of, a reverse transcribed mRNA, or a cDNA from a locus of at least one of: AAVS1, ALKBH5, CLTA, and CDK11. In some embodiments, the target nucleic acid comprises a portion of a nucleic acid from a genomic locus, any DNA amplicon of, a reverse transcribed mRNA, or a cDNA from AAVS1. In some embodiments, the target nucleic acid comprises a portion of a nucleic acid from a genomic locus, any DNA amplicon of, a reverse transcribed mRNA, or a cDNA from ALKBH5. In some embodiments, the target nucleic acid comprises a portion of a nucleic acid from a genomic locus, any DNA amplicon of, a reverse transcribed mRNA, or a cDNA from CLTA. In some embodiments, the target nucleic acid comprises a portion of a nucleic acid from a genomic locus, any DNA amplicon of, a reverse transcribed mRNA, or a cDNA from CDK11. AAVS1, ALKBH5, CLTA, or CDK11 has been described in more detail in Wang et al., “Specificity profiling of CRISPR system reveals greatly enhanced off-target gene editing”, Scientific Reports volume 10, Article number: 2269 (2020), which is hereby incorporated by reference in its entirety.

In some embodiments, the target nucleic acid comprises a portion of a nucleic acid from a genomic locus, any DNA amplicon of, a reverse transcribed mRNA, or a cDNA from a locus of at least one of: CTNNB1, AXIN1, LRP6, TBK1, BAP1, TLE3, PPM1A, BCL2L2, SUFU, RICTOR, VPS35, TOP1, SIRT1, and PTEN. In some embodiments, the target nucleic acid comprises a portion of a nucleic acid from a genomic locus, any DNA amplicon of, a reverse transcribed mRNA, or a cDNA from CTNNB1. In some embodiments, the target nucleic acid comprises a portion of a nucleic acid from a genomic locus, any DNA amplicon of, a reverse transcribed mRNA, or a cDNA from AXIN1. In some embodiments, the target nucleic acid comprises a portion of a nucleic acid from a genomic locus, any DNA amplicon of, a reverse transcribed mRNA, or a cDNA from LRP6. In some embodiments, the target nucleic acid comprises a portion of a nucleic acid from a genomic locus, any DNA amplicon of, a reverse transcribed mRNA, or a cDNA from TBK1. In some embodiments, the target nucleic acid comprises a portion of a nucleic acid from a genomic locus, any DNA amplicon of, a reverse transcribed mRNA, or a cDNA from BAP1. In some embodiments, the target nucleic acid comprises a portion of a nucleic acid from a genomic locus, any DNA amplicon of, a reverse transcribed mRNA, or a cDNA from TLE3. In some embodiments, the target nucleic acid comprises a portion of a nucleic acid from a genomic locus, any DNA amplicon of, a reverse transcribed mRNA, or a cDNA from PPM1A. In some embodiments, the target nucleic acid comprises a portion of a nucleic acid from a genomic locus, any DNA amplicon of, a reverse transcribed mRNA, or a cDNA from BCL2L2. In some embodiments, the target nucleic acid comprises a portion of a nucleic acid from a genomic locus, any DNA amplicon of, a reverse transcribed mRNA, or a cDNA from SUFU. In some embodiments, the target nucleic acid comprises a portion of a nucleic acid from a genomic locus, any DNA amplicon of, a reverse transcribed mRNA, or a cDNA from RICTOR. In some embodiments, the target nucleic acid comprises a portion of a nucleic acid from a genomic locus, any DNA amplicon of, a reverse transcribed mRNA, or a cDNA from VPS35. In some embodiments, the target nucleic acid comprises a portion of a nucleic acid from a genomic locus, any DNA amplicon of, a reverse transcribed mRNA, or a cDNA from TOP1. In some embodiments, the target nucleic acid comprises a portion of a nucleic acid from a genomic locus, any DNA amplicon of, a reverse transcribed mRNA, or a cDNA from SIRT1. In some embodiments, the target nucleic acid comprises a portion of a nucleic acid from a genomic locus, any DNA amplicon of, a reverse transcribed mRNA, or a cDNA from PTEN. CTNNB1, AXIN1, LRP6, TBK1, BAP1, TLE3, PPM1A, BCL2L2, SUFU, RICTOR, VPS35, TOP1, SIRT1, or PTEN has been described in more detail in Tuladhar et al., “CRISPR-Cas9-based mutagenesis frequently provokes on-target mRNA misregulation”, Nature Communications volume 10, Article number: 4056 (2019), which is hereby incorporated by reference in its entirety.

In some embodiments, the target nucleic acid comprises a portion of a nucleic acid from a genomic locus, any DNA amplicon of, a reverse transcribed mRNA, or a cDNA from a locus of at least one of: MMD and PAQR8. In some embodiments, the target nucleic acid comprises a portion of a nucleic acid from a genomic locus, any DNA amplicon of, a reverse transcribed mRNA, or a cDNA from MMD. In some embodiments, the target nucleic acid comprises a portion of a nucleic acid from a genomic locus, any DNA amplicon of, a reverse transcribed mRNA, or a cDNA from PAQR8. MMD or PAQR8 has been described in more detail in Dong et al., “Genome-Wide Off-Target Analysis in CRISPR-Cas9 Modified Mice and Their Offspring”, G3, Volume 9, Issue 11, 1 Nov. 2019, Pages 3645-3651, which is hereby incorporated by reference in its entirety.

In some embodiments, the target nucleic acid comprises a portion of a nucleic acid from a genomic locus, any DNA amplicon of, a reverse transcribed mRNA, or a cDNA from a locus of at least one of: H2AX, POU5F1, and OCT4. In some embodiments, the target nucleic acid comprises a portion of a nucleic acid from a genomic locus, any DNA amplicon of, a reverse transcribed mRNA, or a cDNA from H2AX. In some embodiments, the target nucleic acid comprises a portion of a nucleic acid from a genomic locus, any DNA amplicon of, a reverse transcribed mRNA, or a cDNA from POU5F1. In some embodiments, the target nucleic acid comprises a portion of a nucleic acid from a genomic locus, any DNA amplicon of, a reverse transcribed mRNA, or a cDNA from OCT4.

In some embodiments, the target nucleic acid comprises a portion of a nucleic acid from a genomic locus, any DNA amplicon of, a reverse transcribed mRNA, or a cDNA from a locus of at least one of: SYS1, ARFRP1, and TSPAN14. In some embodiments, the target nucleic acid comprises a portion of a nucleic acid from a genomic locus, any DNA amplicon of, a reverse transcribed mRNA, or a cDNA from SYS1. In some embodiments, the target nucleic acid comprises a portion of a nucleic acid from a genomic locus, any DNA amplicon of, a reverse transcribed mRNA, or a cDNA from ARFRP1. In some embodiments, the target nucleic acid comprises a portion of a nucleic acid from a genomic locus, any DNA amplicon of, a reverse transcribed mRNA, or a cDNA from TSPAN14. SYS1, ARFRP1, or TSPAN14 has been described in more detail in Winter et al., “Genome-wide CRISPR screen reveals novel host factors required for Staphylococcus aureus α-hemolysin-mediated toxicity”, Scientific Reports volume 6, Article number: 24242 (2016), which is hereby incorporated by reference in its entirety.

In some embodiments, the target nucleic acid comprises a portion of a nucleic acid from a genomic locus, any DNA amplicon of, a reverse transcribed mRNA, or a cDNA from a locus of at least one of: EMC2, EMC3, SEL1L, DERL2, UBE2G2, UBE2J1, and HRD1. In some embodiments, the target nucleic acid comprises a portion of a nucleic acid from a genomic locus, any DNA amplicon of, a reverse transcribed mRNA, or a cDNA from EMC2. In some embodiments, the target nucleic acid comprises a portion of a nucleic acid from a genomic locus, any DNA amplicon of, a reverse transcribed mRNA, or a cDNA from EMC3. In some embodiments, the target nucleic acid comprises a portion of a nucleic acid from a genomic locus, any DNA amplicon of, a reverse transcribed mRNA, or a cDNA from SEL1L. In some embodiments, the target nucleic acid comprises a portion of a nucleic acid from a genomic locus, any DNA amplicon of, a reverse transcribed mRNA, or a cDNA from DERL2. In some embodiments, the target nucleic acid comprises a portion of a nucleic acid from a genomic locus, any DNA amplicon of, a reverse transcribed mRNA, or a cDNA from UBE2G2. In some embodiments, the target nucleic acid comprises a portion of a nucleic acid from a genomic locus, any DNA amplicon of, a reverse transcribed mRNA, or a cDNA from UBE2J1. In some embodiments, the target nucleic acid comprises a portion of a nucleic acid from a genomic locus, any DNA amplicon of, a reverse transcribed mRNA, or a cDNA from HRD1. EMC2, EMC3, SEL1L, DERL2, UBE2G2, UBE2J1, or HRD1 has been described in more detail in Ma et al., “A CRISPR-Based Screen Identifies Genes Essential for West-Nile-Virus-Induced Cell Death”, Cell Rep. 2015 Jul. 28; 12(4):673-83, which is hereby incorporated by reference in its entirety.

In some embodiments, the genetic disorder is hemophilia, sickle cell anemia, β-thalassemia, Duchenne muscular dystrophy, severe combined immunodeficiency, Huntington's disease, alpha-1 antitrypsin deficiency, or cystic fibrosis. The target nucleic acid, in some embodiments, is from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some embodiments, the target nucleic acid is encoded by a gene selected from: AAVS1, ABCA4, ABCB11, ABCC8, ABCD1, ABCG5, ABCG8, ACAD9, ACADM, ACADVL, ACAT1, ACOX1, ACSF3, ADA, ADAMTS2, ADGRG1, AGA, AGL, AGPS, AGXT, AHI, AIRE, ALDH3A2, ALDOB, ALG6, ALK, ALKBH5, ALMS1, ALPL, AMRC9, AMT, ANAPC10, ANAPC11, ANGPTL3, ANGPTL4, APC, Apo(α), APOCIII, APOEε4, APOL1, APP, AQP2, AR, ARFRP1, ARG1, ARH, ARL13B, ARL6, ARSA, ARSB, ASL, ASNS, ASPA, ASS1, ATM, ATP6V1B1, ATP7A, ATP7B, ATRX, ATXN1, ATXN10, ATXN2, ATXN3, ATXN7, ATXN8OS, AXIN1, AXIN2, B2M, BACE-1, BAK1, BAP1, BARD1, BAX2, BBS1, BBS10, BBS12, BBS2, BCKDHA, BCKDHB, BCL2L2, BCS1L, BEST1, Betaglobin gene, BLM, BMPR1A, BRAF, BRAFV600E, BRCA1, BRCA2, BRIP1, BSND, C9orf72, CA4, CACNA1A, CAH, CAPN3, CASR, CBS, CCNB1 CC2D2A, CCR5, CD1, CD2, CD3, CD3D, CD3Z, CD4, CD5, CD6, CD7, CD8A, CD8B, CD9, CD14, CD18, CD19, CD21, CD22, CD23, CD27, CD28, CD30, CD33, CD34, CD36, CD38, CD40, CD40L, CD44, CD46, CD47, CD48, CD52, CD55, CD57, CD58, CD59, CD68, CD69, CD72, CD73, CD74, CD79A, CD80, CD81, CD83, CD84, CD86, CD90, CD93, CD96, CD99, CD100, CD123, CD160, CD163, CD164, CD164L2, CD166, CD200, CD204, CD207, CD209, CD226, CD244, CD247, CD274, CD276, CD300, CD320, CDC73, CDH1, CDH23, CDK11, CDK4, CDKN1A, CDKN1B, CDKN1C, CDKN2A, CDKN2B, CEBPA, CELA3B, CEP290, CERKL, CFB, CFTR, CHCHD10, CHEK2, CHM, CHRNE, CIDEB, CIITA, CLN3, CLN5, CLN6, CLN8, CLRN1, CLTA, CMT1A, CNBP, CNGB1, CNGB3, COL1A1, COL1A2, COL27A1, COL4A3, COL4A4, COL4A5, COL7A1, CPS1, CPT1A, CPT2, CRB1, CREBBP, CRX CRYAA, CTNNA1, CTNNB1, CTNND2, CTNS, CTSK, CXCL12, CYBA, CYBB, CYP11B1, CYP11B2, CYP17A1, CYP19A1, CYP21A2, CYP27A1, DBT, DCC, DCLRE1C, DERL2, DFNA36, DFNB31, DGAT2, DHCR7, DHDDS, DICER1, DIS3L2, DLD, DMD, DMPK, DNAH5, DNAI1, DNAI2, DNM2, DNMT1, DPC4, DYSF, EDA, EDN3, EDNRB, EGFR, EIF2B5, EMC2, EMC3, EMD, EMX1, EN1, EPCAM, ERCC6, ERCC8, ESCO2, ETFA, ETFDH, ETHE1, EVC, EVC2, EYS, F5, F9, FXI, FAH, FAM161A, FANCA, FANCB, FANCC, FANCD1, FANCD2, FANCE, FANCF, FANCG, FANCI, FANCJ, FANCL, FANCM, FANCN, FANCP, FANCS, FBN1, FGF14, FGFR2, FGFR3, FGA, FGB, FGG, FH, FHL1, FIX FKRP, FKTN, FLCN, FMR1, FOXP3, FSCN2, FSHD1, FUS, FUT8, FVIII, FXII, FXN, G6PC, GAA, GALC, GALK1, GALT, GAMT, GATA2, GATA-4, GBA, GBE1, GCDH, GCGR, GDNF, GFAP, GFM1, GHR, GJB1, GJB2, GLA, GLB1, GLDC, GLE1, GNE, GNPTAB, GNPTG, GNS, GPAM, GPC3, GPR98, GREM1, GRHPR, GRIN2B, H2AFX H2AX HADHA, HAX1, HBA1, HBA2, HBB, HBV cccDNA, HER2, HEXA, HEXB, HFE, HGSNAT, HLCS, HMGCL, HAO1, HOGA1, HOXB13, HPRPF3, HPRT1, HPS1, HPS3, HRAS, HRD1, HSD3B2, HSDI7B4, HSDI7B13, HTT, HUS1, HYAL1, HYLS1, IDS, IDUA, IFITM5, IKBKAP, IL2RG, IL7R, IMPDH1, INPP5E, IRF4, ITGB2, ITPR1, IVD, JAG1, JAK1, JAK3, KCNC3, KCND3, KCNJ11, KLKB1, KLHL7, KRAS, LAMA2, LAMA3, LAMB3, LAMC2, LCA5, LDHA, LDLR, LDLRAP1, LHX3, LIFR, LIPA, LMNA, LOR, LOXHD1, LPA, LPL, LRAT, LRP6, LRPPRC, LRRK2, MADR2, MAN2B1, MAPT, MARC1, MAX, MCM6, MCOLN1, MECP2, MED17, MEFV, MEN1, MERTK, MESP2, MET, METex14, MFN2, MFSD8, MIA3, MITF, MKL2, MKS1, MLC1, MLH1, MLH3, MMAA, MMAB, MMACHC, MMADHC, MMD, MPI, MPL, MPV17, MSH2, MSH3, MSH6, MTHFD1L, MTHFR, MTM1, MTRR, MTTP, MUT, MUTYH, MYC, MYH7, MYO7A, NAGLU, NAGS, NBN, NDRG1, NDUFAF5, NDUFS6, NEB, NF1, NF2, NKX2-5, NOG, NOTCH1, NOTCH2, NPC1, NPC2, NPHP1, NPHS1, NPHS2, NRAS, NR2E3, NTHL1, NTRK, NTRK1, OAT, OCT4, OFD1, OPA3, OTC, PAH, PALB2, PAQR8, PAX3, PC, PCCA, PCCB, PCDH15, PCSK9, PD1, PDCD1, PDE6B, PDGFRA, PDHA1, PDHB, PEX1, PEX10, PEX12, PEX13, PEX14, PEX16, PEX19, PEX2, PEX26, PEX3, PEX5, PEX6, PEX7, PFKM, PHGDH, PHOX2B, PKD1, PKD2, PKHD1, PKK, PLEKHG4, PMM2, PMP22, PMS1, PMS2, PNPLA3, POLD1, POLE, POMGNT1, POT1, POU5F1, PPM1A, PPP2R2B, PPT1, PRCD, PRKAG2, PRKARIA, PRKCG, PRNP, PROM1, PROP1, PRPF31, PRPF8, PRPH2, PRPS1, PSAP, PSD3, PSD95, PSEN1, PSEN2, PSRC1, PTCH1, PTEN, PTS, PUS1, PYGM, RAB23, RAD50, RAD51C, RAD51D, RAG1, RAG2, RAPSN, RARS2, RB1, RDH12, RECQL4, RET, RHO, RICTOR, RMRP, ROS1, RP1, RP2, RPE65, RPGR, RPGRIP1L, RPL32P3, RS1, RTCA, RTEL1, RUNX1, SACS, SAMHDI, SCNIA, SCN2A, SDHA, SDHAF2, SDHB, SDHC, SDHD, SEL1 L, SEPSECS, SERPINA1, SERPINC1, SERPING1, SGCA, SGCB, SGCG, SGSH, SIRT1, SLC12A3, SLC12A6, SLC17A5, SLC22A5, SLC25A13, SLC25A15, SLC26A2, SLC26A4, SLC35A3, SLC35B4, SLC37A4, SLC39A4, SLC4A11, SLC6A8, SLC7A7, SMAD3, SMAD4, SMARCA4, SMARCAL1, SMARCB1, SMARCE1, SMN1, SMPD1, SNAI2, SNCA, SNRNP200, SOD1, SOX10, SPARA7, SPTBN2, STAR, STAT3, STK11, SUFU, SUMF1, SYNE1, SYNE2, SYS1, TARDBP, TAT, TBK1, TBP, TCIRG1, TCTN3, TECPR2, TERC, TERT, TFR2, TGFBR2, TGM1, TH, TLE3, TMEM127, TMEM138, TMEM216, TMEM43, TMEM67, TMPRSS6, TOP1, TOPORS, TP53, TPP1, TRAC, TRMU, TSC1, TSC2, TSFM, TSPAN14, TTBK2, TTC8, TTPA, TTR, TULP1, TYMP, UBE2G2, UBE2J1, UBE3A, USH1C, USH1G, USH2A, VEGF, VHL, VPS13A, VPS13B, VPS35, VPS45, VRK1, VSX2, VWF, WAS, WDR19, WDR48, WNT10A, WRN, WS2B, WS2C, WT1, XPA, XPC, XPF, XRCC3, YAP1, ZAC1, ZEB1, ZFYVE26, and ZNF423.

### EXAMPLES

The following examples are included for illustrative purposes only and are not intended to limit the scope of the disclosure. It will be understood by those of skill in the art that numerous and various modifications can be made to yield essentially similar results without departing from the spirit of the present disclosure.

### Example 1: Base Editing with CasΦ-Deaminase Fusions

Multiple nucleic acid vectors encoding fusion proteins were constructed for base editing. These fusion proteins comprised a catalytically inactive variant of a CRISPR Cas enzyme, also referred to as “dead CasΦ12” (SEQ ID NO: 7) fused to either ABE8e (SEQ ID NO: 400) or ABE8.20m (SEQ ID NO: 401) via an XTEN10 linker. The XTEN10 linker has a sequence of GSPAGSPTST (SEQ ID NO: 513), which is contained within the larger linker of GSGSPAGSPTSTRSGGGSGTS (SEQ ID NO: 517). These vectors encoded an amino acid sequence containing a nuclear localization signal (MPKKKRKVGIHGVPAA; SEQ ID NO: 603) fused to the dead CasΦ.12, but did not encode a uracil glycosylase inhibitor (UGI). Unfused dead CasΦ.12 served as a negative control. The amino acid sequences of these fusion proteins are provided in TABLE 4. These fusion proteins and twenty different guide RNAs per fusion protein were tested for their capability to edit multiple target sequences in eukaryotic mammalian (HEK293T) cells. Target sequences included sequences located in the genes, B2M, TRAC, FUT8, and PDCD1. Guide RNA sequences that provide base editing are provided in TABLE 5. Cells were transfected with the nucleic acid vectors and guide RNAs. After sufficient incubation, DNA was extracted from the transfected cells. Target sequences were PCR amplified and sequenced by NGS and MiSeq. The presence of base modifications was analyzed from sequencing data. A-to-G editing was observed 3′ of the target site, surrounding position 5. For reference, the last base in the PAM is position −1 and the first base after the PAM is position 1. In this case, for CasΦ.12, the PAM is NTTN from positions −4 to −1.

Results were recorded as a change in % base call relative to the negative control. Five of twenty gRNAs demonstrated 1.9% adenine to guanine editing in the spacer of the non-template (coding) strand. Editing window centered around positions 5-9. No editing was observed in the 10 bases immediately preceding and following the 5′ and 3′ ends of the spacer, respectively. 2.8% editing was achieved in follow-up experiments with additional gRNAs.

TABLE 4
CasΦ.12 Base Editors

| Fusion | |
|---|---|
| Name | Amino Acid Sequence |
| dCasΦ.12 | MIKPTVSQFLTPGFKLIRNHSRTAGLKLKNEGEEACKKFVRENEIPKDEC |
| (D369A)- | PNFQGGPAIANIIAKSREFTEWEIYQSSLAIQEVIFTLPKDKLPEPILKEEW |
| XTEN10- | RAQWLSEHGLDTVPYKEAAGLNLIIKNAVNTYKGVQVKVDNKNKNNL |
| ABE8e | AKINRKNEIAKLNGEQEISFEEIKAFDDKGYLLQKPSPNKSIYCYQSVSP |
| | KPFITSKYHNVNLPEEYIGYYRKSNEPIVSPYQFDRLRIPIGEPGYVPKW |
| | QYTFLSKKENKRRKLSKRIKNVSPILGIICIKKDWCVFDMRGLLRTNHW |
| | KKYHKPTDSINDLFDYFTGDPVIDTKANVVRFRYKMENGIVNYKPVRE |
| | KKGKELLENICDQNGSCKLATVAVGQNNPVAIGLFELKKVNGELTKTLI |
| | SRHPTPIDFCNKITAYRERYDKLESSIKLDAIKQLTSEQKIEVDNYNNNFT |
| | PQNTKQIVCSKLNINPNDLPWDKMISGTHFISEKAQVSNKSEIYFTSTDK |
| | GKTKDVMKSDYKWFQDYKPKLSKEVRDALSDIEWRLRRESLEFNKLS |
| | KSREQDARQLANWISSMCDVIGIENLVKKNNFFGGSGKREPGWDNFYK |
| | PKKENRWWINAIHKALTELSQNKGKRVILLPAMRTSITCPKCKYCDSKN |
| | RNGEKFNCLKCGIELNADIDVATENLATVAITAQSMPKPTCERSGDAKK |
| | PVRARKAKAPEFHDKLAPSYTVVLREAVKRPAATKKAGQAKKKKGSG |
| | SPAGSPTSTRSGGGSGTSSEVEFSHEYWMRHALTLAKRARDEREVPVG |
| | AVLVLNNRVIGEGWNRAIGLHDPTAHAEIMALRQGGLVMQNYRLIDAT |
| | LYVTFEPCVMCAGAMIHSRIGRVVFGVRNSKRGAAGSLMNVLNYPGM |
| | NHRVEITEGILADECAALLCDFYRMPRQVFNAQKKAQSSIN (SEQ ID |
| | NO: 530) |
| dCasΦ.12 | MIKPTVSQFLTPGFKLIRNHSRTAGLKLKNEGEEACKKFVRENEIPKDEC |
| (D369A)- | PNFQGGPAIANIIAKSREFTEWEIYQSSLAIQEVIFTLPKDKLPEPILKEEW |
| XTEN10- | RAQWLSEHGLDTVPYKEAAGLNLIIKNAVNTYKGVQVKVDNKNKNNL |
| ABE8.20m | AKINRKNEIAKLNGEQEISFEEIKAFDDKGYLLQKPSPNKSIYCYQSVSP |
| | KPFITSKYHNVNLPEEYIGYYRKSNEPIVSPYQFDRLRIPIGEPGYVPKW |
| | QYTFLSKKENKRRKLSKRIKNVSPILGIICIKKDWCVFDMRGLLRTNHW |
| | KKYHKPTDSINDLFDYFTGDPVIDTKANVVRFRYKMENGIVNYKPVRE |
| | KKGKELLENICDQNGSCKLATVAVGQNNPVAIGLFELKKVNGELTKTLI |
| | SRHPTPIDFCNKITAYRERYDKLESSIKLDAIKQLTSEQKIEVDNYNNNFT |
| | PQNTKQIVCSKLNINPNDLPWDKMISGTHFISEKAQVSNKSEIYFTSTDK |
| | GKTKDVMKSDYKWFQDYKPKLSKEVRDALSDIEWRLRRESLEFNKLS |
| | KSREQDARQLANWISSMCDVIGIENLVKKNNFFGGSGKREPGWDNFYK |
| | PKKENRWWINAIHKALTELSQNKGKRVILLPAMRTSITCPKCKYCDSKN |
| | RNGEKFNCLKCGIELNADIDVATENLATVAITAQSMPKPTCERSGDAKK |
| | PVRARKAKAPEFHDKLAPSYTVVLREAVKRPAATKKAGQAKKKKGSG |
| | SPAGSPTSTRSGGGSGTSSEVEFSHEYWMRHALTLAKRARDEREVPVG |
| | AVLVLNNRVIGEGWNRAIGLHDPTAHAEIMALRQGGLVMQNYRLYDA |
| | TLYSTFEPCVMCAGAMIHSRIGRVVFGVRNAKTGAAGSLMDVLHHPG |
| | MNHRVEITEGILADECAALLCRFFRMPRRVFNAQKKAQSSTD (SEQ ID |
| | NO: 531) |

TABLE 5
CasΦ.12 Base Editor Guide RNAs

| Guide | Nucleotide Sequence | Nucleotide Sequence |
|---|---|---|
| RNA ID | (DNA Sequence) | (RNA Sequence) |
| PL5122 | CTTTCAAGACTAATAGATTGCTC | CUUUCAAGACUAAUAGAUUGCUC |
| B2M 1_1 | CTTACGAGGAGACGGGCCGAGAT | CUUACGAGGAGACGGGCCGAGAU |
| | GTCTCGCTCC (SEQ ID NO: 532) | GUCUCGCUCC (SEQ ID NO: 773) |
| PL5128 | CTTTCAAGACTAATAGATTGCTC | CUUUCAAGACUAAUAGAUUGCUC |
| TRAC- | CTTACGAGGAGACGCATGTGCAA | CUUACGAGGAGACGCAUGUGCAA |
| target30 | ACGCCTTCAA (SEQ ID NO: 533) | ACGCCUUCAA (SEQ ID NO: 774) |
| PL5131 | CTTTCAAGACTAATAGATTGCTC | CUUUCAAGACUAAUAGAUUGCUC |
| TRAC- | CTTACGAGGAGACTCCCACAGAT | CUUACGAGGAGACUCCCACAGAU |
| target2 | ATCCAGAACC (SEQ ID NO: 534) | AUCCAGAACC (SEQ ID NO: 775) |
| PL5137 | CTTTCAAGACTAATAGATTGCTC | CUUUCAAGACUAAUAGAUUGCUC |
| B2M- | CTTACGAGGAGACATATAAGTGG | CUUACGAGGAGACAUAUAAGUG |
| target2 | AGGCGTCGCG (SEQ ID NO: 535) | GAGGCGUCGCG (SEQ ID NO: 776) |
| PL5138 | CTTTCAAGACTAATAGATTGCTC | CUUUCAAGACUAAUAGAUUGCUC |
| B2M- | CTTACGAGGAGACTGGCCTGGAG | CUUACGAGGAGACUGGCCUGGAG |
| target8 | GCTATCCAGC (SEQ ID NO: 536) | GCUAUCCAGC (SEQ ID NO: 777) |
| PL5123 | CTTTCAAGACTAATAGATTGCTC | CUUUCAAGACUAAUAGAUUGCUC |
| FUT8_T2 | CTTACGAGGAGACGCCTTAACAA | CUUACGAGGAGACGCCUUAACAA |
| | GCTGCTCTTC (SEQ ID NO: 537) | GCUGCUCUUC (SEQ ID NO: 778) |
| PL5126 | CTTTCAAGACTAATAGATTGCTC | CUUUCAAGACUAAUAGAUUGCUC |
| TRAC- | CTTACGAGGAGACGCACATGCAA | CUUACGAGGAGACGCACAUGCAA |
| target29 | AGTCAGATTT (SEQ ID NO: 538) | AGUCAGAUUU (SEQ ID NO: 779) |
| PL5127 | CTTTCAAGACTAATAGATTGCTC | CUUUCAAGACUAAUAGAUUGCUC |
| TRAC- | CTTACGAGGAGACGCACATGCAA | CUUACGAGGAGACGCACAUGCAA |
| target22 | AGTCAGATTT (SEQ ID NO: 538) | AGUCAGAUUU (SEQ ID NO: 779) |
| PL5139 | CTTTCAAGACTAATAGATTGCTC | CUUUCAAGACUAAUAGAUUGCUC |
| PDCD1- | CTTACGAGGAGACACACATGCCC | CUUACGAGGAGACACACAUGCCC |
| target87 | AGGCAGCACC (SEQ ID NO: 540) | AGGCAGCACC (SEQ ID NO: 781) |
| PL5140 | CTTTCAAGACTAATAGATTGCTC | CUUUCAAGACUAAUAGAUUGCUC |
| PDCD1- | CTTACGAGGAGACGAGCAGCCA | CUUACGAGGAGACGAGCAGCCAA |
| target75 | AGGTGCCCCTG (SEQ ID NO: 541) | GGUGCCCCUG (SEQ ID NO: 782) |

### Example 2: Activation of Gene Expression with CasΦ Fusion (CRISPRa)

An EGFP reporter was generated with a sequence that is known to be recognized by CasΦ.12-gRNA complexes. A nucleic acid vector encoding CasΦ.12 fused to VPR, a CasΦ.12 gRNA, and the EGFP reporter were introduced to cells via lipofection and EGFP expression was quantified by flow cytometry. Flow cytometry quantification showed that EGFP expression was 20% to 40% greater in eukaryotic, mammalian HEK293T cells that received the nucleic acid vector encoding the CasΦ.12-VPR fusion and a gRNA relative to negative control (CasΦ.12-VPR fusion with a non-targeting guide RNA).

Next, multiple gene targets, including HBG1, ASCL1, INS and NEUROD1, were selected for testing the ability of CasΦ.12-VPR fusions to increase endogenous gene expression. A nucleic acid vector encoding CasΦ.12 fused to VPR and at least one CasΦ.12 gRNA targeting an endogenous gene were introduced to cells via lipofection. Relative amounts of RNA, indicative of relative gene expression, were quantified with RT-qPCR. An increase of gene expression was observed with individual and pooled gRNAs.

### Example 3: Reduction of Gene Expression with CasΦ Fusion (CRISPRi)

An EGFP reporter was generated with a pSV40 promoter that drove constitutive expression of EGFP. A nucleic acid vector encoding CasΦ.12 fused to KRAB, a CasΦ.12 gRNA, and the EGFP reporter were introduced to cells via lipofection and EGFP expression was quantified by flow cytometry. Flow cytometry quantification showed that EGFP expression was reduced by 25% to 35% in cells that received the nucleic acid vector encoding the CasΦ.12-KRAB fusion and a gRNA relative to negative control (cells receiving the nucleic acid vector encoding the CasΦ.12-KRAB fusion without a gRNA).

Next, multiple gene targets, including BRCA1, CXCR4, MAPT, and SNCA, were selected for testing the ability of CasΦ.12-KRAB fusions to reduce endogenous gene expression. A nucleic acid vector encoding CasΦ.12 fused to VPR and at least one CasΦ.12 gRNA targeting an endogenous gene were introduced to cells via lipofection. Relative amounts of RNA, indicative of relative gene expression, were quantified with RT-qPCR. Reduction of gene expression was observed with individual and pooled gRNAs.

### Example 4: Generating a Catalytically Inactive Variant of a CRISPR Cas Effector Protein

Extensive work has been done to evaluate the overall domain structure of the CRISPR Cas enzymes in the last decade. These data can be an effective reference when trying to identify a catalytic residue of a Cas nuclease. By selecting the residue of a Cas nuclease of interest that aligns at the same relative location as the catalytic residue of a known nuclease when the Cas nuclease and known nuclease are aligned for maximal sequence identity, one can identify the catalytic residue of the Cas nuclease.

Typically, catalytic residues of a RuvC domain are a first aspartic acid (D), glutamic acid (E), and a second aspartic acid (D). In the following example, two closely related Cas nuclease sequences, Cas14a.1 (SEQ ID NO: 8), and CasM_19952 (SEQ ID NO: 176), are aligned. Previous structure study has identified the catalytic residue for Cas14a.1 as D326, E422, and D510. Based on the sequence alignment, we can see that those residues are conserved between Cas14a.1 and CasM_19952. As a result, the potential catalytic active residues of CasM_19952 are D267, E363, and D450. Many amino acid replacements of any catalytic residue can inactivate the nuclease. The most common mutations are converting these residues to alanine or to other amino acids that substitute the acid side chain while maintaining the structural similarity, e.g., such as D (aspartate) to N (asparagine), or E (glutamate) to Q (glutamine). For example, D267A, E363A, D450A, D267N, E363Q, D450N are all potential catalytically dead mutants of CasM_19952.

Sequence or structural analogs of a Cas nuclease provide an additional or supplemental way to predict the catalytic residues of the novel Cas nuclease relative to the previous description in this Example. For example, CasM_19952 was aligned with several structural analogs. Based on the resulting multiple sequence alignment, 14 different amino acids were identified that are over 99% conserved across these different proteins. This number might be different in each case, but catalytic residues are usually highly conserved and can be identified in this manner. Among these amino acids, there were two aspartic acids and one glutamic acid. Given that DED are the typical catalytic residues for RuvC domains, a simple interpretation will be these three residues are the catalytic residues. Another piece of information that can be helpful to identify the catalytic residues of a RuvC domain is that the first aspartic acids of the catalytic residues are typically flanked by the sequence, GXDXG (SEQ ID NO: 542), wherein X is any amino acid. This method is particularly useful for novel Cas variants with a large number of diverse analogs.

Alternatively, or additionally to the description already provided in this Example, computational software may be used to predict the structure of a Cas nuclease. Since the RuvC domains have similar structural compositions, it is also a useful tool to help provide an understanding of the location of the catalytic residues.

### Example 5: Base Editing with Dead CasΦ12 Variants-Deaminase Fusion Proteins

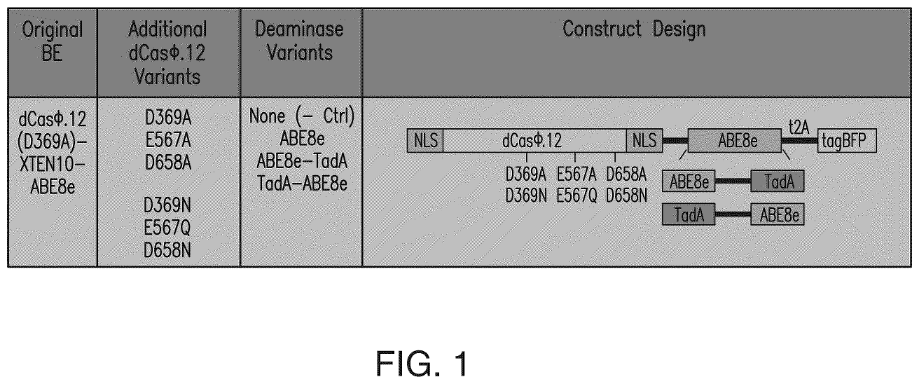

Further to the experiments described in Example 1, multiple nucleic acid vectors encoding additional CasΦ.12 fusion proteins (SEQ ID NOS: 543-559) were constructed as shown in FIG. 1 and assessed for base editing activity. These additional fusion proteins comprised additional catalytically inactive variants of the active CasΦ.12 (SEQ ID NO: 6), also referred to as “dead CasΦ.12” variants (E567A, D658A, D369N, E567Q and D658N) and were fused to either ABE8e (SEQ ID NO: 400), ABE8e-TadA (SEQ ID NO: 563), or TadA-ABE8e (SEQ ID NO: 564), via an XTEN10 linker (SEQ ID NO: 513) as described in Example 1. Like Example 1, these vectors encoded an amino acid sequence containing a nuclear localization signal (MPKKKRKVGIHGVPAA; SEQ ID NO: 603) fused to the dead CasΦ12, but did not encode a uracil glycosylase inhibitor (UGI). Unfused dead CasΦ12 catalytic mutant effector proteins served as negative controls comprising no deaminase, or base editing function. The amino acid sequences of the further modified fusion proteins are provided in TABLE 6. Target sequences included sequences located in the genes for B2M, TRAC, FUT8, or PDCD1. Guide RNA sequences targeting 4 base editing sites were selected from SEQ ID NO: 537, SEQ ID NO: 535, SEQ ID NO: 540, and SEQ ID NO: 541 (or corresponding RNA sequences of SEQ ID NO: 778, SEQ ID NO: 776, SEQ ID NO: 781, and SEQ ID NO: 782), respectively, and are provided in TABLE 5. Cells were treated and base modifications were analyzed according to Example 1. A-to-G editing was observed 3′ of the target site, surrounding position 5. For reference, the last base in the PAM is position −1 and the first base after the PAM is position 1. In this case, for CasΦ.12, the PAM is NTTN from positions −4 to −1.

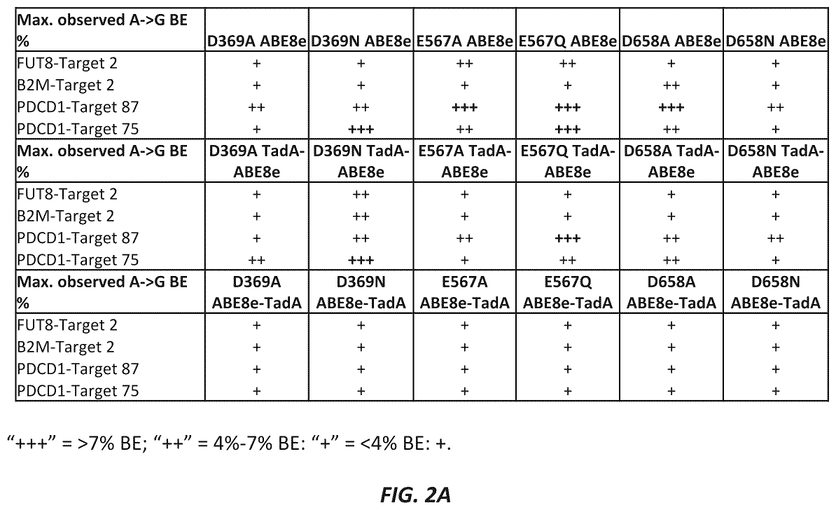

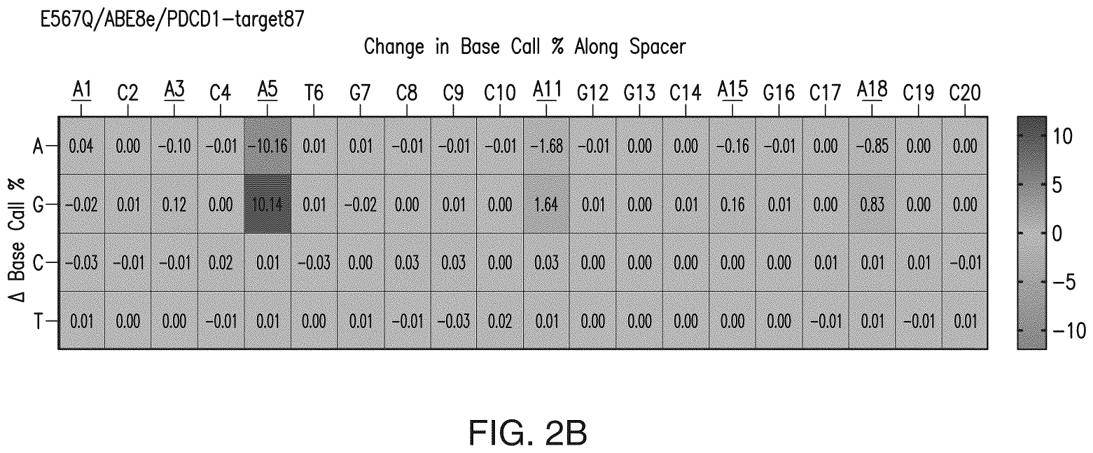

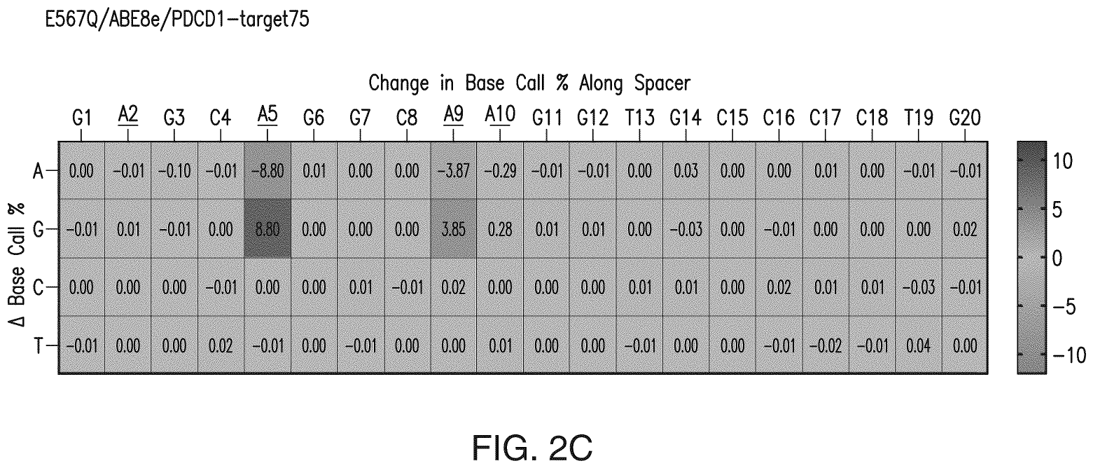

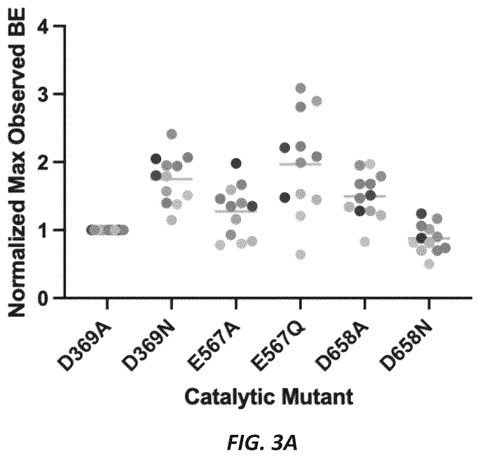

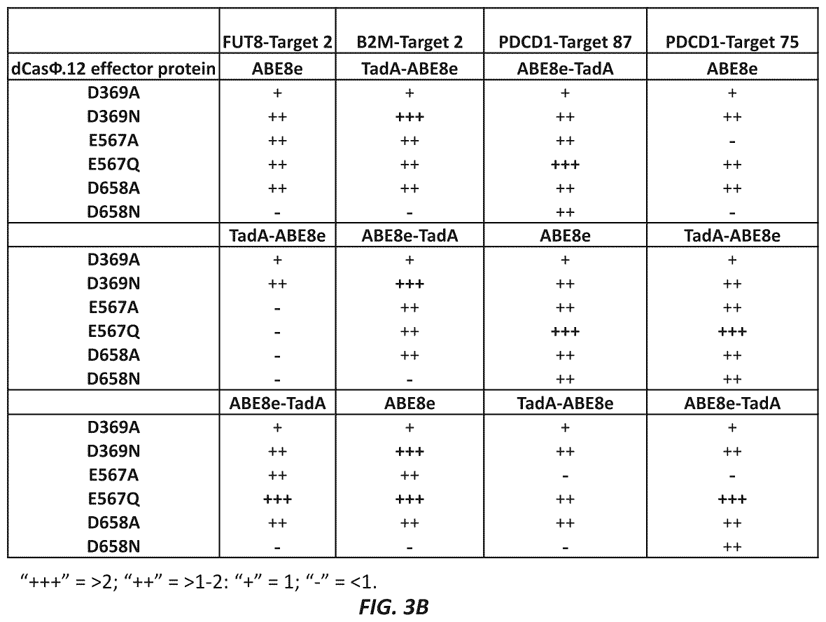

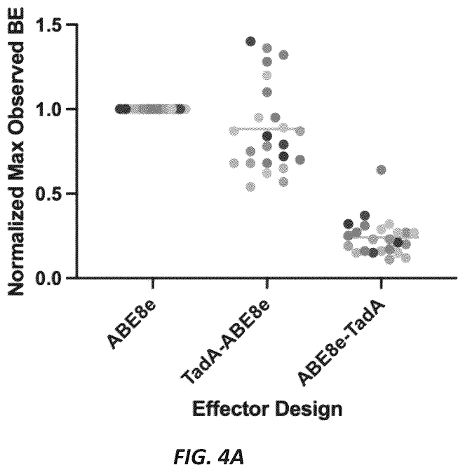

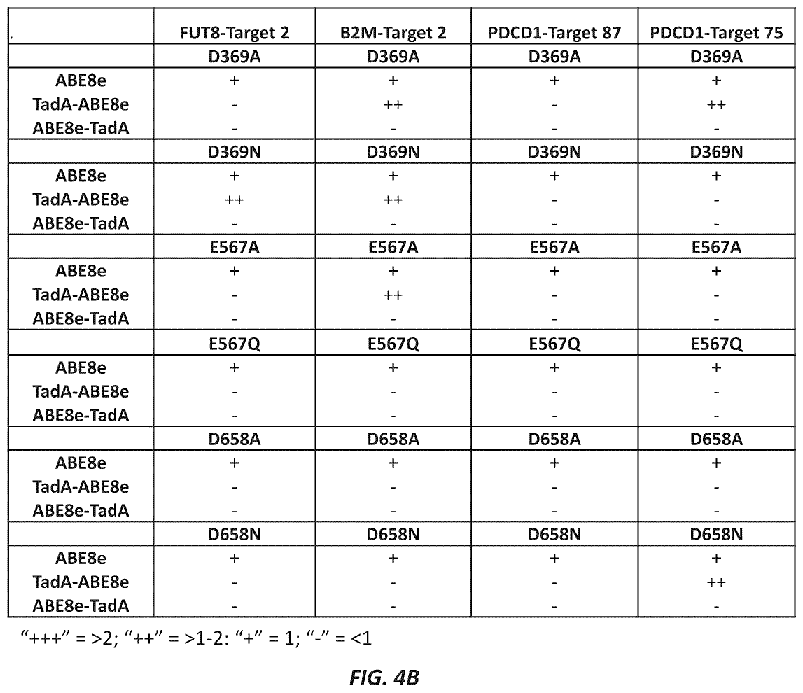

Maximum observed base editing for A to G was quantified for all variants tested at each of the 4 gRNA target sites described above as shown in FIG. 2A. Results were recorded as a change in % base call relative to the negative control. Two of the four gRNAs demonstrated >8% adenine to guanine editing in the spacer of the non-template (coding) strand as shown in FIG. 2B and FIG. 2C, respectively. Editing window centered around position 5 and extended toward the 3′ direction with diminishing effect. Minimal or no editing occurred on the position 4 or earlier. No editing was observed in the 10 bases immediately preceding and following the 5′ and 3′ ends of the spacer, respectively. Cas variants with stronger DNA binding resulted in improved base editing outcomes. Base editing efficacy of the various fusion proteins by effector protein catalytic mutation is illustrated in FIG. 3A. D369N and E567Q mutants demonstrated approximate 2-fold increases in normalized maximum observed base editing. Binned normalized maximum observed base editing is shown in FIG. 3B. Effector design was also analyzed as shown in FIG. 4A comparing the ABE8e monomer with the deaminase dimers, ABE8e-TadA and TadA-ABE8e. Abe8e demonstrated highest base editing efficacy on average. TadA fused at the carboxy terminus (ABE8e-TadA) demonstrated inferior base editing efficacy across the different catalytic mutant fusion proteins tested. Binned normalized maximum observed base editing is shown in FIG. 4B.

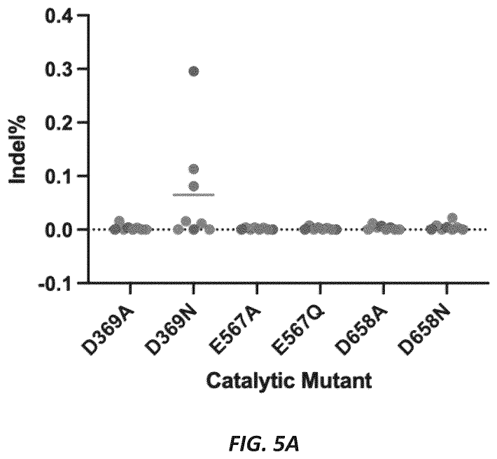

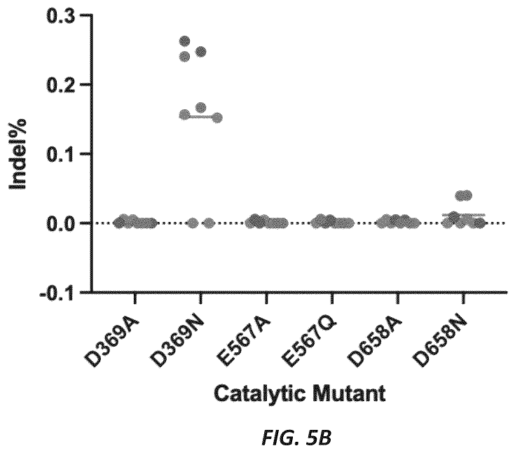

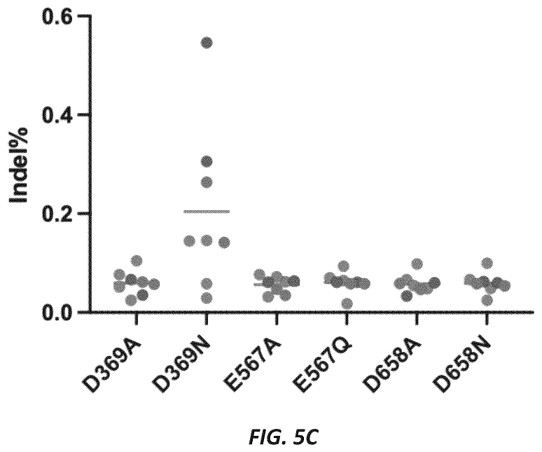

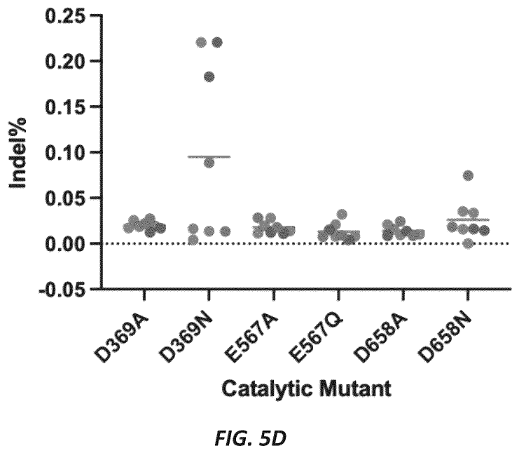

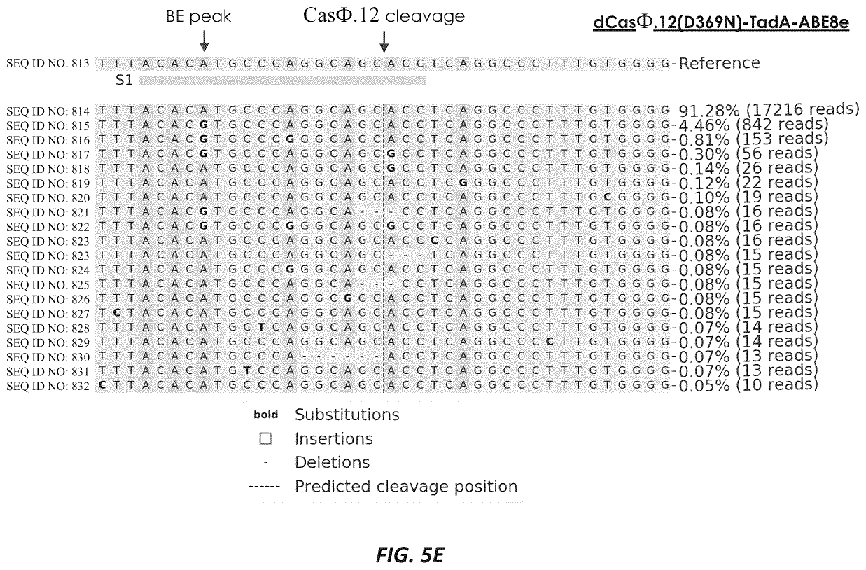

Indel occurrence for each dCasΦ.12 fusion protein variant (SEQ ID NOS: 530, 543-559) was also analyzed for each base editor gRNA. ABE-fused fusion protein variants show detectable indel occurrence as shown in FIG. 5A-D. However, all variants showed low indel occurrence which reflects successful editing with little to no undesired indel occurrence. The D369N mutant had the highest indel occurrence for all four editing targets. Indel occurrence was evaluated for all fusion effector protein and control effector proteins (note comprising a fusion partner acting as a base editor). However, all variants showed low indel occurrence, which reflects successful editing with little undesired indel occurrence. Exemplary target sequences are shown in FIG. 5E (SEQ ID NOS: 813-832). Indel occurrence was observed near the effector protein cleavage site and was not observed at or near the base editing window as shown in FIG. 5E. This demonstrates that indel occurrence is likely associated with the effector protein mutation and not the fusion partner. In comparing the D369A and E567Q mutants, E567Q mutants had lower indel occurrence, demonstrating E567Q mutants have a more inert nuclease profile.

This method allowed for the identification of alternative catalytic mutants to the effector protein of the fusion proteins for enhanced base editing activity.

TABLE 6
dCasΦ.12 Base Editor Fusion Proteins

| Fusion | |
|---|---|
| Name | Amino Acid Sequence |
| dCasΦ.12 | MIKPTVSQFLTPGFKLIRNHSRTAGLKLKNEGEEACKKFVRENEIPKDE |
| (D369N)- | CPNFQGGPAIANIIAKSREFTEWEIYQSSLAIQEVIFTLPKDKLPEPILKEE |
| XTEN10- | WRAQWLSEHGLDTVPYKEAAGLNLIIKNAVNTYKGVQVKVDNKNKN |
| ABE8e | NLAKINRKNEIAKLNGEQEISFEEIKAFDDKGYLLQKPSPNKSIYCYQSV |
| | SPKPFITSKYHNVNLPEEYIGYYRKSNEPIVSPYQFDRLRIPIGEPGYVPK |
| | WQYTFLSKKENKRRKLSKRIKNVSPILGIICIKKDWCVFDMRGLLRTNH |
| | WKKYHKPTDSINDLFDYFTGDPVIDTKANVVRFRYKMENGIVNYKPV |
| | REKKGKELLENICDQNGSCKLATVNVGQNNPVAIGLFELKKVNGELTK |
| | TLISRHPTPIDFCNKITAYRERYDKLESSIKLDAIKQLTSEQKIEVDNYNN |
| | NFTPQNTKQIVCSKLNINPNDLPWDKMISGTHFISEKAQVSNKSEIYFTS |
| | TDKGKTKDVMKSDYKWFQDYKPKLSKEVRDALSDIEWRLRRESLEFN |
| | KLSKSREQDARQLANWISSMCDVIGIENLVKKNNFFGGSGKREPGWD |
| | NFYKPKKENRWWINAIHKALTELSQNKGKRVILLPAMRTSITCPKCKY |
| | CDSKNRNGEKFNCLKCGIELNADIDVATENLATVAITAQSMPKPTCER |
| | SGDAKKPVRARKAKAPEFHDKLAPSYTVVLREAVKRPAATKKAGQA |
| | KKKKGSGSPAGSPTSTRSGGGSGTSSEVEFSHEYWMRHALTLAKRAR |
| | DEREVPVGAVLVLNNRVIGEGWNRAIGLHDPTAHAEIMALRQGGLVM |
| | QNYRLIDATLYVTFEPCVMCAGAMIHSRIGRVVFGVRNSKRGAAGSL |
| | MNVLNYPGMNHRVEITEGILADECAALLCDFYRMPRQVFNAQKKAQS |
| | SIN (SEQ ID NO: 543) |
| dCasΦ.12 | MIKPTVSQFLTPGFKLIRNHSRTAGLKLKNEGEEACKKFVRENEIPKDE |
| (E567A)- | CPNFQGGPAIANIIAKSREFTEWEIYQSSLAIQEVIFTLPKDKLPEPILKEE |
| XTEN10- | WRAQWLSEHGLDTVPYKEAAGLNLIIKNAVNTYKGVQVKVDNKNKN |
| ABE8e | NLAKINRKNEIAKLNGEQEISFEEIKAFDDKGYLLQKPSPNKSIYCYQSV |
| | SPKPFITSKYHNVNLPEEYIGYYRKSNEPIVSPYQFDRLRIPIGEPGYVPK |
| | WQYTFLSKKENKRRKLSKRIKNVSPILGIICIKKDWCVFDMRGLLRTNH |
| | WKKYHKPTDSINDLFDYFTGDPVIDTKANVVRFRYKMENGIVNYKPV |
| | REKKGKELLENICDQNGSCKLATVDVGQNNPVAIGLFELKKVNGELTK |
| | TLISRHPTPIDFCNKITAYRERYDKLESSIKLDAIKQLTSEQKIEVDNYNN |
| | NFTPQNTKQIVCSKLNINPNDLPWDKMISGTHFISEKAQVSNKSEIYFTS |
| | TDKGKTKDVMKSDYKWFQDYKPKLSKEVRDALSDIEWRLRRESLEFN |
| | KLSKSREQDARQLANWISSMCDVIGIANLVKKNNFFGGSGKREPGWD |
| | NFYKPKKENRWWINAIHKALTELSQNKGKRVILLPAMRTSITCPKCKY |
| | CDSKNRNGEKFNCLKCGIELNADIDVATENLATVAITAQSMPKPTCER |
| | SGDAKKPVRARKAKAPEFHDKLAPSYTVVLREAVKRPAATKKAGQA |
| | KKKKGSGSPAGSPTSTRSGGGSGTSSEVEFSHEYWMRHALTLAKRAR |
| | DEREVPVGAVLVLNNRVIGEGWNRAIGLHDPTAHAEIMALRQGGLVM |
| | QNYRLIDATLYVTFEPCVMCAGAMIHSRIGRVVFGVRNSKRGAAGSL |
| | MNVLNYPGMNHRVEITEGILADECAALLCDFYRMPRQVFNAQKKAQS |
| | SIN (SEQ ID NO: 544) |
| dCasΦ.12 | MIKPTVSQFLTPGFKLIRNHSRTAGLKLKNEGEEACKKFVRENEIPKDE |
| (E567Q)- | CPNFQGGPAIANIIAKSREFTEWEIYQSSLAIQEVIFTLPKDKLPEPILKEE |
| XTEN10- | WRAQWLSEHGLDTVPYKEAAGLNLIIKNAVNTYKGVQVKVDNKNKN |
| ABE8e | NLAKINRKNEIAKLNGEQEISFEEIKAFDDKGYLLQKPSPNKSIYCYQSV |
| | SPKPFITSKYHNVNLPEEYIGYYRKSNEPIVSPYQFDRLRIPIGEPGYVPK |
| | WQYTFLSKKENKRRKLSKRIKNVSPILGIICIKKDWCVFDMRGLLRTNH |
| | WKKYHKPTDSINDLFDYFTGDPVIDTKANVVRFRYKMENGIVNYKPV |
| | REKKGKELLENICDQNGSCKLATVDVGQNNPVAIGLFELKKVNGELTK |
| | TLISRHPTPIDFCNKITAYRERYDKLESSIKLDAIKQLTSEQKIEVDNYNN |
| | NFTPQNTKQIVCSKLNINPNDLPWDKMISGTHFISEKAQVSNKSEIYFTS |
| | TDKGKTKDVMKSDYKWFQDYKPKLSKEVRDALSDIEWRLRRESLEFN |
| | KLSKSREQDARQLANWISSMCDVIGIQNLVKKNNFFGGSGKREPGWD |
| | NFYKPKKENRWWINAIHKALTELSQNKGKRVILLPAMRTSITCPKCKY |
| | CDSKNRNGEKFNCLKCGIELNADIDVATENLATVAITAQSMPKPTCER |
| | SGDAKKPVRARKAKAPEFHDKLAPSYTVVLREAVKRPAATKKAGQA |
| | KKKKGSGSPAGSPTSTRSGGGSGTSSEVEFSHEYWMRHALTLAKRAR |
| | DEREVPVGAVLVLNNRVIGEGWNRAIGLHDPTAHAEIMALRQGGLVM |
| | QNYRLIDATLYVTFEPCVMCAGAMIHSRIGRVVFGVRNSKRGAAGSL |
| | MNVLNYPGMNHRVEITEGILADECAALLCDFYRMPRQVFNAQKKAQS |
| | SIN (SEQ ID NO: 545) |
| dCasΦ.12 | MIKPTVSQFLTPGFKLIRNHSRTAGLKLKNEGEEACKKFVRENEIPKDE |
| (D658A)- | CPNFQGGPAIANIIAKSREFTEWEIYQSSLAIQEVIFTLPKDKLPEPILKEE |
| XTEN10- | WRAQWLSEHGLDTVPYKEAAGLNLIIKNAVNTYKGVQVKVDNKNKN |
| ABE8e | NLAKINRKNEIAKLNGEQEISFEEIKAFDDKGYLLQKPSPNKSIYCYQSV |
| | SPKPFITSKYHNVNLPEEYIGYYRKSNEPIVSPYQFDRLRIPIGEPGYVPK |
| | WQYTFLSKKENKRRKLSKRIKNVSPILGIICIKKDWCVFDMRGLLRTNH |
| | WKKYHKPTDSINDLFDYFTGDPVIDTKANVVRFRYKMENGIVNYKPV |
| | REKKGKELLENICDQNGSCKLATVDVGQNNPVAIGLFELKKVNGELTK |
| | TLISRHPTPIDFCNKITAYRERYDKLESSIKLDAIKQLTSEQKIEVDNYNN |
| | NFTPQNTKQIVCSKLNINPNDLPWDKMISGTHFISEKAQVSNKSEIYFTS |
| | TDKGKTKDVMKSDYKWFQDYKPKLSKEVRDALSDIEWRLRRESLEFN |
| | KLSKSREQDARQLANWISSMCDVIGIENLVKKNNFFGGSGKREPGWD |
| | NFYKPKKENRWWINAIHKALTELSQNKGKRVILLPAMRTSITCPKCKY |
| | CDSKNRNGEKFNCLKCGIELNAAIDVATENLATVAITAQSMPKPTCER |
| | SGDAKKPVRARKAKAPEFHDKLAPSYTVVLREAVKRPAATKKAGQA |
| | KKKKGSGSPAGSPTSTRSGGGSGTSSEVEFSHEYWMRHALTLAKRAR |
| | DEREVPVGAVLVLNNRVIGEGWNRAIGLHDPTAHAEIMALRQGGLVM |
| | QNYRLIDATLYVTFEPCVMCAGAMIHSRIGRVVFGVRNSKRGAAGSL |
| | MNVLNYPGMNHRVEITEGILADECAALLCDFYRMPRQVFNAQKKAQS |
| | SIN (SEQ ID NO: 546) |
| dCasΦ.12 | MIKPTVSQFLTPGFKLIRNHSRTAGLKLKNEGEEACKKFVRENEIPKDE |
| (D658N)- | CPNFQGGPAIANIIAKSREFTEWEIYQSSLAIQEVIFTLPKDKLPEPILKEE |
| XTEN10- | WRAQWLSEHGLDTVPYKEAAGLNLIIKNAVNTYKGVQVKVDNKNKN |
| ABE8e | NLAKINRKNEIAKLNGEQEISFEEIKAFDDKGYLLQKPSPNKSIYCYQSV |
| | SPKPFITSKYHNVNLPEEYIGYYRKSNEPIVSPYQFDRLRIPIGEPGYVPK |
| | WQYTFLSKKENKRRKLSKRIKNVSPILGIICIKKDWCVFDMRGLLRTNH |
| | WKKYHKPTDSINDLFDYFTGDPVIDTKANVVRFRYKMENGIVNYKPV |
| | REKKGKELLENICDQNGSCKLATVDVGQNNPVAIGLFELKKVNGELTK |
| | TLISRHPTPIDFCNKITAYRERYDKLESSIKLDAIKQLTSEQKIEVDNYNN |
| | NFTPQNTKQIVCSKLNINPNDLPWDKMISGTHFISEKAQVSNKSEIYFTS |
| | TDKGKTKDVMKSDYKWFQDYKPKLSKEVRDALSDIEWRLRRESLEFN |
| | KLSKSREQDARQLANWISSMCDVIGIENLVKKNNFFGGSGKREPGWD |
| | NFYKPKKENRWWINAIHKALTELSQNKGKRVILLPAMRTSITCPKCKY |
| | CDSKNRNGEKFNCLKCGIELNANIDVATENLATVAITAQSMPKPTCER |
| | SGDAKKPVRARKAKAPEFHDKLAPSYTVVLREAVKRPAATKKAGQA |
| | KKKKGSGSPAGSPTSTRSGGGSGTSSEVEFSHEYWMRHALTLAKRAR |
| | DEREVPVGAVLVLNNRVIGEGWNRAIGLHDPTAHAEIMALRQGGLVM |
| | QNYRLIDATLYVTFEPCVMCAGAMIHSRIGRVVFGVRNSKRGAAGSL |
| | MNVLNYPGMNHRVEITEGILADECAALLCDFYRMPRQVFNAQKKAQS |
| | SIN (SEQ ID NO: 547) |
| dCasΦ.12 | MIKPTVSQFLTPGFKLIRNHSRTAGLKLKNEGEEACKKFVRENEIPKDE |
| (D369A)- | CPNFQGGPAIANIIAKSREFTEWEIYQSSLAIQEVIFTLPKDKLPEPILKEE |
| XTEN10- | WRAQWLSEHGLDTVPYKEAAGLNLIIKNAVNTYKGVQVKVDNKNKN |
| TadA- | NLAKINRKNEIAKLNGEQEISFEEIKAFDDKGYLLQKPSPNKSIYCYQSV |
| ABE8e | SPKPFITSKYHNVNLPEEYIGYYRKSNEPIVSPYQFDRLRIPIGEPGYVPK |
| | WQYTFLSKKENKRRKLSKRIKNVSPILGIICIKKDWCVFDMRGLLRTNH |
| | WKKYHKPTDSINDLFDYFTGDPVIDTKANVVRFRYKMENGIVNYKPV |
| | REKKGKELLENICDQNGSCKLATVAVGQNNPVAIGLFELKKVNGELTK |
| | TLISRHPTPIDFCNKITAYRERYDKLESSIKLDAIKQLTSEQKIEVDNYNN |
| | NFTPQNTKQIVCSKLNINPNDLPWDKMISGTHFISEKAQVSNKSEIYFTS |
| | TDKGKTKDVMKSDYKWFQDYKPKLSKEVRDALSDIEWRLRRESLEFN |
| | KLSKSREQDARQLANWISSMCDVIGIENLVKKNNFFGGSGKREPGWD |
| | NFYKPKKENRWWINAIHKALTELSQNKGKRVILLPAMRTSITCPKCKY |
| | CDSKNRNGEKFNCLKCGIELNADIDVATENLATVAITAQSMPKPTCER |
| | SGDAKKPVRARKAKAPEFHDKLAPSYTVVLREAVKRPAATKKAGQA |
| | KKKKGSGSPAGSPTSTRSGGGSGTSSEVEFSHEYWMRHALTLAKRAW |
| | DEREVPVGAVLVHNNRVIGEGWNRPIGRHDPTAHAEIMALRQGGLVM |
| | QNYRLIDATLYVTLEPCVMCAGAMIHSRIGRVVFGARDAKTGAAGSL |
| | MDVLHHPGMNHRVEITEGILADECAALLSDFFRMRRQEIKAQKKAQSS |
| | TDSGGSSGGSSGSETPGTSESATPESSGGSSGGSSEVEFSHEYWMRHAL |
| | TLAKRARDEREVPVGAVLVLNNRVIGEGWNRAIGLHDPTAHAEIMAL |
| | RQGGLVMQNYRLIDATLYVTFEPCVMCAGAMIHSRIGRVVFGVRNSK |
| | RGAAGSLMNVLNYPGMNHRVEITEGILADECAALLCDFYRMPRQVEN |
| | AQKKAQSSIN (SEQ ID NO: 548) |
| dCasΦ.12 | MIKPTVSQFLTPGFKLIRNHSRTAGLKLKNEGEEACKKFVRENEIPKDE |
| (D369N)- | CPNFQGGPAIANIIAKSREFTEWEIYQSSLAIQEVIFTLPKDKLPEPILKEE |
| XTEN10- | WRAQWLSEHGLDTVPYKEAAGLNLIIKNAVNTYKGVQVKVDNKNKN |
| TadA- | NLAKINRKNEIAKLNGEQEISFEEIKAFDDKGYLLQKPSPNKSIYCYQSV |
| ABE8e | SPKPFITSKYHNVNLPEEYIGYYRKSNEPIVSPYQFDRLRIPIGEPGYVPK |
| | WQYTFLSKKENKRRKLSKRIKNVSPILGIICIKKDWCVFDMRGLLRTNH |
| | WKKYHKPTDSINDLFDYFTGDPVIDTKANVVRFRYKMENGIVNYKPV |
| | REKKGKELLENICDQNGSCKLATVNVGQNNPVAIGLFELKKVNGELTK |
| | TLISRHPTPIDFCNKITAYRERYDKLESSIKLDAIKQLTSEQKIEVDNYNN |
| | NFTPQNTKQIVCSKLNINPNDLPWDKMISGTHFISEKAQVSNKSEIYFTS |
| | TDKGKTKDVMKSDYKWFQDYKPKLSKEVRDALSDIEWRLRRESLEFN |
| | KLSKSREQDARQLANWISSMCDVIGIENLVKKNNFFGGSGKREPGWD |
| | NFYKPKKENRWWINAIHKALTELSQNKGKRVILLPAMRTSITCPKCKY |
| | CDSKNRNGEKFNCLKCGIELNADIDVATENLATVAITAQSMPKPTCER |
| | SGDAKKPVRARKAKAPEFHDKLAPSYTVVLREAVKRPAATKKAGQA |
| | KKKKGSGSPAGSPTSTRSGGGSGTSSEVEFSHEYWMRHALTLAKRAW |
| | DEREVPVGAVLVHNNRVIGEGWNRPIGRHDPTAHAEIMALRQGGLVM |
| | QNYRLIDATLYVTLEPCVMCAGAMIHSRIGRVVFGARDAKTGAAGSL |
| | MDVLHHPGMNHRVEITEGILADECAALLSDFFRMRRQEIKAQKKAQSS |
| | TDSGGSSGGSSGSETPGTSESATPESSGGSSGGSSEVEFSHEYWMRHAL |
| | TLAKRARDEREVPVGAVLVLNNRVIGEGWNRAIGLHDPTAHAEIMAL |
| | RQGGLVMQNYRLIDATLYVTFEPCVMCAGAMIHSRIGRVVFGVRNSK |
| | RGAAGSLMNVLNYPGMNHRVEITEGILADECAALLCDFYRMPRQVEN |
| | AQKKAQSSIN (SEQ ID NO: 549) |
| dCasΦ.12 | MIKPTVSQFLTPGFKLIRNHSRTAGLKLKNEGEEACKKFVRENEIPKDE |
| (E567A)- | CPNFQGGPAIANIIAKSREFTEWEIYQSSLAIQEVIFTLPKDKLPEPILKEE |
| XTEN10- | WRAQWLSEHGLDTVPYKEAAGLNLIIKNAVNTYKGVQVKVDNKNKN |
| TadA- | NLAKINRKNEIAKLNGEQEISFEEIKAFDDKGYLLQKPSPNKSIYCYQSV |
| ABE8e | SPKPFITSKYHNVNLPEEYIGYYRKSNEPIVSPYQFDRLRIPIGEPGYVPK |
| | WQYTFLSKKENKRRKLSKRIKNVSPILGIICIKKDWCVFDMRGLLRTNH |
| | WKKYHKPTDSINDLFDYFTGDPVIDTKANVVRFRYKMENGIVNYKPV |
| | REKKGKELLENICDQNGSCKLATVDVGQNNPVAIGLFELKKVNGELTK |
| | TLISRHPTPIDFCNKITAYRERYDKLESSIKLDAIKQLTSEQKIEVDNYNN |
| | NFTPQNTKQIVCSKLNINPNDLPWDKMISGTHFISEKAQVSNKSEIYFTS |
| | TDKGKTKDVMKSDYKWFQDYKPKLSKEVRDALSDIEWRLRRESLEFN |
| | KLSKSREQDARQLANWISSMCDVIGIANLVKKNNFFGGSGKREPGWD |
| | NFYKPKKENRWWINAIHKALTELSQNKGKRVILLPAMRTSITCPKCKY |
| | CDSKNRNGEKFNCLKCGIELNADIDVATENLATVAITAQSMPKPTCER |
| | SGDAKKPVRARKAKAPEFHDKLAPSYTVVLREAVKRPAATKKAGQA |
| | KKKKGSGSPAGSPTSTRSGGGSGTSSEVEFSHEYWMRHALTLAKRAW |
| | DEREVPVGAVLVHNNRVIGEGWNRPIGRHDPTAHAEIMALRQGGLVM |
| | QNYRLIDATLYVTLEPCVMCAGAMIHSRIGRVVFGARDAKTGAAGSL |
| | MDVLHHPGMNHRVEITEGILADECAALLSDFFRMRRQEIKAQKKAQSS |
| | TDSGGSSGGSSGSETPGTSESATPESSGGSSGGSSEVEFSHEYWMRHAL |
| | TLAKRARDEREVPVGAVLVLNNRVIGEGWNRAIGLHDPTAHAEIMAL |
| | RQGGLVMQNYRLIDATLYVTFEPCVMCAGAMIHSRIGRVVFGVRNSK |
| | RGAAGSLMNVLNYPGMNHRVEITEGILADECAALLCDFYRMPRQVEN |
| | AQKKAQSSIN (SEQ ID NO: 550) |
| dCasΦ.12 | MIKPTVSQFLTPGFKLIRNHSRTAGLKLKNEGEEACKKFVRENEIPKDE |
| (E567Q)- | CPNFQGGPAIANIIAKSREFTEWEIYQSSLAIQEVIFTLPKDKLPEPILKEE |
| XTEN10- | WRAQWLSEHGLDTVPYKEAAGLNLIIKNAVNTYKGVQVKVDNKNKN |
| TadA- | NLAKINRKNEIAKLNGEQEISFEEIKAFDDKGYLLQKPSPNKSIYCYQSV |
| ABE8e | SPKPFITSKYHNVNLPEEYIGYYRKSNEPIVSPYQFDRLRIPIGEPGYVPK |
| | WQYTFLSKKENKRRKLSKRIKNVSPILGIICIKKDWCVFDMRGLLRTNH |
| | WKKYHKPTDSINDLFDYFTGDPVIDTKANVVRFRYKMENGIVNYKPV |
| | REKKGKELLENICDQNGSCKLATVDVGQNNPVAIGLFELKKVNGELTK |
| | TLISRHPTPIDFCNKITAYRERYDKLESSIKLDAIKQLTSEQKIEVDNYNN |
| | NFTPQNTKQIVCSKLNINPNDLPWDKMISGTHFISEKAQVSNKSEIYFTS |
| | TDKGKTKDVMKSDYKWFQDYKPKLSKEVRDALSDIEWRLRRESLEFN |
| | KLSKSREQDARQLANWISSMCDVIGIQNLVKKNNFFGGSGKREPGWD |
| | NFYKPKKENRWWINAIHKALTELSQNKGKRVILLPAMRTSITCPKCKY |
| | CDSKNRNGEKFNCLKCGIELNADIDVATENLATVAITAQSMPKPTCER |
| | SGDAKKPVRARKAKAPEFHDKLAPSYTVVLREAVKRPAATKKAGQA |
| | KKKKGSGSPAGSPTSTRSGGGSGTSSEVEFSHEYWMRHALTLAKRAW |
| | DEREVPVGAVLVHNNRVIGEGWNRPIGRHDPTAHAEIMALRQGGLVM |
| | QNYRLIDATLYVTLEPCVMCAGAMIHSRIGRVVFGARDAKTGAAGSL |
| | MDVLHHPGMNHRVEITEGILADECAALLSDFFRMRRQEIKAQKKAQSS |
| | TDSGGSSGGSSGSETPGTSESATPESSGGSSGGSSEVEFSHEYWMRHAL |
| | TLAKRARDEREVPVGAVLVLNNRVIGEGWNRAIGLHDPTAHAEIMAL |
| | RQGGLVMQNYRLIDATLYVTFEPCVMCAGAMIHSRIGRVVFGVRNSK |
| | RGAAGSLMNVLNYPGMNHRVEITEGILADECAALLCDFYRMPRQVEN |
| | AQKKAQSSIN (SEQ ID NO: 551) |
| dCasΦ.12 | MIKPTVSQFLTPGFKLIRNHSRTAGLKLKNEGEEACKKFVRENEIPKDE |
| (D658A)- | CPNFQGGPAIANIIAKSREFTEWEIYQSSLAIQEVIFTLPKDKLPEPILKEE |
| XTEN10- | WRAQWLSEHGLDTVPYKEAAGLNLIIKNAVNTYKGVQVKVDNKNKN |
| TadA- | NLAKINRKNEIAKLNGEQEISFEEIKAFDDKGYLLQKPSPNKSIYCYQSV |
| ABE8e | SPKPFITSKYHNVNLPEEYIGYYRKSNEPIVSPYQFDRLRIPIGEPGYVPK |
| | WQYTFLSKKENKRRKLSKRIKNVSPILGIICIKKDWCVFDMRGLLRTNH |
| | WKKYHKPTDSINDLFDYFTGDPVIDTKANVVRFRYKMENGIVNYKPV |
| | REKKGKELLENICDQNGSCKLATVDVGQNNPVAIGLFELKKVNGELTK |
| | TLISRHPTPIDFCNKITAYRERYDKLESSIKLDAIKQLTSEQKIEVDNYNN |
| | NFTPQNTKQIVCSKLNINPNDLPWDKMISGTHFISEKAQVSNKSEIYFTS |
| | TDKGKTKDVMKSDYKWFQDYKPKLSKEVRDALSDIEWRLRRESLEFN |
| | KLSKSREQDARQLANWISSMCDVIGIENLVKKNNFFGGSGKREPGWD |
| | NFYKPKKENRWWINAIHKALTELSQNKGKRVILLPAMRTSITCPKCKY |
| | CDSKNRNGEKFNCLKCGIELNAAIDVATENLATVAITAQSMPKPTCER |
| | SGDAKKPVRARKAKAPEFHDKLAPSYTVVLREAVKRPAATKKAGQA |
| | KKKKGSGSPAGSPTSTRSGGGSGTSSEVEFSHEYWMRHALTLAKRAW |
| | DEREVPVGAVLVHNNRVIGEGWNRPIGRHDPTAHAEIMALRQGGLVM |
| | QNYRLIDATLYVTLEPCVMCAGAMIHSRIGRVVFGARDAKTGAAGSL |
| | MDVLHHPGMNHRVEITEGILADECAALLSDFFRMRRQEIKAQKKAQSS |
| | TDSGGSSGGSSGSETPGTSESATPESSGGSSGGSSEVEFSHEYWMRHAL |
| | TLAKRARDEREVPVGAVLVLNNRVIGEGWNRAIGLHDPTAHAEIMAL |
| | RQGGLVMQNYRLIDATLYVTFEPCVMCAGAMIHSRIGRVVFGVRNSK |
| | RGAAGSLMNVLNYPGMNHRVEITEGILADECAALLCDFYRMPRQVEN |
| | AQKKAQSSIN (SEQ ID NO: 552) |
| dCasΦ.12 | MIKPTVSQFLTPGFKLIRNHSRTAGLKLKNEGEEACKKFVRENEIPKDE |
| (D658N)- | CPNFQGGPAIANIIAKSREFTEWEIYQSSLAIQEVIFTLPKDKLPEPILKEE |
| XTEN10- | WRAQWLSEHGLDTVPYKEAAGLNLIIKNAVNTYKGVQVKVDNKNKN |
| TadA- | NLAKINRKNEIAKLNGEQEISFEEIKAFDDKGYLLQKPSPNKSIYCYQSV |
| ABE8e | SPKPFITSKYHNVNLPEEYIGYYRKSNEPIVSPYQFDRLRIPIGEPGYVPK |
| | WQYTFLSKKENKRRKLSKRIKNVSPILGIICIKKDWCVFDMRGLLRTNH |
| | WKKYHKPTDSINDLFDYFTGDPVIDTKANVVRFRYKMENGIVNYKPV |
| | REKKGKELLENICDQNGSCKLATVDVGQNNPVAIGLFELKKVNGELTK |
| | TLISRHPTPIDFCNKITAYRERYDKLESSIKLDAIKQLTSEQKIEVDNYNN |
| | NFTPQNTKQIVCSKLNINPNDLPWDKMISGTHFISEKAQVSNKSEIYFTS |
| | TDKGKTKDVMKSDYKWFQDYKPKLSKEVRDALSDIEWRLRRESLEFN |
| | KLSKSREQDARQLANWISSMCDVIGIENLVKKNNFFGGSGKREPGWD |
| | NFYKPKKENRWWINAIHKALTELSQNKGKRVILLPAMRTSITCPKCKY |
| | CDSKNRNGEKFNCLKCGIELNANIDVATENLATVAITAQSMPKPTCER |
| | SGDAKKPVRARKAKAPEFHDKLAPSYTVVLREAVKRPAATKKAGQA |
| | KKKKGSGSPAGSPTSTRSGGGSGTSSEVEFSHEYWMRHALTLAKRAW |
| | DEREVPVGAVLVHNNRVIGEGWNRPIGRHDPTAHAEIMALRQGGLVM |
| | QNYRLIDATLYVTLEPCVMCAGAMIHSRIGRVVFGARDAKTGAAGSL |
| | MDVLHHPGMNHRVEITEGILADECAALLSDFFRMRRQEIKAQKKAQSS |
| | TDSGGSSGGSSGSETPGTSESATPESSGGSSGGSSEVEFSHEYWMRHAL |
| | TLAKRARDEREVPVGAVLVLNNRVIGEGWNRAIGLHDPTAHAEIMAL |
| | RQGGLVMQNYRLIDATLYVTFEPCVMCAGAMIHSRIGRVVFGVRNSK |
| | RGAAGSLMNVLNYPGMNHRVEITEGILADECAALLCDFYRMPRQVEN |
| | AQKKAQSSIN (SEQ ID NO: 553) |
| dCasΦ.12 | MIKPTVSQFLTPGFKLIRNHSRTAGLKLKNEGEEACKKFVRENEIPKDE |
| (D369A)- | CPNFQGGPAIANIIAKSREFTEWEIYQSSLAIQEVIFTLPKDKLPEPILKEE |
| XTEN10- | WRAQWLSEHGLDTVPYKEAAGLNLIIKNAVNTYKGVQVKVDNKNKN |
| ABE8e- | NLAKINRKNEIAKLNGEQEISFEEIKAFDDKGYLLQKPSPNKSIYCYQSV |
| TadA | SPKPFITSKYHNVNLPEEYIGYYRKSNEPIVSPYQFDRLRIPIGEPGYVPK |
| | WQYTFLSKKENKRRKLSKRIKNVSPILGIICIKKDWCVFDMRGLLRTNH |
| | WKKYHKPTDSINDLFDYFTGDPVIDTKANVVRFRYKMENGIVNYKPV |
| | REKKGKELLENICDQNGSCKLATVAVGQNNPVAIGLFELKKVNGELTK |
| | TLISRHPTPIDFCNKITAYRERYDKLESSIKLDAIKQLTSEQKIEVDNYNN |
| | NFTPQNTKQIVCSKLNINPNDLPWDKMISGTHFISEKAQVSNKSEIYFTS |
| | TDKGKTKDVMKSDYKWFQDYKPKLSKEVRDALSDIEWRLRRESLEFN |
| | KLSKSREQDARQLANWISSMCDVIGIENLVKKNNFFGGSGKREPGWD |
| | NFYKPKKENRWWINAIHKALTELSQNKGKRVILLPAMRTSITCPKCKY |
| | CDSKNRNGEKFNCLKCGIELNADIDVATENLATVAITAQSMPKPTCER |
| | SGDAKKPVRARKAKAPEFHDKLAPSYTVVLREAVKRPAATKKAGQA |
| | KKKKGSGSPAGSPTSTRSGGGSGTSSEVEFSHEYWMRHALTLAKRAR |
| | DEREVPVGAVLVLNNRVIGEGWNRAIGLHDPTAHAEIMALRQGGLVM |
| | QNYRLIDATLYVTFEPCVMCAGAMIHSRIGRVVFGVRNSKRGAAGSL |
| | MNVLNYPGMNHRVEITEGILADECAALLCDFYRMPRQVFNAQKKAQS |
| | SINSGGSSGGSSGSETPGTSESATPESSGGSSGGSSEVEFSHEYWMRHAL |
| | TLAKRAWDEREVPVGAVLVHNNRVIGEGWNRPIGRHDPTAHAEIMAL |
| | RQGGLVMQNYRLIDATLYVTLEPCVMCAGAMIHSRIGRVVFGARDAK |
| | TGAAGSLMDVLHHPGMNHRVEITEGILADECAALLSDFFRMRRQEIKA |
| | QKKAQSSTD (SEQ ID NO: 554) |
| dCasΦ.12 | MIKPTVSQFLTPGFKLIRNHSRTAGLKLKNEGEEACKKFVRENEIPKDE |
| (D369N)- | CPNFQGGPAIANIIAKSREFTEWEIYQSSLAIQEVIFTLPKDKLPEPILKEE |
| XTEN10- | WRAQWLSEHGLDTVPYKEAAGLNLIIKNAVNTYKGVQVKVDNKNKN |
| ABE8e- | NLAKINRKNEIAKLNGEQEISFEEIKAFDDKGYLLQKPSPNKSIYCYQSV |
| TadA | SPKPFITSKYHNVNLPEEYIGYYRKSNEPIVSPYQFDRLRIPIGEPGYVPK |
| | WQYTFLSKKENKRRKLSKRIKNVSPILGIICIKKDWCVFDMRGLLRTNH |
| | WKKYHKPTDSINDLFDYFTGDPVIDTKANVVRFRYKMENGIVNYKPV |
| | REKKGKELLENICDQNGSCKLATVNVGQNNPVAIGLFELKKVNGELTK |
| | TLISRHPTPIDFCNKITAYRERYDKLESSIKLDAIKQLTSEQKIEVDNYNN |
| | NFTPQNTKQIVCSKLNINPNDLPWDKMISGTHFISEKAQVSNKSEIYFTS |
| | TDKGKTKDVMKSDYKWFQDYKPKLSKEVRDALSDIEWRLRRESLEFN |
| | KLSKSREQDARQLANWISSMCDVIGIENLVKKNNFFGGSGKREPGWD |
| | NFYKPKKENRWWINAIHKALTELSQNKGKRVILLPAMRTSITCPKCKY |
| | CDSKNRNGEKFNCLKCGIELNADIDVATENLATVAITAQSMPKPTCER |
| | SGDAKKPVRARKAKAPEFHDKLAPSYTVVLREAVKRPAATKKAGQA |
| | KKKKGSGSPAGSPTSTRSGGGSGTSSEVEFSHEYWMRHALTLAKRAR |
| | DEREVPVGAVLVLNNRVIGEGWNRAIGLHDPTAHAEIMALRQGGLVM |
| | QNYRLIDATLYVTFEPCVMCAGAMIHSRIGRVVFGVRNSKRGAAGSL |
| | MNVLNYPGMNHRVEITEGILADECAALLCDFYRMPRQVFNAQKKAQS |
| | SINSGGSSGGSSGSETPGTSESATPESSGGSSGGSSEVEFSHEYWMRHAL |
| | TLAKRAWDEREVPVGAVLVHNNRVIGEGWNRPIGRHDPTAHAEIMAL |
| | RQGGLVMQNYRLIDATLYVTLEPCVMCAGAMIHSRIGRVVFGARDAK |
| | TGAAGSLMDVLHHPGMNHRVEITEGILADECAALLSDFFRMRRQEIKA |
| | QKKAQSSTD (SEQ ID NO: 555) |
| dCasΦ.12 | MIKPTVSQFLTPGFKLIRNHSRTAGLKLKNEGEEACKKFVRENEIPKDE |
| (E567A)- | CPNFQGGPAIANIIAKSREFTEWEIYQSSLAIQEVIFTLPKDKLPEPILKEE |
| XTEN10- | WRAQWLSEHGLDTVPYKEAAGLNLIIKNAVNTYKGVQVKVDNKNKN |
| ABE8e- | NLAKINRKNEIAKLNGEQEISFEEIKAFDDKGYLLQKPSPNKSIYCYQSV |
| TadA | SPKPFITSKYHNVNLPEEYIGYYRKSNEPIVSPYQFDRLRIPIGEPGYVPK |
| | WQYTFLSKKENKRRKLSKRIKNVSPILGIICIKKDWCVFDMRGLLRTNH |
| | WKKYHKPTDSINDLFDYFTGDPVIDTKANVVRFRYKMENGIVNYKPV |
| | REKKGKELLENICDQNGSCKLATVDVGQNNPVAIGLFELKKVNGELTK |
| | TLISRHPTPIDFCNKITAYRERYDKLESSIKLDAIKQLTSEQKIEVDNYNN |
| | NFTPQNTKQIVCSKLNINPNDLPWDKMISGTHFISEKAQVSNKSEIYFTS |
| | TDKGKTKDVMKSDYKWFQDYKPKLSKEVRDALSDIEWRLRRESLEFN |
| | KLSKSREQDARQLANWISSMCDVIGIANLVKKNNFFGGSGKREPGWD |
| | NFYKPKKENRWWINAIHKALTELSQNKGKRVILLPAMRTSITCPKCKY |
| | CDSKNRNGEKFNCLKCGIELNADIDVATENLATVAITAQSMPKPTCER |
| | SGDAKKPVRARKAKAPEFHDKLAPSYTVVLREAVKRPAATKKAGQA |
| | KKKKGSGSPAGSPTSTRSGGGSGTSSEVEFSHEYWMRHALTLAKRAR |
| | DEREVPVGAVLVLNNRVIGEGWNRAIGLHDPTAHAEIMALRQGGLVM |
| | QNYRLIDATLYVTFEPCVMCAGAMIHSRIGRVVFGVRNSKRGAAGSL |
| | MNVLNYPGMNHRVEITEGILADECAALLCDFYRMPRQVFNAQKKAQS |
| | SINSGGSSGGSSGSETPGTSESATPESSGGSSGGSSEVEFSHEYWMRHAL |
| | TLAKRAWDEREVPVGAVLVHNNRVIGEGWNRPIGRHDPTAHAEIMAL |
| | RQGGLVMQNYRLIDATLYVTLEPCVMCAGAMIHSRIGRVVFGARDAK |
| | TGAAGSLMDVLHHPGMNHRVEITEGILADECAALLSDFFRMRRQEIKA |
| | QKKAQSSTD (SEQ ID NO: 556) |
| dCasΦ.12 | MIKPTVSQFLTPGFKLIRNHSRTAGLKLKNEGEEACKKFVRENEIPKDE |
| (E567Q)- | CPNFQGGPAIANIIAKSREFTEWEIYQSSLAIQEVIFTLPKDKLPEPILKEE |
| XTEN10- | WRAQWLSEHGLDTVPYKEAAGLNLIIKNAVNTYKGVQVKVDNKNKN |
| ABE8e- | NLAKINRKNEIAKLNGEQEISFEEIKAFDDKGYLLQKPSPNKSIYCYQSV |
| TadA | SPKPFITSKYHNVNLPEEYIGYYRKSNEPIVSPYQFDRLRIPIGEPGYVPK |
| | WQYTFLSKKENKRRKLSKRIKNVSPILGIICIKKDWCVFDMRGLLRTNH |
| | WKKYHKPTDSINDLFDYFTGDPVIDTKANVVRFRYKMENGIVNYKPV |
| | REKKGKELLENICDQNGSCKLATVDVGQNNPVAIGLFELKKVNGELTK |
| | TLISRHPTPIDFCNKITAYRERYDKLESSIKLDAIKQLTSEQKIEVDNYNN |
| | NFTPQNTKQIVCSKLNINPNDLPWDKMISGTHFISEKAQVSNKSEIYFTS |
| | TDKGKTKDVMKSDYKWFQDYKPKLSKEVRDALSDIEWRLRRESLEFN |
| | KLSKSREQDARQLANWISSMCDVIGIQNLVKKNNFFGGSGKREPGWD |
| | NFYKPKKENRWWINAIHKALTELSQNKGKRVILLPAMRTSITCPKCKY |
| | CDSKNRNGEKFNCLKCGIELNADIDVATENLATVAITAQSMPKPTCER |
| | SGDAKKPVRARKAKAPEFHDKLAPSYTVVLREAVKRPAATKKAGQA |
| | KKKKGSGSPAGSPTSTRSGGGSGTSSEVEFSHEYWMRHALTLAKRAR |
| | DEREVPVGAVLVLNNRVIGEGWNRAIGLHDPTAHAEIMALRQGGLVM |
| | QNYRLIDATLYVTFEPCVMCAGAMIHSRIGRVVFGVRNSKRGAAGSL |
| | MNVLNYPGMNHRVEITEGILADECAALLCDFYRMPRQVFNAQKKAQS |
| | SINSGGSSGGSSGSETPGTSESATPESSGGSSGGSSEVEFSHEYWMRHAL |
| | TLAKRAWDEREVPVGAVLVHNNRVIGEGWNRPIGRHDPTAHAEIMAL |
| | RQGGLVMQNYRLIDATLYVTLEPCVMCAGAMIHSRIGRVVFGARDAK |
| | TGAAGSLMDVLHHPGMNHRVEITEGILADECAALLSDFFRMRRQEIKA |
| | QKKAQSSTD (SEQ ID NO: 557) |
| dCasΦ.12 | MIKPTVSQFLTPGFKLIRNHSRTAGLKLKNEGEEACKKFVRENEIPKDE |
| (D658A)- | CPNFQGGPAIANIIAKSREFTEWEIYQSSLAIQEVIFTLPKDKLPEPILKEE |
| XTEN10- | WRAQWLSEHGLDTVPYKEAAGLNLIIKNAVNTYKGVQVKVDNKNKN |
| ABE8e- | NLAKINRKNEIAKLNGEQEISFEEIKAFDDKGYLLQKPSPNKSIYCYQSV |
| TadA | SPKPFITSKYHNVNLPEEYIGYYRKSNEPIVSPYQFDRLRIPIGEPGYVPK |
| | WQYTFLSKKENKRRKLSKRIKNVSPILGIICIKKDWCVFDMRGLLRTNH |
| | WKKYHKPTDSINDLFDYFTGDPVIDTKANVVRFRYKMENGIVNYKPV |
| | REKKGKELLENICDQNGSCKLATVDVGQNNPVAIGLFELKKVNGELTK |
| | TLISRHPTPIDFCNKITAYRERYDKLESSIKLDAIKQLTSEQKIEVDNYNN |
| | NFTPQNTKQIVCSKLNINPNDLPWDKMISGTHFISEKAQVSNKSEIYFTS |
| | TDKGKTKDVMKSDYKWFQDYKPKLSKEVRDALSDIEWRLRRESLEFN |
| | KLSKSREQDARQLANWISSMCDVIGIENLVKKNNFFGGSGKREPGWD |
| | NFYKPKKENRWWINAIHKALTELSQNKGKRVILLPAMRTSITCPKCKY |
| | CDSKNRNGEKFNCLKCGIELNAAIDVATENLATVAITAQSMPKPTCER |
| | SGDAKKPVRARKAKAPEFHDKLAPSYTVVLREAVKRPAATKKAGQA |
| | KKKKGSGSPAGSPTSTRSGGGSGTSSEVEFSHEYWMRHALTLAKRAR |
| | DEREVPVGAVLVLNNRVIGEGWNRAIGLHDPTAHAEIMALRQGGLVM |
| | QNYRLIDATLYVTFEPCVMCAGAMIHSRIGRVVFGVRNSKRGAAGSL |
| | MNVLNYPGMNHRVEITEGILADECAALLCDFYRMPRQVFNAQKKAQS |
| | SINSGGSSGGSSGSETPGTSESATPESSGGSSGGSSEVEFSHEYWMRHAL |
| | TLAKRAWDEREVPVGAVLVHNNRVIGEGWNRPIGRHDPTAHAEIMAL |
| | RQGGLVMQNYRLIDATLYVTLEPCVMCAGAMIHSRIGRVVFGARDAK |
| | TGAAGSLMDVLHHPGMNHRVEITEGILADECAALLSDFFRMRRQEIKA |
| | QKKAQSSTD (SEQ ID NO: 558) |
| dCasΦ.12 | MIKPTVSQFLTPGFKLIRNHSRTAGLKLKNEGEEACKKFVRENEIPKDE |
| (D658N)- | CPNFQGGPAIANIIAKSREFTEWEIYQSSLAIQEVIFTLPKDKLPEPILKEE |
| XTEN10- | WRAQWLSEHGLDTVPYKEAAGLNLIIKNAVNTYKGVQVKVDNKNKN |
| ABE8e- | NLAKINRKNEIAKLNGEQEISFEEIKAFDDKGYLLQKPSPNKSIYCYQSV |
| TadA | SPKPFITSKYHNVNLPEEYIGYYRKSNEPIVSPYQFDRLRIPIGEPGYVPK |
| | WQYTFLSKKENKRRKLSKRIKNVSPILGIICIKKDWCVFDMRGLLRTNH |
| | WKKYHKPTDSINDLFDYFTGDPVIDTKANVVRFRYKMENGIVNYKPV |
| | REKKGKELLENICDQNGSCKLATVDVGQNNPVAIGLFELKKVNGELTK |
| | TLISRHPTPIDFCNKITAYRERYDKLESSIKLDAIKQLTSEQKIEVDNYNN |
| | NFTPQNTKQIVCSKLNINPNDLPWDKMISGTHFISEKAQVSNKSEIYFTS |
| | TDKGKTKDVMKSDYKWFQDYKPKLSKEVRDALSDIEWRLRRESLEFN |
| | KLSKSREQDARQLANWISSMCDVIGIENLVKKNNFFGGSGKREPGWD |
| | NFYKPKKENRWWINAIHKALTELSQNKGKRVILLPAMRTSITCPKCKY |
| | CDSKNRNGEKFNCLKCGIELNANIDVATENLATVAITAQSMPKPTCER |
| | SGDAKKPVRARKAKAPEFHDKLAPSYTVVLREAVKRPAATKKAGQA |
| | KKKKGSGSPAGSPTSTRSGGGSGTSSEVEFSHEYWMRHALTLAKRAR |
| | DEREVPVGAVLVLNNRVIGEGWNRAIGLHDPTAHAEIMALRQGGLVM |
| | QNYRLIDATLYVTFEPCVMCAGAMIHSRIGRVVFGVRNSKRGAAGSL |
| | MNVLNYPGMNHRVEITEGILADECAALLCDFYRMPRQVFNAQKKAQS |
| | SINSGGSSGGSSGSETPGTSESATPESSGGSSGGSSEVEFSHEYWMRHAL |
| | TLAKRAWDEREVPVGAVLVHNNRVIGEGWNRPIGRHDPTAHAEIMAL |
| | RQGGLVMQNYRLIDATLYVTLEPCVMCAGAMIHSRIGRVVFGARDAK |
| | TGAAGSLMDVLHHPGMNHRVEITEGILADECAALLSDFFRMRRQEIKA |
| | QKKAQSSTD (SEQ ID NO: 559) |

### Example 6: gRNA Optimization for dCasΦ.12(E567Q)-ABE8e Deaminase Fusion Protein

Base editing efficiency of the fusion protein was further explored by optimization of the gRNA design. An exemplary base editing fusion protein, dCasΦ.12(E567Q)-XTEN10-ABE8e (SEQ ID NO: 545), was selected based on the analysis conducted in Example 5. 72 gRNA designs were created, which targeted the same four sites as described in Example 5: FUT8-target 2, B2M-target 2, PDCD1-target 87, and PDCD1-target 75. Each guide was tested with a 36, 24, and 20 repeat length combined with 12, 14, 16, 18, 20, and 23 spacer lengths as shown in TABLE 7. Cells were treated and base modifications were analyzed according to the methods described in Example 1. Base editing levels were evaluated for each target site gRNA design.

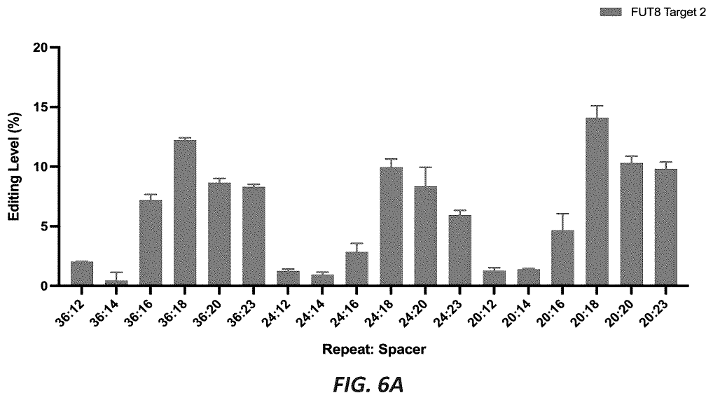

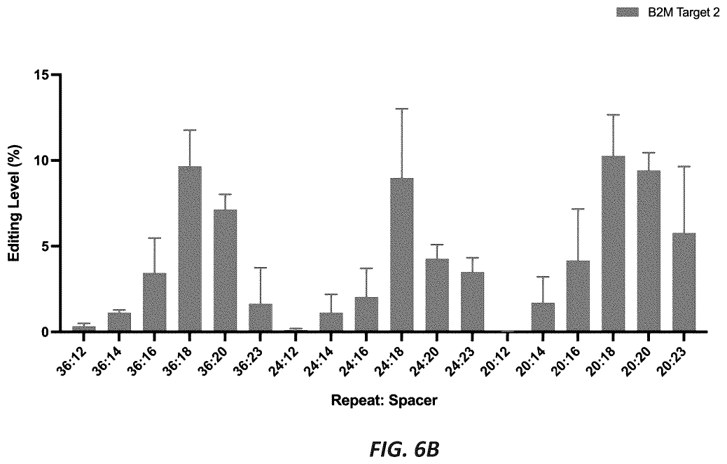

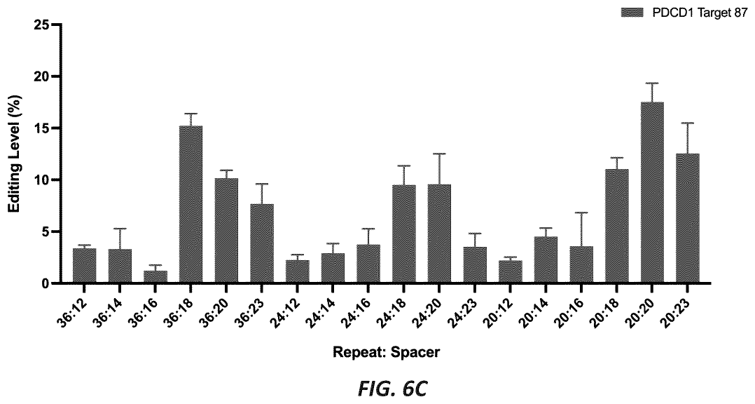

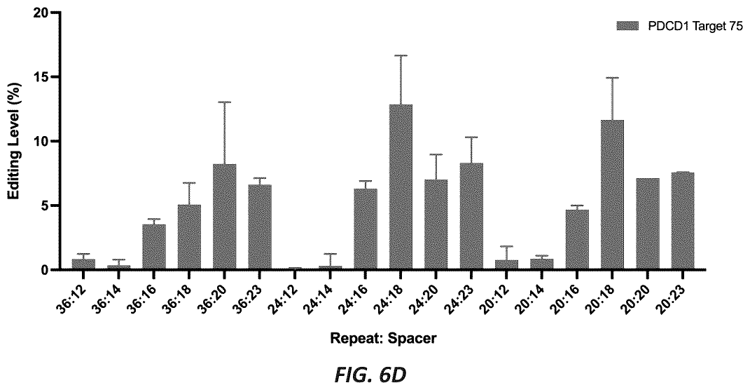

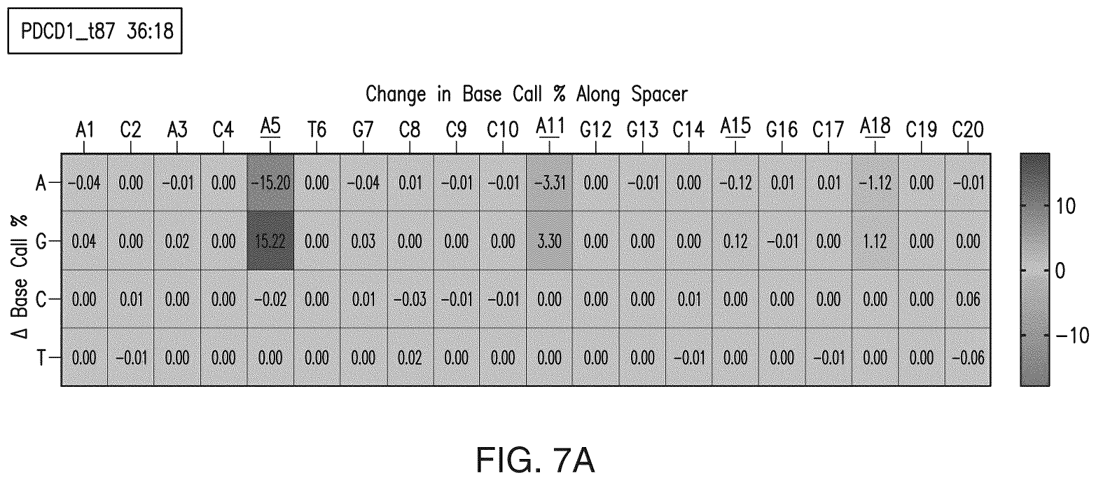

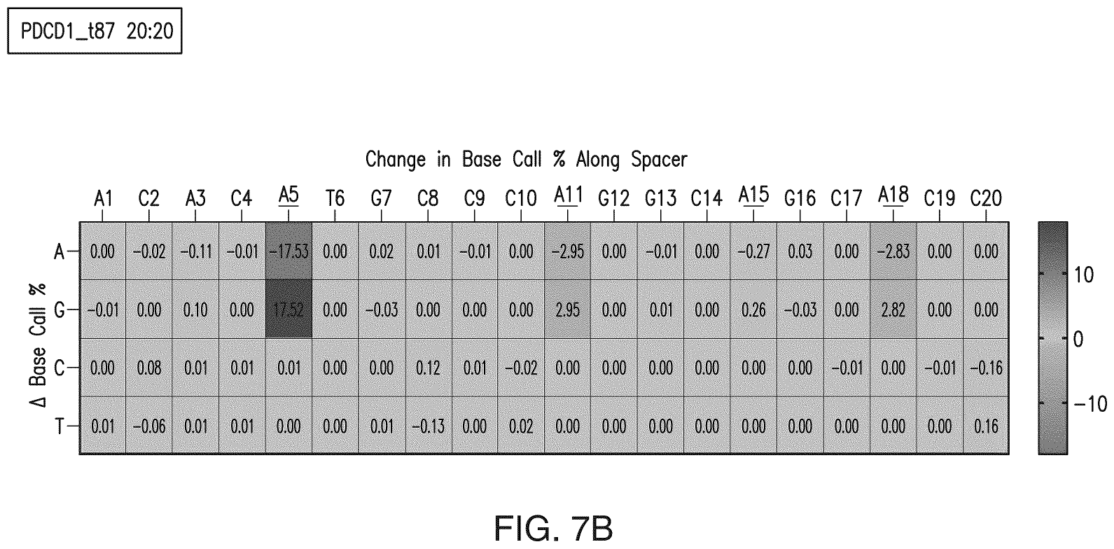

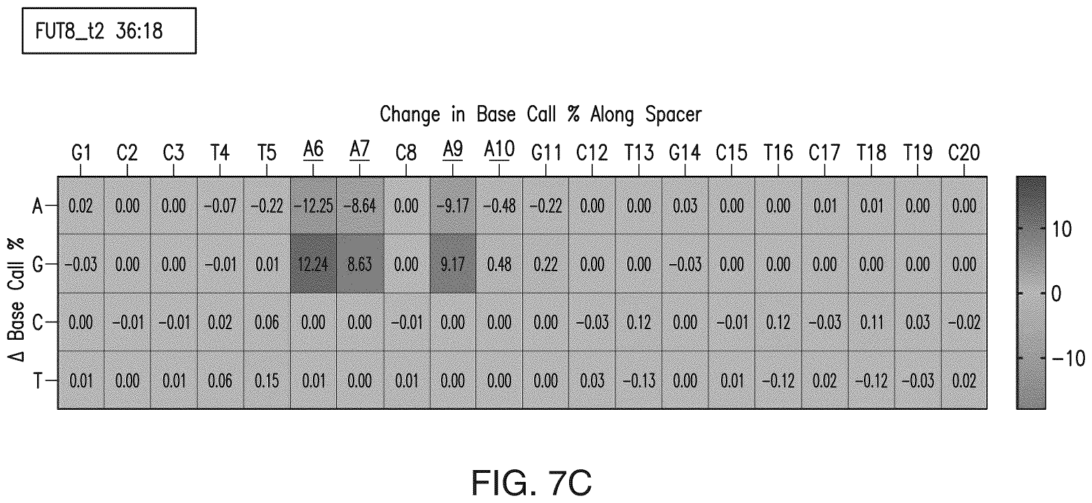

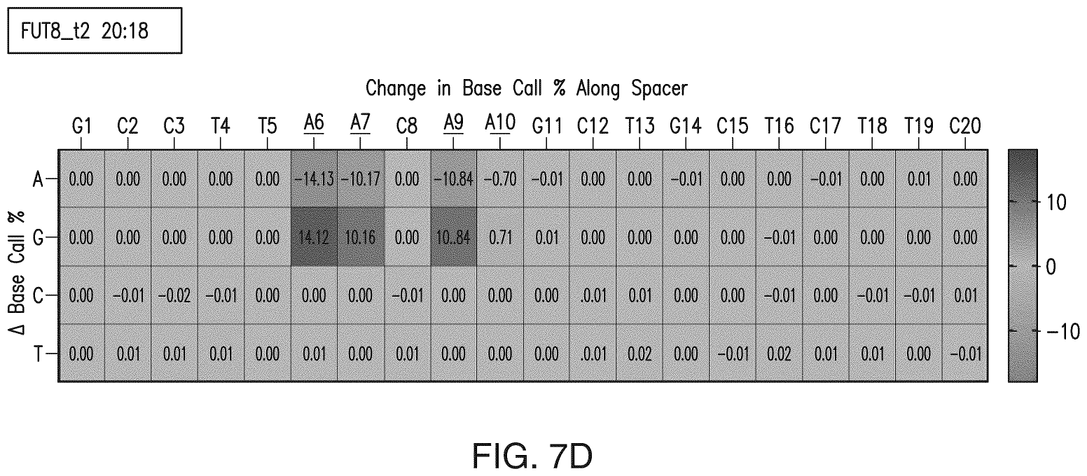

Exemplary results are shown in FIGS. 6A-6D. Optimized gRNA compositions for PDCD1-target 87 were observed to have markedly enhanced base editing function with repeat:spacer compositions comprising (36:18) or (20:20) as shown in FIG. 7A and FIG. 7B, respectively. Optimized gRNA compositions for FUT8-target 2 were observed to have markedly enhanced base editing function with repeat:spacer compositions comprising (36:18) or (20:18) as shown in FIG. 7C and FIG. 7D.

TABLE 7
Guide RNA Compositions (Repeats and Spacers)

| gRNA | | | | |
|---|---|---|---|---|
| Composition | | | | |
| and Target | | | | |
| (Repeat | | | | |
| residues: | | | | |
| Spacer | Repeat (DNA | Repeat (RNA | Spacer (DNA | Spacer (RNA |
| residues) | Sequence) | Sequence) | Sequence) | Sequence) |
| FUT8-target | CTTTCAAGAC | CUUUCAAGAC | GCCTTAACAA | GCCUUAACA |
| 2 (36:12) | TAATAGATTG | UAAUAGAUUG | GC (SEQ ID | AGC (SEQ ID |
| | CTCCTTACGA | CUCCUUACGA | NO: 653) | NO: 786) |
| | GGAGAC (SEQ | GGAGAC (SEQ | | |
| | ID NO: 650) | ID NO: 783) | | |
| FUT8-target | CTTTCAAGAC | CUUUCAAGAC | GCCTTAACAA | GCCUUAACA |
| 2 (36:14) | TAATAGATTG | UAAUAGAUUG | GCTG (SEQ ID | AGCUG (SEQ |
| | CTCCTTACGA | CUCCUUACGA | NO: 654) | ID NO: 787) |
| | GGAGAC (SEQ | GGAGAC (SEQ | | |
| | ID NO: 650) | ID NO: 783) | | |
| FUT8-target | CTTTCAAGAC | CUUUCAAGAC | GCCTTAACAA | GCCUUAACA |
| 2 (36:16) | TAATAGATTG | UAAUAGAUUG | GCTGCT (SEQ | AGCUGCU |
| | CTCCTTACGA | CUCCUUACGA | ID NO: 655) | (SEQ ID NO: |
| | GGAGAC (SEQ | GGAGAC (SEQ | | 788) |
| | ID NO: 650) | ID NO: 783) | | |
| FUT8-target | CTTTCAAGAC | CUUUCAAGAC | GCCTTAACAA | GCCUUAACA |
| 2 (36:18) | TAATAGATTG | UAAUAGAUUG | GCTGCTCT | AGCUGCUCU |
| | CTCCTTACGA | CUCCUUACGA | (SEQ ID NO: | (SEQ ID NO: |
| | GGAGAC (SEQ | GGAGAC (SEQ | 656) | 789) |
| | ID NO: 650) | ID NO: 783) | | |
| FUT8-target | CTTTCAAGAC | CUUUCAAGAC | GCCTTAACAA | GCCUUAACA |
| 2 (36:20) | TAATAGATTG | UAAUAGAUUG | GCTGCTCTTC | AGCUGCUCU |
| | CTCCTTACGA | CUCCUUACGA | (SEQ ID NO: | UC (SEQ ID |
| | GGAGAC (SEQ | GGAGAC (SEQ | 657) | NO: 790) |
| | ID NO: 650) | ID NO: 783) | | |
| FUT8-target | CTTTCAAGAC | CUUUCAAGAC | GCCTTAACAA | GCCUUAACA |
| 2 (36:23) | TAATAGATTG | UAAUAGAUUG | GCTGCTCTTCT | AGCUGCUCU |
| | CTCCTTACGA | CUCCUUACGA | | |
| | GGAGAC (SEQ | GGAGAC (SEQ | AA (SEQ ID | UCUAA (SEQ |
| | ID NO: 650) | ID NO: 783) | NO: 658) | ID NO: 791) |
| B2M-target 2 | CTTTCAAGAC | CUUUCAAGAC | GGGCCGAGAT | GGGCCGAGA |
| (36:12) | TAATAGATTG | UAAUAGAUUG | GT (SEQ ID | UGU (SEQ ID |
| | CTCCTTACGA | CUCCUUACGA | NO: 659) | NO: 792) |
| | GGAGAC (SEQ | GGAGAC (SEQ | | |
| | ID NO: 650) | ID NO: 783) | | |
| B2M-target 2 | CTTTCAAGAC | CUUUCAAGAC | GGGCCGAGAT | GGGCCGAGA |
| (36:14) | TAATAGATTG | UAAUAGAUUG | GTCT (SEQ ID | UGUCU (SEQ |
| | CTCCTTACGA | CUCCUUACGA | NO: 660) | ID NO: 793) |
| | GGAGAC (SEQ | GGAGAC (SEQ | | |
| | ID NO: 650) | ID NO: 783) | | |
| B2M-target 2 | CTTTCAAGAC | CUUUCAAGAC | GGGCCGAGAT | GGGCCGAGA |
| (36:16) | TAATAGATTG | UAAUAGAUUG | GTCTCG (SEQ | UGUCUCG |
| | CTCCTTACGA | CUCCUUACGA | ID NO: 661) | (SEQ ID NO: |
| | GGAGAC (SEQ | GGAGAC (SEQ | | 794) |
| | ID NO: 650) | ID NO: 783) | | |
| B2M-target 2 | CTTTCAAGAC | CUUUCAAGAC | GGGCCGAGAT | GGGCCGAGA |
| (36:18) | TAATAGATTG | UAAUAGAUUG | GTCTCGCT | UGUCUCGCU |
| | CTCCTTACGA | CUCCUUACGA | (SEQ ID NO: | (SEQ ID NO: |
| | GGAGAC (SEQ | GGAGAC (SEQ | 662) | 795) |
| | ID NO: 650) | ID NO: 783) | | |
| B2M-target 2 | CTTTCAAGAC | CUUUCAAGAC | ATATAAGTGG | AUAUAAGUG |
| (36:20) | TAATAGATTG | UAAUAGAUUG | AGGCGTCGCG | GAGGCGUCG |
| | CTCCTTACGA | CUCCUUACGA | (SEQ ID NO: | CG |
| | GGAGAC (SEQ | GGAGAC (SEQ | 663) | (SEQ ID NO: |
| | ID NO: 650) | ID NO: 783) | | 796) |
| B2M-target 2 | CTTTCAAGAC | CUUUCAAGAC | GGGCCGAGAT | GGGCCGAGA |
| (36:23) | TAATAGATTG | UAAUAGAUUG | GTCTCGCTCCG | UGUCUCGCU |
| | CTCCTTACGA | CUCCUUACGA | TG (SEQ ID | CCGUG (SEQ |
| | GGAGAC (SEQ | GGAGAC (SEQ | NO: 664) | ID NO: 797) |
| | ID NO: 650) | ID NO: 783) | | |
| PDCD1- | CTTTCAAGAC | CUUUCAAGAC | ACACATGCCC | ACACAUGCC |
| target 87 | TAATAGATTG | UAAUAGAUUG | AG (SEQ ID | CAG (SEQ ID |
| (36:12) | CTCCTTACGA | CUCCUUACGA | NO: 665) | NO: 798) |
| | GGAGAC (SEQ | GGAGAC (SEQ | | |
| | ID NO: 650) | ID NO: 783) | | |
| PDCD1- | CTTTCAAGAC | CUUUCAAGAC | ACACATGCCC | ACACAUGCC |
| target 87 | TAATAGATTG | UAAUAGAUUG | AGGC (SEQ ID | CAGGC (SEQ |
| (36:14) | CTCCTTACGA | CUCCUUACGA | NO: 666) | ID NO: 799) |
| | GGAGAC (SEQ | GGAGAC (SEQ | | |
| | ID NO: 650) | ID NO: 783) | | |
| PDCD1- | CTTTCAAGAC | CUUUCAAGAC | ACACATGCCC | ACACAUGCC |
| target 87 | TAATAGATTG | UAAUAGAUUG | AGGCAG (SEQ | CAGGCAG |
| (36:16) | CTCCTTACGA | CUCCUUACGA | ID NO: 667) | (SEQ ID NO: |
| | GGAGAC (SEQ | GGAGAC (SEQ | | 800) |
| | ID NO: 650) | ID NO: 783) | | |
| PDCD1- | CTTTCAAGAC | CUUUCAAGAC | ACACATGCCC | ACACAUGCC |
| target 87 | TAATAGATTG | UAAUAGAUUG | AGGCAGCA | CAGGCAGCA |
| (36:18) | CTCCTTACGA | CUCCUUACGA | (SEQ ID NO: | (SEQ ID NO: |
| | GGAGAC (SEQ | GGAGAC (SEQ | 668) | 801) |
| | ID NO: 650) | ID NO: 783) | | |
| PDCD1- | CTTTCAAGAC | CUUUCAAGAC | ACACATGCCC | ACACAUGCC |
| target 87 | TAATAGATTG | UAAUAGAUUG | AGGCAGCACC | CAGGCAGCA |
| (36:20) | CTCCTTACGA | CUCCUUACGA | (SEQ ID NO: | CC (SEQ ID |
| | GGAGAC (SEQ | GGAGAC (SEQ | 669) | NO: 802) |
| | ID NO: 650) | ID NO: 783) | | |
| PDCD1- | CTTTCAAGAC | CUUUCAAGAC | ACACATGCCC | ACACAUGCC |
| target 87 | TAATAGATTG | UAAUAGAUUG | AGGCAGCACC | CAGGCAGCA |
| (36:23) | CTCCTTACGA | CUCCUUACGA | TCA (SEQ ID | CCUCA (SEQ |
| | GGAGAC (SEQ | GGAGAC (SEQ | NO: 670) | ID NO: 803) |
| | ID NO: 650) | ID NO: 783) | | |
| PDCD1- | CTTTCAAGAC | CUUUCAAGAC | GAGCAGCCAA | GAGCAGCCA |
| target 87 | TAATAGATTG | UAAUAGAUUG | GG | AGG |
| (36:12) | CTCCTTACGA | CUCCUUACGA | (SEQ ID NO: | (SEQ ID NO: |
| | GGAGAC (SEQ | GGAGAC (SEQ | 671) | 804) |
| | ID NO: 650) | ID NO: 783) | | |
| PDCD1- | CTTTCAAGAC | CUUUCAAGAC | GAGCAGCCAA | GAGCAGCCA |
| target 75 | TAATAGATTG | UAAUAGAUUG | GGTG (SEQ ID | AGGUG (SEQ |
| (36:14) | CTCCTTACGA | CUCCUUACGA | NO: 672) | ID NO: 805) |
| | GGAGAC (SEQ | GGAGAC (SEQ | | |
| | ID NO: 650) | ID NO: 783) | | |
| PDCD1- | CTTTCAAGAC | CUUUCAAGAC | GAGCAGCCAA | GAGCAGCCA |
| target 75 | TAATAGATTG | UAAUAGAUUG | GGTGCC (SEQ | AGGUGCC |
| (36:16) | CTCCTTACGA | CUCCUUACGA | ID NO: 673) | (SEQ ID NO: |
| | GGAGAC (SEQ | GGAGAC (SEQ | | 806) |
| | ID NO: 650) | ID NO: 783) | | |
| PDCD1- | CTTTCAAGAC | CUUUCAAGAC | GAGCAGCCAA | GAGCAGCCA |
| target 75 | TAATAGATTG | UAAUAGAUUG | GGTGCCCC | AGGUGCCCC |
| (36:18) | CTCCTTACGA | CUCCUUACGA | (SEQ ID NO: | (SEQ ID NO: |
| | GGAGAC (SEQ | GGAGAC (SEQ | 674) | 807) |
| | ID NO: 650) | ID NO: 783) | | |
| PDCD1- | CTTTCAAGAC | CUUUCAAGAC | GAGCAGCCAA | GAGCAGCCA |
| target 75 | TAATAGATTG | UAAUAGAUUG | GGTGCCCCTG | AGGUGCCCC |
| (36:20) | CTCCTTACGA | CUCCUUACGA | (SEQ ID NO: | UG (SEQ ID |
| | GGAGAC (SEQ | GGAGAC (SEQ | 675) | NO: 808) |
| | ID NO: 650) | ID NO: 783) | | |
| PDCD1- | CTTTCAAGAC | CUUUCAAGAC | GAGCAGCCAA | GAGCAGCCA |
| target 75 | TAATAGATTG | UAAUAGAUUG | GGTGCCCCTG | AGGUGCCCC |
| (36:23) | CTCCTTACGA | CUCCUUACGA | GCA (SEQ ID | UGGCA (SEQ |
| | GGAGAC (SEQ | GGAGAC (SEQ | NO: 676) | ID NO: 809) |
| | ID NO: 650) | ID NO: 783) | | |
| FUT8-target | ATTGCTCCTT | AUUGCUCCUU | GCCTTAACAA | GCCUUAACA |
| 2 (20:12) | ACGAGGAGA | ACGAGGAGAC | GC (SEQ ID | AGC (SEQ ID |
| | C (SEQ ID NO: | (SEQ ID NO: | NO: 653) | NO: 786) |
| | 651) | 784) | | |
| FUT8-target | ATTGCTCCTT | AUUGCUCCUU | GCCTTAACAA | GCCUUAACA |
| 2 (20:14) | ACGAGGAGA | ACGAGGAGAC | GCTG (SEQ ID | AGCUG (SEQ |
| | C (SEQ ID NO: | (SEQ ID NO: | NO: 654) | ID NO: 787) |
| | 651) | 784) | | |
| FUT8-target | ATTGCTCCTT | AUUGCUCCUU | GCCTTAACAA | GCCUUAACA |
| 2 (20:16) | ACGAGGAGA | ACGAGGAGAC | GCTGCT (SEQ | AGCUGCU |
| | C (SEQ ID NO: | (SEQ ID NO: | ID NO: 655) | (SEQ ID NO: |
| | 651) | 784) | | 788) |
| FUT8-target | ATTGCTCCTT | AUUGCUCCUU | GCCTTAACAA | GCCUUAACA |
| 2 (20:18) | ACGAGGAGA | ACGAGGAGAC | GCTGCTCT | AGCUGCUCU |
| | C (SEQ ID NO: | (SEQ ID NO: | (SEQ ID NO: | (SEQ ID NO: |
| | 651) | 784) | 656) | 789) |
| FUT8-target | ATTGCTCCTT | AUUGCUCCUU | GCCTTAACAA | GCCUUAACA |
| 2 (20:20) | ACGAGGAGA | ACGAGGAGAC | GCTGCTCTTC | AGCUGCUCU |
| | C (SEQ ID NO: | (SEQ ID NO: | (SEQ ID NO: | UC (SEQ ID |
| | 651) | 784) | 657) | NO: 790) |
| FUT8-target | ATTGCTCCTT | AUUGCUCCUU | GCCTTAACAA | GCCUUAACA |
| 2 (20:23) | ACGAGGAGA | ACGAGGAGAC | GCTGCTCTTCT | AGCUGCUCU |
| | C (SEQ ID NO: | (SEQ ID NO: | AA (SEQ ID | UCUAA (SEQ |
| | 651) | 784) | NO: 658) | ID NO: 791) |
| B2M-target 2 | ATTGCTCCTT | AUUGCUCCUU | GGGCCGAGAT | GGGCCGAGA |
| (20:12) | ACGAGGAGA | ACGAGGAGAC | GT (SEQ ID | UGU (SEQ ID |
| | C (SEQ ID NO: | (SEQ ID NO: | NO: 659) | NO: 792) |
| | 651) | 784) | | |
| B2M-target 2 | ATTGCTCCTT | AUUGCUCCUU | GGGCCGAGAT | GGGCCGAGA |
| (20:14) | ACGAGGAGA | ACGAGGAGAC | GTCT (SEQ ID | UGUCU (SEQ |
| | C (SEQ ID NO: | (SEQ ID NO: | NO: 660) | ID NO: 793) |
| | 651) | 784) | | |
| B2M-target 2 | ATTGCTCCTT | AUUGCUCCUU | GGGCCGAGAT | GGGCCGAGA |
| (20:16) | ACGAGGAGA | ACGAGGAGAC | GTCTCG (SEQ | UGUCUCG |
| | C (SEQ ID NO: | (SEQ ID NO: | ID NO: 661) | (SEQ ID NO: |
| | 651) | 784) | | 794) |
| B2M-target 2 | ATTGCTCCTT | AUUGCUCCUU | GGGCCGAGAT | GGGCCGAGA |
| (20:18) | ACGAGGAGA | ACGAGGAGAC | GTCTCGCT | UGUCUCGCU |
| | C (SEQ ID NO: | (SEQ ID NO: | (SEQ ID NO: | (SEQ ID NO: |
| | 651) | 784) | 662) | 795) |
| B2M-target 2 | ATTGCTCCTT | AUUGCUCCUU | ATATAAGTGG | AUAUAAGUG |
| (20:20) | ACGAGGAGA | ACGAGGAGAC | AGGCGTCGCG | GAGGCGUCG |
| | C (SEQ ID NO: | (SEQ ID NO: | (SEQ ID NO: | CG |
| | 651) | 784) | 663) | (SEQ ID NO: |
| | | | | 796) |
| B2M-target 2 | ATTGCTCCTT | AUUGCUCCUU | GGGCCGAGAT | GGGCCGAGA |
| (20:23) | ACGAGGAGA | ACGAGGAGAC | GTCTCGCTCCG | UGUCUCGCU |
| | C (SEQ ID NO: | (SEQ ID NO: | TG (SEQ ID | CCGUG (SEQ |
| | 651) | 784) | NO: 664) | ID NO: 797) |
| PDCD1- | ATTGCTCCTT | AUUGCUCCUU | ACACATGCCC | ACACAUGCC |
| target 87 | ACGAGGAGA | ACGAGGAGAC | AG (SEQ ID | CAG (SEQ ID |
| (20:12) | C (SEQ ID NO: | (SEQ ID NO: | NO: 665) | NO: 798) |
| | 651) | 784) | | |
| PDCD1- | ATTGCTCCTT | AUUGCUCCUU | ACACATGCCC | ACACAUGCC |
| target 87 | ACGAGGAGA | ACGAGGAGAC | AGGC (SEQ ID | CAGGC (SEQ |
| (20:14) | C (SEQ ID NO: | (SEQ ID NO: | NO: 666) | ID NO: 799) |
| | 651) | 784) | | |
| PDCD1- | ATTGCTCCTT | AUUGCUCCUU | ACACATGCCC | ACACAUGCC |
| target 87 | ACGAGGAGA | ACGAGGAGAC | AGGCAG (SEQ | CAGGCAG |
| (20:16) | C (SEQ ID NO: | (SEQ ID NO: | ID NO: 667) | (SEQ ID NO: |
| | 651) | 784) | | 800) |
| PDCD1- | ATTGCTCCTT | AUUGCUCCUU | ACACATGCCC | ACACAUGCC |
| target 87 | ACGAGGAGA | ACGAGGAGAC | AGGCAGCA | CAGGCAGCA |
| (20:18) | C (SEQ ID NO: | (SEQ ID NO: | (SEQ ID NO: | (SEQ ID NO: |
| | 651) | 784) | 668) | 801) |
| PDCD1- | ATTGCTCCTT | AUUGCUCCUU | ACACATGCCC | ACACAUGCC |
| target 87 | ACGAGGAGA | ACGAGGAGAC | AGGCAGCACC | CAGGCAGCA |
| (20:20) | C (SEQ ID NO: | (SEQ ID NO: | (SEQ ID NO: | CC (SEQ ID |
| | 651) | 784) | 669) | NO: 802) |
| PDCD1- | ATTGCTCCTT | AUUGCUCCUU | ACACATGCCC | ACACAUGCC |
| target 87 | ACGAGGAGA | ACGAGGAGAC | AGGCAGCACC | CAGGCAGCA |
| (20:23) | C (SEQ ID NO: | (SEQ ID NO: | TCA (SEQ ID | CCUCA (SEQ |
| | 651) | 784) | NO: 670) | ID NO: 803) |
| PDCD1- | ATTGCTCCTT | AUUGCUCCUU | GAGCAGCCAA | GAGCAGCCA |
| target 75 | ACGAGGAGA | ACGAGGAGAC | GG (SEQ ID | AGG |
| (20:12) | C (SEQ ID NO: | (SEQ ID NO: | NO: 671) | (SEQ ID NO: |
| | 651) | 784) | | 804) |
| PDCD1- | ATTGCTCCTT | AUUGCUCCUU | GAGCAGCCAA | GAGCAGCCA |
| target 75 | ACGAGGAGA | ACGAGGAGAC | GGTG (SEQ ID | AGGUG (SEQ |
| (20:14) | C (SEQ ID NO: | (SEQ ID NO: | NO: 672) | ID NO: 805) |
| | 651) | 784) | | |
| PDCD1- | ATTGCTCCTT | AUUGCUCCUU | GAGCAGCCAA | GAGCAGCCA |
| target 75 | ACGAGGAGA | ACGAGGAGAC | GGTGCC (SEQ | AGGUGCC |
| (20:16) | C (SEQ ID NO: | (SEQ ID NO: | ID NO: 673) | (SEQ ID NO: |
| | 651) | 784) | | 806) |
| PDCD1- | ATTGCTCCTT | AUUGCUCCUU | GAGCAGCCAA | GAGCAGCCA |
| target 75 | ACGAGGAGA | ACGAGGAGAC | GGTGCCCC | AGGUGCCCC |
| (20:18) | C (SEQ ID NO: | (SEQ ID NO: | (SEQ ID NO: | (SEQ ID NO: |
| | 651) | 784) | 674) | 807) |
| PDCD1- | ATTGCTCCTT | AUUGCUCCUU | GAGCAGCCAA | GAGCAGCCA |
| target 75 | ACGAGGAGA | ACGAGGAGAC | GGTGCCCCTG | AGGUGCCCC |
| (20:20) | C (SEQ ID NO: | (SEQ ID NO: | (SEQ ID NO: | UG (SEQ ID |
| | 651) | 784) | 675) | NO: 808) |
| PDCD1- | ATTGCTCCTT | AUUGCUCCUU | GAGCAGCCAA | GAGCAGCCA |
| target 75 | ACGAGGAGA | ACGAGGAGAC | GGTGCCCCTG | AGGUGCCCC |
| (20:23) | C (SEQ ID NO: | (SEQ ID NO: | GCA (SEQ ID | UGGCA (SEQ |
| | 651) | 784) | NO: 676) | ID NO: 809) |
| FUT8-target | ATAGATTGCT | AUAGAUUGCU | GCCTTAACAA | GCCUUAACA |
| 2 (24:12) | CCTTACGAGG | CCUUACGAGG | GC (SEQ ID | AGC (SEQ ID |
| | AGAC (SEQ ID | AGAC (SEQ ID | NO: 653) | NO: 786) |
| | NO: 652) | NO: 785) | | |
| FUT8-target | ATAGATTGCT | AUAGAUUGCU | GCCTTAACAA | GCCUUAACA |
| 2 (24:14) | CCTTACGAGG | CCUUACGAGG | GCTG (SEQ ID | AGCUG (SEQ |
| | AGAC (SEQ ID | AGAC (SEQ ID | NO: 654) | ID NO: 787) |
| | NO: 652) | NO: 785) | | |
| FUT8-target | ATAGATTGCT | AUAGAUUGCU | GCCTTAACAA | GCCUUAACA |
| 2 (24:16) | CCTTACGAGG | CCUUACGAGG | GCTGCT (SEQ | AGCUGCU |
| | AGAC (SEQ ID | AGAC (SEQ ID | ID NO: 655) | (SEQ ID NO: |
| | NO: 652) | NO: 785) | | 788) |
| FUT8-target | ATAGATTGCT | AUAGAUUGCU | GCCTTAACAA | GCCUUAACA |
| 2 (24:18) | CCTTACGAGG | CCUUACGAGG | GCTGCTCT | AGCUGCUCU |
| | AGAC (SEQ ID | AGAC (SEQ ID | (SEQ ID NO: | (SEQ ID NO: |
| | NO: 652) | NO: 785) | 656) | 789) |
| FUT8-target | ATAGATTGCT | AUAGAUUGCU | GCCTTAACAA | GCCUUAACA |
| 2 (24:20) | CCTTACGAGG | CCUUACGAGG | GCTGCTCTTC | AGCUGCUCU |
| | AGAC (SEQ ID | AGAC (SEQ ID | (SEQ ID NO: | UC (SEQ ID |
| | NO: 652) | NO: 785) | 657) | NO: 790) |
| FUT8-target | ATAGATTGCT | AUAGAUUGCU | GCCTTAACAA | GCCUUAACA |
| 2 (24:23) | CCTTACGAGG | CCUUACGAGG | GCTGCTCTTCT | AGCUGCUCU |
| | AGAC (SEQ ID | AGAC (SEQ ID | AA (SEQ ID | UCUAA (SEQ |
| | NO: 652) | NO: 785) | NO: 658) | ID NO: 791) |
| B2M-target 2 | ATAGATTGCT | AUAGAUUGCU | GGGCCGAGAT | GGGCCGAGA |
| (24:12) | CCTTACGAGG | CCUUACGAGG | GT (SEQ ID | UGU (SEQ ID |
| | AGAC (SEQ ID | AGAC (SEQ ID | NO: 659) | NO: 792) |
| | NO: 652) | NO: 785) | | |
| B2M-target 2 | ATAGATTGCT | AUAGAUUGCU | GGGCCGAGAT | GGGCCGAGA |
| (24:14) | CCTTACGAGG | CCUUACGAGG | GTCT (SEQ ID | UGUCU (SEQ |
| | AGAC (SEQ ID | AGAC (SEQ ID | NO: 660) | ID NO: 793) |
| | NO: 652) | NO: 785) | | |
| B2M-target 2 | ATAGATTGCT | AUAGAUUGCU | GGGCCGAGAT | GGGCCGAGA |
| (24:16) | CCTTACGAGG | CCUUACGAGG | GTCTCG (SEQ | UGUCUCG |
| | AGAC (SEQ ID | AGAC (SEQ ID | ID NO: 661) | (SEQ ID NO: |
| | NO: 652) | NO: 785) | | 794) |
| B2M-target 2 | ATAGATTGCT | AUAGAUUGCU | GGGCCGAGAT | GGGCCGAGA |
| (24:18) | CCTTACGAGG | CCUUACGAGG | GTCTCGCT | UGUCUCGCU |
| | AGAC (SEQ ID | AGAC (SEQ ID | (SEQ ID NO: | (SEQ ID NO: |
| | NO: 652) | NO: 785) | 662) | 795) |
| B2M-target 2 | ATAGATTGCT | AUAGAUUGCU | ATATAAGTGG | AUAUAAGUG |
| (24:20) | CCTTACGAGG | CCUUACGAGG | AGGCGTCGCG | GAGGCGUCG |
| | AGAC (SEQ ID | AGAC (SEQ ID | (SEQ ID NO: | CG (SEQ ID |
| | NO: 652) | NO: 785) | 663) | NO: 796) |
| B2M-target 2 | ATAGATTGCT | AUAGAUUGCU | GGGCCGAGAT | GGGCCGAGA |
| (24:23) | CCTTACGAGG | CCUUACGAGG | GTCTCGCTCCG | UGUCUCGCU |
| | AGAC (SEQ ID | AGAC (SEQ ID | TG (SEQ ID | CCGUG (SEQ |
| | NO: 652) | NO: 785) | NO: 664) | ID NO: 797) |
| PDCD1- | ATAGATTGCT | AUAGAUUGCU | ACACATGCCC | ACACAUGCC |
| target 87 | CCTTACGAGG | CCUUACGAGG | AG (SEQ ID | CAG (SEQ ID |
| (24:12) | AGAC (SEQ ID | AGAC (SEQ ID | NO: 665) | NO: 798) |
| | NO: 652) | NO: 785) | | |
| PDCD1- | ATAGATTGCT | AUAGAUUGCU | ACACATGCCC | ACACAUGCC |
| target 87 | CCTTACGAGG | CCUUACGAGG | AGGC (SEQ ID | CAGGC (SEQ |
| (24:14) | AGAC (SEQ ID | AGAC (SEQ ID | NO: 666) | ID NO: 799) |
| | NO: 652) | NO: 785) | | |
| PDCD1- | ATAGATTGCT | AUAGAUUGCU | ACACATGCCC | ACACAUGCC |
| target 87 | CCTTACGAGG | CCUUACGAGG | AGGCAG (SEQ | CAGGCAG |
| (24:16) | AGAC (SEQ ID | AGAC (SEQ ID | ID NO: 667) | (SEQ ID NO: |
| | NO: 652) | NO: 785) | | 800) |
| PDCD1- | ATAGATTGCT | AUAGAUUGCU | ACACATGCCC | ACACAUGCC |
| target 87 | CCTTACGAGG | CCUUACGAGG | AGGCAGCA | CAGGCAGCA |
| (24:18) | AGAC (SEQ ID | AGAC (SEQ ID | (SEQ ID NO: | (SEQ ID NO: |
| | NO: 652) | NO: 785) | 668) | 801) |
| PDCD1- | ATAGATTGCT | AUAGAUUGCU | ACACATGCCC | ACACAUGCC |
| target 87 | CCTTACGAGG | CCUUACGAGG | AGGCAGCACC | CAGGCAGCA |
| (24:20) | AGAC (SEQ ID | AGAC (SEQ ID | (SEQ ID NO: | CC (SEQ ID |
| | NO: 652) | NO: 785) | 669) | NO: 802) |
| PDCD1- | ATAGATTGCT | AUAGAUUGCU | ACACATGCCC | ACACAUGCC |
| target 87 | CCTTACGAGG | CCUUACGAGG | AGGCAGCACC | CAGGCAGCA |
| (24:23) | AGAC (SEQ ID | AGAC (SEQ ID | TCA (SEQ ID | CCUCA (SEQ |
| | NO: 652) | NO: 785) | NO: 670) | ID NO: 803) |
| PDCD1- | ATAGATTGCT | AUAGAUUGCU | GAGCAGCCAA | GAGCAGCCA |
| target 75 | CCTTACGAGG | CCUUACGAGG | GG | AGG |
| (24:12) | AGAC (SEQ ID | AGAC (SEQ ID | (SEQ ID NO: | (SEQ ID NO: |
| | NO: 652) | NO: 785) | 671) | 804) |
| PDCD1- | ATAGATTGCT | AUAGAUUGCU | GAGCAGCCAA | GAGCAGCCA |
| target 75 | CCTTACGAGG | CCUUACGAGG | GGTG | AGGUG (SEQ |
| (24:14) | AGAC (SEQ ID | AGAC (SEQ ID | (SEQ ID NO: | ID NO: 805) |
| | NO: 652) | NO: 785) | 672) | |
| PDCD1- | ATAGATTGCT | AUAGAUUGCU | GAGCAGCCAA | GAGCAGCCA |
| target 75 | CCTTACGAGG | CCUUACGAGG | GGTGCC | AGGUGCC |
| (24:16) | AGAC (SEQ ID | AGAC (SEQ ID | (SEQ ID NO: | (SEQ ID NO: |
| | NO: 652) | NO: 785) | 673) | 806) |
| PDCD1- | ATAGATTGCT | AUAGAUUGCU | GAGCAGCCAA | GAGCAGCCA |
| target 75 | CCTTACGAGG | CCUUACGAGG | GGTGCCCC | AGGUGCCCC |
| (24:18) | AGAC (SEQ ID | AGAC (SEQ ID | (SEQ ID NO: | (SEQ ID NO: |
| | NO: 652) | NO: 785) | 674) | 807) |
| PDCD1- | ATAGATTGCT | AUAGAUUGCU | GAGCAGCCAA | GAGCAGCCA |
| target 75 | CCTTACGAGG | CCUUACGAGG | GGTGCCCCTG | AGGUGCCCC |
| (24:20) | AGAC (SEQ ID | AGAC (SEQ ID | (SEQ ID NO: | UG (SEQ ID |
| | NO: 652) | NO: 785) | 675) | NO: 808) |
| PDCD1- | ATAGATTGCT | AUAGAUUGCU | GAGCAGCCAA | GAGCAGCCA |
| target 75 | CCTTACGAGG | CCUUACGAGG | GGTGCCCCTG | AGGUGCCCC |
| (24:23) | AGAC (SEQ ID | AGAC (SEQ ID | GCA (SEQ ID | UGGCA (SEQ |
| | NO: 652) | NO: 785) | NO: 676) | ID NO: 809) |

### Example 7: Activation of Gene Expression with CasLambda Fusion (CRISPRa)

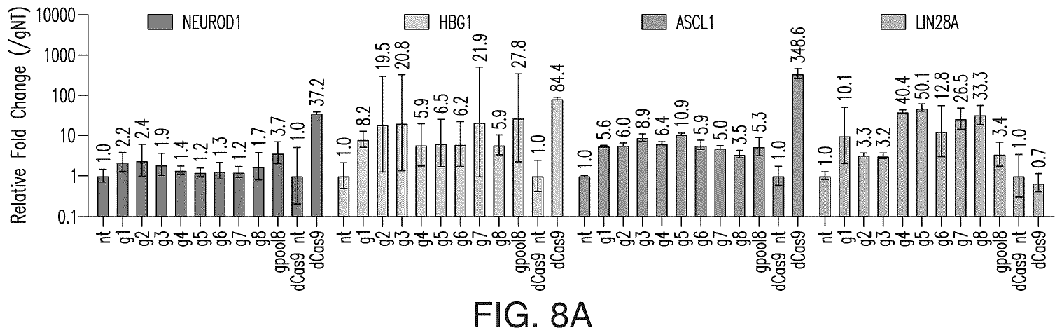

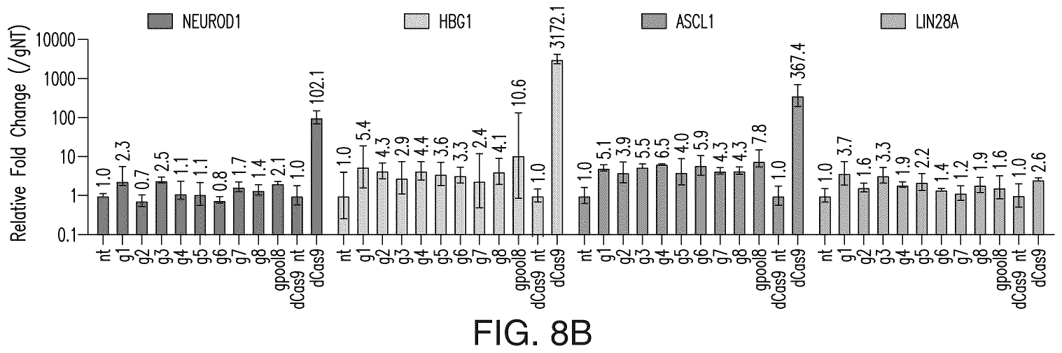

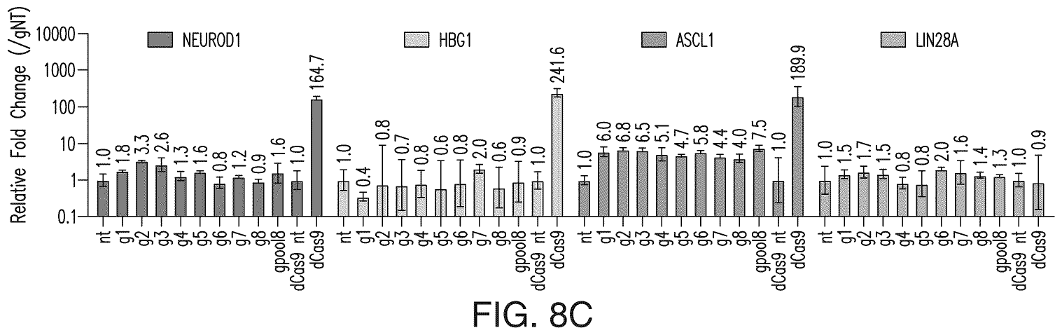

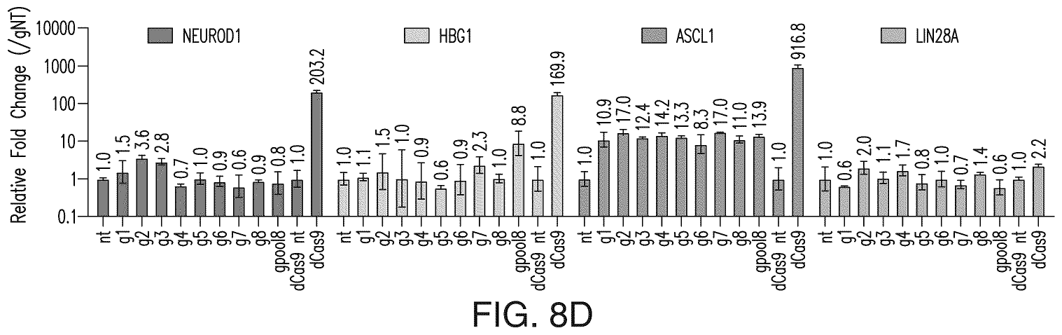

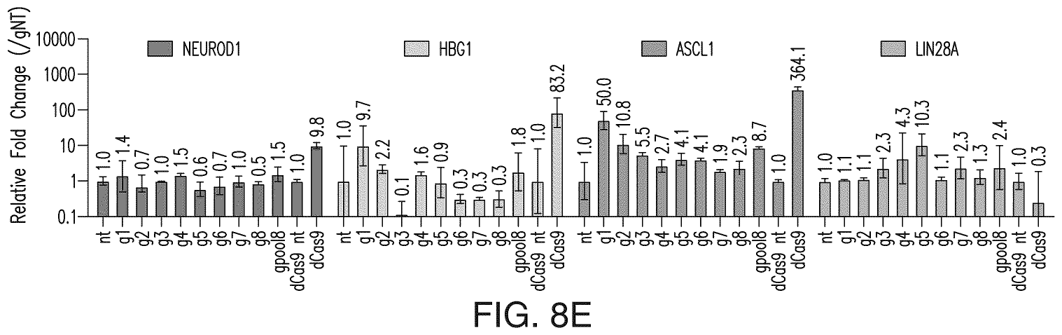

Multiple gene targets, including NEUROD1, HBG1, ASCL1, and LIN28A, were selected for testing the ability of VPR-CasM fusions to increase endogenous gene expression. A nucleic acid vector encoding VPR was fused to catalytically inactive CasM proteins at their N′ terminus with an XTEN10 linker (GSPAGSPTST SEQ ID NO: 513) and at least one CasM gRNA targeting an endogenous gene were introduced to cells via lipofection. Relative amounts of RNA, indicative of relative gene expression, were quantified with RT-qPCR. An increase of gene expression was observed with individual different gRNAs. A scramble sequence spacer, and a pooled sample were used as negative controls. A catalytically inactive “dead” Cas9 fusion, dCas9, was included as a positive control. The fusion proteins were tested for their ability to increase expression in NEUROD1, HBG1, ASCL1, and LIN28A by different VPR-CasM fusions. FIG. 8A shows the change in gene expression by CasM.286251 (D267A) (SEQ ID NO: 222) with an N terminal VPR fused by an XTEN10 linker, which demonstrated upregulation for ASCL1, HBG1 and LIN28A relative to the scrambled sequence control. FIG. 8B shows the change in gene expression by CasM. 19952 (D267A) (SEQ ID NO: 223) with an N terminal VPR fused by an XTEN10 linker, which demonstrated upregulation in ASCL1 and HBG1 and guide 3 for NEUROD1 relative to the scrambled sequence control. FIG. 8C shows the change in gene expression by CasM. 19952 (D267N) (SEQ ID NO: 224) with an N terminal VPR fused by an XTEN10 linker, which demonstrated upregulation with guides 1-8 for ASCL1 and guides 2-3 for NEUROD1 relative to the scrambled sequence control. FIG. 8D shows the change in gene expression by CasM. 19952 (E363Q) (SEQ ID NO: 225) with an N terminal VPR fused by an XTEN10 linker, which demonstrated upregulation with guides 1-8 for ASCL1 and guides 2-3 for NEUROD1 relative to the scrambled sequence control. FIG. 8E shows the change in gene expression by CasM.124070 (D326A) (SEQ ID NO: 226) with an N terminal VPR fused by an XTEN10 linker, which demonstrated upregulation for ASCL1 guide 1, HBG1 guide 1, and LIN28A guide 5 relative to the scrambled sequence control. The PAM sequence for the CasM 19952 enzymes was NTCG comprising the repeat sequence of: UGGGGCAGUUGGUUGCCCUUAGCCUGAGGCAUUUAUUGCACUCGGGAAGUAC CAUUUCUCAGAAAUGGUACAUCCAAC (SEQ ID NO: 300). The PAM sequence for the CasM 286251 enzymes was RTTR comprising the repeat sequence of: AUGGGGCAGUUGGUUGCCCUUAGCCUGAGGAAUUUAAUUCACUCGGGAAGUA CCUUUCUCAUGAAAUGGUACAUCCAAC (SEQ ID NO: 301). The PAM sequence for the CasM 124070 enzymes was TTTR comprising the repeat sequence of: ACCGCUUCACCAAGUGCUGUCCCUUAGGGGAUUAGCACUUGAGUGAAGGUGG GCUGCUUGCAUCAGCCUAAUGUCGAGAAGUGCUCUUUCGGAAAGUAACCCUC GAAACAAAUUCAUCUGAAAGAAUGAAGGAAUGCAAC (SEQ ID NO: 302). TABLE 8 denotes the spacer sequence for the designated guide TDs in the FIGS. 8A-E, the gene target, and the type of nucleases tested. The results show the catalytically inactive CasM proteins fused to VPR can increase the expression of genes.

TABLE 8
Guide sequences for Activation of Gene Expression

| ID in | | | |
|---|---|---|---|
| FIGs | | Gene | |
| 8E8A- | Spacer sequence | target | Nucleases |
| g1 | CCCCCCACUCCCCGCUGCUG (SEQ ID NO: 677) | ASCL1 | CasM.19952 |
| g2 | AAGUGGCAUCCUCUCUGAGC (SEQ ID NO: 678) | ASCL1 | CasM.19952 |
| g3 | CUUCCUCGUCUGCAGCCACA (SEQ ID NO: 679) | ASCL1 | CasM.19952 |
| g4 | ACUUUUCCUGUUUUCUCUCU (SEQ ID NO: 680) | ASCL1 | CasM.19952 |
| g5 | GGUUCCUCGGUGACCCUAGA (SEQ ID NO: 681) | ASCL1 | CasM.19952 |
| g6 | GUGACCCUAGAAAUUGGAGC (SEQ ID NO: 682) | ASCL1 | CasM.19952 |
| g7 | UCUGCAGCCACAGAAUAUGG (SEQ ID NO: 683) | ASCL1 | CasM.19952 |
| g8 | AGGAGCCACAGAGCAUUGAG (SEQ ID NO: 684) | ASCL1 | CasM.19952 |
| g1 | GAGGAGGGCGGGAGACGAGC (SEQ ID NO: | NEUROD1 | CasM.19952 |
| | 685) | | |
| g2 | UCUCCCGCCCUCCUCCGACA (SEQ ID NO: 686) | NEUROD1 | CasM.19952 |
| g3 | CCAGUUAGAGACUCCGCGGA(SEQ ID NO: 687) | NEUROD1 | CasM.19952 |
| g4 | CUCUGAUCUAGACCUAGUUA (SEQ ID NO: 688) | NEUROD1 | CasM.19952 |
| g5 | CGCCGGAAGUAGGACAGAGG (SEQ ID NO: 689) | NEUROD1 | CasM.19952 |
| g6 | AAAGGAGCGAGGACUCUUCA (SEQ ID NO: 690) | NEUROD1 | CasM.19952 |
| g7 | CUCCUUUCGAUUUCUUGUCC (SEQ ID NO: 691) | NEUROD1 | CasM.19952 |
| g8 | AUUUCUUGUCCUGACACUGG (SEQ ID NO: 692) | NEUROD1 | CasM.19952 |
| g1 | GAACAAGGCAAAGGCUAUAA (SEQ ID NO: | HBG1 | CasM.19952 |
| | 693) | | |
| g2 | AGUUAUAAUAGUGUGUGGAC (SEQ ID NO: | HBG1 | CasM.19952 |
| | 694) | | |
| g3 | AAUAUUAGUGUACUUUAGAC (SEQ ID NO: | HBG1 | CasM.19952 |
| | 695) | | |
| g4 | UUGAGCCCCUUCCUCGCUGC (SEQ ID NO: 696) | HBG1 | CasM.19952 |
| g5 | AAGGUACAUGUGCAGGAUGU (SEQ ID NO: | HBG1 | CasM.19952 |
| | 697) | | |
| g6 | GCAACCAGUAGCCCUUGCGU (SEQ ID NO: 698) | HBG1 | CasM.19952 |
| g7 | CACUUUCUUUCUUUGUCCUU (SEQ ID NO: 699) | HBG1 | CasM.19952 |
| g8 | GUGUUCAGUGGAUUAGAAAC (SEQ ID NO: | HBG1 | CasM.19952 |
| | 700) | | |
| g1 | GAGAAGAAGCUGCUACAUCU (SEQ ID NO: 701) | LIN28A | CasM.19952 |
| g2 | UUAACAAAUAUUAUUAGCAG (SEQ ID NO: | LIN28A | CasM.19952 |
| | 702) | | |
| g3 | UCCUACCCCCACCCCAUCCC (SEQ ID NO: 703) | LIN28A | CasM.19952 |
| g4 | GAGAUGGACAAUGGCCCGGG (SEQ ID NO: 704) | LIN28A | CasM.19952 |
| g5 | CUCCGUGUACCUCUGUUCCU (SEQ ID NO: 705) | LIN28A | CasM.19952 |
| g6 | GUGGAGAAGAUUGAAUUCAG (SEQ ID NO: | LIN28A | CasM.19952 |
| | 706) | | |
| g7 | UACGGGGUGCUCUCCAAGAA (SEQ ID NO: 707) | LIN28A | CasM.19952 |
| g8 | UGGGGUAAAAAGGACAAGAG (SEQ ID NO: | LIN28A | CasM.19952 |
| | 708) | | |
| g1 | AAAAGGCGGACGCACUCCGG (SEQ ID NO: 709) | ASCL1 | CasM.286251 |
| g2 | GGGGAGGGACUCCGUCCAGA (SEQ ID NO: 710) | ASCL1 | CasM.286251 |
| g3 | GAGACCAUAUUCUGUGGCUG (SEQ ID NO: 711) | ASCL1 | CasM.286251 |
| g4 | AGGUGUAUAGGUGGAAAGAC (SEQ ID NO: | ASCL1 | CasM.286251 |
| | 712) | | |
| g5 | UUCUCUUCGGGUUCCUCGGU (SEQ ID NO: 713) | ASCL1 | CasM.286251 |
| g6 | GAGCAAAUUACGAUUGAAGU (SEQ ID NO: | ASCL1 | CasM.286251 |
| | 714) | | |
| g7 | CGAUUGAAGUUUAGAAACAU (SEQ ID NO: | ASCL1 | CasM.286251 |
| | 715) | | |
| g8 | AAGUUUAGAAACAUGGUUGG (SEQ ID NO: | ASCL1 | CasM.286251 |
| | 716) | | |
| g1 | UCGGAGGAGGGCGGGAGACG (SEQ ID NO: | NEUROD1 | CasM.286251 |
| | 717) | | |
| g2 | AUCUCUCCUGCGGGUAAAAA (SEQ ID NO: 718) | NEUROD1 | CasM.286251 |
| g3 | GCUUUUCCCUUCCUUCCCUC (SEQ ID NO: 719) | NEUROD1 | CasM.286251 |
| g4 | ACAUUAGCUUUUCCCUUCCU (SEQ ID NO: 720) | NEUROD1 | CasM.286251 |
| g5 | ACUAGGUCUAGAUCAGAGCG (SEQ ID NO: 721) | NEUROD1 | CasM.286251 |
| g6 | GCGCCAAAGGAUGGCUUCUC (SEQ ID NO: 722) | NEUROD1 | CasM.286251 |
| g7 | GGAGAAGCCAUCCUUUGGCG (SEQ ID NO: 723) | NEUROD1 | CasM.286251 |
| g8 | GGGAACUAAUCUCAACGCUG (SEQ ID NO: 724) | NEUROD1 | CasM.286251 |
| g1 | GUCAAGUUUGCCUUGUCAAG (SEQ ID NO: 725) | HBG1 | CasM.286251 |
| g2 | GCCAGCCUUGCCUUGACCAA (SEQ ID NO: 726) | HBG1 | CasM.286251 |
| g3 | GUCAAGGCAAGGCUGGCCAA (SEQ ID NO: 727) | HBG1 | CasM.286251 |
| g4 | AGAUAGUGUGGGGAAGGGGC (SEQ ID NO: | HBG1 | CasM.286251 |
| | 728) | | |
| g5 | GCAGUGGUUUCUAAGGAAAA (SEQ ID NO: | HBG1 | CasM.286251 |
| | 729) | | |
| g6 | GAGAAAAACUGGAAUGACUG (SEQ ID NO: | HBG1 | CasM.286251 |
| | 730) | | |
| g7 | GUACAUGCUUUAGCUUUAAA (SEQ ID NO: | HBG1 | CasM.286251 |
| | 731) | | |
| g8 | AGAGAUAAUGGCAAAAGUCA (SEQ ID NO: | HBG1 | CasM.286251 |
| | 732) | | |
| g1 | GUUCGGAGAAGAAGCUGCUA (SEQ ID NO: | LIN28A | CasM.286251 |
| | 733) | | |
| g2 | UGCGGGGGAAGAUGUAGCAG (SEQ ID NO: | LIN28A | CasM.286251 |
| | 734) | | |
| g3 | UCUUUUAGAAUUUGGGAGCC (SEQ ID NO: | LIN28A | CasM.286251 |
| | 735) | | |
| g4 | GGUCAUUGUCUUUUAGAAUU (SEQ ID NO: | LIN28A | CasM.286251 |
| | 736) | | |
| g5 | UGGGGGAGGGCCGGAGCUGG (SEQ ID NO: | LIN28A | CasM.286251 |
| | 737) | | |
| g6 | UGCGUGUGGGGAGGGGGUGU (SEQ ID NO: | LIN28A | CasM.286251 |
| | 738) | | |
| g7 | GGGGAGGGAGGUGUGAGCCU (SEQ ID NO: | LIN28A | CasM.286251 |
| | 739) | | |
| g8 | GCCAGCGCCGCCAGGCUCAC (SEQ ID NO: 740) | LIN28A | CasM.286251 |
| g1 | GGGAGUGGGUGGGAGGAAGA (SEQ ID NO: | ASCL1 | CasM.124070 |
| | 741) | | |
| g2 | CAAGGAGCGGGAGAAAGGAA (SEQ ID NO: | ASCL1 | CasM.124070 |
| | 742) | | |
| g3 | UUGUUGCAGUGCGUGCGCCU (SEQ ID NO: 743) | ASCL1 | CasM.124070 |
| g4 | UUCAGCCGGGAGUCCGGCAC (SEQ ID NO: 744) | ASCL1 | CasM.124070 |
| g5 | GGAAGGGGGUGGGGGGCGUC (SEQ ID NO: | ASCL1 | CasM.124070 |
| | 745) | | |
| g6 | UCCCUCCUGUGACGCCCCCC (SEQ ID NO: 746) | ASCL1 | CasM.124070 |
| g7 | CUUCAAGUUCUUAGUAGAAU (SEQ ID NO: | ASCL1 | CasM.124070 |
| | 747) | | |
| g8 | GGCAGAAAAAAUGGCACAGG (SEQ ID NO: | ASCL1 | CasM.124070 |
| | 748) | | |
| g1 | UAUGCCGCGGAGCGCUCCAU (SEQ ID NO: 749) | NEUROD1 | CasM.124070 |
| g2 | UGGCCAGAAGAAAGUGGCCC (SEQ ID NO: 750) | NEUROD1 | CasM.124070 |
| g3 | CCCGCAGGAGAGAUUAACCC (SEQ ID NO: 751) | NEUROD1 | CasM.124070 |
| g4 | AGGGAAGGAAGGGAAAAGCU (SEQ ID NO: | NEUROD1 | CasM.124070 |
| | 752) | | |
| g5 | GCGCCAAUGAUCAAAGCGUC (SEQ ID NO: 753) | NEUROD1 | CasM.124070 |
| g6 | AUCAUUGGCGCCAAAGGAUG (SEQ ID NO: 754) | NEUROD1 | CasM.124070 |
| g7 | CUUAGAGGGGCCGACGGAGA (SEQ ID NO: 755) | NEUROD1 | CasM.124070 |
| g8 | UAUGGCGUAUAUGUUUGCUU (SEQ ID NO: | NEUROD1 | CasM.124070 |
| | 756) | | |
| g1 | UUCUUCAUCCCUAGCCAGCC (SEQ ID NO: 757) | HBG1 | CasM.124070 |
| g2 | CCUUGUCAAGGCUAUUGGUC (SEQ ID NO: 758) | HBG1 | CasM.124070 |
| g3 | GCCAGGGACCGUUUCAGACA (SEQ ID NO: 759) | HBG1 | CasM.124070 |
| g4 | CAUUGAGAUAGUGUGGGGAA (SEQ ID NO: | HBG1 | CasM.124070 |
| | 760) | | |
| g5 | UAGCCUUUGCCUUGUUCCGA (SEQ ID NO: 761) | HBG1 | CasM.124070 |
| g6 | CCUUGUUCCGAUUCAGUCAU (SEQ ID NO: 762) | HBG1 | CasM.124070 |
| g7 | UUCUUCCCUUUAGCUAGUUU (SEQ ID NO: 763) | HBG1 | CasM.124070 |
| g8 | GCUAGUUUCCUUCUCCCAUC (SEQ ID NO: 764) | HBG1 | CasM.124070 |
| g1 | AAAAGCCGUGGGCCCUCCCA (SEQ ID NO: 765) | LIN28A | CasM.124070 |
| g2 | GGAGCCUUUGAAAAGCCGUG (SEQ ID NO: 766) | LIN28A | CasM.124070 |
| g3 | GAAUUUGGGAGCCUUUGAAA (SEQ ID NO: | LIN28A | CasM.124070 |
| | 767) | | |
| g4 | ACACCCCCUCCCCACACGCA (SEQ ID NO: 768) | LIN28A | CasM.124070 |
| g5 | AGAUCCUGCACUUUGGACUC (SEQ ID NO: 769) | LIN28A | CasM.124070 |
| g6 | GGGACCCCUACUGAGUCCAA (SEQ ID NO: 770) | LIN28A | CasM.124070 |
| g7 | GACUCAGUAGGGGUCCCCAA (SEQ ID NO: 771) | LIN28A | CasM.124070 |
| g8 | AAACUACCCCCCCCACCAGG (SEQ ID NO: 772) | LIN28A | CasM.124070 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-96. (canceled)

97. A composition comprising an engineered polypeptide or a nucleic acid encoding the engineered polypeptide, wherein the engineered polypeptide comprises an amino acid sequence that is at least 95% identical to SEQ ID NO: 6, and wherein the amino acid sequence comprises an amino acid substitution at E567 relative to SEQ ID NO: 6.

98. The composition of claim 97, wherein the engineered polypeptide has reduced nuclease activity relative to the nuclease activity of a polypeptide that is 100% identical to SEQ ID NO: 6.

99. The composition of claim 97, wherein the amino acid substitution is selected from E567A and E567Q.

100. The composition of claim 97, wherein the amino acid substitution is E567Q.

101. The composition of claim 97, wherein the engineered polypeptide comprises the amino acid sequence of SEQ ID NO: 219.

102. The composition of claim 97, wherein the amino acid sequence is at least 98% identical to SEQ ID NO: 6.

103. The composition of claim 97, further comprising a guide nucleic acid.

104. The composition of claim 103, wherein the guide nucleic acid comprises a nucleotide sequence that is at least 90% identical to SEQ ID NO: 784.

105. The composition of claim 97, wherein the nucleic acid encoding the engineered polypeptide is an RNA.

106. The composition of claim 105, further comprising a lipid nanoparticle (LNP), wherein the LNP contains the RNA.

107. The composition of claim 106, further comprising a guide nucleic acid, wherein the LNP contains the guide nucleic acid.

108. The composition of claim 97, wherein the nucleic acid encoding the engineered polypeptide is an adeno-associated viral vector (AAV).

109. The composition of claim 97, further comprising a fusion partner protein that is linked to the engineered polypeptide, wherein the fusion partner protein is capable of modifying or regulating the expression of a nucleic acid.

110. The composition of claim 97, wherein the nucleic acid encoding the engineered polypeptide is linked to a nucleic acid encoding a fusion partner protein.

111. The composition of claim 110, wherein the fusion partner protein comprises a reverse transcriptase.

112. The composition of claim 110, wherein the fusion partner protein comprises a deaminase.

113. The composition of claim 110, wherein the fusion partner protein comprises a transcriptional inhibitor.

114. The composition of claim 110, wherein the fusion partner protein comprises a transcriptional activator.

115. The composition of claim 110, wherein the fusion partner protein comprises a DNA methyltransferase.

116. The composition of claim 110, wherein the fusion partner protein is selected from the group consisting of: DNMT3A, DNMT3L, EZH2, KRAB/KOX1, and ZIM3, and any functional fragment thereof, and any combination thereof.

117. The composition of claim 110, wherein the fusion partner protein comprises an amino acid sequence that is at least 90% identical to an amino acid sequence selected from SEQ ID NOs: 410, 411 and 413-415.

118. A composition comprising a nucleic acid, wherein the nucleic acid encodes:
a) an engineered polypeptide, wherein the engineered polypeptide comprises an amino acid sequence that is at least 95% identical to SEQ ID NO: 6, and wherein the amino acid sequence comprises an amino acid substitution of E567Q relative to SEQ ID NO: 6; and
b) a DNA methyltransferase.

119. The composition of claim 118, wherein the DNA methyltransferase is selected from DNMT3A and DNMT3L.

120. The composition of claim 119, wherein the nucleic acid comprises an RNA.

121. The composition of claim 120, further comprising a lipid nanoparticle (LNP), wherein the LNP contains the RNA.

122. The composition of claim 121, further comprising a guide nucleic acid, wherein the LNP contains the guide nucleic acid.

123. The composition of claim 118, wherein the nucleic acid comprises an adeno-associated viral (AAV) vector.

124. The composition of claim 122, wherein the guide nucleic acid comprises a nucleotide sequence that is at least 95% identical to SEQ ID NO: 784.

125. A method of modifying a target nucleic acid comprising contacting the target nucleic acid with the composition of claim 109.

126. A method of regulating expression of a target nucleic acid comprising contacting the target nucleic acid with the composition of claim 109.
